(19) United States
(12) Patent Application Publication
(10) Pub. No.: US 2026/0053744 A1
(43) Pub. Date: Feb. 26, 2026

(54) NANODELIVERY SYSTEMS FOR EXTENDED RELEASE OF ANTIBODIES

(71) Applicant: University of Maryland, Baltimore, Baltimore, MD (US)

(72) Inventors: Mohammad Sajadi, Cockeysville, MD (US); Abdolrahim Abbasi, Bethesda, MD (US); Maryam Karimi, Baltimore, MD (US)

(21) Appl. No.: 19/249,961

(22) Filed: Jun. 25, 2025

Related U.S. Application Data
(60) Provisional application No. 63/663,881, filed on Jun. 25, 2024.

(51) Int. Cl. A61K 9/1271; A61K 9/00; A61K 39/42

(52) U.S. Cl. CPC A61K 9/1271; A61K 9/0019; A61K 39/42

(57) ABSTRACT

The present invention provides a liposomal pharmaceutical formulation for subcutaneous or intravenous administration of antibodies, comprising one or more phospholipids; one or more antibodies; and a pharmaceutically acceptable vehicle, wherein the one or more phospholipids form liposomes that encapsulate the one or more antibodies, wherein the liposomes have a polydispersity index (PDI) below 0.2 and at least 90% of the liposomes have a size less than 125 nm.

### CROSS-REFERENCE TO RELATED APPLICATIONS

This application claims the benefit of U.S. Provisional Appl. No. 63/663,881, filed on Jun. 25, 2024, the contents of which are hereby incorporated by reference in their entirety.

### INCORPORATION-BY-REFERENCE OF MATERIAL SUBMITTED ELECTRONICALLY

Incorporated by reference in its entirety herein is a computer-readable sequence listing submitted concurrently herewith and identified as follows: One 909,836 Byte XML file named “sequence_listing.xml,” created on Jun. 23, 2025.

### FIELD OF THE INVENTION

The invention generally relates to the field of medicine and to compositions and methods for providing sustained release and extending half-life of administered antibodies, specifically monoclonal antibodies that can be used for HIV prevention and treatment.

### BACKGROUND OF THE INVENTION

Monoclonal antibodies (mAbs) convey a significant breakthrough in medicine. The origins of antibody-based disease treatment can be traced back to the pioneering work of Emil Adolf von Behring in the 1890s (Grundbacher F J, Immunol Today, (1992), 13:188-90). His groundbreaking experiments revealed the transferable immunity between animals through serum containing antibodies against diphtheria and tetanus toxins (Grundbacher F J, Immunol Today, (1992), 13:188-90). However, it was in the 1960s that we achieved a more profound knowledge of the structural characteristics of antibodies, thanks to the work of scientists like Gerald Edelman and Rodney Porter (Ribatti D., Immunology Letters, (2015), 164:72-5). This knowledge paved the way for the monumental discovery of monoclonal antibodies (mAbs) production methods in the following decade, notably the hybridoma technology pioneered by Köhler and Milstein in 1975 (Leavy O., Nature Immunology, (2016), 17:S13-S).

The FDA's approval of Orthoclone in 1986 marked a significant milestone in monoclonal antibody therapeutics to prevent organ rejection post-transplantation (Ecker et al., mAbs, (2015), 7:9-14). Since then, the FDA has continued to play a pivotal role in advancing the field of mAbs by approving over 100 novel antibodies for a diverse range of conditions. These approvals span various therapeutic areas, including cancer, autoimmune diseases, infectious diseases, and inflammatory conditions (Quinteros et al., Nanostructures for Drug Delivery, (2017): 807-33. doi: 10.1016/B978-O-323-46143-6.00025-7. Epub 2017 Mar. 31).

MAbs are typically given through injection rather than orally due to their poor absorption and degradation in the digestive system. Injection methods include intramuscular, intravenous (IV), or subcutaneous (Sc) routes (Awwad S, Pharmaceutics, (2018), 10(3)). SC delivery is widely accepted as safe, effective, and preferable by patients and healthcare providers. Compared to IV administration, SC offers several advantages, such as fixed dosing, decreased healthcare resource usage, decreased healthcare provider time, and reduced clinical costs (Davis et al., Clinical Pharmacology & Therapeutics, (2024), 115:422-39). Patients often favor SC delivery due to its shorter administration time and the ability to self-administer or receive support from caregivers outside of infusion centers, lessening the treatment burden and enhancing quality of life (Matucci et al., Respir Res, (2018), 19:154). Furthermore, SC injection enables treatment for patients with difficult venous access and helps preserve their veins. However, a significant downside of subcutaneous injection compared to intravenous delivery is its lower bioavailability and potential for immunogenicity (Matucci et al., Respir Res, (2018), 19:154, Sánchez-Félix et al., Advanced Drug Delivery Reviews, (2020), 167:66-77).

One of the most highly investigated classes of nanomaterial-based drug delivery carriers are liposomes. Liposomes are self-assembled vesicles constructed from lipids, which organize into single or multiple bilayers encapsulating a central aqueous compartment (Liposome Drug Products: Chemistry, Manufacturing, and Controls; Human Pharmacokinetics and Bioavailability; and Labeling Documentation, (2018)). They serve as effective drug carriers, possessing remarkable attributes such as safeguarding encapsulated compounds from degradation within the body, prolonging the drug's effective duration, regulating the controlled release of drug molecules, and demonstrating favorable biocompatibility and safety features (Niu M et al., European journal of pharmaceutics and biopharmaceutics, (2012), 81:265-72, Wang N et al., Colloids and Surfaces B: Biointerfaces, (2009), 69:232-8). Moreover, liposomes can accurately transport their cargo to the affected area via passive and active targeting mechanisms, thereby reducing overall systemic side effects, increasing the maximum acceptable dosage, and enhancing the therapeutic advantages (Kong et al., Journal of Advanced Research, (2023), 44:119-33, Liu P et al., Molecules, (2022), 27:1372). This background information is provided for informational purposes only. No admission is necessarily intended, nor should it be construed, that any of the preceding information constitutes prior art against the present invention.

### SUMMARY OF THE INVENTION

It is to be understood that both the foregoing general description of the embodiments and the following detailed description are exemplary, and thus do not restrict the scope of the embodiments.

In one aspect, the invention provides a liposomal pharmaceutical formulation for subcutaneous or intravenous administration of antibodies, comprising one or more phospholipids; one or more antibodies; and a pharmaceutically acceptable vehicle, wherein the one or more phospholipids form liposomes that encapsulate the one or more antibodies, wherein the liposomes have a polydispersity index (PDI) below 0.2 and at least 90% of the liposomes have a size less than 125 nm.

Advantageously, in some embodiments, the liposomal pharmaceutical formulation is physically stable, with minimal or no aggregation, no visible sedimentation, and minimal loss of encapsulated antibody. Additionally, in some embodiments, the liposomal pharmaceutical formulations preserve their functional properties, including sustained release, bioactivity, and favorable pharmacokinetics.

In some embodiments, the liposomal pharmaceutical formulation of the antibodies injected subcutaneously or intravenously increase the AUC, bioavailability, and half-life of the antibody compared to free antibody injected subcutaneously or intravenously.

In some embodiments, the liposomal pharmaceutical formulation is less immunogenic than formulations of free antibodies upon subcutaneous or intravenous administration in a subject, and results in a reduction of anti-drug antibody formation in the subject.

In some embodiments, the liposomes are embedded or encapsulated in an injectable matrix. In some embodiments, the liposomes are embedded in microspheres. In some embodiments, the liposomes are embedded or encapsulated in an injectable silica matrix. In some embodiments, the liposomes are embedded in an injectable hydrogel matrix.

In some embodiments, the injectable microspheres comprise poly(lactic-co-glycolic acid) (PGLA). In some embodiments, the microspheres are embedded in a hydrogel.

In some embodiments, the injectable hydrogel matrix comprises poly(vinyl alcohol) (PVA). In some embodiments, the injectable hydrogel matrix comprises a blend of PVA and gelatin.

In some embodiments, the injectable matrix is chemically or physically crosslinked. In some embodiments, the injectable matrix is crosslinked using genipin.

In another aspect, the invention provides a method of treating or preventing a disease or condition in a subject, comprising administering subcutaneously to the subject an effective amount of the liposomal pharmaceutical formulation as provided herein.

In some embodiments, the disease or condition is HIV infection.

In some embodiments, release of the antibody into the subject's plasma is delayed and then sustained over a period of time.

In some embodiments, the antibody achieves a peak plasma concentration immediately, e.g., in the case of intravenous administration, or 7 days following administration in the case of subcutaneous injection in the subject. In some embodiments, the antibody achieves a peak plasma concentration between 7 and 14 days following administration in the subject. In some embodiments, the antibody has a half-life of at least 12 days to one or several months, e.g., three months, in the subject. In some embodiments, the antibody has a half-life in the subject that is at least 30% greater than the half-life of the free antibody in a control subject administered an intravenous injection of the free antibody. In some embodiments, the antibody has a half-life in the subject that is from about 30% to about 400% greater than the half-life of the free antibody in a control subject administered an intravenous injection of the free antibody.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE FIGURES

The skilled artisan will understand that the drawings, described below, are for illustration purposes only. The drawings are not intended to limit the scope of the present teachings in any way.

FIG. 1. Transmission electron microscopy (TEM) images of negatively stained liposomes. (A) The PEGylated liposomes containing N49P9.6 FR-LS and (B) Non-PEGylated liposomes containing mAb of negatively stained liposomes containing N49P9.6 FR-LS.

FIG. 2. 24 hour in vitro release of mAb-loaded liposomes. PEGylated and non-PEGylated liposomes loaded with N49P9.6 FR-LS were placed in dialysis cassettes and inserted in 50% sterile human plasma and 5% sterilized dextrose (pH 7.4) at 37° C. over 24h. The control was free mAb in the solution. Liposome encapsulation led to less release of mAbs after 24 hours (P value <0.0001 free mAb vs. PEGylated liposome, and free mAb vs. non-PEGylated liposome).

FIG. 3. 42 day in vitro release of mAb-loaded liposomes. PEGylated and non-PEGylated liposomes loaded with N49P9.6 FR-LS were placed in 50% sterile human plasma and 5% sterilized dextrose (pH 7.4) at 37° C., with gentle stirring in sterile conditions. The percentage of retained encapsulated antibodies was measured at various time points by analyzing two samples at each interval. The presented data are expressed as mean±SD, providing insights into the release dynamics and stability of the liposomal formulations over the specified incubation period.

FIG. 4. Cytotoxicity of liposomal formulations. A) The viability of HEMC-1 WT, HEMC-1 FcRn cell lines in the presence of liposomal formulations or free antibody was measured with the MTT assay. Cells were treated with 0.25 ug, 0.5 ug, or lug of free mAb or equivalent amount encapsulated in liposomes and cell viability measured at 24 and 48 hours. B) The viability of HEMC-1 WT, HEMC-1 FcRn cell lines in the presence of liposomal formulations or free antibody was measured with the Caspase-3 apoptosis assay. Wildtype and FcRn HMEC1 cells were trypsinized after treatment with mAb-loaded liposomes, free mAb, or heated to 55° C. for 7 min (apoptosis/dead induction control), or no treatment (negative control), and assessed for binding to caspase-3 inhibitor DEVD-FMK conjugated to sulfo-rhodamine (Red-DEVD-FMK). In this assay, free antibody and liposomal formulations did not affect cell viability, and only heat treated cells led to apoptosis.

FIG. 5. In vitro TLR Reporter Assay. RAW-BLUE™ mouse macrophage reporter cells were exposed to antibodies and liposome-encapsulated antibody nanoparticles, along with positive controls including Resiqumed and LPS. Results demonstrate that none of the antibodies or liposomal particles elicited macrophage activation. Conversely, positive controls effectively activated the macrophages.

FIG. 6. In vitro cellular uptake of antibodies and liposomes in HMEC-1 FcRn cells. HMEC-1 FcRn cells were treated with antibodies and liposomes for durations of 1, 3, 6, 9, and 24 hours. Subsequently, the cells were harvested, fixed with PFA for 10 minutes at room temperature, and subjected to intracellular staining to assess the antibody content. Flow cytometry analysis was then performed. The results demonstrate the uptake of both liposomes and antibodies by the cells. Furthermore, it is observed that PEGylated liposomes containing antibodies exhibit prolonged retention within the cells compared to antibodies alone and non-PEGylated liposomes.

FIG. 7. In vivo pharmacokinetics of IV and SC liposomal formulations. Groups of 5 hFcRn mice were injected SC or IV with free mAb or mAb loaded liposomes and mAb levels (ug/ml) measured over time with Elisa. A) PK of free mAbs vs. non-PEG liposomes loaded with N49P9.6-FR-LS shown. B) PK of free mAbs vs. PEG liposomes loaded with N49P9.6-FR-LS shown. C) PK of free mAbs vs. PEG liposomes loaded with PGT121 shown. Free mAb controls in panels A and B are identical.

FIG. 8. Binding and function of mAbs after liposomal formulation and injection in mice. A) Binding Assay of Plasma Samples to Raji-Env Cells. The binding assay depicted illustrates the interaction between plasma samples obtained from groups of three mice two weeks post-injection and Raji-Env cells. The data indicate that plasma samples from mice injected with liposomes exhibit robust binding to Raji-Env cells, comparable to the binding observed with plasma derived from mice injected with antibodies. B) Neutralization of HIV-1 by mouse plasma. Plasma from mice injected with free mAb or mAb-loaded liposomes tested for neutralization against HIV-1 BaL.26. Plasma from mice injected with free mAb or mAb-loaded liposomes shows potent anti-HIV activity, whereas none seen in mouse injected with the free mAb Synagis (an antibody against a different virus, RSV).

FIG. 9. Rescue of SC administered bNAbs with liposomes. hFcRn transgenic mice injected SC or IV with PGT121 alone or formulated in pegylated liposomes, and bled weekly with concentration of antibody measured.

FIG. 10. Schematic showing the strategies employed to increase mAb or bNAb half-life. Graph on bottom right shows theoretical PK of an eN49 bNAb injected subcutaneously with Fc engineering, packaging in liposomes and slow delivery with hydrogel. The dotted red line shows serum level needed to have >90% protective efficacy (single bNAb) based on geometric mean IC80 values of ebNAb N49P9.6-FR. ebNAb=engineered broadly neutralizing antibody; NDDS=nano drug delivery system; SC=subcutaneous injection; PK=pharmacokinetics; PT80=the 80% inhibitory dilution titer. t_{1/2}=half-life

FIG. 11. 21 day in vitro release of mAb-loaded liposomes in different hydrogel matrices. PEG liposomes loaded with N49P9.6-FR-LS were embedded in 3 different hydrogel matrices (non-crosslinked PVA; genipin-crosslinked PVA; or gelatin hydrogel with PLGA microspheres) and then placed in 50% sterile human plasma and 5% sterilized dextrose (pH 7.4) at 37° C., with gentle stirring in sterile conditions. At each time point, percentage of retained encapsulated antibodies was determined by testing 2 independent samples. The presented data are expressed as mean±SD, providing insights into the release dynamics and stability of the liposomal formulations over the specified incubation period.

FIG. 12. Cytotoxicity of mAb-loaded liposomal hydrogel formulations. A) The viability of HEMC-1 WT, HEMC-1 FcRn cell lines in the presence of liposomal hydrogel formulations (non-crosslinked PVA; genipin-crosslinked PVA; or gelatin hydrogel with PLGA microspheres) or free antibody was measured with the MTT assay. Cells were treated with 0.25 ug, 0.5 ug, or lug of free mAb or equivalent amount encapsulated liposome/hydrogel and cell viability measured at 24 and 48 hours. B) In vitro TLR Reporter Assay. RAW-BLUE™ mouse macrophage reporter cells were treated with antibodies and the hydrogel formulation, alongside positive controls such as Resiquimod and LPS. The results showed that neither the antibodies nor the hydrogel induced macrophage activation, whereas the positive controls triggered a robust activation response. ***=P<0.001

FIG. 13. In vivo pharmacokinetics of mAb-loaded liposomal hydrogel formulations. Groups of 5 hFcRn mice were injected with free mAb, mAb loaded liposomes, or mAb-loaded liposomal hydrogel formulations (non-crosslinked PVA; genipin-crosslinked PVA; or gelatin hydrogel with PLGA microspheres) and mAb levels (ug/ml) measured over time with ELISA.

### DETAILED DESCRIPTION OF THE INVENTION

The present disclosure describes liposomal formulations of antibodies for subcutaneous or intravenous administration and enhancement of antibody pharmacokinetics through subcutaneous or intravenous liposomal delivery.

Reference will now be made in detail to the presently preferred embodiments of the invention which, together with the drawings and the following examples, serve to explain the principles of the invention. These embodiments describe in sufficient detail to enable those skilled in the art to practice the invention, and it is understood that other embodiments may be utilized, and that structural, biological, and chemical changes may be made without departing from the spirit and scope of the present invention. Unless defined otherwise, all technical and scientific terms used herein have the same meanings as commonly understood by one of ordinary skill in the art.

The practice of the present invention employs, unless otherwise indicated, conventional techniques of molecular biology (including recombinant techniques), microbiology, cell biology, biochemistry and immunology, which are within the skill of the art. Such techniques are explained fully in the literature. See, e.g., Sambrook et al. Molecular Cloning: A Laboratory Manual, 2^{nd} edition (1989); Current Protocols in Molecular Biology (F. M. Ausubel et al. eds. (1987)); the series Methods in Enzymology (Academic Press, Inc.); PCR: A Practical Approach (M. MacPherson et al. IRL Press at Oxford University Press (1991)); PCR 2: A Practical Approach (M. J. MacPherson, B. D. Hames and G. R. Taylor eds. (1995)); Antibodies, A Laboratory Manual (Harlow and Lane eds. (1988)); Using Antibodies, A Laboratory Manual (Harlow and Lane eds. (1999)); and Animal Cell Culture (R. I. Freshney ed. (1987)). Definitions of common terms in molecular biology may be found, for example, in Benjamin Lewin, Genes VII, published by Oxford University Press, 2000 (ISBN 019879276X); Kendrew et al. (eds.); The Encyclopedia of Molecular Biology, published by Blackwell Publishers, 1994 (ISBN 0632021829); and Robert A. Meyers (ed.), Molecular Biology and Biotechnology: a Comprehensive Desk Reference, published by Wiley, John & Sons, Inc., 1995 (ISBN 0471186341).

For the purpose of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used). The use of “or” means “and/or” unless stated otherwise. As used in the specification and claims, the singular form “a,” “an” and “the” include plural references unless the context clearly dictates otherwise. For example, the term “a cell” includes a plurality of cells, including mixtures thereof. The use of “comprise,” “comprises,” “comprising,” “include,” “includes,” and “including” are interchangeable and not intended to be limiting. Furthermore, where the description of one or more embodiments uses the term “comprising,” those skilled in the art would understand that, in some specific instances, the embodiment or embodiments can be alternatively described using the language “consisting essentially of” and/or “consisting of.”

Abbreviations for amino acids are used throughout this disclosure and follow the standard nomenclature known in the art. For example, as would be understood by those of ordinary skill in the art, Alanine is Ala or A; Arginine is Arg or R; Asparagine is Asn or N; Aspartic Acid is Asp or D; Cysteine is Cys or C; Glutamic acid is Glu or E; Glutamine is Gln or Q; Glycine is Gly or G; Histidine is His or H; Isoleucine is Ile or I; Leucine is Leu or L; Lysine is Lys or K; Methionine is Met or M; Phenylalanine is Phe or F; Proline is Pro or P; Serine is Ser or S; Threonine is Thr or T; Tryptophan is Trp or W; Tyrosine is Tyr or Y; and Valine is Val or V.

As used herein, the term “about” means plus or minus 10% of the numerical value of the number with which it is being used.

The term “antibody” means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, antibody fragments (such as Fab, Fab′, F(ab′)2, and Fv fragments, dual affinity retargeting antibodies (DART)), single chain Fv (scFv) mutants, multispecific antibodies such as bispecific and trispecific antibodies generated from at least two intact antibodies, chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity.

In some embodiments, an antibody can be of any the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as toxins, radioisotopes, etc.

The basic four-chain antibody unit is a heterotetrameric glycoprotein composed of two identical light (L) chains and two identical heavy (H) chains. An IgM antibody consists of 5 basic heterotetramer units along with an additional polypeptide called J chain, and therefore contain 10 antigen binding sites, while secreted IgA antibodies can polymerize to form polyvalent assemblages comprising 2-5 of the basic 4-chain units along with J chain. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable region (V_{H}) followed by three constant domains (C_{H}) for each of the Q and Q chains and four C_{H} domains for □ and □ isotypes. Each L chain has at the N-terminus, a variable region (V_{L}) followed by a constant domain (C_{L}) at its other end. The V_{L} is aligned with the V_{H} and the C_{L} is aligned with the first constant domain of the heavy chain (C_{H1}). Particular amino acid residues are believed to form an interface between the light chain and heavy chain variable regions. The pairing of a V_{H} and V_{L} together forms a single antigen-binding site. For the structure and properties of the different classes of antibodies, see, e.g., Basic and Clinical Immunology, 8th edition, Daniel P. Stites, Abba I. Terr and Tristram G. Parslow (eds.), Appleton & Lange, Norwalk, Conn., 1994, page 71, and Chapter 6.

The L chain from any vertebrate species can be assigned to one of two clearly distinct types, called kappa (□) and lambda (□), based on the amino acid sequences of their constant domains (C_{L}). Depending on the amino acid sequence of the constant domain of their heavy chains (C_{H}), immunoglobulins can be assigned to different classes or isotypes. There are five classes of immunoglobulins: IgA, IgD, IgE, IgG, and IgM, having heavy chains designated alpha (□), delta (□□, epsilon (□), gamma (□) and mu (□) respectively. The □ and □ classes are further divided into subclasses on the basis of relatively minor differences in C_{H} sequence and function, e.g., humans express the following subclasses: IgG1, IgG2, IgG3, IgG4, IgA1, and IgA2.

The terms “antigen” or “immunogen” are used interchangeably to refer to a substance, typically a protein, which is capable of inducing an immune response in a subject. The term also refers to proteins that are immunologically active in the sense that once administered to a subject (either directly or by administering to the subject a nucleotide sequence or vector that encodes the protein) is able to evoke an immune response of the humoral and/or cellular type directed against that protein.

The term “antigen binding fragment” or antibody fragment refers to a portion of an intact antibody and comprises the antigenic determining variable regions of an intact antibody. Examples of antigen binding fragment include, but are not limited to Fab, Fab′, F(ab′)2, and Fv fragments, linear antibodies, single chain antibodies, and multispecific antibodies formed from antibody fragments.

A “monoclonal antibody” refers to a homogeneous antibody population involved in the highly specific recognition and binding of a single antigenic determinant, or epitope. This is in contrast to polyclonal antibodies that typically include different antibodies directed against different antigenic determinants. The term “monoclonal antibody” encompasses both intact and full-length monoclonal antibodies as well as antibody fragments (such as Fab, Fab′, F(ab′)2, Fv), single chain (scFv) mutants, fusion proteins comprising an antibody portion, and any other modified immunoglobulin molecule comprising an antigen recognition site. Furthermore, “monoclonal antibody” refers to such antibodies made in any number of manners including but not limited to by hybridoma, phage selection, recombinant expression, and transgenic animals.

The term “humanized antibody” refers to forms of non-human (e.g. murine) antibodies that are specific immunoglobulin chains, chimeric immunoglobulins, or fragments thereof that contain minimal non-human (e.g., murine) sequences. Typically, humanized antibodies are human immunoglobulins in which residues from the complementary determining region (CDR) are replaced by residues from the CDR of a non-human species (e.g. mouse, rat, rabbit, hamster) that have the desired specificity, affinity, and capability (Jones et al., 1986, Nature, 321:522-525; Riechmann et al., 1988, Nature, 332:323-327; Verhoeyen et al., 1988, Science, 239:1534-1536). In some instances, the Fv framework region (FR) residues of a human immunoglobulin are replaced with the corresponding residues in an antibody from a non-human species that has the desired specificity, affinity, and capability. The humanized antibody can be further modified by the substitution of additional residues either in the Fv framework region and/or within the replaced non-human residues to refine and optimize antibody specificity, affinity, and/or capability. In general, the humanized antibody will comprise substantially all of at least one, and typically two or three, variable domains containing all or substantially all of the CDR regions that correspond to the non-human immunoglobulin whereas all or substantially all of the FR regions are those of a human immunoglobulin consensus sequence. The humanized antibody can also comprise at least a portion of an immunoglobulin constant region or domain (Fc), typically that of a human immunoglobulin. Examples of methods used to generate humanized antibodies are described in U.S. Pat. No. 5,225,539 or 5,639,641.

A “variable region” of an antibody refers to the variable region of the antibody light chain or the variable region of the antibody heavy chain, either alone or in combination. The variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen-binding site of antibodies. The term “hypervariable region” when used herein refers to the amino acid residues of an antibody that are responsible for antigen binding. The hypervariable region generally comprises amino acid residues from a “complementarity determining region” or “CDR” (e.g., around about residues 24-34 (L1), 50-56 (L2) and 89-97 (L3) in the V_{L}, and around about 31-35 (H1), 50-65 (H2) and 95-102 (H3) in the V_{H} when numbered in accordance with the Kabat numbering system; Kabat et al., Sequences of Proteins of Immunological Interest, 5th Ed. Public Health Service, National Institutes of Health, Bethesda, Md. (1991)); and/or those residues from a “hypervariable loop” (e.g., residues 24-34 (L1), 50-56 (L2) and 89-97 (L3) in the V_{L}, and 26-32 (H1), 52-56 (H2) and 95-101 (H3) in the V_{H} when numbered in accordance with the Chothia numbering system; Chothia and Lesk, J. Mol. Biol. 196:901-917 (1987)); and/or those residues from a “hypervariable loop”/CDR (e.g., residues 27-38 (L1), 56-65 (L2) and 105-120 (L3) in the V_{L}, and 27-38 (H1), 56-65 (H2) and 105-120 (H3) in the V_{H} when numbered in accordance with the IMGT numbering system; Lefranc, M. P. et al. Nucl. Acids Res. 27:209-212 (1999), Ruiz, M. e al. Nucl. Acids Res. 28:219-221 (2000)).

The IMGT unique numbering has been defined to compare the variable domains whatever the antigen receptor, the chain type, or the species [Lefranc M.-P., Immunology Today 18, 509 (1997)/Lefranc M.-P., The Immunologist, 7, 132-136 (1999)/Lefranc, M.-P., Pommie, C., Ruiz, M., Giudicelli, V., Foulquier, E., Truong, L., Thouvenin-Contet, V. and Lefranc, Dev. Comp. Immunol., 27, 55-77 (2003)]. In the IMGT unique numbering, the conserved amino acids always have the same position, for instance cysteine 23 (Ist-CYS), tryptophan 41 (CONSERVED-TRP), hydrophobic amino acid 89, cysteine 104 (2nd-CYS), phenylalanine or tryptophan 118 (J-PHE or J-TRP). The IMGT unique numbering provides a standardized delimitation of the framework regions (FR1-IMGT: positions 1 to 26, FR2-IMGT: 39 to 55, FR3-IMGT: 66 to 104 and FR4-IMGT: 118 to 128) and of the complementarity determining regions: CDR1-IMGT: 27 to 38, CDR2-IMGT: 56 to 65 and CDR3-IMGT: 105 to 117. As gaps represent unoccupied positions, the CDR-IMGT lengths (shown between brackets and separated by dots, e.g. [8.8.13]) become crucial information. The IMGT unique numbering is used in 2D graphical representations, designated as IMGT Colliers de Perles (Ruiz, M. and Lefranc, M.-P., Immunogenetics, 53, 857-883 (2002)/Kaas, Q. and Lefranc, M.-P., Current Bioinformatics, 2, 21-30 (2007)), and in 3D structures in IMGT/3Dstructure-DB (Kaas, Q., Ruiz, M. and Lefranc, M.-P., T cell receptor and MHC structural data. Nucl. Acids. Res., 32, D208-D210 (2004)).

In some embodiments, CDRs are determined based on cross-species sequence variability (i.e., Kabat et al. Sequences of Proteins of Immunological Interest, (5th ed., 1991, National Institutes of Health, Bethesda Md.)). In some embodiments, CDRs are determined based on crystallographic studies of antigen-antibody complexes (Al-lazikani et al (1997) J. Molec. Biol. 273:927-948)). In addition, combinations of these two approaches can be used to determine CDRs. In some embodiments, the CDRs are determined based on AHo (Honegger and Pluckthun, J. Mol. Biol. 309(3):657-670; 2001). In some embodiments, CDRs are determined based on the IMGT system.

The term “human antibody” means an antibody produced by a human or an antibody having an amino acid sequence corresponding to an antibody produced by a human made using any technique known in the art. This definition of a human antibody includes intact or full-length antibodies, fragments thereof, and/or antibodies comprising at least one human heavy and/or light chain polypeptide such as, for example, an antibody comprising murine light chain and human heavy chain polypeptides.

A “neutralizing antibody” may inhibit the entry of HIV-1 virus for example SF162 and/or JR-CSF with a neutralization index >1.5 or >2.0. (Kostrikis L G et al. J Virol. 1996; 70(1): 445-458). By “broad and potent neutralizing antibodies” are meant antibodies that neutralize more than one HIV-1 virus species (from diverse clades and different strains within a clade) in a neutralization assay. A broad neutralizing antibody may neutralize at least 2, 3, 4, 5, 6, 7, 8, 9 or more different strains of HIV-1, the strains belonging to the same or different clades. A broad neutralizing antibody may neutralize multiple HIV-1 species belonging to at least 2, 3, 4, 5, or 6 different clades. In some embodiments, the concentration of the monoclonal antibody able to neutralize at 50% of the input virus in the neutralization assay can be less than about 50 □g/ml.

An “intact” antibody is one that comprises an antigen-binding site as well as a C_{L} and at least heavy chain constant domains, C_{H1}, C_{H2} and C_{H3}. The constant domains may be native sequence constant domains (e.g., human native sequence constant domains) or amino acid sequence variants thereof.

The term “chimeric antibodies” refers to antibodies wherein the amino acid sequence of the immunoglobulin molecule is derived from two or more species. Typically, the variable region of both light and heavy chains corresponds to the variable region of antibodies derived from one species of mammals (e.g. mouse, rat, rabbit, etc) with the desired specificity, affinity, and capability while the constant regions are homologous to the sequences in antibodies derived from another (usually human) to avoid eliciting an immune response in that species.

The antibodies herein also include antibodies in which a portion of the heavy and/or light chain is identical with or homologous to corresponding sequences in antibodies belonging to a particular antibody class or subclass, while the remainder of the chain(s) is identical with or homologous to corresponding sequences in antibodies derived from another species or belonging to another antibody class or subclass, as well as fragments of such antibodies.

In some embodiments, the antibody comprises variable region antigen-binding sequences derived from human antibodies (e.g., CDRs) and containing one or more sequences derived from a non-human antibody, e.g., an FR or C region sequence. In some embodiments, the antibody includes those comprising a human variable region antigen binding sequence of one antibody class or subclass and another sequence, e.g., FR or C region sequence, derived from another antibody class or subclass.

In some embodiments, chimeric antibodies may comprise residues that are not found in the recipient antibody or in the donor antibody. In some embodiments, modifications are made to further refine antibody performance. For further details, see Jones et al., Nature 321:522-525 (1986); Riechmann et al., Nature 332:323-329 (1988); and Presta, Curr. Op. Struct. Biol. 2:593-596 (1992).

The term “epitope” or “antigenic determinant” are used interchangeably herein and refer to that portion of an antigen capable of being recognized and specifically bound by a particular antibody. When the antigen is a polypeptide, epitopes can be formed both from contiguous amino acids and noncontiguous amino acids juxtaposed by tertiary folding of a protein. Epitopes formed from contiguous amino acids are typically retained upon protein denaturing, whereas epitopes formed by tertiary folding are typically lost upon protein denaturing. An epitope typically includes at least 3, and more usually, at least 5 or 8-10 amino acids in a unique spatial conformation.

“Binding affinity” generally refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Low-affinity antibodies generally bind antigen slowly and tend to dissociate readily, whereas high-affinity antibodies generally bind antigen faster and tend to remain bound longer.

The affinity or avidity of an antibody for an antigen can be determined experimentally using any suitable method well known in the art, e.g. flow cytometry, enzyme-linked immunoabsorbent assay (ELISA), or radioimmunoassay (RIA), or kinetics (e.g., BIACORE™ analysis). Direct binding assays as well as competitive binding assay formats can be readily employed. (See, for example, Berzofsky, et al., “Antibody-Antigen Interactions,” In Fundamental Immunology, Paul, W. E., Ed., Raven Press: New York, N.Y. (1984); Kuby, Janis Immunology, W.H. Freeman and Company: New York, N.Y. (1992); and methods described herein. The measured affinity of a particular antibody-antigen interaction can vary if measured under different conditions (e.g., salt concentration, pH, temperature). Thus, measurements of affinity and other antigen-binding parameters (e.g., KD or Kd, Kₒₙ, K_{off}) are made with standardized solutions of antibody and antigen, and a standardized buffer, as known in the art and such as the buffer described herein.

The phrase “substantially similar,” or “substantially the same”, as used herein, denotes a sufficiently high degree of similarity between two numeric values (generally one associated with an antibody of the invention and the other associated with a reference/comparator antibody) such that one of skill in the art would consider the difference between the two values to be of little or no biological and/or statistical significance within the context of the biological characteristics measured by said values (e.g., Kd values). The difference between said two values is less than about 500%, less than about 40%, less than about 300%, less than about 200%, or less than about 10% as a function of the value for the reference/comparator antibody.

A polypeptide, antibody, polynucleotide, vector, cell, or composition which is “isolated” is a polypeptide, antibody, polynucleotide, vector, cell, or composition which is in a form not found in nature. Isolated polypeptides, antibodies, polynucleotides, vectors, cell or compositions include those which have been purified to a degree that they are no longer in a form in which they are found in nature. In some embodiments, an antibody, polynucleotide, vector, cell, or composition which is isolated is substantially pure.

An “isolated nucleic acid” is a nucleic acid that is substantially separated from other genome DNA sequences as well as proteins or complexes such as ribosomes and polymerases, which naturally accompany a native sequence. The term embraces a nucleic acid sequence that has been removed from its naturally occurring environment, and includes recombinant or cloned DNA isolates and chemically synthesized analogues or analogues biologically synthesized by heterologous systems. A substantially pure nucleic acid includes isolated forms of the nucleic acid. Of course, this refers to the nucleic acid as originally isolated and does not exclude genes or sequences later added to the isolated nucleic acid by the hand of man.

An “isolated polypeptide” is one that has been identified and separated and/or recovered from a component of its natural environment. In preferred embodiments, the isolated polypeptide will be purified (1) to greater than 95% by weight of polypeptide as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or non-reducing conditions using Coomassie blue or, preferably, silver stain. Isolated polypeptide includes the polypeptide in situ within recombinant cells since at least one component of the polypeptide's natural environment will not be present.

A “native sequence” polynucleotide is one that has the same nucleotide sequence as a polynucleotide derived from nature. A “native sequence” polypeptide is one that has the same amino acid sequence as a polypeptide (e.g., antibody) derived from nature (e.g., from any species). Such native sequence polynucleotides and polypeptides can be isolated from nature. A polynucleotide “variant,” as the term is used herein, is a polynucleotide that typically differs from a polynucleotide specifically disclosed herein in one or more substitutions, deletions, additions and/or insertions. Such variants may be naturally occurring or may be synthetically generated, for example, by modifying one or more of the polynucleotide sequences of the invention and evaluating one or more biological activities of the encoded polypeptide as described herein and/or using any of a number of techniques well known in the art.

A polypeptide “variant,” as the term is used herein, is a polypeptide that typically differs from a polypeptide specifically disclosed herein in one or more substitutions, deletions, additions and/or insertions. Such variants may be naturally occurring or may be synthetically generated, for example, by modifying one or more of the above polypeptide sequences of the invention and evaluating one or more biological activities of the polypeptide as described herein and/or using any of a number of techniques well known in the art. or can be produced by recombinant or synthetic means.

As used herein, “substantially pure” refers to material which is at least 50% pure (i.e., free from contaminants), at least 90% pure, at least 95% pure, at least 98% pure, or at least 99% pure.

The term “subject” refers to any animal (e.g., a mammal), including, but not limited to humans, non-human primates, rodents, and the like, which is to be the recipient of a particular treatment. Typically, the terms “subject” and “patient” are used interchangeably herein in reference to a human subject.

Administration “in combination with” one or more further therapeutic agents includes simultaneous (concurrent) and consecutive administration in any order.

The term “pharmaceutical formulation” refers to a preparation which is in such form as to permit the biological activity of the active ingredient to be effective, and which contains no additional components which are unacceptably toxic to a subject to which the formulation would be administered. Such formulation can be sterile.

An “effective amount” of an antibody as disclosed herein is an amount sufficient to carry out a specifically stated purpose. An “effective amount” can be determined empirically and in a routine manner, in relation to the stated purpose.

The term “therapeutically effective amount” refers to an amount of an antibody or other drug effective to “treat” or prevent a disease or disorder in a subject or mammal.

Terms such as “treating” or “treatment” or “to treat” or “alleviating” or “to alleviate” refer to both 1) therapeutic measures that cure, slow down, lessen symptoms of, and/or halt progression of a diagnosed pathologic condition or disorder and 2) prophylactic or preventative measures that prevent and/or slow the development of a targeted pathologic condition or disorder. Thus, those in need of treatment include those already with the disorder; those prone to have the disorder; and those in whom the disorder is to be prevented.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase. A polynucleotide can comprise modified nucleotides, such as methylated nucleotides and their analogs. If present, modification to the nucleotide structure can be imparted before or after assembly of the polymer. The sequence of nucleotides can be interrupted by non-nucleotide components. A polynucleotide can be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, cabamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars can be replaced, for example, by phosphonate groups, phosphate groups, protected by standard protecting groups, or activated to prepare additional linkages to additional nucleotides, or can be conjugated to solid supports. The 5′ and 3′ terminal OH can be phosphorylated or substituted with amines or organic capping group moieties of from 1 to 20 carbon atoms. Other hydroxyls can also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, alpha.-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages can be replaced by alternative linking groups. These alternative linking groups include, but are not limited to, embodiments wherein phosphate is replaced by P(O)S (“thioate”), P(S)S (“dithioate”), “(O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

The terms “polypeptide,” “peptide,” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this invention are based upon antibodies, in certain embodiments, the polypeptides can occur as single chains or associated chains.

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software are known in the art that can be used to obtain alignments of amino acid or nucleotide sequences. One such non-limiting example of a sequence alignment algorithm is the algorithm described in Karlin et al, 1990, Proc. Natl. Acad. Sci., 87:2264-2268, as modified in Karlin et al., 1993, Proc. Natl. Acad. Sci., 90:5873-5877, and incorporated into the NBLAST and XBLAST programs (Altschul et al., 1991, Nucleic Acids Res., 25:3389-3402). In certain embodiments, Gapped BLAST can be used as described in Altschul et al., 1997, Nucleic Acids Res. 25:3389-3402. BLAST-2, WU-BLAST-2 (Altschul et al., 1996, Methods in Enzymology, 266:460-480), ALIGN, ALIGN-2 (Genentech, South San Francisco, Calif.) or Megalign (DNASTAR) are additional publicly available software programs that can be used to align sequences. In certain embodiments, the percent identity between two nucleotide sequences is determined using the GAP program in GCG software (e.g., using a NWSgapdna.CMP matrix and a gap weight of 40, 50, 60, 70, or 90 and a length weight of 1, 2, 3, 4, 5, or 6). In certain alternative embodiments, the GAP program in the GCG software package, which incorporates the algorithm of Needleman and Wunsch (J. Mol. Biol. (48):444-453 (1970)) can be used to determine the percent identity between two amino acid sequences (e.g., using either a Blossum 62 matrix or a PAM250 matrix, and a gap weight of 16, 14, 12, 10, 8, 6, or 4 and a length weight of 1, 2, 3, 4, 5). Alternatively, in certain embodiments, the percent identity between nucleotide or amino acid sequences is determined using the algorithm of Myers and Miller (CABIOS, 4:11-17 (1989)). For example, the percent identity can be determined using the ALIGN program (version 2.0) and using a PAM120 with residue table, a gap length penalty of 12 and a gap penalty of 4. Appropriate parameters for maximal alignment by particular alignment software can be determined by one skilled in the art. In certain embodiments, the default parameters of the alignment software are used. In certain embodiments, the percentage identity “X” of a first amino acid sequence to a second sequence amino acid is calculated as 100.times.(Y/Z), where Y is the number of amino acid residues scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be longer than the percent identity of the second sequence to the first sequence.

As a non-limiting example, whether any particular polynucleotide has a certain percentage sequence identity (e.g., is at least 80% identical, at least 85% identical, at least 90% identical, and in some embodiments, at least 95%, 96%, 97%, 98%, or 99% identical) to a reference sequence can, in certain embodiments, be determined using the Bestfit program (Wisconsin Sequence Analysis Package, Version 8 for Unix, Genetics Computer Group, University Research Park, 575 Science Drive, Madison, Wis. 53711). Bestfit uses the local homology algorithm of Smith and Waterman. Advances in Applied Mathematics 2: 482 489 (1981), to find the best segment of homology between two sequences. When using Bestfit or any other sequence alignment program to determine whether a particular sequence is, for instance, 95% identical to a reference sequence according to the present invention, the parameters are set such that the percentage of identity is calculated over the full length of the reference nucleotide sequence and that gaps in homology of up to 5% of the total number of nucleotides in the reference sequence are allowed.

In some embodiments, two nucleic acids or polypeptides of the invention are substantially identical, meaning they have at least 70%, at least 75%, at least 80%, at least 85%, at least 90%, and in some embodiments at least 95%, 96%, 97%, 98%, 99% nucleotide or amino acid residue identity, when compared and aligned for maximum correspondence, as measured using a sequence comparison algorithm or by visual inspection. In certain embodiments, identity exists over a region of the sequences that is at least about 10, about 20, about 40-60 residues in length or any integral value therebetween, or over a longer region than 60-80 residues, at least about 90-100 residues, or the sequences are substantially identical over the full length of the sequences being compared, such as the coding region of a nucleotide sequence for example.

A “conservative amino acid substitution” is one in which one amino acid residue is replaced with another amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. In certain embodiments, conservative substitutions in the sequences of the polypeptides and antibodies of the invention do not abrogate the binding of the polypeptide or antibody containing the amino acid sequence, to the antigen(s), i.e., the gp120 to which the polypeptide or antibody binds. Methods of identifying nucleotide and amino acid conservative substitutions which do not eliminate antigen binding are well-known in the art (see, e.g., Brummell et al., Biochem. 32: 1180-1187 (1993); Kobayashi et al. Protein Eng. 12(10):879-884 (1999); and Burks et al. Proc. Natl. Acad. Sci. USA 94:412-417 (1997)).

### Liposomal Pharmaceutical Formulations

In one embodiment, the disclosure provides a liposomal pharmaceutical formulation for subcutaneous or intravenous administration of antibodies in a subject, comprising
a. one or more phospholipids;
b. one or more antibodies; and
c. a pharmaceutically acceptable vehicle,
   wherein the one or more phospholipids form liposomes that encapsulate the one or more antibodies, wherein the liposomes have a polydispersity index (PDI) below 0.2 and at least 90% of the liposomes have a size less than 125 nm.

In some embodiments, the liposomes further comprise an effective amount of cholesterol or a derivative thereof. In some embodiments, the liposomes comprise cholesterol or a derivative thereof selected from the group consisting of cholesterol, cholesteryl oleate, and cholesterol hemisuccinate (CHEMS), and combinations thereof. In some embodiments, the liposomes comprise cholesterol.

The liposomal pharmaceutical formulation can be relatively homogenous. A polydispersity index can be used to indicate the homogeneity of a liposomal pharmaceutical formulation, e.g., the particle size distribution of the liposomes. A small (e.g., less than 0.3) polydispersity index generally indicates a narrow particle size distribution. A liposomal pharmaceutical formulation can have a polydispersity index from about 0 to less than 0.20, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, or 0.19. In some embodiments, the polydispersity index of a nanoparticle composition disclosed herein can be from about 0.10 to less than 0.20. In some embodiments, the liposomes have a polydispersity index below 0.19, 0.18, 0.17, 0.16, 0.15, 0.14, 0.13, 0.12, 0.11, or 0.10.

In some embodiments, at least 90% of the liposomes have a size less than 125 nm. In some embodiments, at least 90% of the liposomes have a size less than 120 nm. In some embodiments, at least 90% of the liposomes have a size less than 115 nm. In some embodiments, at least 90% of the liposomes have a size less than 110 nm. In some embodiments, at least 90% of the liposomes have a size less than 105 nm. In some embodiments, at least 90% of the liposomes have a size less than 100 nm. In some embodiments, at least 90% of the liposomes have a size less than 95 nm. In some embodiments, at least 90% of the liposomes have a size less than 90 nm.

In some embodiments, at least 90% of the liposomes have a size ranging from 50 nm to 100 nm. In some embodiments, at least 90% of the liposomes have a size ranging from 75 nm to 100 nm. In some embodiments, at least 80% of the liposomes have a size ranging from 92 nm+/−3.6 nm. In some embodiments, at least 80% of the liposomes have a size ranging from 85 nm+/−5.8 nm.

The zeta potential of the liposomes can be used to indicate the electro kinetic potential of the composition. For example, the zeta potential can describe the surface charge of a liposomal formulation. In some embodiments, the zeta potential of the liposomes can be from about 0 mV to about −100 mV, from about 0 mV to about −90 mV, from about 0 mV to about −80 mV, from about 0 mV to about −70 mV, from about 0 mV to about −60 mV, from about 0 mV to about −50 mV, from about 0 mV to about −40 mV, from about 0 mV to about −30 mV, from about 0 mV to about −20 mV, from about 0 mV to about −10 mV, from about −10 mV to about −100 mV, from about −10 mV to about −90 mV, from about −10 mV to about −80 mV, from about −10 mV to about −70 mV, from about −10 mV to about −60 mV, from about −10 mV to about −50 mV, from about −10 mV to about −40 mV, from about −10 mV to about −30 mV, from about −10 mV to about −20 mV, from about −20 mV to about −100 mV, from about −20 mV to about −90 mV, from about −20 mV to about −80 mV, from about −20 mV to about −70 mV, from about −20 mV to about −60 mV, from about −20 mV to about −50 mV, from about −20 mV to about −40 mV, from about −20 mV to about −30 mV, from about −30 mV to about −100 mV, from about −30 mV to about −90 mV, from about −30 mV to about −80 mV, from about −30 mV to about −70 mV, from about −30 mV to about −60 mV, from about −30 mV to about −50 mV, from about −30 mV to about −40 mV, from about −40 mV to about −100 mV, from about −40 mV to about −90 mV, from about −40 mV to about −80 mV, from about −40 mV to about −70 mV, from about −40 mV to about −60 mV, and from about −40 mV to about −50 mV. In some embodiments, the zeta potential of the liposomes can be from about −10 mV to about −50 mV, from about −15 mV to about −45 mV, from about −20 mV to about −40 mV, and from about −25 mV to about −35 mV. In some embodiments, the zeta potential of the liposomes can be about −10 mV, about −20 mV, about −30 mV, about −40 mV, about −50 mV, about −60 mV, about −70 mV, about −80 mV, about −90 mV, and about −100 mV.

In some embodiments, the zeta potential of the liposomes is from −35 mV to −50 mV. In some embodiments, the zeta potential of the liposomes is from −42 mV+/−3.7 mV. In some embodiments, the zeta potential of the liposomes is from −10 mV to −20 mV. In some embodiments, the zeta potential of the liposomes is from −14.5 mV+/−2.7 mV.

The phospholipids that can be used are not particularly limiting. In some embodiments, the one or more phospholipids are selected from hydrogenated soy phosphatidylcholine (HSPC), 1,2-Distearoyl-sn-glycero-3-phosphoglycerol (DSPG), 1,2-distearoyl-sn-glycero-3-phosphoethanolamine (DSPE) and combinations thereof. In some embodiments, DSPG comprises 1,2-distearoyl-sn-glycero-3-phospho-(1′-rac-glycerol) (sodium salt). In some embodiments, the phospholipids are selected from phosphatidylcholine (PC), phosphatidylethanolamine (PE), phosphatidylserine (PS), phosphatidylglycerol (PG), phosphatidylinositol (PI), sphingomyelin (SM), 1,2-dipalmitoyl-sn-glycero-3-phosphocholine (DPPC), 1,2-distearoyl-sn-glycero-3-phosphocholine (DSPC), 1,2-dioleoyl-sn-glycero-3-phosphocholine (DOPC), and 1,2-dimyristoyl-sn-glycero-3-phosphocholine (DMPC).

In some embodiments, one or more phospholipids is pegylated. In some embodiments, the DSPE is pegylated. In some embodiments, the pegylated DSPE is 1,2-distearoyl-sn-glycero-3-phosphoethanolamine-N-[methoxy(polyethylene glycol)-2000](DSPE-mPEG2000).

The term “encapsulation efficiency” of an antibody describes the amount of the antibody that is encapsulated by the liposomes after preparation, relative to the initial amount of antibody provided. As used herein, “encapsulation” can refer to complete, substantial, or partial enclosure, confinement, surrounding, or encasement.

In some embodiments, encapsulation efficiency is desirably high (e.g., close to 100%). The encapsulation efficiency can be measured, for example, by comparing the amount of the antibody in a solution containing the liposomes before and after breaking up the liposomes with one or more organic solvents or detergents.

For the liposomal compositions described herein, the encapsulation efficiency of the antibody can be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency can be at least 55%. In certain embodiments, the encapsulation efficiency can be at least 60%.

In some embodiments, the liposome comprises a combination of phospholipids and a cholesterol or derivative thereof. In some embodiments, the liposome comprises HSPC, DSPG, cholesterol and DSPE-mPEG2000. In some embodiments, the liposome comprises HSPC, cholesterol and DSPG.

The molar ratio of the various phospholipids and cholesterol is not necessarily limiting. For example, in some embodiments, the liposome comprises HSPC, cholesterol, DSPE-mPEG2000 and DSPG wherein the molar ratio of HSPC:cholesterol:DSPE-mPEG2000:DSPG is from about 40-70:25-45:2-15:1-20. In some embodiments, the molar ratio of HSPC:cholesterol:DSPE-mPEG2000:DSPG is about 50:35:5:10.

In some embodiments, the liposome comprises HSPC, cholesterol, and DSPG wherein the molar ratio of HSPC:cholesterol:DSPG is about 40-80:10-30:10-30.

In some embodiments, the molar ratio of HSPC:cholesterol:DSPG is about 50-60:10-30:10-30. In some embodiments, the molar ratio of HSPC:cholesterol:DSPG is about 60:20:20.

The formulations comprise a pharmaceutically acceptable vehicle (e.g. carrier, excipient) (Remington, The Science and Practice of Pharmacy 20th Edition Mack Publishing, 2000). Suitable pharmaceutically acceptable vehicles include, but are not limited to, water, saline, nontoxic buffers such as phosphate, citrate, and other organic acids; salts such as sodium chloride; antioxidants including ascorbic acid and methionine; preservatives (e.g. octadecyldimethylbenzyl ammonium chloride; hexamethonium chloride; benzalkonium chloride; benzethonium chloride; phenol, butyl or benzyl alcohol; alkyl parabens, such as methyl or propyl paraben; catechol; resorcinol; cyclohexanol; 3-pentanol; and m-cresol); low molecular weight polypeptides (e.g. less than about 10 amino acid residues); proteins such as serum albumin, gelatin, or immunoglobulins; hydrophilic polymers such as polyvinylpyrrolidone; amino acids such as glycine, glutamine, asparagine, histidine, arginine, or lysine; carbohydrates such as monosaccharides, disaccharides, glucose, mannose, or dextrins; chelating agents such as EDTA; sugars such as sucrose, mannitol, trehalose or sorbitol; salt-forming counter-ions such as sodium; metal complexes (e.g. Zn-protein complexes); and non-ionic surfactants such as TWEEN or polyethylene glycol (PEG).

In some embodiments, the liposomes are embedded or encapsulated in an injectable matrix. In some embodiments, the liposomes are embedded in microspheres, a silica matrix, or a hydrogel matrix. In some embodiments, the microspheres are embedded in a hydrogel, such as gelatin.

In some embodiments, the microsphere comprises a biodegradable and biocompatible copolymer. In some embodiments, the microsphere comprises poly(lactic-co-glycolic acid) (PGLA). In some embodiments, the PGLA is PLGA (75:25). In some embodiments, the polymer is selected from PGLA, polycaprolactone (PCL) and polylactic acid (PLA).

In some embodiments, the liposomes are embedded in an injectable hydrogel matrix. In some embodiments, the injectable hydrogel matrix comprises poly(vinyl alcohol) (PVA). In some embodiments, the injectable hydrogel matrix comprises a blend of PVA and gelatin. In some embodiments, the injectable hydrogel matrix comprises one or more of alginate, PVA, chitosan, hyaluronic acid (HA), or dextran-based hydrogel.

In some embodiments, the injectable matrix is chemically or physically crosslinked. In some embodiments, the injectable matrix is crosslinked using genipin.

The antibody to be formulated in liposomal pharmaceutical formulation is not limiting. In some embodiments, the antibody is a polyclonal antibody. In some embodiments, the antibody is a monoclonal antibody.

In some embodiments, the antibody is an anti-HIV antibody. In some embodiments, the anti-HIV antibody is a broadly neutralizing and potent antibody against HIV. In some embodiments, the antibody is antibody PGT121.

In some embodiments, the antibody is an anti-HIV antibody described in U.S. Pat. Appl. Pub. Nos: 2020/0239551 A1 and 2023/0242628 A1 and WO 2024/123897, which are incorporated by reference in their entireties. The foregoing published patent applications describe the N49P series of antibodies, which comprise natural antibodies as well as engineered variants of the natural antibodies.

In some embodiments, the antibody is selected from the natural N49P series antibody sequences 1-38 as shown in Table 1 below. In some embodiments, the antibody is derived from a N49P series antibody, wherein the N49P series antibody is modified whereby a part or all of the framework 3 region of the heavy chain is replaced with an amino acid sequence selected from the group consisting of QLSQDPDDPDWG (SEQ ID NO:541) and LFSQDLYYPDRG (SEQ ID NO:542). In some embodiments, the framework 3 region in the N49P series antibody is already deleted or missing, and in those cases either SEQ ID NO: 541 or 542 is inserted therein in the framework 3 region.

In some embodiments, the antibody comprises variants of these natural antibodies. In some embodiments, the variants are selected from antibodies N49P6, N49P6.2, N49P7, N49P7.1, N49P7A, N49P7S, N49P7F, N49P7Y, N49P7.54TY, N49P7-LS1, N49P7-LS2, N49P7/6L, N49P7/11L, R49P7,N49P7.2, N49P11, N49P18, N49P18.2, N49P18.1, N49P19, N49P37, N49P38, N49P38.1, N49P55, N49P56, N49P57, N49P58, N49P59, N49P73, N49P74, N49P75, N49P9, N49P9.1, N49P9.2, N49P9i7, N49P22, N49P23, N49P9.3, N49P9.4, N49P9.6, N49P9.6-54W, N49P9.6-54F, N49P51, N49P52, N49P53, N49P54, N49P60, N49P61, N49P62, N49P63, N49P64, N49P65, N49P66, N49P67, N49P68, N49P69, N49P70, N49P71, and N49P72. These variants are described in Table 3, below.

In some embodiments, the antibody is antibody N49P7-FR or an antigen binding fragment thereof. The amino acid sequence of the heavy chain of N49P7-FR is SEQ ID NO:501 and the nucleotide sequence is SEQ ID NO:502. The amino acid sequence of the light chain of N49P7-FR is SEQ ID NO:503 and the nucleotide sequence is SEQ ID NO:504.

In some embodiments, the antibody is antibody N49P9-FR or an antigen binding fragment thereof. The amino acid sequence of the heavy chain of N49P9-FR is SEQ ID NO:505 and the nucleotide sequence is SEQ ID NO:506. The amino acid sequence of the light chain of N49P9-FR is SEQ ID NO:295 and the nucleotide sequence is SEQ ID NO:296.

In some embodiments, the antibody is antibody N49P9.3-FR or an antigen binding fragment thereof. The amino acid sequence of the heavy chain of N49P9.3-FR is SEQ ID NO:507 and the nucleotide sequence is SEQ ID NO:508. The amino acid sequence of the light chain of N49P9.3-FR is SEQ ID NO:327 and the nucleotide sequence is SEQ ID NO:328.

In some embodiments, the antibody is antibody N49P9.6-FR or an antigen binding fragment thereof. The amino acid sequence of the heavy chain of N49P9.6-FR is SEQ ID NO:509 and the nucleotide sequence is SEQ ID NO:510. The amino acid sequence of the light chain of N49P9.6-FR is SEQ ID NO:511 and the nucleotide sequence is SEQ ID NO:512.

In some embodiments, the antibody is antibody N49P9.6-FR-54W or an antigen binding fragment thereof. The amino acid sequence of the heavy chain of N49P9.6-FR-54W is SEQ ID NO:513 and the nucleotide sequence is SEQ ID NO:514. The amino acid sequence of the light chain of N49P9.6-FR-54W is SEQ ID NO:515 and the nucleotide sequence is SEQ ID NO:516.

In some embodiments, the antibody is antibody N49P9.6-FR-54F or an antigen binding fragment thereof. The amino acid sequence of the heavy chain of N49P9.6-FR-54F is SEQ ID NO:517 and the nucleotide sequence is SEQ ID NO:518. The amino acid sequence of the light chain of N49P9.6-FR-54F is SEQ ID NO:519 and the nucleotide sequence is SEQ ID NO:520.

In some embodiments, the antibody is antibody N49P9.6-FR3-06 or an antigen binding fragment thereof. The amino acid sequence of the heavy chain of N49P9.6-FR3-06 is SEQ ID NO:521 and the nucleotide sequence is SEQ ID NO:522. The amino acid sequence of the light chain of N49P9.6-FR3-06 is SEQ ID NO:523 and the nucleotide sequence is SEQ ID NO:524.

In some embodiments, the antibody is antibody N49P9.6-FR1-D or an antigen binding fragment thereof. The amino acid sequence of the heavy chain of N49P9.6-FR1-D is SEQ ID NO:525 and the nucleotide sequence is SEQ ID NO:526. The amino acid sequence of the light chain of N49P9.6-FR1-D is SEQ ID NO:527 and the nucleotide sequence is SEQ ID NO:528.

In some embodiments, the antibody is antibody N49P9.6-FR1-D-I or an antigen binding fragment thereof. The amino acid sequence of the heavy chain of N49P9.6-FR1-D-I is SEQ ID NO:529 and the nucleotide sequence is SEQ ID NO:530. The amino acid sequence of the light chain of N49P9.6-FR1-D-I is SEQ ID NO:531 and the nucleotide sequence is SEQ ID NO:532.

In some embodiments, the antibody is antibody N49P9.6-FR-LS or an antigen binding fragment thereof. The amino acid sequence of the heavy chain of N49P9.6-FR-LS is SEQ ID NO:533 and the nucleotide sequence is SEQ ID NO:534. The amino acid sequence of the light chain of N49P9.6-FR-LS is SEQ ID NO:535 and the nucleotide sequence is SEQ ID NO:536.

In some embodiments, the antibody is antibody N49P9.6-FR-YTE or an antigen binding fragment thereof. The amino acid sequence of the heavy chain of N49P9.6-FR-YTE is SEQ ID NO:537 and the nucleotide sequence is SEQ ID NO:538. The amino acid sequence of the light chain of N49P9.6-FR-YTE is SEQ ID NO:539 and the nucleotide sequence is SEQ ID NO:540.

In some embodiments, the antibody comprises the VH and VL regions of antibody N49P7-FR, N49P9-FR, N49P9.3-FR, N49P9.6-FR, N49P9.6-FR-54W, N49P9.6-FR-54F, N49P9.6-FR3-06, N49P9.6-FR1-D, N49P9.6-FR1-D-I, N49P9.6, N49P9.6-54W, N49P9.6-54F, N49P9.6-LS, N49P9.6-YTE, N49P9.6-FR-LS, or N49P9.6-FR-YTE as shown below.

In some embodiments, the antibody comprises the heavy chain CDR and light chain CDR sequences of the antibodies N49P7-FR, N49P9-FR, N49P9.3-FR, N49P9.6-FR, N49P9.6-FR-54W, N49P9.6-FR-54F, N49P9.6-FR3-06, N49P9.6-FR1-D, N49P9.6-FR1-D-I, N49P9.6, N49P9.6-54W, N49P9.6-54F, N49P9.6-LS, N49P9.6-YTE, N49P9.6-FR-LS, or N49P9.6-FR-YTE as shown below. In some embodiments, the antibody is further modified and comprises a framework 3 region of the heavy chain comprising an amino acid sequence selected from QLSQDPDDPDWG (SEQ ID NO:541) and LFSQDLYYPDRG (SEQ ID NO:542).

The naturally occurring N49P series antibodies are shown below in Table 1 (antibody sequences 1-38), and the related variants are indicated.

TABLE 1
Amino acid sequence of natural antibodies. Heavy chain is shown first (above dotted
line), followed by the light chain (below dotted line). Sequences of CDR H1, CDR
H2, CDR H3, CDR L1, CDR L2, and CDR L3 are in bold. Note that there is a
predicted blank CDR in the light chain for Sequences 25, 34 and 36.

| Natural antibody | Related variants | Amino Acid sequence |
|---|---|---|
| Sequence 1 | N49P6, N49P6.2 | 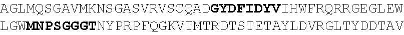 |
| | | YYCVRDRANGSGRRRFESVNWFLDLWGRGTQITVVSPSTKGPSVFP |
| | | LAPSSKSTSGGTAALGCLVKDYFPEPVTVSWNSGALTSGVHTFPAV |
| | | LQSSGLYSLSSVVTVPSSSLGTQTYICNVNHKPSNTKVDKKVEPKS |
| | | CDKTHTCPPCPAPELLGGPSVFLFPPKPKDTLMISRTPEVTCVVVD |
| | | VSHEDPEVKENWYVDGVEVHNAKTKPREEQYNSTYRVVSVLTVLHQ |
| | | DWLNGKEYKCKVSNKALPAPIEKTISKAKGQPREPQVYTLPPSRDE |
| | | LTKNQVSLTCLVKGFYPSDIAVEWESNGQPENNYKTTPPVLDSDGS |
| | | FFLYSKLTVDKSRWQQGNVFSCSVMHEALHNHYTQKSLSLSPGK |
| | | SEQ ID NO: 1 |
| | | 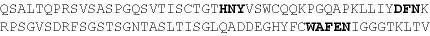 |
| | | LRQPKAAPSVTLFPPSSEELQANKATLVCLISDFYPGAVTVAWKAD |
| | | SSPVKAGVETTTPSKQSNNKYAASSYLSLTPEQWKSHRSYSCQVTH |
| | | EGSTVEKTVAPTECS SEQ ID NO: 2 |
| Sequence 2 | N49P7, N49P7.1 N49P7A | 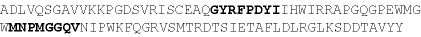 |
| | N49P7S | CVRDRSNGSGKRFESSNWFLDLWGRGTAVTIQSSSTKGPSVFPLAPSS |
| | N49P7F | KSTSGGTAALGCLVKDYFPEPVTVSWNSGALTSGVHTFPAVLQSSGLY |
| | N49P7Y | SLSSVVTVPSSSLGTQTYICNVNHKPSNTKVDKKVEPKSCDKTHTCPP |
| | N49P7.54TY | CPAPELLGGPSVFLFPPKPKDTLMISRTPEVTCVVVDVSHEDPEVKFN |
| | N49P7-LS1 | WYVDGVEVHNAKTKPREEQYNSTYRVVSVLTVLHQDWLNGKEYKCKVS |
| | N49P7-LS2 | NKALPAPIEKTISKAKGQPREPQVYTLPPSRDELTKNQVSLTCLVKGF |
| | N49P7/6L | YPSDIAVEWESNGQPENNYKTTPPVLDSDGSFFLYSKLTVDKSRWQQG |
| | N49P7/11L | NVFSCSVMHEALHNHYTQKSLSLSPGK |
| | R49P7 | SEQ ID NO: 3 |
| | N49P7-FR | 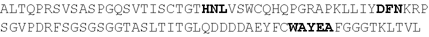 |
| | | RQPKAAPSVTLFPPSSEELQANKATLVCLISDFYPGAVTVAWKADSSPV |
| | | KAGVETTTPSKQSNNKYAASSYLSLTPEQWKSHRSYSCQVTHEGSTVEK |
| | | TVAPTECS |
| | | SEQ ID NO: 4 |
| Sequence 3 | N49P7.2 | 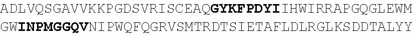 |
| | | CVRDRSNGSGRRFESSNWFLDLWGRGTAVTVHSPSKSTSGGTAALGC |
| | | LVKDYFPEPVTVSWNSGALTSGVHTFPAVLQSSGLYSLSSVVTVPSS |
| | | SLGTQTYICNVNHKPSNTKVDKKVEPKSCDKTHTCPPCPAPELLGGP |
| | | SVFLFPPKPKDTLMISRTPEVTCVVVDVSHEDPEVKFNWYVDGVEVH |
| | | NAKTKPREEQYNSTYRVVSVLTVLHQDWLNGKEYKCKVSNKALPAPI |
| | | EKTISKAKGQPREPQVYTLPPSRDELTKNQVSLTCLVKGFYPSDIAV |
| | | EWESNGQPENNYKTTPPVLDSDGSFFLYSKLTVDKSRWQQGNVFSCS |
| | | VMHEALHNHYTQKSLSLSPGK |
| | | SEQ ID NO: 5 |
| | | 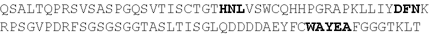 |
| | | VLRQPKAAPSVTLFPPSSEELQANKATLVCLISDFYPGAVTVAWKADSS |
| | | PVKAGVETTTPSKQSNNKYAASSYLSLTPEQWKSHRSYSCQVTHEGSTV |
| | | EKTVAPTECS |
| | | SEQ ID NO: 6 |
| Sequence 4 | N49P11 | 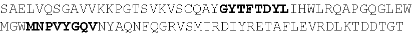 |
| | | YYCVRDTGDGSRRHFDSINWFLDLWGRGTWIRVAPASTKGPSVFPLA |
| | | PSSKSTSGGTAALGCLVKDYFPEPVTVSWNSGALTSGVHTFPAVLQS |
| | | SGLYSLSSVVTVPSSSLGTQTYICNVNHKPSNTKVDKKVEPKSCDKT |
| | | HTCPPCPAPELLGGPSVFLFPPKPKDTLMISRTPEVTCVVVDVSHED |
| | | PEVKFNWYVDGVEVHNAKTKPREEQYNSTYRVVSVLTVLHQDWLNGK |
| | | EYKCKVSNKALPAPIEKTISKAKGQPREPQVYTLPPSRDELTKNQVS |
| | | LTCLVKGFYPSDIAVEWESNGQPENNYKTTPPVLDSDGSFFLYSKLT |
| | | VDKSRWQQGNVFSCSVMHEALHNHYTQKSLSLSPGK |
| | | SEQ ID NO: 7 |
| | | 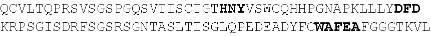 |
| | | VLRQPKAAPSVTLFPPSSEELQANKATLVCLISDFYPGAVTVAWKADSS |
| | | PVKAGVETTTPSKQSNNKYAASSYLSLTPEQWKSHRSYSCQVTHEGSTV |
| | | EKTVAPTECS |
| | | SEQ ID NO: 8 |
| Sequence 5 | N49P18 N49P18.2 | 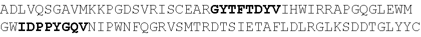 |
| | | VRDRSNGWGKRFESSNWFLDLWGRGTVVTVHSPSTKGPSVFPLAPSSK |
| | | STSGGTAALGCLVKDYFPEPVTVSWNSGALTSGVHTFPAVLQSSGLYS |
| | | LSSVVTVPSSSLGTQTYICNVNHKPSNTKVDKKVEPKSCDKTHTCPPC |
| | | PAPELLGGPSVFLFPPKPKDTLMISRTPEVTCVVVDVSHEDPEVKFNW |
| | | YVDGVEVHNAKTKPREEQYNSTYRVVSVLTVLHQDWLNGKEYKCKVSN |
| | | KALPAPIEKTISKAKGQPREPQVYTLPPSRDELTKNQVSLTCLVKGFY |
| | | PSDIAVEWESNGQPENNYKTTPPVLDSDGSFFLYSKLTVDKSRWQQGN |
| | | VFSCSVMHEALHNHYTQKSLSLSPGK |
| | | SEQ ID NO: 9 |
| | | 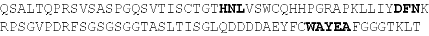 |
| | | VLRQPKAAPSVTLFPPSSEELQANKATLVCLISDFYPGAVTVAWKADSS |
| | | PVKAGVETTTPSKQSNNKYAASSYLSLTPEQWKSHRSYSCQVTHEGSTV |
| | | EKTVAPTECS |
| | | SEQ ID NO: 10 |
| Sequence 6 | N49P18.1 | 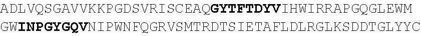 |
| | | VRDRSNGWGKRFESSNWFLDLWGRGTVVTVHSPSTKGPSVFPLAPSSK |
| | | STSGGTAALGCLVKDYFPEPVTVSWNSGALTSGVHTFPAVLQSSGLYS |
| | | LSSVVTVPSSSLGTQTYICNVNHKPSNTKVDKKVEPKSCDKTHTCPPC |
| | | PAPELLGGPSVFLFPPKPKDTLMISRTPEVTCVVVDVSHEDPEVKFNW |
| | | YVDGVEVHNAKTKPREEQYNSTYRVVSVLTVLHQDWLNGKEYKCKVSN |
| | | KALPAPIEKTISKAKGQPREPQVYTLPPSRDELTKNQVSLTCLVKGFY |
| | | PSDIAVEWESNGQPENNYKTTPPVLDSDGSFFLYSKLTVDKSRWQQGN |
| | | VFSCSVMHEALHNHYTQKSLSLSPGK |
| | | SEQ ID NO: 11 |
| | | 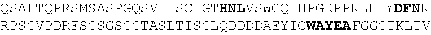 |
| | | LRQPKAAPSVTLFPPSSEELQANKATLVCLISDFYPGAVTVAWKADSSP |
| | | VKAGVETTTPSKQSNNKYAASSYLSLTPEQWKSHRSYSCQVTHEGSTVE |
| | | KTVAPTECS |
| | | SEQ ID NO: 12 |
| Sequence 7 | N49P19 | 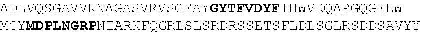 |
| | | CVRDKSNGSGRRFDSSNWFLDLWGRGTRVSIFSASTKGPSVFPLAPSS |
| | | KSTSGGTAALGCLVKDYFPEPVTVSWNSGALTSGVHTFPAVLQSSGLY |
| | | SLSSVVTVPSSSLGTQTYICNVNHKPSNTKVDKKVEPKSCDKTHTCPP |
| | | CPAPELLGGPSVFLFPPKPKDTLMISRTPEVTCVVVDVSHEDPEVKFN |
| | | WYVDGVEVHNAKTKPREEQYNSTYRVVSVLTVLHQDWLNGKEYKCKVS |
| | | NKALPAPIEKTISKAKGQPREPQVYTLPPSRDELTKNQVSLTCLVKGF |
| | | YPSDIAVEWESNGQPENNYKTTPPVLDSDGSFFLYSKLTVDKSRWQQG |
| | | NVFSCSVMHEALHNHYTQKSLSLSPGK |
| | | SEQ ID NO: 13 |
| | | 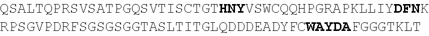 |
| | | VLRQPKAAPSVTLFPPSSEELQANKATLVCLISDFYPGAVTVAWKADSS |
| | | PVKAGVETTTPSKQSNNKYAASSYLSLTPEQWKSHRSYSCQVTHEGSTV |
| | | EKTVAPTECS |
| | | SEQ ID NO: 14 |
| Sequence 8 | N49P37 | 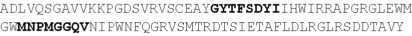 |
| | | YCVRDRSNGSGKRFESSNWFLDLWGRGTAVTISSPSTKGPSVFPLAP |
| | | SSKSTSGGTAALGCLVKDYFPEPVTVSWNSGALTSGVHTFPAVLQSS |
| | | GLYSLSSVVTVPSSSLGTQTYICNVNHKPSNTKVDKKVEPKSCDKTH |
| | | TCPPCPAPELLGGPSVFLFPPKPKDTLMISRTPEVTCVVVDVSHEDP |
| | | EVKFNWYVDGVEVHNAKTKPREEQYNSTYRVVSVLTVLHQDWLNGKE |
| | | YKCKVSNKALPAPIEKTISKAKGQPREPQVYTLPPSRDELTKNQVSL |
| | | TCLVKGFYPSDIAVEWESNGQPENNYKTTPPVLDSDGSFFLYSKLTV |
| | | DKSRWQQGNVFSCSVMHEALHNHYTQKSLSLSPGK |
| | | SEQ ID NO: 15 |
| | | 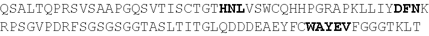 |
| | | VLRQPKAAPSVTLFPPSSEELQANKATLVCLISDFYPGAVTVAWKADSS |
| | | PVKAGVETTTPSKQSNNKYAASSYLSLTPEQWKSHRSYSCQVTHEGSTV |
| | | EKTVAPTECS |
| | | SEQ ID NO: 16 |
| Sequence 9 | N49P38 N49P38.1 | 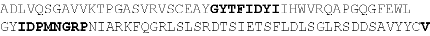 |
| | | RDKSNGSGKRFDSSNWFLDLWGRGTRVSISSASTKGPSVFPLAPSSKST |
| | | SGGTAALGCLVKDYFPEPVTVSWNSGALTSGVHTFPAVLQSSGLYSLSS |
| | | VVTVPSSSLGTQTYICNVNHKPSNTKVDKKVEPKSCDKTHTCPPCPAPE |
| | | LLGGPSVFLFPPKPKDTLMISRTPEVTCVVVDVSHEDPEVKFNWYVDGV |
| | | EVHNAKTKPREEQYNSTYRVVSVLTVLHQDWLNGKEYKCKVSNKALPAP |
| | | IEKTISKAKGQPREPQVYTLPPSRDELTKNQVSLTCLVKGFYPSDIAVE |
| | | WESNGQPENNYKTTPPVLDSDGSFFLYSKLTVDKSRWQQGNVFSCSVMH |
| | | EALHNHYTQKSLSLSPGK |
| | | SEQ ID NO: 17 |
| | | 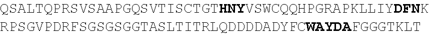 |
| | | VLRQPKAAPSVTLFPPSSEELQANKATLVCLISDFYPGAVTVAWKADSS |
| | | PVKAGVETTTPSKQSNNKYAASSYLSLTPEQWKSHRSYSCQVTHEGSTV |
| | | EKTVAPTECS |
| | | SEQ ID NO: 18 |
| Sequence 10 | N49P55 | 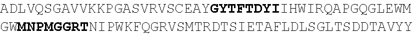 |
| | | CVRDKSNGSGKRFDSSNWFLDLWGRGTPVTISSPSTKGPSVFPLAPSS |
| | | KSTSGGTAALGCLVKDYFPEPVTVSWNSGALTSGVHTFPAVLQSSGLY |
| | | SLSSVVTVPSSSLGTQTYICNVNHKPSNTKVDKKVEPKSCDKTHTCPP |
| | | CPAPELLGGPSVFLFPPKPKDTLMISRTPEVTCVVVDVSHEDPEVKFN |
| | | WYVDGVEVHNAKTKPREEQYNSTYRVVSVLTVLHQDWLNGKEYKCKVS |
| | | NKALPAPIEKTISKAKGQPREPQVYTLPPSRDELTKNQVSLTCLVKGF |
| | | YPSDIAVEWESNGQPENNYKTTPPVLDSDGSFFLYSKLTVDKSRWQQG |
| | | NVFSCSVMHEALHNHYTQKSLSLSPGK |
| | | SEQ ID NO: 19 |
| | | 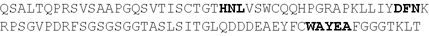 |
| | | VLRQPKAAPSVTLFPPSSEELQANKATLVCLISDFYPGAVTVAWKADSS |
| | | PVKAGVETTTPSKQSNNKYAASSYLSLTPEQWKSHRSYSCQVTHEGSTV |
| | | EKTVAPTECS |
| | | SEQ ID NO: 20 |
| Sequence 11 | N49P56 | 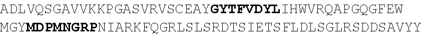 |
| | | CVRDKSGGSGKLFDSSNWFLDLWGRGTRVSISSASTKGPSVFPLAPSS |
| | | KSTSGGTAALGCLVKDYFPEPVTVSWNSGALTSGVHTFPAVLQSSGLY |
| | | SLSSVVTVPSSSLGTQTYICNVNHKPSNTKVDKKVEPKSCDKTHTCPP |
| | | CPAPELLGGPSVFLFPPKPKDTLMISRTPEVTCVVVDVSHEDPEVKFN |
| | | WYVDGVEVHNAKTKPREEQYNSTYRVVSVLTVLHQDWLNGKEYKCKVS |
| | | NKALPAPIEKTISKAKGQPREPQVYTLPPSRDELTKNQVSLTCLVKGF |
| | | YPSDIAVEWESNGQPENNYKTTPPVLDSDGSFFLYSKLTVDKSRWQQG |
| | | NVFSCSVMHEALHNHYTQKSLSLSPGK |
| | | SEQ ID NO: 21 |
| | | 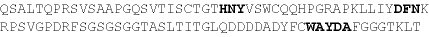 |
| | | VLRQPKAAPSVTLFPPSSEELQANKATLVCLISDFYPGAVTVAWKADSS |
| | | PVKAGVETTTPSKQSNNKYAASSYLSLTPEQWKSHRSYSCQVTHEGSTV |
| | | EKTVAPTECS |
| | | SEQ ID NO: 22 |
| Sequence 12 | N49P57 | 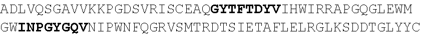 |
| | | VRDRSNGWGKRFESSNWFLDLWGRGTVITVHSPSTKGPSVFPLAPSS |
| | | KSTSGGTAALGCLVKDYFPEPVTVSWNSGALTSGVHTFPAVLQSSGLY |
| | | SLSSVVTVPSSSLGTQTYICNVNHKPSNTKVDKKVEPKSCDKTHTCPP |
| | | CPAPELLGGPSVFLFPPKPKDTLMISRTPEVTCVVVDVSHEDPEVKFN |
| | | WYVDGVEVHNAKTKPREEQYNSTYRVVSVLTVLHQDWLNGKEYKCKVS |
| | | NKALPAPIEKTISKAKGQPREPQVYTLPPSRDELTKNQVSLTCLVKGF |
| | | YPSDIAVEWESNGQPENNYKTTPPVLDSDGSFFLYSKLTVDKSRWQQG |
| | | NVFSCSVMHEALHNHYTQKSLSLSPGK |
| | | SEQ ID NO: 23 |
| | | 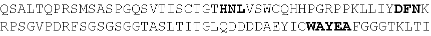 |
| | | LRQPKAAPSVTLFPPSSEELQANKATLVCLISDFYPGAVTVAWKADSSP |
| | | VKAGVETTTPSKQSNNKYAASSYLSLTPEQWKSHRSYSCQVTHEGSTVE |
| | | KTVAPTECS |
| | | SEQ ID NO: 24 |
| Sequence 13 | N49P58 | 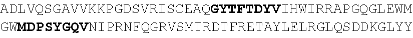 |
| | | CVRDRSHGSGRQFESSNWFLDLWGRGTVVNVQSPSTKGPSVFPLAPS |
| | | SKSTSGGTAALGCLVKDYFPEPVTVSWNSGALTSGVHTFPAVLQSSG |
| | | LYSLSSVVTVPSSSLGTQTYICNVNHKPSNTKVDKKVEPKSCDKTHT |
| | | CPPCPAPELLGGPSVFLFPPKPKDTLMISRTPEVTCVVVDVSHEDPE |
| | | VKFNWYVDGVEVHNAKTKPREEQYNSTYRVVSVLTVLHQDWLNGKEY |
| | | KCKVSNKALPAPIEKTISKAKGQPREPQVYTLPPSRDELTKNQVSLT |
| | | CLVKGFYPSDIAVEWESNGQPENNYKTTPPVLDSDGSFFLYSKLTVD |
| | | KSRWQQGNVFSCSVMHEALHNHYTQKSLSLSPGK |
| | | SEQ ID NO: 25 |
| | | 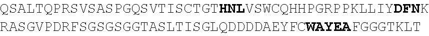 |
| | | VLRQPKAAPSVTLFPPSSEELQANKATLVCLISDFYPGAVTVAWKADSS |
| | | PVKAGVETTTPSKQSNNKYAASSYLSLTPEQWKSHRSYSCQVTHEGSTV |
| | | EKTVAPTECS |
| | | SEQ ID NO: 26 |
| Sequence 14 | N49P59 | 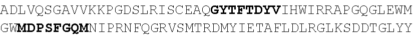 |
| | | CVRDRSHGSGRLFESSNWFLDLWGRGTVVTVQSPSTKGPSVFPLAPSS |
| | | KSTSGGTAALGCLVKDYFPEPVTVSWNSGALTSGVHTFPAVLQSSGLY |
| | | SLSSVVTVPSSSLGTQTYICNVNHKPSNTKVDKKVEPKSCDKTHTCPP |
| | | CPAPELLGGPSVFLFPPKPKDTLMISRTPEVTCVVVDVSHEDPEVKFN |
| | | WYVDGVEVHNAKTKPREEQYNSTYRVVSVLTVLHQDWLNGKEYKCKVS |
| | | NKALPAPIEKTISKAKGQPREPQVYTLPPSRDELTKNQVSLTCLVKGF |
| | | YPSDIAVEWESNGQPENNYKTTPPVLDSDGSFFLYSKLTVDKSRWQQG |
| | | NVFSCSVMHEALHNHYTQKSLSLSPGK |
| | | SEQ ID NO: 27 |
| | | 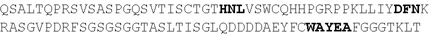 |
| | | VLRQPKAAPSVTLFPPSSEELQANKATLVCLISDFYPGAVTVAWKADSS |
| | | PVKAGVETTTPSKQSNNKYAASSYLSLTPEQWKSHRSYSCQVTHEGSTV |
| | | EKTVAPTECS |
| | | SEQ ID NO: 28 |
| Sequence 15 | N49P73 | 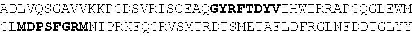 |
| | | CVRDRSHGSGRLFESSNWFLDLWGRGTVVTVQSPSTKGPSVFPLAPSS |
| | | KSTSGGTAALGCLVKDYFPEPVTVSWNSGALTSGVHTFPAVLQSSGLY |
| | | SLSSVVTVPSSSLGTQTYICNVNHKPSNTKVDKKVEPKSCDKTHTCPP |
| | | CPAPELLGGPSVFLFPPKPKDTLMISRTPEVTCVVVDVSHEDPEVKFN |
| | | WYVDGVEVHNAKTKPREEQYNSTYRVVSVLTVLHQDWLNGKEYKCKVS |
| | | NKALPAPIEKTISKAKGQPREPQVYTLPPSRDELTKNQVSLTCLVKGF |
| | | YPSDIAVEWESNGQPENNYKTTPPVLDSDGSFFLYSKLTVDKSRWQQG |
| | | NVFSCSVMHEALHNHYTQKSLSLSPGK |
| | | SEQ ID NO: 29 |
| | | 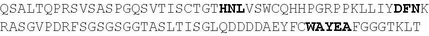 |
| | | VLRQPKAAPSVTLFPPSSEELQANKATLVCLISDFYPGAVTVAWKADSS |
| | | PVKAGVETTTPSKQSNNKYAASSYLSLTPEQWKSHRSYSCQVTHEGSTV |
| | | EKTVAPTECS |
| | | SEQ ID NO: 30 |
| Sequence 16 | N49P74 | 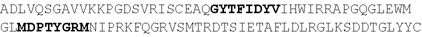 |
| | | VRDRSHGSGRLFESSNWFLDLWGRGTVVTVQSPSTKGPSVFPLAPSSK |
| | | STSGGTAALGCLVKDYFPEPVTVSWNSGALTSGVHTFPAVLQSSGLYS |
| | | LSSVVTVPSSSLGTQTYICNVNHKPSNTKVDKKVEPKSCDKTHTCPPC |
| | | PAPELLGGPSVFLFPPKPKDTLMISRTPEVTCVVVDVSHEDPEVKFNW |
| | | YVDGVEVHNAKTKPREEQYNSTYRVVSVLTVLHQDWLNGKEYKCKVSN |
| | | KALPAPIEKTISKAKGQPREPQVYTLPPSRDELTKNQVSLTCLVKGFY |
| | | PSDIAVEWESNGQPENNYKTTPPVLDSDGSFFLYSKLTVDKSRWQQGN |
| | | VFSCSVMHEALHNHYTQKSLSLSPGK |
| | | SEQ ID NO: 31 |
| | | 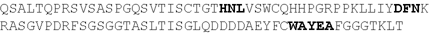 |
| | | VLRQPKAAPSVTLFPPSSEELQANKATLVCLISDFYPGAVTVAWKADSS |
| | | PVKAGVETTTPSKQSNNKYAASSYLSLTPEQWKSHRSYSCQVTHEGSTV |
| | | EKTVAPTECS |
| | | SEQ ID NO: 32 |
| Sequence 17 | N49P75 | 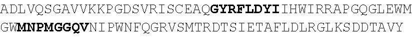 |
| | | YCVRDRSNGSGKRFESSNWFLDLWGRGTAVTIHSPSTKGPSVFPLAPS |
| | | SKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCP |
| | | PCPAPELLGGPSVFLFPPKPKDTLMISRTPEVTCVVVDVSHEDPEVKF |
| | | NWYVDGVEVHNAKTKPREEQYNSTYRVVSVLTVLHQDWLNGKEYKCKV |
| | | SNKALPAPIEKTISKAKGQPREPQVYTLPPSRDELTKNQVSLTCLVKG |
| | | FYPSDIAVEWESNGQPENNYKTTPPVLDSDGSFFLYSKLTVDKSRWQQ |
| | | GNVFSCSVMHEALHNHYTQKSLSLSPGK |
| | | SEQ ID NO: 33 |
| | | 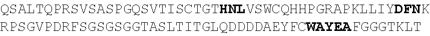 |
| | | VLRQPKAAPSVTLFPPSSEELQANKATLVCLISDFYPGAVTVAWKADSS |
| | | PVKAGVETTTPSKQSNNKYAASSYLSLTPEQWKSHRSYSCQVTHEGSTV |
| | | EKTVAPTECS |
| | | SEQ ID NO: 34 |
| Sequence 18 | N49P9 N49P9.1 | 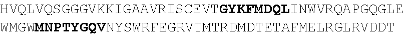 |
| | N49P9.2 | AVYYCARGPSGENYPFHYWGQGVRVVVSSPSTKGPSVFPLAPSSKSTS |
| | N49P917 | GGTAALGCLVKDYFPEPVTVSWNSGALTSGVHTFPAVLQSSGLYSLSS |
| | N49P9-FR | VVTVPSSSLGTQTYICNVNHKPSNTKVDKKVEPKSCDKTHTCPPCPAP |
| | | ELLGGPSVFLFPPKPKDTLMISRTPEVTCVVVDVSHEDPEVKFNWYVD |
| | | GVEVHNAKTKPREEQYNSTYRVVSVLTVLHQDWLNGKEYKCKVSNKAL |
| | | PAPIEKTISKAKGQPREPQVYTLPPSRDELTKNQVSLTCLVKGFYPSD |
| | | IAVEWESNGQPENNYKTTPPVLDSDGSFFLYSKLTVDKSRWQQGNVFS |
| | | CSVMHEALHNHYTQKSLSLSPGK |
| | | SEQ ID NO: 35 |
| | | 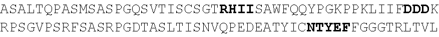 |
| | | SQPKAAPSVTLFPPSSEELQANKATLVCLVSDFYPGAVTVAWKADGSPV |
| | | KVGVETTKPSKQSNNKYAASSYLSLTPEQWKSHRSYSCRVTHEGSTVEK |
| | | TVAPAECS |
| | | SEQ ID NO: 36 |
| Sequence 19 | N49P22 | 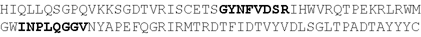 |
| | | ARGIDGKSYPFHFWGHGTRVTVFSASTKGPSVFPLAPSSKSTSGGTAA |
| | | LGCLVKDYFPEPVTVSWNSGALTSGVHTFPAVLQSSGLYSLSSVVTVP |
| | | SSSLGTQTYICNVNHKPSNTKVDKKVEPKSCDKTHTCPPCPAPELLGG |
| | | PSVFLFPPKPKDTLMISRTPEVTCVVVDVSHEDPEVKFNWYVDGVEVH |
| | | NAKTKPREEQYNSTYRVVSVLTVLHQDWLNGKEYKCKVSNKALPAPIE |
| | | KTISKAKGQPREPQVYTLPPSRDELTKNQVSLTCLVKGFYPSDIAVEW |
| | | ESNGQPENNYKTTPPVLDSDGSFFLYSKLTVDKSRWQQGNVFSCSVMH |
| | | EALHNHYTQKSLSLSPGK |
| | | SEQ ID NO: 37 |
| | | 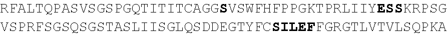 |
| | | APSVTLFPPSSEELQANKATLVCLVSDFYPGAVTVAWKADGSPVKVGVETT |
| | | KPSKQSNNKYAASSYLSLTPEQWKSHRSYSCRVTHEGSTVEKTVAPAECS |
| | | SEQ ID NO: 38 |
| Sequence 20 | N49P23 | 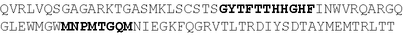 |
| | | GDTGTYYCARGDFGQNYPFHYWGQGSLVIVSSASTKGPSVFPLAPS |
| | | SKSTSGGTAALGCLVKDYFPEPVTVSWNSGALTSGVHTFPAVLQSS |
| | | GLYSLSSVVTVPSSSLGTQTYICNVNHKPSNTKVDKKVEPKSCDKT |
| | | HTCPPCPAPELLGGPSVFLFPPKPKDTLMISRTPEVTCVVVDVSHE |
| | | DPEVKFNWYVDGVEVHNAKTKPREEQYNSTYRVVSVLTVLHQDWLN |
| | | GKEYKCKVSNKALPAPIEKTISKAKGQPREPQVYTLPPSRDELTKN |
| | | QVSLTCLVKGFYPSDIAVEWESNGQPENNYKTTPPVLDSDGSFFLY |
| | | SKLTVDKSRWQQGNVFSCSVMHEALHNHYTQKSLSLSPGK |
| | | SEQ ID NO: 39 |
| | | 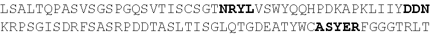 |
| | | VLSQPKAAPSVTLFPPSSEELQANKATLVCLVSDFYPGAVTVAWKADG |
| | | SPVKVGVETTKPSKQSNNKYAASSYLSLTPEQWKSHRSYSCRVTHEGS |
| | | TVEKTVAPAECS |
| | | SEQ ID NO: 40 |
| Sequence 21 | N49P9.3 N49P9.4 | 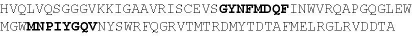 |
| | N49P9.6 | VYYCARGPSGENYPFHYWGQGVRVVVSSPSTKGPSVFPLAPSSKSTS |
| | N49P9.3-FR | GGTAALGCLVKDYFPEPVTVSWNSGALTSGVHTFPAVLQSSGLYSLS |
| | N49P9.6-FR | SVVTVPSSSLGTQTYICNVNHKPSNTKVDKKVEPKSCDKTHTCPPCP |
| | N49P9.6-FR-54W | APELLGGPSVFLFPPKPKDTLMISRTPEVTCVVVDVSHEDPEVKFNW |
| | N49P9.6-FR-54-F | YVDGVEVHNAKTKPREEQYNSTYRVVSVLTVLHQDWLNGKEYKCKVS |
| | N49P9.6-54W | NKALPAPIEKTISKAKGQPREPQVYTLPPSRDELTKNQVSLTCLVKG |
| | N49P9.6-54F | FYPSDIAVEWESNGQPENNYKTTPPVLDSDGSFFLYSKLTVDKSRWQ |
| | N49P9.6-FR3-06 | QGNVFSCSVMHEALHNHYTQKSLSLSPGK |
| | N49P9.6-FR1-D | SEQ ID NO: 41 |
| | N49P9.6-FR1-D-I | |
| | | 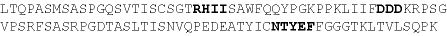 |
| | | AAPSVTLFPPSSEELQANKATLVCLVSDFYPGAVTVAWKADGSPVKVGV |
| | | ETTKPSKQSNNKYAASSYLSLTPEQWKSHRSYSCRVTHEGSTVEKTVAP |
| | | AECS |
| | | SEQ ID NO: 42 |
| Sequence 22 | N49P51 | 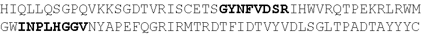 |
| | | ARGIDGKSYPFHFWGHGTRVTVFSASTKGPSVFPLAPSSKSTSGGTAA |
| | | LGCLVKDYFPEPVTVSWNSGALTSGVHTFPAVLQSSGLYSLSSVVTVP |
| | | SSSLGTQTYICNVNHKPSNTKVDKKVEPKSCDKTHTCPPCPAPELLGG |
| | | PSVFLFPPKPKDTLMISRTPEVTCVVVDVSHEDPEVKFNWYVDGVEVH |
| | | NAKTKPREEQYNSTYRVVSVLTVLHQDWLNGKEYKCKVSNKALPAPIE |
| | | KTISKAKGQPREPQVYTLPPSRDELTKNQVSLTCLVKGFYPSDIAVEW |
| | | ESNGQPENNYKTTPPVLDSDGSFFLYSKLTVDKSRWQQGNVFSCSVMH |
| | | EALHNHYTQKSLSLSPGK |
| | | SEQ ID NO: 43 |
| | | 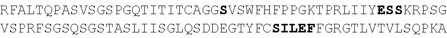 |
| | | APSVTLFPPSSEELQANKATLVCLVSDFYPGAVTVAWKADGSPVKVGV |
| | | ETTKPSKQSNNKYAASSYLSLTPEQWKSHRSYSCRVTHEGSTVEKTVA |
| | | PAECS |
| | | SEQ ID NO: 44 |
| Sequence 23 | N49P52 | 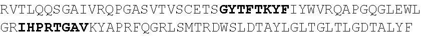 |
| | | CARGAFEADSYGSSYPFHHWGQGTLVTVSSASTKGPSVFPLAPSSKST |
| | | SGGTAALGCLVKDYFPEPVTVSWNSGALTSGVHTFPAVLQSSGLYSLS |
| | | SVVTVPSSSLGTQTYICNVNHKPSNTKVDKKVEPKSCDKTHTCPPCPA |
| | | PELLGGPSVFLFPPKPKDTLMISRTPEVTCVVVDVSHEDPEVKFNWYV |
| | | DGVEVHNAKTKPREEQYNSTYRVVSVLTVLHQDWLNGKEYKCKVSNKA |
| | | LPAPIEKTISKAKGQPREPQVYTLPPSRDELTKNQVSLTCLVKGFYPS |
| | | DIAVEWESNGQPENNYKTTPPVLDSDGSFFLYSKLTVDKSRWQQGNVF |
| | | SCSVMHEALHNHYTQKSLSLSPGK |
| | | SEQ ID NO: 45 |
| | | 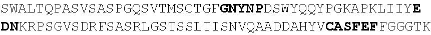 |
| | | LTVLSQPKAAPSVTLFPPSSEELQANKATLVCLVSDFYPGAVTVAWKA |
| | | DGSPVKVGVETTKPSKQSNNKYAASSYLSLTPEQWKSHRSYSCRVTHE |
| | | GSTVEKTVAPAECS |
| | | SEQ ID NO: 46 |
| Sequence 24 | N49P53 | 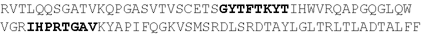 |
| | | CARGAFEADLSGPTYPFHHWGQGTLVIVSAASTKGPSVFPLAPSSKST |
| | | SGGTAALGCLVKDYFPEPVTVSWNSGALTSGVHTFPAVLQSSGLYSLS |
| | | SVVTVPSSSLGTQTYICNVNHKPSNTKVDKKVEPKSCDKTHTCPPCPA |
| | | PELLGGPSVFLFPPKPKDTLMISRTPEVTCVVVDVSHEDPEVKFNWYV |
| | | DGVEVHNAKTKPREEQYNSTYRVVSVLTVLHQDWLNGKEYKCKVSNKA |
| | | LPAPIEKTISKAKGQPREPQVYTLPPSRDELTKNQVSLTCLVKGFYPS |
| | | DIAVEWESNGQPENNYKTTPPVLDSDGSFFLYSKLTVDKSRWQQGNVF |
| | | SCSVMHEALHNHYTQKSLSLSPGK |
| | | SEQ ID NO: 47 |
| | | 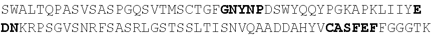 |
| | | LIVLSQPKAAPSVTLFPPSSEELQANKATLVCLVSDFYPGAVTVAWKAD |
| | | GSPVKVGVETTKPSKQSNNKYAASSYLSLTPEQWKSHRSYSCRVTHEGS |
| | | TVEKTVAPAECS |
| | | SEQ ID NO: 48 |
| Sequence 25 | N49P54 | 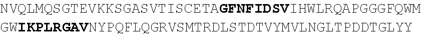 |
| | | CAKGAFRGGSPFGFWGQGTLLTVSPASTKGPSVFPLAPSSKSTSGGTA |
| | | ALGCLVKDYFPEPVTVSWNSGALTSGVHTFPAVLQSSGLYSLSSVVTV |
| | | PSSSLGTQTYICNVNHKPSNTKVDKKVEPKSCDKTHTCPPCPAPELLG |
| | | GPSVFLFPPKPKDTLMISRTPEVTCVVVDVSHEDPEVKFNWYVDGVEV |
| | | HNAKTKPREEQYNSTYRVVSVLTVLHQDWLNGKEYKCKVSNKALPAPI |
| | | EKTISKAKGQPREPQVYTLPPSRDELTKNQVSLTCLVKGFYPSDIAVE |
| | | WESNGQPENNYKTTPPVLDSDGSFFLYSKLTVDKSRWQQGNVFSCSVM |
| | | HEALHNHYTQKSLSLSPGK |
| | | SEQ ID NO: 49 |
| | | 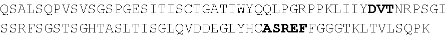 |
| | | AAPSVTLFPPSSEELQANKATLVCLVSDFYPGAVTVAWKADGSPVKVGV |
| | | ETTKPSKQSNNKYAASSYLSLTPEQWKSHRSYSCRVTHEGSTVEKTVAP |
| | | AECS |
| | | SEQ ID NO: 50 |
| Sequence 26 | N49P60 | 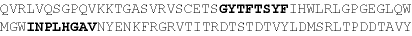 |
| | | FCTRGIVADGWPYGHWGQGTQVTVSPASTKGPSVFPLAPSSKSTSGGT |
| | | AALGCLVKDYFPEPVTVSWNSGALTSGVHTFPAVLQSSGLYSLSSVVT |
| | | VPSSSLGTQTYICNVNHKPSNTKVDKKVEPKSCDKTHTCPPCPAPELL |
| | | GGPSVFLFPPKPKDTLMISRTPEVTCVVVDVSHEDPEVKFNWYVDGVE |
| | | VHNAKTKPREEQYNSTYRVVSVLTVLHQDWLNGKEYKCKVSNKALPAP |
| | | IEKTISKAKGQPREPQVYTLPPSRDELTKNQVSLTCLVKGFYPSDIAV |
| | | EWESNGQPENNYKTTPPVLDSDGSFFLYSKLTVDKSRWQQGNVFSCSV |
| | | MHEALHNHYTQKSLSLSPGK |
| | | SEQ ID NO: 51 |
| | | 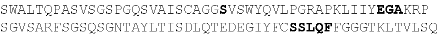 |
| | | PKAAPSVTLFPPSSEELQANKATLVCLVSDFYPGAVTVAWKADGSPVKV |
| | | GVETTKPSKQSNNKYAASSYLSLTPEQWKSHRSYSCRVTHEGSTVEKTV |
| | | APAECS |
| | | SEQ ID NO: 52 |
| Sequence 27 | N49P61 | 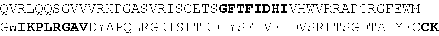 |
| | | AAAPEEAFPLQYWGQGTQLIVSSASTKGPSVFPLAPSSKSTSGGTAALG |
| | | CLVKDYFPEPVTVSWNSGALTSGVHTFPAVLQSSGLYSLSSVVTVPSSS |
| | | LGTQTYICNVNHKPSNTKVDKKVEPKSCDKTHTCPPCPAPELLGGPSVF |
| | | LFPPKPKDTLMISRTPEVTCVVVDVSHEDPEVKFNWYVDGVEVHNAKTK |
| | | PREEQYNSTYRVVSVLTVLHQDWLNGKEYKCKVSNKALPAPIEKTISKA |
| | | KGQPREPQVYTLPPSRDELTKNQVSLTCLVKGFYPSDIAVEWESNGQPE |
| | | NNYKTTPPVLDSDGSFFLYSKLTVDKSRWQQGNVFSCSVMHEALHNHYT |
| | | QKSLSLSPGK |
| | | SEQ ID NO: 53 |
| | | 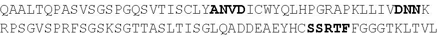 |
| | | SQPKAAPSVTLFPPSSEELQANKATLVCLVSDFYPGAVTVAWKADGSP |
| | | VKVGVETTKPSKQSNNKYAASSYLSLTPEQWKSHRSYSCRVTHEGSTV |
| | | EKTVAPAECS |
| | | SEQ ID NO: 54 |
| Sequence 28 | N49P62 | 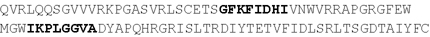 |
| | | CKAAAPDEAFPLEYWGQGTQLIVSPASTKGPSVFPLAPSSKSTSGGTAA |
| | | LGCLVKDYFPEPVTVSWNSGALTSGVHTFPAVLQSSGLYSLSSVVTVPS |
| | | SSLGTQTYICNVNHKPSNTKVDKKVEPKSCDKTHTCPPCPAPELLGGPS |
| | | VFLFPPKPKDTLMISRTPEVTCVVVDVSHEDPEVKFNWYVDGVEVHNAK |
| | | TKPREEQYNSTYRVVSVLTVLHQDWLNGKEYKCKVSNKALPAPIEKTIS |
| | | KAKGQPREPQVYTLPPSRDELTKNQVSLTCLVKGFYPSDIAVEWESNGQ |
| | | PENNYKTTPPVLDSDGSFFLYSKLTVDKSRWQQGNVFSCSVMHEALHNH |
| | | YTQKSLSLSPGK |
| | | SEQ ID NO: 55 |
| | | 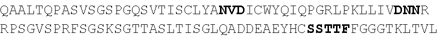 |
| | | SQPKAAPSVTLFPPSSEELQANKATLVCLVSDFYPGAVTVAWKADGSP |
| | | VKVGVETTKPSKQSNNKYAASSYLSLTPEQWKSHRSYSCRVTHEGSTV |
| | | EKTVAPAECS |
| | | SEQ ID NO: 56 |
| Sequence 29 | N49P63 | 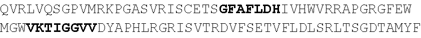 |
| | | CSKAAAPDEAFPLEFWGQGTQVIVSSASTKGPSVFPLAPSSKSTSGGTA |
| | | ALGCLVKDYFPEPVTVSWNSGALTSGVHTFPAVLQSSGLYSLSSVVTVP |
| | | SSSLGTQTYICNVNHKPSNTKVDKKVEPKSCDKTHTCPPCPAPELLGGP |
| | | SVFLFPPKPKDTLMISRTPEVTCVVVDVSHEDPEVKFNWYVDGVEVHNA |
| | | KTKPREEQYNSTYRVVSVLTVLHQDWLNGKEYKCKVSNKALPAPIEKTI |
| | | SKAKGQPREPQVYTLPPSRDELTKNQVSLTCLVKGFYPSDIAVEWESNG |
| | | QPENNYKTTPPVLDSDGSFFLYSKLTVDKSRWQQGNVFSCSVMHEALHN |
| | | HYTQKSLSLSPGK |
| | | SEQ ID NO: 57 |
| | | 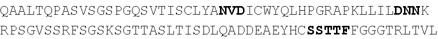 |
| | | SQPKAAPSVTLFPPSSEELQANKATLVCLVSDFYPGAVTVAWKADGSP |
| | | VKVGVETTKPSKQSNNKYAASSYLSLTPEQWKSHRSYSCRVTHEGSTV |
| | | EKTVAPAECS |
| | | SEQ ID NO: 58 |
| Sequence 30 | N49P64 | 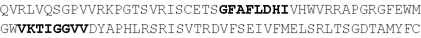 |
| | | SKAAAPDEAFPLEFWGQGTQVIVSSASTKGPSVFPLAPSSKSTSGGTAA |
| | | LGCLVKDYFPEPVTVSWNSGALTSGVHTFPAVLQSSGLYSLSSVVTVPS |
| | | SSLGTQTYICNVNHKPSNTKVDKKVEPKSCDKTHTCPPCPAPELLGGPS |
| | | VFLFPPKPKDTLMISRTPEVTCVVVDVSHEDPEVKFNWYVDGVEVHNAK |
| | | TKPREEQYNSTYRVVSVLTVLHQDWLNGKEYKCKVSNKALPAPIEKTIS |
| | | KAKGQPREPQVYTLPPSRDELTKNQVSLTCLVKGFYPSDIAVEWESNGQ |
| | | PENNYKTTPPVLDSDGSFFLYSKLTVDKSRWQQGNVFSCSVMHEALHNH |
| | | YTQKSLSLSPGK |
| | | SEQ ID NO: 59 |
| | | 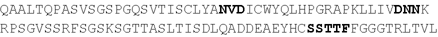 |
| | | SQPKAAPSVTLFPPSSEELQANKATLVCLVSDFYPGAVTVAWKADGSP |
| | | VKVGVETTKPSKQSNNKYAASSYLSLTPEQWKSHRSYSCRVTHEGSTV |
| | | EKTVAPAECS |
| | | SEQ ID NO: 60 |
| Sequence 31 | N49P65 | 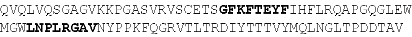 |
| | | YYCARAVFNEAFPFDYWGQGSLVTVSSASTKGPSVFPLAPSSKSTSGG |
| | | TAALGCLVKDYFPEPVTVSWNSGALTSGVHTFPAVLQSSGLYSLSSVVT |
| | | VPSSSLGTQTYICNVNHKPSNTKVDKKVEPKSCDKTHTCPPCPAPELLG |
| | | GPSVFLFPPKPKDTLMISRTPEVTCVVVDVSHEDPEVKFNWYVDGVEVH |
| | | NAKTKPREEQYNSTYRVVSVLTVLHQDWLNGKEYKCKVSNKALPAPIEK |
| | | TISKAKGQPREPQVYTLPPSRDELTKNQVSLTCLVKGFYPSDIAVEWES |
| | | NGQPENNYKTTPPVLDSDGSFFLYSKLTVDKSRWQQGNVFSCSVMHEAL |
| | | HNHYTQKSLSLSPGK |
| | | SEQ ID NO: 61 |
| | | 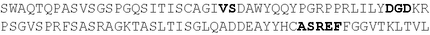 |
| | | SQPKAAPSVTLFPPSSEELQANKATLVCLVSDFYPGAVTVAWKADGSP |
| | | VKVGVETTKPSKQSNNKYAASSYLSLTPEQWKSHRSYSCRVTHEGSTV |
| | | EKTVAPAECS |
| | | SEQ ID NO: 62 |
| Sequence 32 | N49P66 | 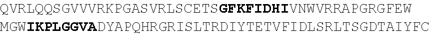 |
| | | CKAAAPDEAFPLEYWGQGTQLIVSPASTKGPSVFPLAPSSKSTSGGTAA |
| | | LGCLVKDYFPEPVTVSWNSGALTSGVHTFPAVLQSSGLYSLSSVVTVPS |
| | | SSLGTQTYICNVNHKPSNTKVDKKVEPKSCDKTHTCPPCPAPELLGGPS |
| | | VFLFPPKPKDTLMISRTPEVTCVVVDVSHEDPEVKFNWYVDGVEVHNAK |
| | | TKPREEQYNSTYRVVSVLTVLHQDWLNGKEYKCKVSNKALPAPIEKTIS |
| | | KAKGQPREPQVYTLPPSRDELTKNQVSLTCLVKGFYPSDIAVEWESNGQ |
| | | PENNYKTTPPVLDSDGSFFLYSKLTVDKSRWQQGNVFSCSVMHEALHNH |
| | | YTQKSLSLSPGK |
| | | SEQ ID NO: 63 |
| | | 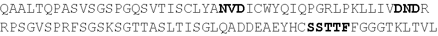 |
| | | SQPKAAPSVTLFPPSSEELQANKATLVCLVSDFYPGAVTVAWKADGSP |
| | | VKVGVETTKPSKQSNNKYAASSYLSLTPEQWKSHRSYSCRVTHEGSTV |
| | | EKTVAPAECS |
| | | SEQ ID NO: 64 |
| Sequence 33 | N49P67 | 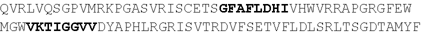 |
| | | CSKAAAPDEAFPLEFWGQGTQVIVSSASTKGPSVFPLAPSSKSTSGGTA |
| | | ALGCLVKDYFPEPVTVSWNSGALTSGVHTFPAVLQSSGLYSLSSVVTVP |
| | | SSSLGTQTYICNVNHKPSNTKVDKKVEPKSCDKTHTCPPCPAPELLGGP |
| | | SVFLFPPKPKDTLMISRTPEVTCVVVDVSHEDPEVKFNWYVDGVEVHNA |
| | | KTKPREEQYNSTYRVVSVLTVLHQDWLNGKEYKCKVSNKALPAPIEKTI |
| | | SKAKGQPREPQVYTLPPSRDELTKNQVSLTCLVKGFYPSDIAVEWESNG |
| | | QPENNYKTTPPVLDSDGSFFLYSKLTVDKSRWQQGNVFSCSVMHEALHN |
| | | HYTQKSLSLSPGK |
| | | SEQ ID NO: 65 |
| | | 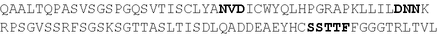 |
| | | SQPKAAPSVTLFPPSSEELQANKATLVCLVSDFYPGAVTVAWKADGSP |
| | | VKVGVETTKPSKQSNNKYAASSYLSLTPEQWKSHRSYSCRVTHEGSTV |
| | | EKTVAPAECS |
| | | SEQ ID NO: 66 |
| Sequence 34 | N49P68 | 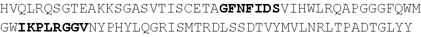 |
| | | CAKGAFGGSSPFGFWGQGTLLTVSPASTKGPSVFPLAPSSKSTSGGTA |
| | | ALGCLVKDYFPEPVTVSWNSGALTSGVHTFPAVLQSSGLYSLSSVVTVP |
| | | SSSLGTQTYICNVNHKPSNTKVDKKVEPKSCDKTHTCPPCPAPELLGGP |
| | | SVFLFPPKPKDTLMISRTPEVTCVVVDVSHEDPEVKFNWYVDGVEVHNA |
| | | KTKPREEQYNSTYRVVSVLTVLHQDWLNGKEYKCKVSNKALPAPIEKTI |
| | | SKAKGQPREPQVYTLPPSRDELTKNQVSLTCLVKGFYPSDIAVEWESNG |
| | | QPENNYKTTPPVLDSDGSFFLYSKLTVDKSRWQQGNVFSCSVMHEALHN |
| | | HYTQKSLSLSPGK |
| | | SEQ ID NO: 67 |
| | | 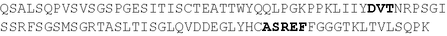 |
| | | AAPSVTLFPPSSEELQANKATLVCLVSDFYPGAVTVAWKADGSPVKVG |
| | | VETTKPSKQSNNKYAASSYLSLTPEQWKSHRSYSCRVTHEGSTVEKTV |
| | | APAECS |
| | | SEQ ID NO: 68 |
| Sequence 35 | N49P69 | 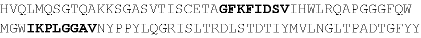 |
| | | CAKGAFGGGSPFGFWGQGTLLTVSPASTKGPSVFPLAPSSKSTSGGTA |
| | | ALGCLVKDYFPEPVTVSWNSGALTSGVHTFPAVLQSSGLYSLSSVVTVP |
| | | SSSLGTQTYICNVNHKPSNTKVDKKVEPKSCDKTHTCPPCPAPELLGGP |
| | | SVFLFPPKPKDTLMISRTPEVTCVVVDVSHEDPEVKFNWYVDGVEVHNA |
| | | KTKPREEQYNSTYRVVSVLTVLHQDWLNGKEYKCKVSNKALPAPIEKTI |
| | | SKAKGQPREPQVYTLPPSRDELTKNQVSLTCLVKGFYPSDIAVEWESNG |
| | | QPENNYKTTPPVLDSDGSFFLYSKLTVDKSRWQQGNVFSCSVMHEALHN |
| | | HYTQKSLSLSPGK |
| | | SEQ ID NO: 69 |
| | | 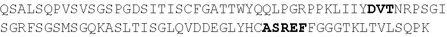 |
| | | AAPSVTLFPPSSEELQANKATLVCLVSDFYPGAVTVAWKADGSPVKVG |
| | | VETTKPSKQSNNKYAASSYLSLTPEQWKSHRSYSCRVTHEGSTVEKTV |
| | | APAECS |
| | | SEQ ID NO: 70 |
| Sequence 36 | N49P70 | 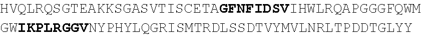 |
| | | CAKGAFGGSSPFGFWGQGTLLTVSPASTKGPSVFPLAPSSKSTSGGTA |
| | | ALGCLVKDYFPEPVTVSWNSGALTSGVHTFPAVLQSSGLYSLSSVVTVP |
| | | SSSLGTQTYICNVNHKPSNTKVDKKVEPKSCDKTHTCPPCPAPELLGGP |
| | | SVFLFPPKPKDTLMISRTPEVTCVVVDVSHEDPEVKFNWYVDGVEVHNA |
| | | KTKPREEQYNSTYRVVSVLTVLHQDWLNGKEYKCKVSNKALPAPIEKTI |
| | | SKAKGQPREPQVYTLPPSRDELTKNQVSLTCLVKGFYPSDIAVEWESNG |
| | | QPENNYKTTPPVLDSDGSFFLYSKLTVDKSRWQQGNVFSCSVMHEALHN |
| | | HYTQKSLSLSPGK |
| | | SEQ ID NO: 71 |
| | | 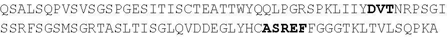 |
| | | APSVTLFPPSSEELQANKATLVCLVSDFYPGAVTVAWKADGSPVKVGV |
| | | ETTKPSKQSNNKYAASSYLSLTPEQWKSHRSYSCRVTHEGSTVEKTVA |
| | | PAECS |
| | | SEQ ID NO: 72 |
| Sequence 37 | N49P71 | 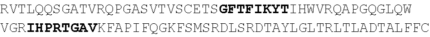 |
| | | ARGAFEADLYGPTYPFHHWGQGTQVTVSAASTKGPSVFPLAPSSKSTS |
| | | GGTAALGCLVKDYFPEPVTVSWNSGALTSGVHTFPAVLQSSGLYSLSS |
| | | VVTVPSSSLGTQTYICNVNHKPSNTKVDKKVEPKSCDKTHTCPPCPAP |
| | | ELLGGPSVFLFPPKPKDTLMISRTPEVTCVVVDVSHEDPEVKFNWYVD |
| | | GVEVHNAKTKPREEQYNSTYRVVSVLTVLHQDWLNGKEYKCKVSNKAL |
| | | PAPIEKTISKAKGQPREPQVYTLPPSRDELTKNQVSLTCLVKGFYPSD |
| | | VIAEWESNGQPENNYKTTPPVLDSDGSFFLYSKLTVDKSRWQQGNVFS |
| | | CSVMHEALHNHYTQKSLSLSPGK |
| | | SEQ ID NO: 73 |
| | | 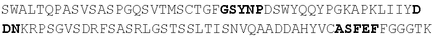 |
| | | LTVLSQPKAAPSVTLFPPSSEELQANKATLVCLVSDFYPGAVTVAWKA |
| | | DGSPVKVGVETTKPSKQSNNKYAASSYLSLTPEQWKSHRSYSCRVTHE |
| | | GSTVEKTVAPAECS |
| | | SEQ ID NO: 74 |
| Sequence 38 | N49P72 | 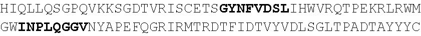 |
| | | ARGIDGNSYPFHFWGHGTRVTVFSASTKGPSVFPLAPSSKSTSGGTAAL |
| | | GCLVKDYFPEPVTVSWNSGALTSGVHTFPAVLQSSGLYSLSSVVTVPSS |
| | | SLGTQTYICNVNHKPSNTKVDKKVEPKSCDKTHTCPPCPAPELLGGPSV |
| | | FLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPAPIEKTISK |
| | | AKGQPREPQVYTLPPSRDELTKNQVSLTCLVKGFYPSDIAVEWESNGQP |
| | | ENNYKTTPPVLDSDGSFFLYSKLTVDKSRWQQGNVFSCSVMHEALHNHY |
| | | TQKSLSLSPGK |
| | | SEQ ID NO: 75 |
| | | 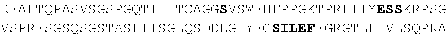 |
| | | APSVTLFPPSSEELQANKATLVCLVSDFYPGAVTVAWKADGSPVKVGV |
| | | ETTKPSKQSNNKYAASSYLSLTPEQWKSHRSYSCRVTHEGSTVEKTVA |
| | | PAECS |
| | | SEQ ID NO: 76 |

TABLE 2
Nucleotide sequences of natural antibodies.
Heavy chain is shown first (above dotted line),
followed by the light chain (below dotted line).

| Natural | Related | |
|---|---|---|
| antibody | variants | Nucleotide sequence |
| Sequence 1 | N49P6, | gcgggactgatgcagtctggggctgtgatgaagaattcgggggcctcagtgagggtctcttgtcaggctgat |
| | N49P6.2 | ggatacgacttcattgactatgtcattcactggtttcgacaaagacgtggagaaggtcttgagtggctggga |
| | N49P6-LS1 | tggatgaatccctcgggaggcggcacaaactatccgcgaccatttcagggcaaagtcaccatgaccagggac |
| | N49P6-LS2 | acgtccaccgagacagcctatttagatgtcagaggacttacatatgacgacacggccgtctattattgtgtg |
| | N49P6A | agagacagggccaacggttcgggaagaagacgttttgagtcggtgaattggttcctggatctgtggggccgc |
| | N49P6S | ggcacccaaataacagtcgtctcgccctccaccaagggcccatcggtcttccccctggcaccctcctccaag |
| | N49P6F | agcacctctgggggcacagcggccctgggctgcctggtcaaggactacttccccgaaccggtgacggtgtcg |
| | N49P6Y | tggaactcaggcgccctgaccagcggcgtgcacaccttcccggctgtcctacagtcctcaggactctactcc |
| | N49P6.54TY | ctcagcagcgtggtgaccgtgccctccagcagcttgggcacccagacctacatctgcaacgtgaatcacaag |
| | | cccagcaacaccaaggtggacaagaaagttgagcccaaatcttgtgacaaaactcacacatgcccaccgtgc |
| | | ccagcacctgaactcctggggggaccgtcagtcttcctcttccccccaaaacccaaggacaccctcatgatc |
| | | tcccggacccctgaggtcacatgcgtggtggtggacgtgagccacgaagaccctgaggtcaagttcaactgg |
| | | tacgtggacggcgtggaggtgcataatgccaagacaaagccgcgggaggagcagtacaacagcacgtaccgg |
| | | gtggtcagcgtcctcaccgtcctgcaccaggactggctgaatggcaaggagtacaagtgcaaggtctccaac |
| | | aaagccctcccagcccccatcgagaaaaccatctccaaagccaaagggcagccccgagaaccacaggtgtac |
| | | accctgcccccatcccgggatgagctgaccaagaaccaggtcagcctgacctgcctggtcaaaggcttctat |
| | | cccagcgacatcgccgtggagtgggagagcaatgggcagccggagaacaactacaagaccacgcctcccgtg |
| | | ctggactccgacggctccttcttcctctacagcaagctcaccgtggacaagagcaggtggcagcaggggaac |
| | | gtcttctcatgctccgtgatgcatgaggctctgcacaaccactacacgcagaagagcctctccctgtctccg |
| | | ggtaaa |
| | | SEQ ID NO: 77 |
| | | ------------------------------------------------------------------------ |
| | | -------------------------------------- |
| | | cagtctgccctaactcagcctcgctcagtgtccgcatctcctggtcagtcagtcaccatctcctgcactgga |
| | | acacacaattatgtgtcctggtgtcaacagaaaccgggccaagcccccaaattattaatttacgatttcaat |
| | | aaacggccctcaggggtctctgatcgcttctctggctccacgtctggcaacacggcctccctgaccatctct |
| | | ggactccaggctgacgatgagggtcattatttttgttgggcgtttgaaaatatcggcggagggaccaagctg |
| | | accgtcctgcgtcagcccaaggctgccccctcggtcactctgttcccgccctcctctgaggagcttcaagcc |
| | | aacaaggccacactggtgtgtctcataagtgacttctacccgggagccgtgacagtggcttggaaagcagat |
| | | agcagccccgtcaaggcgggagtggagaccaccacaccctccaaacaaagcaacaacaagtacgcggccagc |
| | | agctatctgagcctgacgcctgagcagtggaagtcccacagaagctacagctgccaggtcacgcatgaaggg |
| | | agcaccgtggagaagacagtggcccctacagaatgttca |
| | | SEQ ID NO: 78 |
| Sequence 2 | N49P7, | gcggacttggtgcagtctggggctgtggtgaagaagcctggggactcagtgaggatctcctgtgaggctcaa |
| | N49P7.1 | ggatatagatttcctgactacatcattcactggattcgacgggcccctggacaaggccctgaatggatggga |
| | N49P7A | tggatgaatccaatgggcggacaagtaaatattccatggaaatttcagggtagggtctccatgacccgggac |
| | N49P7S | acgtccatcgaaacagcatttctggacttaagaggactaaagtctgacgacacggccgtctattattgcgtg |
| | N49P7F | agagatcgcagtaatggatcgggaaagcgattcgagtcctccaattggttcctcgatctgtggggccgtggg |
| | N49P7Y | actgcggtcacaattcaatcatcctccaccaagggcccatcggtcttccccctggcaccctcctccaagagc |
| | N49P7.54TY | acctctgggggcacagcggccctgggctgcctggtcaaggactacttccccgaaccggtgacggtgtcgtgg |
| | N49P7-LS1 | aactcaggcgccctgaccagcggcgtgcacaccttcccggctgtcctacagtcctcaggactctactccctc |
| | N49P7-LS2 | agcagcgtggtgaccgtgccctccagcagcttgggcacccagacctacatctgcaacgtgaatcacaagccc |
| | N49P7/6L | agcaacaccaaggtggacaagaaagttgagcccaaatcttgtgacaaaactcacacatgcccaccgtgccca |
| | N49P7/11L | gcacctgaactcctggggggaccgtcagtcttcctcttccccccaaaacccaaggacaccctcatgatctcc |
| | R49P7 | cggacccctgaggtcacatgcgtggtggtggacgtgagccacgaagaccctgaggtcaagttcaactggtac |
| | N49P7-FR | gtggacggcgtggaggtgcataatgccaagacaaagccgcgggaggagcagtacaacagcacgtaccgggtg |
| | | gtcagcgtcctcaccgtcctgcaccaggactggctgaatggcaaggagtacaagtgcaaggtctccaacaaa |
| | | gccctcccagcccccatcgagaaaaccatctccaaagccaaagggcagccccgagaaccacaggtgtacacc |
| | | ctgcccccatcccgggatgagctgaccaagaaccaggtcagcctgacctgcctggtcaaaggcttctatccc |
| | | agcgacatcgccgtggagtgggagagcaatgggcagccggagaacaactacaagaccacgcctcccgtgctg |
| | | gactccgacggctccttcttcctctacagcaagctcaccgtggacaagagcaggtggcagcaggggaacgtc |
| | | ttctcatgctccgtgatgcatgaggctctgcacaaccactacacgcagaagagcctctccctgtctccgggt |
| | | aaa |
| | | SEQ ID NO: 79 |
| | | ------------------------------------------------------------------------ |
| | | -------------------------------------- |
| | | gccctgactcagcctcgctcagtgtccgcatctcctgggcagtccgtcaccatttcctgcactggaacccac |
| | | aatttggtctcttggtgtcaacatcagccaggcagagcccccaaattattaatttatgacttcaataagagg |
| | | ccctcaggggtccctgatcgcttctctggctccgggtctggcggcacggcctccctgaccatcactggactc |
| | | caggatgacgatgacgccgaatatttttgttgggcgtatgaagcttttggcggagggaccaagttgaccgtt |
| | | cttcgtcagcccaaggctgccccctcggtcactctgttcccgccctcctctgaggagcttcaagccaacaag |
| | | gccacactggtgtgtctcataagtgacttctacccgggagccgtgacagtggcttggaaagcagatagcagc |
| | | cccgtcaaggcgggagtggagaccaccacaccctccaaacaaagcaacaacaagtacgcggccagcagctat |
| | | ctgagcctgacgcctgagcagtggaagtcccacagaagctacagctgccaggtcacgcatgaagggagcacc |
| | | gtggagaagacagtggcccctacagaatgttca |
| | | SEQ ID NO: 80 |
| Sequence 3 | N49P7.2 | gcggacttggtgcagtctggggctgtggtgaagaagcctggggactcagtgaggatctcctgtgaggctcaa |
| | | ggatacaaatttcctgactacatcattcactggattcgacgggcccctggacaaggccttgagtggatgggg |
| | | tggattaatccaatgggcggacaagtaaacattccatggcagtttcagggcagggtctccatgacccgggac |
| | | acgtccatcgaaacagcatttctggacttaagaggactaaagtctgacgacacggccctctattattgcgtg |
| | | agagatcgaagtaatggatcgggaaggcgattcgagtcctccaattggttcctcgatctgtggggccgcggc |
| | | actgcggtcactgttcattcaccctccaccaagggcccatcggtcttccccctggcaccctcctccaagagc |
| | | acctctgggggcacagcggccctgggctgcctggtcaaggactacttccccgaaccggtgacggtgtcgtgg |
| | | aactcaggcgccctgaccagcggcgtgcacaccttcccggctgtcctacagtcctcaggactctactccctc |
| | | agcagcgtggtgaccgtgccctccagcagcttgggcacccagacctacatctgcaacgtgaatcacaagccc |
| | | agcaacaccaaggtggacaagaaagttgagcccaaatcttgtgacaaaactcacacatgcccaccgtgccca |
| | | gcacctgaactcctggggggaccgtcagtcttcctcttccccccaaaacccaaggacaccctcatgatctcc |
| | | cggacccctgaggtcacatgcgtggtggtggacgtgagccacgaagaccctgaggtcaagttcaactggtac |
| | | gtggacggcgtggaggtgcataatgccaagacaaagccgcgggaggagcagtacaacagcacgtaccgggtg |
| | | gtcagcgtcctcaccgtcctgcaccaggactggctgaatggcaaggagtacaagtgcaaggtctccaacaaa |
| | | gccctcccagcccccatcgagaaaaccatctccaaagccaaagggcagccccgagaaccacaggtgtacacc |
| | | ctgcccccatcccgggatgagctgaccaagaaccaggtcagcctgacctgcctggtcaaaggcttctatccc |
| | | agcgacatcgccgtggagtgggagagcaatgggcagccggagaacaactacaagaccacgcctcccgtgctg |
| | | gactccgacggctccttcttcctctacagcaagctcaccgtggacaagagcaggtggcagcaggggaacgtc |
| | | ttctcatgctccgtgatgcatgaggctctgcacaaccactacacgcagaagagcctctccctgtctccgggt |
| | | aaa |
| | | SEQ ID NO: 81 |
| | | ------------------------------------------------------------------------ |
| | | -------------------------------------- |
| | | cagtctgccctgactcagcctcgctcagtgtccgcatctcctgggcagtccgtcaccatttcctgcactgga |
| | | acccacaatttggtctcttggtgtcaacatcacccaggcagagcccccaaattattaatttatgacttcaat |
| | | aagaggccctcaggggtccctgatcgcttctctggctccgggtctggcggcacggcctccctgaccatcagt |
| | | ggactccaggatgacgatgacgccgaatatttttgttgggcgtatgaagcttttggcggagggaccaagttg |
| | | accgtacttcgtcagcccaaggctgccccctcggtcactctgttcccgccctcctctgaggagcttcaagcc |
| | | aacaaggccacactggtgtgtctcataagtgacttctacccgggagccgtgacagtggcttggaaagcagat |
| | | agcagccccgtcaaggcgggagtggagaccaccacaccctccaaacaaagcaacaacaagtacgcggccagc |
| | | agctatctgagcctgacgcctgagcagtggaagtcccacagaagctacagctgccaggtcacgcatgaaggg |
| | | agcaccgtggagaagacagtggcccctacagaatgttca |
| | | SEQ ID NO: 82 |
| Sequence 4 | N49P11 | tcggcggaattggtgcaatctggggctgtggtgaagaagcctgggacctccgtgaaggtctcttgtcaggct |
| | | tatggatacacttttaccgactaccttattcattggcttcgacaggcccctggacaaggacttgaatggatg |
| | | ggatggatgaatcctgtttatggacaagtaaattatgcccaaaactttcagggcagggtctccatgaccagg |
| | | gacatttacagggaaacagcatttctagaggtgcgcgacctgaagactgacgacacaggcacttattattgt |
| | | gtgagagacacaggcgacggttcgcggagacactttgactccatcaattggtttctcgatctttggggccgc |
| | | gggacatggataagggtcgccccagcctccaccaagggcccatcggtcttccccctggcaccctcctccaag |
| | | agcacctctgggggcacagcggccctgggctgcctggtcaaggactacttccccgaaccggtgacggtgtcg |
| | | tggaactcaggcgccctgaccagcggcgtgcacaccttcccggctgtcctacagtcctcaggactctactcc |
| | | ctcagcagcgtggtgaccgtgccctccagcagcttgggcacccagacctacatctgcaacgtgaatcacaag |
| | | cccagcaacaccaaggtggacaagaaagttgagcccaaatcttgtgacaaaactcacacatgcccaccgtgc |
| | | ccagcacctgaactcctggggggaccgtcagtcttcctcttccccccaaaacccaaggacaccctcatgatc |
| | | tcccggacccctgaggtcacatgcgtggtggtggacgtgagccacgaagaccctgaggtcaagttcaactgg |
| | | tacgtggacggcgtggaggtgcataatgccaagacaaagccgcgggaggagcagtacaacagcacgtaccgg |
| | | gtggtcagcgtcctcaccgtcctgcaccaggactggctgaatggcaaggagtacaagtgcaaggtctccaac |
| | | aaagccctcccagcccccatcgagaaaaccatctccaaagccaaagggcagccccgagaaccacaggtgtac |
| | | accctgcccccatcccgggatgagctgaccaagaaccaggtcagcctgacctgcctggtcaaaggcttctat |
| | | cccagcgacatcgccgtggagtgggagagcaatgggcagccggagaacaactacaagaccacgcctcccgtg |
| | | ctggactccgacggctccttcttcctctacagcaagctcaccgtggacaagagcaggtggcagcaggggaac |
| | | gtcttctcatgctccgtgatgcatgaggctctgcacaaccactacacgcagaagagcctctccctgtctccg |
| | | ggtaaa |
| | | SEQ ID NO: 83 |
| | | ------------------------------------------------------------------------ |
| | | -------------------------------------- |
| | | cagtgtgtcttgactcagcctcgctcagtgtccggatctcctggacaatcagtcaccatctcctgcactgga |
| | | actcacaattatgtctcctggtgtcaacaccacccaggcaacgcccccaaattattactttatgatttcgac |
| | | aagcggccctcaggaatctctgatcgcttctctggctctaggtctggcaacacggcctccctgaccatctct |
| | | ggcctccagcctgaggatgaggccgattacttttgttgggcctttgaagcctttggcggagggaccaaggtg |
| | | ctcgtccttcgtcagcccaaggctgccccctcggtcactctgttcccgccctcctctgaggagcttcaagcc |
| | | aacaaggccacactggtgtgtctcataagtgacttctacccgggagccgtgacagtggcttggaaagcagat |
| | | agcagccccgtcaaggcgggagtggagaccaccacaccctccaaacaaagcaacaacaagtacgcggccagc |
| | | agctatctgagcctgacgcctgagcagtggaagtcccacagaagctacagctgccaggtcacgcatgaaggg |
| | | agcaccgtggagaagacagtggcccctacagaatgttca |
| | | SEQ ID NO: 84 |
| Sequence 5 | N49P18 | gcggacttggtgcagtctggggctgtgatgaagaagcctggggactcagtgagaatctcctgtgaggctcga |
| | N49P18.2 | ggatacacattcactgactacgtcattcactggattcgacgggcccctggacaaggccttgaatggatgggg |
| | | tggattgatccaccttatggacaagtaaatattccatggaattttcagggcagggtctccatgacccgggac |
| | | acgtccatcgaaacagcatttctggacttaagaggtctaaagtctgacgacacgggcctctattattgcgtg |
| | | agagatcgaagtaatggatggggaaagcgattcgagtcctccaattggttcctcgatctgtggggccgcggc |
| | | actgtggtcactgttcactcaccctccaccaagggcccatcggtcttccccctggcaccctcctccaagagc |
| | | acctctgggggcacagcggccctgggctgcctggtcaaggactacttccccgaaccggtgacggtgtcgtgg |
| | | aactcaggcgccctgaccagcggcgtgcacaccttcccggctgtcctacagtcctcaggactctactccctc |
| | | agcagcgtggtgaccgtgccctccagcagcttgggcacccagacctacatctgcaacgtgaatcacaagccc |
| | | agcaacaccaaggtggacaagaaagttgagcccaaatcttgtgacaaaactcacacatgcccaccgtgccca |
| | | gcacctgaactcctggggggaccgtcagtcttcctcttccccccaaaacccaaggacaccctcatgatctcc |
| | | cggacccctgaggtcacatgcgtggtggtggacgtgagccacgaagaccctgaggtcaagttcaactggtac |
| | | gtggacggcgtggaggtgcataatgccaagacaaagccgcgggaggagcagtacaacagcacgtaccgggtg |
| | | gtcagcgtcctcaccgtcctgcaccaggactggctgaatggcaaggagtacaagtgcaaggtctccaacaaa |
| | | gccctcccagcccccatcgagaaaaccatctccaaagccaaagggcagccccgagaaccacaggtgtacacc |
| | | ctgcccccatcccgggatgagctgaccaagaaccaggtcagcctgacctgcctggtcaaaggcttctatccc |
| | | agcgacatcgccgtggagtgggagagcaatgggcagccggagaacaactacaagaccacgcctcccgtgctg |
| | | gactccgacggctccttcttcctctacagcaagctcaccgtggacaagagcaggtggcagcaggggaacgtc |
| | | ttctcatgctccgtgatgcatgaggctctgcacaaccactacacgcagaagagcctctccctgtctccgggt |
| | | aaa |
| | | SEQ ID NO: 85 |
| | | ------------------------------------------------------------------------ |
| | | -------------------------------------- |
| | | cagtctgccctgactcagcctcgctcagtgtccgcatctcctgggcagtccgtcaccatttcctgcactgga |
| | | acccacaatttggtctcttggtgtcaacatcacccaggcagagcccccaagttattaatttatgacttcaat |
| | | aagaggccctcaggggtccctgatcgcttctctggctccgggtctggcggcacggcctccctaaccatcagt |
| | | ggactccaggatgacgatgacgccgaatatttttgttgggcatatgaagctttcggcggagggaccaagttg |
| | | actgtacttcgtcagcccaaggctgccccctcggtcactctgttcccgccctcctctgaggagcttcaagcc |
| | | aacaaggccacactggtgtgtctcataagtgacttctacccgggagccgtgacagtggcttggaaagcagat |
| | | agcagccccgtcaaggcgggagtggagaccaccacaccctccaaacaaagcaacaacaagtacgcggccagc |
| | | agctatctgagcctgacgcctgagcagtggaagtcccacagaagctacagctgccaggtcacgcatgaaggg |
| | | agcaccgtggagaagacagtggcccctacagaatgttca |
| | | SEQ ID NO: 86 |
| Sequence 6 | N49P18.1 | gcggacttggtgcagtctggggctgtggtgaagaagcctggggactcagtgagaatctcctgtgaggctcaa |
| | | ggatacacattcactgactacgtcattcactggattcgacgggcccctggacaaggccttgaatggatgggg |
| | | tggattaatccaggttatggacaagtaaatattccatggaactttcagggcagggtctccatgacccgagac |
| | | acgtccatcgaaacagcatttctggacttaagaggtctaaagtctgacgacacgggcctctattattgcgtg |
| | | agagatcgaagtaatggatggggaaagcgattcgagtcctccaattggttcctcgatctgtggggccgcggc |
| | | actgtggtcactgttcactcaccctccaccaagggcccatcggtcttccccctggcaccctcctccaagagc |
| | | acctctgggggcacagcggccctgggctgcctggtcaaggactacttccccgaaccggtgacggtgtcgtgg |
| | | aactcaggcgccctgaccagcggcgtgcacaccttcccggctgtcctacagtcctcaggactctactccctc |
| | | agcagcgtggtgaccgtgccctccagcagcttgggcacccagacctacatctgcaacgtgaatcacaagccc |
| | | agcaacaccaaggtggacaagaaagttgagcccaaatcttgtgacaaaactcacacatgcccaccgtgccca |
| | | gcacctgaactcctggggggaccgtcagtcttcctcttccccccaaaacccaaggacaccctcatgatctcc |
| | | cggacccctgaggtcacatgcgtggtggtggacgtgagccacgaagaccctgaggtcaagttcaactggtac |
| | | gtggacggcgtggaggtgcataatgccaagacaaagccgcgggaggagcagtacaacagcacgtaccgggtg |
| | | gtcagcgtcctcaccgtcctgcaccaggactggctgaatggcaaggagtacaagtgcaaggtctccaacaaa |
| | | gccctcccagcccccatcgagaaaaccatctccaaagccaaagggcagccccgagaaccacaggtgtacacc |
| | | ctgcccccatcccgggatgagctgaccaagaaccaggtcagcctgacctgcctggtcaaaggcttctatccc |
| | | agcgacatcgccgtggagtgggagagcaatgggcagccggagaacaactacaagaccacgcctcccgtgctg |
| | | gactccgacggctccttcttcctctacagcaagctcaccgtggacaagagcaggtggcagcaggggaacgtc |
| | | ttctcatgctccgtgatgcatgaggctctgcacaaccactacacgcagaagagcctctccctgtctccgggt |
| | | aaa |
| | | SEQ ID NO: 87 |
| | | ------------------------------------------------------------------------ |
| | | -------------------------------------- |
| | | cagtctgccctgactcagcctcgctcaatgtccgcatctcctgggcagtccgtcaccatttcctgcactgga |
| | | acccacaatttggtctcttggtgtcaacatcacccaggcagaccccccaaattattaatttatgacttcaat |
| | | aagaggccctcaggggtccctgatcgcttctctggctccgggtctggcggcacggcctccctgaccatcagt |
| | | ggactccaggatgacgatgacgccgaatacatttgttgggcatatgaagctttcggcggagggaccaagttg |
| | | accgtacttcgtcagcccaaggctgccccctcggtcactctgttcccgccctcctctgaggagcttcaagcc |
| | | aacaaggccacactggtgtgtctcataagtgacttctacccgggagccgtgacagtggcttggaaagcagat |
| | | agcagccccgtcaaggcgggagtggagaccaccacaccctccaaacaaagcaacaacaagtacgcggccagc |
| | | agctatctgagcctgacgcctgagcagtggaagtcccacagaagctacagctgccaggtcacgcatgaaggg |
| | | agcaccgtggagaagacagtggcccctacagaatgttca |
| | | SEQ ID NO: 88 |
| Sequence 7 | N49P19 | gcggacttggtgcagtctggggctgtggtgaaaaatgctggggcctcagtgagggtctcctgtgaggcttat |
| | | ggatacacattcgtggactacttcattcattgggtccgacaggcccctggacaaggctttgaatggatggga |
| | | tacatggatcccttgaacgggcgcccaaacattgcgcgaaaatttcagggcaggctctccctgagtcgagat |
| | | aggtccagcgaaacttcatttctggacttaagtggactgaggtctgacgactcggccgtctattattgtgtg |
| | | agagacaagagtaatggatcgggcagacggtttgactcgtctaattggtttctcgatctgtggggccgtgga |
| | | acccgggtcagtattttctcagcctccaccaagggcccatcggtcttccccctggcaccctcctccaagagc |
| | | acctctgggggcacagcggccctgggctgcctggtcaaggactacttccccgaaccggtgacggtgtcgtgg |
| | | aactcaggcgccctgaccagcggcgtgcacaccttcccggctgtcctacagtcctcaggactctactccctc |
| | | agcagcgtggtgaccgtgccctccagcagcttgggcacccagacctacatctgcaacgtgaatcacaagccc |
| | | agcaacaccaaggtggacaagaaagttgagcccaaatcttgtgacaaaactcacacatgcccaccgtgccca |
| | | gcacctgaactcctggggggaccgtcagtcttcctcttccccccaaaacccaaggacaccctcatgatctcc |
| | | cggacccctgaggtcacatgcgtggtggtggacgtgagccacgaagaccctgaggtcaagttcaactggtac |
| | | gtggacggcgtggaggtgcataatgccaagacaaagccgcgggaggagcagtacaacagcacgtaccgggtg |
| | | gtcagcgtcctcaccgtcctgcaccaggactggctgaatggcaaggagtacaagtgcaaggtctccaacaaa |
| | | gccctcccagcccccatcgagaaaaccatctccaaagccaaagggcagccccgagaaccacaggtgtacacc |
| | | ctgcccccatcccgggatgagctgaccaagaaccaggtcagcctgacctgcctggtcaaaggcttctatccc |
| | | agcgacatcgccgtggagtgggagagcaatgggcagccggagaacaactacaagaccacgcctcccgtgctg |
| | | gactccgacggctccttcttcctctacagcaagctcaccgtggacaagagcaggtggcagcaggggaacgtc |
| | | ttctcatgctccgtgatgcatgaggctctgcacaaccactacacgcagaagagcctctccctgtctccgggt |
| | | aaa |
| | | SEQ ID NO: 89 |
| | | ------------------------------------------------------------------------ |
| | | -------------------------------------- |
| | | cagtctgccctgactcagcctcgctcagtgtccgcaactcctggacagtcagtcaccatctcctgcactgga |
| | | acccacaattatgtctcttggtgtcaacaacatccaggcagagcccccaaattactaatttatgacttcaat |
| | | aagaggccctcaggggtcccggatcgcttctctggctccggatctggcggcacggcctccctaaccatcact |
| | | ggactccaggatgacgatgaagcggactatttttgttgggcctatgatgcttttggcggagggaccaagttg |
| | | accgtcctgcgtcagcccaaggctgccccctcggtcactctgttcccgccctcctctgaggagcttcaagcc |
| | | aacaaggccacactggtgtgtctcataagtgacttctacccgggagccgtgacagtggcttggaaagcagat |
| | | agcagccccgtcaaggcgggagtggagaccaccacaccctccaaacaaagcaacaacaagtacgcggccagc |
| | | agctatctgagcctgacgcctgagcagtggaagtcccacagaagctacagctgccaggtcacgcatgaaggg |
| | | agcaccgtggagaagacagtggcccctacagaatgttca |
| | | SEQ ID NO: 90 |
| Sequence 8 | N49P37 | gcggacttggtgcagtctggggctgtggtgaagaagcctggggactcagtgagggtctcctgtgaggcttat |
| | | ggatacacattcagtgactacatcattcattggattcgacgggcccctggacgaggccttgaatggatggga |
| | | tggatgaatccgatgggcggacaagtgaatattccgtggaactttcaggggagagtctccatgacccgggac |
| | | acgtccatcgaaacagcatttctggacttaagaggactgaggtctgacgacacggccgtctattactgtgtg |
| | | agagatcgcagcaatggatcgggcaagcgatttgagtcctccaattggttcctcgatctgtggggccgcggg |
| | | accgcggtcactatttcctcaccctccaccaagggcccatcggtcttccccctggcaccctcctccaagagc |
| | | acctctgggggcacagcggccctgggctgcctggtcaaggactacttccccgaaccggtgacggtgtcgtgg |
| | | aactcaggcgccctgaccagcggcgtgcacaccttcccggctgtcctacagtcctcaggactctactccctc |
| | | agcagcgtggtgaccgtgccctccagcagcttgggcacccagacctacatctgcaacgtgaatcacaagccc |
| | | agcaacaccaaggtggacaagaaagttgagcccaaatcttgtgacaaaactcacacatgcccaccgtgccca |
| | | gcacctgaactcctggggggaccgtcagtcttcctcttccccccaaaacccaaggacaccctcatgatctcc |
| | | cggacccctgaggtcacatgcgtggtggtggacgtgagccacgaagaccctgaggtcaagttcaactggtac |
| | | gtggacggcgtggaggtgcataatgccaagacaaagccgcgggaggagcagtacaacagcacgtaccgggtg |
| | | gtcagcgtcctcaccgtcctgcaccaggactggctgaatggcaaggagtacaagtgcaaggtctccaacaaa |
| | | gccctcccagcccccatcgagaaaaccatctccaaagccaaagggcagccccgagaaccacaggtgtacacc |
| | | ctgcccccatcccgggatgagctgaccaagaaccaggtcagcctgacctgcctggtcaaaggcttctatccc |
| | | agcgacatcgccgtggagtgggagagcaatgggcagccggagaacaactacaagaccacgcctcccgtgctg |
| | | gactccgacggctccttcttcctctacagcaagctcaccgtggacaagagcaggtggcagcaggggaacgtc |
| | | ttctcatgctccgtgatgcatgaggctctgcacaaccactacacgcagaagagcctctccctgtctccgggt |
| | | aaa |
| | | SEQ ID NO: 91 |
| | | ------------------------------------------------------------------------ |
| | | -------------------------------------- |
| | | cagtctgccctgactcagcctcgctcagtgtccgcagctcctggacagtccgtcaccatttcctgcactgga |
| | | acccacaatttggtttcttggtgtcaacatcacccaggcagagcccccaagttattaatttatgacttcaat |
| | | aagagaccctcaggtgtccctgatcgtttctctggctccgggtctggcggcacggcctccctaaccatcact |
| | | ggactccaggatgacgatgaggctgaatatttttgttgggcgtatgaagtttttggcggagggaccaagttg |
| | | accgtgcttcgtcagcccaaggctgccccctcggtcactctgttcccgccctcctctgaggagcttcaagcc |
| | | aacaaggccacactggtgtgtctcataagtgacttctacccgggagccgtgacagtggcttggaaagcagat |
| | | agcagccccgtcaaggcgggagtggagaccaccacaccctccaaacaaagcaacaacaagtacgcggccagc |
| | | agctatctgagcctgacgcctgagcagtggaagtcccacagaagctacagctgccaggtcacgcatgaaggg |
| | | agcaccgtggagaagacagtggcccctacagaatgttca |
| | | SEQ ID NO: 92 |
| Sequence 9 | N49P38 | gcggacttggtgcagtctggggctgtggtgaagacgcctggggcctcagtgagggtctcctgtgaggcttat |
| | N49P38.1 | ggatacacattcattgactacatcattcattgggtccgacaggcccctggacaaggttttgaatggctggga |
| | | tacatcgatcctatgaacgggcgcccaaacattgcgcgaaaatttcagggcaggctctccctgagccgggat |
| | | acgtccatcgaaacatcatttctggacttaagtggactgaggtctgacgactcggccgtctattattgtgtg |
| | | agagacaagagtaatggatcgggcaaacgatttgactcctctaattggtttctcgatctgtggggccgtgga |
| | | acgcgggtcagcatttcttcagcctccaccaagggcccatcggtcttccccctggcaccctcctccaagagc |
| | | acctctgggggcacagcggccctgggctgcctggtcaaggactacttccccgaaccggtgacggtgtcgtgg |
| | | aactcaggcgccctgaccagcggcgtgcacaccttcccggctgtcctacagtcctcaggactctactccctc |
| | | agcagcgtggtgaccgtgccctccagcagcttgggcacccagacctacatctgcaacgtgaatcacaagccc |
| | | agcaacaccaaggtggacaagaaagttgagcccaaatcttgtgacaaaactcacacatgcccaccgtgccca |
| | | gcacctgaactcctggggggaccgtcagtcttcctcttccccccaaaacccaaggacaccctcatgatctcc |
| | | cggacccctgaggtcacatgcgtggtggtggacgtgagccacgaagaccctgaggtcaagttcaactggtac |
| | | gtggacggcgtggaggtgcataatgccaagacaaagccgcgggaggagcagtacaacagcacgtaccgggtg |
| | | gtcagcgtcctcaccgtcctgcaccaggactggctgaatggcaaggagtacaagtgcaaggtctccaacaaa |
| | | gccctcccagcccccatcgagaaaaccatctccaaagccaaagggcagccccgagaaccacaggtgtacacc |
| | | ctgcccccatcccgggatgagctgaccaagaaccaggtcagcctgacctgcctggtcaaaggcttctatccc |
| | | agcgacatcgccgtggagtgggagagcaatgggcagccggagaacaactacaagaccacgcctcccgtgctg |
| | | gactccgacggctccttcttcctctacagcaagctcaccgtggacaagagcaggtggcagcaggggaacgtc |
| | | ttctcatgctccgtgatgcatgaggctctgcacaaccactacacgcagaagagcctctccctgtctccgggt |
| | | aaa |
| | | SEQ ID NO: 93 |
| | | ------------------------------------------------------------------------ |
| | | -------------------------------------- |
| | | cagtctgccctgactcagcctcgctcagtgtccgcagctcctggacagtcagtcaccatctcctgcactgga |
| | | acccacaattatgtctcttggtgtcaacaacatccaggcagagcccccaaattactaatttatgacttcaat |
| | | aagaggccctcaggggtcccggatcgcttctctggctccggatctggcggcacggcctccctaaccatcact |
| | | agactccaggatgacgatgacgctgactatttttgttgggcgtatgatgcttttggcggagggaccaagttg |
| | | accgtcctgcgtcagcccaaggctgccccctcggtcactctgttcccgccctcctctgaggagcttcaagcc |
| | | aacaaggccacactggtgtgtctcataagtgacttctacccgggagccgtgacagtggcttggaaagcagat |
| | | agcagccccgtcaaggcgggagtggagaccaccacaccctccaaacaaagcaacaacaagtacgcggccagc |
| | | agctatctgagcctgacgcctgagcagtggaagtcccacagaagctacagctgccaggtcacgcatgaaggg |
| | | agcaccgtggagaagacagtggcccctacagaatgttca |
| | | SEQ ID NO: 94 |
| Sequence 10 | N49P55 | gcggacttggtgcagtctggggctgtggtgaagaagcctggggcctcagtgagggtctcctgtgaggcttat |
| | | ggatacacattcactgactacatcattcattggattcgacaggcccctggacaaggccttgaatggatggga |
| | | tggatgaatcctatgggcgggcgcacaaatattccgtggaaatttcagggcagggtctccatgacccgggac |
| | | acgtccatcgaaacagcatttctggacttaagtggactaacgtctgacgacacggccgtctattattgcgtg |
| | | agagacaagagtaatggatcgggcaaacgatttgactcctctaattggttcctcgatctgtggggccgcgga |
| | | accccggtcactatttcctcaccctccaccaagggcccatcggtcttccccctggcaccctcctccaagagc |
| | | acctctgggggcacagcggccctgggctgcctggtcaaggactacttccccgaaccggtgacggtgtcgtgg |
| | | aactcaggcgccctgaccagcggcgtgcacaccttcccggctgtcctacagtcctcaggactctactccctc |
| | | agcagcgtggtgaccgtgccctccagcagcttgggcacccagacctacatctgcaacgtgaatcacaagccc |
| | | agcaacaccaaggtggacaagaaagttgagcccaaatcttgtgacaaaactcacacatgcccaccgtgccca |
| | | gcacctgaactcctggggggaccgtcagtcttcctcttccccccaaaacccaaggacaccctcatgatctcc |
| | | cggacccctgaggtcacatgcgtggtggtggacgtgagccacgaagaccctgaggtcaagttcaactggtac |
| | | gtggacggcgtggaggtgcataatgccaagacaaagccgcgggaggagcagtacaacagcacgtaccgggtg |
| | | gtcagcgtcctcaccgtcctgcaccaggactggctgaatggcaaggagtacaagtgcaaggtctccaacaaa |
| | | gccctcccagcccccatcgagaaaaccatctccaaagccaaagggcagccccgagaaccacaggtgtacacc |
| | | ctgcccccatcccgggatgagctgaccaagaaccaggtcagcctgacctgcctggtcaaaggcttctatccc |
| | | agcgacatcgccgtggagtgggagagcaatgggcagccggagaacaactacaagaccacgcctcccgtgctg |
| | | gactccgacggctccttcttcctctacagcaagctcaccgtggacaagagcaggtggcagcaggggaacgtc |
| | | ttctcatgctccgtgatgcatgaggctctgcacaaccactacacgcagaagagcctctccctgtctccgggt |
| | | aaa |
| | | SEQ ID NO: 95 |
| | | ------------------------------------------------------------------------ |
| | | -------------------------------------- |
| | | cagtctgccctgactcagcctcgctcagtgtccgcagctcctggacagtccgtcaccatttcctgcactgga |
| | | acccacaatttggtctcttggtgtcaacaacacccaggcagagcccccaaattattaatttatgacttcaat |
| | | aagaggccctcaggggtccctgatcgcttctctggctccgggtctggcggcacggcctccctaagtatcact |
| | | ggactccaggatgacgatgaagctgaatatttttgttgggcgtatgaagcttttggcggagggaccaagttg |
| | | accgtccttcgtcagcccaaggctgccccctcggtcactctgttcccgccctcctctgaggagcttcaagcc |
| | | aacaaggccacactggtgtgtctcataagtgacttctacccgggagccgtgacagtggcttggaaagcagat |
| | | agcagccccgtcaaggcgggagtggagaccaccacaccctccaaacaaagcaacaacaagtacgcggccagc |
| | | agctatctgagcctgacgcctgagcagtggaagtcccacagaagctacagctgccaggtcacgcatgaaggg |
| | | agcaccgtggagaagacagtggcccctacagaatgttca |
| | | SEQ ID NO: 96 |
| Sequence 11 | N49P56 | gcggacttggtgcagtctggggctgtggtgaagaagcctggggcctcagtgcgggtctcctgtgaggcttat |
| | | ggatatacattcgttgactacctcattcattgggtccgacaggcccccggacaaggttttgaatggatggga |
| | | tacatggatcctatgaacgggcgcccaaatattgcgcgaaaatttcagggcaggctctccctgagccgagat |
| | | acgtccatcgaaacatcatttctggacttaagtggactgaggtctgacgactcggccgtctattattgtgtg |
| | | agagacaagagtggtggatcgggcaaactatttgactcctctaattggtttctcgatctgtggggccgtgga |
| | | acccgggtcagcatttcttcagcctccaccaagggcccatcggtcttccccctggcaccctcctccaagagc |
| | | acctctgggggcacagcggccctgggctgcctggtcaaggactacttccccgaaccggtgacggtgtcgtgg |
| | | aactcaggcgccctgaccagcggcgtgcacaccttcccggctgtcctacagtcctcaggactctactccctc |
| | | agcagcgtggtgaccgtgccctccagcagcttgggcacccagacctacatctgcaacgtgaatcacaagccc |
| | | agcaacaccaaggtggacaagaaagttgagcccaaatcttgtgacaaaactcacacatgcccaccgtgccca |
| | | gcacctgaactcctggggggaccgtcagtcttcctcttccccccaaaacccaaggacaccctcatgatctcc |
| | | cggacccctgaggtcacatgcgtggtggtggacgtgagccacgaagaccctgaggtcaagttcaactggtac |
| | | gtggacggcgtggaggtgcataatgccaagacaaagccgcgggaggagcagtacaacagcacgtaccgggtg |
| | | gtcagcgtcctcaccgtcctgcaccaggactggctgaatggcaaggagtacaagtgcaaggtctccaacaaa |
| | | gccctcccagcccccatcgagaaaaccatctccaaagccaaagggcagccccgagaaccacaggtgtacacc |
| | | ctgcccccatcccgggatgagctgaccaagaaccaggtcagcctgacctgcctggtcaaaggcttctatccc |
| | | agcgacatcgccgtggagtgggagagcaatgggcagccggagaacaactacaagaccacgcctcccgtgctg |
| | | gactccgacggctccttcttcctctacagcaagctcaccgtggacaagagcaggtggcagcaggggaacgtc |
| | | ttctcatgctccgtgatgcatgaggctctgcacaaccactacacgcagaagagcctctccctgtctccgggt |
| | | aaa |
| | | SEQ ID NO: 97 |
| | | ------------------------------------------------------------------------ |
| | | -------------------------------------- |
| | | cagtctgccctgactcagcctcgctcagtgtccgcagctcctggacagtcagtcaccatctcctgcaccgga |
| | | actcacaattatgtctcttggtgtcaacaacatccaggcagagcccccaaattactaatttatgacttcaat |
| | | aagaggccctcaggggtcccggatcgcttctctggctccggatctggcggcacggcctccctaaccatcact |
| | | ggactccaggatgacgatgacgctgattatttttgttgggcgtatgatgcttttggcggagggaccaagttg |
| | | accgtcctgcgtcagcccaaggctgccccctcggtcactctgttcccgccctcctctgaggagcttcaagcc |
| | | aacaaggccacactggtgtgtctcataagtgacttctacccgggagccgtgacagtggcttggaaagcagat |
| | | agcagccccgtcaaggcgggagtggagaccaccacaccctccaaacaaagcaacaacaagtacgcggccagc |
| | | agctatctgagcctgacgcctgagcagtggaagtcccacagaagctacagctgccaggtcacgcatgaaggg |
| | | agcaccgtggagaagacagtggcccctacagaatgttca |
| | | SEQ ID NO: 98 |
| Sequence 12 | N49P57 | gcggacttggtgcagtctggggctgtggtgaagaagcctggggactcagtgagaatctcctgtgaggctcaa |
| | | ggatacacattcactgactacgtcattcactggattcgacgggcccctggacaaggccttgaatggatgggg |
| | | tggattaatccaggttatggacaagtaaatattccatggaactttcagggcagggtctccatgacccgagac |
| | | acgtccatcgaaacagcttttctggagttaagaggtctaaagtctgacgacacgggcctctattattgcgtg |
| | | agagatcgaagtaatggatggggaaagcgattcgagtcctccaattggttcctcgatctgtggggccgcggc |
| | | actgtgattactgttcactcaccctccaccaagggcccatcggtcttccccctggcaccctcctccaagagc |
| | | acctctgggggcacagcggccctgggctgcctggtcaaggactacttccccgaaccggtgacggtgtcgtgg |
| | | aactcaggcgccctgaccagcggcgtgcacaccttcccggctgtcctacagtcctcaggactctactccctc |
| | | agcagcgtggtgaccgtgccctccagcagcttgggcacccagacctacatctgcaacgtgaatcacaagccc |
| | | agcaacaccaaggtggacaagaaagttgagcccaaatcttgtgacaaaactcacacatgcccaccgtgccca |
| | | gcacctgaactcctggggggaccgtcagtcttcctcttccccccaaaacccaaggacaccctcatgatctcc |
| | | cggacccctgaggtcacatgcgtggtggtggacgtgagccacgaagaccctgaggtcaagttcaactggtac |
| | | gtggacggcgtggaggtgcataatgccaagacaaagccgcgggaggagcagtacaacagcacgtaccgggtg |
| | | gtcagcgtcctcaccgtcctgcaccaggactggctgaatggcaaggagtacaagtgcaaggtctccaacaaa |
| | | gccctcccagcccccatcgagaaaaccatctccaaagccaaagggcagccccgagaaccacaggtgtacacc |
| | | ctgcccccatcccgggatgagctgaccaagaaccaggtcagcctgacctgcctggtcaaaggcttctatccc |
| | | agcgacatcgccgtggagtgggagagcaatgggcagccggagaacaactacaagaccacgcctcccgtgctg |
| | | gactccgacggctccttcttcctctacagcaagctcaccgtggacaagagcaggtggcagcaggggaacgtc |
| | | ttctcatgctccgtgatgcatgaggctctgcacaaccactacacgcagaagagcctctccctgtctccgggt |
| | | aaa |
| | | SEQ ID NO: 99 |
| | | ------------------------------------------------------------------------ |
| | | -------------------------------------- |
| | | cagtctgccctgactcagcctcgctcaatgtccgcatctcctgggcagtccgtcaccatttcctgcactgga |
| | | acccacaatttggtctcttggtgtcaacatcacccaggcagaccccccaaattattaatttatgacttcaat |
| | | aagaggccctcaggggtccctgatcgcttctctggctccgggtctggcggcacggcctccctgaccatcact |
| | | ggactccaggatgacgatgacgccgaatacatttgttgggcatatgaagctttcggcggagggaccaagttg |
| | | accatacttcgtcagcccaaggctgccccctcggtcactctgttcccgccctcctctgaggagcttcaagcc |
| | | aacaaggccacactggtgtgtctcataagtgacttctacccgggagccgtgacagtggcttggaaagcagat |
| | | agcagccccgtcaaggcgggagtggagaccaccacaccctccaaacaaagcaacaacaagtacgcggccagc |
| | | agctatctgagcctgacgcctgagcagtggaagtcccacagaagctacagctgccaggtcacgcatgaaggg |
| | | agcaccgtggagaagacagtggcccctacagaatgttca |
| | | SEQ ID NO: 100 |
| Sequence 13 | N49P58 | gcggacttggtgcagtctggggctgtggtgaagaagcctggggactcagtgagaatctcctgtgaggctcag |
| | | ggatatacattcaccgactacgtcattcattggattcgacgggcccctggacaaggccttgaatggatgggg |
| | | tggatggatccaagttatggacaagtcaatattccacggaactttcagggcagggtctccatgacccgggac |
| | | acgttcagggaaacagcatatctggaattaagaggtctacagtctgacgacaagggcctctattattgtgtg |
| | | agagatcgaagtcacggatcgggaaggcaattcgagtcctccaactggttcctcgatctgtggggccgcggc |
| | | actgtggtcaatgttcagtcaccctccaccaagggcccatcggtcttccccctggcaccctcctccaagagc |
| | | acctctgggggcacagcggccctgggctgcctggtcaaggactacttccccgaaccggtgacggtgtcgtgg |
| | | aactcaggcgccctgaccagcggcgtgcacaccttcccggctgtcctacagtcctcaggactctactccctc |
| | | agcagcgtggtgaccgtgccctccagcagcttgggcacccagacctacatctgcaacgtgaatcacaagccc |
| | | agcaacaccaaggtggacaagaaagttgagcccaaatcttgtgacaaaactcacacatgcccaccgtgccca |
| | | gcacctgaactcctggggggaccgtcagtcttcctcttccccccaaaacccaaggacaccctcatgatctcc |
| | | cggacccctgaggtcacatgcgtggtggtggacgtgagccacgaagaccctgaggtcaagttcaactggtac |
| | | gtggacggcgtggaggtgcataatgccaagacaaagccgcgggaggagcagtacaacagcacgtaccgggtg |
| | | gtcagcgtcctcaccgtcctgcaccaggactggctgaatggcaaggagtacaagtgcaaggtctccaacaaa |
| | | gccctcccagcccccatcgagaaaaccatctccaaagccaaagggcagccccgagaaccacaggtgtacacc |
| | | ctgcccccatcccgggatgagctgaccaagaaccaggtcagcctgacctgcctggtcaaaggcttctatccc |
| | | agcgacatcgccgtggagtgggagagcaatgggcagccggagaacaactacaagaccacgcctcccgtgctg |
| | | gactccgacggctccttcttcctctacagcaagctcaccgtggacaagagcaggtggcagcaggggaacgtc |
| | | ttctcatgctccgtgatgcatgaggctctgcacaaccactacacgcagaagagcctctccctgtctccgggt |
| | | aaa |
| | | SEQ ID NO: 101 |
| | | ------------------------------------------------------------------------ |
| | | -------------------------------------- |
| | | cagtctgccctgactcagcctcgctcagtgtccgcatctcctgggcagtccgtcaccatttcctgcactgga |
| | | acccacaatttggtctcttggtgtcaacatcacccaggcagacctcccaaattattaatttatgacttcaat |
| | | aagagggcttcaggggtccctgatcgcttctctggctccgggtctggcggcacggcctccctgaccattagt |
| | | ggactccaggatgacgatgacgccgaatatttttgttgggcatatgaagctttcggcggagggaccaagttg |
| | | accgtacttcgtcagcccaaggctgccccctcggtcactctgttcccgccctcctctgaggagcttcaagcc |
| | | aacaaggccacactggtgtgtctcataagtgacttctacccgggagccgtgacagtggcttggaaagcagat |
| | | agcagccccgtcaaggcgggagtggagaccaccacaccctccaaacaaagcaacaacaagtacgcggccagc |
| | | agctatctgagcctgacgcctgagcagtggaagtcccacagaagctacagctgccaggtcacgcatgaaggg |
| | | agcaccgtggagaagacagtggcccctacagaatgttca |
| | | SEQ ID NO: 102 |
| Sequence 14 | N49P59 | gcggacttggtgcagtctggggctgtggtgaagaagcctggggactcactgagaatctcctgtgaggctcaa |
| | | ggatacacattcactgactacgtcattcactggattcgacgggcccctggacaaggccttgaatggatggga |
| | | tggatggatccaagttttggacaaatgaacattccacggaactttcagggcagggtctccatgacccgtgac |
| | | atgtacatcgaaacagcatttctggacttaagaggtctaaagtctgacgacacgggcctctattattgcgtg |
| | | agagatcgaagtcatggatcgggaaggctattcgagtcctccaattggttcctcgatctgtggggccgcggc |
| | | actgtggtcactgttcagtcaccctccaccaagggcccatcggtcttccccctggcaccctcctccaagagc |
| | | acctctgggggcacagcggccctgggctgcctggtcaaggactacttccccgaaccggtgacggtgtcgtgg |
| | | aactcaggcgccctgaccagcggcgtgcacaccttcccggctgtcctacagtcctcaggactctactccctc |
| | | agcagcgtggtgaccgtgccctccagcagcttgggcacccagacctacatctgcaacgtgaatcacaagccc |
| | | agcaacaccaaggtggacaagaaagttgagcccaaatcttgtgacaaaactcacacatgcccaccgtgccca |
| | | gcacctgaactcctggggggaccgtcagtcttcctcttccccccaaaacccaaggacaccctcatgatctcc |
| | | cggacccctgaggtcacatgcgtggtggtggacgtgagccacgaagaccctgaggtcaagttcaactggtac |
| | | gtggacggcgtggaggtgcataatgccaagacaaagccgcgggaggagcagtacaacagcacgtaccgggtg |
| | | gtcagcgtcctcaccgtcctgcaccaggactggctgaatggcaaggagtacaagtgcaaggtctccaacaaa |
| | | gccctcccagcccccatcgagaaaaccatctccaaagccaaagggcagccccgagaaccacaggtgtacacc |
| | | ctgcccccatcccgggatgagctgaccaagaaccaggtcagcctgacctgcctggtcaaaggcttctatccc |
| | | agcgacatcgccgtggagtgggagagcaatgggcagccggagaacaactacaagaccacgcctcccgtgctg |
| | | gactccgacggctccttcttcctctacagcaagctcaccgtggacaagagcaggtggcagcaggggaacgtc |
| | | ttctcatgctccgtgatgcatgaggctctgcacaaccactacacgcagaagagcctctccctgtctccgggt |
| | | aaa |
| | | SEQ ID NO: 103 |
| | | ------------------------------------------------------------------------ |
| | | -------------------------------------- |
| | | cagtctgccctgactcagcctcgctcagtgtccgcatctcctgggcagtccgtcaccatttcctgcactgga |
| | | acccacaatttggtctcttggtgtcaacatcacccaggcagacctcccaaattattaatttatgacttcaat |
| | | aagagggcttcaggggtccctgatcgcttctctggctccgggtctggcggcacggcctccctgaccattagt |
| | | ggactccaggatgacgatgacgccgaatatttttgttgggcatatgaagctttcggcggagggaccaagttg |
| | | accgtacttcgtcagcccaaggctgccccctcggtcactctgttcccgccctcctctgaggagcttcaagcc |
| | | aacaaggccacactggtgtgtctcataagtgacttctacccgggagccgtgacagtggcttggaaagcagat |
| | | agcagccccgtcaaggcgggagtggagaccaccacaccctccaaacaaagcaacaacaagtacgcggccagc |
| | | agctatctgagcctgacgcctgagcagtggaagtcccacagaagctacagctgccaggtcacgcatgaaggg |
| | | agcaccgtggagaagacagtggcccctacagaatgttca |
| | | SEQ ID NO: 104 |
| Sequence 15 | N49P73 | gcggacttggtgcagtctggggctgtggtgaagaagcctggggactcagtgagaatctcctgtgaggctcaa |
| | | ggatacagattcactgactacgtcattcattggattcgacgggcccctggacaaggccttgaatggatgggg |
| | | ttgatggatccaagttttggacgaatgaatattccacggaaatttcagggcagggtctccatgacccgggac |
| | | acgtccatggaaacagcatttctggacttcagaggtctaaattttgacgacacgggcctctattattgcgtg |
| | | agagatcgaagtcatggatcgggaagactattcgagtcctccaattggttcctcgatctgtggggccgcggc |
| | | actgtggtcactgttcagtcaccctccaccaagggcccatcggtcttccccctggcaccctcctccaagagc |
| | | acctctgggggcacagcggccctgggctgcctggtcaaggactacttccccgaaccggtgacggtgtcgtgg |
| | | aactcaggcgccctgaccagcggcgtgcacaccttcccggctgtcctacagtcctcaggactctactccctc |
| | | agcagcgtggtgaccgtgccctccagcagcttgggcacccagacctacatctgcaacgtgaatcacaagccc |
| | | agcaacaccaaggtggacaagaaagttgagcccaaatcttgtgacaaaactcacacatgcccaccgtgccca |
| | | gcacctgaactcctggggggaccgtcagtcttcctcttccccccaaaacccaaggacaccctcatgatctcc |
| | | cggacccctgaggtcacatgcgtggtggtggacgtgagccacgaagaccctgaggtcaagttcaactggtac |
| | | gtggacggcgtggaggtgcataatgccaagacaaagccgcgggaggagcagtacaacagcacgtaccgggtg |
| | | gtcagcgtcctcaccgtcctgcaccaggactggctgaatggcaaggagtacaagtgcaaggtctccaacaaa |
| | | gccctcccagcccccatcgagaaaaccatctccaaagccaaagggcagccccgagaaccacaggtgtacacc |
| | | ctgcccccatcccgggatgagctgaccaagaaccaggtcagcctgacctgcctggtcaaaggcttctatccc |
| | | agcgacatcgccgtggagtgggagagcaatgggcagccggagaacaactacaagaccacgcctcccgtgctg |
| | | gactccgacggctccttcttcctctacagcaagctcaccgtggacaagagcaggtggcagcaggggaacgtc |
| | | ttctcatgctccgtgatgcatgaggctctgcacaaccactacacgcagaagagcctctccctgtctccgggt |
| | | aaa |
| | | SEQ ID NO: 105 |
| | | ------------------------------------------------------------------------ |
| | | -------------------------------------- |
| | | cagtctgccctgactcagcctcgctcagtgtccgcatctcctgggcagtccgtcaccatttcctgcactgga |
| | | acccacaatttggtctcttggtgtcaacatcacccaggcagacctcccaaattattaatttatgacttcaat |
| | | aagagggcatcaggggtccctgatcgcttctctggctccgggtctggcggcacggcctccctgaccatcagt |
| | | ggactccaagatgacgatgacgccgaatatttttgttgggcatatgaagctttcggcggagggaccaagttg |
| | | accgtacttcgtcagcccaaggctgccccctcggtcactctgttcccgccctcctctgaggagcttcaagcc |
| | | aacaaggccacactggtgtgtctcataagtgacttctacccgggagccgtgacagtggcttggaaagcagat |
| | | agcagccccgtcaaggcgggagtggagaccaccacaccctccaaacaaagcaacaacaagtacgcggccagc |
| | | agctatctgagcctgacgcctgagcagtggaagtcccacagaagctacagctgccaggtcacgcatgaaggg |
| | | agcaccgtggagaagacagtggcccctacagaatgttca |
| | | SEQ ID NO: 106 |
| Sequence 16 | N49P74 | ccggacttggtgcagtctggggctgtggtgaagaagcctggggactcagtgagaatttcctgtgaggctcaa |
| | | ggatacacattcattgactacgtcattcactggattcgacgggcccctggacaaggccttgaatggatgggg |
| | | ttgatggatccaacttatggacgaatgaatattccacggaagtttcagggcagggtctccatgacccgggac |
| | | acgtccatcgaaacagcatttctggacttaagaggtctaaaatctgacgacacgggcctctattattgcgtg |
| | | agagatcgaagtcatggatcgggaaggctattcgagtcctccaactggttcctggatctgtggggccgcggc |
| | | actgtggtcactgttcagtcaccctccaccaagggcccatcggtcttccccctggcaccctcctccaagagc |
| | | acctctgggggcacagcggccctgggctgcctggtcaaggactacttccccgaaccggtgacggtgtcgtgg |
| | | aactcaggcgccctgaccagcggcgtgcacaccttcccggctgtcctacagtcctcaggactctactccctc |
| | | agcagcgtggtgaccgtgccctccagcagcttgggcacccagacctacatctgcaacgtgaatcacaagccc |
| | | agcaacaccaaggtggacaagaaagttgagcccaaatcttgtgacaaaactcacacatgcccaccgtgccca |
| | | gcacctgaactcctggggggaccgtcagtcttcctcttccccccaaaacccaaggacaccctcatgatctcc |
| | | cggacccctgaggtcacatgcgtggtggtggacgtgagccacgaagaccctgaggtcaagttcaactggtac |
| | | gtggacggcgtggaggtgcataatgccaagacaaagccgcgggaggagcagtacaacagcacgtaccgggtg |
| | | gtcagcgtcctcaccgtcctgcaccaggactggctgaatggcaaggagtacaagtgcaaggtctccaacaaa |
| | | gccctcccagcccccatcgagaaaaccatctccaaagccaaagggcagccccgagaaccacaggtgtacacc |
| | | ctgcccccatcccgggatgagctgaccaagaaccaggtcagcctgacctgcctggtcaaaggcttctatccc |
| | | agcgacatcgccgtggagtgggagagcaatgggcagccggagaacaactacaagaccacgcctcccgtgctg |
| | | gactccgacggctccttcttcctctacagcaagctcaccgtggacaagagcaggtggcagcaggggaacgtc |
| | | ttctcatgctccgtgatgcatgaggctctgcacaaccactacacgcagaagagcctctccctgtctccgggt |
| | | aaa |
| | | SEQ ID NO: 107 |
| | | ------------------------------------------------------------------------ |
| | | -------------------------------------- |
| | | cagtctgccctgactcagcctcgctcagtgtccgcatctcctgggcagtccgtcaccatttcctgcactgga |
| | | acccacaatttggtctcttggtgtcaacatcacccaggcagacctcccaaattattaatttatgacttcaat |
| | | aagagggcttcaggggtccctgatcgcttctctggctccgggtctggcggcacggcctccctgaccatcagt |
| | | ggactccaagatgacgatgacgccgaatatttttgttgggcatatgaagctttcggcggagggaccaagttg |
| | | accgtacttcgtcagcccaaggctgccccctcggtcactctgttcccgccctcctctgaggagcttcaagcc |
| | | aacaaggccacactggtgtgtctcataagtgacttctacccgggagccgtgacagtggcttggaaagcagat |
| | | agcagccccgtcaaggcgggagtggagaccaccacaccctccaaacaaagcaacaacaagtacgcggccagc |
| | | agctatctgagcctgacgcctgagcagtggaagtcccacagaagctacagctgccaggtcacgcatgaaggg |
| | | agcaccgtggagaagacagtggcccctacagaatgttca |
| | | SEQ ID NO: 108 |
| Sequence 17 | N49P75 | gcggacttggtgcagtctggggctgtggtgaagaagcctggggactcagtgaggatctcctgtgaggctcaa |
| | | ggatacagatttcttgactacatcattcactggattcgacgggcccctggacaaggccttgaatggatggga |
| | | tggatgaatccaatgggcggacaagtaaacattccatggaactttcagggtagggtctccatgacccgggac |
| | | acgtccatcgaaacagcatttctggacttaagaggactaaagtctgacgacacggccgtctattattgcgtg |
| | | agagatcgcagtaatggatcgggaaagcgattcgagtcctccaattggttcctcgatctgtggggccgcggg |
| | | actgcggtcactattcattcaccctccaccaagggcccatcggtcttccccctggcaccctcctccaagagc |
| | | acctctgggggcacagcggccctgggctgcctggtcaaggactacttccccgaaccggtgacggtgtcgtgg |
| | | aactcaggcgccctgaccagcggcgtgcacaccttcccggctgtcctacagtcctcaggactctactccctc |
| | | agcagcgtggtgaccgtgccctccagcagcttgggcacccagacctacatctgcaacgtgaatcacaagccc |
| | | agcaacaccaaggtggacaagaaagttgagcccaaatcttgtgacaaaactcacacatgcccaccgtgccca |
| | | gcacctgaactcctggggggaccgtcagtcttcctcttccccccaaaacccaaggacaccctcatgatctcc |
| | | cggacccctgaggtcacatgcgtggtggtggacgtgagccacgaagaccctgaggtcaagttcaactggtac |
| | | gtggacggcgtggaggtgcataatgccaagacaaagccgcgggaggagcagtacaacagcacgtaccgggtg |
| | | gtcagcgtcctcaccgtcctgcaccaggactggctgaatggcaaggagtacaagtgcaaggtctccaacaaa |
| | | gccctcccagcccccatcgagaaaaccatctccaaagccaaagggcagccccgagaaccacaggtgtacacc |
| | | ctgcccccatcccgggatgagctgaccaagaaccaggtcagcctgacctgcctggtcaaaggcttctatccc |
| | | agcgacatcgccgtggagtgggagagcaatgggcagccggagaacaactacaagaccacgcctcccgtgctg |
| | | gactccgacggctccttcttcctctacagcaagctcaccgtggacaagagcaggtggcagcaggggaacgtc |
| | | ttctcatgctccgtgatgcatgaggctctgcacaaccactacacgcagaagagcctctccctgtctccgggt |
| | | aaa |
| | | SEQ ID NO: 109 |
| | | ------------------------------------------------------------------------ |
| | | -------------------------------------- |
| | | cagtctgccctgactcagcctcgctcagtgtccgcatctcctgggcagtccgtcaccatttcctgcactgga |
| | | acccacaatttggtctcttggtgtcaacatcacccaggcagagcccccaaattattaatttatgacttcaat |
| | | aagaggccctcaggggtccctgatcgcttctctggctccgggtctggcggcacggcctccctgaccatcact |
| | | ggactccaggatgacgatgacgccgaatatttttgttgggcgtatgaagcttttggcggagggaccaagttg |
| | | accgtacttcgtcagcccaaggctgccccctcggtcactctgttcccgccctcctctgaggagcttcaagcc |
| | | aacaaggccacactggtgtgtctcataagtgacttctacccgggagccgtgacagtggcttggaaagcagat |
| | | agcagccccgtcaaggcgggagtggagaccaccacaccctccaaacaaagcaacaacaagtacgcggccagc |
| | | agctatctgagcctgacgcctgagcagtggaagtcccacagaagctacagctgccaggtcacgcatgaaggg |
| | | agcaccgtggagaagacagtggcccctacagaatgttca |
| | | SEQ ID NO: 110 |
| Sequence 18 | N49P9 | cacgtccaattggtgcagtctggaggtggggtgaagaagattggggccgctgtgaggatctcctgcgaggtg |
| | N49P9.1 | actggatataaattcatggaccaactcataaactgggtgcggcaggcccccggtcagggccttgagtggatg |
| | N49P9.2 | ggatggatgaatccaacatatggacaagtaaattattcatggagatttgaaggaagggtcaccatgaccagg |
| | N49P9i7 | gacatggacaccgagacggccttcatggagttgagaggactgagagtggacgacacggccgtctattattgc |
| | N49P9-FR | gcgagggggccctctggggaaaattatccttttcactattggggccagggtgtccgagtggtcgtctcgtca |
| | | ccctccaccaagggcccatcggtcttccccctggcaccctcctccaagagcacctctgggggcacagcggcc |
| | | ctgggctgcctggtcaaggactacttccccgaaccggtgacggtgtcgtggaactcaggcgccctgaccagc |
| | | ggcgtgcacaccttcccggctgtcctacagtcctcaggactctactccctcagcagcgtggtgaccgtgccc |
| | | tccagcagcttgggcacccagacctacatctgcaacgtgaatcacaagcccagcaacaccaaggtggacaag |
| | | aaagttgagcccaaatcttgtgacaaaactcacacatgcccaccgtgcccagcacctgaactcctgggggga |
| | | ccgtcagtcttcctcttccccccaaaacccaaggacaccctcatgatctcccggacccctgaggtcacatgc |
| | | gtggtggtggacgtgagccacgaagaccctgaggtcaagttcaactggtacgtggacggcgtggaggtgcat |
| | | aatgccaagacaaagccgcgggaggagcagtacaacagcacgtaccgggtggtcagcgtcctcaccgtcctg |
| | | caccaggactggctgaatggcaaggagtacaagtgcaaggtctccaacaaagccctcccagcccccatcgag |
| | | aaaaccatctccaaagccaaagggcagccccgagaaccacaggtgtacaccctgcccccatcccgggatgag |
| | | ctgaccaagaaccaggtcagcctgacctgcctggtcaaaggcttctatcccagcgacatcgccgtggagtgg |
| | | gagagcaatgggcagccggagaacaactacaagaccacgcctcccgtgctggactccgacggctccttcttc |
| | | ctctacagcaagctcaccgtggacaagagcaggtggcagcaggggaacgtcttctcatgctccgtgatgcat |
| | | gaggctctgcacaaccactacacgcagaagagcctctccctgtctccgggtaaa |
| | | SEQ ID NO: 111 |
| | | ------------------------------------------------------------------------ |
| | | -------------------------------------- |
| | | gcatctgccctgactcagcctgcctccatgtctgcgtcccctggacagtcggtaaccatctcgtgctctgga |
| | | accagacacataatctctgcttggttccaacaatatccaggcaaaccacccaaactcataatttttgacgac |
| | | gataagcgtccctctggagttcctagtcgcttctctgcctccaggcctggcgacacggcctccctgacaatc |
| | | tctaatgttcaacctgaggacgaggcgacgtacatttgcaatacatatgaattctttggcggagggaccaga |
| | | ttgaccgtcctaagtcagcccaaggctgccccctcggtcactctgttcccaccctcctctgaggagcttcaa |
| | | gccaacaaggccacactggtgtgtctcgtaagtgacttctacccgggagccgtgacagtggcctggaaggca |
| | | gatggcagccccgtcaaggtgggagtggagaccaccaaaccctccaaacaaagcaacaacaagtatgcggcc |
| | | agcagctacctgagcctgacgcccgagcagtggaagtcccacagaagctacagctgccgggtcacgcatgaa |
| | | gggagcaccgtggagaagacagtggcccctgcagaatgctct |
| | | SEQ ID NO: 112 |
| Sequence 19 | N49P22 | cacattcagttgctacagtcggggcctcaagttaagaagtctggggacacagtgagaatctcctgtgagacc |
| | | tctggatataacttcgtcgactcccgtatccactgggtccgacagaccccggaaaaacgtctcagatggatg |
| | | ggctggatcaatcctctccaaggtggtgtgaattacgcgccggaatttcagggcagaatcaggatgaccagg |
| | | gacacatttatagacacagtttacgtggacctgagcggactgacaccggccgacacggcctattattactgc |
| | | gcgcgagggatcgatggcaagtcttacccctttcatttctggggccacggaacccgggtcaccgtcttctcg |
| | | gcctccaccaagggcccatcggtcttccccctggcaccctcctccaagagcacctctgggggcacagcggcc |
| | | ctgggctgcctggtcaaggactacttccccgaaccggtgacggtgtcgtggaactcaggcgccctgaccagc |
| | | ggcgtgcacaccttcccggctgtcctacagtcctcaggactctactccctcagcagcgtggtgaccgtgccc |
| | | tccagcagcttgggcacccagacctacatctgcaacgtgaatcacaagcccagcaacaccaaggtggacaag |
| | | aaagttgagcccaaatcttgtgacaaaactcacacatgcccaccgtgcccagcacctgaactcctgggggga |
| | | ccgtcagtcttcctcttccccccaaaacccaaggacaccctcatgatctcccggacccctgaggtcacatgc |
| | | gtggtggtggacgtgagccacgaagaccctgaggtcaagttcaactggtacgtggacggcgtggaggtgcat |
| | | aatgccaagacaaagccgcgggaggagcagtacaacagcacgtaccgggtggtcagcgtcctcaccgtcctg |
| | | caccaggactggctgaatggcaaggagtacaagtgcaaggtctccaacaaagccctcccagcccccatcgag |
| | | aaaaccatctccaaagccaaagggcagccccgagaaccacaggtgtacaccctgcccccatcccgggatgag |
| | | ctgaccaagaaccaggtcagcctgacctgcctggtcaaaggcttctatcccagcgacatcgccgtggagtgg |
| | | gagagcaatgggcagccggagaacaactacaagaccacgcctcccgtgctggactccgacggctccttcttc |
| | | ctctacagcaagctcaccgtggacaagagcaggtggcagcaggggaacgtcttctcatgctccgtgatgcat |
| | | gaggctctgcacaaccactacacgcagaagagcctctccctgtctccgggtaaa |
| | | SEQ ID NO: 113 |
| | | ------------------------------------------------------------------------ |
| | | -------------------------------------- |
| | | cgatttgccctgactcaacctgcctccgtgtctgggtctcctggacagacgatcaccataacctgcgctgga |
| | | ggcagcgtctcctggtttcatttccctccaggcaaaacccccagactcattatttatgagtcttctaagcga |
| | | ccctctggggtctctcctcgattctctgggtcccagtctggcagcacggcctcccttataatttctggcctc |
| | | cagtctgatgacgaagggacatacttctgttctattcttgaatttttcggcagagggactcttgtcaccgtc |
| | | ctgagtcagcccaaggctgccccctcggtcactctgttcccaccctcctctgaggagcttcaagccaacaag |
| | | gccacactggtgtgtctcgtaagtgacttctacccgggagccgtgacagtggcctggaaggcagatggcagc |
| | | cccgtcaaggtgggagtggagaccaccaaaccctccaaacaaagcaacaacaagtatgcggccagcagctac |
| | | ctgagcctgacgcccgagcagtggaagtcccacagaagctacagctgccgggtcacgcatgaagggagcacc |
| | | gtggagaagacagtggcccctgcagaatgctct |
| | | SEQ ID NO: 114 |
| Sequence 20 | N49P23 | caggtgcgcttggtgcagtctggggctggggcgaggaagactggggcctcaatgaaactttcctgctcgacc |
| | | tctggatacaccttcaccactcatcacggccacttcataaattgggtgcgacaggcccgtggacaagggctt |
| | | gagtggatggggtggatgaatcccatgactgggcagatgaatattgaggggaaatttcagggcagagtcacc |
| | | ctcactcgagacatatacagtgacacggcttacatggaaatgaccagactgacaactggcgacacgggcact |
| | | tattactgtgcgcgaggcgatttcggacagaattatccctttcattattggggccagggaagcctggtcatc |
| | | gtctcctcggcctccaccaagggcccatcggtcttccccctggcaccctcctccaagagcacctctgggggc |
| | | acagcggccctgggctgcctggtcaaggactacttccccgaaccggtgacggtgtcgtggaactcaggcgcc |
| | | ctgaccagcggcgtgcacaccttcccggctgtcctacagtcctcaggactctactccctcagcagcgtggtg |
| | | accgtgccctccagcagcttgggcacccagacctacatctgcaacgtgaatcacaagcccagcaacaccaag |
| | | gtggacaagaaagttgagcccaaatcttgtgacaaaactcacacatgcccaccgtgcccagcacctgaactc |
| | | ctggggggaccgtcagtcttcctcttccccccaaaacccaaggacaccctcatgatctcccggacccctgag |
| | | gtcacatgcgtggtggtggacgtgagccacgaagaccctgaggtcaagttcaactggtacgtggacggcgtg |
| | | gaggtgcataatgccaagacaaagccgcgggaggagcagtacaacagcacgtaccgggtggtcagcgtcctc |
| | | accgtcctgcaccaggactggctgaatggcaaggagtacaagtgcaaggtctccaacaaagccctcccagcc |
| | | cccatcgagaaaaccatctccaaagccaaagggcagccccgagaaccacaggtgtacaccctgcccccatcc |
| | | cgggatgagctgaccaagaaccaggtcagcctgacctgcctggtcaaaggcttctatcccagcgacatcgcc |
| | | gtggagtgggagagcaatgggcagccggagaacaactacaagaccacgcctcccgtgctggactccgacggc |
| | | tccttcttcctctacagcaagctcaccgtggacaagagcaggtggcagcaggggaacgtcttctcatgctcc |
| | | gtgatgcatgaggctctgcacaaccactacacgcagaagagcctctccctgtctccgggtaaa |
| | | SEQ ID NO: 115 |
| | | ------------------------------------------------------------------------ |
| | | -------------------------------------- |
| | | ctgtctgccctgactcagcctgcctccgtgtctgggtctcctgggcagtcggtcaccatctcctgctctgga |
| | | acgaaccgttaccttgtctcctggtatcaacaacaccctgacaaagcccccaaactcatcatttatgacgac |
| | | aataagcggccctcaggaatttctgatcgcttctcagcctccaggcctgacgacacggcctccctgacaatc |
| | | tctggactccagactggggacgaggctacttattggtgtgcctcatatgaacgttttggcggcgggacgagg |
| | | ctgaccgtccttagtcagcccaaggctgccccctcggtcactctgttcccaccctcctctgaggagcttcaa |
| | | gccaacaaggccacactggtgtgtctcgtaagtgacttctacccgggagccgtgacagtggcctggaaggca |
| | | gatggcagccccgtcaaggtgggagtggagaccaccaaaccctccaaacaaagcaacaacaagtatgcggcc |
| | | agcagctacctgagcctgacgcccgagcagtggaagtcccacagaagctacagctgccgggtcacgcatgaa |
| | | gggagcaccgtggagaagacagtggcccctgcagaatgctct |
| | | SEQ ID NO: 116 |
| Sequence 21 | N49P9.3 | cacgtccaattggtgcagtctggaggtggggtgaagaagattggggccgctgtgaggatctcctgcgaggtg |
| | N49P9.4 | tctggatacaacttcatggaccaattcataaattgggtgcgacaggcccccggtcagggccttgagtggatg |
| | N49P9.6 | ggatggatgaacccaatatatggacaagtaaattattcatggagatttcaaggaagggtcaccatgaccagg |
| | N49P9.3-FR | gacatgtacaccgacacggccttcatggagttgagaggactgagagtggacgacacggccgtctattattgc |
| | N49P9.6-FR | gcgagggggccctctggggaaaattatccttttcactattggggccagggtgtccgagtggtcgtctcgtca |
| | N49P9.6- | ccctccaccaagggcccatcggtcttccccctggcaccctcctccaagagcacctctgggggcacagcggcc |
| | FR-54W | ctgggctgcctggtcaaggactacttccccgaaccggtgacggtgtcgtggaactcaggcgccctgaccagc |
| | N49P9.6- | ggcgtgcacaccttcccggctgtcctacagtcctcaggactctactccctcagcagcgtggtgaccgtgccc |
| | FR-54-F | tccagcagcttgggcacccagacctacatctgcaacgtgaatcacaagcccagcaacaccaaggtggacaag |
| | N49P9.6- | aaagttgagcccaaatcttgtgacaaaactcacacatgcccaccgtgcccagcacctgaactcctgggggga |
| | 54W | ccgtcagtcttcctcttccccccaaaacccaaggacaccctcatgatctcccggacccctgaggtcacatgc |
| | N49P9.6- | gtggtggtggacgtgagccacgaagaccctgaggtcaagttcaactggtacgtggacggcgtggaggtgcat |
| | 54F | aatgccaagacaaagccgcgggaggagcagtacaacagcacgtaccgggtggtcagcgtcctcaccgtcctg |
| | N49P9.6- | caccaggactggctgaatggcaaggagtacaagtgcaaggtctccaacaaagccctcccagcccccatcgag |
| | FR3-06 | aaaaccatctccaaagccaaagggcagccccgagaaccacaggtgtacaccctgcccccatcccgggatgag |
| | N49P9.6- | ctgaccaagaaccaggtcagcctgacctgcctggtcaaaggcttctatcccagcgacatcgccgtggagtgg |
| | FR1-D | gagagcaatgggcagccggagaacaactacaagaccacgcctcccgtgctggactccgacggctccttcttc |
| | N49P9.6- | ctctacagcaagctcaccgtggacaagagcaggtggcagcaggggaacgtcttctcatgctccgtgatgcat |
| | FR1-D-I | gaggctctgcacaaccactacacgcagaagagcctctccctgtctccgggtaaa |
| | | SEQ ID NO: 117 |
| | | ------------------------------------------------------------------------ |
| | | -------------------------------------- |
| | | ctgactcagcctgcctccatgtctgcgtcccctggacagtcggtaaccatctcgtgctctggaaccagacac |
| | | ataatctctgcttggttccaacaatatccaggcaaaccacccaaactcataatttttgacgacgataagcgt |
| | | ccctctggagttcctagtcgcttctctgcctccaggcctggcgacacggcctccctgacaatctctaatgtt |
| | | caacctgaggacgaggcgacatacatttgcaatacatatgaattctttggcggagggaccaaattgaccgtc |
| | | ctaagtcagcccaaggctgccccctcggtcactctgttcccaccctcctctgaggagcttcaagccaacaag |
| | | gccacactggtgtgtctcgtaagtgacttctacccgggagccgtgacagtggcctggaaggcagatggcagc |
| | | cccgtcaaggtgggagtggagaccaccaaaccctccaaacaaagcaacaacaagtatgcggccagcagctac |
| | | ctgagcctgacgcccgagcagtggaagtcccacagaagctacagctgccgggtcacgcatgaagggagcacc |
| | | gtggagaagacagtggcccctgcagaatgctct |
| | | SEQ ID NO: 118 |
| Sequence 22 | N49P51 | cacattcagttgctacagtcggggcctcaagttaagaagtctggggacacagtgagaatctcctgtgagacc |
| | | tctggatataacttcgtcgactcccgtatccactgggtccgacagaccccggaaaaacgtctcagatggatg |
| | | ggctggatcaatcctctccacggtggtgtgaattacgcgccggaatttcagggcagaatcaggatgaccagg |
| | | gacacatttatagacacagtttacgtggacctgagcggactgacaccggccgacacggcctattattactgc |
| | | gcgcgagggatcgatggcaagtcttacccctttcatttctggggccacggaacccgggtcaccgtcttctcg |
| | | gcctccaccaagggcccatcggtcttccccctggcaccctcctccaagagcacctctgggggcacagcggcc |
| | | ctgggctgcctggtcaaggactacttccccgaaccggtgacggtgtcgtggaactcaggcgccctgaccagc |
| | | ggcgtgcacaccttcccggctgtcctacagtcctcaggactctactccctcagcagcgtggtgaccgtgccc |
| | | tccagcagcttgggcacccagacctacatctgcaacgtgaatcacaagcccagcaacaccaaggtggacaag |
| | | aaagttgagcccaaatcttgtgacaaaactcacacatgcccaccgtgcccagcacctgaactcctgggggga |
| | | ccgtcagtcttcctcttccccccaaaacccaaggacaccctcatgatctcccggacccctgaggtcacatgc |
| | | gtggtggtggacgtgagccacgaagaccctgaggtcaagttcaactggtacgtggacggcgtggaggtgcat |
| | | aatgccaagacaaagccgcgggaggagcagtacaacagcacgtaccgggtggtcagcgtcctcaccgtcctg |
| | | caccaggactggctgaatggcaaggagtacaagtgcaaggtctccaacaaagccctcccagcccccatcgag |
| | | aaaaccatctccaaagccaaagggcagccccgagaaccacaggtgtacaccctgcccccatcccgggatgag |
| | | ctgaccaagaaccaggtcagcctgacctgcctggtcaaaggcttctatcccagcgacatcgccgtggagtgg |
| | | gagagcaatgggcagccggagaacaactacaagaccacgcctcccgtgctggactccgacggctccttcttc |
| | | ctctacagcaagctcaccgtggacaagagcaggtggcagcaggggaacgtcttctcatgctccgtgatgcat |
| | | gaggctctgcacaaccactacacgcagaagagcctctccctgtctccgggtaaa |
| | | SEQ ID NO: 119 |
| | | ------------------------------------------------------------------------ |
| | | -------------------------------------- |
| | | cgatttgccctgactcaacctgcctccgtgtctgggtctcctggacagacgatcaccataacctgcgctgga |
| | | ggcagcgtctcctggtttcatttccctccaggcaaaacccccagactcattatttatgagtcttctaagcga |
| | | ccctctggggtctctcctcgattctctgggtcccagtctggcagcacggcctccctcataatttctggcctc |
| | | cagtctgatgacgaagggacatacttctgttctattcttgaatttttcggcagagggactcttgtcaccgtc |
| | | ctgagtcagcccaaggctgccccctcggtcactctgttcccaccctcctctgaggagcttcaagccaacaag |
| | | gccacactggtgtgtctcgtaagtgacttctacccgggagccgtgacagtggcctggaaggcagatggcagc |
| | | cccgtcaaggtgggagtggagaccaccaaaccctccaaacaaagcaacaacaagtatgcggccagcagctac |
| | | ctgagcctgacgcccgagcagtggaagtcccacagaagctacagctgccgggtcacgcatgaagggagcacc |
| | | gtggagaagacagtggcccctgcagaatgctct |
| | | SEQ ID NO: 120 |
| Sequence 23 | N49P52 | cgtgttacattacaacaatctggggctatagtgaggcagcctggggcctcagtgaccgtctcctgcgagact |
| | | tctggatatactttcaccaagtatttcatctactgggtgcgacaggcccctggacagggtcttgagtggctg |
| | | ggcagaatacacccccgaaccggtgccgtgaagtatgcaccgagatttcagggtagactgtccatgaccaga |
| | | gactggtcactcgacacagcctacctcggattgaccggactgacactcggcgacacggctctatatttctgt |
| | | gcgaggggggcctttgaggcagattcatatgggtcaagttatccctttcaccactggggccagggaacccta |
| | | gtcaccgtctccgcggcctccaccaagggcccatcggtcttccccctggcaccctcctccaagagcacctct |
| | | gggggcacagcggccctgggctgcctggtcaaggactacttccccgaaccggtgacggtgtcgtggaactca |
| | | ggcgccctgaccagcggcgtgcacaccttcccggctgtcctacagtcctcaggactctactccctcagcagc |
| | | gtggtgaccgtgccctccagcagcttgggcacccagacctacatctgcaacgtgaatcacaagcccagcaac |
| | | accaaggtggacaagaaagttgagcccaaatcttgtgacaaaactcacacatgcccaccgtgcccagcacct |
| | | gaactcctggggggaccgtcagtcttcctcttccccccaaaacccaaggacaccctcatgatctcccggacc |
| | | cctgaggtcacatgcgtggtggtggacgtgagccacgaagaccctgaggtcaagttcaactggtacgtggac |
| | | ggcgtggaggtgcataatgccaagacaaagccgcgggaggagcagtacaacagcacgtaccgggtggtcagc |
| | | gtcctcaccgtcctgcaccaggactggctgaatggcaaggagtacaagtgcaaggtctccaacaaagccctc |
| | | ccagcccccatcgagaaaaccatctccaaagccaaagggcagccccgagaaccacaggtgtacaccctgccc |
| | | ccatcccgggatgagctgaccaagaaccaggtcagcctgacctgcctggtcaaaggcttctatcccagcgac |
| | | atcgccgtggagtgggagagcaatgggcagccggagaacaactacaagaccacgcctcccgtgctggactcc |
| | | gacggctccttcttcctctacagcaagctcaccgtggacaagagcaggtggcagcaggggaacgtcttctca |
| | | tgctccgtgatgcatgaggctctgcacaaccactacacgcagaagagcctctccctgtctccgggtaaa |
| | | SEQ ID NO: 121 |
| | | ------------------------------------------------------------------------ |
| | | -------------------------------------- |
| | | tcctgggccctgactcaacccgcctccgtgtctgcgtctcctgggcagtcggtcaccatgtcctgcactgga |
| | | ttcggaaattataaccctgactcctggtaccaacaatacccaggcaaagcccccaaactcatcatttatgaa |
| | | gacaataaaagaccctcgggggtctctgatcgcttctctgcctccagacttggcagcacgtcttccctgaca |
| | | atctctaacgtccaggctgcggacgacgcccattatgtctgcgcctcctttgaatttttcggcggagggacc |
| | | aagctgaccgtcctgagtcagcccaaggctgccccctcggtcactctgttcccaccctcctctgaggagctt |
| | | caagccaacaaggccacactggtgtgtctcgtaagtgacttctacccgggagccgtgacagtggcctggaag |
| | | gcagatggcagccccgtcaaggtgggagtggagaccaccaaaccctccaaacaaagcaacaacaagtatgcg |
| | | gccagcagctacctgagcctgacgcccgagcagtggaagtcccacagaagctacagctgccgggtcacgcat |
| | | gaagggagcaccgtggagaagacagtggcccctgcagaatgctct |
| | | SEQ ID NO: 122 |
| Sequence 24 | N49P53 | cgtgtgacattacaacaatctggggctacagtgaagcagcctggggcctcagtgaccgtctcctgcgagact |
| | | tctggatacactttcaccaagtataccattcactgggtgcgacaggcccctggacagggtcttcagtgggtg |
| | | ggcagaatacacccccgaaccggtgccgtgaagtatgcaccgatatttcagggtaaagtgtccatgagtcga |
| | | gacttgtcacgcgacacagcctacctcggattgaccagactgacgctcgccgacacggctctatttttctgt |
| | | gcgaggggggcctttgaggcagatttaagtgggccaacttacccctttcaccactggggccaaggaacccta |
| | | gtcatcgtctccgcggcctccaccaagggcccatcggtcttccccctggcaccctcctccaagagcacctct |
| | | gggggcacagcggccctgggctgcctggtcaaggactacttccccgaaccggtgacggtgtcgtggaactca |
| | | ggcgccctgaccagcggcgtgcacaccttcccggctgtcctacagtcctcaggactctactccctcagcagc |
| | | gtggtgaccgtgccctccagcagcttgggcacccagacctacatctgcaacgtgaatcacaagcccagcaac |
| | | accaaggtggacaagaaagttgagcccaaatcttgtgacaaaactcacacatgcccaccgtgcccagcacct |
| | | gaactcctggggggaccgtcagtcttcctcttccccccaaaacccaaggacaccctcatgatctcccggacc |
| | | cctgaggtcacatgcgtggtggtggacgtgagccacgaagaccctgaggtcaagttcaactggtacgtggac |
| | | ggcgtggaggtgcataatgccaagacaaagccgcgggaggagcagtacaacagcacgtaccgggtggtcagc |
| | | gtcctcaccgtcctgcaccaggactggctgaatggcaaggagtacaagtgcaaggtctccaacaaagccctc |
| | | ccagcccccatcgagaaaaccatctccaaagccaaagggcagccccgagaaccacaggtgtacaccctgccc |
| | | ccatcccgggatgagctgaccaagaaccaggtcagcctgacctgcctggtcaaaggcttctatcccagcgac |
| | | atcgccgtggagtgggagagcaatgggcagccggagaacaactacaagaccacgcctcccgtgctggactcc |
| | | gacggctccttcttcctctacagcaagctcaccgtggacaagagcaggtggcagcaggggaacgtcttctca |
| | | tgctccgtgatgcatgaggctctgcacaaccactacacgcagaagagcctctccctgtctccgggtaaa |
| | | SEQ ID NO: 123 |
| | | ------------------------------------------------------------------------ |
| | | -------------------------------------- |
| | | tcctgggccctgactcaacccgcctccgtgtctgcgtctcctgggcagtcggtcaccatgtcctgcactgga |
| | | ttcggaaattataaccctgactcctggtaccaacaatacccaggcaaagcccccaaactcatcatttatgag |
| | | gacaataaaagaccctcgggagtctctaatcgcttctctgcctccagacttggcagcacgtcttccctgaca |
| | | atctctaacgtccaggccgctgacgacgcccattatgtctgcgcctcctttgaatttttcggcggagggacc |
| | | aagctgatcgtcctgagtcagcccaaggctgccccctcggtcactctgttcccaccctcctctgaggagctt |
| | | caagccaacaaggccacactggtgtgtctcgtaagtgacttctacccgggagccgtgacagtggcctggaag |
| | | gcagatggcagccccgtcaaggtgggagtggagaccaccaaaccctccaaacaaagcaacaacaagtatgcg |
| | | gccagcagctacctgagcctgacgcccgagcagtggaagtcccacagaagctacagctgccgggtcacgcat |
| | | gaagggagcaccgtggagaagacagtggcccctgcagaatgctct |
| | | SEQ ID NO: 124 |
| Sequence 25 | N49P54 | aacgtgcagttgatgcagtctgggactgaggtgaagaagtctggggcctcggtgacaatctcttgtgagacc |
| | | gctggattcaacttcatcgactccgtcatacactggctgcgccaggcccctggaggaggatttcagtggatg |
| | | gggtggatcaagcctcttagaggtgccgtcaattatccacagtttttgcagggcagggtctccatgacccgg |
| | | gacttgtccaccgacacggtgtacatggtcttgaatggactgacacctgacgacacaggcctttattactgc |
| | | gcgaaaggggcctttagagggggttctccctttggcttctggggccagggaactctgctcaccgtctcccca |
| | | gcctccaccaagggcccatcggtcttccccctggcaccctcctccaagagcacctctgggggcacagcggcc |
| | | ctgggctgcctggtcaaggactacttccccgaaccggtgacggtgtcgtggaactcaggcgccctgaccagc |
| | | ggcgtgcacaccttcccggctgtcctacagtcctcaggactctactccctcagcagcgtggtgaccgtgccc |
| | | tccagcagcttgggcacccagacctacatctgcaacgtgaatcacaagcccagcaacaccaaggtggacaag |
| | | aaagttgagcccaaatcttgtgacaaaactcacacatgcccaccgtgcccagcacctgaactcctgggggga |
| | | ccgtcagtcttcctcttccccccaaaacccaaggacaccctcatgatctcccggacccctgaggtcacatgc |
| | | gtggtggtggacgtgagccacgaagaccctgaggtcaagttcaactggtacgtggacggcgtggaggtgcat |
| | | aatgccaagacaaagccgcgggaggagcagtacaacagcacgtaccgggtggtcagcgtcctcaccgtcctg |
| | | caccaggactggctgaatggcaaggagtacaagtgcaaggtctccaacaaagccctcccagcccccatcgag |
| | | aaaaccatctccaaagccaaagggcagccccgagaaccacaggtgtacaccctgcccccatcccgggatgag |
| | | ctgaccaagaaccaggtcagcctgacctgcctggtcaaaggcttctatcccagcgacatcgccgtggagtgg |
| | | gagagcaatgggcagccggagaacaactacaagaccacgcctcccgtgctggactccgacggctccttcttc |
| | | ctctacagcaagctcaccgtggacaagagcaggtggcagcaggggaacgtcttctcatgctccgtgatgcat |
| | | gaggctctgcacaaccactacacgcagaagagcctctccctgtctccgggtaaa |
| | | SEQ ID NO: 125 |
| | | ------------------------------------------------------------------------ |
| | | -------------------------------------- |
| | | cagtctgccctgtctcagcctgtctccgtgtctgggtctcctggagagtcgatcaccatttcctgtactgga |
| | | gccaccacctggtatcaacaactcccaggcagaccccccaaactcatcatttatgacgtcactaaccggccc |
| | | tcaggcatttctagtcgtttctctggctccacgtctggccacacggcctccctgacaatctccggtctccag |
| | | gttgacgacgagggtctgtatcactgcgcctcacgtgaatttttcggcggagggaccaagctgaccgtcctg |
| | | agtcagcccaaggctgccccctcggtcactctgttcccaccctcctctgaggagcttcaagccaacaaggcc |
| | | acactggtgtgtctcgtaagtgacttctacccgggagccgtgacagtggcctggaaggcagatggcagcccc |
| | | gtcaaggtgggagtggagaccaccaaaccctccaaacaaagcaacaacaagtatgcggccagcagctacctg |
| | | agcctgacgcccgagcagtggaagtcccacagaagctacagctgccgggtcacgcatgaagggagcaccgtg |
| | | gagaagacagtggcccctgcagaatgctct |
| | | SEQ ID NO: 126 |
| Sequence 26 | N49P60 | caggtgcgactggtgcagtctgggcctcaggtgaagaagactggggcctcagtgagggtctcctgcgaaacc |
| | | tctggatacacgttcacctcctacttcatccattggttacgactgggccccggagaggggcttcagtggatg |
| | | gggtggatcaaccctttacatggtgccgtgaattatgaaaacaaatttaggggcagggtcacaatcaccagg |
| | | gacacgtccacagacacagtgtatttggacatgagcagactgacccctgacgacacggccgtctatttctgc |
| | | acaagaggaatcgttgctgatgggtggccctatggccactggggccagggaacccaagtcaccgtctccccg |
| | | gcctccaccaagggcccatcggtcttccccctggcaccctcctccaagagcacctctgggggcacagcggcc |
| | | ctgggctgcctggtcaaggactacttccccgaaccggtgacggtgtcgtggaactcaggcgccctgaccagc |
| | | ggcgtgcacaccttcccggctgtcctacagtcctcaggactctactccctcagcagcgtggtgaccgtgccc |
| | | tccagcagcttgggcacccagacctacatctgcaacgtgaatcacaagcccagcaacaccaaggtggacaag |
| | | aaagttgagcccaaatcttgtgacaaaactcacacatgcccaccgtgcccagcacctgaactcctgggggga |
| | | ccgtcagtcttcctcttccccccaaaacccaaggacaccctcatgatctcccggacccctgaggtcacatgc |
| | | gtggtggtggacgtgagccacgaagaccctgaggtcaagttcaactggtacgtggacggcgtggaggtgcat |
| | | aatgccaagacaaagccgcgggaggagcagtacaacagcacgtaccgggtggtcagcgtcctcaccgtcctg |
| | | caccaggactggctgaatggcaaggagtacaagtgcaaggtctccaacaaagccctcccagcccccatcgag |
| | | aaaaccatctccaaagccaaagggcagccccgagaaccacaggtgtacaccctgcccccatcccgggatgag |
| | | ctgaccaagaaccaggtcagcctgacctgcctggtcaaaggcttctatcccagcgacatcgccgtggagtgg |
| | | gagagcaatgggcagccggagaacaactacaagaccacgcctcccgtgctggactccgacggctccttcttc |
| | | ctctacagcaagctcaccgtggacaagagcaggtggcagcaggggaacgtcttctcatgctccgtgatgcat |
| | | gaggctctgcacaaccactacacgcagaagagcctctccctgtctccgggtaaa |
| | | SEQ ID NO: 127 |
| | | ------------------------------------------------------------------------ |
| | | -------------------------------------- |
| | | tcctgggccctgactcagcctgcctccgtgtctgggtctcctggacagtcggtcgccatctcctgcgctggc |
| | | ggcagcgtctcctggtaccaggtgctcccaggcagagcccccaaactcatcatttatgagggcgctaagcga |
| | | ccctcaggggtttctgctcgcttctctggctcccagtctggcaacacggcttacctgacaatttctgacctc |
| | | cagactgaggacgagggcatctacttctgctcttcacttcaattcttcggcggagggaccaaactgaccgtc |
| | | ctaagtcagcccaaggctgccccctcggtcactctgttcccaccctcctctgaggagcttcaagccaacaag |
| | | gccacactggtgtgtctcgtaagtgacttctacccgggagccgtgacagtggcctggaaggcagatggcagc |
| | | cccgtcaaggtgggagtggagaccaccaaaccctccaaacaaagcaacaacaagtatgcggccagcagctac |
| | | ctgagcctgacgcccgagcagtggaagtcccacagaagctacagctgccgggtcacgcatgaagggagcacc |
| | | gtggagaagacagtggcccctgcagaatgctct |
| | | SEQ ID NO: 128 |
| Sequence 27 | N49P61 | caggtgcgacttcagcagtctggtgtcgtggtgaggaagcctggggcctcagtgagaatttcctgcgagact |
| | | tctggattcaccttcatcgaccacattgtccattgggtgcggcgggcccctggacgaggctttgaatggatg |
| | | ggttggatcaagcctcttaggggtgccgtagattatgcaccccaacttcggggcaggatctccctgacgagg |
| | | gacatttacagtgaaaccgtctttatagacgtgagtcgactgacgtctggcgacacggcgatatacttttgt |
| | | tgtaaggccgccgcccctgaagaagcattcccccttcaatactggggccaggggacccaacttatcgtctcc |
| | | tcggcctccaccaagggcccatcggtcttccccctggcaccctcctccaagagcacctctgggggcacagcg |
| | | gccctgggctgcctggtcaaggactacttccccgaaccggtgacggtgtcgtggaactcaggcgccctgacc |
| | | agcggcgtgcacaccttcccggctgtcctacagtcctcaggactctactccctcagcagcgtggtgaccgtg |
| | | ccctccagcagcttgggcacccagacctacatctgcaacgtgaatcacaagcccagcaacaccaaggtggac |
| | | aagaaagttgagcccaaatcttgtgacaaaactcacacatgcccaccgtgcccagcacctgaactcctgggg |
| | | ggaccgtcagtcttcctcttccccccaaaacccaaggacaccctcatgatctcccggacccctgaggtcaca |
| | | tgcgtggtggtggacgtgagccacgaagaccctgaggtcaagttcaactggtacgtggacggcgtggaggtg |
| | | cataatgccaagacaaagccgcgggaggagcagtacaacagcacgtaccgggtggtcagcgtcctcaccgtc |
| | | ctgcaccaggactggctgaatggcaaggagtacaagtgcaaggtctccaacaaagccctcccagcccccatc |
| | | gagaaaaccatctccaaagccaaagggcagccccgagaaccacaggtgtacaccctgcccccatcccgggat |
| | | gagctgaccaagaaccaggtcagcctgacctgcctggtcaaaggcttctatcccagcgacatcgccgtggag |
| | | tgggagagcaatgggcagccggagaacaactacaagaccacgcctcccgtgctggactccgacggctccttc |
| | | ttcctctacagcaagctcaccgtggacaagagcaggtggcagcaggggaacgtcttctcatgctccgtgatg |
| | | catgaggctctgcacaaccactacacgcagaagagcctctccctgtctccgggtaaa |
| | | SEQ ID NO: 129 |
| | | ------------------------------------------------------------------------ |
| | | -------------------------------------- |
| | | caggctgccctgactcagcccgcctccgtgtccggctctcctggacagtcggtcaccatttcctgcctttat |
| | | gccaatgtagatatctgctggtatcaactacacccgggcagagcccccaaacttctaattgttgacaataat |
| | | aagcggccctcaggagtctctcctcgcttctctggctccaagtctggcaccacggcctccctgacaatctct |
| | | ggacttcaggctgacgacgaggctgaatatcactgctcttcaagaacattttttggcggggggaccaagttg |
| | | accgtcctgagtcagcccaaggctgccccctcggtcactctgttcccaccctcctctgaggagcttcaagcc |
| | | aacaaggccacactggtgtgtctcgtaagtgacttctacccgggagccgtgacagtggcctggaaggcagat |
| | | ggcagccccgtcaaggtgggagtggagaccaccaaaccctccaaacaaagcaacaacaagtatgcggccagc |
| | | agctacctgagcctgacgcccgagcagtggaagtcccacagaagctacagctgccgggtcacgcatgaaggg |
| | | agcaccgtggagaagacagtggcccctgcagaatgctct |
| | | SEQ ID NO: 130 |
| Sequence 28 | N49P62 | caggtgcgacttcagcagtctggtgtcgtggtgaggaagcctggggcctcagtgagactttcctgcgagacg |
| | | tctggattcaaattcatcgaccacattgtcaactgggtgcggcgggcccctggacgaggctttgaatggatg |
| | | ggttggatcaagcctcttgggggtgtcgctgattatgcaccccaacatcggggcaggatctcactgacgagg |
| | | gacatttacactgaaaccgtctttatagacctgagtcgactgacgtctggcgacacggcgatttatttctgt |
| | | tgtaaggccgccgcccctgatgaagcattcccccttgaatactggggccaggggacccaacttatcgtctcc |
| | | ccggcctccaccaagggcccatcggtcttccccctggcaccctcctccaagagcacctctgggggcacagcg |
| | | gccctgggctgcctggtcaaggactacttccccgaaccggtgacggtgtcgtggaactcaggcgccctgacc |
| | | agcggcgtgcacaccttcccggctgtcctacagtcctcaggactctactccctcagcagcgtggtgaccgtg |
| | | ccctccagcagcttgggcacccagacctacatctgcaacgtgaatcacaagcccagcaacaccaaggtggac |
| | | aagaaagttgagcccaaatcttgtgacaaaactcacacatgcccaccgtgcccagcacctgaactcctgggg |
| | | ggaccgtcagtcttcctcttccccccaaaacccaaggacaccctcatgatctcccggacccctgaggtcaca |
| | | tgcgtggtggtggacgtgagccacgaagaccctgaggtcaagttcaactggtacgtggacggcgtggaggtg |
| | | cataatgccaagacaaagccgcgggaggagcagtacaacagcacgtaccgggtggtcagcgtcctcaccgtc |
| | | ctgcaccaggactggctgaatggcaaggagtacaagtgcaaggtctccaacaaagccctcccagcccccatc |
| | | gagaaaaccatctccaaagccaaagggcagccccgagaaccacaggtgtacaccctgcccccatcccgggat |
| | | gagctgaccaagaaccaggtcagcctgacctgcctggtcaaaggcttctatcccagcgacatcgccgtggag |
| | | tgggagagcaatgggcagccggagaacaactacaagaccacgcctcccgtgctggactccgacggctccttc |
| | | ttcctctacagcaagctcaccgtggacaagagcaggtggcagcaggggaacgtcttctcatgctccgtgatg |
| | | catgaggctctgcacaaccactacacgcagaagagcctctccctgtctccgggtaaa |
| | | SEQ ID NO: 131 |
| | | ------------------------------------------------------------------------ |
| | | -------------------------------------- |
| | | caggctgccctgactcagcccgcctccgtgtccggctctcctggacagtcggtcaccatttcctgcctttat |
| | | gccaatgtagatatctgctggtatcaaatacagccgggcagattacccaaacttctgattgttgacaataat |
| | | aggcgaccctcaggagtctctcctcgcttctctggctccaagtctggcaccacggcctccctgacaatctct |
| | | ggacttcaggctgacgacgaggctgaatatcactgctcttcaacaacattttttggcggggggaccaagttg |
| | | accgtcctcagtcagcccaaggctgccccctcggtcactctgttcccaccctcctctgaggagcttcaagcc |
| | | aacaaggccacactggtgtgtctcgtaagtgacttctacccgggagccgtgacagtggcctggaaggcagat |
| | | ggcagccccgtcaaggtgggagtggagaccaccaaaccctccaaacaaagcaacaacaagtatgcggccagc |
| | | agctacctgagcctgacgcccgagcagtggaagtcccacagaagctacagctgccgggtcacgcatgaaggg |
| | | agcaccgtggagaagacagtggcccctgcagaatgctct |
| | | SEQ ID NO: 132 |
| Sequence 29 | N49P63 | caggtgcgacttgtgcagtctggtcccgtgatgagaaagcctggggcctcagtgagaatttcttgcgagaca |
| | | tctggattcgccttcttggaccacattgtccactgggtgcggcgggcccctggacgcggctttgaatggatg |
| | | ggttgggttaagaccattgggggtgtcgttgattatgcaccccaccttaggggcaggatctccgtgacgaga |
| | | gacgtctttagtgaaaccgtctttctggacttgagtcgactgacgtctggcgacacggcgatgtatttttgt |
| | | tctaaggccgccgcccctgacgaagccttcccccttgaattttggggccaggggacccaagtcatcgtctcc |
| | | tcggcctccaccaagggcccatcggtcttccccctggcaccctcctccaagagcacctctgggggcacagcg |
| | | gccctgggctgcctggtcaaggactacttccccgaaccggtgacggtgtcgtggaactcaggcgccctgacc |
| | | agcggcgtgcacaccttcccggctgtcctacagtcctcaggactctactccctcagcagcgtggtgaccgtg |
| | | ccctccagcagcttgggcacccagacctacatctgcaacgtgaatcacaagcccagcaacaccaaggtggac |
| | | aagaaagttgagcccaaatcttgtgacaaaactcacacatgcccaccgtgcccagcacctgaactcctgggg |
| | | ggaccgtcagtcttcctcttccccccaaaacccaaggacaccctcatgatctcccggacccctgaggtcaca |
| | | tgcgtggtggtggacgtgagccacgaagaccctgaggtcaagttcaactggtacgtggacggcgtggaggtg |
| | | cataatgccaagacaaagccgcgggaggagcagtacaacagcacgtaccgggtggtcagcgtcctcaccgtc |
| | | ctgcaccaggactggctgaatggcaaggagtacaagtgcaaggtctccaacaaagccctcccagcccccatc |
| | | gagaaaaccatctccaaagccaaagggcagccccgagaaccacaggtgtacaccctgcccccatcccgggat |
| | | gagctgaccaagaaccaggtcagcctgacctgcctggtcaaaggcttctatcccagcgacatcgccgtggag |
| | | tgggagagcaatgggcagccggagaacaactacaagaccacgcctcccgtgctggactccgacggctccttc |
| | | ttcctctacagcaagctcaccgtggacaagagcaggtggcagcaggggaacgtcttctcatgctccgtgatg |
| | | catgaggctctgcacaaccactacacgcagaagagcctctccctgtctccgggtaaa |
| | | SEQ ID NO: 133 |
| | | ------------------------------------------------------------------------ |
| | | -------------------------------------- |
| | | caggctgccctgactcagcccgcctccgtgtccggctctcctggacagtcggtcaccatttcttgcctttat |
| | | gccaatgtggatatctgctggtatcaacttcacccgggcagagcccccaaacttcttattcttgacaataat |
| | | aaacggccctcaggagtctctagtcgcttctccggttccaagtctggcaccacggcctccctaaccatctct |
| | | gaccttcaggctgacgacgaggctgaatatcactgctcttcaacaacattttttggcggggggaccaggttg |
| | | accgtcctgagtcagcccaaggctgccccctcggtcactctgttcccaccctcctctgaggagcttcaagcc |
| | | aacaaggccacactggtgtgtctcgtaagtgacttctacccgggagccgtgacagtggcctggaaggcagat |
| | | ggcagccccgtcaaggtgggagtggagaccaccaaaccctccaaacaaagcaacaacaagtatgcggccagc |
| | | agctacctgagcctgacgcccgagcagtggaagtcccacagaagctacagctgccgggtcacgcatgaaggg |
| | | agcaccgtggagaagacagtggcccctgcagaatgctct |
| | | SEQ ID NO: 134 |
| Sequence 30 | N49P64 | caggtgcgacttgtgcagtctggtcccgtggtgagaaagcctgggacctcagtgagaatttcttgcgagaca |
| | | tctggattcgccttcttggaccacattgtccactgggtgcggcgggcccctggacgcggctttgaatggatg |
| | | ggttgggttaagaccattgggggtgtcgttgattatgcaccccaccttaggggcaggatctccgtgacgagg |
| | | gacgtatttagtgaaatcgtctttatggagttgagtcgactgacgtctggcgacacggcgatgtatttttgt |
| | | tctaaggccgccgcccctgacgaagccttcccccttgaattttggggccaggggacccaagtcatcgtctcc |
| | | tcggcctccaccaagggcccatcggtcttccccctggcaccctcctccaagagcacctctgggggcacagcg |
| | | gccctgggctgcctggtcaaggactacttccccgaaccggtgacggtgtcgtggaactcaggcgccctgacc |
| | | agcggcgtgcacaccttcccggctgtcctacagtcctcaggactctactccctcagcagcgtggtgaccgtg |
| | | ccctccagcagcttgggcacccagacctacatctgcaacgtgaatcacaagcccagcaacaccaaggtggac |
| | | aagaaagttgagcccaaatcttgtgacaaaactcacacatgcccaccgtgcccagcacctgaactcctgggg |
| | | ggaccgtcagtcttcctcttccccccaaaacccaaggacaccctcatgatctcccggacccctgaggtcaca |
| | | tgcgtggtggtggacgtgagccacgaagaccctgaggtcaagttcaactggtacgtggacggcgtggaggtg |
| | | cataatgccaagacaaagccgcgggaggagcagtacaacagcacgtaccgggtggtcagcgtcctcaccgtc |
| | | ctgcaccaggactggctgaatggcaaggagtacaagtgcaaggtctccaacaaagccctcccagcccccatc |
| | | gagaaaaccatctccaaagccaaagggcagccccgagaaccacaggtgtacaccctgcccccatcccgggat |
| | | gagctgaccaagaaccaggtcagcctgacctgcctggtcaaaggcttctatcccagcgacatcgccgtggag |
| | | tgggagagcaatgggcagccggagaacaactacaagaccacgcctcccgtgctggactccgacggctccttc |
| | | ttcctctacagcaagctcaccgtggacaagagcaggtggcagcaggggaacgtcttctcatgctccgtgatg |
| | | catgaggctctgcacaaccactacacgcagaagagcctctccctgtctccgggtaaa |
| | | SEQ ID NO: 135 |
| | | ------------------------------------------------------------------------ |
| | | -------------------------------------- |
| | | caggctgccctgactcagcccgcctccgtgtccggctctcctggacagtcggtcaccatttcctgcctttat |
| | | gccaatgtggatatctgctggtatcaacttcacccgggcagagcccccaaacttctaattgttgacaataat |
| | | aagcggccctcaggagtctctagtcgcttctctggttccaagtctggcaccacggcctccctaacaatctct |
| | | gatcttcaggctgacgacgaggctgaatatcactgctcttcaacaacattttttggcggggggaccaggttg |
| | | accgtcctgagtcagcccaaggctgccccctcggtcactctgttcccaccctcctctgaggagcttcaagcc |
| | | aacaaggccacactggtgtgtctcgtaagtgacttctacccgggagccgtgacagtggcctggaaggcagat |
| | | ggcagccccgtcaaggtgggagtggagaccaccaaaccctccaaacaaagcaacaacaagtatgcggccagc |
| | | agctacctgagcctgacgcccgagcagtggaagtcccacagaagctacagctgccgggtcacgcatgaaggg |
| | | agcaccgtggagaagacagtggcccctgcagaatgctct |
| | | SEQ ID NO: 136 |
| Sequence 31 | N49P65 | caagtgcaactggtgcagtctggggctggggtgaagaagcctggggcctcagtgagggtctcctgcgagaca |
| | | tccggattcaagttcaccgagtactttatccactttttacgacaggcccctggacaagggcttgagtggatg |
| | | ggatggctcaaccctctcagaggtgccgtcaactatccacggaagtttcagggcagagtcactttgaccagg |
| | | gacatctacaccaccaccgtctacatgcaacttaacggtctgacccctgacgacacggccgtctactactgt |
| | | gccagagcggtctttaatgaagctttcccctttgactactggggccagggaagcctggtcaccgtctcctca |
| | | gcctccaccaagggcccatcggtcttccccctggcaccctcctccaagagcacctctgggggcacagcggcc |
| | | ctgggctgcctggtcaaggactacttccccgaaccggtgacggtgtcgtggaactcaggcgccctgaccagc |
| | | ggcgtgcacaccttcccggctgtcctacagtcctcaggactctactccctcagcagcgtggtgaccgtgccc |
| | | tccagcagcttgggcacccagacctacatctgcaacgtgaatcacaagcccagcaacaccaaggtggacaag |
| | | aaagttgagcccaaatcttgtgacaaaactcacacatgcccaccgtgcccagcacctgaactcctgggggga |
| | | ccgtcagtcttcctcttccccccaaaacccaaggacaccctcatgatctcccggacccctgaggtcacatgc |
| | | gtggtggtggacgtgagccacgaagaccctgaggtcaagttcaactggtacgtggacggcgtggaggtgcat |
| | | aatgccaagacaaagccgcgggaggagcagtacaacagcacgtaccgggtggtcagcgtcctcaccgtcctg |
| | | caccaggactggctgaatggcaaggagtacaagtgcaaggtctccaacaaagccctcccagcccccatcgag |
| | | aaaaccatctccaaagccaaagggcagccccgagaaccacaggtgtacaccctgcccccatcccgggatgag |
| | | ctgaccaagaaccaggtcagcctgacctgcctggtcaaaggcttctatcccagcgacatcgccgtggagtgg |
| | | gagagcaatgggcagccggagaacaactacaagaccacgcctcccgtgctggactccgacggctccttcttc |
| | | ctctacagcaagctcaccgtggacaagagcaggtggcagcaggggaacgtcttctcatgctccgtgatgcat |
| | | gaggctctgcacaaccactacacgcagaagagcctctccctgtctccgggtaaa |
| | | SEQ ID NO: 137 |
| | | ------------------------------------------------------------------------ |
| | | -------------------------------------- |
| | | tcctgggcccagactcagcctgcctccgtgtctgggtctcctggacagtcgatcaccatctcctgcgctgga |
| | | atcgtcagtgatgcctggtaccagcaatacccaggcagaccccccagactcatcctttatgacggcgataag |
| | | cggccctcaggggtttctcctcgtttttctgcctccagggccggcaagacggcctccctgacaatttctggg |
| | | ctgcaggctgacgacgaggcttattatcactgcgcgtcaagggaattttttggaggcgtgaccaagttgacc |
| | | gtcctaagtcagcccaaggctgccccctcggtcactctgttcccaccctcctctgaggagcttcaagccaac |
| | | aaggccacactggtgtgtctcgtaagtgacttctacccgggagccgtgacagtggcctggaaggcagatggc |
| | | agccccgtcaaggtgggagtggagaccaccaaaccctccaaacaaagcaacaacaagtatgcggccagcagc |
| | | tacctgagcctgacgcccgagcagtggaagtcccacagaagctacagctgccgggtcacgcatgaagggagc |
| | | accgtggagaagacagtggcccctgcagaatgctct |
| | | SEQ ID NO: 138 |
| Sequence 32 | N49P66 | caggtgcgacttcagcagtctggtgtcgtggtgaggaagcctggggcctcagtgagactttcctgcgagacg |
| | | tctggcttcaaattcatcgaccacattgtcaactgggtgcggcgggcccctggacgaggctttgaatggatg |
| | | ggttggatcaagcctcttgggggtgtcgctgattatgcaccccaacatcggggcaggatctcactgacgagg |
| | | gacatttacactgaaaccgtctttatagacctgagtcgactgacgtctggcgacacggcgatttatttttgt |
| | | tgtaaggccgccgcccctgatgaagcattcccccttgaatactggggccaggggacccaacttatcgtctcc |
| | | ccggcctccaccaagggcccatcggtcttccccctggcaccctcctccaagagcacctctgggggcacagcg |
| | | gccctgggctgcctggtcaaggactacttccccgaaccggtgacggtgtcgtggaactcaggcgccctgacc |
| | | agcggcgtgcacaccttcccggctgtcctacagtcctcaggactctactccctcagcagcgtggtgaccgtg |
| | | ccctccagcagcttgggcacccagacctacatctgcaacgtgaatcacaagcccagcaacaccaaggtggac |
| | | aagaaagttgagcccaaatcttgtgacaaaactcacacatgcccaccgtgcccagcacctgaactcctgggg |
| | | ggaccgtcagtcttcctcttccccccaaaacccaaggacaccctcatgatctcccggacccctgaggtcaca |
| | | tgcgtggtggtggacgtgagccacgaagaccctgaggtcaagttcaactggtacgtggacggcgtggaggtg |
| | | cataatgccaagacaaagccgcgggaggagcagtacaacagcacgtaccgggtggtcagcgtcctcaccgtc |
| | | ctgcaccaggactggctgaatggcaaggagtacaagtgcaaggtctccaacaaagccctcccagcccccatc |
| | | gagaaaaccatctccaaagccaaagggcagccccgagaaccacaggtgtacaccctgcccccatcccgggat |
| | | gagctgaccaagaaccaggtcagcctgacctgcctggtcaaaggcttctatcccagcgacatcgccgtggag |
| | | tgggagagcaatgggcagccggagaacaactacaagaccacgcctcccgtgctggactccgacggctccttc |
| | | ttcctctacagcaagctcaccgtggacaagagcaggtggcagcaggggaacgtcttctcatgctccgtgatg |
| | | catgaggctctgcacaaccactacacgcagaagagcctctccctgtctccgggtaaa |
| | | SEQ ID NO: 139 |
| | | ------------------------------------------------------------------------ |
| | | -------------------------------------- |
| | | caggctgccctgactcagcccgcctccgtgtccggctctcctggacagtcggtcaccatttcctgcctttat |
| | | gccaatgtagatatctgctggtatcaaatacagccgggcagattacccaaacttctgattgttgacaatgat |
| | | aggcgaccctcaggagtctctcctcgcttctctggctccaagtctggcaccacggcctccctgacaatctct |
| | | ggacttcaggctgacgacgaggctgaatatcactgctcttcaacaacattttttggcggggggaccaagttg |
| | | accgtcctcagtcagcccaaggctgccccctcggtcactctgttcccaccctcctctgaggagcttcaagcc |
| | | aacaaggccacactggtgtgtctcgtaagtgacttctacccgggagccgtgacagtggcctggaaggcagat |
| | | ggcagccccgtcaaggtgggagtggagaccaccaaaccctccaaacaaagcaacaacaagtatgcggccagc |
| | | agctacctgagcctgacgcccgagcagtggaagtcccacagaagctacagctgccgggtcacgcatgaaggg |
| | | agcaccgtggagaagacagtggcccctgcagaatgctct |
| | | SEQ ID NO: 140 |
| Sequence 33 | N49P67 | caggtgcgacttgtgcagtctggtcccgtgatgagaaagcctggggcctcagtgagaatttcttgcgagaca |
| | | tctggattcgccttcttggaccacattgtccactgggtgcggcgggcccctggacgcggctttgaatggatg |
| | | ggttgggttaagaccattgggggtgtcgttgattatgcaccccaccttaggggcaggatctccgtgacgaga |
| | | gacgtctttagtgaaaccgtctttctggacttgagtcgactgacgtctggcgacacggcgatgtatttttgt |
| | | tctaaggccgccgcccctgacgaagccttcccccttgaattttggggccaggggacccaagtcatcgtctcc |
| | | tcggcctccaccaagggcccatcggtcttccccctggcaccctcctccaagagcacctctgggggcacagcg |
| | | gccctgggctgcctggtcaaggactacttccccgaaccggtgacggtgtcgtggaactcaggcgccctgacc |
| | | agcggcgtgcacaccttcccggctgtcctacagtcctcaggactctactccctcagcagcgtggtgaccgtg |
| | | ccctccagcagcttgggcacccagacctacatctgcaacgtgaatcacaagcccagcaacaccaaggtggac |
| | | aagaaagttgagcccaaatcttgtgacaaaactcacacatgcccaccgtgcccagcacctgaactcctgggg |
| | | ggaccgtcagtcttcctcttccccccaaaacccaaggacaccctcatgatctcccggacccctgaggtcaca |
| | | tgcgtggtggtggacgtgagccacgaagaccctgaggtcaagttcaactggtacgtggacggcgtggaggtg |
| | | cataatgccaagacaaagccgcgggaggagcagtacaacagcacgtaccgggtggtcagcgtcctcaccgtc |
| | | ctgcaccaggactggctgaatggcaaggagtacaagtgcaaggtctccaacaaagccctcccagcccccatc |
| | | gagaaaaccatctccaaagccaaagggcagccccgagaaccacaggtgtacaccctgcccccatcccgggat |
| | | gagctgaccaagaaccaggtcagcctgacctgcctggtcaaaggcttctatcccagcgacatcgccgtggag |
| | | tgggagagcaatgggcagccggagaacaactacaagaccacgcctcccgtgctggactccgacggctccttc |
| | | ttcctctacagcaagctcaccgtggacaagagcaggtggcagcaggggaacgtcttctcatgctccgtgatg |
| | | catgaggctctgcacaaccactacacgcagaagagcctctccctgtctccgggtaaa |
| | | SEQ ID NO: 141 |
| | | ------------------------------------------------------------------------ |
| | | -------------------------------------- |
| | | caggctgccctgactcagcccgcctccgtgtccggctctcctggacagtcggtcaccatttcttgcctttat |
| | | gccaatgtggatatctgctggtatcaacttcacccgggcagagcccccaaacttctaattcttgacaataat |
| | | aaacggccctcaggagtctctagtcgcttctccggttccaagtctggcaccacggcctccctaaccatctct |
| | | gaccttcaggctgacgacgaggctgaatatcactgctcttcaacaactttttttggcggggggaccaggttg |
| | | accgtcctgagtcagcccaaggctgccccctcggtcactctgttcccaccctcctctgaggagcttcaagcc |
| | | aacaaggccacactggtgtgtctcgtaagtgacttctacccgggagccgtgacagtggcctggaaggcagat |
| | | ggcagccccgtcaaggtgggagtggagaccaccaaaccctccaaacaaagcaacaacaagtatgcggccagc |
| | | agctacctgagcctgacgcccgagcagtggaagtcccacagaagctacagctgccgggtcacgcatgaaggg |
| | | agcaccgtggagaagacagtggcccctgcagaatgctct |
| | | SEQ ID NO: 142 |
| Sequence 34 | N49P68 | cacgtgcagttgaggcagtctgggactgaggcgaagaagtctggggcctcggtgacaatctcttgtgagacc |
| | | gctggattcaacttcatcgactccgtcatacactggctgcgccaggcccctggtgggggatttcagtggatg |
| | | gggtggatcaagcctcttagaggtggcgtcaattatccacattatttgcagggcagaatctccatgacccgg |
| | | gacttgtccagtgacacggtttacatggtcttaaatagactgacacctgccgacacaggcctttattactgc |
| | | gcgaaaggggcctttggggggagttctccctttggcttctggggccagggaactctgctcaccgtctcccca |
| | | gcctccaccaagggcccatcggtcttccccctggcaccctcctccaagagcacctctgggggcacagcggcc |
| | | ctgggctgcctggtcaaggactacttccccgaaccggtgacggtgtcgtggaactcaggcgccctgaccagc |
| | | ggcgtgcacaccttcccggctgtcctacagtcctcaggactctactccctcagcagcgtggtgaccgtgccc |
| | | tccagcagcttgggcacccagacctacatctgcaacgtgaatcacaagcccagcaacaccaaggtggacaag |
| | | aaagttgagcccaaatcttgtgacaaaactcacacatgcccaccgtgcccagcacctgaactcctgggggga |
| | | ccgtcagtcttcctcttccccccaaaacccaaggacaccctcatgatctcccggacccctgaggtcacatgc |
| | | gtggtggtggacgtgagccacgaagaccctgaggtcaagttcaactggtacgtggacggcgtggaggtgcat |
| | | aatgccaagacaaagccgcgggaggagcagtacaacagcacgtaccgggtggtcagcgtcctcaccgtcctg |
| | | caccaggactggctgaatggcaaggagtacaagtgcaaggtctccaacaaagccctcccagcccccatcgag |
| | | aaaaccatctccaaagccaaagggcagccccgagaaccacaggtgtacaccctgcccccatcccgggatgag |
| | | ctgaccaagaaccaggtcagcctgacctgcctggtcaaaggcttctatcccagcgacatcgccgtggagtgg |
| | | gagagcaatgggcagccggagaacaactacaagaccacgcctcccgtgctggactccgacggctccttcttc |
| | | ctctacagcaagctcaccgtggacaagagcaggtggcagcaggggaacgtcttctcatgctccgtgatgcat |
| | | gaggctctgcacaaccactacacgcagaagagcctctccctgtctccgggtaaa |
| | | SEQ ID NO: 143 |
| | | ------------------------------------------------------------------------ |
| | | -------------------------------------- |
| | | cagtctgccctgtctcagcctgtctccgtgtctgggtctcctggagagtcgatcaccatttcctgtactgaa |
| | | gccaccacctggtatcaacaactcccaggcaaaccccccaaactcatcatttatgacgtgaccaaccggccc |
| | | tcaggcatttcaagtcgtttctctggctccatgtctggtcgcacggcctccctgacaatctccggtctccag |
| | | gttgacgacgagggtctctatcactgtgcctcacgtgaatttttcggcggggggaccaagctgaccgtcctg |
| | | agtcagcccaaggctgccccctcggtcactctgttcccaccctcctctgaggagcttcaagccaacaaggcc |
| | | acactggtgtgtctcgtaagtgacttctacccgggagccgtgacagtggcctggaaggcagatggcagcccc |
| | | gtcaaggtgggagtggagaccaccaaaccctccaaacaaagcaacaacaagtatgcggccagcagctacctg |
| | | agcctgacgcccgagcagtggaagtcccacagaagctacagctgccgggtcacgcatgaagggagcaccgtg |
| | | gagaagacagtggcccctgcagaatgctct |
| | | SEQ ID NO: 144 |
| Sequence 35 | N49P69 | cacgtgcaattgatgcagtctgggactcaggcgaagaagtctggggcctcggtgacaatttcttgtgagacc |
| | | gctggattcaagttcatcgactccgtcatacactggctgcgccaggcccctggagggggatttcagtggatg |
| | | gggtggatcaagcctcttggaggtgccgtcaactatccaccctatttgcagggcaggatctccttgacccgt |
| | | gacttgtccaccgacacaatttacatggtcttgaatggactgacacctgccgacacaggcttttattactgc |
| | | gccaaaggggcctttggggggggttctccctttggcttctggggccaggggactctgctcaccgtctcccca |
| | | gcctccaccaagggcccatcggtcttccccctggcaccctcctccaagagcacctctgggggcacagcggcc |
| | | ctgggctgcctggtcaaggactacttccccgaaccggtgacggtgtcgtggaactcaggcgccctgaccagc |
| | | ggcgtgcacaccttcccggctgtcctacagtcctcaggactctactccctcagcagcgtggtgaccgtgccc |
| | | tccagcagcttgggcacccagacctacatctgcaacgtgaatcacaagcccagcaacaccaaggtggacaag |
| | | aaagttgagcccaaatcttgtgacaaaactcacacatgcccaccgtgcccagcacctgaactcctgggggga |
| | | ccgtcagtcttcctcttccccccaaaacccaaggacaccctcatgatctcccggacccctgaggtcacatgc |
| | | gtggtggtggacgtgagccacgaagaccctgaggtcaagttcaactggtacgtggacggcgtggaggtgcat |
| | | aatgccaagacaaagccgcgggaggagcagtacaacagcacgtaccgggtggtcagcgtcctcaccgtcctg |
| | | caccaggactggctgaatggcaaggagtacaagtgcaaggtctccaacaaagccctcccagcccccatcgag |
| | | aaaaccatctccaaagccaaagggcagccccgagaaccacaggtgtacaccctgcccccatcccgggatgag |
| | | ctgaccaagaaccaggtcagcctgacctgcctggtcaaaggcttctatcccagcgacatcgccgtggagtgg |
| | | gagagcaatgggcagccggagaacaactacaagaccacgcctcccgtgctggactccgacggctccttcttc |
| | | ctctacagcaagctcaccgtggacaagagcaggtggcagcaggggaacgtcttctcatgctccgtgatgcat |
| | | gaggctctgcacaaccactacacgcagaagagcctctccctgtctccgggtaaa |
| | | SEQ ID NO: 145 |
| | | ------------------------------------------------------------------------ |
| | | -------------------------------------- |
| | | cagtctgccctgtctcagcctgtctccgtgtctgggtctcctggagactcgatcaccatttcttgttttgga |
| | | gccaccacctggtatcaacaactcccaggcagaccccccaaactcatcatttatgacgtgactaaccggccc |
| | | tcaggcatttcaggtcgtttctctggctccatgtctggtcaaaaggcctccctgacaatctccggtctccag |
| | | gttgacgacgagggtctctatcactgcgcctcacgtgaatttttcggcggggggaccaaactgaccgtcctg |
| | | agtcagcccaaggctgccccctcggtcactctgttcccaccctcctctgaggagcttcaagccaacaaggcc |
| | | acactggtgtgtctcgtaagtgacttctacccgggagccgtgacagtggcctggaaggcagatggcagcccc |
| | | gtcaaggtgggagtggagaccaccaaaccctccaaacaaagcaacaacaagtatgcggccagcagctacctg |
| | | agcctgacgcccgagcagtggaagtcccacagaagctacagctgccgggtcacgcatgaagggagcaccgtg |
| | | gagaagacagtggcccctgcagaatgctct |
| | | SEQ ID NO: 146 |
| Sequence 36 | N49P70 | cacgtgcagttgaggcagtctgggactgaggcgaagaagtctggggcctcggtgacaatctcttgtgagacc |
| | | gctggattcaacttcatcgactccgtcatacactggctgcgccaggcccctggtgggggatttcagtggatg |
| | | gggtggatcaagcctcttagaggtggcgtcaattatccacattatttgcagggcagaatctccatgacccgg |
| | | gacttgtccagtgacacggtttacatggtcttaaatagactgacacctgacgacacaggcctttactactgc |
| | | gcgaaaggggcctttggggggagttctccctttggcttctggggccagggaactctgctcaccgtctcccca |
| | | gcctccaccaagggcccatcggtcttccccctggcaccctcctccaagagcacctctgggggcacagcggcc |
| | | ctgggctgcctggtcaaggactacttccccgaaccggtgacggtgtcgtggaactcaggcgccctgaccagc |
| | | ggcgtgcacaccttcccggctgtcctacagtcctcaggactctactccctcagcagcgtggtgaccgtgccc |
| | | tccagcagcttgggcacccagacctacatctgcaacgtgaatcacaagcccagcaacaccaaggtggacaag |
| | | aaagttgagcccaaatcttgtgacaaaactcacacatgcccaccgtgcccagcacctgaactcctgggggga |
| | | ccgtcagtcttcctcttccccccaaaacccaaggacaccctcatgatctcccggacccctgaggtcacatgc |
| | | gtggtggtggacgtgagccacgaagaccctgaggtcaagttcaactggtacgtggacggcgtggaggtgcat |
| | | aatgccaagacaaagccgcgggaggagcagtacaacagcacgtaccgggtggtcagcgtcctcaccgtcctg |
| | | caccaggactggctgaatggcaaggagtacaagtgcaaggtctccaacaaagccctcccagcccccatcgag |
| | | aaaaccatctccaaagccaaagggcagccccgagaaccacaggtgtacaccctgcccccatcccgggatgag |
| | | ctgaccaagaaccaggtcagcctgacctgcctggtcaaaggcttctatcccagcgacatcgccgtggagtgg |
| | | gagagcaatgggcagccggagaacaactacaagaccacgcctcccgtgctggactccgacggctccttcttc |
| | | ctctacagcaagctcaccgtggacaagagcaggtggcagcaggggaacgtcttctcatgctccgtgatgcat |
| | | gaggctctgcacaaccactacacgcagaagagcctctccctgtctccgggtaaa |
| | | SEQ ID NO: 147 |
| | | ------------------------------------------------------------------------ |
| | | -------------------------------------- |
| | | cagtctgccctgtctcagcctgtctccgtgtctgggtctcctggagagtcgatcaccatttcctgtactgaa |
| | | gccaccacctggtatcaacaactcccagggagatcccccaaactcattatttatgacgtgaccaaccggccc |
| | | tcaggcatttcaagtcgtttctctggctccatgtctggtcgcacggcctccctgacaatctccggtctccag |
| | | gttgacgacgagggtctctatcactgtgcctcacgtgaatttttcggcggggggaccaagctgaccgtcctc |
| | | agtcagcccaaggctgccccctcggtcactctgttcccaccctcctctgaggagcttcaagccaacaaggcc |
| | | acactggtgtgtctcgtaagtgacttctacccgggagccgtgacagtggcctggaaggcagatggcagcccc |
| | | gtcaaggtgggagtggagaccaccaaaccctccaaacaaagcaacaacaagtatgcggccagcagctacctg |
| | | agcctgacgcccgagcagtggaagtcccacagaagctacagctgccgggtcacgcatgaagggagcaccgtg |
| | | gagaagacagtggcccctgcagaatgctct |
| | | SEQ ID NO: 148 |
| Sequence 37 | N49P71 | cgtgttacattacaacagtctggggctacagtgaggcagcctggggcctcagtcaccgtctcctgcgagact |
| | | tctggattcaccttcatcaaatataccattcactgggtgcgacaggcccctggacagggtcttcagtggggg |
| | | gaagaatacacccccgaaccggtgccgtgaagtttgcaccgatatttcagggtaaattttccatgagtcgag |
| | | acttgtcacgcgacacagcctacctcggattgaccagactgacactcgccgacacggctctatttttctgtg |
| | | cgaggggggcctttgaggcagatttatatgggccaacttacccctttcaccactggggccaaggaacccaag |
| | | tcaccgtctccgcggcctccaccaagggcccatcggtcttccccctggcaccctcctccaagagcacctctg |
| | | ggggcacagcggccctgggctgcctggtcaaggactacttccccgaaccggtgacggtgtcgtggaactcag |
| | | gcgccctgaccagcggcgtgcacaccttcccggctgtcctacagtcctcaggactctactccctcagcagcg |
| | | tggtgaccgtgccctccagcagcttgggcacccagacctacatctgcaacgtgaatcacaagcccagcaaca |
| | | ccaaggtggacaagaaagttgagcccaaatcttgtgacaaaactcacacatgcccaccgtgcccagcacctg |
| | | aactcctggggggaccgtcagtcttcctcttccccccaaaacccaaggacaccctcatgatctcccggaccc |
| | | ctgaggtcacatgcgtggtggtggacgtgagccacgaagaccctgaggtcaagttcaactggtacgtggacg |
| | | gcgtggaggtgcataatgccaagacaaagccgcgggaggagcagtacaacagcacgtaccgggtggtcagcg |
| | | tcctcaccgtcctgcaccaggactggctgaatggcaaggagtacaagtgcaaggtctccaacaaagccctcc |
| | | cagcccccatcgagaaaaccatctccaaagccaaagggcagccccgagaaccacaggtgtacaccctgcccc |
| | | catcccgggatgagctgaccaagaaccaggtcagcctgacctgcctggtcaaaggcttctatcccagcgaca |
| | | tcgccgtggagtgggagagcaatgggcagccggagaacaactacaagaccacgcctcccgtgctggactccg |
| | | acggctccttcttcctctacagcaagctcaccgtggacaagagcaggtggcagcaggggaacgtcttctcat |
| | | gctccgtgatgcatgaggctctgcacaaccactacacgcagaagagcctctccctgtctccgggtaaa |
| | | SEQ ID NO: 149 |
| | | ------------------------------------------------------------------------ |
| | | -------------------------------------- |
| | | tcctgggccctgactcaacccgcctccgtgtctgcgtctcctgggcagtcggtcaccatgtcctgcactgga |
| | | ttcggaagttataatcctgactcctggtaccagcaatacccaggcaaagcccccaaactcatcatttatgat |
| | | gacaataaaagaccctcgggggtctctgatcgcttctctgcctccagacttggcagcacatcttcactgaca |
| | | atctctaacgtccaggccgctgacgacgcccattatgtctgcgcctcctttgagtttttcggcggggggacc |
| | | aagctgaccgtcctgagtcagcccaaggctgccccctcggtcactctgttcccaccctcctctgaggagctt |
| | | caagccaacaaggccacactggtgtgtctcgtaagtgacttctacccgggagccgtgacagtggcctggaag |
| | | gcagatggcagccccgtcaaggtgggagtggagaccaccaaaccctccaaacaaagcaacaacaagtatgcg |
| | | gccagcagctacctgagcctgacgcccgagcagtggaagtcccacagaagctacagctgccgggtcacgcat |
| | | gaagggagcaccgtggagaagacagtggcccctgcagaatgctct |
| | | SEQ ID NO: 150 |
| Sequence 38 | N49P72 | cacattcagttgctacagtcggggcctcaagttaagaagtctggggacacagtgagaatctcctgtgagacc |
| | | tctggatataatttcgtcgactcccttatccactgggtccgacagaccccggaaaaacgtctcagatggatg |
| | | ggctggatcaatcctctccaaggtggtgtgaattacgcgccggaatttcagggcagaatcaggatgaccagg |
| | | gacacgtttatagacacagtttacgtggacttgagcggactgacaccggccgacacggcctattattactgc |
| | | gcgcgagggatcgatggcaattcttacccctttcatttctggggccacggaacccgggtcaccgtcttctcg |
| | | gcctccaccaagggcccatcggtcttccccctggcaccctcctccaagagcacctctgggggcacagcggcc |
| | | ctgggctgcctggtcaaggactacttccccgaaccggtgacggtgtcgtggaactcaggcgccctgaccagc |
| | | ggcgtgcacaccttcccggctgtcctacagtcctcaggactctactccctcagcagcgtggtgaccgtgccc |
| | | tccagcagcttgggcacccagacctacatctgcaacgtgaatcacaagcccagcaacaccaaggtggacaag |
| | | aaagttgagcccaaatcttgtgacaaaactcacacatgcccaccgtgcccagcacctgaactcctgggggga |
| | | ccgtcagtcttcctcttccccccaaaacccaaggacaccctcatgatctcccggacccctgaggtcacatgc |
| | | gtggtggtggacgtgagccacgaagaccctgaggtcaagttcaactggtacgtggacggcgtggaggtgcat |
| | | aatgccaagacaaagccgcgggaggagcagtacaacagcacgtaccgggtggtcagcgtcctcaccgtcctg |
| | | caccaggactggctgaatggcaaggagtacaagtgcaaggtctccaacaaagccctcccagcccccatcgag |
| | | aaaaccatctccaaagccaaagggcagccccgagaaccacaggtgtacaccctgcccccatcccgggatgag |
| | | ctgaccaagaaccaggtcagcctgacctgcctggtcaaaggcttctatcccagcgacatcgccgtggagtgg |
| | | gagagcaatgggcagccggagaacaactacaagaccacgcctcccgtgctggactccgacggctccttcttc |
| | | ctctacagcaagctcaccgtggacaagagcaggtggcagcaggggaacgtcttctcatgctccgtgatgcat |
| | | gaggctctgcacaaccactacacgcagaagagcctctccctgtctccgggtaaa |
| | | SEQ ID NO: 151 |
| | | ------------------------------------------------------------------------ |
| | | -------------------------------------- |
| | | cgatttgccctgactcaacctgcctccgtgtctgggtctcctggacagacgatcaccataacctgcgctgga |
| | | ggcagcgtctcctggttccatttccctccaggcaaaacccccagactcattatttatgagtcttctaagaga |
| | | ccctcaggggtctctcctcgattctctgggtcccagtctggcagcacggcctccctaataatttctggcctc |
| | | cagtctgatgacgaagggacatacttctgttctattcttgaatttttcggcagagggactcttctcaccgtc |
| | | ctgagtcagcccaaggctgccccctcggtcactctgttcccaccctcctctgaggagcttcaagccaacaag |
| | | gccacactggtgtgtctcgtaagtgacttctacccgggagccgtgacagtggcctggaaggcagatggcagc |
| | | cccgtcaaggtgggagtggagaccaccaaaccctccaaacaaagcaacaacaagtatgcggccagcagctac |
| | | ctgagcctgacgcccgagcagtggaagtcccacagaagctacagctgccgggtcacgcatgaagggagcacc |
| | | gtggagaagacagtggcccctgcagaatgctct |
| | | SEQ ID NO: 152 |

TABLE 3
List of mAb variants and the corresponding sequence and mutations. The amino acid
sequence of the variants is determined based on the reference antibody sequence (see
table 2, above for reference antibody sequences) and the mutations described in the
table. See amino acid sequences and corresponding oligonucleotide sequences.

| | Related to | Heavy chain | Light chain | |
|---|---|---|---|---|
| mAb | sequence | mutation | mutations | Swaps/Other |
| N49P6 | 1 | CH1: 1.4A, 120R | Constant: 1.5G | LC7 swap |
| | | CH3: 12 E, 14M | | |
| N49P6.2 | 1 | CH1: 120R | | |
| | | CH3: 12 E, 14M | | |
| N49P7 | 2 | CH1: 1.4A, 120R | Constant: 1.5G | |
| | | CH3: 12 E, 14M | | |
| N49P7.1 | 2 | CH1: 120R | | |
| | | CH3: 12 E, 14M | | |
| N49P7A | 2 | CH1: 1.4A, 120R | Variable: 42A | |
| | | CH3: 12 E, 14M | Constant: 1.5G | |
| N49P7F | 2 | CH1: 1.4A, 120R | Variable: 42F | |
| | | CH3: 12 E, 14M | Constant: 1.5G | |
| N49P7S | 2 | CH1: 1.4A, 120R | Variable: 42S | |
| | | CH3: 12 E, 14M | Constant: 1.5G | |
| N49P7Y | 2 | CH1: 1.4A, 120R | Variable: 42Y | |
| | | CH3: 12 E, 14M | Constant: 1.5G | |
| N49P7-54TY | 2 | Variable: 59T/62Y | Constant: 1.5G | |
| | | CH1: 1.4A, 120R | | |
| | | CH3: 12 E, 14M | | |
| N49P7LS-1 | 2 | CH1: 1.4A, 120R | Constant: 1.5G | |
| | | CH3: 12 E, 14M, | | |
| | | 107L, 114S | | |
| N49P7LS-2 | 2 | CH1: 1.4A, 120R | Constant: 1.5G | |
| | | CH3: 107L, 114S | | |
| N49P7YTE | 2 | CH1: 1.4A, 120R | Constant: 1.5G | |
| | | CH2: 15.1Y, | | |
| | | 16T, 18E | | |
| N49P7L6 | 2.1 | CH1: 1.4A, 120R | Constant: 1.5G | N49P7 heavy chain |
| | | CH3: 12 E, 14M | | with N49P6 light |
| | | | | chain |
| N49P7L11 | 2.4 | CH1: 1.4A, 120R | | N49P7 heavy chain |
| | | CH3: 12 E, 14M | | with N49P11 light |
| | | | | chain |
| N49P7.1L9 | 2.18 | CH1: 120R | | N49P7.1 heavy chain |
| | | CH3: 12 E, 14M | | with N49P9 light |
| | | | | chain |
| N49P7.1L19 | 2.7 | CH1: 120R | | N49P7 heavy chain |
| | | CH3: 12 E, 14M | | with N49P19 light |
| | | | | chain |
| R49P7 | 2 | CH1: 1.4A | Constant: 1.5G | Rhesus IgG1, Rhesus |
| | | | | LC3 |
| N49P7.2 | 3 | CH1: 120R | | |
| | | CH3: 12 E, 14M | | |
| N49P11 | 4 | CH1: 120R | | |
| | | CH3: 12 E, 14M | | |
| N49P18 | 5 | CH1: 120R | | |
| | | CH3: 12 E, 14M | | |
| N49P18.2 | 5 | CH1: 120R | | LC7 swap |
| | | CH3: 12 E, 14M | | |
| N49P18.1 | 6 | CH1: 120R | | |
| | | CH3: 12 E, 14M | | |
| N49P19 | 7 | CH1: 120R | | |
| | | CH3: 12 E, 14M | | |
| N49P37 | 8 | CH1: 1.4A,120R | Constant: 1.5G | |
| | | CH3: 12 E, 14M | | |
| N49P38 | 9 | CH1: 120R | Constant: 1.5G | |
| | | CH3: 12 E, 14M | | |
| N4938.1 | 9 | CH1: 120R | | |
| | | CH3: 12 E, 14M | | |
| N49P55 | 10 | CH1: 120R | | |
| | | CH3: 12 E, 14M | | |
| N49P56 | 11 | CH1: 120R | | |
| | | CH3: 12 E, 14M | | |
| N49P57 | 12 | CH1: 120R | | |
| | | CH3: 12 E, 14M | | |
| N49P58 | 13 | CH1: 120R | | |
| | | CH3: 12 E, 14M | | |
| N49P59 | 14 | CH1: 120R | | |
| | | CH3: 12 E, 14M | | |
| N49P73 | 15 | CH1: 120R | | |
| | | CH3: 12 E, 14M | | |
| N49P74 | 16 | CH1: 120R | | |
| | | CH3: 12 E, 14M | | |
| N49P75 | 17 | CH1: 120R | | |
| | | CH3: 12 E, 14M | | |
| N49P75.1 | 17 | CH1: 1.4A, 120R | Constant: 1.5G | |
| | | CH3: 12 E, 14M | | |
| N49P9 | 18 | CH1: 120R | | |
| | | CH3: 12 E, 14M | | |
| N49P9.1 | 18 | CH1: 120R | | LC2 swap |
| | | CH3: 12 E, 14M | | |
| N49P9.2 | 18 | CH1: 1.4A,120R | Constant: 1.5G | |
| | | CH3: 12 E, 14M | | |
| N49P9i7 | 18.2 | CH1: 120R | | Part of CDRH3 of |
| | | CH3: 12 E, 14M | | N49P7 swapped for |
| | | | | entire CDRH3 of |
| | | | | N49P9 |
| N49P9i7H1 | 18.2 | CH1: 120R | | CDRH3 of N49P7 |
| | | CH3: 12 E, 14M | | swapped for CDRH3 |
| | | | | of N49P9 |
| N49P9i7H2 | 18.2 | CH1: 1.4S, 120R | Constant: 1.5R | CDRH3 and junction |
| | | CH3: 12 E, 14M | | of N49P7 swapped for |
| | | | | CDRH3 of N49P9 |
| N49P22 | 19 | CH1: 120R | | |
| | | CH3: 12 E, 14M | | |
| N49P23 | 20 | CH1: 120R | | |
| | | CH3: 12 E, 14M | | |
| N49P9.3 | 21 | CH1: 120R | Constant: 1.5R | |
| | | CH3: 12 E, 14M | | |
| N49P9.4 | 21 | CH1: 120R | | |
| | | CH3: 12 E, 14M | | |
| N49P51 | 22 | CH1: 120R | | |
| | | CH3: 12 E, 14M | | |
| N49P52 | 23 | CH1: 120R | | |
| | | CH3: 12 E, 14M | | |
| N49P53 | 24 | CH1: 120R | | |
| | | CH3: 12 E, 14M | | |
| N49P54 | 25 | CH1: 120R | | |
| | | CH3: 12 E, 14M | | |
| N49P60 | 26 | CH1: 120R | | |
| | | CH3: 12 E, 14M | | |
| N49P61 | 27 | CH1: 120R | | |
| | | CH3: 12 E, 14M | | |
| N49P62 | 28 | CH1: 120R | | |
| | | CH3: 12 E, 14M | | |
| N49P63 | 29 | CH1: 120R | | |
| | | CH3: 12 E, 14M | | |
| N49P64 | 30 | CH1: 120R | | |
| | | CH3: 12 E, 14M | | |
| N49P65 | 31 | CH1: 120R | | |
| | | CH3: 12 E, 14M | | |
| N49P66 | 32 | CH1: 120R | | |
| | | CH3: 12 E, 14M | | |
| N49P67 | 33 | CH1: 120R | | |
| | | CH3: 12 E, 14M | | |
| N49P68 | 34 | CH1: 120R | | |
| | | CH3: 12 E, 14M | | |
| N49P69 | 35 | CH1: 120R | | |
| | | CH3: 12 E, 14M | | |
| N49P7-FR | 2 | CH1: 1.4A, 120R | Constant: 1.5G | QLSQDPDDPDWG |
| | | CH3: 12 E, 14M | | insertion FR3 in 81-85 |
| N49P9-FR | 18 | CH1: 120R | | QLSQDPDDPDWG |
| | | CH3: 12 E, 14M | | insertion FR3 in 81-85 |
| N49P9.3-FR | 21 | CH1: 120R | Constant: 1.5R | QLSQDPDDPDWG |
| | | CH3: 12 E, 14M | | insertion FR3 in 81-85 |
| N49P9.6-FR | 21 | CH1: 1.4A, 120R | Constant: 1.5G | QLSQDPDDPDWG |
| | | CH3: 12 E, 14M | | insertion FR3 in 81-85 |
| N49P9.6-FR-54W | 21 | Variable: 59W | Constant: 1.5G | QLSQDPDDPDWG |
| | | CH1: 1.4A, 120R | | insertion FR3 in 81-85 |
| | | CH3: 12 E, 14M | | |
| N49P9.6-FR-54-F | 21 | Variable: 59F | Constant: 1.5G | QLSQDPDDPDWG |
| | | CH1: 1.4A, 120R | | insertion FR3 in 81-85 |
| | | CH3: 12 E, 14M | | |
| N49P9.6-54W | 21 | Variable: 59W | Constant: 1.5G | |
| | | CH1: 1.4A, 120R | | |
| | | CH3: 12 E, 14M | | |
| N49P9.6-54F | 21 | Variable: 59F | Constant: 1.5G | |
| | | CH1: 1.4A, 120R | | |
| | | CH3: 12 E, 14M | | |
| N49P9.6-FR3-06 | 21 | CH1: 1.4A, 120R | Constant: 1.5G | LFSQDLYYPDRG |
| | | CH3: 12 E, 14M | | insertion FR3 in 81-85 |
| N49P9.6-FR1-D | 21 | Variable: 29D | Constant: 1.5G | |
| | | CH1: 1.4A, 120R | | |
| | | CH3: 12 E, 14M | | |
| N49P9.6-FR1-D-I | 21 | Variable: 29D35I | Constant: 1.5G | |
| | | CH1: 1.4A, 120R | | |
| | | CH3: 12 E, 14M | | |
| N49P9.6-LS | 21 | CH1: 1.4A, 120R | Constant: 1.5G | |
| | | CH3: 12 E, 14M | | |
| | | 107L, 114S | | |
| N49P9.6-YTE | 21 | CH1: 1.4A, 120R | Constant: 1.5G | |
| | | CH2: 15.1Y, | | |
| | | 16T, 18E | | |
| | | CH3: 12 E, 14M | | |
| N49P9.6-FR-LS | 21 | CH1: 1.4A, 120R | Constant: 1.5G | QLSQDPDDPDWG |
| | | CH3: 12 E, 14M | | insertion FR3 in 81-85 |
| | | 107L, 114S | | |
| N49P9.6-FR-YTE | 21 | CH1: 1.4A, 120R | Constant: 1.5G | QLSQDPDDPDWG |
| | | CH2: 15.1Y, | | insertion FR3 in 81-85 |
| | | 16T, 18E | | |
| | | CH3: 12 E, 14M | | |

In some embodiments, the above listed antibodies can be further modified.

In some embodiments, modifications can be made to improve antibody function (binding, neutralization, complement fixation, ADCC, ADCP).

In some embodiments, modifications to the heavy chain can be made: Dual substitution at positions 59 and 62 to T and Y, respectively. Substitution at position 1.4 of CH1 (1^{st} amino acid of the constant region) to G from P or S. Substitution at position 120 of CH1 to R. Substitution at position 12 of CH3 to E, at position 14 of CH3 to M.

In some embodiments, light chain modifications can be made: Substitution at position 1.5 of the light chain constant region (1^{st} amino acid of the constant region) from R to S or G for those that use LC2, or from S to R or G for those that use LC7.

In some embodiments, modifications to improve antibody half-life can be made. These include the well-recognized “LS” (107L, 114S in CH3) and “YTE” (15.1Y, 16 mutations in the Fc. These also include other modifications in the Heavy chain such as: Substitution at position 120 of CH1 to R; Substitution at position 12 of CH3 to E, at position 14 of CH3 to M; Substitution at position 107 of CH3 to L, position 113 of CH3 to S, position 115 to R.

In some embodiments, modifications can be made that involve constant region swapping: Swapping of light chain constant region: light chain constant region swapped to another lambda constant region (LC1-7) for improved stability and function. Another method of swapping that can be used to make monoclonals that are “rhesus-ized,” that is, retain the variable regions in the heavy and light chains, but use constant regions from rhesus lambda chain (such as LC3), as well as rhesus IgG1. These mAbs can also have the half-life extending mutations noted above in the corresponding amino acids of the rhesus IgG1 constant region.

In some embodiments, the swapping of heavy and light chain variable regions can be made: These include taking a native or modified antibody listed in this application and mixing and matching the heavy and light chains from another in his application or N49P series to improve antibody stability or function.

In some embodiments, CDR replacement mutants can be made: These include taking the CDRH1, CDRH2, CDRH3, CDRL1, CDRL2, and/or CDRL3 from one of the parental or modified N49P series antibodies or those in this application and inserting into the corresponding CDR another natural or modified antibody.

In some embodiments, bispecific and trispecific antibodies can be made, For example, an antibody listed in this application would constitute one arm of an IgG molecule, while the other arm(s) would be another anti-HIV monoclonal (or a monoclonal that binds to a cell surface receptor). In some embodiments, the other anti-HIV monoclonal is another antibody listed in this application.

In some embodiments, antibody-drug conjugates can be made: The natural and modified antibodies listed here can be conjugated (covalently or non-covalently) to markers (florescent dye or radionuclides), therapeutic agents, or toxins such as auristatin (a microtubule toxin).

In some embodiments, pharmaceutical compositions can be made: These can include antibodies (pre-made in a variety of vehicles) delivered via injection, delivered in a slow-release depot, or genes encoding antibodies delivered via a viral or other vector.

In some embodiments, antibody-bead conjugates can be made. The modified antibodies listed here can be conjugated to agarose or other beads by a variety of chemical reactions (such as but not limited to Cyanogen Bromide-Activated and NHS esters) for the purpose of creating affinity purification columns that can bind (and purify) gp120 and its mutants, gp160 and its mutants, HIV trimers, or HIV-1 virus.

Amino acid and nucleotide sequences of the anti-HIV variant and modified antibodies are shown below. Variable regions within the heavy and light chain in the amino acid sequence are shaded and changes to the amino acid sequence relative to the natural or parental antibody sequence are underlined. CDR residues are in bold.

>N49P6
HEAVY CHAIN
SEQ ID NO: 153

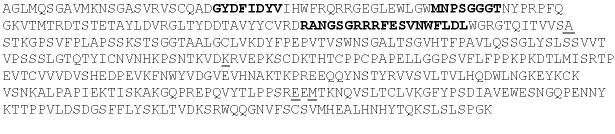

SEQ ID NO: 154
GCGGGACTGATGCAGTCTGGGGCTGTGATGAAGAATTCGGGGGCCTCAGTGAGGGTCTCTTGTCA
GGCTGATGGATACGACTTCATTGACTATGTCATTCACTGGTTTCGACAAAGACGTGGAGAAGGTCT
TGAGTGGCTGGGATGGATGAATCCCTCGGGAGGCGGCACAAACTATCCGCGACCATTTCAGGGCA
AAGTCACCATGACCAGGGACACGTCCACCGAGACAGCCTATTTAGATGTCAGAGGACTTACATAT
GACGACACGGCCGTCTATTATTGTGTGAGAGACAGGGCCAACGGTTCGGGAAGAAGACGTTTTGA
GTCGGTGAATTGGTTCCTGGATCTGTGGGGCCGCGGCACCCAAATAACAGTCGTCTCGGCCTCCAC
CAAGGGCCCATCGGTCTTCCCCCTGGCACCCTCCTCCAAGAGCACCTCTGGGGGCACAGCGGCCCT
GGGCTGCCTGGTCAAGGACTACTTCCCCGAACCGGTGACGGTGTCGTGGAACTCAGGCGCCCTGA
CCAGCGGCGTGCACACCTTCCCGGCTGTCCTACAGTCCTCAGGACTCTACTCCCTCAGCAGCGTGG
TGACCGTGCCCTCCAGCAGCTTGGGCACCCAGACCTACATCTGCAACGTGAATCACAAGCCCAGC
AACACCAAGGTGGACAAGAGAGTTGAGCCCAAATCTTGTGACAAAACTCACACATGCCCACCGTG
CCCAGCACCTGAACTCCTGGGGGGACCGTCAGTCTTCCTCTTCCCCCCAAAACCCAAGGACACCCT
CATGATCTCCCGGACCCCTGAGGTCACATGCGTGGTGGTGGACGTGAGCCACGAAGACCCTGAGG
TCAAGTTCAACTGGTACGTGGACGGCGTGGAGGTGCATAATGCCAAGACAAAGCCGCGGGAGGA
GCAGTACAACAGCACGTACCGGGTGGTCAGCGTCCTCACCGTCCTGCACCAGGACTGGCTGAATG
GCAAGGAGTACAAGTGCAAGGTCTCCAACAAAGCCCTCCCAGCCCCCATCGAGAAAACCATCTCC
AAAGCCAAAGGGCAGCCCCGAGAACCACAGGTGTACACCCTGCCCCCATCCCGGGAGGAGATGA
CCAAGAACCAGGTCAGCCTGACCTGCCTGGTCAAAGGCTTCTATCCCAGCGACATCGCCGTGGAG
TGGGAGAGCAATGGGCAGCCGGAGAACAACTACAAGACCACGCCTCCCGTGCTGGACTCCGACG
GCTCCTTCTTCCTCTACAGCAAGCTCACCGTGGACAAGAGCAGGTGGCAGCAGGGGAACGTCTTC
TCATGCTCCGTGATGCATGAGGCTCTGCACAACCACTACACGCAGAAGAGCCTCTCCCTGTCTCCG
GGTAAA
LIGHT CHAIN
SEQ ID NO: 155

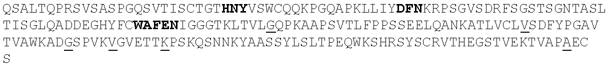

SEQ ID NO: 156
CAGTCTGCCCTAACTCAGCCTCGCTCAGTGTCCGCATCTCCTGGTCAGTCAGTCACCATCTCCTGC
ACTGGAACACACAATTATGTGTCCTGGTGTCAACAGAAACCGGGCCAAGCCCCCAAATTATTAAT
TTACGATTTCAATAAACGGCCCTCAGGGGTCTCTGATCGCTTCTCTGGCTCCACGTCTGGCAACAC
GGCCTCCCTGACCATCTCTGGACTCCAGGCTGACGATGAGGGTCATTATTTTTGTTGGGCGTTTGA
AAATATCGGCGGAGGGACCAAGCTGACCGTCCTGGGTCAGCCCAAGGCTGCCCCCTCGGTCACTC
TGTTCCCACCCTCCTCTGAGGAGCTTCAAGCCAACAAGGCCACACTGGTGTGTCTCGTAAGTGACT
TCTACCCGGGAGCCGTGACAGTGGCCTGGAAGGCAGATGGCAGCCCCGTCAAGGTGGGAGTGGA
GACCACCAAACCCTCCAAACAAAGCAACAACAAGTATGCGGCCAGCAGCTACCTGAGCCTGACGC
CCGAGCAGTGGAAGTCCCACAGAAGCTACAGCTGCCGGGTCACGCATGAAGGGAGCACCGTGGA
GAAGACAGTGGCCCCTGCAGAATGCTCT
>N49P6.2
HEAVY CHAIN
SEQ ID NO: 157

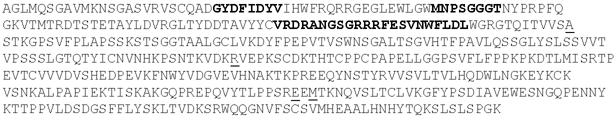

SEQ ID NO: 158
GCGGGACTGATGCAGTCTGGGGCTGTGATGAAGAATTCGGGGGCCTCAGTGAGGGTCTCTTGTCA
GGCTGATGGATACGACTTCATTGACTATGTCATTCACTGGTTTCGACAAAGACGTGGAGAAGGTCT
TGAGTGGCTGGGATGGATGAATCCCTCGGGAGGCGGCACAAACTATCCGCGACCATTTCAGGGCA
AAGTCACCATGACCAGGGACACGTCCACCGAGACAGCCTATTTAGATGTCAGAGGACTTACATAT
GACGACACGGCCGTCTATTATTGTGTGAGAGACAGGGCCAACGGTTCGGGAAGAAGACGTTTTGA
GTCGGTGAATTGGTTCCTGGATCTGTGGGGCCGCGGCACCCAAATAACAGTCGTCTCGGCCTCCAC
CAAGGGCCCATCGGTCTTCCCCCTGGCACCCTCCTCCAAGAGCACCTCTGGGGGCACAGCGGCCCT
GGGCTGCCTGGTCAAGGACTACTTCCCCGAACCGGTGACGGTGTCGTGGAACTCAGGCGCCCTGA
CCAGCGGCGTGCACACCTTCCCGGCTGTCCTACAGTCCTCAGGACTCTACTCCCTCAGCAGCGTGG
TGACCGTGCCCTCCAGCAGCTTGGGCACCCAGACCTACATCTGCAACGTGAATCACAAGCCCAGC
AACACCAAGGTGGACAAGAGAGTTGAGCCCAAATCTTGTGACAAAACTCACACATGCCCACCGTG
CCCAGCACCTGAACTCCTGGGGGGACCGTCAGTCTTCCTCTTCCCCCCAAAACCCAAGGACACCCT
CATGATCTCCCGGACCCCTGAGGTCACATGCGTGGTGGTGGACGTGAGCCACGAAGACCCTGAGG
TCAAGTTCAACTGGTACGTGGACGGCGTGGAGGTGCATAATGCCAAGACAAAGCCGCGGGAGGA
GCAGTACAACAGCACGTACCGGGTGGTCAGCGTCCTCACCGTCCTGCACCAGGACTGGCTGAATG
GCAAGGAGTACAAGTGCAAGGTCTCCAACAAAGCCCTCCCAGCCCCCATCGAGAAAACCATCTCC
AAAGCCAAAGGGCAGCCCCGAGAACCACAGGTGTACACCCTGCCCCCATCCCGGGAGGAGATGA
CCAAGAACCAGGTCAGCCTGACCTGCCTGGTCAAAGGCTTCTATCCCAGCGACATCGCCGTGGAG
TGGGAGAGCAATGGGCAGCCGGAGAACAACTACAAGACCACGCCTCCCGTGCTGGACTCCGACG
GCTCCTTCTTCCTCTACAGCAAGCTCACCGTGGACAAGAGCAGGTGGCAGCAGGGGAACGTCTTC
TCATGCTCCGTGATGCATGAGGCTCTGCACAACCACTACACGCAGAAGAGCCTCTCCCTGTCTCCG
GGTAAA
LIGHT CHAIN
SEQ ID NO: 159

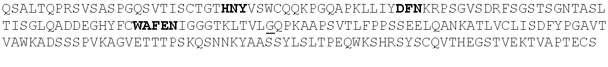

SEQ ID NO: 160
CAGTCTGCCCTAACTCAGCCTCGCTCAGTGTCCGCATCTCCTGGTCAGTCAGTCACCATCTCCTGC
ACTGGAACACACAATTATGTGTCCTGGTGTCAACAGAAACCGGGCCAAGCCCCCAAATTATTAAT
TTACGATTTCAATAAACGGCCCTCAGGGGTCTCTGATCGCTTCTCTGGCTCCACGTCTGGCAACAC
GGCCTCCCTGACCATCTCTGGACTCCAGGCTGACGATGAGGGTCATTATTTTTGTTGGGCGTTTGA
AAATATCGGCGGAGGGACCAAGCTGACCGTCCTGGGTCAGCCCAAGGCTGCCCCCTCGGTCACTC
TGTTCCCGCCCTCCTCTGAGGAGCTTCAAGCCAACAAGGCCACACTGGTGTGTCTCATAAGTGACT
TCTACCCGGGAGCCGTGACAGTGGCTTGGAAAGCAGATAGCAGCCCCGTCAAGGCGGGAGTGGA
GACCACCACACCCTCCAAACAAAGCAACAACAAGTACGCGGCCAGCAGCTATCTGAGCCTGACGC
CTGAGCAGTGGAAGTCCCACAGAAGCTACAGCTGCCAGGTCACGCATGAAGGGAGCACCGTGGA
GAAGACAGTGGCCCCTACAGAATGTTCA
>N49P7
HEAVY CHAIN
SEQ ID NO: 161

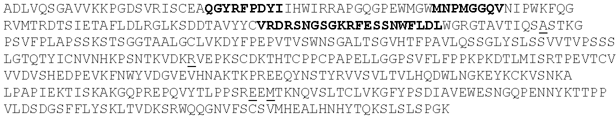

SEQ ID NO: 162
GCGGACTTGGTGCAGTCTGGGGCTGTGGTGAAGAAGCCTGGGGACTCAGTGAGGATCTCCTGTGA
GGCTCAAGGATATAGATTTCCTGACTACATCATTCACTGGATTCGACGGGCCCCTGGACAAGGCCC
TGAATGGATGGGATGGATGAATCCAATGGGCGGACAAGTAAATATTCCATGGAAATTTCAGGGTA
GGGTCTCCATGACCCGGGACACGTCCATCGAAACAGCATTTCTGGACTTAAGAGGACTAAAGTCT
GACGACACGGCCGTCTATTATTGCGTGAGAGATCGCAGTAATGGATCGGGAAAGCGATTCGAGTC
CTCCAATTGGTTCCTCGATCTGTGGGGCCGTGGGACTGCGGTCACAATTCAATCAGCCTCCACCAA
GGGCCCATCGGTCTTCCCCCTGGCACCCTCCTCCAAGAGCACCTCTGGGGGCACAGCGGCCCTGG
GCTGCCTGGTCAAGGACTACTTCCCCGAACCGGTGACGGTGTCGTGGAACTCAGGCGCCCTGACC
AGCGGCGTGCACACCTTCCCGGCTGTCCTACAGTCCTCAGGACTCTACTCCCTCAGCAGCGTGGTG
ACCGTGCCCTCCAGCAGCTTGGGCACCCAGACCTACATCTGCAACGTGAATCACAAGCCCAGCAA
CACCAAGGTGGACAAGAGAGTTGAGCCCAAATCTTGTGACAAAACTCACACATGCCCACCGTGCC
CAGCACCTGAACTCCTGGGGGGACCGTCAGTCTTCCTCTTCCCCCCAAAACCCAAGGACACCCTCA
TGATCTCCCGGACCCCTGAGGTCACATGCGTGGTGGTGGACGTGAGCCACGAAGACCCTGAGGTC
AAGTTCAACTGGTACGTGGACGGCGTGGAGGTGCATAATGCCAAGACAAAGCCGCGGGAGGAGC
AGTACAACAGCACGTACCGGGTGGTCAGCGTCCTCACCGTCCTGCACCAGGACTGGCTGAATGGC
AAGGAGTACAAGTGCAAGGTCTCCAACAAAGCCCTCCCAGCCCCCATCGAGAAAACCATCTCCAA
AGCCAAAGGGCAGCCCCGAGAACCACAGGTGTACACCCTGCCCCCATCCCGGGAGGAGATGACC
AAGAACCAGGTCAGCCTGACCTGCCTGGTCAAAGGCTTCTATCCCAGCGACATCGCCGTGGAGTG
GGAGAGCAATGGGCAGCCGGAGAACAACTACAAGACCACGCCTCCCGTGCTGGACTCCGACGGC
TCCTTCTTCCTCTACAGCAAGCTCACCGTGGACAAGAGCAGGTGGCAGCAGGGGAACGTCITTCTC
ATGCTCCGTGATGCATGAGGCTCTGCACAACCACTACACGCAGAAGAGCCTCTCCCTGTCTCCGGG
TAAA
LIGHT CHAIN
SEQ ID NO: 163

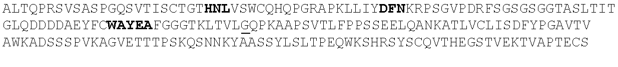

SEQ ID NO: 164
GCCCTGACTCAGCCTCGCTCAGTGTCCGCATCTCCTGGGCAGTCCGTCACCATTTCCTGCACTGGA
ACCCACAATTTGGTCTCTTGGTGTCAACATCAGCCAGGCAGAGCCCCCAAATTATTAATTTATGAC
TTCAATAAGAGGCCCTCAGGGGTCCCTGATCGCTTCTCTGGCTCCGGGTCTGGCGGCACGGCCTCC
CTGACCATCACTGGACTCCAGGATGACGATGACGCCGAATATTTTTGTTGGGCGTATGAAGCTTTT
GGCGGAGGGACCAAGTTGACCGTTCTTGGTCAGCCCAAGGCTGCCCCCTCGGTCACTCTGTTCCCG
CCCTCCTCTGAGGAGCTTCAAGCCAACAAGGCCACACTGGTGTGTCTCATAAGTGACTTCTACCCG
GGAGCCGTGACAGTGGCTTGGAAAGCAGATAGCAGCCCCGTCAAGGCGGGAGTGGAGACCACCA
CACCCTCCAAACAAAGCAACAACAAGTACGCGGCCAGCAGCTATCTGAGCCTGACGCCTGAGCAG
TGGAAGTCCCACAGAAGCTACAGCTGCCAGGTCACGCATGAAGGGAGCACCGTGGAGAAGACAG
TGGCCCCTACAGAATGTTCA
>N49P7.1
HEAVY CHAIN
SEQ ID NO: 165

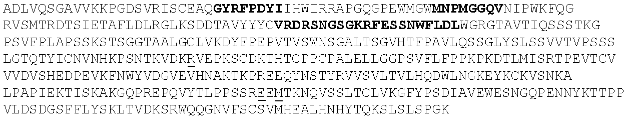

SEQ ID NO: 166
GCGGACTTGGTGCAGTCTGGGGCTGTGGTGAAGAAGCCTGGGGACTCAGTGAGGATCTCCTGTGA
GGCTCAAGGATATAGATTTCCTGACTACATCATTCACTGGATTCGACGGGCCCCTGGACAAGGCCC
TGAATGGATGGGATGGATGAATCCAATGGGCGGACAAGTAAATATTCCATGGAAATTTCAGGGTA
GGGTCTCCATGACCCGGGACACGTCCATCGAAACAGCATTTCTGGACTTAAGAGGACTAAAGTCT
GACGACACGGCCGTCTATTATTGCGTGAGAGATCGCAGTAATGGATCGGGAAAGCGATTCGAGTC
CTCCAATTGGTTCCTCGATCTGTGGGGCCGTGGGACTGCGGTCACAATTCAATCATCCTCCACCAA
GGGCCCATCGGTCTTCCCCCTGGCACCCTCCTCCAAGAGCACCTCTGGGGGCACAGCGGCCCTGG
GCTGCCTGGTCAAGGACTACTTCCCCGAACCGGTGACGGTGTCGTGGAACTCAGGCGCCCTGACC
AGCGGCGTGCACACCTTCCCGGCTGTCCTACAGTCCTCAGGACTCTACTCCCTCAGCAGCGTGGTG
ACCGTGCCCTCCAGCAGCTTGGGCACCCAGACCTACATCTGCAACGTGAATCACAAGCCCAGCAA
CACCAAGGTGGACAAGAGAGTTGAGCCCAAATCTTGTGACAAAACTCCACATGCCCACCGTGCC
CAGCACCTGAACTCCTGGGGGGACCGTCAGTCTTCCTCTTCCCCCCAAAACCCAAGGACACCCTCA
TGATCTCCCGGACCCCTGAGGTCACATGCGTGGTGGTGGACGTGAGCCACGAAGACCCTGAGGTC
AAGTTCAACTGGTACGTGGACGGCGTGGAGGTGCATAATGCCAAGACAAAGCCGCGGGAGGAGC
AGTACAACAGCACGTACCGGGTGGTCAGCGTCCTCACCGTCCTGCACCAGGACTGGCTGAATGGC
AAGGAGTACAAGTGCAAGGTCTCCAACAAAGCCCTCCCAGCCCCCATCGAGAAAACCATCTCCAA
AGCCAAAGGGCAGCCCCGAGAACCACAGGTGTACACCCTGCCCCCATCCCGGGAGGAGATGACC
AAGAACCAGGTCAGCCTGACCTGCCTGGTCAAAGGCTTCTATCCCAGCGACATCGCCGTGGAGTG
GGAGAGCAATGGGCAGCCGGAGAACAACTACAAGACCACGCCTCCCGTGCTGGACTCCGACGGC
TCCTTCTTCCTCTACAGCAAGCTCACCGTGGACAAGAGCAGGTGGCAGCAGGGGAACGTCTTCTC
ATGCTCCGTGATGCATGAGGCTCTGCACAACCACTACACGCAGAAGAGCCTCTCCCTGTCTCCGGG
TAAA
LIGHT CHAIN
SEQ ID NO: 167

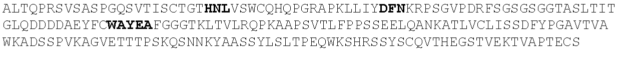

SEQ ID NO: 168
GCCCTGACTCAGCCTCGCTCAGTGTCCGCATCTCCTGGGCAGTCCGTCACCATTTCCTGCACTGGA
ACCCACAATTTGGTCTCTTGGTGTCAACATCAGCCAGGCAGAGCCCCCAAATTATTAATTTATGAC
TTCAATAAGAGGCCCTCAGGGGTCCCTGATCGCTTCTCTGGCTCCGGGTCTGGCGGCACGGCCTCC
CTGACCATCACTGGACTCCAGGATGACGATGACGCCGAATATTTTTGTTGGGCGTATGAAGCTTTT
GGCGGAGGGACCAAGTTGACCGTTCTTCGTCAGCCCAAGGCTGCCCCCTCGGTCACTCTGTTCCCG
CCCTCCTCTGAGGAGCTTCAAGCCAACAAGGCCACACTGGTGTGTCTCATAAGTGACTTCTACCCG
GGAGCCGTGACAGTGGCTTGGAAAGCAGATAGCAGCCCCGTCAAGGCGGGAGTGGAGACCACCA
CACCCTCCAAACAAAGCAACAACAAGTACGCGGCCAGCAGCTATCTGAGCCTGACGCCTGAGCAG
TGGAAGTCCCACAGAAGCTACAGCTGCCAGGTCACGCATGAAGGGAGCACCGTGGAGAAGACAG
TGGCCCCTACAGAATGTTCA
>N49P7A
HEAVY CHAIN
SEQ ID NO: 169

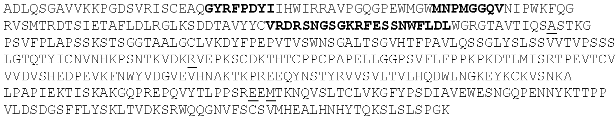

SEQ ID NO: 170
GCGGACTTGGTGCAGTCTGGGGCTGTGGTGAAGAAGCCTGGGGACTCAGTGAGGATCTCCTGTGA
GGCTCAAGGATATAGATTTCCTGACTACATCATTCACTGGATTCGACGGGCCCCTGGACAAGGCCC
TGAATGGATGGGATGGATGAATCCAATGGGCGGACAAGTAAATATTCCATGGAAATTTCAGGGTA
GGGTCTCCATGACCCGGGACACGTCCATCGAAACAGCATTTCTGGACTTAAGAGGACTAAAGTCT
GACGACACGGCCGTCTATTATTGCGTGAGAGATCGCAGTAATGGATCGGGAAAGCGATTCGAGTC
CTCCAATTGGTTCCTCGATCTGTGGGGCCGTGGGACTGCGGTCACAATTCAATCAGCCTCCACCAA
GGGCCCATCGGTCTTCCCCCTGGCACCCTCCTCCAAGAGCACCTCTGGGGGCACAGCGGCCCTGG
GCTGCCTGGTCAAGGACTACTTCCCCGAACCGGTGACGGTGTCGTGGAACTCAGGCGCCCTGACC
AGCGGCGTGCACACCTTCCCGGCTGTCCTACAGTCCTCAGGACTCTACTCCCTCAGCAGCGTGGTG
ACCGTGCCCTCCAGCAGCTTGGGCACCCAGACCTACATCTGCAACGTGAATCACAAGCCCAGCAA
CACCAAGGTGGACAAGAGAGTTGAGCCCAAATCTTGTGACAAAACTCACACATGCCCACCGTGCC
CAGCACCTGAACTCCTGGGGGGACCGTCAGTCTTCCTCTTCCCCCCAAAACCCAAGGACACCCTCA
TGATCTCCCGGACCCCTGAGGTCACATGCGTGGTGGTGGACGTGAGCCACGAAGACCCTGAGGTC
AAGTTCAACTGGTACGTGGACGGCGTGGAGGTGCATAATGCCAAGACAAAGCCGCGGGAGGAGC
AGTACAACAGCACGTACCGGGTGGTCAGCGTCCTCACCGTCCTGCACCAGGACTGGCTGAATGGC
AAGGAGTACAAGTGCAAGGTCTCCAACAAAGCCCTCCCAGCCCCCATCGAGAAAACCATCTCCAA
AGCCAAAGGGCAGCCCCGAGAACCACAGGTGTACACCCTGCCCCCATCCCGGGAGGAGATGACC
AAGAACCAGGTCAGCCTGACCTGCCTGGTCAAAGGCTTCTATCCCAGCGACATCGCCGTGGAGTG
GGAGAGCAATGGGCAGCCGGAGAACAACTACAAGACCACGCCTCCCGTGCTGGACTCCGACGGC
TCCTTCTTCCTCTACAGCAAGCTCACCGTGGACAAGAGCAGGTGGCAGCAGGGGAACGTCTTCTC
ATGCTCCGTGATGCATGAGGCTCTGCACAACCACTACACGCAGAAGAGCCTCTCCCTGTCTCCGGG
TAAA
LIGHT CHAIN
SEQ ID NO: 171

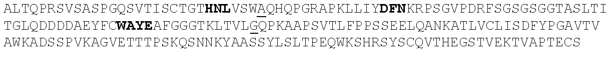

SEQ ID NO: 172
GCCCTGACTCAGCCTCGCTCAGTGTCCGCATCTCCTGGGCAGTCCGTCACCATTTCCTGCACTGGA
ACCCACAATTTGGTCTCTTGGGCTCAACATCAGCCAGGCAGAGCCCCCAAATTATTAATTTATGAC
TTCAATAAGAGGCCCTCAGGGGTCCCTGATCGCTTCTCTGGCTCCGGGTCTGGCGGCACGGCCTCC
CTGACCATCACTGGACTCCAGGATGACGATGACGCCGAATATTTTTGTTGGGCGTATGAAGCTTTT
GGCGGAGGGACCAAGTTGACCGTTCTTGGTCAGCCCAAGGCTGCCCCCTCGGTCACTCTGTTCCCG
CCCTCCTCTGAGGAGCTTCAAGCCAACAAGGCCACACTGGTGTGTCTCATAAGTGACTTCTACCCG
GGAGCCGTGACAGTGGCTTGGAAAGCAGATAGCAGCCCCGTCAAGGCGGGAGTGGAGACCACCA
CACCCTCCAAACAAAGCAACAACAAGTACGCGGCCAGCAGCTATCTGAGCCTGACGCCTGAGCAG
TGGAAGTCCCACAGAAGCTACAGCTGCCAGGTCACGCATGAAGGGAGCACCGTGGAGAAGACAG
TGGCCCCTACAGAATGTTCA
>N49P7F
HEAVY CHAIN
SEQ ID NO: 173

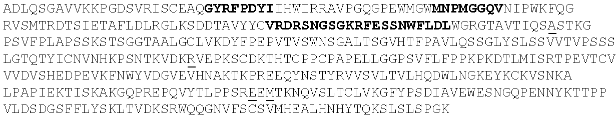

SEQ ID NO: 174
GCGGACTTGGTGCAGTCTGGGGCTGTGGTGAAGAAGCCTGGGGACTCAGTGAGGATCTCCTGTGA
GGCTCAAGGATATAGATTTCCTGACTACATCATTCACTGGATTCGACGGGCCCCTGGACAAGGCCC
TGAATGGATGGGATGGATGAATCCAATGGGCGGACAAGTAAATATTCCATGGAAATTTCAGGGTA
GGGTCTCCATGACCCGGGACACGTCCATCGAAACAGCATTTCTGGACTTAAGAGGACTAAAGTCT
GACGACACGGCCGTCTATTATTGCGTGAGAGATCGCAGTAATGGATCGGGAAAGCGATTCGAGTC
CTCCAATTGGTTCCTCGATCTGTGGGGCCGTGGGACTGCGGTCACAATTCAATCAGCCTCCACCAA
GGGCCCATCGGTCTTCCCCCTGGCACCCTCCTCCAAGAGCACCTCTGGGGGCACAGCGGCCCTGG
GCTGCCTGGTCAAGGACTACTTCCCCGAACCGGTGACGGTGTCGTGGAACTCAGGCGCCCTGACC
AGCGGCGTGCACACCTTCCCGGCTGTCCTACAGTCCTCAGGACTCTACTCCCTCAGCAGCGTGGTG
ACCGTGCCCTCCAGCAGCTTGGGCACCCAGACCTACATCTGCAACGTGAATCACAAGCCCAGCAA
CACCAAGGTGGACAAGAGAGTTGAGCCCAAATCTTGTGACAAAACTCACACATGCCCACCGTGCC
CAGCACCTGAACTCCTGGGGGGACCGTCAGTCTTCCTCTTCCCCCCAAAACCCAAGGACACCCTCA
TGATCTCCCGGACCCCTGAGGTCACATGCGTGGTGGTGGACGTGAGCCACGAAGACCCTGAGGTC
AAGTTCAACTGGTACGTGGACGGCGTGGAGGTGCATAATGCCAAGACAAAGCCGCGGGAGGAGC
AGTACAACAGCACGTACCGGGTGGTCAGCGTCCTCACCGTCCTGCACCAGGACTGGCTGAATGGC
AAGGAGTACAAGTGCAAGGTCTCCAACAAAGCCCTCCCAGCCCCCATCGAGAAAACCATCTCCAA
AGCCAAAGGGCAGCCCCGAGAACCACAGGTGTACACCCTGCCCCCATCCCGGGAGGAGATGACC
AAGAACCAGGTCAGCCTGACCTGCCTGGTCAAAGGCTTCTATCCCAGCGACATCGCCGTGGAGTG
GGAGAGCAATGGGCAGCCGGAGAACAACTACAAGACCACGCCTCCCGTGCTGGACTCCGACGGC
TCCTTCTTCCTCTACAGCAAGCTCACCGTGGACAAGAGCAGGTGGCAGCAGGGGAACGTCTTCTC
ATGCTCCGTGATGCATGAGGCTCTGCACAACCACTACACGCAGAAGAGCCTCTCCCTGTCTCCGGG
TAAA
LIGHT CHAIN
SEQ ID NO: 175

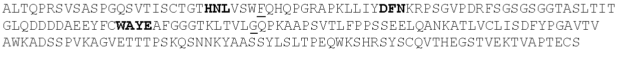

SEQ ID NO: 176
GCCCTGACTCAGCCTCGCTCAGTGTCCGCATCTCCTGGGCAGTCCGTCACCATTTCCTGCACTGGA
ACCCACAATTTGGTCTCTTGGTTTCAACATCAGCCAGGCAGAGCCCCCAAATTATTAATTTATGAC
TTCAATAAGAGGCCCTCAGGGGTCCCTGATCGCTTCTCTGGCTCCGGGTCTGGCGGCACGGCCTCC
CTGACCATCACTGGACTCCAGGATGACGATGACGCCGAATATTTTTGTTGGGCGTATGAAGCTTTT
GGCGGAGGGACCAAGTTGACCGTTCTTGGTCAGCCCAAGGCTGCCCCCTCGGTCACTCTGTTCCCG
CCCTCCTCTGAGGAGCTTCAAGCCAACAAGGCCACACTGGTGTGTCTCATAAGTGACTTCTACCCG
GGAGCCGTGACAGTGGCTTGGAAAGCAGATAGCAGCCCCGTCAAGGCGGGAGTGGAGACCACCA
CACCCTCCAAACAAAGCAACAACAAGTACGCGGCCAGCAGCTATCTGAGCCTGACGCCTGAGCAG
TGGAAGTCCCACAGAAGCTACAGCTGCCAGGTCACGCATGAAGGGAGCACCGTGGAGAAGACAG
TGGCCCCTACAGAATGTTCA
>N49P7S
HEAVY CHAIN
SEQ ID NO: 177

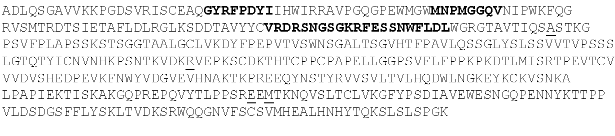

SEQ ID NO: 178
GCGGACTTGGTGCAGTCTGGGGCTGTGGTGAAGAAGCCTGGGGACTCAGTGAGGATCTCCTGTGA
GGCTCAAGGATATAGATTTCCTGACTACATCATTCACTGGATTCGACGGGCCCCTGGACAAGGCCC
TGAATGGATGGGATGGATGAATCCAATGGGCGGACAAGTAAATATTCCATGGAAATTTCAGGGTA
GGGTCTCCATGACCCGGGACACGTCCATCGAAACAGCATTTCTGGACTTAAGAGGACTAAAGTCT
GACGACACGGCCGTCTATTATTGCGTGAGAGATCGCAGTAATGGATCGGGAAAGCGATTCGAGTC
CTCCAATTGGTTCCTCGATCTGTGGGGCCGTGGGACTGCGGTCACAATTCAATCAGCCTCCACCAA
GGGCCCATCGGTCTTCCCCCTGGCACCCTCCTCCAAGAGCACCTCTGGGGGCACAGCGGCCCTGG
GCTGCCTGGTCAAGGACTACTTCCCCGAACCGGTGACGGTGTCGTGGAACTCAGGCGCCCTGACC
AGCGGCGTGCACACCTTCCCGGCTGTCCTACAGTCCTCAGGACTCTACTCCCTCAGCAGCGTGGTG
ACCGTGCCCTCCAGCAGCTTGGGCACCCAGACCTACATCTGCAACGTGAATCACAAGCCCAGCAA
CACCAAGGTGGACAAGAGAGTTGAGCCCAAATCTTGTGACAAAACTCACACATGCCCACCGTGCC
CAGCACCTGAACTCCTGGGGGGACCGTCAGTCTTCCTCTTCCCCCCAAAACCCAAGGACACCCTCA
TGATCTCCCGGACCCCTGAGGTCACATGCGTGGTGGTGGACGTGAGCCACGAAGACCCTGAGGTC
AAGTTCAACTGGTACGTGGACGGCGTGGAGGTGCATAATGCCAAGACAAAGCCGCGGGAGGAGC
AGTACAACAGCACGTACCGGGTGGTCAGCGTCCTCACCGTCCTGCACCAGGACTGGCTGAATGGC
AAGGAGTACAAGTGCAAGGTCTCCAACAAAGCCCTCCCAGCCCCCATCGAGAAAACCATCTCCAA
AGCCAAAGGGCAGCCCCGAGAACCACAGGTGTACACCCTGCCCCCATCCCGGGAGGAGATGACC
AAGAACCAGGTCAGCCTGACCTGCCTGGTCAAAGGCTTCTATCCCAGCGACATCGCCGTGGAGTG
GGAGAGCAATGGGCAGCCGGAGAACAACTACAAGACCACGCCTCCCGTGCTGGACTCCGACGGC
TCCTTCTTCCTCTACAGCAAGCTCACCGTGGACAAGAGCAGGTGGCAGCAGGGGAACGTCTTCTC
ATGCTCCGTGATGCATGAGGCTCTGCACAACCACTACACGCAGAAGAGCCTCTCCCTGTCTCCGGG
TAAA
LIGHT CHAIN
SEQ ID NO: 179

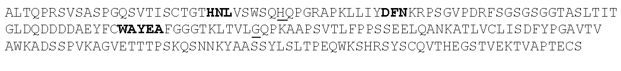

SEQ ID NO: 180
GCCCTGACTCAGCCTCGCTCAGTGTCCGCATCTCCTGGGCAGTCCGTCACCATTTCCTGCACTGGA
ACCCACAATTTGGTCTCTTGGTCTCAACATCAGCCAGGCAGAGCCCCCAAATTATTAATTTATGAC
TTCAATAAGAGGCCCTCAGGGGTCCCTGATCGCTTCTCTGGCTCCGGGTCTGGCGGCACGGCCTCC
CTGACCATCACTGGACTCCAGGATGACGATGACGCCGAATATTTTTGTTGGGCGTATGAAGCTTTT
GGCGGAGGGACCAAGTTGACCGTTCTTGGTCAGCCCAAGGCTGCCCCCTCGGTCACTCTGTTCCCG
CCCTCCTCTGAGGAGCTTCAAGCCAACAAGGCCACACTGGTGTGTCTCATAAGTGACTTCTACCCG
GGAGCCGTGACAGTGGCTTGGAAAGCAGATAGCAGCCCCGTCAAGGCGGGAGTGGAGACCACCA
CACCCTCCAAACAAAGCAACAACAAGTACGCGGCCAGCAGCTATCTGAGCCTGACGCCTGAGCAG
TGGAAGTCCCACAGAAGCTACAGCTGCCAGGTCACGCATGAAGGGAGCACCGTGGAGAAGACAG
TGGCCCCTACAGAATGTTCA
>N49P7Y
HEAVY CHAIN
SEQ ID NO: 181

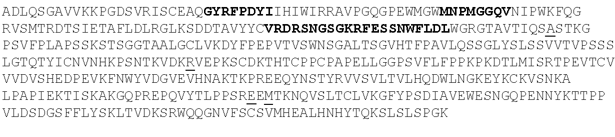

SEQ ID NO: 182
GCGGACTTGGTGCAGTCTGGGGCTGTGGTGAAGAAGCCTGGGGACTCAGTGAGGATCTCCTGTGA
GGCTCAAGGATATAGATTTCCTGACTACATCATTCACTGGATTCGACGGGCCCCTGGACAAGGCCC
TGAATGGATGGGATGGATGAATCCAATGGGCGGACAAGTAAATATTCCATGGAAATTTCAGGGTA
GGGTCTCCATGACCCGGGACACGTCCATCGAAACAGCATTTCTGGACTTAAGAGGACTAAAGTCT
GACGACACGGCCGTCTATTATTGCGTGAGAGATCGCAGTAATGGATCGGGAAAGCGATTCGAGTC
CTCCAATTGGTTCCTCGATCTGTGGGGCCGTGGGACTGCGGTCACAATTCAATCAGCCTCCACCAA
GGGCCCATCGGTCTTCCCCCTGGCACCCTCCTCCAAGAGCACCTCTGGGGGCACAGCGGCCCTGG
GCTGCCTGGTCAAGGACTACTTCCCCGAACCGGTGACGGTGTCGTGGAACTCAGGCGCCCTGACC
AGCGGCGTGCACACCTTCCCGGCTGTCCTACAGTCCTCAGGACTCTACTCCCTCAGCAGCGTGGTG
ACCGTGCCCTCCAGCAGCTTGGGCACCCAGACCTACATCTGCAACGTGAATCACAAGCCCAGCAA
CACCAAGGTGGACAAGAGAGTTGAGCCCAAATCTTGTGACAAAACTCACACATGCCCACCGTGCC
CAGCACCTGAACTCCTGGGGGGACCGTCAGTCTTCCTCTTCCCCCCAAAACCCAAGGACACCCTCA
TGATCTCCCGGACCCCTGAGGTCACATGCGTGGTGGTGGACGTGAGCCACGAAGACCCTGAGGTC
AAGTTCAACTGGTACGTGGACGGCGTGGAGGTGCATAATGCCAAGACAAAGCCGCGGGAGGAGC
AGTACAACAGCACGTACCGGGTGGTCAGCGTCCTCACCGTCCTGCACCAGGACTGGCTGAATGGC
AAGGAGTACAAGTGCAAGGTCTCCAACAAAGCCCTCCCAGCCCCCATCGAGAAAACCATCTCCAA
AGCCAAAGGGCAGCCCCGAGAACCACAGGTGTACACCCTGCCCCCATCCCGGGAGGAGATGACC
AAGAACCAGGTCAGCCTGACCTGCCTGGTCAAAGGCTTCTATCCCAGCGACATCGCCGTGGAGTG
GGAGAGCAATGGGCAGCCGGAGAACAACTACAAGACCACGCCTCCCGTGCTGGACTCCGACGGC
TCCTTCTTCCTCTACAGCAAGCTCACCGTGGACAAGAGCAGGTGGCAGCAGGGGAACGTCITTCTC
ATGCTCCGTGATGCATGAGGCTCTGCACAACCACTACACGCAGAAGAGCCTCTCCCTGTCTCCGGG
TAAA
LIGHT CHAIN
SEQ ID NO: 183

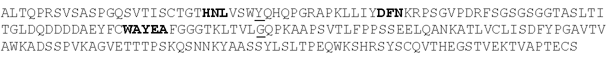

SEQ ID NO: 184
GCCCTGACTCAGCCTCGCTCAGTGTCCGCATCTCCTGGGCAGTCCGTCACCATTTCCTGCACTGGA
ACCCACAATTTGGTCTCTTGGTATCAACATCAGCCAGGCAGAGCCCCCAAATTATTAATTTATGAC
TTCAATAAGAGGCCCTCAGGGGTCCCTGATCGCTTCTCTGGCTCCGGGTCTGGCGGCACGGCCTCC
CTGACCATCACTGGACTCCAGGATGACGATGACGCCGAATATTTTTGTTGGGCGTATGAAGCTTTT
GGCGGAGGGACCAAGTTGACCGTTCTTGGTCAGCCCAAGGCTGCCCCCTCGGTCACTCTGTTCCCG
CCCTCCTCTGAGGAGCTTCAAGCCAACAAGGCCACACTGGTGTGTCTCATAAGTGACTTCTACCCG
GGAGCCGTGACAGTGGCTTGGAAAGCAGATAGCAGCCCCGTCAAGGCGGGAGTGGAGACCACCA
CACCCTCCAAACAAAGCAACAACAAGTACGCGGCCAGCAGCTATCTGAGCCTGACGCCTGAGCAG
TGGAAGTCCCACAGAAGCTACAGCTGCCAGGTCACGCATGAAGGGAGCACCGTGGAGAAGACAG
TGGCCCCTACAGAATGTTCA
>N49P7-54T7
HEAVY CHAIN
SEQ ID NO: 185

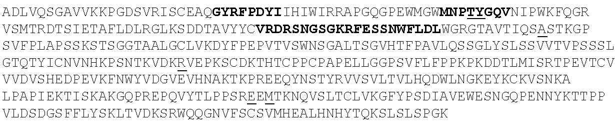

SEQ ID NO: 186
GCGGACTTGGTGCAGTCTGGGGCTGTGGTGAAGAAGCCTGGGGACTCAGTGAGGATCTCCTGTGA
GGCTCAAGGATATAGATTTCCTGACTACATCATTCACTGGATTCGACGGGCCCCTGGACAAGGCCC
TGAATGGATGGGATGGATGAATCCAACGTACGGACAAGTAAATATTCCATGGAAATTTCAGGGTA
GGGTCTCCATGACCCGGGACACGTCCATCGAAACAGCATTTCTGGACTTAAGAGGACTAAAGTCT
GACGACACGGCCGTCTATTATTGCGTGAGAGATCGCAGTAATGGATCGGGAAAGCGATTCGAGTC
CTCCAATTGGTTCCTCGATCTGTGGGGCCGTGGGACTGCGGTCACAATTCAATCAGCCTCCACCAA
GGGCCCATCGGTCTTCCCCCTGGCACCCTCCTCCAAGAGCACCTCTGGGGGCACAGCGGCCCTGG
GCTGCCTGGTCAAGGACTACTTCCCCGAACCGGTGACGGTGTCGTGGAACTCAGGCGCCCTGACC
AGCGGCGTGCACACCTTCCCGGCTGTCCTACAGTCCTCAGGACTCTACTCCCTCAGCAGCGTGGTG
ACCGTGCCCTCCAGCAGCTTGGGCACCCAGACCTACATCTGCAACGTGAATCACAAGCCCAGCAA
CACCAAGGTGGACAAGAGAGTTGAGCCCAAATCTTGTGACAAAACTCACACATGCCCACCGTGCC
CAGCACCTGAACTCCTGGGGGGACCGTCAGTCTTCCTCTTCCCCCCAAAACCCAAGGACACCCTCA
TGATCTCCCGGACCCCTGAGGTCACATGCGTGGTGGTGGACGTGAGCCACGAAGACCCTGAGGTC
AAGTTCAACTGGTACGTGGACGGCGTGGAGGTGCATAATGCCAAGACAAAGCCGCGGGAGGAGC
AGTACAACAGCACGTACCGGGTGGTCAGCGTCCTCACCGTCCTGCACCAGGACTGGCTGAATGGC
AAGGAGTACAAGTGCAAGGTCTCCAACAAAGCCCTCCCAGCCCCCATCGAGAAAACCATCTCCAA
AGCCAAAGGGCAGCCCCGAGAACCACAGGTGTACACCCTGCCCCCATCCCGGGAGGAGATGACC
AAGAACCAGGTCAGCCTGACCTGCCTGGTCAAAGGCTTCTATCCCAGCGACATCGCCGTGGAGTG
GGAGAGCAATGGGCAGCCGGAGAACAACTACAAGACCACGCCTCCCGTGCTGGACTCCGACGGC
TCCTTCTTCCTCTACAGCAAGCTCACCGTGGACAAGAGCAGGTGGCAGCAGGGGAACGTCTTCTC
ATGCTCCGTGATGCATGAGGCTCTGCACAACCACTACACGCAGAAGAGCCTCTCCCTGTCTCCGGG
TAAA
LIGHT CHAIN
SEQ ID NO: 187

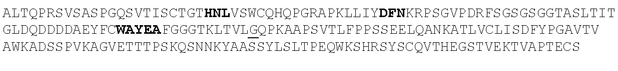

SEQ ID NO: 188
GCCCTGACTCAGCCTCGCTCAGTGTCCGCATCTCCTGGGCAGTCCGTCACCATTTCCTGCACTGGA
ACCCACAATTTGGTCTCTTGGTGTCAACATCAGCCAGGCAGAGCCCCCAAATTATTAATTTATGAC
TTCAATAAGAGGCCCTCAGGGGTCCCTGATCGCTTCTCTGGCTCCGGGTCTGGCGGCACGGCCTCC
CTGACCATCACTGGACTCCAGGATGACGATGACGCCGAATATTTTTGTTGGGCGTATGAAGCTTTT
GGCGGAGGGACCAAGTTGACCGTTCTTGGTCAGCCCAAGGCTGCCCCCTCGGTCACTCTGTTCCCG
CCCTCCTCTGAGGAGCTTCAAGCCAACAAGGCCACACTGGTGTGTCTCATAAGTGACTTCTACCCG
GGAGCCGTGACAGTGGCTTGGAAAGCAGATAGCAGCCCCGTCAAGGCGGGAGTGGAGACCACCA
CACCCTCCAAACAAAGCAACAACAAGTACGCGGCCAGCAGCTATCTGAGCCTGACGCCTGAGCAG
TGGAAGTCCCACAGAAGCTACAGCTGCCAGGTCACGCATGAAGGGAGCACCGTGGAGAAGACAG
TGGCCCCTACAGAATGTTCA
>N49P7LS-1
HEAVY CHAIN
SEQ ID NO: 189

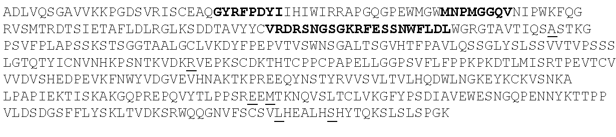

SEQ ID NO: 190
GCGGACTTGGTGCAGTCTGGGGCTGTGGTGAAGAAGCCTGGGGACTCAGTGAGGATCTCCTGTGA
GGCTCAAGGATATAGATTTCCTGACTACATCATTCACTGGATTCGACGGGCCCCTGGACAAGGCCC
TGAATGGATGGGATGGATGAATCCAATGGGCGGACAAGTAAATATTCCATGGAAATTTCAGGGTA
GGGTCTCCATGACCCGGGACACGTCCATCGAAACAGCATTTCTGGACTTAAGAGGACTAAAGTCT
GACGACACGGCCGTCTATTATTGCGTGAGAGATCGCAGTAATGGATCGGGAAAGCGATTCGAGTC
CTCCAATTGGTTCCTCGATCTGTGGGGCCGTGGGACTGCGGTCACAATTCAATCAGCCTCCACCAA
GGGCCCATCGGTCTTCCCCCTGGCACCCTCCTCCAAGAGCACCTCTGGGGGCACAGCGGCCCTGG
GCTGCCTGGTCAAGGACTACTTCCCCGAACCGGTGACGGTGTCGTGGAACTCAGGCGCCCTGACC
AGCGGCGTGCACACCTTCCCGGCTGTCCTACAGTCCTCAGGACTCTACTCCCTCAGCAGCGTGGTG
ACCGTGCCCTCCAGCAGCTTGGGCACCCAGACCTACATCTGCAACGTGAATCACAAGCCCAGCAA
CACCAAGGTGGACAAGAGAGTTGAGCCCAAATCTTGTGACAAAACTCACACATGCCCACCGTGCC
CAGCACCTGAACTCCTGGGGGGACCGTCAGTCTTCCTCTTCCCCCCAAAACCCAAGGACACCCTCA
TGATCTCCCGGACCCCTGAGGTCACATGCGTGGTGGTGGACGTGAGCCACGAAGACCCTGAGGTC
AAGTTCAACTGGTACGTGGACGGCGTGGAGGTGCATAATGCCAAGACAAAGCCGCGGGAGGAGC
AGTACAACAGCACGTACCGGGTGGTCAGCGTCCTCACCGTCCTGCACCAGGACTGGCTGAATGGC
AAGGAGTACAAGTGCAAGGTCTCCAACAAAGCCCTCCCAGCCCCCATCGAGAAAACCATCTCCAA
AGCCAAAGGGCAGCCCCGAGAACCACAGGTGTACACCCTGCCCCCATCCCGGGAGGAGATGACC
AAGAACCAGGTCAGCCTGACCTGCCTGGTCAAAGGCTTCTATCCCAGCGACATCGCCGTGGAGTG
GGAGAGCAATGGGCAGCCGGAGAACAACTACAAGACCACGCCTCCCGTGCTGGACTCCGACGGC
TCCTTCTTCCTCTACAGCAAGCTCACCGTGGACAAGAGCAGGTGGCAGCAGGGGAACGTCTTCTC
ATGCTCCGTGCTGCATGAGGCTCTGCACAGCCACTACACGCAGAAGAGCCTCTCCCTGTCCCCGGG
TAAA
LIGHT CHAIN
SEQ ID NO: 191

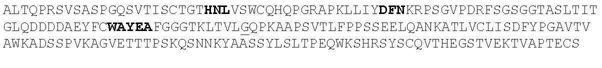

SEQ ID NO: 192
GCCCTGACTCAGCCTCGCTCAGTGTCCGCATCTCCTGGGCAGTCCGTCACCATTTCCTGCACTGGA
ACCCACAATTTGGTCTCTTGGTGTCAACATCAGCCAGGCAGAGCCCCCAAATTATTAATTTATGAC
TTCAATAAGAGGCCCTCAGGGGTCCCTGATCGCTTCTCTGGCTCCGGGTCTGGCGGCACGGCCTCC
CTGACCATCACTGGACTCCAGGATGACGATGACGCCGAATATTTTTGTTGGGCGTATGAAGCTTTT
GGCGGAGGGACCAAGTTGACCGTTCTTGGTCAGCCCAAGGCTGCCCCCTCGGTCACTCTGTTCCCG
CCCTCCTCTGAGGAGCTTCAAGCCAACAAGGCCACACTGGTGTGTCTCATAAGTGACTTCTACCCG
GGAGCCGTGACAGTGGCTTGGAAAGCAGATAGCAGCCCCGTCAAGGCGGGAGTGGAGACCACCA
CACCCTCCAAACAAAGCAACAACAAGTACGCGGCCAGCAGCTATCTGAGCCTGACGCCTGAGCAG
TGGAAGTCCCACAGAAGCTACAGCTGCCAGGTCACGCATGAAGGGAGCACCGTGGAGAAGACAG
TGGCCCCTACAGAATGTTCA
>N49P7LS-2
HEAVY CHAIN
SEQ ID NO: 193

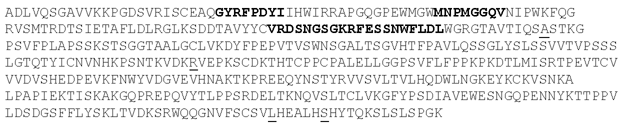

SEQ ID NO: 194
GCGGACTTGGTGCAGTCTGGGGCTGTGGTGAAGAAGCCTGGGGACTCAGTGAGGATCTCCTGTGA
GGCTCAAGGATATAGATTTCCTGACTACATCATTCACTGGATTCGACGGGCCCCTGGACAAGGCCC
TGAATGGATGGGATGGATGAATCCAATGGGCGGACAAGTAAATATTCCATGGAAATTTCAGGGTA
GGGTCTCCATGACCCGGGACACGTCCATCGAAACAGCATTTCTGGACTTAAGAGGACTAAAGTCT
GACGACACGGCCGTCTATTATTGCGTGAGAGATCGCAGTAATGGATCGGGAAAGCGATTCGAGTC
CTCCAATTGGTTCCTCGATCTGTGGGGCCGTGGGACTGCGGTCACAATTCAATCAGCCTCCACCAA
GGGCCCATCGGTCTTCCCCCTGGCACCCTCCTCCAAGAGCACCTCTGGGGGCACAGCGGCCCTGG
GCTGCCTGGTCAAGGACTACTTCCCCGAACCGGTGACGGTGTCGTGGAACTCAGGCGCCCTGACC
AGCGGCGTGCACACCTTCCCGGCTGTCCTACAGTCCTCAGGACTCTACTCCCTCAGCAGCGTGGTG
ACCGTGCCCTCCAGCAGCTTGGGCACCCAGACCTACATCTGCAACGTGAATCACAAGCCCAGCAA
CACCAAGGTGGACAAGAGAGTTGAGCCCAAATCTTGTGACAAAACTCACACATGCCCACCGTGCC
CAGCACCTGAACTCCTGGGGGGACCGTCAGTCTTCCTCTTCCCCCCAAAACCCAAGGACACCCTCA
TGATCTCCCGGACCCCTGAGGTCACATGCGTGGTGGTGGACGTGAGCCACGAAGACCCTGAGGTC
AAGTTCAACTGGTACGTGGACGGCGTGGAGGTGCATAATGCCAAGACAAAGCCGCGGGAGGAGC
AGTACAACAGCACGTACCGGGTGGTCAGCGTCCTCACCGTCCTGCACCAGGACTGGCTGAATGGC
AAGGAGTACAAGTGCAAGGTCTCCAACAAAGCCCTCCCAGCCCCCATCGAGAAAACCATCTCCAA
AGCCAAAGGGCAGCCCCGAGAACCACAGGTGTACACCCTGCCCCCATCCCGGGATGAGCTGACCA
AGAACCAGGTCAGCCTGACCTGCCTGGTCAAAGGCTTCTATCCCAGCGACATCGCCGTGGAGTGG
GAGAGCAATGGGCAGCCGGAGAACAACTACAAGACCACGCCTCCCGTGCTGGACTCCGACGGCTC
CTTCTTCCTCTACAGCAAGCTCACCGTGGACAAGAGCAGGTGGCAGCAGGGGAACGTCTTCTCAT
GCTCCGTGCTGCATGAGGCTCTGCACAGCCACTACACGCAGAAGAGCCTCTCCCTGTCCCCGGGTA
AA
LIGHT CHAIN
SEQ ID NO: 195

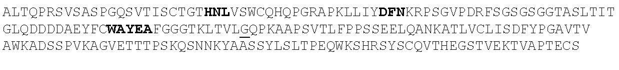

SEQ ID NO: 196
GCCCTGACTCAGCCTCGCTCAGTGTCCGCATCTCCTGGGCAGTCCGTCACCATTTCCTGCACTGGA
ACCCACAATTTGGTCTCTTGGTGTCAACATCAGCCAGGCAGAGCCCCCAAATTATTAATTTATGAC
TTCAATAAGAGGCCCTCAGGGGTCCCTGATCGCTTCTCTGGCTCCGGGTCTGGCGGCACGGCCTCC
CTGACCATCACTGGACTCCAGGATGACGATGACGCCGAATATTTTTGTTGGGCGTATGAAGCTTTT
GGCGGAGGGACCAAGTTGACCGTTCTTGGTCAGCCCAAGGCTGCCCCCTCGGTCACTCTGTTCCCG
CCCTCCTCTGAGGAGCTTCAAGCCAACAAGGCCACACTGGTGTGTCTCATAAGTGACTTCTACCCG
GGAGCCGTGACAGTGGCTTGGAAAGCAGATAGCAGCCCCGTCAAGGCGGGAGTGGAGACCACCA
CACCCTCCAAACAAAGCAACAACAAGTACGCGGCCAGCAGCTATCTGAGCCTGACGCCTGAGCAG
TGGAAGTCCCACAGAAGCTACAGCTGCCAGGTCACGCATGAAGGGAGCACCGTGGAGAAGACAG
TGGCCCCTACAGAATGTTCA
>N49P7YTE
HEAVY CHAIN
SEQ ID NO: 197

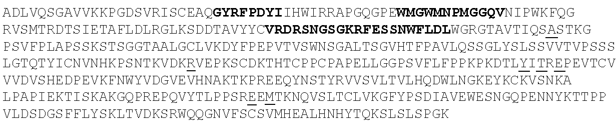

SEQ ID NO: 198
GCGGACTTGGTGCAGTCTGGGGCTGTGGTGAAGAAGCCTGGGGACTCAGTGAGGATCTCCTGTGA
GGCTCAAGGATATAGATTTCCTGACTACATCATTCACTGGATTCGACGGGCCCCTGGACAAGGCCC
TGAATGGATGGGATGGATGAATCCAATGGGCGGACAAGTAAATATTCCATGGAAATTTCAGGGTA
GGGTCTCCATGACCCGGGACACGTCCATCGAAACAGCATTTCTGGACTTAAGAGGACTAAAGTCT
GACGACACGGCCGTCTATTATTGCGTGAGAGATCGCAGTAATGGATCGGGAAAGCGATTCGAGTC
CTCCAATTGGTTCCTCGATCTGTGGGGCCGTGGGACTGCGGTCACAATTCAATCAGCCTCCACCAA
GGGCCCATCGGTCTTCCCCCTGGCACCCTCCTCCAAGAGCACCTCTGGGGGCACAGCGGCCCTGG
GCTGCCTGGTCAAGGACTACTTCCCCGAACCGGTGACGGTGTCGTGGAACTCAGGCGCCCTGACC
AGCGGCGTGCACACCTTCCCGGCTGTCCTACAGTCCTCAGGACTCTACTCCCTCAGCAGCGTGGTG
ACCGTGCCCTCCAGCAGCTTGGGCACCCAGACCTACATCTGCAACGTGAATCACAAGCCCAGCAA
CACCAAGGTGGACAAGAGAGTTGAGCCCAAATCTTGTGACAAAACTCACACATGCCCACCGTGCC
CAGCACCTGAACTCCTGGGGGGACCGTCAGTCTTCCTCTTCCCCCCAAAACCCAAGGACACCCTCT
ATATCACCCGGGAGCCTGAGGTCACATGCGTGGTGGTGGACGTGAGCCACGAAGACCCTGAGGTC
AAGTTCAACTGGTACGTGGACGGCGTGGAGGTGCATAATGCCAAGACAAAGCCGCGGGAGGAGC
AGTACAACAGCACGTACCGGGTGGTCAGCGTCCTCACCGTCCTGCACCAGGACTGGCTGAATGGC
AAGGAGTACAAGTGCAAGGTCTCCAACAAAGCCCTCCCAGCCCCCATCGAGAAAACCATCTCCAA
AGCCAAAGGGCAGCCCCGAGAACCACAGGTGTACACCCTGCCCCCATCCCGGGAGGAGATGACC
AAGAACCAGGTCAGCCTGACCTGCCTGGTCAAAGGCTTCTATCCCAGCGACATCGCCGTGGAGTG
GGAGAGCAATGGGCAGCCGGAGAACAACTACAAGACCACGCCTCCCGTGCTGGACTCCGACGGC
TCCTTCTTCCTCTACAGCAAGCTCACCGTGGACAAGAGCAGGTGGCAGCAGGGGAACGTCTTCTC
ATGCTCCGTGATGCATGAGGCTCTGCACAACCACTACACGCAGAAGAGCCTCTCCCTGTCTCCGGG
TAAA
LIGHT CHAIN
SEQ ID NO: 199

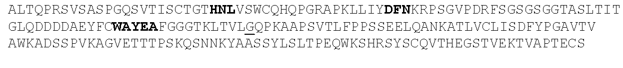

SEQ ID NO: 200
GCCCTGACTCAGCCTCGCTCAGTGTCCGCATCTCCTGGGCAGTCCGTCACCATTTCCTGCACTGGA
ACCCACAATTTGGTCTCTTGGTGTCAACATCAGCCAGGCAGAGCCCCCAAATTATTAATTTATGAC
TTCAATAAGAGGCCCTCAGGGGTCCCTGATCGCTTCTCTGGCTCCGGGTCTGGCGGCACGGCCTCC
CTGACCATCACTGGACTCCAGGATGACGATGACGCCGAATATTTTTGTTGGGCGTATGAAGCTTTT
GGCGGAGGGACCAAGTTGACCGTTCTTGGTCAGCCCAAGGCTGCCCCCTCGGTCACTCTGTTCCCG
CCCTCCTCTGAGGAGCTTCAAGCCAACAAGGCCACACTGGTGTGTCTCATAAGTGACTTCTACCCG
GGAGCCGTGACAGTGGCTTGGAAAGCAGATAGCAGCCCCGTCAAGGCGGGAGTGGAGACCACCA
CACCCTCCAAACAAAGCAACAACAAGTACGCGGCCAGCAGCTATCTGAGCCTGACGCCTGAGCAG
TGGAAGTCCCACAGAAGCTACAGCTGCCAGGTCACGCATGAAGGGAGCACCGTGGAGAAGACAG
TGGCCCCTACAGAATGTTCA
>N49P7L6
HEAVY CHAIN
SEQ ID NO: 201

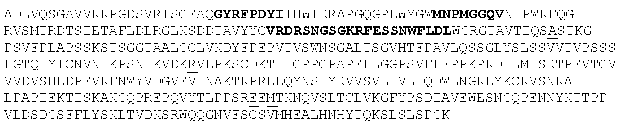

SEQ ID NO: 202
GCGGACTTGGTGCAGTCTGGGGCTGTGGTGAAGAAGCCTGGGGACTCAGTGAGGATCTCCTGTGA
GGCTCAAGGATATAGATTTCCTGACTACATCATTCACTGGATTCGACGGGCCCCTGGACAAGGCCC
TGAATGGATGGGATGGATGAATCCAATGGGCGGACAAGTAAATATTCCATGGAAATTTCAGGGTA
GGGTCTCCATGACCCGGGACACGTCCATCGAAACAGCATTTCTGGACTTAAGAGGACTAAAGTCT
GACGACACGGCCGTCTATTATTGCGTGAGAGATCGCAGTAATGGATCGGGAAAGCGATTCGAGTC
CTCCAATTGGTTCCTCGATCTGTGGGGCCGTGGGACTGCGGTCACAATTCAATCAGCCTCCACCAA
GGGCCCATCGGTCTTCCCCCTGGCACCCTCCTCCAAGAGCACCTCTGGGGGCACAGCGGCCCTGG
GCTGCCTGGTCAAGGACTACTTCCCCGAACCGGTGACGGTGTCGTGGAACTCAGGCGCCCTGACC
AGCGGCGTGCACACCTTCCCGGCTGTCCTACAGTCCTCAGGACTCTACTCCCTCAGCAGCGTGGTG
ACCGTGCCCTCCAGCAGCTTGGGCACCCAGACCTACATCTGCAACGTGAATCACAAGCCCAGCAA
CACCAAGGTGGACAAGAGAGTTGAGCCCAAATCTTGTGACAAAACTCACACATGCCCACCGTGCC
CAGCACCTGAACTCCTGGGGGGACCGTCAGTCTTCCTCTTCCCCCCAAAACCCAAGGACACCCTCA
TGATCTCCCGGACCCCTGAGGTCACATGCGTGGTGGTGGACGTGAGCCACGAAGACCCTGAGGTC
AAGTTCAACTGGTACGTGGACGGCGTGGAGGTGCATAATGCCAAGACAAAGCCGCGGGAGGAGC
AGTACAACAGCACGTACCGGGTGGTCAGCGTCCTCACCGTCCTGCACCAGGACTGGCTGAATGGC
AAGGAGTACAAGTGCAAGGTCTCCAACAAAGCCCTCCCAGCCCCCATCGAGAAAACCATCTCCAA
AGCCAAAGGGCAGCCCCGAGAACCACAGGTGTACACCCTGCCCCCATCCCGGGAGGAGATGACC
AAGAACCAGGTCAGCCTGACCTGCCTGGTCAAAGGCTTCTATCCCAGCGACATCGCCGTGGAGTG
GGAGAGCAATGGGCAGCCGGAGAACAACTACAAGACCACGCCTCCCGTGCTGGACTCCGACGGC
TCCTTCTTCCTCTACAGCAAGCTCACCGTGGACAAGAGCAGGTGGCAGCAGGGGAACGTCITTCTC
ATGCTCCGTGATGCATGAGGCTCTGCACAACCACTACACGCAGAAGAGCCTCTCCCTGTCTCCGGG
TAAA
LIGHT CHAIN
SEQ ID NO: 203

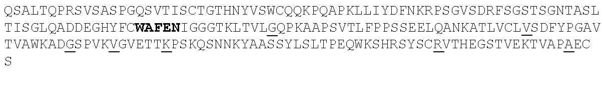

SEQ ID NO: 204
CAGTCTGCCCTAACTCAGCCTCGCTCAGTGTCCGCATCTCCTGGTCAGTCAGTCACCATCTCCTGC
ACTGGAACACACAATTATGTGTCCTGGTGTCAACAGAAACCGGGCCAAGCCCCCAAATTATTAAT
TTACGATTTCAATAAACGGCCCTCAGGGGTCTCTGATCGCTTCTCTGGCTCCACGTCTGGCAACAC
GGCCTCCCTGACCATCTCTGGACTCCAGGCTGACGATGAGGGTCATTATTTTTGTTGGGCGTTTGA
AAATATCGGCGGAGGGACCAAGCTGACCGTCCTGGGTCAGCCCAAGGCTGCCCCCTCGGTCACTC
TGTTCCCACCCTCCTCTGAGGAGCTTCAAGCCAACAAGGCCACACTGGTGTGTCTCGTAAGTGACT
TCTACCCGGGAGCCGTGACAGTGGCCTGGAAGGCAGATGGCAGCCCCGTCAAGGTGGGAGTGGA
GACCACCAAACCCTCCAAACAAAGCAACAACAAGTATGCGGCCAGCAGCTACCTGAGCCTGACGC
CCGAGCAGTGGAAGTCCCACAGAAGCTACAGCTGCCGGGTCACGCATGAAGGGAGCACCGTGGA
GAAGACAGTGGCCCCTGCAGAATGCTCT
>N49P7L11
HEAVY CHAIN
SEQ ID NO: 205

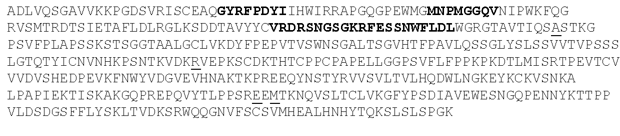

SEQ ID NO: 206
GCGGACTTGGTGCAGTCTGGGGCTGTGGTGAAGAAGCCTGGGGACTCAGTGAGGATCTCCTGTGA
GGCTCAAGGATATAGATTTCCTGACTACATCATTCACTGGATTCGACGGGCCCCTGGACAAGGCCC
TGAATGGATGGGATGGATGAATCCAATGGGCGGACAAGTAAATATTCCATGGAAATTTCAGGGTA
GGGTCTCCATGACCCGGGACACGTCCATCGAAACAGCATTTCTGGACTTAAGAGGACTAAAGTCT
GACGACACGGCCGTCTATTATTGCGTGAGAGATCGCAGTAATGGATCGGGAAAGCGATTCGAGTC
CTCCAATTGGTTCCTCGATCTGTGGGGCCGTGGGACTGCGGTCACAATTCAATCAGCCTCCACCAA
GGGCCCATCGGTCTTCCCCCTGGCACCCTCCTCCAAGAGCACCTCTGGGGGCACAGCGGCCCTGG
GCTGCCTGGTCAAGGACTACTTCCCCGAACCGGTGACGGTGTCGTGGAACTCAGGCGCCCTGACC
AGCGGCGTGCACACCTTCCCGGCTGTCCTACAGTCCTCAGGACTCTACTCCCTCAGCAGCGTGGTG
ACCGTGCCCTCCAGCAGCTTGGGCACCCAGACCTACATCTGCAACGTGAATCACAAGCCCAGCAA
CACCAAGGTGGACAAGAGAGTTGAGCCCAAATCTTGTGACAAAACTCACACATGCCCACCGTGCC
CAGCACCTGAACTCCTGGGGGGACCGTCAGTCTTCCTCTTCCCCCCAAAACCCAAGGACACCCTCA
TGATCTCCCGGACCCCTGAGGTCACATGCGTGGTGGTGGACGTGAGCCACGAAGACCCTGAGGTC
AAGTTCAACTGGTACGTGGACGGCGTGGAGGTGCATAATGCCAAGACAAAGCCGCGGGAGGAGC
AGTACAACAGCACGTACCGGGTGGTCAGCGTCCTCACCGTCCTGCACCAGGACTGGCTGAATGGC
AAGGAGTACAAGTGCAAGGTCTCCAACAAAGCCCTCCCAGCCCCCATCGAGAAAACCATCTCCAA
AGCCAAAGGGCAGCCCCGAGAACCACAGGTGTACACCCTGCCCCCATCCCGGGAGGAGATGACC
AAGAACCAGGTCAGCCTGACCTGCCTGGTCAAAGGCTTCTATCCCAGCGACATCGCCGTGGAGTG
GGAGAGCAATGGGCAGCCGGAGAACAACTACAAGACCACGCCTCCCGTGCTGGACTCCGACGGC
TCCTTCTTCCTCTACAGCAAGCTCACCGTGGACAAGAGCAGGTGGCAGCAGGGGAACGTCTTCTC
ATGCTCCGTGATGCATGAGGCTCTGCACAACCACTACACGCAGAAGAGCCTCTCCCTGTCTCCGGG
TAAA
LIGHT CHAIN
SEQ ID NO: 207

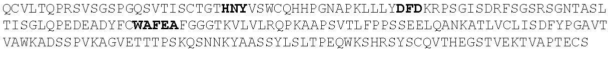

SEQ ID NO: 208
CAGTGTGTCTTGACTCAGCCTCGCTCAGTGTCCGGATCTCCTGGACAATCAGTCACCATCTCCTGC
ACTGGAACTCACAATTATGTCTCCTGGTGTCAACACCACCCAGGCAACGCCCCCAAATTATTACTT
TATGATTTCGACAAGCGGCCCTCAGGAATCTCTGATCGCTTCTCTGGCTCTAGGTCTGGCAACACG
GCCTCCCTGACCATCTCTGGCCTCCAGCCTGAGGATGAGGCCGATTACTTTTGTTGGGCCTTTGAA
GCCTTTGGCGGAGGGACCAAGGTGCTCGTCCTTCGTCAGCCCAAGGCTGCCCCCTCGGTCACTCTG
TTCCCGCCCTCCTCTGAGGAGCTTCAAGCCAACAAGGCCACACTGGTGTGTCTCATAAGTGACTTC
TACCCGGGAGCCGTGACAGTGGCTTGGAAAGCAGATAGCAGCCCCGTCAAGGCGGGAGTGGAGA
CCACCACACCCTCCAAACAAAGCAACAACAAGTACGCGGCCAGCAGCTATCTGAGCCTGACGCCT
GAGCAGTGGAAGTCCCACAGAAGCTACAGCTGCCAGGTCACGCATGAAGGGAGCACCGTGGAGA
AGACAGTGGCCCCTACAGAATGTTCA
>N49P7.1L9
HEAVY CHAIN
SEQ ID NO: 209

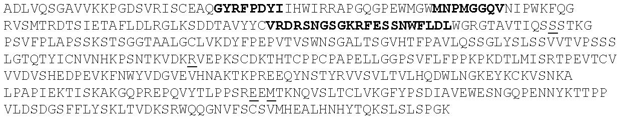

SEQ ID NO: 210
GCGGACTTGGTGCAGTCTGGGGCTGTGGTGAAGAAGCCTGGGGACTCAGTGAGGATCTCCTGTGA
GGCTCAAGGATATAGATTTCCTGACTACATCATTCACTGGATTCGACGGGCCCCTGGACAAGGCCC
TGAATGGATGGGATGGATGAATCCAATGGGCGGACAAGTAAATATTCCATGGAAATTTCAGGGTA
GGGTCTCCATGACCCGGGACACGTCCATCGAAACAGCATTTCTGGACTTAAGAGGACTAAAGTCT
GACGACACGGCCGTCTATTATTGCGTGAGAGATCGCAGTAATGGATCGGGAAAGCGATTCGAGTC
CTCCAATTGGTTCCTCGATCTGTGGGGCCGTGGGACTGCGGTCACAATTCAATCATCCTCCACCAA
GGGCCCATCGGTCTTCCCCCTGGCACCCTCCTCCAAGAGCACCTCTGGGGGCACAGCGGCCCTGG
GCTGCCTGGTCAAGGACTACTTCCCCGAACCGGTGACGGTGTCGTGGAACTCAGGCGCCCTGACC
AGCGGCGTGCACACCTTCCCGGCTGTCCTACAGTCCTCAGGACTCTACTCCCTCAGCAGCGTGGTG
ACCGTGCCCTCCAGCAGCTTGGGCACCCAGACCTACATCTGCAACGTGAATCACAAGCCCAGCAA
CACCAAGGTGGACAAGAGAGTTGAGCCCAAATCTTGTGACAAAACTCACACATGCCCACCGTGCC
CAGCACCTGAACTCCTGGGGGGACCGTCAGTCTTCCTCTTCCCCCCAAAACCCAAGGACACCCTCA
TGATCTCCCGGACCCCTGAGGTCACATGCGTGGTGGTGGACGTGAGCCACGAAGACCCTGAGGTC
AAGTTCAACTGGTACGTGGACGGCGTGGAGGTGCATAATGCCAAGACAAAGCCGCGGGAGGAGC
AGTACAACAGCACGTACCGGGTGGTCAGCGTCCTCACCGTCCTGCACCAGGACTGGCTGAATGGC
AAGGAGTACAAGTGCAAGGTCTCCAACAAAGCCCTCCCAGCCCCCATCGAGAAAACCATCTCCAA
AGCCAAAGGGCAGCCCCGAGAACCACAGGTGTACACCCTGCCCCCATCCCGGGAGGAGATGACC
AAGAACCAGGTCAGCCTGACCTGCCTGGTCAAAGGCTTCTATCCCAGCGACATCGCCGTGGAGTG
GGAGAGCAATGGGCAGCCGGAGAACAACTACAAGACCACGCCTCCCGTGCTGGACTCCGACGGC
TCCTTCTTCCTCTACAGCAAGCTCACCGTGGACAAGAGCAGGTGGCAGCAGGGGAACGTCTTCTC
ATGCTCCGTGATGCATGAGGCTCTGCACAACCACTACACGCAGAAGAGCCTCTCCCTGTCTCCGGG
TAAA
LIGHT CHAIN
SEQ ID NO: 211

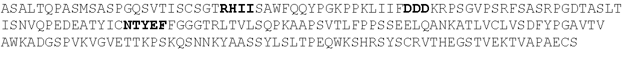

SEQ ID NO: 212
GCATCTGCCCTGACTCAGCCTGCCTCCATGTCTGCGTCCCCTGGACAGTCGGTAACCATCTCGTGC
TCTGGAACCAGACACATAATCTCTGCTTGGTTCCAACAATATCCAGGCAAACCACCCAAACTCATA
ATTTTTGACGACGATAAGCGTCCCTCTGGAGTTCCTAGTCGCTTCTCTGCCTCCAGGCCTGGCGAC
ACGGCCTCCCTGACAATCTCTAATGTTCAACCTGAGGACGAGGCGACGTACATTTGCAATACATAT
GAATTCTTTGGCGGAGGGACCAGATTGACCGTCCTAAGTCAGCCCAAGGCTGCCCCCTCGGTCACT
CTGTTCCCACCCTCCTCTGAGGAGCTTCAAGCCAACAAGGCCACACTGGTGTGTCTCGTAAGTGAC
TTCTACCCGGGAGCCGTGACAGTGGCCTGGAAGGCAGATGGCAGCCCCGTCAAGGTGGGAGTGGA
GACCACCAAACCCTCCAAACAAAGCAACAACAAGTATGCGGCCAGCAGCTACCTGAGCCTGACGC
CCGAGCAGTGGAAGTCCCACAGAAGCTACAGCTGCCGGGTCACGCATGAAGGGAGCACCGTGGA
GAAGACAGTGGCCCCTGCAGAATGCTCT
>N49P7.1L19
HEAVY CHAIN
SEQ ID NO: 213

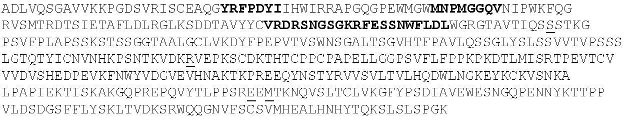

SEQ ID NO: 214
GCGGACTTGGTGCAGTCTGGGGCTGTGGTGAAGAAGCCTGGGGACTCAGTGAGGATCTCCTGTGA
GGCTCAAGGATATAGATTTCCTGACTACATCATTCACTGGATTCGACGGGCCCCTGGACAAGGCCC
TGAATGGATGGGATGGATGAATCCAATGGGCGGACAAGTAAATATTCCATGGAAATTTCAGGGTA
GGGTCTCCATGACCCGGGACACGTCCATCGAAACAGCATTTCTGGACTTAAGAGGACTAAAGTCT
GACGACACGGCCGTCTATTATTGCGTGAGAGATCGCAGTAATGGATCGGGAAAGCGATTCGAGTC
CTCCAATTGGTTCCTCGATCTGTGGGGCCGTGGGACTGCGGTCACAATTCAATCATCCTCCACCAA
GGGCCCATCGGTCTTCCCCCTGGCACCCTCCTCCAAGAGCACCTCTGGGGGCACAGCGGCCCTGG
GCTGCCTGGTCAAGGACTACTTCCCCGAACCGGTGACGGTGTCGTGGAACTCAGGCGCCCTGACC
AGCGGCGTGCACACCTTCCCGGCTGTCCTACAGTCCTCAGGACTCTACTCCCTCAGCAGCGTGGTG
ACCGTGCCCTCCAGCAGCTTGGGCACCCAGACCTACATCTGCAACGTGAATCACAAGCCCAGCAA
CACCAAGGTGGACAAGAGAGTTGAGCCCAAATCTTGTGACAAAACTCACACATGCCCACCGTGCC
CAGCACCTGAACTCCTGGGGGGACCGTCAGTCTTCCTCTTCCCCCCAAAACCCAAGGACACCCTCA
TGATCTCCCGGACCCCTGAGGTCACATGCGTGGTGGTGGACGTGAGCCACGAAGACCCTGAGGTC
AAGTTCAACTGGTACGTGGACGGCGTGGAGGTGCATAATGCCAAGACAAAGCCGCGGGAGGAGC
AGTACAACAGCACGTACCGGGTGGTCAGCGTCCTCACCGTCCTGCACCAGGACTGGCTGAATGGC
AAGGAGTACAAGTGCAAGGTCTCCAACAAAGCCCTCCCAGCCCCCATCGAGAAAACCATCTCCAA
AGCCAAAGGGCAGCCCCGAGAACCACAGGTGTACACCCTGCCCCCATCCCGGGAGGAGATGACC
AAGAACCAGGTCAGCCTGACCTGCCTGGTCAAAGGCTTCTATCCCAGCGACATCGCCGTGGAGTG
GGAGAGCAATGGGCAGCCGGAGAACAACTACAAGACCACGCCTCCCGTGCTGGACTCCGACGGC
TCCTTCTTCCTCTACAGCAAGCTCACCGTGGACAAGAGCAGGTGGCAGCAGGGGAACGTCTTCTC
ATGCTCCGTGATGCATGAGGCTCTGCACAACCACTACACGCAGAAGAGCCTCTCCCTGTCTCCGGG
TAAA
LIGHT CHAIN
SEQ ID NO: 215

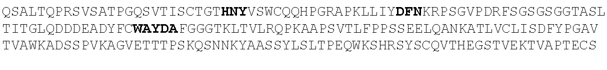

SEQ ID NO: 216
CAGTCTGCCCTGACTCAGCCTCGCTCAGTGTCCGCAACTCCTGGACAGTCAGTCACCATCTCCTGC
ACTGGAACCCACAATTATGTCTCTTGGTGTCAACAACATCCAGGCAGAGCCCCCAAATTACTAATT
TATGACTTCAATAAGAGGCCCTCAGGGGTCCCGGATCGCTTCTCTGGCTCCGGATCTGGCGGCACG
GCCTCCCTAACCATCACTGGACTCCAGGATGACGATGAAGCGGACTATTTTTGTTGGGCCTATGAT
GCTTTTGGCGGAGGGACCAAGTTGACCGTCCTGCGTCAGCCCAAGGCTGCCCCCTCGGTCACTCTG
TTCCCGCCCTCCTCTGAGGAGCTTCAAGCCAACAAGGCCACACTGGTGTGTCTCATAAGTGACTTC
TACCCGGGAGCCGTGACAGTGGCTTGGAAAGCAGATAGCAGCCCCGTCAAGGCGGGAGTGGAGA
CCACCACACCCTCCAAACAAAGCAACAACAAGTACGCGGCCAGCAGCTATCTGAGCCTGACGCCT
GAGCAGTGGAAGTCCCACAGAAGCTACAGCTGCCAGGTCACGCATGAAGGGAGCACCGTGGAGA
AGACAGTGGCCCCTACAGAATGTTCA
>R49P7
HEAVY CHAIN
SEQ ID NO: 217

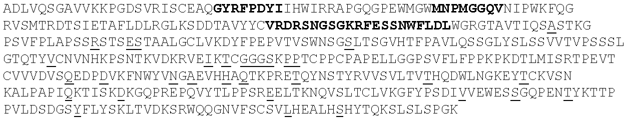

SEQ ID NO: 218
GCGGACTTGGTGCAGTCTGGGGCTGTGGTGAAGAAGCCTGGGGACTCAGTGAGGATCTCCTGTGA
GGCTCAAGGATATAGATTTCCTGACTACATCATTCACTGGATTCGACGGGCCCCTGGACAAGGCCC
TGAATGGATGGGATGGATGAATCCAATGGGCGGACAAGTAAATATTCCATGGAAATTTCAGGGTA
GGGTCTCCATGACCCGGGACACGTCCATCGAAACAGCATTTCTGGACTTAAGAGGACTAAAGTCT
GACGACACGGCCGTCTATTATTGCGTGAGAGATCGCAGTAATGGATCGGGAAAGCGATTCGAGTC
CTCCAATTGGTTCCTCGATCTGTGGGGCCGTGGGACTGCGGTCACAATTCAATCAGCCTCGACCAA
GGGCCCATCGGTCTTCCCCCTGGCGCCCTCCTCCAGGAGCACCTCCGAGAGCACAGCGGCCCTGG
GCTGCCTGGTCAAGGACTACTTCCCTGAACCCGTGACCGTGTCGTGGAACTCAGGCTCCCTGACCA
GCGGCGTGCACACCTTCCCGGCTGTCCTACAGTCCTCAGGGCTCTACTCCCTCAGCAGCGTGGTGA
CCGTGCCCTCCAGCAGCTTGGGCACCCAGACCTACGTCTGCAACGTAAACCACAAGCCCAGCAAC
ACCAAGGTGGACAAGAGAGTTGAGATAAAAACATGTGGTGGTGGCAGCAAACCTCCCACGTGCCC
ACCGTGCCCAGCACCTGAACTCCTGGGGGGACCGTCAGTCTTCCTCTTCCCCCCAAAACCCAAGGA
CACCCTCATGATCTCCCGGACCCCTGAGGTCACATGCGTGGTGGTAGACGTGAGCCAGGAAGACC
CCGATGTCAAGTTCAACTGGTACGTAAATGGCGCGGAGGTGCATCATGCCCAGACGAAGCCACGG
GAGACGCAGTACAACAGCACATATCGTGTGGTCAGCGTCCTCACCGTCACGCACCAGGACTGGCT
GAACGGCAAGGAGTACACGTGCAAGGTCTCCAACAAAGCCCTCCCGGCCCCCATCCAGAAAACCA
TCTCCAAAGACAAAGGGCAGCCCCGAGAGCCTCAGGTGTACACCCTGCCCCCGTCCCGGGAGGAG
CTGACCAAGAACCAGGTCAGCCTGACCTGCCTGGTCAAAGGCTTCTACCCCAGTGACATCGTCGT
GGAGTGGGAGAGCAGCGGGCAGCCGGAGAACACCTACAAGACCACCCCGCCCGTGCTGGACTCC
GACGGCTCCTACTTCCTCTACAGCAAGCTCACCGTGGACAAGAGCAGGTGGCAGCAGGGGAACGT
CTTCTCATGCTCCGTGCTGCATGAGGCTCTGCACAGCCACTACACGCAGAAGAGCCTCTCCCTGTC
CCCGGGTAAA
LIGHT CHAIN
SEQ ID NO: 219

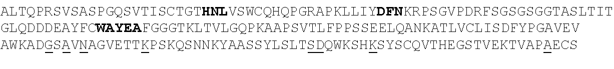

SEQ ID NO: 220
GCCCTGACTCAGCCTCGCTCAGTGTCCGCATCTCCTGGGCAGTCCGTCACCATTTCCTGCACTGGA
ACCCACAATTTGGTCTCTTGGTGTCAACATCAGCCAGGCAGAGCCCCCAAATTATTAATTTATGAC
TTCAATAAGAGGCCCTCAGGGGTCCCTGATCGCTTCTCTGGCTCCGGGTCTGGCGGCACGGCCTCC
CTGACCATCACTGGACTCCAGGATGACGATGACGCCGAATATTTTTGTTGGGCGTATGAAGCTTTT
GGCGGAGGGACCAAGTTGACCGTTCTTGGTCAGCCCAAGGCTGCCCCCTCGGTCACTCTGTTCCCG
CCCTCGAGCGAGGAGCTTCAAGCCAACAAGGCCACACTAGTGTGTCTGATCAGTGACTTCTACCC
GGGAGCCGTGGAAGTGGCCTGGAAGGCAGATGGCAGCGCTGTCAACGCGGGAGTGGAGACCACC
AAACCCTCCAAACAGAGCAACAACAAGTACGCGGCCAGCAGCTACCTGAGCCTGACGTCCGACCA
GTGGAAGTCCCACAAGAGCTACAGCTGCCAGGTCACGCACGAAGGGAGCACCGTGGAGAAGACA
GTGGCCCCTGCAGAATGTTCA
>N49P7.2
HEAVY CHAIN
SEQ ID NO: 221

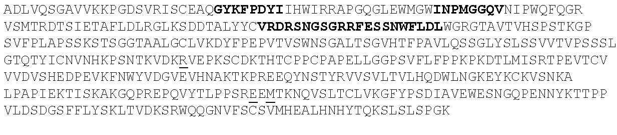

SEQ ID NO: 222
GCGGACTTGGTGCAGTCTGGGGCTGTGGTGAAGAAGCCTGGGGACTCAGTGAGGATCTCCTGTGA
GGCTCAAGGATACAAATTTCCTGACTACATCATTCACTGGATTCGACGCGCCCCTGGACAAGGCCT
TGAGTGGATGGGGTGGATTAATCCAATGGGCGGACAAGTAAACATTCCATGGCAGTTTCAGGGCA
GGGTCTCCATGACCCGGGACACGTCCATCGAAACAGCATTTCTGGACTTAAGAGGACTAAAGTCT
GACGACACGGCCCTCTATTATTGCGTGAGAGATCGAAGTAATGGATCGGGAAGGCGATTCGAGTC
CTCCAATTGGTTCCTCGATCTGTGGGGCCGCGGCACTGCGGTCACTGTTCATTCACCCTCCACCAA
GGGCCCATCGGTCTTCCCCCTGGCACCCTCCTCCAAGAGCACCTCTGGGGGCACAGCGGCCCTGG
GCTGCCTGGTCAAGGACTACTTCCCCGAACCGGTGACGGTGTCGTGGAACTCAGGCGCCCTGACC
AGCGGCGTGCACACCTTCCCGGCTGTCCTACAGTCCTCAGGACTCTACTCCCTCAGCAGCGTGGTG
ACCGTGCCCTCCAGCAGCTTGGGCACCCAGACCTACATCTGCAACGTGAATCACAAGCCCAGCAA
CACCAAGGTGGACAAGAGAGTTGAGCCCAAATCTTGTGACAAAACTCACACATGCCCACCGTGCC
CAGCACCTGAACTCCTGGGGGGACCGTCAGTCTTCCTCTTCCCCCCAAAACCCAAGGACACCCTCA
TGATCTCCCGGACCCCTGAGGTCACATGCGTGGTGGTGGACGTGAGCCACGAAGACCCTGAGGTC
AAGTTCAACTGGTACGTGGACGGCGTGGAGGTGCATAATGCCAAGACAAAGCCGCGGGAGGAGC
AGTACAACAGCACGTACCGGGTGGTCAGCGTCCTCACCGTCCTGCACCAGGACTGGCTGAATGGC
AAGGAGTACAAGTGCAAGGTCTCCAACAAAGCCCTCCCAGCCCCCATCGAGAAAACCATCTCCAA
AGCCAAAGGGCAGCCCCGAGAACCACAGGTGTACACCCTGCCCCCATCCCGGGAGGAGATGACC
AAGAACCAGGTCAGCCTGACCTGCCTGGTCAAAGGCTTCTATCCCAGCGACATCGCCGTGGAGTG
GGAGAGCAATGGGCAGCCGGAGAACAACTACAAGACCACGCCTCCCGTGCTGGACTCCGACGGC
TCCTTCTTCCTCTACAGCAAGCTCACCGTGGACAAGAGCAGGTGGCAGCAGGGGAACGTCTTCTC
ATGCTCCGTGATGCATGAGGCTCTGCACAACCACTACACGCAGAAGAGCCTCTCCCTGTCTCCGGG
TAAA
LIGHT CHAIN
SEQ ID NO: 223

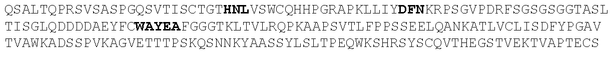

SEQ ID NO: 224
CAGTCTGCCCTGACTCAGCCTCGCTCAGTGTCCGCATCTCCTGGGCAGTCCGTCACCATTTCCTGC
ACTGGAACCCACAATTTGGTCTCTTGGTGTCAACATCACCCAGGCAGAGCCCCCAAATTATTAATT
TATGACTTCAATAAGAGGCCCTCAGGGGTCCCTGATCGCTTCTCTGGCTCCGGGTCTGGCGGCACG
GCCTCCCTGACCATCAGTGGACTCCAGGATGACGATGACGCCGAATATTTTTGTTGGGCGTATGAA
GCTTTTGGCGGAGGGACCAAGTTGACCGTACTTCGTCAGCCCAAGGCTGCCCCCTCGGTCACTCTG
TTCCCGCCCTCCTCTGAGGAGCTTCAAGCCAACAAGGCCACACTGGTGTGTCTCATAAGTGACTTC
TACCCGGGAGCCGTGACAGTGGCTTGGAAAGCAGATAGCAGCCCCGTCAAGGCGGGAGTGGAGA
CCACCACACCCTCCAAACAAAGCAACAACAAGTACGCGGCCAGCAGCTATCTGAGCCTGACGCCT
GAGCAGTGGAAGTCCCACAGAAGCTACAGCTGCCAGGTCACGCATGAAGGGAGCACCGTGGAGA
AGACAGTGGCCCCTACAGAATGTTCA
>N49P11
HEAVY CHAIN
SEQ ID NO: 225

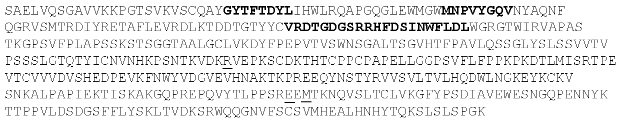

SEQ ID NO: 226
TCGGCGGAATTGGTGCAATCTGGGGCTGTGGTGAAGAAGCCTGGGACCTCCGTGAAGGTCTCITTG
TCAGGCTTATGGATACACTTTTACCGACTACCTTATTCATTGGCTTCGACAGGCCCCTGGACAAGG
ACTTGAATGGATGGGATGGATGAATCCTGTTTATGGACAAGTAAATTATGCCCAAAACTTTCAGG
GCAGGGTCTCCATGACCAGGGACATTTACAGGGAAACAGCATTTCTAGAGGTGCGCGACCTGAAG
ACTGACGACACAGGCACTTATTATTGTGTGAGAGACACAGGCGACGGTTCGCGGAGACACTTTGA
CTCCATCAATTGGTTTCTCGATCTTTGGGGCCGCGGGACATGGATAAGGGTCGCCCCAGCCTCCAC
CAAGGGCCCATCGGTCTTCCCCCTGGCACCCTCCTCCAAGAGCACCTCTGGGGGCACAGCGGCCCT
GGGCTGCCTGGTCAAGGACTACTTCCCCGAACCGGTGACGGTGTCGTGGAACTCAGGCGCCCTGA
CCAGCGGCGTGCACACCTTCCCGGCTGTCCTACAGTCCTCAGGACTCTACTCCCTCAGCAGCGTGG
TGACCGTGCCCTCCAGCAGCTTGGGCACCCAGACCTACATCTGCAACGTGAATCACAAGCCCAGC
AACACCAAGGTGGACAAGAGAGTTGAGCCCAAATCTTGTGACAAAACTCACACATGCCCACCGTG
CCCAGCACCTGAACTCCTGGGGGGACCGTCAGTCTTCCTCTTCCCCCCAAAACCCAAGGACACCCT
CATGATCTCCCGGACCCCTGAGGTCACATGCGTGGTGGTGGACGTGAGCCACGAAGACCCTGAGG
TCAAGTTCAACTGGTACGTGGACGGCGTGGAGGTGCATAATGCCAAGACAAAGCCGCGGGAGGA
GCAGTACAACAGCACGTACCGGGTGGTCAGCGTCCTCACCGTCCTGCACCAGGACTGGCTGAATG
GCAAGGAGTACAAGTGCAAGGTCTCCAACAAAGCCCTCCCAGCCCCCATCGAGAAAACCATCTCC
AAAGCCAAAGGGCAGCCCCGAGAACCACAGGTGTACACCCTGCCCCCATCCCGGGAGGAGATGA
CCAAGAACCAGGTCAGCCTGACCTGCCTGGTCAAAGGCTTCTATCCCAGCGACATCGCCGTGGAG
TGGGAGAGCAATGGGCAGCCGGAGAACAACTACAAGACCACGCCTCCCGTGCTGGACTCCGACG
GCTCCTTCTTCCTCTACAGCAAGCTCACCGTGGACAAGAGCAGGTGGCAGCAGGGGAACGTCTTC
TCATGCTCCGTGATGCATGAGGCTCTGCACAACCACTACACGCAGAAGAGCCTCTCCCTGTCTCCG
GGTAAA
LIGHT CHAIN
SEQ ID NO: 227

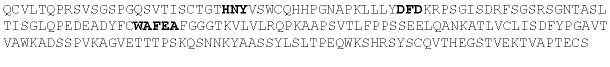

SEQ ID NO: 228
CAGTGTGTCTTGACTCAGCCTCGCTCAGTGTCCGGATCTCCTGGACAATCAGTCACCATCTCCTGC
ACTGGAACTCACAATTATGTCTCCTGGTGTCAACACCACCCAGGCAACGCCCCCAAATTATTACTT
TATGATTTCGACAAGCGGCCCTCAGGAATCTCTGATCGCTTCTCTGGCTCTAGGTCTGGCAACACG
GCCTCCCTGACCATCTCTGGCCTCCAGCCTGAGGATGAGGCCGATTACTTTTGTTGGGCCTTTGAA
GCCTTTGGCGGAGGGACCAAGGTGCTCGTCCTTCGTCAGCCCAAGGCTGCCCCCTCGGTCACTCTG
TTCCCGCCCTCCTCTGAGGAGCTTCAAGCCAACAAGGCCACACTGGTGTGTCTCATAAGTGACTTC
TACCCGGGAGCCGTGACAGTGGCTTGGAAAGCAGATAGCAGCCCCGTCAAGGCGGGAGTGGAGA
CCACCACACCCTCCAAACAAAGCAACAACAAGTACGCGGCCAGCAGCTATCTGAGCCTGACGCCT
GAGCAGTGGAAGTCCCACAGAAGCTACAGCTGCCAGGTCACGCATGAAGGGAGCACCGTGGAGA
AGACAGTGGCCCCTACAGAATGTTCA
>N49P18
HEAVY CHAIN
SEQ ID NO: 229

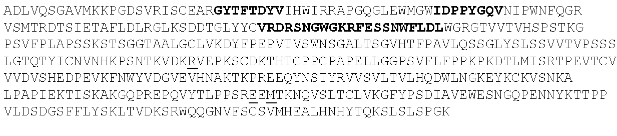

SEQ ID NO: 230
GCGGACTTGGTGCAGTCTGGGGCTGTGATGAAGAAGCCTGGGGACTCAGTGAGAATCTCCTGTGA
GGCTCGAGGATACACATTCACTGACTACGTCATTCACTGGATTCGACGCGCCCCTGGACAAGGCCT
TGAATGGATGGGGTGGATTGATCCACCTTATGGACAAGTAAATATTCCATGGAATTTTCAGGGCA
GGGTCTCCATGACCCGGGACACGTCCATCGAAACAGCATTTCTGGACTTAAGAGGTCTAAAGTCT
GACGACACGGGCCTCTATTATTGCGTGAGAGATCGAAGTAATGGATGGGGAAAGCGATTCGAGTC
CTCCAATTGGTTCCTCGATCTGTGGGGCCGCGGCACTGTGGTCACTGTTCACTCACCCTCCACCAA
GGGCCCATCGGTCTTCCCCCTGGCACCCTCCTCCAAGAGCACCTCTGGGGGCACAGCGGCCCTGG
GCTGCCTGGTCAAGGACTACTTCCCCGAACCGGTGACGGTGTCGTGGAACTCAGGCGCCCTGACC
AGCGGCGTGCACACCTTCCCGGCTGTCCTACAGTCCTCAGGACTCTACTCCCTCAGCAGCGTGGTG
ACCGTGCCCTCCAGCAGCTTGGGCACCCAGACCTACATCTGCAACGTGAATCACAAGCCCAGCAA
CACCAAGGTGGACAAGAGAGTTGAGCCCAAATCTTGTGACAAAACTCACACATGCCCACCGTGCC
CAGCACCTGAACTCCTGGGGGGACCGTCAGTCTTCCTCTTCCCCCCAAAACCCAAGGACACCCTCA
TGATCTCCCGGACCCCTGAGGTCACATGCGTGGTGGTGGACGTGAGCCACGAAGACCCTGAGGTC
AAGTTCAACTGGTACGTGGACGGCGTGGAGGTGCATAATGCCAAGACAAAGCCGCGGGAGGAGC
AGTACAACAGCACGTACCGGGTGGTCAGCGTCCTCACCGTCCTGCACCAGGACTGGCTGAATGGC
AAGGAGTACAAGTGCAAGGTCTCCAACAAAGCCCTCCCAGCCCCCATCGAGAAAACCATCTCCAA
AGCCAAAGGGCAGCCCCGAGAACCACAGGTGTACACCCTGCCCCCATCCCGGGAGGAGATGACC
AAGAACCAGGTCAGCCTGACCTGCCTGGTCAAAGGCTTCTATCCCAGCGACATCGCCGTGGAGTG
GGAGAGCAATGGGCAGCCGGAGAACAACTACAAGACCACGCCTCCCGTGCTGGACTCCGACGGC
TCCTTCTTCCTCTACAGCAAGCTCACCGTGGACAAGAGCAGGTGGCAGCAGGGGAACGTCTTCTC
ATGCTCCGTGATGCATGAGGCTCTGCACAACCACTACACGCAGAAGAGCCTCTCCCTGTCTCCGGG
TAAA
LIGHT CHAIN
SEQ ID NO: 231

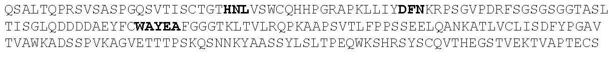

SEQ ID NO: 232
CAGTCTGCCCTGACTCAGCCTCGCTCAGTGTCCGCATCTCCTGGGCAGTCCGTCACCATTTCCTGC
ACTGGAACCCACAATTTGGTCTCTTGGTGTCAACATCACCCAGGCAGAGCCCCCAAGTTATTAATT
TATGACTTCAATAAGAGGCCCTCAGGGGTCCCTGATCGCTTCTCTGGCTCCGGGTCTGGCGGCACG
GCCTCCCTAACCATCAGTGGACTCCAGGATGACGATGACGCCGAATATTTTTGTTGGGCATATGAA
GCTTTCGGCGGAGGGACCAAGTTGACTGTACTTCGTCAGCCCAAGGCTGCCCCCTCGGTCACTCTG
TTCCCGCCCTCCTCTGAGGAGCTTCAAGCCAACAAGGCCACACTGGTGTGTCTCATAAGTGACTTC
TACCCGGGAGCCGTGACAGTGGCTTGGAAAGCAGATAGCAGCCCCGTCAAGGCGGGAGTGGAGA
CCACCACACCCTCCAAACAAAGCAACAACAAGTACGCGGCCAGCAGCTATCTGAGCCTGACGCCT
GAGCAGTGGAAGTCCCACAGAAGCTACAGCTGCCAGGTCACGCATGAAGGGAGCACCGTGGAGA
AGACAGTGGCCCCTACAGAATGTTCA
>N49P18.2
HEAVY CHAIN
SEQ ID NO: 233

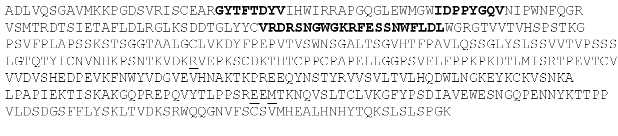

SEQ ID NO: 234
GCGGACTTGGTGCAGTCTGGGGCTGTGATGAAGAAGCCTGGGGACTCAGTGAGAATCTCCTGTGA
GGCTCGAGGATACACATTCACTGACTACGTCATTCACTGGATTCGACGCGCCCCTGGACAAGGCCT
TGAATGGATGGGGTGGATTGATCCACCTTATGGACAAGTAAATATTCCATGGAATTTTCAGGGCA
GGGTCTCCATGACCCGGGACACGTCCATCGAAACAGCATTTCTGGACTTAAGAGGTCTAAAGTCT
GACGACACGGGCCTCTATTATTGCGTGAGAGATCGAAGTAATGGATGGGGAAAGCGATTCGAGTC
CTCCAATTGGTTCCTCGATCTGTGGGGCCGCGGCACTGTGGTCACTGTTCACTCACCCTCCACCAA
GGGCCCATCGGTCTTCCCCCTGGCACCCTCCTCCAAGAGCACCTCTGGGGGCACAGCGGCCCTGG
GCTGCCTGGTCAAGGACTACTTCCCCGAACCGGTGACGGTGTCGTGGAACTCAGGCGCCCTGACC
AGCGGCGTGCACACCTTCCCGGCTGTCCTACAGTCCTCAGGACTCTACTCCCTCAGCAGCGTGGTG
ACCGTGCCCTCCAGCAGCTTGGGCACCCAGACCTACATCTGCAACGTGAATCACAAGCCCAGCAA
CACCAAGGTGGACAAGAGAGTTGAGCCCAAATCTTGTGACAAAACTCACACATGCCCACCGTGCC
CAGCACCTGAACTCCTGGGGGGACCGTCAGTCTTCCTCTTCCCCCCAAAACCCAAGGACACCCTCA
TGATCTCCCGGACCCCTGAGGTCACATGCGTGGTGGTGGACGTGAGCCACGAAGACCCTGAGGTC
AAGTTCAACTGGTACGTGGACGGCGTGGAGGTGCATAATGCCAAGACAAAGCCGCGGGAGGAGC
AGTACAACAGCACGTACCGGGTGGTCAGCGTCCTCACCGTCCTGCACCAGGACTGGCTGAATGGC
AAGGAGTACAAGTGCAAGGTCTCCAACAAAGCCCTCCCAGCCCCCATCGAGAAAACCATCTCCAA
AGCCAAAGGGCAGCCCCGAGAACCACAGGTGTACACCCTGCCCCCATCCCGGGAGGAGATGACC
AAGAACCAGGTCAGCCTGACCTGCCTGGTCAAAGGCTTCTATCCCAGCGACATCGCCGTGGAGTG
GGAGAGCAATGGGCAGCCGGAGAACAACTACAAGACCACGCCTCCCGTGCTGGACTCCGACGGC
TCCTTCTTCCTCTACAGCAAGCTCACCGTGGACAAGAGCAGGTGGCAGCAGGGGAACGTCTTCTC
ATGCTCCGTGATGCATGAGGCTCTGCACAACCACTACACGCAGAAGAGCCTCTCCCTGTCTCCGGG
TAAA
LIGHT CHAIN
SEQ ID NO: 235

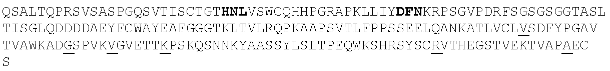

SEQ ID NO: 236
CAGTCTGCCCTGACTCAGCCTCGCTCAGTGTCCGCATCTCCTGGGCAGTCCGTCACCATTTCCTGC
ACTGGAACCCACAATTTGGTCTCTTGGTGTCAACATCACCCAGGCAGAGCCCCCAAGTTATTAATT
TATGACTTCAATAAGAGGCCCTCAGGGGTCCCTGATCGCTTCTCTGGCTCCGGGTCTGGCGGCACG
GCCTCCCTAACCATCAGTGGACTCCAGGATGACGATGACGCCGAATATTTTTGTTGGGCATATGAA
GCTTTCGGCGGAGGGACCAAGTTGACTGTACTTCGTCAGCCCAAGGCTGCCCCCTCGGTCACTCTG
TTCCCACCCTCCTCTGAGGAGCTTCAAGCCAACAAGGCCACACTGGTGTGTCTCGTAAGTGACTTC
TACCCGGGAGCCGTGACAGTGGCCTGGAAGGCAGATGGCAGCCCCGTCAAGGTGGGAGTGGAGA
CCACCAAACCCTCCAAACAAAGCAACAACAAGTATGCGGCCAGCAGCTACCTGAGCCTGACGCCC
GAGCAGTGGAAGTCCCACAGAAGCTACAGCTGCCGGGTCACGCATGAAGGGAGCACCGTGGAGA
AGACAGTGGCCCCTGCAGAATGCTCT
>N49P18.1
HEAVY CHAIN
SEQ ID NO: 237

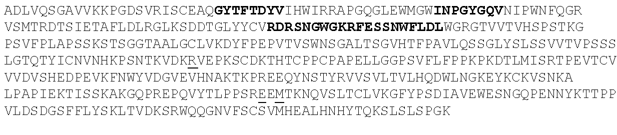

SEQ ID NO: 238
GCGGACTTGGTGCAGTCTGGGGCTGTGGTGAAGAAGCCTGGGGACTCAGTGAGAATCTCCTGTGA
GGCTCAAGGATACACATTCACTGACTACGTCATTCACTGGATTCGACGCGCCCCTGGACAAGGCCT
TGAATGGATGGGGTGGATTAATCCAGGTTATGGACAAGTAAATATTCCATGGAACTTTCAGGGCA
GGGTCTCCATGACCCGAGACACGTCCATCGAAACAGCATTTCTGGACTTAAGAGGTCTAAAGTCT
GACGACACGGGCCTCTATTATTGCGTGAGAGATCGAAGTAATGGATGGGGAAAGCGATTCGAGTC
CTCCAATTGGTTCCTCGATCTGTGGGGCCGCGGCACTGTGGTCACTGTTCACTCACCCTCCACCAA
GGGCCCATCGGTCTTCCCCCTGGCACCCTCCTCCAAGAGCACCTCTGGGGGCACAGCGGCCCTGG
GCTGCCTGGTCAAGGACTACTTCCCCGAACCGGTGACGGTGTCGTGGAACTCAGGCGCCCTGACC
AGCGGCGTGCACACCTTCCCGGCTGTCCTACAGTCCTCAGGACTCTACTCCCTCAGCAGCGTGGTG
ACCGTGCCCTCCAGCAGCTTGGGCACCCAGACCTACATCTGCAACGTGAATCACAAGCCCAGCAA
CACCAAGGTGGACAAGAGAGTTGAGCCCAAATCTTGTGACAAAACTCACACATGCCCACCGTGCC
CAGCACCTGAACTCCTGGGGGGACCGTCAGTCTTCCTCTTCCCCCCAAAACCCAAGGACACCCTCA
TGATCTCCCGGACCCCTGAGGTCACATGCGTGGTGGTGGACGTGAGCCACGAAGACCCTGAGGTC
AAGTTCAACTGGTACGTGGACGGCGTGGAGGTGCATAATGCCAAGACAAAGCCGCGGGAGGAGC
AGTACAACAGCACGTACCGGGTGGTCAGCGTCCTCACCGTCCTGCACCAGGACTGGCTGAATGGC
AAGGAGTACAAGTGCAAGGTCTCCAACAAAGCCCTCCCAGCCCCCATCGAGAAAACCATCTCCAA
AGCCAAAGGGCAGCCCCGAGAACCACAGGTGTACACCCTGCCCCCATCCCGGGAGGAGATGACC
AAGAACCAGGTCAGCCTGACCTGCCTGGTCAAAGGCTTCTATCCCAGCGACATCGCCGTGGAGTG
GGAGAGCAATGGGCAGCCGGAGAACAACTACAAGACCACGCCTCCCGTGCTGGACTCCGACGGC
TCCTTCTTCCTCTACAGCAAGCTCACCGTGGACAAGAGCAGGTGGCAGCAGGGGAACGTCTTCTC
ATGCTCCGTGATGCATGAGGCTCTGCACAACCACTACACGCAGAAGAGCCTCTCCCTGTCTCCGGG
TAAA
LIGHT CHAIN
SEQ ID NO: 239

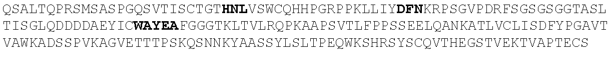

SEQ ID NO: 240
CAGTCTGCCCTGACTCAGCCTCGCTCAATGTCCGCATCTCCTGGGCAGTCCGTCACCATTTCCTGC
ACTGGAACCCACAATTTGGTCTCTTGGTGTCAACATCACCCAGGCAGACCCCCCAAATTATTAATT
TATGACTTCAATAAGAGGCCCTCAGGGGTCCCTGATCGCTTCTCTGGCTCCGGGTCTGGCGGCACG
GCCTCCCTGACCATCAGTGGACTCCAGGATGACGATGACGCCGAATACATTTGTTGGGCATATGA
AGCTTTCGGCGGAGGGACCAAGTTGACCGTACTTCGTCAGCCCAAGGCTGCCCCCTCGGTCACTCT
GTTCCCGCCCTCCTCTGAGGAGCTTCAAGCCAACAAGGCCACACTGGTGTGTCTCATAAGTGACTT
CTACCCGGGAGCCGTGACAGTGGCTTGGAAAGCAGATAGCAGCCCCGTCAAGGCGGGAGTGGAG
ACCACCACACCCTCCAAACAAAGCAACAACAAGTACGCGGCCAGCAGCTATCTGAGCCTGACGCC
TGAGCAGTGGAAGTCCCACAGAAGCTACAGCTGCCAGGTCACGCATGAAGGGAGCACCGTGGAG
AAGACAGTGGCCCCTACAGAATGTTCA
>N49P19
HEAVY CHAIN
SEQ ID NO: 241

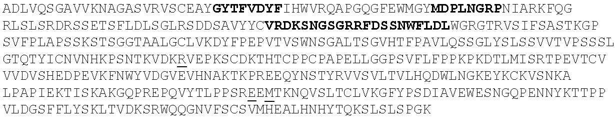

SEQ ID NO: 242
GCGGACTTGGTGCAGTCTGGGGCTGTGGTGAAAAATGCTGGGGCCTCAGTGAGGGTCTCCTGTGA
GGCTTATGGATACACATTCGTGGACTACTTCATTCATTGGGTCCGACAGGCCCCTGGACAAGGCTT
TGAATGGATGGGATACATGGATCCCTTGAACGGGCGCCCAAACATTGCGCGAAAATTTCAGGGCA
GGCTCTCCCTGAGTCGAGATAGGTCCAGCGAAACTTCATTTCTGGACTTAAGTGGACTGAGGTCTG
ACGACTCGGCCGTCTATTATTGTGTGAGAGACAAGAGTAATGGATCGGGCAGACGGTTITGACTCG
TCTAATTGGTTTCTCGATCTGTGGGGCCGTGGAACCCGGGTCAGTATTTTCTCAGCCTCCACCAAG
GGCCCATCGGTCTTCCCCCTGGCACCCTCCTCCAAGAGCACCTCTGGGGGCACAGCGGCCCTGGGC
TGCCTGGTCAAGGACTACTTCCCCGAACCGGTGACGGTGTCGTGGAACTCAGGCGCCCTGACCAG
CGGCGTGCACACCTTCCCGGCTGTCCTACAGTCCTCAGGACTCTACTCCCTCAGCAGCGTGGTGAC
CGTGCCCTCCAGCAGCTTGGGCACCCAGACCTACATCTGCAACGTGAATCACAAGCCCAGCAACA
CCAAGGTGGACAAGAGAGTTGAGCCCAAATCTTGTGACAAAACTCACACATGCCCACCGTGCCCA
GCACCTGAACTCCTGGGGGGACCGTCAGTCTTCCTCTTCCCCCCAAAACCCAAGGACACCCTCATG
ATCTCCCGGACCCCTGAGGTCACATGCGTGGTGGTGGACGTGAGCCACGAAGACCCTGAGGTCAA
GTTCAACTGGTACGTGGACGGCGTGGAGGTGCATAATGCCAAGACAAAGCCGCGGGAGGAGCAG
TACAACAGCACGTACCGGGTGGTCAGCGTCCTCACCGTCCTGCACCAGGACTGGCTGAATGGCAA
GGAGTACAAGTGCAAGGTCTCCAACAAAGCCCTCCCAGCCCCCATCGAGAAAACCATCTCCAAAG
CCAAAGGGCAGCCCCGAGAACCACAGGTGTACACCCTGCCCCCATCCCGGGAGGAGATGACCAA
GAACCAGGTCAGCCTGACCTGCCTGGTCAAAGGCTTCTATCCCAGCGACATCGCCGTGGAGTGGG
AGAGCAATGGGCAGCCGGAGAACAACTACAAGACCACGCCTCCCGTGCTGGACTCCGACGGCTCC
TTCTTCCTCTACAGCAAGCTCACCGTGGACAAGAGCAGGTGGCAGCAGGGGAACGTCTTCTCATG
CTCCGTGATGCATGAGGCTCTGCACAACCACTACACGCAGAAGAGCCTCTCCCTGTCTCCGGGTAA
A
LIGHT CHAIN
SEQ ID NO: 243

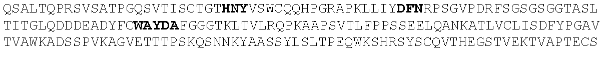

SEQ ID NO: 244
CAGTCTGCCCTGACTCAGCCTCGCTCAGTGTCCGCAACTCCTGGACAGTCAGTCACCATCTCCTGC
ACTGGAACCCACAATTATGTCTCTTGGTGTCAACAACATCCAGGCAGAGCCCCCAAATTACTAATT
TATGACTTCAATAAGAGGCCCTCAGGGGTCCCGGATCGCTTCTCTGGCTCCGGATCTGGCGGCACG
GCCTCCCTAACCATCACTGGACTCCAGGATGACGATGAAGCGGACTATTTTTGTTGGGCCTATGAT
GCTTTTGGCGGAGGGACCAAGTTGACCGTCCTGCGTCAGCCCAAGGCTGCCCCCTCGGTCACTCTG
TTCCCGCCCTCCTCTGAGGAGCTTCAAGCCAACAAGGCCACACTGGTGTGTCTCATAAGTGACTTC
TACCCGGGAGCCGTGACAGTGGCTTGGAAAGCAGATAGCAGCCCCGTCAAGGCGGGAGTGGAGA
CCACCACACCCTCCAAACAAAGCAACAACAAGTACGCGGCCAGCAGCTATCTGAGCCTGACGCCT
GAGCAGTGGAAGTCCCACAGAAGCTACAGCTGCCAGGTCACGCATGAAGGGAGCACCGTGGAGA
AGACAGTGGCCCCTACAGAATGTTCA
>N49P37
HEAVY CHAIN
SEQ ID NO: 245

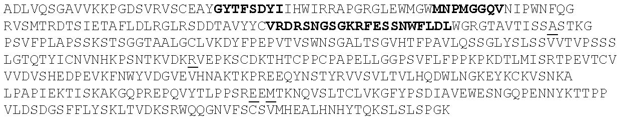

SEQ ID NO: 246
GCGGACTTGGTGCAGTCTGGGGCTGTGGTGAAGAAGCCTGGGGACTCAGTGAGGGTCTCCTGTGA
GGCTTATGGATACACATTCAGTGACTACATCATTCATTGGATTCGACGGGCCCCTGGACGAGGCCT
TGAATGGATGGGATGGATGAATCCGATGGGCGGACAAGTGAATATTCCGTGGAACTTTCAGGGGA
GAGTCTCCATGACCCGGGACACGTCCATCGAAACAGCATTTCTGGACTTAAGAGGACTGAGGTCT
GACGACACGGCCGTCTATTACTGTGTGAGAGATCGCAGCAATGGATCGGGCAAGCGATTTGAGTC
CTCCAATTGGTTCCTCGATCTGTGGGGCCGCGGGACCGCGGTCACTATTTCCTCAGCCTCCACCAA
GGGCCCATCGGTCTTCCCCCTGGCACCCTCCTCCAAGAGCACCTCTGGGGGCACAGCGGCCCTGG
GCTGCCTGGTCAAGGACTACTTCCCCGAACCGGTGACGGTGTCGTGGAACTCAGGCGCCCTGACC
AGCGGCGTGCACACCTTCCCGGCTGTCCTACAGTCCTCAGGACTCTACTCCCTCAGCAGCGTGGTG
ACCGTGCCCTCCAGCAGCTTGGGCACCCAGACCTACATCTGCAACGTGAATCACAAGCCCAGCAA
CACCAAGGTGGACAAGAGAGTTGAGCCCAAATCTTGTGACAAAACTCACACATGCCCACCGTGCC
CAGCACCTGAACTCCTGGGGGGACCGTCAGTCTTCCTCTTCCCCCCAAAACCCAAGGACACCCTCA
TGATCTCCCGGACCCCTGAGGTCACATGCGTGGTGGTGGACGTGAGCCACGAAGACCCTGAGGTC
AAGTTCAACTGGTACGTGGACGGCGTGGAGGTGCATAATGCCAAGACAAAGCCGCGGGAGGAGC
AGTACAACAGCACGTACCGGGTGGTCAGCGTCCTCACCGTCCTGCACCAGGACTGGCTGAATGGC
AAGGAGTACAAGTGCAAGGTCTCCAACAAAGCCCTCCCAGCCCCCATCGAGAAAACCATCTCCAA
AGCCAAAGGGCAGCCCCGAGAACCACAGGTGTACACCCTGCCCCCATCCCGGGAGGAGATGACC
AAGAACCAGGTCAGCCTGACCTGCCTGGTCAAAGGCTTCTATCCCAGCGACATCGCCGTGGAGTG
GGAGAGCAATGGGCAGCCGGAGAACAACTACAAGACCACGCCTCCCGTGCTGGACTCCGACGGC
TCCTTCTTCCTCTACAGCAAGCTCACCGTGGACAAGAGCAGGTGGCAGCAGGGGAACGTCTTCTC
ATGCTCCGTGATGCATGAGGCTCTGCACAACCACTACACGCAGAAGAGCCTCTCCCTGTCTCCGGG
TAAA
LIGHT CHAIN
SEQ ID NO: 247

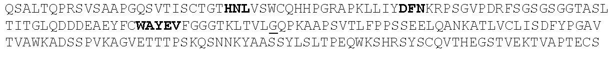

SEQ ID NO: 248
CAGTCTGCCCTGACTCAGCCTCGCTCAGTGTCCGCAGCTCCTGGACAGTCCGTCACCATTTCCTGC
ACTGGAACCCACAATTTGGTTTCTTGGTGTCAACATCACCCAGGCAGAGCCCCCAAGTTATTAATT
TATGACTTCAATAAGAGACCCTCAGGTGTCCCTGATCGTTTCTCTGGCTCCGGGTCTGGCGGCACG
GCCTCCCTAACCATCACTGGACTCCAGGATGACGATGAGGCTGAATATTTTTGTTGGGCGTATGAA
GTTTTTGGCGGAGGGACCAAGTTGACCGTGCTTGGTCAGCCCAAGGCTGCCCCCTCGGTCACTCTG
TTCCCGCCCTCCTCTGAGGAGCTTCAAGCCAACAAGGCCACACTGGTGTGTCTCATAAGTGACTTC
TACCCGGGAGCCGTGACAGTGGCTTGGAAAGCAGATAGCAGCCCCGTCAAGGCGGGAGTGGAGA
CCACCACACCCTCCAAACAAAGCAACAACAAGTACGCGGCCAGCAGCTATCTGAGCCTGACGCCT
GAGCAGTGGAAGTCCCACAGAAGCTACAGCTGCCAGGTCACGCATGAAGGGAGCACCGTGGAGA
AGACAGTGGCCCCTACAGAATGTTCA
>N49P38
HEAVY CHAIN
SEQ ID NO: 249

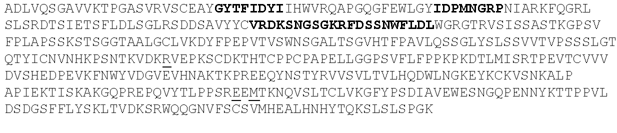

SEQ ID NO: 250
GCGGACTTGGTGCAGTCTGGGGCTGTGGTGAAGACGCCTGGGGCCTCAGTGAGGGTCTCCTGTGA
GGCTTATGGATACACATTCATTGACTACATCATTCATTGGGTCCGACAGGCCCCTGGACAAGGTTT
TGAATGGCTGGGATACATTGATCCTATGAACGGGCGCCCAAACATTGCGCGAAAATTTCAGGGCA
GGCTCTCCCTGAGCCGGGATACGTCCATCGAAACATCATTTCTGGACTTAAGTGGACTGAGGTCTG
ACGACTCGGCCGTCTATTATTGTGTGAGAGACAAGAGTAATGGATCGGGCAAACGATTTGACTCC
TCTAATTGGTTTCTCGATCTGTGGGGCCGTGGAACGCGGGTCAGCATTTCTTCAGCCTCCACCAAG
GGCCCATCGGTCTTCCCCCTGGCACCCTCCTCCAAGAGCACCTCTGGGGGCACAGCGGCCCTGGGC
TGCCTGGTCAAGGACTACTTCCCCGAACCGGTGACGGTGTCGTGGAACTCAGGCGCCCTGACCAG
CGGCGTGCACACCTTCCCGGCTGTCCTACAGTCCTCAGGACTCTACTCCCTCAGCAGCGTGGTGAC
CGTGCCCTCCAGCAGCTTGGGCACCCAGACCTACATCTGCAACGTGAATCACAAGCCCAGCAACA
CCAAGGTGGACAAGAGAGTTGAGCCCAAATCTTGTGACAAAACTCACACATGCCCACCGTGCCCA
GCACCTGAACTCCTGGGGGGACCGTCAGTCTTCCTCTTCCCCCCAAAACCCAAGGACACCCTCATG
ATCTCCCGGACCCCTGAGGTCACATGCGTGGTGGTGGACGTGAGCCACGAAGACCCTGAGGTCAA
GTTCAACTGGTACGTGGACGGCGTGGAGGTGCATAATGCCAAGACAAAGCCGCGGGAGGAGCAG
TACAACAGCACGTACCGGGTGGTCAGCGTCCTCACCGTCCTGCACCAGGACTGGCTGAATGGCAA
GGAGTACAAGTGCAAGGTCTCCAACAAAGCCCTCCCAGCCCCCATCGAGAAAACCATCTCCAAAG
CCAAAGGGCAGCCCCGAGAACCACAGGTGTACACCCTGCCCCCATCCCGGGAGGAGATGACCAA
GAACCAGGTCAGCCTGACCTGCCTGGTCAAAGGCTTCTATCCCAGCGACATCGCCGTGGAGTGGG
AGAGCAATGGGCAGCCGGAGAACAACTACAAGACCACGCCTCCCGTGCTGGACTCCGACGGCTCC
TTCTTCCTCTACAGCAAGCTCACCGTGGACAAGAGCAGGTGGCAGCAGGGGAACGTCTTCTCATG
CTCCGTGATGCATGAGGCTCTGCACAACCACTACACGCAGAAGAGCCTCTCCCTGTCTCCGGGTAA
A
LIGHT CHAIN
SEQ ID NO: 251

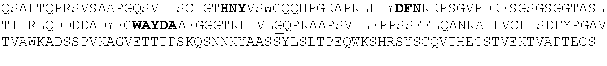

SEQ ID NO: 252
CAGTCTGCCCTGACTCAGCCTCGCTCAGTGTCCGCAGCTCCTGGACAGTCAGTCACCATCTCCTGC
ACTGGAACCCACAATTATGTCTCTTGGTGTCAACAACATCCAGGCAGAGCCCCCAAATTACTAATT
TATGACTTCAATAAGAGGCCCTCAGGGGTCCCGGATCGCTTCTCTGGCTCCGGATCTGGCGGCACG
GCCTCCCTAACCATCACTAGACTCCAGGATGACGATGACGCTGACTATTTTTGTTGGGCGTATGAT
GCTTTTGGCGGAGGGACCAAGTTGACCGTCCTGGGTCAGCCCAAGGCTGCCCCCTCGGTCACTCTG
TTCCCGCCCTCCTCTGAGGAGCTTCAAGCCAACAAGGCCACACTGGTGTGTCTCATAAGTGACTTC
TACCCGGGAGCCGTGACAGTGGCTTGGAAAGCAGATAGCAGCCCCGTCAAGGCGGGAGTGGAGA
CCACCACACCCTCCAAACAAAGCAACAACAAGTACGCGGCCAGCAGCTATCTGAGCCTGACGCCT
GAGCAGTGGAAGTCCCACAGAAGCTACAGCTGCCAGGTCACGCATGAAGGGAGCACCGTGGAGA
AGACAGTGGCCCCTACAGAATGTTCA
>N49P38.1
HEAVY CHAIN
SEQ ID NO: 253

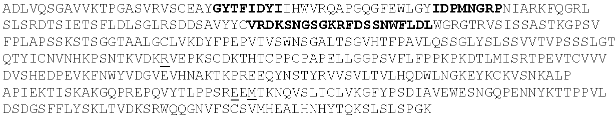

SEQ ID NO: 254
GCGGACTTGGTGCAGTCTGGGGCTGTGGTGAAGACGCCTGGGGCCTCAGTGAGGGTCTCCTGTGA
GGCTTATGGATACACATTCATTGACTACATCATTCATTGGGTCCGACAGGCCCCTGGACAAGGTTT
TGAATGGCTGGGATACATTGATCCTATGAACGGGCGCCCAAACATTGCGCGAAAATTTCAGGGCA
GGCTCTCCCTGAGCCGGGATACGTCCATCGAAACATCATTTCTGGACTTAAGTGGACTGAGGTCTG
ACGACTCGGCCGTCTATTATTGTGTGAGAGACAAGAGTAATGGATCGGGCAAACGATTTGACTCC
TCTAATTGGTTTCTCGATCTGTGGGGCCGTGGAACGCGGGTCAGCATTTCTTCAGCCTCCACCAAG
GGCCCATCGGTCTTCCCCCTGGCACCCTCCTCCAAGAGCACCTCTGGGGGCACAGCGGCCCTGGGC
TGCCTGGTCAAGGACTACTTCCCCGAACCGGTGACGGTGTCGTGGAACTCAGGCGCCCTGACCAG
CGGCGTGCACACCTTCCCGGCTGTCCTACAGTCCTCAGGACTCTACTCCCTCAGCAGCGTGGTGAC
CGTGCCCTCCAGCAGCTTGGGCACCCAGACCTACATCTGCAACGTGAATCACAAGCCCAGCAACA
CCAAGGTGGACAAGAGAGTTGAGCCCAAATCTTGTGACAAAACTCACACATGCCCACCGTGCCCA
GCACCTGAACTCCTGGGGGGACCGTCAGTCTTCCTCTTCCCCCCAAAACCCAAGGACACCCTCATG
ATCTCCCGGACCCCTGAGGTCACATGCGTGGTGGTGGACGTGAGCCACGAAGACCCTGAGGTCAA
GTTCAACTGGTACGTGGACGGCGTGGAGGTGCATAATGCCAAGACAAAGCCGCGGGAGGAGCAG
TACAACAGCACGTACCGGGTGGTCAGCGTCCTCACCGTCCTGCACCAGGACTGGCTGAATGGCAA
GGAGTACAAGTGCAAGGTCTCCAACAAAGCCCTCCCAGCCCCCATCGAGAAAACCATCTCCAAAG
CCAAAGGGCAGCCCCGAGAACCACAGGTGTACACCCTGCCCCCATCCCGGGAGGAGATGACCAA
GAACCAGGTCAGCCTGACCTGCCTGGTCAAAGGCTTCTATCCCAGCGACATCGCCGTGGAGTGGG
AGAGCAATGGGCAGCCGGAGAACAACTACAAGACCACGCCTCCCGTGCTGGACTCCGACGGCTCC
TTCTTCCTCTACAGCAAGCTCACCGTGGACAAGAGCAGGTGGCAGCAGGGGAACGTCTTCTCATG
CTCCGTGATGCATGAGGCTCTGCACAACCACTACACGCAGAAGAGCCTCTCCCTGTCTCCGGGTAA
A
LIGHT CHAIN
SEQ ID NO: 255

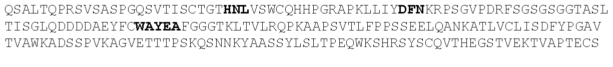

SEQ ID NO: 256
CAGTCTGCCCTGACTCAGCCTCGCTCAGTGTCCGCAGCTCCTGGACAGTCAGTCACCATCTCCTGC
ACTGGAACCCACAATTATGTCTCTTGGTGTCAACAACATCCAGGCAGAGCCCCCAAATTACTAATT
TATGACTTCAATAAGAGGCCCTCAGGGGTCCCGGATCGCTTCTCTGGCTCCGGATCTGGCGGCACG
GCCTCCCTAACCATCACTAGACTCCAGGATGACGATGACGCTGACTATTTTTGTTGGGCGTATGAT
GCTTTTGGCGGAGGGACCAAGTTGACCGTCCTGCGTCAGCCCAAGGCTGCCCCCTCGGTCACTCTG
TTCCCGCCCTCCTCTGAGGAGCTTCAAGCCAACAAGGCCACACTGGTGTGTCTCATAAGTGACTTC
TACCCGGGAGCCGTGACAGTGGCTTGGAAAGCAGATAGCAGCCCCGTCAAGGCGGGAGTGGAGA
CCACCACACCCTCCAAACAAAGCAACAACAAGTACGCGGCCAGCAGCTATCTGAGCCTGACGCCT
GAGCAGTGGAAGTCCCACAGAAGCTACAGCTGCCAGGTCACGCATGAAGGGAGCACCGTGGAGA
AGACAGTGGCCCCTACAGAATGTTCA
>N49P55
HEAVY CHAIN
SEQ ID NO: 257

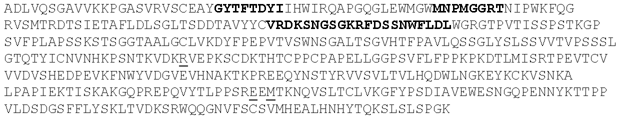

SEQ ID NO: 258
GCGGACTTGGTGCAGTCTGGGGCTGTGGTGAAGAAGCCTGGGGCCTCAGTGAGGGTCTCCTGTGA
GGCTTATGGATACACATTCACTGACTACATCATTCATTGGATTCGACAGGCCCCTGGACAAGGCCT
TGAATGGATGGGATGGATGAATCCTATGGGCGGGCGCACAAATATTCCGTGGAAATTTCAGGGCA
GGGTCTCCATGACCCGGGACACGTCCATCGAAACAGCATTTCTGGACTTAAGTGGACTAACGTCTG
ACGACACGGCCGTCTATTATTGCGTGAGAGACAAGAGTAATGGATCGGGCAAACGATTTGACTCC
TCTAATTGGTTCCTCGATCTGTGGGGCCGCGGAACCCCGGTCACTATTTCCTCACCCTCCACCAAG
GGCCCATCGGTCTTCCCCCTGGCACCCTCCTCCAAGAGCACCTCTGGGGGCACAGCGGCCCTGGGC
TGCCTGGTCAAGGACTACTTCCCCGAACCGGTGACGGTGTCGTGGAACTCAGGCGCCCTGACCAG
CGGCGTGCACACCTTCCCGGCTGTCCTACAGTCCTCAGGACTCTACTCCCTCAGCAGCGTGGTGAC
CGTGCCCTCCAGCAGCTTGGGCACCCAGACCTACATCTGCAACGTGAATCACAAGCCCAGCAACA
CCAAGGTGGACAAGAGAGTTGAGCCCAAATCTTGTGACAAAACTCACACATGCCCACCGTGCCCA
GCACCTGAACTCCTGGGGGGACCGTCAGTCTTCCTCTTCCCCCCAAAACCCAAGGACACCCTCATG
ATCTCCCGGACCCCTGAGGTCACATGCGTGGTGGTGGACGTGAGCCACGAAGACCCTGAGGTCAA
GTTCAACTGGTACGTGGACGGCGTGGAGGTGCATAATGCCAAGACAAAGCCGCGGGAGGAGCAG
TACAACAGCACGTACCGGGTGGTCAGCGTCCTCACCGTCCTGCACCAGGACTGGCTGAATGGCAA
GGAGTACAAGTGCAAGGTCTCCAACAAAGCCCTCCCAGCCCCCATCGAGAAAACCATCTCCAAAG
CCAAAGGGCAGCCCCGAGAACCACAGGTGTACACCCTGCCCCCATCCCGGGAGGAGATGACCAA
GAACCAGGTCAGCCTGACCTGCCTGGTCAAAGGCTTCTATCCCAGCGACATCGCCGTGGAGTGGG
AGAGCAATGGGCAGCCGGAGAACAACTACAAGACCACGCCTCCCGTGCTGGACTCCGACGGCTCC
TTCTTCCTCTACAGCAAGCTCACCGTGGACAAGAGCAGGTGGCAGCAGGGGAACGTCTTCTCATG
CTCCGTGATGCATGAGGCTCTGCACAACCACTACACGCAGAAGAGCCTCTCCCTGTCTCCGGGTAA
A
LIGHT CHAIN
SEQ ID NO: 259

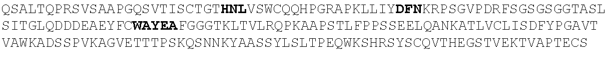

SEQ ID NO: 260
CAGTCTGCCCTGACTCAGCCTCGCTCAGTGTCCGCAGCTCCTGGACAGTCCGTCACCATTTCCTGC
ACTGGAACCCACAATTTGGTCTCTTGGTGTCAACAACACCCAGGCAGAGCCCCCAAATTATTAATT
TATGACTTCAATAAGAGGCCCTCAGGGGTCCCTGATCGCTTCTCTGGCTCCGGGTCTGGCGGCACG
GCCTCCCTAAGTATCACTGGACTCCAGGATGACGATGAAGCTGAATATTTTTGTTGGGCGTATGAA
GCTTTTGGCGGAGGGACCAAGTTGACCGTCCTTCGTCAGCCCAAGGCTGCCCCCTCGGTCACTCTG
TTCCCGCCCTCCTCTGAGGAGCTTCAAGCCAACAAGGCCACACTGGTGTGTCTCATAAGTGACTTC
TACCCGGGAGCCGTGACAGTGGCTTGGAAAGCAGATAGCAGCCCCGTCAAGGCGGGAGTGGAGA
CCACCACACCCTCCAAACAAAGCAACAACAAGTACGCGGCCAGCAGCTATCTGAGCCTGACGCCT
GAGCAGTGGAAGTCCCACAGAAGCTACAGCTGCCAGGTCACGCATGAAGGGAGCACCGTGGAGA
AGACAGTGGCCCCTACAGAATGTTCA
>N49P56
HEAVY CHAIN
SEQ ID NO: 261

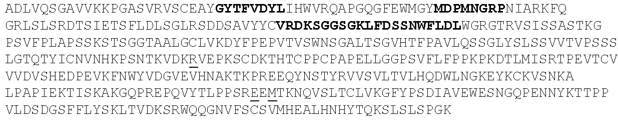

SEQ ID NO: 262
GCGGACTTGGTGCAGTCTGGGGCTGTGGTGAAGAAGCCTGGGGCCTCAGTGCGGGTCTCCTGTGA
GGCTTATGGATATACATTCGTTGACTACCTCATTCATTGGGTCCGACAGGCCCCCGGACAAGGTTT
TGAATGGATGGGATACATGGATCCTATGAACGGGCGCCCAAATATTGCGCGAAAATTTCAGGGCA
GGCTCTCCCTGAGCCGAGATACGTCCATCGAAACATCATTTCTGGACTTAAGTGGACTGAGGTCTG
ACGACTCGGCCGTCTATTATTGTGTGAGAGACAAGAGTGGTGGATCGGGCAAACTATTTGACTCCT
CTAATTGGTTTCTCGATCTGTGGGGCCGTGGAACCCGGGTCAGCATTTCTTCAGCCTCCACCAAGG
GCCCATCGGTCTTCCCCCTGGCACCCTCCTCCAAGAGCACCTCTGGGGGCACAGCGGCCCTGGGCT
GCCTGGTCAAGGACTACTTCCCCGAACCGGTGACGGTGTCGTGGAACTCAGGCGCCCTGACCAGC
GGCGTGCACACCTTCCCGGCTGTCCTACAGTCCTCAGGACTCTACTCCCTCAGCAGCGTGGTGACC
GTGCCCTCCAGCAGCTTGGGCACCCAGACCTACATCTGCAACGTGAATCACAAGCCCAGCAACAC
CAAGGTGGACAAGAGAGTTGAGCCCAAATCTTGTGACAAAACTCACACATGCCCACCGTGCCCAG
CACCTGAACTCCTGGGGGGACCGTCAGTCTTCCTCTTCCCCCCAAAACCCAAGGACACCCTCATGA
TCTCCCGGACCCCTGAGGTCACATGCGTGGTGGTGGACGTGAGCCACGAAGACCCTGAGGTCAAG
TTCAACTGGTACGTGGACGGCGTGGAGGTGCATAATGCCAAGACAAAGCCGCGGGAGGAGCAGT
ACAACAGCACGTACCGGGTGGTCAGCGTCCTCACCGTCCTGCACCAGGACTGGCTGAATGGCAAG
GAGTACAAGTGCAAGGTCTCCAACAAAGCCCTCCCAGCCCCCATCGAGAAAACCATCTCCAAAGC
CAAAGGGCAGCCCCGAGAACCACAGGTGTACACCCTGCCCCCATCCCGGGAGGAGATGACCAAG
AACCAGGTCAGCCTGACCTGCCTGGTCAAAGGCTTCTATCCCAGCGACATCGCCGTGGAGTGGGA
GAGCAATGGGCAGCCGGAGAACAACTACAAGACCACGCCTCCCGTGCTGGACTCCGACGGCTCCT
TCTTCCTCTACAGCAAGCTCACCGTGGACAAGAGCAGGTGGCAGCAGGGGAACGTCTITCTCATGC
TCCGTGATGCATGAGGCTCTGCACAACCACTACACGCAGAAGAGCCTCTCCCTGTCTCCGGGTAA
A
LIGHT CHAIN
SEQ ID NO: 263

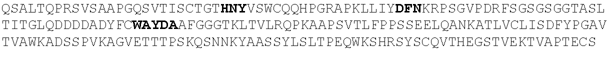

SEQ ID NO: 264
CAGTCTGCCCTGACTCAGCCTCGCTCAGTGTCCGCAGCTCCTGGACAGTCAGTCACCATCTCCTGC
ACCGGAACTCACAATTATGTCTCTTGGTGTCAACAACATCCAGGCAGAGCCCCCAAATTACTAATT
TATGACTTCAATAAGAGGCCCTCAGGGGTCCCGGATCGCTTCTCTGGCTCCGGATCTGGCGGCACG
GCCTCCCTAACCATCACTGGACTCCAGGATGACGATGACGCTGATTATTTTTGTTGGGCGTATGAT
GCTTTTGGCGGAGGGACCAAGTTGACCGTCCTGCGTCAGCCCAAGGCTGCCCCCTCGGTCACTCTG
TTCCCGCCCTCCTCTGAGGAGCTTCAAGCCAACAAGGCCACACTGGTGTGTCTCATAAGTGACTTC
TACCCGGGAGCCGTGACAGTGGCTTGGAAAGCAGATAGCAGCCCCGTCAAGGCGGGAGTGGAGA
CCACCACACCCTCCAAACAAAGCAACAACAAGTACGCGGCCAGCAGCTATCTGAGCCTGACGCCT
GAGCAGTGGAAGTCCCACAGAAGCTACAGCTGCCAGGTCACGCATGAAGGGAGCACCGTGGAGA
AGACAGTGGCCCCTACAGAATGTTCA
>N49P57
HEAVY CHAIN
SEQ ID NO: 265

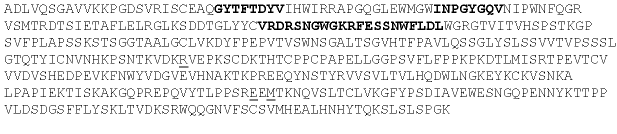

SEQ ID NO: 266
GCGGACTTGGTGCAGTCTGGGGCTGTGGTGAAGAAGCCTGGGGACTCAGTGAGAATCTCCTGTGA
GGCTCAAGGATACACATTCACTGACTACGTCATTCACTGGATTCGACGGGCCCCTGGACAAGGCC
TTGAATGGATGGGGTGGATTAATCCAGGTTATGGACAAGTAAATATTCCATGGAACTTTCAGGGC
AGGGTCTCCATGACCCGAGACACGTCCATCGAAACAGCTTTTCTGGAGTTAAGAGGTCTAAAGTCT
GACGACACGGGCCTCTATTATTGCGTGAGAGATCGAAGTAATGGATGGGGAAAGCGATTCGAGTC
CTCCAATTGGTTCCTCGATCTGTGGGGCCGCGGCACTGTGATTACTGTTCACTCACCCTCCACCAA
GGGCCCATCGGTCTTCCCCCTGGCACCCTCCTCCAAGAGCACCTCTGGGGGCACAGCGGCCCTGG
GCTGCCTGGTCAAGGACTACTTCCCCGAACCGGTGACGGTGTCGTGGAACTCAGGCGCCCTGACC
AGCGGCGTGCACACCTTCCCGGCTGTCCTACAGTCCTCAGGACTCTACTCCCTCAGCAGCGTGGTG
ACCGTGCCCTCCAGCAGCTTGGGCACCCAGACCTACATCTGCAACGTGAATCACAAGCCCAGCAA
CACCAAGGTGGACAAGAGAGTTGAGCCCAAATCTTGTGACAAAACTCACACATGCCCACCGTGCC
CAGCACCTGAACTCCTGGGGGGACCGTCAGTCTTCCTCTTCCCCCCAAAACCCAAGGACACCCTCA
TGATCTCCCGGACCCCTGAGGTCACATGCGTGGTGGTGGACGTGAGCCACGAAGACCCTGAGGTC
AAGTTCAACTGGTACGTGGACGGCGTGGAGGTGCATAATGCCAAGACAAAGCCGCGGGAGGAGC
AGTACAACAGCACGTACCGGGTGGTCAGCGTCCTCACCGTCCTGCACCAGGACTGGCTGAATGGC
AAGGAGTACAAGTGCAAGGTCTCCAACAAAGCCCTCCCAGCCCCCATCGAGAAAACCATCTCCAA
AGCCAAAGGGCAGCCCCGAGAACCACAGGTGTACACCCTGCCCCCATCCCGGGAGGAGATGACC
AAGAACCAGGTCAGCCTGACCTGCCTGGTCAAAGGCTTCTATCCCAGCGACATCGCCGTGGAGTG
GGAGAGCAATGGGCAGCCGGAGAACAACTACAAGACCACGCCTCCCGTGCTGGACTCCGACGGC
TCCTTCTTCCTCTACAGCAAGCTCACCGTGGACAAGAGCAGGTGGCAGCAGGGGAACGTCTTCTC
ATGCTCCGTGATGCATGAGGCTCTGCACAACCACTACACGCAGAAGAGCCTCTCCCTGTCTCCGGG
TAAA
LIGHT CHAIN
SEQ ID NO: 267

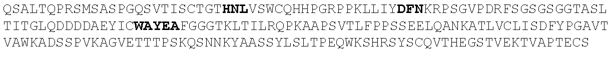

SEQ ID NO: 268
CAGTCTGCCCTGACTCAGCCTCGCTCAATGTCCGCATCTCCTGGGCAGTCCGTCACCATTTCCTGC
ACTGGAACCCACAATTTGGTCTCTTGGTGTCAACATCACCCAGGCAGACCCCCCAAATTATTAATT
TATGACTTCAATAAGAGGCCCTCAGGGGTCCCTGATCGCTTCTCTGGCTCCGGGTCTGGCGGCACG
GCCTCCCTGACCATCACTGGACTCCAGGATGACGATGACGCCGAATACATTTGTTGGGCATATGAA
GCTTTCGGCGGAGGGACCAAGTTGACCATACTTCGTCAGCCCAAGGCTGCCCCCTCGGTCACTCTG
TTCCCGCCCTCCTCTGAGGAGCTTCAAGCCAACAAGGCCACACTGGTGTGTCTCATAAGTGACTTC
TACCCGGGAGCCGTGACAGTGGCTTGGAAAGCAGATAGCAGCCCCGTCAAGGCGGGAGTGGAGA
CCACCACACCCTCCAAACAAAGCAACAACAAGTACGCGGCCAGCAGCTATCTGAGCCTGACGCCT
GAGCAGTGGAAGTCCCACAGAAGCTACAGCTGCCAGGTCACGCATGAAGGGAGCACCGTGGAGA
AGACAGTGGCCCCTACAGAATGTTCA
>N49P58
HEAVY CHAIN
SEQ ID NO: 269

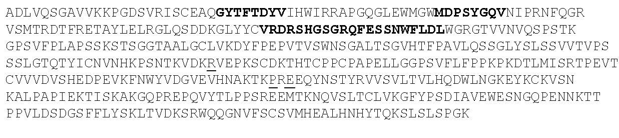

SEQ ID NO: 270
GCGGACTTGGTGCAGTCTGGGGCTGTGGTGAAGAAGCCTGGGGACTCAGTGAGAATCTCCTGTGA
GGCTCAGGGATATACATTCACCGACTACGTCATTCATTGGATTCGACGGGCCCCTGGACAAGGCCT
TGAATGGATGGGGTGGATGGATCCAAGTTATGGACAAGTCAATATTCCACGGAACTTTCAGGGCA
GGGTCTCCATGACCCGGGACACGTTCAGGGAAACAGCATATCTGGAATTAAGAGGTCTACAGTCT
GACGACAAGGGCCTCTATTATTGTGTGAGAGATCGAAGTCACGGATCGGGAAGGCAATTCGAGTC
CTCCAACTGGTTCCTCGATCTGTGGGGCCGCGGCACTGTGGTCAATGTTCAGTCACCCTCCACCAA
GGGCCCATCGGTCTTCCCCCTGGCACCCTCCTCCAAGAGCACCTCTGGGGGCACAGCGGCCCTGG
GCTGCCTGGTCAAGGACTACTTCCCCGAACCGGTGACGGTGTCGTGGAACTCAGGCGCCCTGACC
AGCGGCGTGCACACCTTCCCGGCTGTCCTACAGTCCTCAGGACTCTACTCCCTCAGCAGCGTGGTG
ACCGTGCCCTCCAGCAGCTTGGGCACCCAGACCTACATCTGCAACGTGAATCACAAGCCCAGCAA
CACCAAGGTGGACAAGAGAGTTGAGCCCAAATCTTGTGACAAAACTCACACATGCCCACCGTGCC
CAGCACCTGAACTCCTGGGGGGACCGTCAGTCTTCCTCTTCCCCCCAAAACCCAAGGACACCCTCA
TGATCTCCCGGACCCCTGAGGTCACATGCGTGGTGGTGGACGTGAGCCACGAAGACCCTGAGGTC
AAGTTCAACTGGTACGTGGACGGCGTGGAGGTGCATAATGCCAAGACAAAGCCGCGGGAGGAGC
AGTACAACAGCACGTACCGGGTGGTCAGCGTCCTCACCGTCCTGCACCAGGACTGGCTGAATGGC
AAGGAGTACAAGTGCAAGGTCTCCAACAAAGCCCTCCCAGCCCCCATCGAGAAAACCATCTCCAA
AGCCAAAGGGCAGCCCCGAGAACCACAGGTGTACACCCTGCCCCCATCCCGGGAGGAGATGACC
AAGAACCAGGTCAGCCTGACCTGCCTGGTCAAAGGCTTCTATCCCAGCGACATCGCCGTGGAGTG
GGAGAGCAATGGGCAGCCGGAGAACAACTACAAGACCACGCCTCCCGTGCTGGACTCCGACGGC
TCCTTCTTCCTCTACAGCAAGCTCACCGTGGACAAGAGCAGGTGGCAGCAGGGGAACGTCTTCTC
ATGCTCCGTGATGCATGAGGCTCTGCACAACCACTACACGCAGAAGAGCCTCTCCCTGTCTCCGGG
TAAA
LIGHT CHAIN
SEQ ID NO: 271

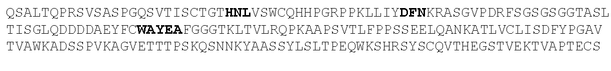

SEQ ID NO: 272
CAGTCTGCCCTGACTCAGCCTCGCTCAGTGTCCGCATCTCCTGGGCAGTCCGTCACCATTTCCTGC
ACTGGAACCCACAATTTGGTCTCTTGGTGTCAACATCACCCAGGCAGACCTCCCAAATTATTAATT
TATGACTTCAATAAGAGGGCTTCAGGGGTCCCTGATCGCTTCTCTGGCTCCGGGTCTGGCGGCACG
GCCTCCCTGACCATTAGTGGACTCCAGGATGACGATGACGCCGAATATTTTTGTTGGGCATATGAA
GCTTTCGGCGGAGGGACCAAGTTGACCGTACTTCGTCAGCCCAAGGCTGCCCCCTCGGTCACTCTG
TTCCCGCCCTCCTCTGAGGAGCTTCAAGCCAACAAGGCCACACTGGTGTGTCTCATAAGTGACTTC
TACCCGGGAGCCGTGACAGTGGCTTGGAAAGCAGATAGCAGCCCCGTCAAGGCGGGAGTGGAGA
CCACCACACCCTCCAAACAAAGCAACAACAAGTACGCGGCCAGCAGCTATCTGAGCCTGACGCCT
GAGCAGTGGAAGTCCCACAGAAGCTACAGCTGCCAGGTCACGCATGAAGGGAGCACCGTGGAGA
AGACAGTGGCCCCTACAGAATGTTCA
>N49P59
HEAVY CHAIN
SEQ ID NO: 273

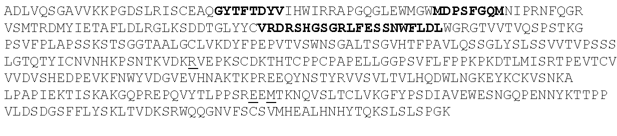

SEQ ID NO: 274
GCGGACTTGGTGCAGTCTGGGGCTGTGGTGAAGAAGCCTGGGGACTCACTGAGAATCTCCTGTGA
GGCTCAAGGATACACATTCACTGACTACGTCATTCACTGGATTCGACGGGCCCCTGGACAAGGCC
TTGAATGGATGGGATGGATGGATCCAAGTTTTGGACAAATGAACATTCCACGGAACTTTCAGGGC
AGGGTCTCCATGACCCGTGACATGTACATCGAAACAGCATTTCTGGACTTAAGAGGTCTAAAGTCT
GACGACACGGGCCTCTATTATTGCGTGAGAGATCGAAGTCATGGATCGGGAAGGCTATTCGAGTC
CTCCAATTGGTTCCTCGATCTGTGGGGCCGCGGCACTGTGGTCACTGTTCAGTCACCCTCCACCAA
GGGCCCATCGGTCTTCCCCCTGGCACCCTCCTCCAAGAGCACCTCTGGGGGCACAGCGGCCCTGG
GCTGCCTGGTCAAGGACTACTTCCCCGAACCGGTGACGGTGTCGTGGAACTCAGGCGCCCTGACC
AGCGGCGTGCACACCTTCCCGGCTGTCCTACAGTCCTCAGGACTCTACTCCCTCAGCAGCGTGGTG
ACCGTGCCCTCCAGCAGCTTGGGCACCCAGACCTACATCTGCAACGTGAATCACAAGCCCAGCAA
CACCAAGGTGGACAAGAGAGTTGAGCCCAAATCTTGTGACAAAACTCACACATGCCCACCGTGCC
CAGCACCTGAACTCCTGGGGGGACCGTCAGTCTTCCTCTTCCCCCCAAAACCCAAGGACACCCTCA
TGATCTCCCGGACCCCTGAGGTCACATGCGTGGTGGTGGACGTGAGCCACGAAGACCCTGAGGTC
AAGTTCAACTGGTACGTGGACGGCGTGGAGGTGCATAATGCCAAGACAAAGCCGCGGGAGGAGC
AGTACAACAGCACGTACCGGGTGGTCAGCGTCCTCACCGTCCTGCACCAGGACTGGCTGAATGGC
AAGGAGTACAAGTGCAAGGTCTCCAACAAAGCCCTCCCAGCCCCCATCGAGAAAACCATCTCCAA
AGCCAAAGGGCAGCCCCGAGAACCACAGGTGTACACCCTGCCCCCATCCCGGGAGGAGATGACC
AAGAACCAGGTCAGCCTGACCTGCCTGGTCAAAGGCTTCTATCCCAGCGACATCGCCGTGGAGTG
GGAGAGCAATGGGCAGCCGGAGAACAACTACAAGACCACGCCTCCCGTGCTGGACTCCGACGGC
TCCTTCTTCCTCTACAGCAAGCTCACCGTGGACAAGAGCAGGTGGCAGCAGGGGAACGTCTTCTC
ATGCTCCGTGATGCATGAGGCTCTGCACAACCACTACACGCAGAAGAGCCTCTCCCTGTCTCCGGG
TAAA
LIGHT CHAIN
SEQ ID NO: 275

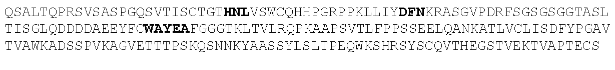

SEQ ID NO: 276
CAGTCTGCCCTGACTCAGCCTCGCTCAGTGTCCGCATCTCCTGGGCAGTCCGTCACCATTTCCTGC
ACTGGAACCCACAATTTGGTCTCTTGGTGTCAACATCACCCAGGCAGACCTCCCAAATTATTAATT
TATGACTTCAATAAGAGGGCTTCAGGGGTCCCTGATCGCTTCTCTGGCTCCGGGTCTGGCGGCACG
GCCTCCCTGACCATTAGTGGACTCCAGGATGACGATGACGCCGAATATTTTTGTTGGGCATATGAA
GCTTTCGGCGGAGGGACCAAGTTGACCGTACTTCGTCAGCCCAAGGCTGCCCCCTCGGTCACTCTG
TTCCCGCCCTCCTCTGAGGAGCTTCAAGCCAACAAGGCCACACTGGTGTGTCTCATAAGTGACTTC
TACCCGGGAGCCGTGACAGTGGCTTGGAAAGCAGATAGCAGCCCCGTCAAGGCGGGAGTGGAGA
CCACCACACCCTCCAAACAAAGCAACAACAAGTACGCGGCCAGCAGCTATCTGAGCCTGACGCCT
GAGCAGTGGAAGTCCCACAGAAGCTACAGCTGCCAGGTCACGCATGAAGGGAGCACCGTGGAGA
AGACAGTGGCCCCTACAGAATGTTCA
>N49P73
HEAVY CHAIN
SEQ ID NO: 277

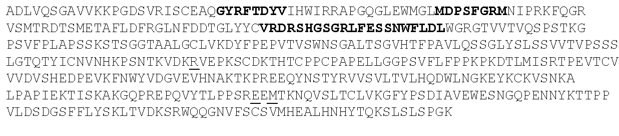

SEQ ID NO: 278
GCGGACTTGGTGCAGTCTGGGGCTGTGGTGAAGAAGCCTGGGGACTCAGTGAGAATCTCCTGTGA
GGCTCAAGGATACAGATTCACTGACTACGTCATTCATTGGATTCGACGGGCCCCTGGACAAGGCCT
TGAATGGATGGGGTTGATGGATCCAAGTTTTGGACGAATGAATATTCCACGGAAATTTCAGGGCA
GGGTCTCCATGACCCGGGACACGTCCATGGAAACAGCATTTCTGGACTTCAGAGGTCTAAATTTTG
ACGACACGGGCCTCTATTATTGCGTGAGAGATCGAAGTCATGGATCGGGAAGACTATTCGAGTCC
TCCAATTGGTTCCTCGATCTGTGGGGCCGCGGCACTGTGGTCACTGTTCAGTCACCCTCCACCAAG
GGCCCATCGGTCTTCCCCCTGGCACCCTCCTCCAAGAGCACCTCTGGGGGCACAGCGGCCCTGGGC
TGCCTGGTCAAGGACTACTTCCCCGAACCGGTGACGGTGTCGTGGAACTCAGGCGCCCTGACCAG
CGGCGTGCACACCTTCCCGGCTGTCCTACAGTCCTCAGGACTCTACTCCCTCAGCAGCGTGGTGAC
CGTGCCCTCCAGCAGCTTGGGCACCCAGACCTACATCTGCAACGTGAATCACAAGCCCAGCAACA
CCAAGGTGGACAAGAGAGTTGAGCCCAAATCTTGTGACAAAACTCACACATGCCCACCGTGCCCA
GCACCTGAACTCCTGGGGGGACCGTCAGTCTTCCTCTTCCCCCCAAAACCCAAGGACACCCTCATG
ATCTCCCGGACCCCTGAGGTCACATGCGTGGTGGTGGACGTGAGCCACGAAGACCCTGAGGTCAA
GTTCAACTGGTACGTGGACGGCGTGGAGGTGCATAATGCCAAGACAAAGCCGCGGGAGGAGCAG
TACAACAGCACGTACCGGGTGGTCAGCGTCCTCACCGTCCTGCACCAGGACTGGCTGAATGGCAA
GGAGTACAAGTGCAAGGTCTCCAACAAAGCCCTCCCAGCCCCCATCGAGAAAACCATCTCCAAAG
CCAAAGGGCAGCCCCGAGAACCACAGGTGTACACCCTGCCCCCATCCCGGGAGGAGATGACCAA
GAACCAGGTCAGCCTGACCTGCCTGGTCAAAGGCTTCTATCCCAGCGACATCGCCGTGGAGTGGG
AGAGCAATGGGCAGCCGGAGAACAACTACAAGACCACGCCTCCCGTGCTGGACTCCGACGGCTCC
TTCTTCCTCTACAGCAAGCTCACCGTGGACAAGAGCAGGTGGCAGCAGGGGAACGTCTTCTCATG
CTCCGTGATGCATGAGGCTCTGCACAACCACTACACGCAGAAGAGCCTCTCCCTGTCTCCGGGTAA
A
LIGHT CHAIN
SEQ ID NO: 279

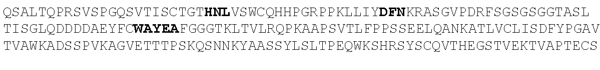

SEQ ID NO: 280
CAGTCTGCCCTGACTCAGCCTCGCTCAGTGTCCGCATCTCCTGGGCAGTCCGTCACCATTTCCTGC
ACTGGAACCCACAATTTGGTCTCTTGGTGTCAACATCACCCAGGCAGACCTCCCAAATTATTAATT
TATGACTTCAATAAGAGGGCATCAGGGGTCCCTGATCGCTTCTCTGGCTCCGGGTCTGGCGGCACG
GCCTCCCTGACCATCAGTGGACTCCAAGATGACGATGACGCCGAATATTTTTGTTGGGCATATGAA
GCTTTCGGCGGAGGGACCAAGTTGACCGTACTTCGTCAGCCCAAGGCTGCCCCCTCGGTCACTCTG
TTCCCGCCCTCCTCTGAGGAGCTTCAAGCCAACAAGGCCACACTGGTGTGTCTCATAAGTGACTTC
TACCCGGGAGCCGTGACAGTGGCTTGGAAAGCAGATAGCAGCCCCGTCAAGGCGGGAGTGGAGA
CCACCACACCCTCCAAACAAAGCAACAACAAGTACGCGGCCAGCAGCTATCTGAGCCTGACGCCT
GAGCAGTGGAAGTCCCACAGAAGCTACAGCTGCCAGGTCACGCATGAAGGGAGCACCGTGGAGA
AGACAGTGGCCCCTACAGAATGTTCA
>N49P74
HEAVY CHAIN
SEQ ID NO: 281

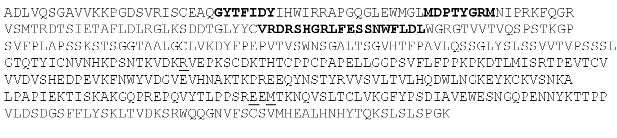

SEQ ID NO: 282
GCGGACTTGGTGCAGTCTGGGGCTGTGGTGAAGAAGCCTGGGGACTCAGTGAGAATTTCCTGTGA
GGCTCAAGGATACACATTCATTGACTACGTCATTCACTGGATTCGACGGGCCCCTGGACAAGGCCT
TGAATGGATGGGGTTGATGGATCCAACTTATGGACGAATGAATATTCCACGGAAGTTTCAGGGCA
GGGTCTCCATGACCCGGGACACGTCCATCGAAACAGCATTTCTGGACTTAAGAGGTCTAAAATCT
GACGACACGGGCCTCTATTATTGCGTGAGAGATCGAAGTCATGGATCGGGAAGGCTATTCGAGTC
CTCCAACTGGTTCCTGGATCTGTGGGGCCGCGGCACTGTGGTCACTGTTCAGTCACCCTCCACCAA
GGGCCCATCGGTCTTCCCCCTGGCACCCTCCTCCAAGAGCACCTCTGGGGGCACAGCGGCCCTGG
GCTGCCTGGTCAAGGACTACTTCCCCGAACCGGTGACGGTGTCGTGGAACTCAGGCGCCCTGACC
AGCGGCGTGCACACCTTCCCGGCTGTCCTACAGTCCTCAGGACTCTACTCCCTCAGCAGCGTGGTG
ACCGTGCCCTCCAGCAGCTTGGGCACCCAGACCTACATCTGCAACGTGAATCACAAGCCCAGCAA
CACCAAGGTGGACAAGAGAGTTGAGCCCAAATCTTGTGACAAAACTCACACATGCCCACCGTGCC
CAGCACCTGAACTCCTGGGGGGACCGTCAGTCTTCCTCTTCCCCCCAAAACCCAAGGACACCCTCA
TGATCTCCCGGACCCCTGAGGTCACATGCGTGGTGGTGGACGTGAGCCACGAAGACCCTGAGGTC
AAGTTCAACTGGTACGTGGACGGCGTGGAGGTGCATAATGCCAAGACAAAGCCGCGGGAGGAGC
AGTACAACAGCACGTACCGGGTGGTCAGCGTCCTCACCGTCCTGCACCAGGACTGGCTGAATGGC
AAGGAGTACAAGTGCAAGGTCTCCAACAAAGCCCTCCCAGCCCCCATCGAGAAAACCATCTCCAA
AGCCAAAGGGCAGCCCCGAGAACCACAGGTGTACACCCTGCCCCCATCCCGGGAGGAGATGACC
AAGAACCAGGTCAGCCTGACCTGCCTGGTCAAAGGCTTCTATCCCAGCGACATCGCCGTGGAGTG
GGAGAGCAATGGGCAGCCGGAGAACAACTACAAGACCACGCCTCCCGTGCTGGACTCCGACGGC
TCCTTCTTCCTCTACAGCAAGCTCACCGTGGACAAGAGCAGGTGGCAGCAGGGGAACGTCITTCTC
ATGCTCCGTGATGCATGAGGCTCTGCACAACCACTACACGCAGAAGAGCCTCTCCCTGTCTCCGGG
TAAA
LIGHT CHAIN
SEQ ID NO: 283

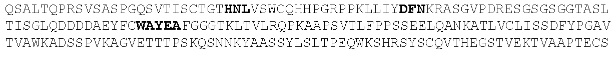

SEQ ID NO: 284
CAGTCTGCCCTGACTCAGCCTCGCTCAGTGTCCGCATCTCCTGGGCAGTCCGTCACCATTTCCTGC
ACTGGAACCCACAATTTGGTCTCTTGGTGTCAACATCACCCAGGCAGACCTCCCAAATTATTAATT
TATGACTTCAATAAGAGGGCTTCAGGGGTCCCTGATCGCTTCTCTGGCTCCGGGTCTGGCGGCACG
GCCTCCCTGACCATCAGTGGACTCCAAGATGACGATGACGCCGAATATTTTTGTTGGGCATATGAA
GCTTTCGGCGGAGGGACCAAGTTGACCGTACTTCGTCAGCCCAAGGCTGCCCCCTCGGTCACTCTG
TTCCCGCCCTCCTCTGAGGAGCTTCAAGCCAACAAGGCCACACTGGTGTGTCTCATAAGTGACTTC
TACCCGGGAGCCGTGACAGTGGCTTGGAAAGCAGATAGCAGCCCCGTCAAGGCGGGAGTGGAGA
CCACCACACCCTCCAAACAAAGCAACAACAAGTACGCGGCCAGCAGCTATCTGAGCCTGACGCCT
GAGCAGTGGAAGTCCCACAGAAGCTACAGCTGCCAGGTCACGCATGAAGGGAGCACCGTGGAGA
AGACAGTGGCCCCTACAGAATGTTCA
>N49P75
HEAVY CHAIN
SEQ ID NO: 285

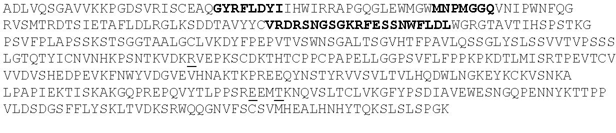

SEQ ID NO: 286
GCGGACTTGGTGCAGTCTGGGGCTGTGGTGAAGAAGCCTGGGGACTCAGTGAGGATCTCCTGTGA
GGCTCAAGGATACAGATTTCTTGACTACATCATTCACTGGATTCGACGAGCCCCTGGACAAGGCCT
TGAATGGATGGGATGGATGAATCCAATGGGCGGACAAGTAAACATTCCATGGAACTTTCAGGGTA
GGGTCTCCATGACCCGGGACACGTCCATCGAAACAGCATTTCTGGACTTAAGAGGACTAAAGTCT
GACGACACGGCCGTCTATTATTGCGTGAGAGATCGCAGTAATGGATCGGGAAAGCGATTCGAGTC
CTCCAATTGGTTCCTCGATCTGTGGGGCCGCGGGACTGCGGTCACTATTCATTCACCCTCCACCAA
GGGCCCATCGGTCTTCCCCCTGGCACCCTCCTCCAAGAGCACCTCTGGGGGCACAGCGGCCCTGG
GCTGCCTGGTCAAGGACTACTTCCCCGAACCGGTGACGGTGTCGTGGAACTCAGGCGCCCTGACC
AGCGGCGTGCACACCTTCCCGGCTGTCCTACAGTCCTCAGGACTCTACTCCCTCAGCAGCGTGGTG
ACCGTGCCCTCCAGCAGCTTGGGCACCCAGACCTACATCTGCAACGTGAATCACAAGCCCAGCAA
CACCAAGGTGGACAAGAGAGTTGAGCCCAAATCTTGTGACAAAACTCACACATGCCCACCGTGCC
CAGCACCTGAACTCCTGGGGGGACCGTCAGTCTTCCTCTTCCCCCCAAAACCCAAGGACACCCTCA
TGATCTCCCGGACCCCTGAGGTCACATGCGTGGTGGTGGACGTGAGCCACGAAGACCCTGAGGTC
AAGTTCAACTGGTACGTGGACGGCGTGGAGGTGCATAATGCCAAGACAAAGCCGCGGGAGGAGC
AGTACAACAGCACGTACCGGGTGGTCAGCGTCCTCACCGTCCTGCACCAGGACTGGCTGAATGGC
AAGGAGTACAAGTGCAAGGTCTCCAACAAAGCCCTCCCAGCCCCCATCGAGAAAACCATCTCCAA
AGCCAAAGGGCAGCCCCGAGAACCACAGGTGTACACCCTGCCCCCATCCCGGGAGGAGATGACC
AAGAACCAGGTCAGCCTGACCTGCCTGGTCAAAGGCTTCTATCCCAGCGACATCGCCGTGGAGTG
GGAGAGCAATGGGCAGCCGGAGAACAACTACAAGACCACGCCTCCCGTGCTGGACTCCGACGGC
TCCTTCTTCCTCTACAGCAAGCTCACCGTGGACAAGAGCAGGTGGCAGCAGGGGAACGTCITTCTC
ATGCTCCGTGATGCATGAGGCTCTGCACAACCACTACACGCAGAAGAGCCTCTCCCTGTCTCCGGG
TAAA
LIGHT CHAIN
SEQ ID NO: 287

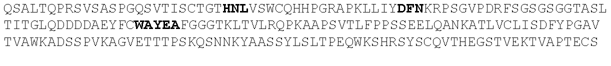

SEQ ID NO: 288
CAGTCTGCCCTGACTCAGCCTCGCTCAGTGTCCGCATCTCCTGGGCAGTCCGTCACCATTTCCTGC
ACTGGAACCCACAATTTGGTCTCTTGGTGTCAACATCACCCAGGCAGAGCCCCCAAATTATTAATT
TATGACTTCAATAAGAGGCCCTCAGGGGTCCCTGATCGCTTCTCTGGCTCCGGGTCTGGCGGCACG
GCCTCCCTGACCATCACTGGACTCCAGGATGACGATGACGCCGAATATTTTTGTTGGGCGTATGAA
GCTTTTGGCGGAGGGACCAAGTTGACCGTACTTCGTCAGCCCAAGGCTGCCCCCTCGGTCACTCTG
TTCCCGCCCTCCTCTGAGGAGCTTCAAGCCAACAAGGCCACACTGGTGTGTCTCATAAGTGACTTC
TACCCGGGAGCCGTGACAGTGGCTTGGAAAGCAGATAGCAGCCCCGTCAAGGCGGGAGTGGAGA
CCACCACACCCTCCAAACAAAGCAACAACAAGTACGCGGCCAGCAGCTATCTGAGCCTGACGCCT
GAGCAGTGGAAGTCCCACAGAAGCTACAGCTGCCAGGTCACGCATGAAGGGAGCACCGTGGAGA
AGACAGTGGCCCCTACAGAATGTTCA
>N49P75.1
HEAVY CHAIN
SEQ ID NO: 289

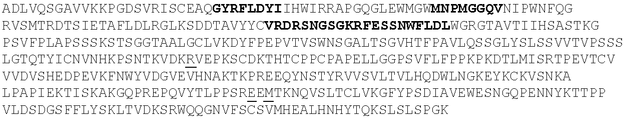

SEQ ID NO: 290
GCGGACTTGGTGCAGTCTGGGGCTGTGGTGAAGAAGCCTGGGGACTCAGTGAGGATCTCCTGTGA
GGCTCAAGGATACAGATTTCTTGACTACATCATTCACTGGATTCGACGAGCCCCTGGACAAGGCCT
TGAATGGATGGGATGGATGAATCCAATGGGCGGACAAGTAAACATTCCATGGAACTTTCAGGGTA
GGGTCTCCATGACCCGGGACACGTCCATCGAAACAGCATTTCTGGACTTAAGAGGACTAAAGTCT
GACGACACGGCCGTCTATTATTGCGTGAGAGATCGCAGTAATGGATCGGGAAAGCGATTCGAGTC
CTCCAATTGGTTCCTCGATCTGTGGGGCCGCGGGACTGCGGTCACTATTCATTCAGCCTCCACCAA
GGGCCCATCGGTCTTCCCCCTGGCACCCTCCTCCAAGAGCACCTCTGGGGGCACAGCGGCCCTGG
GCTGCCTGGTCAAGGACTACTTCCCCGAACCGGTGACGGTGTCGTGGAACTCAGGCGCCCTGACC
AGCGGCGTGCACACCTTCCCGGCTGTCCTACAGTCCTCAGGACTCTACTCCCTCAGCAGCGTGGTG
ACCGTGCCCTCCAGCAGCTTGGGCACCCAGACCTACATCTGCAACGTGAATCACAAGCCCAGCAA
CACCAAGGTGGACAAGAGAGTTGAGCCCAAATCTTGTGACAAAACTCACACATGCCCACCGTGCC
CAGCACCTGAACTCCTGGGGGGACCGTCAGTCTTCCTCTTCCCCCCAAAACCCAAGGACACCCTCA
TGATCTCCCGGACCCCTGAGGTCACATGCGTGGTGGTGGACGTGAGCCACGAAGACCCTGAGGTC
AAGTTCAACTGGTACGTGGACGGCGTGGAGGTGCATAATGCCAAGACAAAGCCGCGGGAGGAGC
AGTACAACAGCACGTACCGGGTGGTCAGCGTCCTCACCGTCCTGCACCAGGACTGGCTGAATGGC
AAGGAGTACAAGTGCAAGGTCTCCAACAAAGCCCTCCCAGCCCCCATCGAGAAAACCATCTCCAA
AGCCAAAGGGCAGCCCCGAGAACCACAGGTGTACACCCTGCCCCCATCCCGGGAGGAGATGACC
AAGAACCAGGTCAGCCTGACCTGCCTGGTCAAAGGCTTCTATCCCAGCGACATCGCCGTGGAGTG
GGAGAGCAATGGGCAGCCGGAGAACAACTACAAGACCACGCCTCCCGTGCTGGACTCCGACGGC
TCCTTCTTCCTCTACAGCAAGCTCACCGTGGACAAGAGCAGGTGGCAGCAGGGGAACGTCITTCTC
ATGCTCCGTGATGCATGAGGCTCTGCACAACCACTACACGCAGAAGAGCCTCTCCCTGTCTCCGGG
TAAA
LIGHT CHAIN
SEQ ID NO: 291

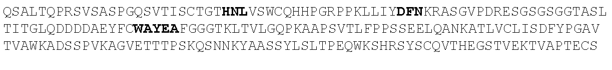

SEQ ID NO: 292
CAGTCTGCCCTGACTCAGCCTCGCTCAGTGTCCGCATCTCCTGGGCAGTCCGTCACCATTTCCTGC
ACTGGAACCCACAATTTGGTCTCTTGGTGTCAACATCACCCAGGCAGAGCCCCCAAATTATTAATT
TATGACTTCAATAAGAGGCCCTCAGGGGTCCCTGATCGCTTCTCTGGCTCCGGGTCTGGCGGCACG
GCCTCCCTGACCATCACTGGACTCCAGGATGACGATGACGCCGAATATTTTTGTTGGGCGTATGAA
GCTTTTGGCGGAGGGACCAAGTTGACCGTACTTGGTCAGCCCAAGGCTGCCCCCTCGGTCACTCTG
TTCCCGCCCTCCTCTGAGGAGCTTCAAGCCAACAAGGCCACACTGGTGTGTCTCATAAGTGACTTC
TACCCGGGAGCCGTGACAGTGGCTTGGAAAGCAGATAGCAGCCCCGTCAAGGCGGGAGTGGAGA
CCACCACACCCTCCAAACAAAGCAACAACAAGTACGCGGCCAGCAGCTATCTGAGCCTGACGCCT
GAGCAGTGGAAGTCCCACAGAAGCTACAGCTGCCAGGTCACGCATGAAGGGAGCACCGTGGAGA
AGACAGTGGCCCCTACAGAATGTTCA
>N49P9
HEAVY CHAIN
SEQ ID NO: 293

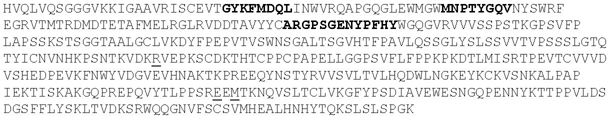

SEQ ID NO: 294
CACGTCCAATTGGTGCAGTCTGGAGGTGGGGTGAAGAAGATTGGGGCCGCTGTGAGGATCTCCTG
CGAGGTGACTGGATATAAATTCATGGACCAACTCATAAACTGGGTGCGGCAGGCCCCCGGTCAGG
GCCTTGAGTGGATGGGATGGATGAATCCAACATATGGACAAGTAAATTATTCATGGAGATTTGAA
GGAAGGGTCACCATGACCAGGGACATGGACACCGAGACGGCCTTCATGGAGTTGAGAGGACTGA
GAGTGGACGACACGGCCGTCTATTATTGCGCGAGGGGACCCTCTGGGGAAAATTATCCTTTTCACT
ATTGGGGCCAGGGTGTCCGAGTGGTCGTCTCGTCACCCTCCACCAAGGGCCCATCGGTCTTCCCCC
TGGCACCCTCCTCCAAGAGCACCTCTGGGGGCACAGCGGCCCTGGGCTGCCTGGTCAAGGACTAC
TTCCCCGAACCGGTGACGGTGTCGTGGAACTCAGGCGCCCTGACCAGCGGCGTGCACACCTTCCC
GGCTGTCCTACAGTCCTCAGGACTCTACTCCCTCAGCAGCGTGGTGACCGTGCCCTCCAGCAGCTT
GGGCACCCAGACCTACATCTGCAACGTGAATCACAAGCCCAGCAACACCAAGGTGGACAAGAGA
GTTGAGCCCAAATCTTGTGACAAAACTCACACATGCCCACCGTGCCCAGCACCTGAACTCCTGGG
GGGACCGTCAGTCTTCCTCTTCCCCCCAAAACCCAAGGACACCCTCATGATCTCCCGGACCCCTGA
GGTCACATGCGTGGTGGTGGACGTGAGCCACGAAGACCCTGAGGTCAAGTTCAACTGGTACGTGG
ACGGCGTGGAGGTGCATAATGCCAAGACAAAGCCGCGGGAGGAGCAGTACAACAGCACGTACCG
GGTGGTCAGCGTCCTCACCGTCCTGCACCAGGACTGGCTGAATGGCAAGGAGTACAAGTGCAAGG
TCTCCAACAAAGCCCTCCCAGCCCCCATCGAGAAAACCATCTCCAAAGCCAAAGGGCAGCCCCGA
GAACCACAGGTGTACACCCTGCCCCCATCCCGGGAGGAGATGACCAAGAACCAGGTCAGCCTGAC
CTGCCTGGTCAAAGGCTTCTATCCCAGCGACATCGCCGTGGAGTGGGAGAGCAATGGGCAGCCGG
AGAACAACTACAAGACCACGCCTCCCGTGCTGGACTCCGACGGCTCCTTCTTCCTCTACAGCAAGC
TCACCGTGGACAAGAGCAGGTGGCAGCAGGGGAACGTCTTCTCATGCTCCGTGATGCATGAGGCT
CTGCACAACCACTACACGCAGAAGAGCCTCTCCCTGTCTCCGGGTAAA
LIGHT CHAIN
SEQ ID NO: 295

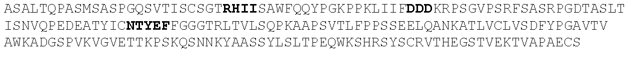

SEQ ID NO: 296
GCATCTGCCCTGACTCAGCCTGCCTCCATGTCTGCGTCCCCTGGACAGTCGGTAACCATCTCGTGC
TCTGGAACCAGACACATAATCTCTGCTTGGTTCCAACAATATCCAGGCAAACCACCCAAACTCATA
ATTTTTGACGACGATAAGCGTCCCTCTGGAGTTCCTAGTCGCTTCTCTGCCTCCAGGCCTGGCGAC
ACGGCCTCCCTGACAATCTCTAATGTTCAACCTGAGGACGAGGCGACGTACATTTGCAATACATAT
GAATTCTTTGGCGGAGGGACCAGATTGACCGTCCTAAGTCAGCCCAAGGCTGCCCCCTCGGTCACT
CTGTTCCCACCCTCCTCTGAGGAGCTTCAAGCCAACAAGGCCACACTGGTGTGTCTCGTAAGTGAC
TTCTACCCGGGAGCCGTGACAGTGGCCTGGAAGGCAGATGGCAGCCCCGTCAAGGTGGGAGTGGA
GACCACCAAACCCTCCAAACAAAGCAACAACAAGTATGCGGCCAGCAGCTACCTGAGCCTGACGC
CCGAGCAGTGGAAGTCCCACAGAAGCTACAGCTGCCGGGTCACGCATGAAGGGAGCACCGTGGA
GAAGACAGTGGCCCCTGCAGAATGCTCT
>N49P9.1
HEAVY CHAIN
SEQ ID NO: 297

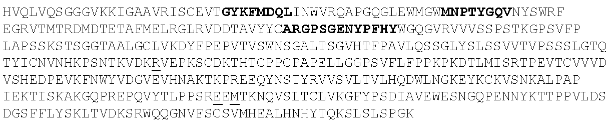

SEQ ID NO: 298
CACGTCCAATTGGTGCAGTCTGGAGGTGGGGTGAAGAAGATTGGGGCCGCTGTGAGGATCTCCTG
CGAGGTGACTGGATATAAATTCATGGACCAACTCATAAACTGGGTGCGGCAGGCCCCCGGTCAGG
GCCTTGAGTGGATGGGATGGATGAATCCAACATATGGACAAGTAAATTATTCATGGAGATTTGAA
GGAAGGGTCACCATGACCAGGGACATGGACACCGAGACGGCCTTCATGGAGTTGAGAGGACTGA
GAGTGGACGACACGGCCGTCTATTATTGCGCGAGGGGACCCTCTGGGGAAAATTATCCTTTTCACT
ATTGGGGCCAGGGTGTCCGAGTGGTCGTCTCGTCCCCTCCACCAAGGGCCCATCGGTCTTCCCCC
TGGCACCCTCCTCCAAGAGCACCTCTGGGGGCACAGCGGCCCTGGGCTGCCTGGTCAAGGACTAC
TTCCCCGAACCGGTGACGGTGTCGTGGAACTCAGGCGCCCTGACCAGCGGCGTGCACACCTTCCC
GGCTGTCCTACAGTCCTCAGGACTCTACTCCCTCAGCAGCGTGGTGACCGTGCCCTCCAGCAGCTT
GGGCACCCAGACCTACATCTGCAACGTGAATCACAAGCCCAGCAACACCAAGGTGGACAAGAGA
GTTGAGCCCAAATCTTGTGACAAAACTCACACATGCCCACCGTGCCCAGCACCTGAACTCCTGGG
GGGACCGTCAGTCTTCCTCTTCCCCCCAAAACCCAAGGACACCCTCATGATCTCCCGGACCCCTGA
GGTCACATGCGTGGTGGTGGACGTGAGCCACGAAGACCCTGAGGTCAAGTTCAACTGGTACGTGG
ACGGCGTGGAGGTGCATAATGCCAAGACAAAGCCGCGGGAGGAGCAGTACAACAGCACGTACCG
GGTGGTCAGCGTCCTCACCGTCCTGCACCAGGACTGGCTGAATGGCAAGGAGTACAAGTGCAAGG
TCTCCAACAAAGCCCTCCCAGCCCCCATCGAGAAAACCATCTCCAAAGCCAAAGGGCAGCCCCGA
GAACCACAGGTGTACACCCTGCCCCCATCCCGGGAGGAGATGACCAAGAACCAGGTCAGCCTGAC
CTGCCTGGTCAAAGGCTTCTATCCCAGCGACATCGCCGTGGAGTGGGAGAGCAATGGGCAGCCGG
AGAACAACTACAAGACCACGCCTCCCGTGCTGGACTCCGACGGCTCCTTCTTCCTCTACAGCAAGC
TCACCGTGGACAAGAGCAGGTGGCAGCAGGGGAACGTCTTCTCATGCTCCGTGATGCATGAGGCT
CTGCACAACCACTACACGCAGAAGAGCCTCTCCCTGTCTCCGGGTAAA
LIGHT CHAIN
SEQ ID NO: 299

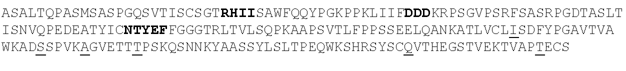

SEQ ID NO: 300
GCATCTGCCCTGACTCAGCCTGCCTCCATGTCTGCGTCCCCTGGACAGTCGGTAACCATCTCGTGC
TCTGGAACCAGACACATAATCTCTGCTTGGTTCCAACAATATCCAGGCAAACCACCCAAACTCATA
ATTTTTGACGACGATAAGCGTCCCTCTGGAGTTCCTAGTCGCTTCTCTGCCTCCAGGCCTGGCGAC
ACGGCCTCCCTGACAATCTCTAATGTTCAACCTGAGGACGAGGCGACGTACATTTGCAATACATAT
GAATTCTTTGGCGGAGGGACCAGATTGACCGTCCTAAGTCAGCCCAAGGCTGCCCCCTCGGTCACT
CTGTTCCCGCCCTCCTCTGAGGAGCTTCAAGCCAACAAGGCCACACTGGTGTGTCTCATAAGTGAC
TTCTACCCGGGAGCCGTGACAGTGGCTTGGAAAGCAGATAGCAGCCCCGTCAAGGCGGGAGTGGA
GACCACCACACCCTCCAAACAAAGCAACAACAAGTACGCGGCCAGCAGCTATCTGAGCCTGACGC
CTGAGCAGTGGAAGTCCCACAGAAGCTACAGCTGCCAGGTCACGCATGAAGGGAGCACCGTGGA
GAAGACAGTGGCCCCTACAGAATGTTCA
>N49P9.2
HEAVY CHAIN
SEQ ID NO: 301

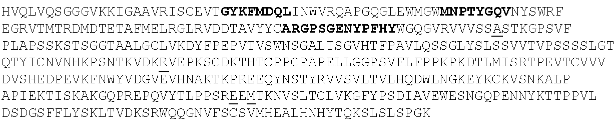

SEQ ID NO: 302
CACGTCCAATTGGTGCAGTCTGGAGGTGGGGTGAAGAAGATTGGGGCCGCTGTGAGGATCTCCTG
CGAGGTGACTGGATATAAATTCATGGACCAACTCATAAACTGGGTGCGGCAGGCCCCCGGTCAGG
GCCTTGAGTGGATGGGATGGATGAATCCAACATATGGACAAGTAAATTATTCATGGAGATTTGAA
GGAAGGGTCACCATGACCAGGGACATGGACACCGAGACGGCCTTCATGGAGTTGAGAGGACTGA
GAGTGGACGACACGGCCGTCTATTATTGCGCGAGGGGACCCTCTGGGGAAAATTATCCTTTTCACT
ATTGGGGCCAGGGTGTCCGAGTGGTCGTCTCGTCATCCTCCAACCAAGGGCCCATCGGTCTTCCCCC
TGGCACCCTCCTCCAAGAGCACCTCTGGGGGCACAGCGGCCCTGGGCTGCCTGGTCAAGGACTAC
TTCCCCGAACCGGTGACGGTGTCGTGGAACTCAGGCGCCCTGACCAGCGGCGTGCACACCTTCCC
GGCTGTCCTACAGTCCTCAGGACTCTACTCCCTCAGCAGCGTGGTGACCGTGCCCTCCAGCAGCTT
GGGCACCCAGACCTACATCTGCAACGTGAATCACAAGCCCAGCAACACCAAGGTGGACAAGAGA
GTTGAGCCCAAATCTTGTGACAAAACTCACACATGCCCACCGTGCCCAGCACCTGAACTCCTGGG
GGGACCGTCAGTCTTCCTCTTCCCCCCAAAACCCAAGGACACCCTCATGATCTCCCGGACCCCTGA
GGTCACATGCGTGGTGGTGGACGTGAGCCACGAAGACCCTGAGGTCAAGTTCAACTGGTACGTGG
ACGGCGTGGAGGTGCATAATGCCAAGACAAAGCCGCGGGAGGAGCAGTACAACAGCACGTACCG
GGTGGTCAGCGTCCTCACCGTCCTGCACCAGGACTGGCTGAATGGCAAGGAGTACAAGTGCAAGG
TCTCCAACAAAGCCCTCCCAGCCCCCATCGAGAAAACCATCTCCAAAGCCAAAGGGCAGCCCCGA
GAACCACAGGTGTACACCCTGCCCCCATCCCGGGAGGAGATGACCAAGAACCAGGTCAGCCTGAC
CTGCCTGGTCAAAGGCTTCTATCCCAGCGACATCGCCGTGGAGTGGGAGAGCAATGGGCAGCCGG
AGAACAACTACAAGACCACGCCTCCCGTGCTGGACTCCGACGGCTCCTTCTTCCTCTACAGCAAGC
TCACCGTGGACAAGAGCAGGTGGCAGCAGGGGAACGTCTTCTCATGCTCCGTGATGCATGAGGCT
CTGCACAACCACTACACGCAGAAGAGCCTCTCCCTGTCTCCGGGTAAA
LIGHT CHAIN
SEQ ID NO: 303

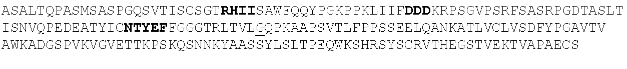

SEQ ID NO: 304
GCATCTGCCCTGACTCAGCCTGCCTCCATGTCTGCGTCCCCTGGACAGTCGGTAACCATCTCGTGC
TCTGGAACCAGACACATAATCTCTGCTTGGTTCCAACAATATCCAGGCAAACCACCCAAACTCATA
ATTTTTGACGACGATAAGCGTCCCTCTGGAGTTCCTAGTCGCTTCTCTGCCTCCAGGCCTGGCGAC
ACGGCCTCCCTGACAATCTCTAATGTTCAACCTGAGGACGAGGCGACGTACATTTGCAATACATAT
GAATTCTTTGGCGGAGGGACCAGATTGACCGTCCTAGGTCAGCCCAAGGCTGCCCCCTCGGTCACT
CTGTTCCCACCCTCCTCTGAGGAGCTTCAAGCCAACAAGGCCACACTGGTGTGTCTCGTAAGTGAC
TTCTACCCGGGAGCCGTGACAGTGGCCTGGAAGGCAGATGGCAGCCCCGTCAAGGTGGGAGTGGA
GACCACCAAACCCTCCAAACAAAGCAACAACAAGTATGCGGCCAGCAGCTACCTGAGCCTGACGC
CCGAGCAGTGGAAGTCCCACAGAAGCTACAGCTGCCGGGTCACGCATGAAGGGAGCACCGTGGA
GAAGACAGTGGCCCCTGCAGAATGCTCT
>N49P9i7
HEAVY CHAIN
SEQ ID NO: 305

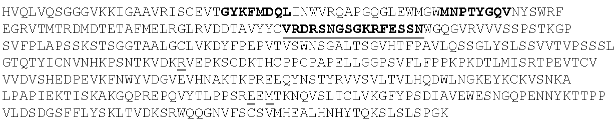

SEQ ID NO: 306
CACGTCCAATTGGTGCAGTCTGGAGGTGGGGTGAAGAAGATTGGGGCCGCTGTGAGGATCTCCTG
CGAGGTGACTGGATATAAATTCATGGACCAACTCATAAACTGGGTGCGGCAGGCCCCCGGTCAGG
GCCTTGAGTGGATGGGATGGATGAATCCAACATATGGACAAGTAAATTATTCATGGAGATTTGAA
GGAAGGGTCACCATGACCAGGGACATGGACACCGAGACGGCCTTCATGGAGTTGAGAGGACTGA
GAGTGGACGACACGGCCGTCTATTATTGCGTGAGAGATCGCAGTAATGGATCGGGAAAGCGATTC
GAGTCCTCCAATTGGGGCCAGGGTGTCCGAGTGGTCGTCTCGTCACCCTCCACCAAGGGCCCATCG
GTCTTCCCCCTGGCACCCTCCTCCAAGAGCACCTCTGGGGGCACAGCGGCCCTGGGCTGCCTGGTC
AAGGACTACTTCCCCGAACCGGTGACGGTGTCGTGGAACTCAGGCGCCCTGACCAGCGGCGTGCA
CACCTTCCCGGCTGTCCTACAGTCCTCAGGACTCTACTCCCTCAGCAGCGTGGTGACCGTGCCCTC
CAGCAGCTTGGGCACCCAGACCTACATCTGCAACGTGAATCACAAGCCCAGCAACACCAAGGTGG
ACAAGAGAGTTGAGCCCAAATCTTGTGACAAAACTCACACATGCCCACCGTGCCCAGCACCTGAA
CTCCTGGGGGGACCGTCAGTCTTCCTCTTCCCCCCAAAACCCAAGGACACCCTCATGATCTCCCGG
ACCCCTGAGGTCACATGCGTGGTGGTGGACGTGAGCCACGAAGACCCTGAGGTCAAGTTCAACTG
GTACGTGGACGGCGTGGAGGTGCATAATGCCAAGACAAAGCCGCGGGAGGAGCAGTACAACAGC
ACGTACCGGGTGGTCAGCGTCCTCACCGTCCTGCACCAGGACTGGCTGAATGGCAAGGAGTACAA
GTGCAAGGTCTCCAACAAAGCCCTCCCAGCCCCCATCGAGAAAACCATCTCCAAAGCCAAAGGGC
AGCCCCGAGAACCACAGGTGTACACCCTGCCCCCATCCCGGGAGGAGATGACCAAGAACCAGGTC
AGCCTGACCTGCCTGGTCAAAGGCTTCTATCCCAGCGACATCGCCGTGGAGTGGGAGAGCAATGG
GCAGCCGGAGAACAACTACAAGACCACGCCTCCCGTGCTGGACTCCGACGGCTCCTTCTTCCTCTA
CAGCAAGCTCACCGTGGACAAGAGCAGGTGGCAGCAGGGGAACGTCTTCTCATGCTCCGTGATGC
ATGAGGCTCTGCACAACCACTACACGCAGAAGAGCCTCTCCCTGTCTCCGGGTAAA
LIGHT CHAIN
SEQ ID NO: 307

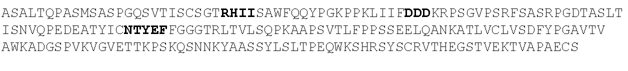

SEQ ID NO: 308
GCATCTGCCCTGACTCAGCCTGCCTCCATGTCTGCGTCCCCTGGACAGTCGGTAACCATCTCGTGC
TCTGGAACCAGACACATAATCTCTGCTTGGTTCCAACAATATCCAGGCAAACCACCCAAACTCATA
ATTTTTGACGACGATAAGCGTCCCTCTGGAGTTCCTAGTCGCTTCTCTGCCTCCAGGCCTGGCGAC
ACGGCCTCCCTGACAATCTCTAATGTTCAACCTGAGGACGAGGCGACGTACATTTGCAATACATAT
GAATTCTTTGGCGGAGGGACCAGATTGACCGTCCTAAGTCAGCCCAAGGCTGCCCCCTCGGTCACT
CTGTTCCCACCCTCCTCTGAGGAGCTTCAAGCCAACAAGGCCACACTGGTGTGTCTCGTAAGTGAC
TTCTACCCGGGAGCCGTGACAGTGGCCTGGAAGGCAGATGGCAGCCCCGTCAAGGTGGGAGTGGA
GACCACCAAACCCTCCAAACAAAGCAACAACAAGTATGCGGCCAGCAGCTACCTGAGCCTGACGC
CCGAGCAGTGGAAGTCCCACAGAAGCTACAGCTGCCGGGTCACGCATGAAGGGAGCACCGTGGA
GAAGACAGTGGCCCCTGCAGAATGCTCT
>N49P9i7H1
HEAVY CHAIN
SEQ ID NO: 309

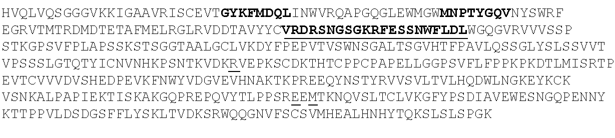

SEQ ID NO: 310
CACGTCCAATTGGTGCAGTCTGGAGGTGGGGTGAAGAAGATTGGGGCCGCTGTGAGGATCTCCTG
CGAGGTGACTGGATATAAATTCATGGACCAACTCATAAACTGGGTGCGGCAGGCCCCCGGTCAGG
GCCTTGAGTGGATGGGATGGATGAATCCAACATATGGACAAGTAAATTATTCATGGAGATTTGAA
GGAAGGGTCACCATGACCAGGGACATGGACACCGAGACGGCCTTCATGGAGTTGAGAGGACTGA
GAGTGGACGACACGGCCGTCTATTATTGCGTGAGAGATCGCAGTAATGGATCGGGAAAGCGATTC
GAGTCCTCCAATTGGTTCCTCGATCTGTGGGGCCAGGGTGTCCGAGTGGTCGTCTCGTCACCCTCC
ACCAAGGGCCCATCGGTCTTCCCCCTGGCACCCTCCTCCAAGAGCACCTCTGGGGGCACAGCGGC
CCTGGGCTGCCTGGTCAAGGACTACTTCCCCGAACCGGTGACGGTGTCGTGGAACTCAGGCGCCC
TGACCAGCGGCGTGCACACCTTCCCGGCTGTCCTACAGTCCTCAGGACTCTACTCCCTCAGCAGCG
TGGTGACCGTGCCCTCCAGCAGCTTGGGCACCCAGACCTACATCTGCAACGTGAATCACAAGCCC
AGCAACACCAAGGTGGACAAGAGAGTTGAGCCCAAATCTTGTGACAAAACTCACACATGCCCACC
GTGCCCAGCACCTGAACTCCTGGGGGGACCGTCAGTCTTCCTCTTCCCCCCAAAACCCAAGGACAC
CCTCATGATCTCCCGGACCCCTGAGGTCACATGCGTGGTGGTGGACGTGAGCCACGAAGACCCTG
AGGTCAAGTTCAACTGGTACGTGGACGGCGTGGAGGTGCATAATGCCAAGACAAAGCCGCGGGA
GGAGCAGTACAACAGCACGTACCGGGTGGTCAGCGTCCTCACCGTCCTGCACCAGGACTGGCTGA
ATGGCAAGGAGTACAAGTGCAAGGTCTCCAACAAAGCCCTCCCAGCCCCCATCGAGAAAACCATC
TCCAAAGCCAAAGGGCAGCCCCGAGAACCACAGGTGTACACCCTGCCCCCATCCCGGGAGGAGAT
GACCAAGAACCAGGTCAGCCTGACCTGCCTGGTCAAAGGCTTCTATCCCAGCGACATCGCCGTGG
AGTGGGAGAGCAATGGGCAGCCGGAGAACAACTACAAGACCACGCCTCCCGTGCTGGACTCCGA
CGGCTCCTTCTTCCTCTACAGCAAGCTCACCGTGGACAAGAGCAGGTGGCAGCAGGGGAACGTCT
TCTCATGCTCCGTGATGCATGAGGCTCTGCACAACCACTACACGCAGAAGAGCCTCTCCCTGTCTC
CGGGTAAA
LIGHT CHAIN
SEQ ID NO: 311

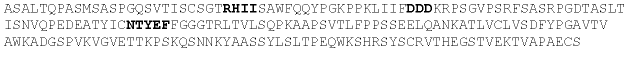

SEQ ID NO: 312
GCATCTGCCCTGACTCAGCCTGCCTCCATGTCTGCGTCCCCTGGACAGTCGGTAACCATCTCGTGC
TCTGGAACCAGACACATAATCTCTGCTTGGTTCCAACAATATCCAGGCAAACCACCCAAACTCATA
ATTTTTGACGACGATAAGCGTCCCTCTGGAGTTCCTAGTCGCTTCTCTGCCTCCAGGCCTGGCGAC
ACGGCCTCCCTGACAATCTCTAATGTTCAACCTGAGGACGAGGCGACGTACATTTGCAATACATAT
GAATTCTTTGGCGGAGGGACCAGATTGACCGTCCTAAGTCAGCCCAAGGCTGCCCCCTCGGTCACT
CTGTTCCCACCCTCCTCTGAGGAGCTTCAAGCCAACAAGGCCACACTGGTGTGTCTCGTAAGTGAC
TTCTACCCGGGAGCCGTGACAGTGGCCTGGAAGGCAGATGGCAGCCCCGTCAAGGTGGGAGTGGA
GACCACCAAACCCTCCAAACAAAGCAACAACAAGTATGCGGCCAGCAGCTACCTGAGCCTGACGC
CCGAGCAGTGGAAGTCCCACAGAAGCTACAGCTGCCGGGTCACGCATGAAGGGAGCACCGTGGA
GAAGACAGTGGCCCCTGCAGAATGCTCT
>N49P9i7H2
HEAVY CHAIN
SEQ ID NO: 313

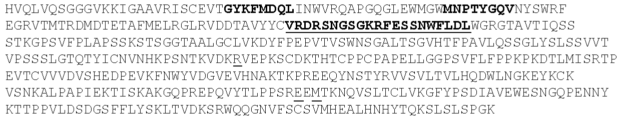

SEQ ID NO: 314
CACGTCCAATTGGTGCAGTCTGGAGGTGGGGTGAAGAAGATTGGGGCCGCTGTGAGGATCTCCTG
CGAGGTGACTGGATATAAATTCATGGACCAACTCATAAACTGGGTGCGGCAGGCCCCCGGTCAGG
GCCTTGAGTGGATGGGATGGATGAATCCAACATATGGACAAGTAAATTATTCATGGAGATTTGAA
GGAAGGGTCACCATGACCAGGGACATGGACACCGAGACGGCCTTCATGGAGTTGAGAGGACTGA
GAGTGGACGACACGGCCGTCTATTATTGCGTGAGAGATCGCAGTAATGGATCGGGAAAGCGATTC
GAGTCCTCCAATTGGTTCCTCGATCTGTGGGGCCGTGGGACTGCGGTCACAATTCAATCATCCTCC
ACCAAGGGCCCATCGGTCTTCCCCCTGGCACCCTCCTCCAAGAGCACCTCTGGGGGCACAGCGGC
CCTGGGCTGCCTGGTCAAGGACTACTTCCCCGAACCGGTGACGGTGTCGTGGAACTCAGGCGCCC
TGACCAGCGGCGTGCACACCTTCCCGGCTGTCCTACAGTCCTCAGGACTCTACTCCCTCAGCAGCG
TGGTGACCGTGCCCTCCAGCAGCTTGGGCACCCAGACCTACATCTGCAACGTGAATCACAAGCCC
AGCAACACCAAGGTGGACAAGAGAGTTGAGCCCAAATCTTGTGACAAAACTCACACATGCCCACC
GTGCCCAGCACCTGAACTCCTGGGGGGACCGTCAGTCTTCCTCTTCCCCCCAAAACCCAAGGACAC
CCTCATGATCTCCCGGACCCCTGAGGTCACATGCGTGGTGGTGGACGTGAGCCACGAAGACCCTG
AGGTCAAGTTCAACTGGTACGTGGACGGCGTGGAGGTGCATAATGCCAAGACAAAGCCGCGGGA
GGAGCAGTACAACAGCACGTACCGGGTGGTCAGCGTCCTCACCGTCCTGCACCAGGACTGGCTGA
ATGGCAAGGAGTACAAGTGCAAGGTCTCCAACAAAGCCCTCCCAGCCCCCATCGAGAAAACCATC
TCCAAAGCCAAAGGGCAGCCCCGAGAACCACAGGTGTACACCCTGCCCCCATCCCGGGAGGAGAT
GACCAAGAACCAGGTCAGCCTGACCTGCCTGGTCAAAGGCTTCTATCCCAGCGACATCGCCGTGG
AGTGGGAGAGCAATGGGCAGCCGGAGAACAACTACAAGACCACGCCTCCCGTGCTGGACTCCGA
CGGCTCCTTCTTCCTCTACAGCAAGCTCACCGTGGACAAGAGCAGGTGGCAGCAGGGGAACGTCT
TCTCATGCTCCGTGATGCATGAGGCTCTGCACAACCACTACACGCAGAAGAGCCTCTCCCTGTCTC
CGGGTAAA
LIGHT CHAIN
SEQ ID NO: 315

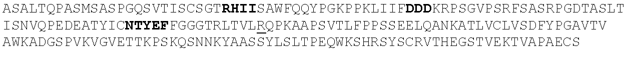

SEQ ID NO: 316
GCATCTGCCCTGACTCAGCCTGCCTCCATGTCTGCGTCCCCTGGACAGTCGGTAACCATCTCGTGC
TCTGGAACCAGACACATAATCTCTGCTTGGTTCCAACAATATCCAGGCAAACCACCCAAACTCATA
ATTTTTGACGACGATAAGCGTCCCTCTGGAGTTCCTAGTCGCTTCTCTGCCTCCAGGCCTGGCGAC
ACGGCCTCCCTGACAATCTCTAATGTTCAACCTGAGGACGAGGCGACGTACATTTGCAATACATAT
GAATTCTTTGGCGGAGGGACCAGATTGACCGTCCTACGTCAGCCCAAGGCTGCCCCCTCGGTCACT
CTGTTCCCACCCTCCTCTGAGGAGCTTCAAGCCAACAAGGCCACACTGGTGTGTCTCGTAAGTGAC
TTCTACCCGGGAGCCGTGACAGTGGCCTGGAAGGCAGATGGCAGCCCCGTCAAGGTGGGAGTGGA
GACCACCAAACCCTCCAAACAAAGCAACAACAAGTATGCGGCCAGCAGCTACCTGAGCCTGACGC
CCGAGCAGTGGAAGTCCCACAGAAGCTACAGCTGCCGGGTCACGCATGAAGGGAGCACCGTGGA
GAAGACAGTGGCCCCTGCAGAATGCTCT
>N49P22
HEAVY CHAIN
SEQ ID NO: 317

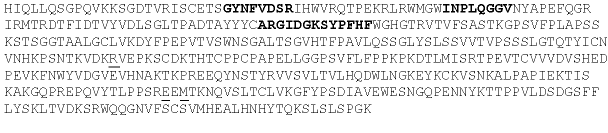

SEQ ID NO: 318
CACATTCAGTTGCTACAGTCGGGGCCTCAAGTTAAGAAGTCTGGGGACACAGTGAGAATCTCCTG
TGAGACCTCTGGATATAACTTCGTCGACTCCCGTATCCACTGGGTCCGACAGACCCCGGAAAAAC
GTCTCAGATGGATGGGCTGGATCAATCCTCTCCAAGGTGGTGTGAATTACGCGCCGGAATTTCAGG
GCAGAATCAGGATGACCAGGGACACATTTATAGACACAGTTTACGTGGACCTGAGCGGACTGACA
CCGGCCGACACGGCCTATTATTACTGCGCGCGAGGGATCGATGGCAAGTCTTACCCCTTTCATTTC
TGGGGCCACGGAACCCGGGTCACCGTCTTCTCGGCCTCCACCAAGGGCCCATCGGTCTTCCCCCTG
GCACCCTCCTCCAAGAGCACCTCTGGGGGCACAGCGGCCCTGGGCTGCCTGGTCAAGGACTACTT
CCCCGAACCGGTGACGGTGTCGTGGAACTCAGGCGCCCTGACCAGCGGCGTGCACACCTTCCCGG
CTGTCCTACAGTCCTCAGGACTCTACTCCCTCAGCAGCGTGGTGACCGTGCCCTCCAGCAGCTTGG
GCACCCAGACCTACATCTGCAACGTGAATCACAAGCCCAGCAACACCAAGGTGGACAAGAGAGTT
GAGCCCAAATCTTGTGACAAAACTCACACATGCCCACCGTGCCCAGCACCTGAACTCCTGGGGGG
ACCGTCAGTCTTCCTCTTCCCCCCAAAACCCAAGGACACCCTCATGATCTCCCGGACCCCTGAGGT
CACATGCGTGGTGGTGGACGTGAGCCACGAAGACCCTGAGGTCAAGTTCAACTGGTACGTGGACG
GCGTGGAGGTGCATAATGCCAAGACAAAGCCGCGGGAGGAGCAGTACAACAGCACGTACCGGGT
GGTCAGCGTCCTCACCGTCCTGCACCAGGACTGGCTGAATGGCAAGGAGTACAAGTGCAAGGTCT
CCAACAAAGCCCTCCCAGCCCCCATCGAGAAAACCATCTCCAAAGCCAAAGGGCAGCCCCGAGA
ACCACAGGTGTACACCCTGCCCCCATCCCGGGAGGAGATGACCAAGAACCAGGTCAGCCTGACCT
GCCTGGTCAAAGGCTTCTATCCCAGCGACATCGCCGTGGAGTGGGAGAGCAATGGGCAGCCGGAG
AACAACTACAAGACCACGCCTCCCGTGCTGGACTCCGACGGCTCCTTCTTCCTCTACAGCAAGCTC
ACCGTGGACAAGAGCAGGTGGCAGCAGGGGAACGTCTTCTCATGCTCCGTGATGCATGAGGCTCT
GCACAACCACTACACGCAGAAGAGCCTCTCCCTGTCTCCGGGTAAA
LIGHT CHAIN
SEQ ID NO: 319

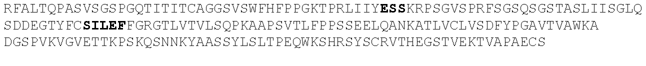

SEQ ID NO: 320
CGATTTGCCCTGACTCAACCTGCCTCCGTGTCTGGGTCTCCTGGACAGACGATCACCATAACCTGC
GCTGGAGGCAGCGTCTCCTGGTTTCATTTCCCTCCAGGCAAAACCCCCAGACTCATTATTTATGAG
TCTTCTAAGCGACCCTCTGGGGTCTCTCCTCGATTCTCTGGGTCCCAGTCTGGCAGCACGGCCTCCC
TTATAATTTCTGGCCTCCAGTCTGATGACGAAGGGACATACTTCTGTTCTATTCTTGAATTTTTCGG
CAGAGGGACTCTTGTCACCGTCCTGAGTCAGCCCAAGGCTGCCCCCTCGGTCACTCTGTTCCCACC
CTCCTCTGAGGAGCTTCAAGCCAACAAGGCCACACTGGTGTGTCTCGTAAGTGACTTCTACCCGGG
AGCCGTGACAGTGGCCTGGAAGGCAGATGGCAGCCCCGTCAAGGTGGGAGTGGAGACCACCAAA
CCCTCCAAACAAAGCAACAACAAGTATGCGGCCAGCAGCTACCTGAGCCTGACGCCCGAGCAGTG
GAAGTCCCACAGAAGCTACAGCTGCCGGGTCACGCATGAAGGGAGCACCGTGGAGAAGACAGTG
GCCCCTGCAGAATGCTCT
>N49P23
HEAVY CHAIN
SEQ ID NO: 321

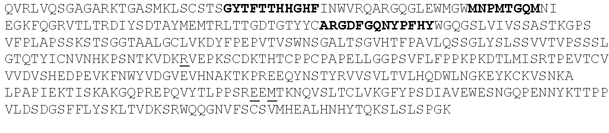

SEQ ID NO: 322
CAGGTGCGCTTGGTGCAGTCTGGGGCTGGGGCGAGGAAGACTGGGGCCTCAATGAAACTTTCCTG
CTCGACCTCTGGATACACCTTCACCACTCATCACGGCCACTTCATAAATTGGGTGCGACAGGCCCG
TGGACAAGGGCTTGAGTGGATGGGGTGGATGAATCCCATGACTGGGCAGATGAATATTGAGGGGA
AATTTCAGGGCAGAGTCACCCTCACTCGAGACATATACAGTGACACGGCTTACATGGAAATGACC
AGACTGACAACTGGCGACACGGGCACTTATTACTGTGCGCGAGGCGATTTCGGACAGAATTATCC
CTTTCATTATTGGGGCCAGGGAAGCCTGGTCATCGTCTCCTCGGCCTCCACCAAGGGCCCATCGGT
CTTCCCCCTGGCACCCTCCTCCAAGAGCACCTCTGGGGGCACAGCGGCCCTGGGCTGCCTGGTCAA
GGACTACTTCCCCGAACCGGTGACGGTGTCGTGGAACTCAGGCGCCCTGACCAGCGGCGTGCACA
CCTTCCCGGCTGTCCTACAGTCCTCAGGACTCTACTCCCTCAGCAGCGTGGTGACCGTGCCCTCCA
GCAGCTTGGGCACCCAGACCTACATCTGCAACGTGAATCACAAGCCCAGCAACACCAAGGTGGAC
AAGAGAGTTGAGCCCAAATCTTGTGACAAAACTCACACATGCCCACCGTGCCCAGCACCTGAACT
CCTGGGGGGACCGTCAGTCTTCCTCTTCCCCCCAAAACCCAAGGACACCCTCATGATCTCCCGGAC
CCCTGAGGTCACATGCGTGGTGGTGGACGTGAGCCACGAAGACCCTGAGGTCAAGTTCAACTGGT
ACGTGGACGGCGTGGAGGTGCATAATGCCAAGACAAAGCCGCGGGAGGAGCAGTACAACAGCAC
GTACCGGGTGGTCAGCGTCCTCACCGTCCTGCACCAGGACTGGCTGAATGGCAAGGAGTACAAGT
GCAAGGTCTCCAACAAAGCCCTCCCAGCCCCCATCGAGAAAACCATCTCCAAAGCCAAAGGGCAG
CCCCGAGAACCACAGGTGTACACCCTGCCCCCATCCCGGGAGGAGATGACCAAGAACCAGGTCAG
CCTGACCTGCCTGGTCAAAGGCTTCTATCCCAGCGACATCGCCGTGGAGTGGGAGAGCAATGGGC
AGCCGGAGAACAACTACAAGACCACGCCTCCCGTGCTGGACTCCGACGGCTCCTTCTTCCTCTACA
GCAAGCTCACCGTGGACAAGAGCAGGTGGCAGCAGGGGAACGTCTTCTCATGCTCCGTGATGCAT
GAGGCTCTGCACAACCACTACACGCAGAAGAGCCTCTCCCTGTCTCCGGGTAAA
LIGHT CHAIN
SEQ ID NO: 323

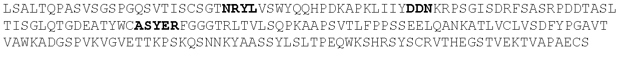

SEQ ID NO: 324
CTGTCTGCCCTGACTCAGCCTGCCTCCGTGTCTGGGTCTCCTGGGCAGTCGGTCACCATCTCCTGCT
CTGGAACGAACCGTTACCTTGTCTCCTGGTATCAACAACACCCTGACAAAGCCCCCAAACTCATCA
TTTATGACGACAATAAGCGGCCCTCAGGAATTTCTGATCGCTTCTCAGCCTCCAGGCCTGACGACA
CGGCCTCCCTGACAATCTCTGGACTCCAGACTGGGGACGAGGCTACTTATTGGTGTGCCTCATATG
AACGTTTTGGCGGCGGGACGAGGCTGACCGTCCTTAGTCAGCCCAAGGCTGCCCCCTCGGTCACTC
TGTTCCCACCCTCCTCTGAGGAGCTTCAAGCCAACAAGGCCACACTGGTGTGTCTCGTAAGTGACT
TCTACCCGGGAGCCGTGACAGTGGCCTGGAAGGCAGATGGCAGCCCCGTCAAGGTGGGAGTGGA
GACCACCAAACCCTCCAAACAAAGCAACAACAAGTATGCGGCCAGCAGCTACCTGAGCCTGACGC
CCGAGCAGTGGAAGTCCCACAGAAGCTACAGCTGCCGGGTCACGCATGAAGGGAGCACCGTGGA
GAAGACAGTGGCCCCTGCAGAATGCTCT
>N49P9.3
HEAVY CHAIN
SEQ ID NO: 325

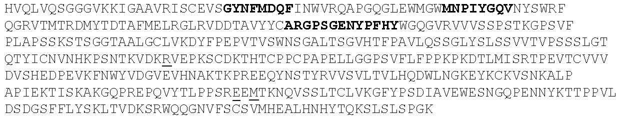

SEQ ID NO: 326
CACGTCCAATTGGTGCAGTCTGGAGGTGGGGTGAAGAAGATTGGGGCCGCTGTGAGGATCTCCTG
CGAGGTGTCTGGATACAACTTCATGGACCAATTCATAAATTGGGTGCGACAGGCCCCCGGTCAGG
GCCTTGAGTGGATGGGATGGATGAACCCAATATATGGACAAGTAAATTATTCATGGAGATTTCAA
GGAAGGGTCACCATGACCAGGGACATGTACACCGACACGGCCTTCATGGAGTTGAGAGGACTGAG
AGTGGACGACACGGCCGTCTATTATTGCGCGAGGGGACCCTCTGGGGAAAATTATCCTTTTCACTA
TTGGGGCCAGGGTGTCCGAGTGGTCGTCTCGTCACCCTCCACCAAGGGCCCATCGGTCTTCCCCCT
GGCACCCTCCTCCAAGAGCACCTCTGGGGGCACAGCGGCCCTGGGCTGCCTGGTCAAGGACTACT
TCCCCGAACCGGTGACGGTGTCGTGGAACTCAGGCGCCCTGACCAGCGGCGTGCACACCTTCCCG
GCTGTCCTACAGTCCTCAGGACTCTACTCCCTCAGCAGCGTGGTGACCGTGCCCTCCAGCAGCTTG
GGCACCCAGACCTACATCTGCAACGTGAATCACAAGCCCAGCAACACCAAGGTGGACAAGAGAG
TTGAGCCCAAATCTTGTGACAAAACTCACACATGCCCACCGTGCCCAGCACCTGAACTCCTGGGG
GGACCGTCAGTCTTCCTCTTCCCCCCAAAACCCAAGGACACCCTCATGATCTCCCGGACCCCTGAG
GTCACATGCGTGGTGGTGGACGTGAGCCACGAAGACCCTGAGGTCAAGTTCAACTGGTACGTGGA
CGGCGTGGAGGTGCATAATGCCAAGACAAAGCCGCGGGAGGAGCAGTACAACAGCACGTACCGG
GTGGTCAGCGTCCTCACCGTCCTGCACCAGGACTGGCTGAATGGCAAGGAGTACAAGTGCAAGGT
CTCCAACAAAGCCCTCCCAGCCCCCATCGAGAAAACCATCTCCAAAGCCAAAGGGCAGCCCCGAG
AACCACAGGTGTACACCCTGCCCCCATCCCGGGAGGAGATGACCAAGAACCAGGTCAGCCTGACC
TGCCTGGTCAAAGGCTTCTATCCCAGCGACATCGCCGTGGAGTGGGAGAGCAATGGGCAGCCGGA
GAACAACTACAAGACCACGCCTCCCGTGCTGGACTCCGACGGCTCCTTCTTCCTCTACAGCAAGCT
CACCGTGGACAAGAGCAGGTGGCAGCAGGGGAACGTCTTCTCATGCTCCGTGATGCATGAGGCTC
TGCACAACCACTACACGCAGAAGAGCCTCTCCCTGTCTCCGGGTAAA
LIGHT CHAIN
SEQ ID NO: 327

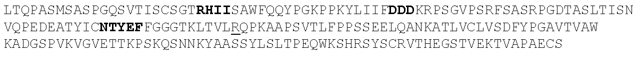

SEQ ID NO: 328
CTGACTCAGCCTGCCTCCATGTCTGCGTCCCCTGGACAGTCGGTAACCATCTCGTGCTCTGGAACC
AGACACATAATCTCTGCTTGGTTCCAACAATATCCAGGCAAACCACCCAAACTCATAATTTTTGAC
GACGATAAGCGTCCCTCTGGAGTTCCTAGTCGCTTCTCTGCCTCCAGGCCTGGCGACACGGCCTCC
CTGACAATCTCTAATGTTCAACCTGAGGACGAGGCGACATACATTTGCAATACATATGAATTCTTT
GGCGGAGGGACCAAATTGACCGTCCTACGTCAGCCCAAGGCTGCCCCCTCGGTCACTCTGTTCCCA
CCCTCCTCTGAGGAGCTTCAAGCCAACAAGGCCACACTGGTGTGTCTCGTAAGTGACTTCTACCCG
GGAGCCGTGACAGTGGCCTGGAAGGCAGATGGCAGCCCCGTCAAGGTGGGAGTGGAGACCACCA
AACCCTCCAAACAAAGCAACAACAAGTATGCGGCCAGCAGCTACCTGAGCCTGACGCCCGAGCA
GTGGAAGTCCCACAGAAGCTACAGCTGCCGGGTCACGCATGAAGGGAGCACCGTGGAGAAGACA
GTGGCCCCTGCAGAATGCTCT
>N49P9.4
HEAVY CHAIN
SEQ ID NO: 329

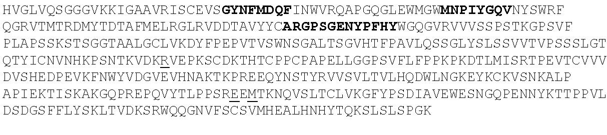

SEQ ID NO: 330
CACGTCCAATTGGTGCAGTCTGGAGGTGGGGTGAAGAAGATTGGGGCCGCTGTGAGGATCTCCTG
CGAGGTGTCTGGATACAACTTCATGGACCAATTCATAAATTGGGTGCGACAGGCCCCCGGTCAGG
GCCTTGAGTGGATGGGATGGATGAACCCAATATATGGACAAGTAAATTATTCATGGAGATTTCAA
GGAAGGGTCACCATGACCAGGGACATGTACACCGACACGGCCTTCATGGAGTTGAGAGGACTGAG
AGTGGACGACACGGCCGTCTATTATTGCGCGAGGGGACCCTCTGGGGAAAATTATCCTTTTCACTA
TTGGGGCCAGGGTGTCCGAGTGGTCGTCTCGTCACCCTCCACCAAGGGCCCATCGGTCTTCCCCCT
GGCACCCTCCTCCAAGAGCACCTCTGGGGGCACAGCGGCCCTGGGCTGCCTGGTCAAGGACTACT
TCCCCGAACCGGTGACGGTGTCGTGGAACTCAGGCGCCCTGACCAGCGGCGTGCACACCTTCCCG
GCTGTCCTACAGTCCTCAGGACTCTACTCCCTCAGCAGCGTGGTGACCGTGCCCTCCAGCAGCTTG
GGCACCCAGACCTACATCTGCAACGTGAATCACAAGCCCAGCAACACCAAGGTGGACAAGAGAG
TTGAGCCCAAATCTTGTGACAAAACTCACACATGCCCACCGTGCCCAGCACCTGAACTCCTGGGG
GGACCGTCAGTCTTCCTCTTCCCCCCAAAACCCAAGGACACCCTCATGATCTCCCGGACCCCTGAG
GTCACATGCGTGGTGGTGGACGTGAGCCACGAAGACCCTGAGGTCAAGTTCAACTGGTACGTGGA
CGGCGTGGAGGTGCATAATGCCAAGACAAAGCCGCGGGAGGAGCAGTACAACAGCACGTACCGG
GTGGTCAGCGTCCTCACCGTCCTGCACCAGGACTGGCTGAATGGCAAGGAGTACAAGTGCAAGGT
CTCCAACAAAGCCCTCCCAGCCCCCATCGAGAAAACCATCTCCAAAGCCAAAGGGCAGCCCCGAG
AACCACAGGTGTACACCCTGCCCCCATCCCGGGAGGAGATGACCAAGAACCAGGTCAGCCTGACC
TGCCTGGTCAAAGGCTTCTATCCCAGCGACATCGCCGTGGAGTGGGAGAGCAATGGGCAGCCGGA
GAACAACTACAAGACCACGCCTCCCGTGCTGGACTCCGACGGCTCCTTCTTCCTCTACAGCAAGCT
CACCGTGGACAAGAGCAGGTGGCAGCAGGGGAACGTCTTCTCATGCTCCGTGATGCATGAGGCTC
TGCACAACCACTACACGCAGAAGAGCCTCTCCCTGTCTCCGGGTAAA
LIGHT CHAIN
SEQ ID NO: 331

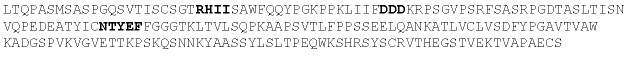

SEQ ID NO: 332
CTGACTCAGCCTGCCTCCATGTCTGCGTCCCCTGGACAGTCGGTAACCATCTCGTGCTCTGGAACC
AGACACATAATCTCTGCTTGGTTCCAACAATATCCAGGCAAACCACCCAAACTCATAATTTTTGAC
GACGATAAGCGTCCCTCTGGAGTTCCTAGTCGCTTCTCTGCCTCCAGGCCTGGCGACACGGCCTCC
CTGACAATCTCTAATGTTCAACCTGAGGACGAGGCGACATACATTTGCAATACATATGAATTCTTT
GGCGGAGGGACCAAATTGACCGTCCTAAGTCAGCCCAAGGCTGCCCCCTCGGTCACTCTGTTCCC
ACCCTCCTCTGAGGAGCTTCAAGCCAACAAGGCCACACTGGTGTGTCTCGTAAGTGACTTCTACCC
GGGAGCCGTGACAGTGGCCTGGAAGGCAGATGGCAGCCCCGTCAAGGTGGGAGTGGAGACCACC
AAACCCTCCAAACAAAGCAACAACAAGTATGCGGCCAGCAGCTACCTGAGCCTGACGCCCGAGC
AGTGGAAGTCCCACAGAAGCTACAGCTGCCGGGTCACGCATGAAGGGAGCACCGTGGAGAAGAC
AGTGGCCCCTGCAGAATGCTCT
>N49P51
HEAVY CHAIN
SEQ ID NO: 333

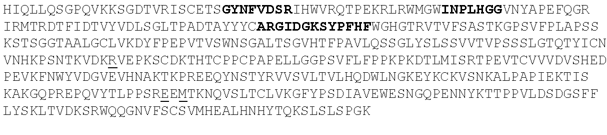

SEQ ID NO: 334
CACATTCAGTTGCTACAGTCGGGGCCTCAAGTTAAGAAGTCTGGGGACACAGTGAGAATCTCCTG
TGAGACCTCTGGATATAACTTCGTCGACTCCCGTATCCACTGGGTCCGACAGACCCCGGAAAAAC
GTCTCAGATGGATGGGCTGGATCAATCCTCTCCACGGTGGTGTGAATTACGCGCCGGAATTTCAGG
GCAGAATCAGGATGACCAGGGACACATTTATAGACACAGTTTACGTGGACCTGAGCGGACTGACA
CCGGCCGACACGGCCTATTATTACTGCGCGCGAGGGATCGATGGCAAGTCTTACCCCTTTCATTTC
TGGGGCCACGGAACCCGGGTCACCGTCTTCTCGGCCTCCACCAAGGGCCCATCGGTCTTCCCCCTG
GCACCCTCCTCCAAGAGCACCTCTGGGGGCACAGCGGCCCTGGGCTGCCTGGTCAAGGACTACTT
CCCCGAACCGGTGACGGTGTCGTGGAACTCAGGCGCCCTGACCAGCGGCGTGCACACCTTCCCGG
CTGTCCTACAGTCCTCAGGACTCTACTCCCTCAGCAGCGTGGTGACCGTGCCCTCCAGCAGCTTGG
GCACCCAGACCTACATCTGCAACGTGAATCACAAGCCCAGCAACACCAAGGTGGACAAGAGAGTT
GAGCCCAAATCTTGTGACAAAACTCACACATGCCCACCGTGCCCAGCACCTGAACTCCTGGGGGG
ACCGTCAGTCTTCCTCTTCCCCCCAAAACCCAAGGACACCCTCATGATCTCCCGGACCCCTGAGGT
CACATGCGTGGTGGTGGACGTGAGCCACGAAGACCCTGAGGTCAAGTTCAACTGGTACGTGGACG
GCGTGGAGGTGCATAATGCCAAGACAAAGCCGCGGGAGGAGCAGTACAACAGCACGTACCGGGT
GGTCAGCGTCCTCACCGTCCTGCACCAGGACTGGCTGAATGGCAAGGAGTACAAGTGCAAGGTCT
CCAACAAAGCCCTCCCAGCCCCCATCGAGAAAACCATCTCCAAAGCCAAAGGGCAGCCCCGAGA
ACCACAGGTGTACACCCTGCCCCCATCCCGGGAGGAGATGACCAAGAACCAGGTCAGCCTGACCT
GCCTGGTCAAAGGCTTCTATCCCAGCGACATCGCCGTGGAGTGGGAGAGCAATGGGCAGCCGGAG
AACAACTACAAGACCACGCCTCCCGTGCTGGACTCCGACGGCTCCTTCTTCCTCTACAGCAAGCTC
ACCGTGGACAAGAGCAGGTGGCAGCAGGGGAACGTCTTCTCATGCTCCGTGATGCATGAGGCTCT
GCACAACCACTACACGCAGAAGAGCCTCTCCCTGTCTCCGGGTAAA
LIGHT CHAIN
SEQ ID NO: 335

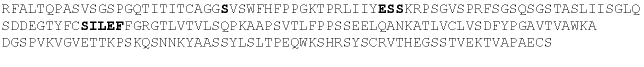

SEQ ID NO: 336
CGATTTGCCCTGACTCAACCTGCCTCCGTGTCTGGGTCTCCTGGACAGACGATCACCATAACCTGC
GCTGGAGGCAGCGTCTCCTGGTTTCATTTCCCTCCAGGCAAAACCCCCAGACTCATTATTTATGAG
TCTTCTAAGCGACCCTCTGGGGTCTCTCCTCGATTCTCTGGGTCCCAGTCTGGCAGCACGGCCTCCC
TCATAATTTCTGGCCTCCAGTCTGATGACGAAGGGACATACTTCTGTTCTATTCTTGAATTTTTCGG
CAGAGGGACTCTTGTCACCGTCCTGAGTCAGCCCAAGGCTGCCCCCTCGGTCACTCTGTTCCCACC
CTCCTCTGAGGAGCTTCAAGCCAACAAGGCCACACTGGTGTGTCTCGTAAGTGACTTCTACCCGGG
AGCCGTGACAGTGGCCTGGAAGGCAGATGGCAGCCCCGTCAAGGTGGGAGTGGAGACCACCAAA
CCCTCCAAACAAAGCAACAACAAGTATGCGGCCAGCAGCTACCTGAGCCTGACGCCCGAGCAGTG
GAAGTCCCACAGAAGCTACAGCTGCCGGGTCACGCATGAAGGGAGCACCGTGGAGAAGACAGTG
GCCCCTGCAGAATGCTCT
>N49P52
HEAVY CHAIN
SEQ ID NO: 337

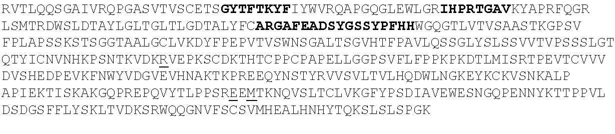

SEQ ID NO: 338
CGTGTTACATTACAACAATCTGGGGCTATAGTGAGGCAGCCTGGGGCCTCAGTGACCGTCTCCTGC
GAGACTTCTGGATATACTTTCACCAAGTATTTCATCTACTGGGTGCGACAGGCCCCTGGACAGGGT
CTTGAGTGGCTGGGCAGAATACACCCCCGAACCGGTGCCGTGAAGTATGCACCGAGATTTCAGGG
TAGACTGTCCATGACCAGAGACTGGTCACTCGACACAGCCTACCTCGGATTGACCGGACTGACAC
TCGGCGACACGGCTCTATATTTCTGTGCGAGGGGGGCCTTTGAGGCAGATTCATATGGGTCAAGTT
ATCCCTTTCACCACTGGGGCCAGGGAACCCTAGTCACCGTCTCCGCGGCCTCCACCAAGGGCCCAT
CGGTCTTCCCCCTGGCACCCTCCTCCAAGAGCACCTCTGGGGGCACAGCGGCCCTGGGCTGCCTGG
TCAAGGACTACTTCCCCGAACCGGTGACGGTGTCGTGGAACTCAGGCGCCCTGACCAGCGGCGTG
CACACCTTCCCGGCTGTCCTACAGTCCTCAGGACTCTACTCCCTCAGCAGCGTGGTGACCGTGCCC
TCCAGCAGCTTGGGCACCCAGACCTACATCTGCAACGTGAATCACAAGCCCAGCAACACCAAGGT
GGACAAGAGAGTTGAGCCCAAATCTTGTGACAAAACTCACACATGCCCACCGTGCCCAGCACCTG
AACTCCTGGGGGGACCGTCAGTCTTCCTCTTCCCCCCAAAACCCAAGGACACCCTCATGATCTCCC
GGACCCCTGAGGTCACATGCGTGGTGGTGGACGTGAGCCACGAAGACCCTGAGGTCAAGTTCAAC
TGGTACGTGGACGGCGTGGAGGTGCATAATGCCAAGACAAAGCCGCGGGAGGAGCAGTACAACA
GCACGTACCGGGTGGTCAGCGTCCTCACCGTCCTGCACCAGGACTGGCTGAATGGCAAGGAGTAC
AAGTGCAAGGTCTCCAACAAAGCCCTCCCAGCCCCCATCGAGAAAACCATCTCCAAAGCCAAAGG
GCAGCCCCGAGAACCACAGGTGTACACCCTGCCCCCATCCCGGGAGGAGATGACCAAGAACCAG
GTCAGCCTGACCTGCCTGGTCAAAGGCTTCTATCCCAGCGACATCGCCGTGGAGTGGGAGAGCAA
TGGGCAGCCGGAGAACAACTACAAGACCACGCCTCCCGTGCTGGACTCCGACGGCTCCTTCTTCCT
CTACAGCAAGCTCACCGTGGACAAGAGCAGGTGGCAGCAGGGGAACGTCTTCTCATGCTCCGTGA
TGCATGAGGCTCTGCACAACCACTACACGCAGAAGAGCCTCTCCCTGTCTCCGGGTAAA
LIGHT CHAIN
SEQ ID NO: 339

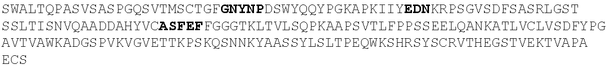

SEQ ID NO: 340
TCCTGGGCCCTGACTCAACCCGCCTCCGTGTCTGCGTCTCCTGGGCAGTCGGTCACCATGTCCTGC
ACTGGATTCGGAAATTATAACCCTGACTCCTGGTACCAACAATACCCAGGCAAAGCCCCCAAACT
CATCATTTATGAAGACAATAAAAGACCCTCGGGGGTCTCTGATCGCTTCTCTGCCTCCAGACTTGG
CAGCACGTCTTCCCTGACAATCTCTAACGTCCAGGCTGCGGACGACGCCCATTATGTCTGCGCCTC
CTTTGAATTTTTCGGCGGAGGGACCAAGCTGACCGTCCTGAGTCAGCCCAAGGCTGCCCCCTCGGT
CACTCTGTTCCCACCCTCCTCTGAGGAGCTTCAAGCCAACAAGGCCACACTGGTGTGTCTCGTAAG
TGACTTCTACCCGGGAGCCGTGACAGTGGCCTGGAAGGCAGATGGCAGCCCCGTCAAGGTGGGAG
TGGAGACCACCAAACCCTCCAAACAAAGCAACAACAAGTATGCGGCCAGCAGCTACCTGAGCCTG
ACGCCCGAGCAGTGGAAGTCCCACAGAAGCTACAGCTGCCGGGTCACGCATGAAGGGAGCACCG
TGGAGAAGACAGTGGCCCCTGCAGAATGCTCT
>N49P53
HEAVY CHAIN
SEQ ID NO: 341

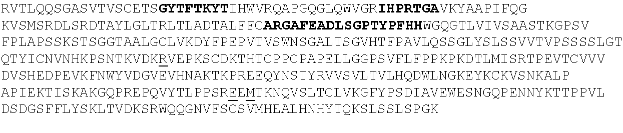

SEQ ID NO: 342
CGTGTGACATTACAACAATCTGGGGCTACAGTGAAGCAGCCTGGGGCCTCAGTGACCGTCTCCTG
CGAGACTTCTGGATACACTTTCACCAAGTATACCATTCACTGGGTGCGACAGGCCCCTGGACAGG
GTCTTCAGTGGGTGGGCAGAATACACCCCCGAACCGGTGCCGTGAAGTATGCACCGATATTTCAG
GGTAAAGTGTCCATGAGTCGAGACTTGTCACGCGACACAGCCTACCTCGGATTGACCAGACTGAC
GCTCGCCGACACGGCTCTATTTTTCTGTGCGAGGGGGGCCTTTGAGGCAGATTTAAGTGGGCCAAC
TTACCCCTTTCACCACTGGGGCCAAGGAACCCTAGTCATCGTCTCCGCGGCCTCCACCAAGGGCCC
ATCGGTCTTCCCCCTGGCACCCTCCTCCAAGAGCACCTCTGGGGGCACAGCGGCCCTGGGCTGCCT
GGTCAAGGACTACTTCCCCGAACCGGTGACGGTGTCGTGGAACTCAGGCGCCCTGACCAGCGGCG
TGCACACCTTCCCGGCTGTCCTACAGTCCTCAGGACTCTACTCCCTCAGCAGCGTGGTGACCGTGC
CCTCCAGCAGCTTGGGCACCCAGACCTACATCTGCAACGTGAATCACAAGCCCAGCAACACCAAG
GTGGACAAGAGAGTTGAGCCCAAATCTTGTGACAAAACTCACACATGCCCACCGTGCCCAGCACC
TGAACTCCTGGGGGGACCGTCAGTCTTCCTCTTCCCCCCAAAACCCAAGGACACCCTCATGATCTC
CCGGACCCCTGAGGTCACATGCGTGGTGGTGGACGTGAGCCACGAAGACCCTGAGGTCAAGTTCA
ACTGGTACGTGGACGGCGTGGAGGTGCATAATGCCAAGACAAAGCCGCGGGAGGAGCAGTACAA
CAGCACGTACCGGGTGGTCAGCGTCCTCACCGTCCTGCACCAGGACTGGCTGAATGGCAAGGAGT
ACAAGTGCAAGGTCTCCAACAAAGCCCTCCCAGCCCCCATCGAGAAAACCATCTCCAAAGCCAAA
GGGCAGCCCCGAGAACCACAGGTGTACACCCTGCCCCCATCCCGGGAGGAGATGACCAAGAACC
AGGTCAGCCTGACCTGCCTGGTCAAAGGCTTCTATCCCAGCGACATCGCCGTGGAGTGGGAGAGC
AATGGGCAGCCGGAGAACAACTACAAGACCACGCCTCCCGTGCTGGACTCCGACGGCTCCTTCTT
CCTCTACAGCAAGCTCACCGTGGACAAGAGCAGGTGGCAGCAGGGGAACGTCTTCTCATGCTCCG
TGATGCATGAGGCTCTGCACAACCACTACACGCAGAAGAGCCTCTCCCTGTCTCCGGGTAAA
LIGHT CHAIN
SEQ ID NO: 343

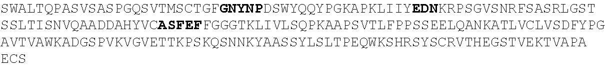

SEQ ID NO: 344
TCCTGGGCCCTGACTCAACCCGCCTCCGTGTCTGCGTCTCCTGGGCAGTCGGTCACCATGTCCTGC
ACTGGATTCGGAAATTATAACCCTGACTCCTGGTACCAACAATACCCAGGCAAAGCCCCCAAACT
CATCATTTATGAGGACAATAAAAGACCCTCGGGAGTCTCTAATCGCTTCTCTGCCTCCAGACTTGG
CAGCACGTCTTCCCTGACAATCTCTAACGTCCAGGCCGCTGACGACGCCCATTATGTCTGCGCCTC
CTTTGAATTTTTCGGCGGAGGGACCAAGCTGATCGTCCTGAGTCAGCCCAAGGCTGCCCCCTCGGT
CACTCTGTTCCCACCCTCCTCTGAGGAGCTTCAAGCCAACAAGGCCACACTGGTGTGTCTCGTAAG
TGACTTCTACCCGGGAGCCGTGACAGTGGCCTGGAAGGCAGATGGCAGCCCCGTCAAGGTGGGAG
TGGAGACCACCAAACCCTCCAAACAAAGCAACAACAAGTATGCGGCCAGCAGCTACCTGAGCCTG
ACGCCCGAGCAGTGGAAGTCCCACAGAAGCTACAGCTGCCGGGTCACGCATGAAGGGAGCACCG
TGGAGAAGACAGTGGCCCCTGCAGAATGCTCT
>N49P54
HEAVY CHAIN
SEQ ID NO: 345

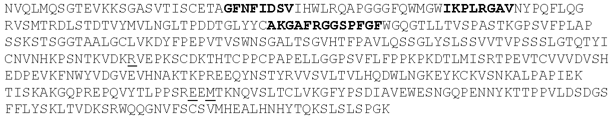

SEQ ID NO: 346
AACGTGCAGTTGATGCAGTCTGGGACTGAGGTGAAGAAGTCTGGGGCCTCGGTGACAATCTCTTG
TGAGACCGCTGGATTCAACTTCATCGACTCCGTCATACACTGGCTGCGCCAGGCCCCTGGAGGAG
GATTTCAGTGGATGGGGTGGATCAAGCCTCTTAGAGGTGCCGTCAATTATCCACAGTTTTTGCAGG
GCAGGGTCTCCATGACCCGGGACTTGTCCACCGACACGGTGTACATGGTCTTGAATGGACTGACA
CCTGACGACACAGGCCTTTATTACTGCGCGAAAGGGGCCTTTAGAGGGGGTTCTCCCTTTGGCTTC
TGGGGCCAGGGAACTCTGCTCACCGTCTCCCCAGCCTCCACCAAGGGCCCATCGGTCTTCCCCCTG
GCACCCTCCTCCAAGAGCACCTCTGGGGGCACAGCGGCCCTGGGCTGCCTGGTCAAGGACTACTT
CCCCGAACCGGTGACGGTGTCGTGGAACTCAGGCGCCCTGACCAGCGGCGTGCACACCTTCCCGG
CTGTCCTACAGTCCTCAGGACTCTACTCCCTCAGCAGCGTGGTGACCGTGCCCTCCAGCAGCTTGG
GCACCCAGACCTACATCTGCAACGTGAATCACAAGCCCAGCAACACCAAGGTGGACAAGAGAGTT
GAGCCCAAATCTTGTGACAAAACTCACACATGCCCACCGTGCCCAGCACCTGAACTCCTGGGGGG
ACCGTCAGTCTTCCTCTTCCCCCCAAAACCCAAGGACACCCTCATGATCTCCCGGACCCCTGAGGT
CACATGCGTGGTGGTGGACGTGAGCCACGAAGACCCTGAGGTCAAGTTCAACTGGTACGTGGACG
GCGTGGAGGTGCATAATGCCAAGACAAAGCCGCGGGAGGAGCAGTACAACAGCACGTACCGGGT
GGTCAGCGTCCTCACCGTCCTGCACCAGGACTGGCTGAATGGCAAGGAGTACAAGTGCAAGGTCT
CCAACAAAGCCCTCCCAGCCCCCATCGAGAAAACCATCTCCAAAGCCAAAGGGCAGCCCCGAGA
ACCACAGGTGTACACCCTGCCCCCATCCCGGGAGGAGATGACCAAGAACCAGGTCAGCCTGACCT
GCCTGGTCAAAGGCTTCTATCCCAGCGACATCGCCGTGGAGTGGGAGAGCAATGGGCAGCCGGAG
AACAACTACAAGACCACGCCTCCCGTGCTGGACTCCGACGGCTCCTTCTTCCTCTACAGCAAGCTC
ACCGTGGACAAGAGCAGGTGGCAGCAGGGGAACGTCTTCTCATGCTCCGTGATGCATGAGGCTCT
GCACAACCACTACACGCAGAAGAGCCTCTCCCTGTCTCCGGGTAAA
LIGHT CHAIN
SEQ ID NO: 347

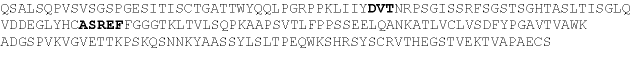

SEQ ID NO: 348
CAGTCTGCCCTGTCTCAGCCTGTCTCCGTGTCTGGGTCTCCTGGAGAGTCGATCACCATTTCCTGTA
CTGGAGCCACCACCTGGTATCAACAACTCCCAGGCAGACCCCCCAAACTCATCATTTATGACGTCA
CTAACCGGCCCTCAGGCATTTCTAGTCGTTTCTCTGGCTCCACGTCTGGCCACACGGCCTCCCTGA
CAATCTCCGGTCTCCAGGTTGACGACGAGGGTCTGTATCACTGCGCCTCACGTGAATTTTTCGGCG
GAGGGACCAAGCTGACCGTCCTGAGTCAGCCCAAGGCTGCCCCCTCGGTCACTCTGTTCCCACCCT
CCTCTGAGGAGCTTCAAGCCAACAAGGCCACACTGGTGTGTCTCGTAAGTGACTTCTACCCGGGA
GCCGTGACAGTGGCCTGGAAGGCAGATGGCAGCCCCGTCAAGGTGGGAGTGGAGACCACCAAAC
CCTCCAAACAAAGCAACAACAAGTATGCGGCCAGCAGCTACCTGAGCCTGACGCCCGAGCAGTGG
AAGTCCCACAGAAGCTACAGCTGCCGGGTCACGCATGAAGGGAGCACCGTGGAGAAGACAGTGG
CCCCTGCAGAATGCTCT
>N49P60
HEAVY CHAIN
SEQ ID NO: 349

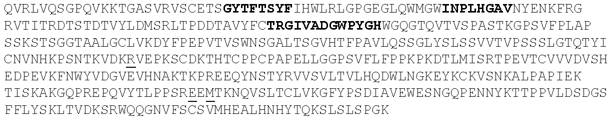

SEQ ID NO: 350
CAGGTGCGACTGGTGCAGTCTGGGCCTCAGGTGAAGAAGACTGGGGCCTCAGTGAGGGTCTCCTG
CGAAACCTCTGGATACACGTTCACCTCCTACTTCATCCATTGGTTACGACTGGGCCCCGGAGAGGG
GCTTCAGTGGATGGGGTGGATCAACCCTTTACATGGTGCCGTGAATTATGAAAACAAATTTAGGG
GCAGGGTCACAATCACCAGGGACACGTCCACAGACACAGTGTATTTGGACATGAGCAGACTGACC
CCTGACGACACGGCCGTCTATTTCTGCACAAGAGGAATCGTTGCTGATGGGTGGCCCTATGGCCAC
TGGGGCCAGGGAACCCAAGTCACCGTCTCCCCGGCCTCCACCAAGGGCCCATCGGTCTTCCCCCTG
GCACCCTCCTCCAAGAGCACCTCTGGGGGCACAGCGGCCCTGGGCTGCCTGGTCAAGGACTACTT
CCCCGAACCGGTGACGGTGTCGTGGAACTCAGGCGCCCTGACCAGCGGCGTGCACACCTTCCCGG
CTGTCCTACAGTCCTCAGGACTCTACTCCCTCAGCAGCGTGGTGACCGTGCCCTCCAGCAGCTTGG
GCACCCAGACCTACATCTGCAACGTGAATCACAAGCCCAGCAACACCAAGGTGGACAAGAGAGTT
GAGCCCAAATCTTGTGACAAAACTCACACATGCCCACCGTGCCCAGCACCTGAACTCCTGGGGGG
ACCGTCAGTCTTCCTCTTCCCCCCAAAACCCAAGGACACCCTCATGATCTCCCGGACCCCTGAGGT
CACATGCGTGGTGGTGGACGTGAGCCACGAAGACCCTGAGGTCAAGTTCAACTGGTACGTGGACG
GCGTGGAGGTGCATAATGCCAAGACAAAGCCGCGGGAGGAGCAGTACAACAGCACGTACCGGGT
GGTCAGCGTCCTCACCGTCCTGCACCAGGACTGGCTGAATGGCAAGGAGTACAAGTGCAAGGTCT
CCAACAAAGCCCTCCCAGCCCCCATCGAGAAAACCATCTCCAAAGCCAAAGGGCAGCCCCGAGA
ACCACAGGTGTACACCCTGCCCCCATCCCGGGAGGAGATGACCAAGAACCAGGTCAGCCTGACCT
GCCTGGTCAAAGGCTTCTATCCCAGCGACATCGCCGTGGAGTGGGAGAGCAATGGGCAGCCGGAG
AACAACTACAAGACCACGCCTCCCGTGCTGGACTCCGACGGCTCCTTCTTCCTCTACAGCAAGCTC
ACCGTGGACAAGAGCAGGTGGCAGCAGGGGAACGTCTTCTCATGCTCCGTGATGCATGAGGCTCT
GCACAACCACTACACGCAGAAGAGCCTCTCCCTGTCTCCGGGTAAA
LIGHT CHAIN
SEQ ID NO: 351

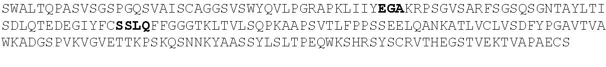

SEQ ID NO: 352
TCCTGGGCCCTGACTCAGCCTGCCTCCGTGTCTGGGTCTCCTGGACAGTCGGTCGCCATCTCCTGC
GCTGGCGGCAGCGTCTCCTGGTACCAGGTGCTCCCAGGCAGAGCCCCCAAACTCATCATTTATGA
GGGCGCTAAGCGACCCTCAGGGGTTTCTGCTCGCTTCTCTGGCTCCCAGTCTGGCAACACGGCTTA
CCTGACAATTTCTGACCTCCAGACTGAGGACGAGGGCATCTACTTCTGCTCTTCACTTCAATTCTTC
GGCGGAGGGACCAAACTGACCGTCCTAAGTCAGCCCAAGGCTGCCCCCTCGGTCACTCTGTTCCC
ACCCTCCTCTGAGGAGCTTCAAGCCAACAAGGCCACACTGGTGTGTCTCGTAAGTGACTTCTACCC
GGGAGCCGTGACAGTGGCCTGGAAGGCAGATGGCAGCCCCGTCAAGGTGGGAGTGGAGACCACC
AAACCCTCCAAACAAAGCAACAACAAGTATGCGGCCAGCAGCTACCTGAGCCTGACGCCCGAGC
AGTGGAAGTCCCACAGAAGCTACAGCTGCCGGGTCACGCATGAAGGGAGCACCGTGGAGAAGAC
AGTGGCCCCTGCAGAATGCTCT
>N49P61
HEAVY CHAIN
SEQ ID NO: 353

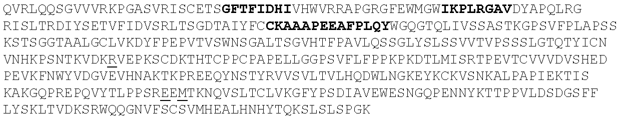

SEQ ID NO: 354
CAGGTGCGACTTCAGCAGTCTGGTGTCGTGGTGAGGAAGCCTGGGGCCTCAGTGAGAATTTCCTG
CGAGACTTCTGGATTCACCTTCATCGACCACATTGTCCATTGGGTGCGGCGGGCCCCTGGACGAGG
CTTTGAATGGATGGGTTGGATCAAGCCTCTTAGGGGTGCCGTAGATTATGCACCCCAACTTCGGGG
CAGGATCTCCCTGACGAGGGACATTTACAGTGAAACCGTCTTTATAGACGTGAGCCGACTGACGT
CTGGCGACACGGCGATATACTTTTGTTGTAAGGCCGCCGCCCCTGAAGAAGCATTCCCCCTTCAAT
ACTGGGGCCAGGGGACCCAACTTATCGTCTCCTCGGCCTCCACCAAGGGCCCATCGGTCTITCCCCC
TGGCACCCTCCTCCAAGAGCACCTCTGGGGGCACAGCGGCCCTGGGCTGCCTGGTCAAGGACTAC
TTCCCCGAACCGGTGACGGTGTCGTGGAACTCAGGCGCCCTGACCAGCGGCGTGCACACCTTCCC
GGCTGTCCTACAGTCCTCAGGACTCTACTCCCTCAGCAGCGTGGTGACCGTGCCCTCCAGCAGCTT
GGGCACCCAGACCTACATCTGCAACGTGAATCACAAGCCCAGCAACACCAAGGTGGACAAGAGA
GTTGAGCCCAAATCTTGTGACAAAACTCACACATGCCCACCGTGCCCAGCACCTGAACTCCTGGG
GGGACCGTCAGTCTTCCTCTTCCCCCCAAAACCCAAGGACACCCTCATGATCTCCCGGACCCCTGA
GGTCACATGCGTGGTGGTGGACGTGAGCCACGAAGACCCTGAGGTCAAGTTCAACTGGTACGTGG
ACGGCGTGGAGGTGCATAATGCCAAGACAAAGCCGCGGGAGGAGCAGTACAACAGCACGTACCG
GGTGGTCAGCGTCCTCACCGTCCTGCACCAGGACTGGCTGAATGGCAAGGAGTACAAGTGCAAGG
TCTCCAACAAAGCCCTCCCAGCCCCCATCGAGAAAACCATCTCCAAAGCCAAAGGGCAGCCCCGA
GAACCACAGGTGTACACCCTGCCCCCATCCCGGGAGGAGATGACCAAGAACCAGGTCAGCCTGAC
CTGCCTGGTCAAAGGCTTCTATCCCAGCGACATCGCCGTGGAGTGGGAGAGCAATGGGCAGCCGG
AGAACAACTACAAGACCACGCCTCCCGTGCTGGACTCCGACGGCTCCTTCTTCCTCTACAGCAAGC
TCACCGTGGACAAGAGCAGGTGGCAGCAGGGGAACGTCTTCTCATGCTCCGTGATGCATGAGGCT
CTGCACAACCACTACACGCAGAAGAGCCTCTCCCTGTCTCCGGGTAAA
LIGHT CHAIN
SEQ ID NO: 355

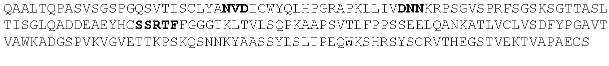

SEQ ID NO: 356
CAGGCTGCCCTGACTCAGCCCGCCTCCGTGTCCGGCTCTCCTGGACAGTCGGTCACCATTTCCTGC
CTTTATGCCAATGTAGATATCTGCTGGTATCAACTACACCCGGGCAGAGCCCCCAAACTTCTAATT
GTTGACAATAATAAGCGGCCCTCAGGAGTCTCTCCTCGCTTCTCTGGCTCCAAGTCTGGCACCACG
GCCTCCCTGACAATCTCTGGACTTCAGGCTGACGACGAGGCTGAATATCACTGCTCTTCAAGAACA
TTTTTTGGCGGGGGGACCAAGTTGACCGTCCTGAGTCAGCCCAAGGCTGCCCCCTCGGTCACTCTG
TTCCCACCCTCCTCTGAGGAGCTTCAAGCCAACAAGGCCACACTGGTGTGTCTCGTAAGTGACTTC
TACCCGGGAGCCGTGACAGTGGCCTGGAAGGCAGATGGCAGCCCCGTCAAGGTGGGAGTGGAGA
CCACCAAACCCTCCAAACAAAGCAACAACAAGTATGCGGCCAGCAGCTACCTGAGCCTGACGCCC
GAGCAGTGGAAGTCCCACAGAAGCTACAGCTGCCGGGTCACGCATGAAGGGAGCACCGTGGAGA
AGACAGTGGCCCCTGCAGAATGCTCT
>N49P62
HEAVY CHAIN
SEQ ID NO: 357

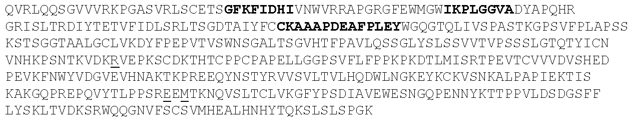

SEQ ID NO: 358
CAGGTGCGACTTCAGCAGTCTGGTGTCGTGGTGAGGAAGCCTGGGGCCTCAGTGAGACTTTCCTGC
GAGACGTCTGGATTCAAATTCATCGACCACATTGTCAACTGGGTGCGGCGGGCCCCTGGACGAGG
CTTTGAATGGATGGGTTGGATCAAGCCTCTTGGGGGTGTCGCTGATTATGCACCCCAACATCGGGG
CAGGATCTCACTGACGAGGGACATTTACACTGAAACCGTCTTTATAGACCTGAGTCGACTGACGTC
TGGCGACACGGCGATTTATTTCTGTTGTAAGGCCGCCGCCCCTGATGAAGCATTCCCCCTTGAATA
CTGGGGCCAGGGGACCCAACTTATCGTCTCCCCGGCCTCCACCAAGGGCCCATCGGTCITTCCCCCT
GGCACCCTCCTCCAAGAGCACCTCTGGGGGCACAGCGGCCCTGGGCTGCCTGGTCAAGGACTACT
TCCCCGAACCGGTGACGGTGTCGTGGAACTCAGGCGCCCTGACCAGCGGCGTGCACACCITTCCCG
GCTGTCCTACAGTCCTCAGGACTCTACTCCCTCAGCAGCGTGGTGACCGTGCCCTCCAGCAGCTTG
GGCACCCAGACCTACATCTGCAACGTGAATCACAAGCCCAGCAACACCAAGGTGGACAAGAGAG
TTGAGCCCAAATCTTGTGACAAAACTCACACATGCCCACCGTGCCCAGCACCTGAACTCCTGGGG
GGACCGTCAGTCTTCCTCTTCCCCCCAAAACCCAAGGACACCCTCATGATCTCCCGGACCCCTGAG
GTCACATGCGTGGTGGTGGACGTGAGCCACGAAGACCCTGAGGTCAAGTTCAACTGGTACGTGGA
CGGCGTGGAGGTGCATAATGCCAAGACAAAGCCGCGGGAGGAGCAGTACAACAGCACGTACCGG
GTGGTCAGCGTCCTCACCGTCCTGCACCAGGACTGGCTGAATGGCAAGGAGTACAAGTGCAAGGT
CTCCAACAAAGCCCTCCCAGCCCCCATCGAGAAAACCATCTCCAAAGCCAAAGGGCAGCCCCGAG
AACCACAGGTGTACACCCTGCCCCCATCCCGGGAGGAGATGACCAAGAACCAGGTCAGCCTGACC
TGCCTGGTCAAAGGCTTCTATCCCAGCGACATCGCCGTGGAGTGGGAGAGCAATGGGCAGCCGGA
GAACAACTACAAGACCACGCCTCCCGTGCTGGACTCCGACGGCTCCTTCTTCCTCTACAGCAAGCT
CACCGTGGACAAGAGCAGGTGGCAGCAGGGGAACGTCTITCTCATGCTCCGTGATGCATGAGGCTC
TGCACAACCACTACACGCAGAAGAGCCTCTCCCTGTCTCCGGGTAAA
LIGHT CHAIN
SEQ ID NO: 359

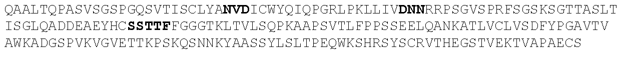

SEQ ID NO: 360
CAGGCTGCCCTGACTCAGCCCGCCTCCGTGTCCGGCTCTCCTGGACAGTCGGTCACCATTTCCTGC
CTTTATGCCAATGTAGATATCTGCTGGTATCAAATACAGCCGGGCAGATTACCCAAACTTCTGATT
GTTGACAATAATAGGCGACCCTCAGGAGTCTCTCCTCGCTTCTCTGGCTCCAAGTCTGGCACCACG
GCCTCCCTGACAATCTCTGGACTTCAGGCTGACGACGAGGCTGAATATCACTGCTCTTCAACAACA
TTTTTTGGCGGGGGGACCAAGTTGACCGTCCTCAGTCAGCCCAAGGCTGCCCCCTCGGTCACTCTG
TTCCCACCCTCCTCTGAGGAGCTTCAAGCCAACAAGGCCACACTGGTGTGTCTCGTAAGTGACTTC
TACCCGGGAGCCGTGACAGTGGCCTGGAAGGCAGATGGCAGCCCCGTCAAGGTGGGAGTGGAGA
CCACCAAACCCTCCAAACAAAGCAACAACAAGTATGCGGCCAGCAGCTACCTGAGCCTGACGCCC
GAGCAGTGGAAGTCCCACAGAAGCTACAGCTGCCGGGTCACGCATGAAGGGAGCACCGTGGAGA
AGACAGTGGCCCCTGCAGAATGCTCT
>N49P63
HEAVY CHAIN
SEQ ID NO: 361

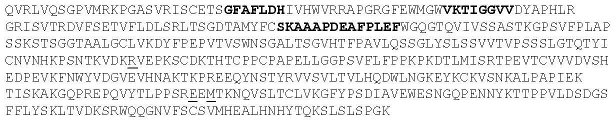

SEQ ID NO: 362
CAGGTGCGACTTGTGCAGTCTGGTCCCGTGATGAGAAAGCCTGGGGCCTCAGTGAGAATTTCTTGC
GAGACATCTGGATTCGCCTTCTTGGACCACATTGTCCACTGGGTGCGGCGGGCCCCTGGACGCGGC
TTTGAATGGATGGGTTGGGTTAAGACCATTGGGGGTGTCGTTGATTATGCACCCCACCTTAGGGGC
AGGATCTCCGTGACGAGAGACGTCTTTAGTGAAACCGTCTTTCTGGACTTGAGCCGACTGACGTCT
GGCGACACGGCGATGTATTTTTGTTCTAAGGCCGCCGCCCCTGACGAAGCCTTCCCCCTTGAATTT
TGGGGCCAGGGGACCCAAGTCATCGTCTCCTCGGCCTCCACCAAGGGCCCATCGGTCTTCCCCCTG
GCACCCTCCTCCAAGAGCACCTCTGGGGGCACAGCGGCCCTGGGCTGCCTGGTCAAGGACTACTT
CCCCGAACCGGTGACGGTGTCGTGGAACTCAGGCGCCCTGACCAGCGGCGTGCACACCTTCCCGG
CTGTCCTACAGTCCTCAGGACTCTACTCCCTCAGCAGCGTGGTGACCGTGCCCTCCAGCAGCTTGG
GCACCCAGACCTACATCTGCAACGTGAATCACAAGCCCAGCAACACCAAGGTGGACAAGAGAGTT
GAGCCCAAATCTTGTGACAAAACTCACACATGCCCACCGTGCCCAGCACCTGAACTCCTGGGGGG
ACCGTCAGTCTTCCTCTTCCCCCCAAAACCCAAGGACACCCTCATGATCTCCCGGACCCCTGAGGT
CACATGCGTGGTGGTGGACGTGAGCCACGAAGACCCTGAGGTCAAGTTCAACTGGTACGTGGACG
GCGTGGAGGTGCATAATGCCAAGACAAAGCCGCGGGAGGAGCAGTACAACAGCACGTACCGGGT
GGTCAGCGTCCTCACCGTCCTGCACCAGGACTGGCTGAATGGCAAGGAGTACAAGTGCAAGGTCT
CCAACAAAGCCCTCCCAGCCCCCATCGAGAAAACCATCTCCAAAGCCAAAGGGCAGCCCCGAGA
ACCACAGGTGTACACCCTGCCCCCATCCCGGGAGGAGATGACCAAGAACCAGGTCAGCCTGACCT
GCCTGGTCAAAGGCTTCTATCCCAGCGACATCGCCGTGGAGTGGGAGAGCAATGGGCAGCCGGAG
AACAACTACAAGACCACGCCTCCCGTGCTGGACTCCGACGGCTCCTTCTTCCTCTACAGCAAGCTC
ACCGTGGACAAGAGCAGGTGGCAGCAGGGGAACGTCTTCTCATGCTCCGTGATGCATGAGGCTCT
GCACAACCACTACACGCAGAAGAGCCTCTCCCTGTCTCCGGGTAAA
LIGHT CHAIN
SEQ ID NO: 363

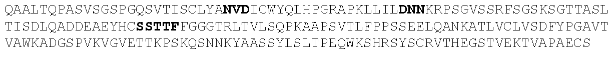

SEQ ID NO: 364
CAGGCTGCCCTGACTCAGCCCGCCTCCGTGTCCGGCTCTCCTGGACAGTCGGTCACCATTTCTTGC
CTTTATGCCAATGTGGATATCTGCTGGTATCAACTTCACCCGGGCAGAGCCCCCAAACTTCTTATT
CTTGACAATAATAAACGGCCCTCAGGAGTCTCTAGTCGCTTCTCCGGTTCCAAGTCTGGCACCACG
GCCTCCCTAACCATCTCTGACCTTCAGGCTGACGACGAGGCTGAATATCACTGCTCTTCAACAACA
TTTTTTGGCGGGGGGACCAGGTTGACCGTCCTGAGTCAGCCCAAGGCTGCCCCCTCGGTCACTCTG
TTCCCACCCTCCTCTGAGGAGCTTCAAGCCAACAAGGCCACACTGGTGTGTCTCGTAAGTGACTTC
TACCCGGGAGCCGTGACAGTGGCCTGGAAGGCAGATGGCAGCCCCGTCAAGGTGGGAGTGGAGA
CCACCAAACCCTCCAAACAAAGCAACAACAAGTATGCGGCCAGCAGCTACCTGAGCCTGACGCCC
GAGCAGTGGAAGTCCCACAGAAGCTACAGCTGCCGGGTCACGCATGAAGGGAGCACCGTGGAGA
AGACAGTGGCCCCTGCAGAATGCTCT
>N49P64
HEAVY CHAIN
SEQ ID NO: 365

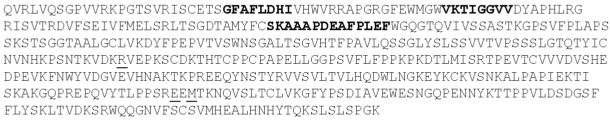

SEQ ID NO: 366
CAGGTGCGACTTGTGCAGTCTGGTCCCGTGGTGAGAAAGCCTGGGACCTCAGTGAGAATTTCTTGC
GAGACATCTGGATTCGCCTTCTTGGACCACATTGTCCACTGGGTGCGGCGGGCCCCTGGACGCGGC
TTTGAATGGATGGGTTGGGTTAAGACCATTGGGGGTGTCGTTGATTATGCACCCCACCTTAGGGGC
AGGATCTCCGTGACGAGGGACGTATTTAGTGAAATCGTCTTTATGGAGTTGAGTCGACTGACGTCT
GGCGACACGGCGATGTATTTTTGTTCTAAGGCCGCCGCCCCTGACGAAGCCTTCCCCCTTGAATTT
TGGGGCCAGGGGACCCAAGTCATCGTCTCCTCGGCCTCCACCAAGGGCCCATCGGTCTTCCCCCTG
GCACCCTCCTCCAAGAGCACCTCTGGGGGCACAGCGGCCCTGGGCTGCCTGGTCAAGGACTACTT
CCCCGAACCGGTGACGGTGTCGTGGAACTCAGGCGCCCTGACCAGCGGCGTGCACACCTTCCCGG
CTGTCCTACAGTCCTCAGGACTCTACTCCCTCAGCAGCGTGGTGACCGTGCCCTCCAGCAGCTTGG
GCACCCAGACCTACATCTGCAACGTGAATCACAAGCCCAGCAACACCAAGGTGGACAAGAGAGTT
GAGCCCAAATCTTGTGACAAAACTCACACATGCCCACCGTGCCCAGCACCTGAACTCCTGGGGGG
ACCGTCAGTCTTCCTCTTCCCCCCAAAACCCAAGGACACCCTCATGATCTCCCGGACCCCTGAGGT
CACATGCGTGGTGGTGGACGTGAGCCACGAAGACCCTGAGGTCAAGTTCAACTGGTACGTGGACG
GCGTGGAGGTGCATAATGCCAAGACAAAGCCGCGGGAGGAGCAGTACAACAGCACGTACCGGGT
GGTCAGCGTCCTCACCGTCCTGCACCAGGACTGGCTGAATGGCAAGGAGTACAAGTGCAAGGTCT
CCAACAAAGCCCTCCCAGCCCCCATCGAGAAAACCATCTCCAAAGCCAAAGGGCAGCCCCGAGA
ACCACAGGTGTACACCCTGCCCCCATCCCGGGAGGAGATGACCAAGAACCAGGTCAGCCTGACCT
GCCTGGTCAAAGGCTTCTATCCCAGCGACATCGCCGTGGAGTGGGAGAGCAATGGGCAGCCGGAG
AACAACTACAAGACCACGCCTCCCGTGCTGGACTCCGACGGCTCCTTCTTCCTCTACAGCAAGCTC
ACCGTGGACAAGAGCAGGTGGCAGCAGGGGAACGTCTTCTCATGCTCCGTGATGCATGAGGCTCT
GCACAACCACTACACGCAGAAGAGCCTCTCCCTGTCTCCGGGTAAA
LIGHT CHAIN
SEQ ID NO: 367

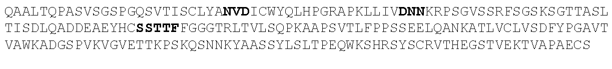

SEQ ID NO: 368
CAGGCTGCCCTGACTCAGCCCGCCTCCGTGTCCGGCTCTCCTGGACAGTCGGTCACCATTTCCTGC
CTTTATGCCAATGTGGATATCTGCTGGTATCAACTTCACCCGGGCAGAGCCCCCAAACTTCTAATT
GTTGACAATAATAAGCGGCCCTCAGGAGTCTCTAGTCGCTTCTCTGGTTCCAAGTCTGGCACCACG
GCCTCCCTAACAATCTCTGATCTTCAGGCTGACGACGAGGCTGAATATCACTGCTCTTCAACAACA
TTTTTTGGCGGGGGGACCAGGTTGACCGTCCTGAGTCAGCCCAAGGCTGCCCCCTCGGTCACTCTG
TTCCCACCCTCCTCTGAGGAGCTTCAAGCCAACAAGGCCACACTGGTGTGTCTCGTAAGTGACTTC
TACCCGGGAGCCGTGACAGTGGCCTGGAAGGCAGATGGCAGCCCCGTCAAGGTGGGAGTGGAGA
CCACCAAACCCTCCAAACAAAGCAACAACAAGTATGCGGCCAGCAGCTACCTGAGCCTGACGCCC
GAGCAGTGGAAGTCCCACAGAAGCTACAGCTGCCGGGTCACGCATGAAGGGAGCACCGTGGAGA
AGACAGTGGCCCCTGCAGAATGCTCT
>N49P65
HEAVY CHAIN
SEQ ID NO: 369

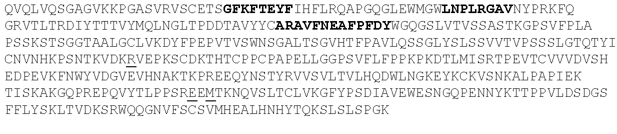

SEQ ID NO: 370
CAAGTGCAACTGGTGCAGTCTGGGGCTGGGGTGAAGAAGCCTGGGGCCTCAGTGAGGGTCTCCTG
CGAGACATCCGGATTCAAGTTCACCGAGTACTTTATCCACTTTTTACGACAGGCCCCTGGACAAGG
GCTTGAGTGGATGGGATGGCTCAACCCTCTCAGAGGTGCCGTCAACTATCCACGGAAGTTTCAGG
GCAGAGTCACTTTGACCAGGGACATCTACACCACCACCGTCTACATGCAACTTAACGGTCTGACCC
CTGACGACACGGCCGTCTACTACTGTGCCAGAGCGGTCTTTAATGAAGCTTTCCCCTTTGACTACT
GGGGCCAGGGAAGCCTGGTCACCGTCTCCTCAGCCTCCACCAAGGGCCCATCGGTCTTCCCCCTGG
CACCCTCCTCCAAGAGCACCTCTGGGGGCACAGCGGCCCTGGGCTGCCTGGTCAAGGACTACTTC
CCCGAACCGGTGACGGTGTCGTGGAACTCAGGCGCCCTGACCAGCGGCGTGCACACCTTCCCGGC
TGTCCTACAGTCCTCAGGACTCTACTCCCTCAGCAGCGTGGTGACCGTGCCCTCCAGCAGCTTGGG
CACCCAGACCTACATCTGCAACGTGAATCACAAGCCCAGCAACACCAAGGTGGACAAGAGAGTTG
AGCCCAAATCTTGTGACAAAACTCACACATGCCCACCGTGCCCAGCACCTGAACTCCTGGGGGGA
CCGTCAGTCTTCCTCTTCCCCCCAAAACCCAAGGACACCCTCATGATCTCCCGGACCCCTGAGGTC
ACATGCGTGGTGGTGGACGTGAGCCACGAAGACCCTGAGGTCAAGTTCAACTGGTACGTGGACGG
CGTGGAGGTGCATAATGCCAAGACAAAGCCGCGGGAGGAGCAGTACAACAGCACGTACCGGGTG
GTCAGCGTCCTCACCGTCCTGCACCAGGACTGGCTGAATGGCAAGGAGTACAAGTGCAAGGTCTC
CAACAAAGCCCTCCCAGCCCCCATCGAGAAAACCATCTCCAAAGCCAAAGGGCAGCCCCGAGAA
CCACAGGTGTACACCCTGCCCCCATCCCGGGAGGAGATGACCAAGAACCAGGTCAGCCTGACCTG
CCTGGTCAAAGGCTTCTATCCCAGCGACATCGCCGTGGAGTGGGAGAGCAATGGGCAGCCGGAGA
ACAACTACAAGACCACGCCTCCCGTGCTGGACTCCGACGGCTCCTTCTTCCTCTACAGCAAGCTCA
CCGTGGACAAGAGCAGGTGGCAGCAGGGGAACGTCTTCTCATGCTCCGTGATGCATGAGGCTCTG
CACAACCACTACACGCAGAAGAGCCTCTCCCTGTCTCCGGGTAAA
LIGHT CHAIN
SEQ ID NO: 371

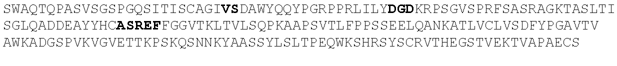

SEQ ID NO: 372
TCCTGGGCCCAGACTCAGCCTGCCTCCGTGTCTGGGTCTCCTGGACAGTCGATCACCATCTCCTGC
GCTGGAATCGTCAGTGATGCCTGGTACCAGCAATACCCAGGCAGACCCCCCAGACTCATCCTTTAT
GACGGCGATAAGCGGCCCTCAGGGGTTITCTCCTCGTTTTTCTGCCTCCAGGGCCGGCAAGACGGCC
TCCCTGACAATTTCTGGGCTGCAGGCTGACGACGAGGCTTATTATCACTGCGCGTCAAGGGAATTT
TTTGGAGGCGTGACCAAGTTGACCGTCCTAAGTCAGCCCAAGGCTGCCCCCTCGGTCACTCTGTTC
CCACCCTCCTCTGAGGAGCTTCAAGCCAACAAGGCCACACTGGTGTGTCTCGTAAGTGACTTCTAC
CCGGGAGCCGTGACAGTGGCCTGGAAGGCAGATGGCAGCCCCGTCAAGGTGGGAGTGGAGACCA
CCAAACCCTCCAAACAAAGCAACAACAAGTATGCGGCCAGCAGCTACCTGAGCCTGACGCCCGAG
CAGTGGAAGTCCCACAGAAGCTACAGCTGCCGGGTCACGCATGAAGGGAGCACCGTGGAGAAGA
CAGTGGCCCCTGCAGAATGCTCT
>N49P66
HEAVY CHAIN
SEQ ID NO: 373

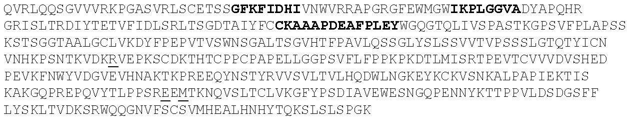

SEQ ID NO: 374
CAGGTGCGACTTCAGCAGTCTGGTGTCGTGGTGAGGAAGCCTGGGGCCTCAGTGAGACTTTCCTGC
GAGACGTCTGGCTTCAAATTCATCGACCACATTGTCAACTGGGTGCGGCGGGCCCCTGGACGAGG
CTTTGAATGGATGGGTTGGATCAAGCCTCTTGGGGGTGTCGCTGATTATGCACCCCAACATCGGGG
CAGGATCTCACTGACGAGGGACATTTACACTGAAACCGTCTTTATAGACCTGAGTCGACTGACGTC
TGGCGACACGGCGATTTATTTTTGTTGTAAGGCCGCCGCCCCTGATGAAGCATTCCCCCTTGAATA
CTGGGGCCAGGGGACCCAACTTATCGTCTCCCCGGCCTCCACCAAGGGCCCATCGGTCTTCCCCCT
GGCACCCTCCTCCAAGAGCACCTCTGGGGGCACAGCGGCCCTGGGCTGCCTGGTCAAGGACTACT
TCCCCGAACCGGTGACGGTGTCGTGGAACTCAGGCGCCCTGACCAGCGGCGTGCACACCTTCCCG
GCTGTCCTACAGTCCTCAGGACTCTACTCCCTCAGCAGCGTGGTGACCGTGCCCTCCAGCAGCTTG
GGCACCCAGACCTACATCTGCAACGTGAATCACAAGCCCAGCAACACCAAGGTGGACAAGAGAG
TTGAGCCCAAATCTTGTGACAAAACTCACACATGCCCACCGTGCCCAGCACCTGAACTCCTGGGG
GGACCGTCAGTCTTCCTCTTCCCCCCAAAACCCAAGGACACCCTCATGATCTCCCGGACCCCTGAG
GTCACATGCGTGGTGGTGGACGTGAGCCACGAAGACCCTGAGGTCAAGTTCAACTGGTACGTGGA
CGGCGTGGAGGTGCATAATGCCAAGACAAAGCCGCGGGAGGAGCAGTACAACAGCACGTACCGG
GTGGTCAGCGTCCTCACCGTCCTGCACCAGGACTGGCTGAATGGCAAGGAGTACAAGTGCAAGGT
CTCCAACAAAGCCCTCCCAGCCCCCATCGAGAAAACCATCTCCAAAGCCAAAGGGCAGCCCCGAG
AACCACAGGTGTACACCCTGCCCCCATCCCGGGAGGAGATGACCAAGAACCAGGTCAGCCTGACC
TGCCTGGTCAAAGGCTTCTATCCCAGCGACATCGCCGTGGAGTGGGAGAGCAATGGGCAGCCGGA
GAACAACTACAAGACCACGCCTCCCGTGCTGGACTCCGACGGCTCCTTCTTCCTCTACAGCAAGCT
CACCGTGGACAAGAGCAGGTGGCAGCAGGGGAACGTCTTCTCATGCTCCGTGATGCATGAGGCTC
TGCACAACCACTACACGCAGAAGAGCCTCTCCCTGTCTCCGGGTAAA
LIGHT CHAIN
SEQ ID NO: 375

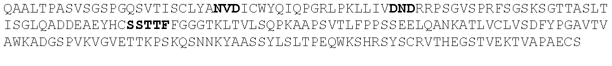

SEQ ID NO: 376
CAGGCTGCCCTGACTCAGCCCGCCTCCGTGTCCGGCTCTCCTGGACAGTCGGTCACCATTTCCTGC
CTTTATGCCAATGTAGATATCTGCTGGTATCAAATACAGCCGGGCAGATTACCCAAACTTCTGATT
GTTGACAATGATAGGCGACCCTCAGGAGTCTCTCCTCGCTTCTCTGGCTCCAAGTCTGGCACCACG
GCCTCCCTGACAATCTCTGGACTTCAGGCTGACGACGAGGCTGAATATCACTGCTCTTCAACAACA
TTTTTTGGCGGGGGGACCAAGTTGACCGTCCTCAGTCAGCCCAAGGCTGCCCCCTCGGTCACTCTG
TTCCCACCCTCCTCTGAGGAGCTTCAAGCCAACAAGGCCACACTGGTGTGTCTCGTAAGTGACTTC
TACCCGGGAGCCGTGACAGTGGCCTGGAAGGCAGATGGCAGCCCCGTCAAGGTGGGAGTGGAGA
CCACCAAACCCTCCAAACAAAGCAACAACAAGTATGCGGCCAGCAGCTACCTGAGCCTGACGCCC
GAGCAGTGGAAGTCCCACAGAAGCTACAGCTGCCGGGTCACGCATGAAGGGAGCACCGTGGAGA
AGACAGTGGCCCCTGCAGAATGCTCT
>N49P67
HEAVY CHAIN
SEQ ID NO: 377

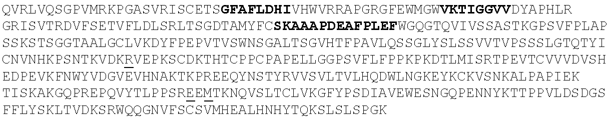

SEQ ID NO: 378
CAGGTGCGACTTGTGCAGTCTGGTCCCGTGATGAGAAAGCCTGGGGCCTCAGTGAGAATTTCTTGC
GAGACATCTGGATTCGCCTTCTTGGACCACATTGTCCACTGGGTGCGGCGGGCCCCTGGACGCGGC
TTTGAATGGATGGGTTGGGTTAAGACCATTGGGGGTGTCGTTGATTATGCACCCCACCTTAGGGGC
AGGATCTCCGTGACGAGAGACGTCTTTAGTGAAACCGTCTTTCTGGACTTGAGTCGACTGACGTCT
GGCGACACGGCGATGTATTTTTGTTCTAAGGCCGCCGCCCCTGACGAAGCCTTCCCCCTTGAATTT
TGGGGCCAGGGGACCCAAGTCATCGTCTCCTCGGCCTCCACCAAGGGCCCATCGGTCTTCCCCCTG
GCACCCTCCTCCAAGAGCACCTCTGGGGGCACAGCGGCCCTGGGCTGCCTGGTCAAGGACTACTT
CCCCGAACCGGTGACGGTGTCGTGGAACTCAGGCGCCCTGACCAGCGGCGTGCACACCTTCCCGG
CTGTCCTACAGTCCTCAGGACTCTACTCCCTCAGCAGCGTGGTGACCGTGCCCTCCAGCAGCTTGG
GCACCCAGACCTACATCTGCAACGTGAATCACAAGCCCAGCAACACCAAGGTGGACAAGAGAGTT
GAGCCCAAATCTTGTGACAAAACTCACACATGCCCACCGTGCCCAGCACCTGAACTCCTGGGGGG
ACCGTCAGTCTTCCTCTTCCCCCCAAAACCCAAGGACACCCTCATGATCTCCCGGACCCCTGAGGT
CACATGCGTGGTGGTGGACGTGAGCCACGAAGACCCTGAGGTCAAGTTCAACTGGTACGTGGACG
GCGTGGAGGTGCATAATGCCAAGACAAAGCCGCGGGAGGAGCAGTACAACAGCACGTACCGGGT
GGTCAGCGTCCTCACCGTCCTGCACCAGGACTGGCTGAATGGCAAGGAGTACAAGTGCAAGGTCT
CCAACAAAGCCCTCCCAGCCCCCATCGAGAAAACCATCTCCAAAGCCAAAGGGCAGCCCCGAGA
ACCACAGGTGTACACCCTGCCCCCATCCCGGGAGGAGATGACCAAGAACCAGGTCAGCCTGACCT
GCCTGGTCAAAGGCTTCTATCCCAGCGACATCGCCGTGGAGTGGGAGAGCAATGGGCAGCCGGAG
AACAACTACAAGACCACGCCTCCCGTGCTGGACTCCGACGGCTCCTTCTTCCTCTACAGCAAGCTC
ACCGTGGACAAGAGCAGGTGGCAGCAGGGGAACGTCTTCTCATGCTCCGTGATGCATGAGGCTCT
GCACAACCACTACACGCAGAAGAGCCTCTCCCTGTCTCCGGGTAAA
LIGHT CHAIN
SEQ ID NO: 379

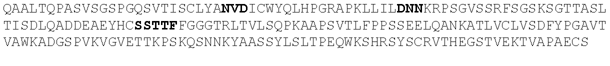

SEQ ID NO: 380
CAGGCTGCCCTGACTCAGCCCGCCTCCGTGTCCGGCTCTCCTGGACAGTCGGTCACCATTTCTTGC
CTTTATGCCAATGTGGATATCTGCTGGTATCAACTTCACCCGGGCAGAGCCCCCAAACTTCTAATT
CTTGACAATAATAAACGGCCCTCAGGAGTCTCTAGTCGCTTCTCCGGTTCCAAGTCTGGCACCACG
GCCTCCCTAACCATCTCTGACCTTCAGGCTGACGACGAGGCTGAATATCACTGCTCTTCAACAACT
TTTTTTGGCGGGGGGACCAGGTTGACCGTCCTGAGTCAGCCCAAGGCTGCCCCCTCGGTCACTCTG
TTCCCACCCTCCTCTGAGGAGCTTCAAGCCAACAAGGCCACACTGGTGTGTCTCGTAAGTGACTTC
TACCCGGGAGCCGTGACAGTGGCCTGGAAGGCAGATGGCAGCCCCGTCAAGGTGGGAGTGGAGA
CCACCAAACCCTCCAAACAAAGCAACAACAAGTATGCGGCCAGCAGCTACCTGAGCCTGACGCCC
GAGCAGTGGAAGTCCCACAGAAGCTACAGCTGCCGGGTCACGCATGAAGGGAGCACCGTGGAGA
AGACAGTGGCCCCTGCAGAATGCTCT
>N49P68
HEAVY CHAIN
SEQ ID NO: 381

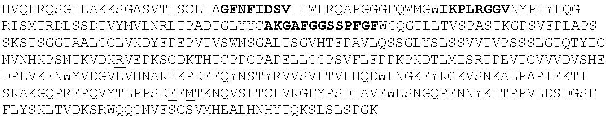

SEQ ID NO: 382
CACGTGCAGTTGAGGCAGTCTGGGACTGAGGCGAAGAAGTCTGGGGCCTCGGTGACAATCTCTTG
TGAGACCGCTGGATTCAACTTCATCGACTCCGTCATACACTGGCTGCGCCAGGCCCCTGGTGGGGG
ATTTCAGTGGATGGGGTGGATCAAGCCTCTTAGAGGTGGCGTCAATTATCCACATTATTITTGCAGGG
CAGAATCTCCATGACCCGGGACTTGTCCAGTGACACGGTTTACATGGTCTTAAATAGACTGACACC
TGCCGACACAGGCCTTTATTACTGCGCGAAAGGGGCCTTTGGGGGGAGTTCTCCCTTTGGCTTCTG
GGGCCAGGGAACTCTGCTCACCGTCTCCCCAGCCTCCACCAAGGGCCCATCGGTCTTCCCCCTGGC
ACCCTCCTCCAAGAGCACCTCTGGGGGCACAGCGGCCCTGGGCTGCCTGGTCAAGGACTACTTCC
CCGAACCGGTGACGGTGTCGTGGAACTCAGGCGCCCTGACCAGCGGCGTGCACACCTTCCCGGCT
GTCCTACAGTCCTCAGGACTCTACTCCCTCAGCAGCGTGGTGACCGTGCCCTCCAGCAGCTTGGGC
ACCCAGACCTACATCTGCAACGTGAATCACAAGCCCAGCAACACCAAGGTGGACAAGAGAGTTG
AGCCCAAATCTTGTGACAAAACTCACACATGCCCACCGTGCCCAGCACCTGAACTCCTGGGGGGA
CCGTCAGTCTTCCTCTTCCCCCCAAAACCCAAGGACACCCTCATGATCTCCCGGACCCCTGAGGTC
ACATGCGTGGTGGTGGACGTGAGCCACGAAGACCCTGAGGTCAAGTTCAACTGGTACGTGGACGG
CGTGGAGGTGCATAATGCCAAGACAAAGCCGCGGGAGGAGCAGTACAACAGCACGTACCGGGTG
GTCAGCGTCCTCACCGTCCTGCACCAGGACTGGCTGAATGGCAAGGAGTACAAGTGCAAGGTCTC
CAACAAAGCCCTCCCAGCCCCCATCGAGAAAACCATCTCCAAAGCCAAAGGGCAGCCCCGAGAA
CCACAGGTGTACACCCTGCCCCCATCCCGGGAGGAGATGACCAAGAACCAGGTCAGCCTGACCTG
CCTGGTCAAAGGCTTCTATCCCAGCGACATCGCCGTGGAGTGGGAGAGCAATGGGCAGCCGGAGA
ACAACTACAAGACCACGCCTCCCGTGCTGGACTCCGACGGCTCCTTCTTCCTCTACAGCAAGCTCA
CCGTGGACAAGAGCAGGTGGCAGCAGGGGAACGTCTTCTCATGCTCCGTGATGCATGAGGCTCTG
CACAACCACTACACGCAGAAGAGCCTCTCCCTGTCTCCGGGTAAA
LIGHT CHAIN
SEQ ID NO: 383

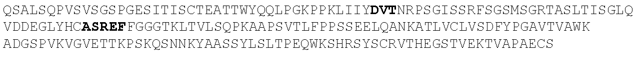

SEQ ID NO: 384
CAGTCTGCCCTGTCTCAGCCTGTCTCCGTGTCTGGGTCTCCTGGAGAGTCGATCACCATTTCCTGTA
CTGAAGCCACCACCTGGTATCAACAACTCCCAGGCAAACCCCCCAAACTCATCATTTATGACGTG
ACCAACCGGCCCTCAGGCATTTCAAGTCGTTTCTCTGGCTCCATGTCTGGTCGCACGGCCTCCCTG
ACAATCTCCGGTCTCCAGGTTGACGACGAGGGTCTCTATCACTGTGCCTCACGTGAATTTTTCGGC
GGGGGGACCAAGCTGACCGTCCTGAGTCAGCCCAAGGCTGCCCCCTCGGTCACTCTGTTCCCACCC
TCCTCTGAGGAGCTTCAAGCCAACAAGGCCACACTGGTGTGTCTCGTAAGTGACTTCTACCCGGGA
GCCGTGACAGTGGCCTGGAAGGCAGATGGCAGCCCCGTCAAGGTGGGAGTGGAGACCACCAAAC
CCTCCAAACAAAGCAACAACAAGTATGCGGCCAGCAGCTACCTGAGCCTGACGCCCGAGCAGTGG
AAGTCCCACAGAAGCTACAGCTGCCGGGTCACGCATGAAGGGAGCACCGTGGAGAAGACAGTGG
CCCCTGCAGAATGCTCT
>N49P69
HEAVY CHAIN
SEQ ID NO: 385

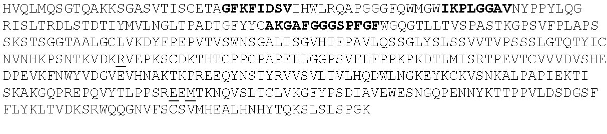

SEQ ID NO: 386
CACGTGCAATTGATGCAGTCTGGGACTCAGGCGAAGAAGTCTGGGGCCTCGGTGACAATTTCTTGT
GAGACCGCTGGATTCAAGTTCATCGACTCCGTCATACACTGGCTGCGCCAGGCCCCTGGAGGGGG
ATTTCAGTGGATGGGGTGGATCAAGCCTCTTGGAGGTGCCGTCAACTATCCACCCTATTTGCAGGG
CAGGATCTCCTTGACCCGTGACTTGTCCACCGACACAATTTACATGGTCTTGAATGGACTGACACC
TGCCGACACAGGCTTTTATTACTGCGCCAAAGGGGCCTTTGGGGGGGGTTCTCCCTTTGGCTTCTG
GGGCCAGGGGACTCTGCTCACCGTCTCCCCAGCCTCCACCAGGGCCCATCGGTCTTCCCCCTGGC
ACCCTCCTCCAAGAGCACCTCTGGGGGCACAGCGGCCCTGGGCTGCCTGGTCAAGGACTACTTCC
CCGAACCGGTGACGGTGTCGTGGAACTCAGGCGCCCTGACCAGCGGCGTGCACACCTTCCCGGCT
GTCCTACAGTCCTCAGGACTCTACTCCCTCAGCAGCGTGGTGACCGTGCCCTCCAGCAGCTTGGGC
ACCCAGACCTACATCTGCAACGTGAATCACAAGCCCAGCAACACCAAGGTGGACAAGAGAGTTG
AGCCCAAATCTTGTGACAAAACTCACACATGCCCACCGTGCCCAGCACCTGAACTCCTGGGGGGA
CCGTCAGTCTTCCTCTTCCCCCCAAAACCCAAGGACACCCTCATGATCTCCCGGACCCCTGAGGTC
ACATGCGTGGTGGTGGACGTGAGCCACGAAGACCCTGAGGTCAAGTTCAACTGGTACGTGGACGG
CGTGGAGGTGCATAATGCCAAGACAAAGCCGCGGGAGGAGCAGTACAACAGCACGTACCGGGTG
GTCAGCGTCCTCACCGTCCTGCACCAGGACTGGCTGAATGGCAAGGAGTACAAGTGCAAGGTCTC
CAACAAAGCCCTCCCAGCCCCCATCGAGAAAACCATCTCCAAAGCCAAAGGGCAGCCCCGAGAA
CCACAGGTGTACACCCTGCCCCCATCCCGGGAGGAGATGACCAAGAACCAGGTCAGCCTGACCTG
CCTGGTCAAAGGCTTCTATCCCAGCGACATCGCCGTGGAGTGGGAGAGCAATGGGCAGCCGGAGA
ACAACTACAAGACCACGCCTCCCGTGCTGGACTCCGACGGCTCCTTCTTCCTCTACAGCAAGCTCA
CCGTGGACAAGAGCAGGTGGCAGCAGGGGAACGTCTTCTCATGCTCCGTGATGCATGAGGCTCTG
CACAACCACTACACGCAGAAGAGCCTCTCCCTGTCTCCGGGTAAA
LIGHT CHAIN
SEQ ID NO: 387

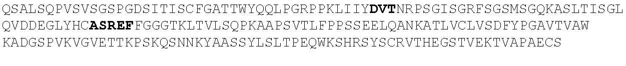

SEQ ID NO: 388
CAGTCTGCCCTGTCTCAGCCTGTCTCCGTGTCTGGGTCTCCTGGAGACTCGATCACCATTTCTTGTT
TTGGAGCCACCACCTGGTATCAACAACTCCCAGGCAGACCCCCCAAACTCATCATTTATGACGTGA
CTAACCGGCCCTCAGGCATTTCAGGTCGTTTCTCTGGCTCCATGTCTGGTCAAAAGGCCTCCCTGA
CAATCTCCGGTCTCCAGGTTGACGACGAGGGTCTCTATCACTGCGCCTCACGTGAATTTTTCGGCG
GGGGGACCAAACTGACCGTCCTGAGTCAGCCCAAGGCTGCCCCCTCGGTCACTCTGTTCCCACCCT
CCTCTGAGGAGCTTCAAGCCAACAAGGCCACACTGGTGTGTCTCGTAAGTGACTTCTACCCGGGA
GCCGTGACAGTGGCCTGGAAGGCAGATGGCAGCCCCGTCAAGGTGGGAGTGGAGACCACCAAAC
CCTCCAAACAAAGCAACAACAAGTATGCGGCCAGCAGCTACCTGAGCCTGACGCCCGAGCAGTGG
AAGTCCCACAGAAGCTACAGCTGCCGGGTCACGCATGAAGGGAGCACCGTGGAGAAGACAGTGG
CCCCTGCAGAATGCTCT
>N49P70
HEAVY CHAIN
SEQ ID NO: 389

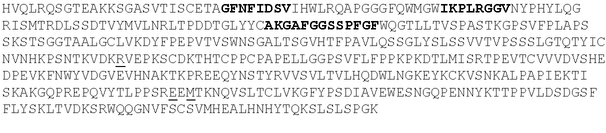

SEQ ID NO: 390
CACGTGCAGTTGAGGCAGTCTGGGACTGAGGCGAAGAAGTCTGGGGCCTCGGTGACAATCTCTTG
TGAGACCGCTGGATTCAACTTCATCGACTCCGTCATACACTGGCTGCGCCAGGCCCCTGGTGGGGG
ATTTCAGTGGATGGGGTGGATCAAGCCTCTTAGAGGTGGCGTCAATTATCCACATTATTITTGCAGGG
CAGAATCTCCATGACCCGGGACTTGTCCAGTGACACGGTTTACATGGTCTTAAATAGACTGACACC
TGACGACACAGGCCTTTACTACTGCGCGAAAGGGGCCTTTGGGGGGAGTTCTCCCTTTGGCTTCTG
GGGCCAGGGAACTCTGCTCACCGTCTCCCCAGCCTCCACCAAGGGCCCATCGGTCTTCCCCCTGGC
ACCCTCCTCCAAGAGCACCTCTGGGGGCACAGCGGCCCTGGGCTGCCTGGTCAAGGACTACTTCC
CCGAACCGGTGACGGTGTCGTGGAACTCAGGCGCCCTGACCAGCGGCGTGCACACCTTCCCGGCT
GTCCTACAGTCCTCAGGACTCTACTCCCTCAGCAGCGTGGTGACCGTGCCCTCCAGCAGCTTGGGC
ACCCAGACCTACATCTGCAACGTGAATCACAAGCCCAGCAACACCAAGGTGGACAAGAGAGTTG
AGCCCAAATCTTGTGACAAAACTCACACATGCCCACCGTGCCCAGCACCTGAACTCCTGGGGGGA
CCGTCAGTCTTCCTCTTCCCCCCAAAACCCAAGGACACCCTCATGATCTCCCGGACCCCTGAGGTC
ACATGCGTGGTGGTGGACGTGAGCCACGAAGACCCTGAGGTCAAGTTCAACTGGTACGTGGACGG
CGTGGAGGTGCATAATGCCAAGACAAAGCCGCGGGAGGAGCAGTACAACAGCACGTACCGGGTG
GTCAGCGTCCTCACCGTCCTGCACCAGGACTGGCTGAATGGCAAGGAGTACAAGTGCAAGGTCTC
CAACAAAGCCCTCCCAGCCCCCATCGAGAAAACCATCTCCAAAGCCAAAGGGCAGCCCCGAGAA
CCACAGGTGTACACCCTGCCCCCATCCCGGGAGGAGATGACCAAGAACCAGGTCAGCCTGACCTG
CCTGGTCAAAGGCTTCTATCCCAGCGACATCGCCGTGGAGTGGGAGAGCAATGGGCAGCCGGAGA
ACAACTACAAGACCACGCCTCCCGTGCTGGACTCCGACGGCTCCTTCTTCCTCTACAGCAAGCTCA
CCGTGGACAAGAGCAGGTGGCAGCAGGGGAACGTCTTCTCATGCTCCGTGATGCATGAGGCTCTG
CACAACCACTACACGCAGAAGAGCCTCTCCCTGTCTCCGGGTAAA
LIGHT CHAIN
SEQ ID NO: 391

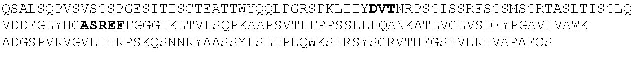

SEQ ID NO: 392
CAGTCTGCCCTGTCTCAGCCTGTCTCCGTGTCTGGGTCTCCTGGAGAGTCGATCACCATTTCCTGTA
CTGAAGCCACCACCTGGTATCAACAACTCCCAGGGAGATCCCCCAAACTCATTATTTATGACGTGA
CCAACCGGCCCTCAGGCATTTCAAGTCGTTTCTCTGGCTCCATGTCTGGTCGCACGGCCTCCCTGA
CAATCTCCGGTCTCCAGGTTGACGACGAGGGTCTCTATCACTGTGCCTCACGTGAATTTTTCGGCG
GGGGGACCAAGCTGACCGTCCTCAGTCAGCCCAAGGCTGCCCCCTCGGTCACTCTGTTCCCACCCT
CCTCTGAGGAGCTTCAAGCCAACAAGGCCACACTGGTGTGTCTCGTAAGTGACTTCTACCCGGGA
GCCGTGACAGTGGCCTGGAAGGCAGATGGCAGCCCCGTCAAGGTGGGAGTGGAGACCACCAAAC
CCTCCAAACAAAGCAACAACAAGTATGCGGCCAGCAGCTACCTGAGCCTGACGCCCGAGCAGTGG
AAGTCCCACAGAAGCTACAGCTGCCGGGTCACGCATGAAGGGAGCACCGTGGAGAAGACAGTGG
CCCCTGCAGAATGCTCT
>N49P71
HEAVY CHAIN
SEQ ID NO: 393

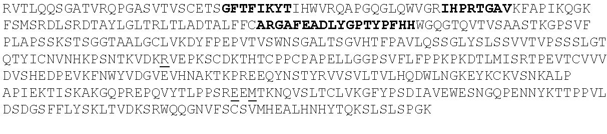

SEQ ID NO: 394
CGTGTTACATTACAACAGTCTGGGGCTACAGTGAGGCAGCCTGGGGCCTCAGTCACCGTCTCCTGC
GAGACTTCTGGATTCACCTTCATCAAATATACCATTCACTGGGTGCGACAGGCCCCTGGACAGGGT
CTTCAGTGGGTGGGAAGAATACACCCCCGAACCGGTGCCGTGAAGTTTGCACCGATATTTCAGGG
TAAATTTTCCATGAGTCGAGACTTGTCACGCGACACAGCCTACCTCGGATTGACCAGACTGACACT
CGCCGACACGGCTCTATTTTTCTGTGCGAGGGGGGCCTTTGAGGCAGATTTATATGGGCCAACTTA
CCCCTTTCACCACTGGGGCCAAGGAACCCAAGTCACCGTCTCCGCGGCCTCCACCAAGGGCCCAT
CGGTCTTCCCCCTGGCACCCTCCTCCAAGAGCACCTCTGGGGGCACAGCGGCCCTGGGCTGCCTGG
TCAAGGACTACTTCCCCGAACCGGTGACGGTGTCGTGGAACTCAGGCGCCCTGACCAGCGGCGTG
CACACCTTCCCGGCTGTCCTACAGTCCTCAGGACTCTACTCCCTCAGCAGCGTGGTGACCGTGCCC
TCCAGCAGCTTGGGCACCCAGACCTACATCTGCAACGTGAATCACAAGCCCAGCAACACCAAGGT
GGACAAGAGAGTTGAGCCCAAATCTTGTGACAAAACTCACACATGCCCACCGTGCCCAGCACCTG
AACTCCTGGGGGGACCGTCAGTCTTCCTCTTCCCCCCAAAACCCAAGGACACCCTCATGATCTCCC
GGACCCCTGAGGTCACATGCGTGGTGGTGGACGTGAGCCACGAAGACCCTGAGGTCAAGTTCAAC
TGGTACGTGGACGGCGTGGAGGTGCATAATGCCAAGACAAAGCCGCGGGAGGAGCAGTACAACA
GCACGTACCGGGTGGTCAGCGTCCTCACCGTCCTGCACCAGGACTGGCTGAATGGCAAGGAGTAC
AAGTGCAAGGTCTCCAACAAAGCCCTCCCAGCCCCCATCGAGAAAACCATCTCCAAAGCCAAAGG
GCAGCCCCGAGAACCACAGGTGTACACCCTGCCCCCATCCCGGGAGGAGATGACCAAGAACCAG
GTCAGCCTGACCTGCCTGGTCAAAGGCTTCTATCCCAGCGACATCGCCGTGGAGTGGGAGAGCAA
TGGGCAGCCGGAGAACAACTACAAGACCACGCCTCCCGTGCTGGACTCCGACGGCTCCTTCTTCCT
CTACAGCAAGCTCACCGTGGACAAGAGCAGGTGGCAGCAGGGGAACGTCTTCTCATGCTCCGTGA
TGCATGAGGCTCTGCACAACCACTACACGCAGAAGAGCCTCTCCCTGTCTCCGGGTAAA
LIGHT CHAIN
SEQ ID NO: 395

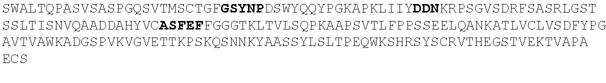

SEQ ID NO: 396
TCCTGGGCCCTGACTCAACCCGCCTCCGTGTCTGCGTCTCCTGGGCAGTCGGTCACCATGTCCTGC
ACTGGATTCGGAAGTTATAATCCTGACTCCTGGTACCAGCAATACCCAGGCAAAGCCCCCAAACT
CATCATTTATGATGACAATAAAAGACCCTCGGGGGTCTCTGATCGCTTCTCTGCCTCCAGACTTGG
CAGCACATCTTCACTGACAATCTCTAACGTCCAGGCCGCTGACGACGCCCATTATGTCTGCGCCTC
CTTTGAGTTTTTCGGCGGGGGGACCAAGCTGACCGTCCTGAGTCAGCCCAAGGCTGCCCCCTCGGT
CACTCTGTTCCCACCCTCCTCTGAGGAGCTTCAAGCCAACAAGGCCACACTGGTGTGTCTCGTAAG
TGACTTCTACCCGGGAGCCGTGACAGTGGCCTGGAAGGCAGATGGCAGCCCCGTCAAGGTGGGAG
TGGAGACCACCAAACCCTCCAAACAAAGCAACAACAAGTATGCGGCCAGCAGCTACCTGAGCCTG
ACGCCCGAGCAGTGGAAGTCCCACAGAAGCTACAGCTGCCGGGTCACGCATGAAGGGAGCACCG
TGGAGAAGACAGTGGCCCCTGCAGAATGCTCT
>N49P72
HEAVY CHAIN
SEQ ID NO: 397

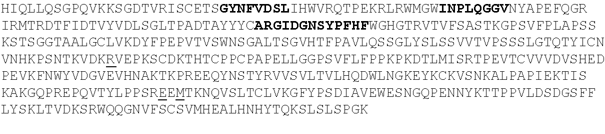

SEQ ID NO: 398
CACATTCAGTTGCTACAGTCGGGGCCTCAAGTTAAGAAGTCTGGGGACACAGTGAGAATCTCCTG
TGAGACCTCTGGATATAATTTCGTCGACTCCCTTATCCACTGGGTCCGACAGACCCCGGAAAAACG
TCTCAGATGGATGGGCTGGATCAATCCTCTCCAAGGTGGTGTGAATTACGCGCCGGAATTTCAGGG
CAGAATCAGGATGACCAGGGACACGTTTATAGACACAGTTTACGTGGACTTGAGCGGACTGACAC
CGGCCGACACGGCCTATTATTACTGCGCGCGAGGGATCGATGGCAATTCTTACCCCTTTCATTTCT
GGGGCCACGGAACCCGGGTCACCGTCTTCTCGGCCTCCACCAAGGGCCCATCGGTCTTCCCCCTGG
CACCCTCCTCCAAGAGCACCTCTGGGGGCACAGCGGCCCTGGGCTGCCTGGTCAAGGACTACTTC
CCCGAACCGGTGACGGTGTCGTGGAACTCAGGCGCCCTGACCAGCGGCGTGCACACCTTCCCGGC
TGTCCTACAGTCCTCAGGACTCTACTCCCTCAGCAGCGTGGTGACCGTGCCCTCCAGCAGCTTGGG
CACCCAGACCTACATCTGCAACGTGAATCACAAGCCCAGCAACACCAAGGTGGACAAGAGAGTTG
AGCCCAAATCTTGTGACAAAACTCACACATGCCCACCGTGCCCAGCACCTGAACTCCTGGGGGGA
CCGTCAGTCTTCCTCTTCCCCCCAAAACCCAAGGACACCCTCATGATCTCCCGGACCCCTGAGGTC
ACATGCGTGGTGGTGGACGTGAGCCACGAAGACCCTGAGGTCAAGTTCAACTGGTACGTGGACGG
CGTGGAGGTGCATAATGCCAAGACAAAGCCGCGGGAGGAGCAGTACAACAGCACGTACCGGGTG
GTCAGCGTCCTCACCGTCCTGCACCAGGACTGGCTGAATGGCAAGGAGTACAAGTGCAAGGTCTC
CAACAAAGCCCTCCCAGCCCCCATCGAGAAAACCATCTCCAAAGCCAAAGGGCAGCCCCGAGAA
CCACAGGTGTACACCCTGCCCCCATCCCGGGAGGAGATGACCAAGAACCAGGTCAGCCTGACCTG
CCTGGTCAAAGGCTTCTATCCCAGCGACATCGCCGTGGAGTGGGAGAGCAATGGGCAGCCGGAGA
ACAACTACAAGACCACGCCTCCCGTGCTGGACTCCGACGGCTCCTTCTTCCTCTACAGCAAGCTCA
CCGTGGACAAGAGCAGGTGGCAGCAGGGGAACGTCTTCTCATGCTCCGTGATGCATGAGGCTCTG
CACAACCACTACACGCAGAAGAGCCTCTCCCTGTCTCCGGGTAAA
LIGHT CHAIN
SEQ ID NO: 399

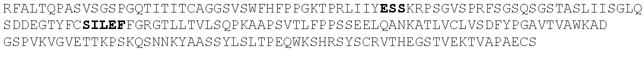

SEQ ID NO: 400
CGATTTGCCCTGACTCAACCTGCCTCCGTGTCTGGGTCTCCTGGACAGACGATCACCATAACCTGC
GCTGGAGGCAGCGTCTCCTGGTTCCATTTCCCTCCAGGCAAAACCCCCAGACTCATTATTTATGAG
TCTTCTAAGAGACCCTCAGGGGTCTCTCCTCGATTCTCTGGGTCCCAGTCTGGCAGCACGGCCTCC
CTAATAATTTCTGGCCTCCAGTCTGATGACGAAGGGACATACTTCTGTTCTATTCTTGAATTTTTCG
GCAGAGGGACTCTTCTCACCGTCCTGAGTCAGCCCAAGGCTGCCCCCTCGGTCACTCTGTTCCCAC
CCTCCTCTGAGGAGCTTCAAGCCAACAAGGCCACACTGGTGTGTCTCGTAAGTGACTTCTACCCGG
GAGCCGTGACAGTGGCCTGGAAGGCAGATGGCAGCCCCGTCAAGGTGGGAGTGGAGACCACCAA
ACCCTCCAAACAAAGCAACAACAAGTATGCGGCCAGCAGCTACCTGAGCCTGACGCCCGAGCAGT
GGAAGTCCCACAGAAGCTACAGCTGCCGGGTCACGCATGAAGGGAGCACCGTGGAGAAGACAGT
GGCCCCTGCAGAATGCTCT
>N49P7-F4
HEAVY CHAIN
SEQ ID NO: 501

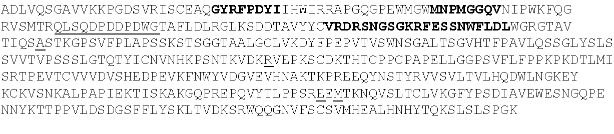

SEQ ID NO: 502
GCGGACTTGGTGCAGTCTGGGGCTGTGGTGAAGAAGCCTGGGGACTCAGTGAGGATCTCCTGTGA
GGCTCAAGGATATAGATTTCCTGACTACATCATTCACTGGATTCGACGGGCCCCTGGACAAGGCCC
TGAATGGATGGGATGGATGAATCCAATGGGCGGACAAGTAAATATTCCATGGAAATTTCAGGGTA
GGGTCTCCATGACCCGGCAATTATCTCAAGACCCAGACGACCCGGACTGGGGCACAGCATTTCTG
GACTTAAGAGGACTAAAGTCTGACGACACGGCCGTCTATTATTGCGTGAGAGATCGCAGTAATGG
ATCGGGAAAGCGATTCGAGTCCTCCAATTGGTTCCTCGATCTGTGGGGCCGTGGGACTGCGGTCAC
AATTCAATCAGCCTCCACCAAGGGCCCATCGGTCTTCCCCCTGGCACCCTCCTCCAAGAGCACCTC
TGGGGGCACAGCGGCCCTGGGCTGCCTGGTCAAGGACTACTTCCCCGAACCGGTGACGGTGTCGT
GGAACTCAGGCGCCCTGACCAGCGGCGTGCACACCTTCCCGGCTGTCCTACAGTCCTCAGGACTCT
ACTCCCTCAGCAGCGTGGTGACCGTGCCCTCCAGCAGCTTGGGCACCCAGACCTACATCTGCAAC
GTGAATCACAAGCCCAGCAACACCAAGGTGGACAAGAGAGTTGAGCCCAAATCTTGTGACAAAA
CTCACACATGCCCACCGTGCCCAGCACCTGAACTCCTGGGGGGACCGTCAGTCTTCCTCTTCCCCC
CAAAACCCAAGGACACCCTCATGATCTCCCGGACCCCTGAGGTCACATGCGTGGTGGTGGACGTG
AGCCACGAAGACCCTGAGGTCAAGTTCAACTGGTACGTGGACGGCGTGGAGGTGCATAATGCCAA
GACAAAGCCGCGGGAGGAGCAGTACAACAGCACGTACCGGGTGGTCAGCGTCCTCACCGTCCTGC
ACCAGGACTGGCTGAATGGCAAGGAGTACAAGTGCAAGGTCTCCAACAAAGCCCTCCCAGCCCCC
ATCGAGAAAACCATCTCCAAAGCCAAAGGGCAGCCCCGAGAACCACAGGTGTACACCCTGCCCCC
ATCCCGGGAGGAGATGACCAAGAACCAGGTCAGCCTGACCTGCCTGGTCAAAGGCTTCTATCCCA
GCGACATCGCCGTGGAGTGGGAGAGCAATGGGCAGCCGGAGAACAACTACAAGACCACGCCTCC
CGTGCTGGACTCCGACGGCTCCTTCTTCCTCTACAGCAAGCTCACCGTGGACAAGAGCAGGTGGC
AGCAGGGGAACGTCTTCTCATGCTCCGTGATGCATGAGGCTCTGCACAACCACTACACGCAGAAG
AGCCTCTCCCTGTCTCCGGGTAAA
LIGHT CHAIN
SEQ ID NO: 503

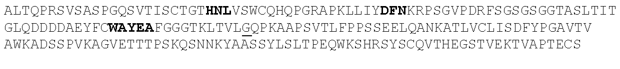

SEQ ID NO: 504
GCCCTGACTCAGCCTCGCTCAGTGTCCGCATCTCCTGGGCAGTCCGTCACCATTTCCTGCACTGGA
ACCCACAATTTGGTCTCTTGGTGTCAACATCAGCCAGGCAGAGCCCCCAAATTATTAATTTATGAC
TTCAATAAGAGGCCCTCAGGGGTCCCTGATCGCTTCTCTGGCTCCGGGTCTGGCGGCACGGCCTCC
CTGACCATCACTGGACTCCAGGATGACGATGACGCCGAATATTTTTGTTGGGCGTATGAAGCTTTT
GGCGGAGGGACCAAGTTGACCGTTCTTGGTCAGCCCAAGGCTGCCCCCTCGGTCACTCTGTTCCCG
CCCTCCTCTGAGGAGCTTCAAGCCAACAAGGCCACACTGGTGTGTCTCATAAGTGACTTCTACCCG
GGAGCCGTGACAGTGGCTTGGAAAGCAGATAGCAGCCCCGTCAAGGCGGGAGTGGAGACCACCA
CACCCTCCAAACAAAGCAACAACAAGTACGCGGCCAGCAGCTATCTGAGCCTGACGCCTGAGCAG
TGGAAGTCCCACAGAAGCTACAGCTGCCAGGTCACGCATGAAGGGAGCACCGTGGAGAAGACAG
TGGCCCCTACAGAATGTTCA
>N49P9-FR
HEAVY CHAIN
SEQ ID NO: 505

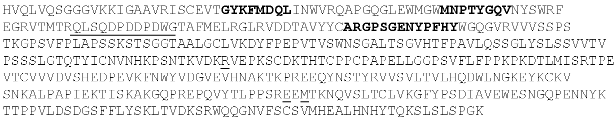

SEQ ID NO: 506
CACGTCCAATTGGTGCAGTCTGGAGGTGGGGTGAAGAAGATTGGGGCCGCTGTGAGGATCTCCTG
CGAGGTGACTGGATATAAATTCATGGACCAACTCATAAACTGGGTGCGGCAGGCCCCCGGTCAGG
GCCTTGAGTGGATGGGATGGATGAATCCAACATATGGACAAGTAAATTATTCATGGAGATTTGAA
GGAAGGGTCACCATGACCAGGCAATTATCTCAAGACCCAGACGACCCGGACTGGGGCACGGCCTT
CATGGAGTTGAGAGGACTGAGAGTGGACGACACGGCCGTCTATTATTGCGCGAGGGGACCCTCTG
GGGAAAATTATCCTTTTCACTATTGGGGCCAGGGTGTCCGAGTGGTCGTCTCGTCACCCTCCACCA
AGGGCCCATCGGTCTTCCCCCTGGCACCCTCCTCCAAGAGCACCTCTGGGGGCACAGCGGCCCTG
GGCTGCCTGGTCAAGGACTACTTCCCCGAACCGGTGACGGTGTCGTGGAACTCAGGCGCCCTGAC
CAGCGGCGTGCACACCTTCCCGGCTGTCCTACAGTCCTCAGGACTCTACTCCCTCAGCAGCGTGGT
GACCGTGCCCTCCAGCAGCTTGGGCACCCAGACCTACATCTGCAACGTGAATCACAAGCCCAGCA
ACACCAAGGTGGACAAGAGAGTTGAGCCCAAATCTTGTGACAAAACTCACACATGCCCACCGTGC
CCAGCACCTGAACTCCTGGGGGGACCGTCAGTCTTCCTCTTCCCCCCAAAACCCAAGGACACCCTC
ATGATCTCCCGGACCCCTGAGGTCACATGCGTGGTGGTGGACGTGAGCCACGAAGACCCTGAGGT
CAAGTTCAACTGGTACGTGGACGGCGTGGAGGTGCATAATGCCAAGACAAAGCCGCGGGAGGAG
CAGTACAACAGCACGTACCGGGTGGTCAGCGTCCTCACCGTCCTGCACCAGGACTGGCTGAATGG
CAAGGAGTACAAGTGCAAGGTCTCCAACAAAGCCCTCCCAGCCCCCATCGAGAAAACCATCTCCA
AAGCCAAAGGGCAGCCCCGAGAACCACAGGTGTACACCCTGCCCCCATCCCGGGAGGAGATGAC
CAAGAACCAGGTCAGCCTGACCTGCCTGGTCAAAGGCTTCTATCCCAGCGACATCGCCGTGGAGT
GGGAGAGCAATGGGCAGCCGGAGAACAACTACAAGACCACGCCTCCCGTGCTGGACTCCGACGG
CTCCTTCTTCCTCTACAGCAAGCTCACCGTGGACAAGAGCAGGTGGCAGCAGGGGAACGTCTTCTC
ATGCTCCGTGATGCATGAGGCTCTGCACAACCACTACACGCAGAAGAGCCTCTCCCTGTCTCCGGG
TAAA
LIGHT CHAIN
SEQ ID NO: 295

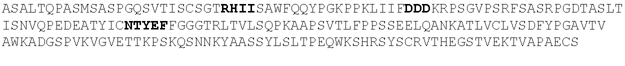

SEQ ID NO: 296
GCATCTGCCCTGACTCAGCCTGCCTCCATGTCTGCGTCCCCTGGACAGTCGGTAACCATCTCGTGC
TCTGGAACCAGACACATAATCTCTGCTTGGTTCCAACAATATCCAGGCAAACCACCCAAACTCATA
ATTTTTGACGACGATAAGCGTCCCTCTGGAGTTCCTAGTCGCTTCTCTGCCTCCAGGCCTGGCGAC
ACGGCCTCCCTGACAATCTCTAATGTTCAACCTGAGGACGAGGCGACGTACATTTGCAATACATAT
GAATTCTTTGGCGGAGGGACCAGATTGACCGTCCTAAGTCAGCCCAAGGCTGCCCCCTCGGTCACT
CTGTTCCCACCCTCCTCTGAGGAGCTTCAAGCCAACAAGGCCACACTGGTGTGTCTCGTAAGTGAC
TTCTACCCGGGAGCCGTGACAGTGGCCTGGAAGGCAGATGGCAGCCCCGTCAAGGTGGGAGTGGA
GACCACCAAACCCTCCAAACAAAGCAACAACAAGTATGCGGCCAGCAGCTACCTGAGCCTGACGC
CCGAGCAGTGGAAGTCCCACAGAAGCTACAGCTGCCGGGTCACGCATGAAGGGAGCACCGTGGA
GAAGACAGTGGCCCCTGCAGAATGCTCT
>N49P9.3-FR
HEAVY CHAIN
SEQ ID NO: 507

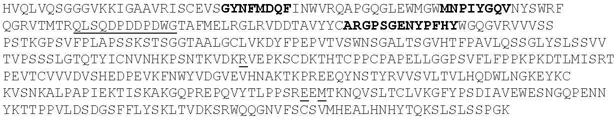

SEQ ID NO: 508
CACGTCCAATTGGTGCAGTCTGGAGGTGGGGTGAAGAAGATTGGGGCCGCTGTGAGGATCTCCTG
CGAGGTGTCTGGATACAACTTCATGGACCAATTCATAAATTGGGTGCGACAGGCCCCCGGTCAGG
GCCTTGAGTGGATGGGATGGATGAACCCAATATATGGACAAGTAAATTATTCATGGAGATTTCAA
GGAAGGGTCACCATGACCAGGCAATTATCTCAAGACCCAGACGACCCGGACTGGGGCACGG
CCTTCATGGAGTTGAGAGGACTGAGAGTGGACGACACGGCCGTCTATTATTGCGCGAGGGGACCC
TCTGGGGAAAATTATCCTTTTCACTATTGGGGCCAGGGTGTCCGAGTGGTCGTCTCGTCACCCTCC
ACCAAGGGCCCATCGGTCTTCCCCCTGGCACCCTCCTCCAAGAGCACCTCTGGGGGCACAGCGGC
CCTGGGCTGCCTGGTCAAGGACTACTTCCCCGAACCGGTGACGGTGTCGTGGAACTCAGGCGCCC
TGACCAGCGGCGTGCACACCTTCCCGGCTGTCCTACAGTCCTCAGGACTCTACTCCCTCAGCAGCG
TGGTGACCGTGCCCTCCAGCAGCTTGGGCACCCAGACCTACATCTGCAACGTGAATCACAAGCCC
AGCAACACCAAGGTGGACAAGAGAGTTGAGCCCAAATCTTGTGACAAAACTCACACATGCCCACC
GTGCCCAGCACCTGAACTCCTGGGGGGACCGTCAGTCTTCCTCTTCCCCCCAAAACCCAAGGACAC
CCTCATGATCTCCCGGACCCCTGAGGTCACATGCGTGGTGGTGGACGTGAGCCACGAAGACCCTG
AGGTCAAGTTCAACTGGTACGTGGACGGCGTGGAGGTGCATAATGCCAAGACAAAGCCGCGGGA
GGAGCAGTACAACAGCACGTACCGGGTGGTCAGCGTCCTCACCGTCCTGCACCAGGACTGGCTGA
ATGGCAAGGAGTACAAGTGCAAGGTCTCCAACAAAGCCCTCCCAGCCCCCATCGAGAAAACCATC
TCCAAAGCCAAAGGGCAGCCCCGAGAACCACAGGTGTACACCCTGCCCCCATCCCGGGAGGAGAT
GACCAAGAACCAGGTCAGCCTGACCTGCCTGGTCAAAGGCTTCTATCCCAGCGACATCGCCGTGG
AGTGGGAGAGCAATGGGCAGCCGGAGAACAACTACAAGACCACGCCTCCCGTGCTGGACTCCGA
CGGCTCCTTCTTCCTCTACAGCAAGCTCACCGTGGACAAGAGCAGGTGGCAGCAGGGGAACGTCT
TCTCATGCTCCGTGATGCATGAGGCTCTGCACAACCACTACACGCAGAAGAGCCTCTCCCTGTCTC
CGGGTAAA
LIGHT CHAIN
SEQ ID NO: 327

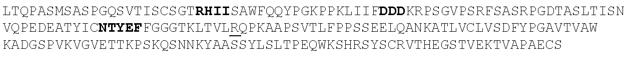

SEQ ID NO: 328
CTGACTCAGCCTGCCTCCATGTCTGCGTCCCCTGGACAGTCGGTAACCATCTCGTGCTCTGGAACC
AGACACATAATCTCTGCTTGGTTCCAACAATATCCAGGCAAACCACCCAAACTCATAATTTTTGAC
GACGATAAGCGTCCCTCTGGAGTTCCTAGTCGCTTCTCTGCCTCCAGGCCTGGCGACACGGCCTCC
CTGACAATCTCTAATGTTCAACCTGAGGACGAGGCGACATACATTTGCAATACATATGAATTCTTT
GGCGGAGGGACCAAATTGACCGTCCTACGTCAGCCCAAGGCTGCCCCCTCGGTCACTCTGTTCCCA
CCCTCCTCTGAGGAGCTTCAAGCCAACAAGGCCACACTGGTGTGTCTCGTAAGTGACTTCTACCCG
GGAGCCGTGACAGTGGCCTGGAAGGCAGATGGCAGCCCCGTCAAGGTGGGAGTGGAGACCACCA
AACCCTCCAAACAAAGCAACAACAAGTATGCGGCCAGCAGCTACCTGAGCCTGACGCCCGAGCA
GTGGAAGTCCCACAGAAGCTACAGCTGCCGGGTCACGCATGAAGGGAGCACCGTGGAGAAGACA
GTGGCCCCTGCAGAATGCTCT
>N49P9.6-FR
HEAVY CHAIN
SEQ ID NO: 509

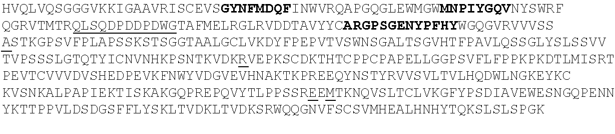

SEQ ID NO: 510
CACGTCCAATTGGTGCAGTCTGGAGGTGGGGTGAAGAAGATTGGGGCCGCTGTGAGGATCTCCTG
CGAGGTGTCTGGATACAACTTCATGGACCAATTCATAAATTGGGTGCGACAGGCCCCCGGTCAGG
GCCTTGAGTGGATGGGATGGATGAACCCAATATATGGACAAGTAAATTATTCATGGAGATTTCAA
GGAAGGGTCACCATGACCAGGCAATTATCTCAAGACCCAGACGACCCGGACTGGGGCACGG
CCTTCATGGAGTTGAGAGGACTGAGAGTGGACGACACGGCCGTCTATTATTGCGCGAGGGGACCC
TCTGGGGAAAATTATCCTTTTCACTATTGGGGCCAGGGTGTCCGAGTGGTCGTCTCGTCAGCGTCC
ACCAAGGGCCCATCGGTCTTCCCCCTGGCACCCTCCTCCAAGAGCACCTCTGGGGGCACAGCGGC
CCTGGGCTGCCTGGTCAAGGACTACTTCCCCGAACCGGTGACGGTGTCGTGGAACTCAGGCGCCC
TGACCAGCGGCGTGCACACCTTCCCGGCTGTCCTACAGTCCTCAGGACTCTACTCCCTCAGCAGCG
TGGTGACCGTGCCCTCCAGCAGCTTGGGCACCCAGACCTACATCTGCAACGTGAATCACAAGCCC
AGCAACACCAAGGTGGACAAGAGAGTTGAGCCCAAATCTTGTGACAAAACTCACACATGCCCACC
GTGCCCAGCACCTGAACTCCTGGGGGGACCGTCAGTCTTCCTCTTCCCCCCAAAACCCAAGGACAC
CCTCATGATCTCCCGGACCCCTGAGGTCACATGCGTGGTGGTGGACGTGAGCCACGAAGACCCTG
AGGTCAAGTTCAACTGGTACGTGGACGGCGTGGAGGTGCATAATGCCAAGACAAAGCCGCGGGA
GGAGCAGTACAACAGCACGTACCGGGTGGTCAGCGTCCTCACCGTCCTGCACCAGGACTGGCTGA
ATGGCAAGGAGTACAAGTGCAAGGTCTCCAACAAAGCCCTCCCAGCCCCCATCGAGAAAACCATC
TCCAAAGCCAAAGGGCAGCCCCGAGAACCACAGGTGTACACCCTGCCCCCATCCCGGGAGGAGAT
GACCAAGAACCAGGTCAGCCTGACCTGCCTGGTCAAAGGCTTCTATCCCAGCGACATCGCCGTGG
AGTGGGAGAGCAATGGGCAGCCGGAGAACAACTACAAGACCACGCCTCCCGTGCTGGACTCCGA
CGGCTCCTTCTTCCTCTACAGCAAGCTCACCGTGGACAAGAGCAGGTGGCAGCAGGGGAACGTCT
TCTCATGCTCCGTGATGCATGAGGCTCTGCACAACCACTACACGCAGAAGAGCCTCTCCCTGTCTC
CGGGTAAA
LIGHT CHAIN
SEQ ID NO: 511

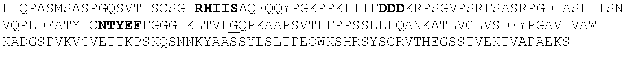

SEQ ID NO: 512
CTGACTCAGCCTGCCTCCATGTCTGCGTCCCCTGGACAGTCGGTAACCATCTCGTGCTCTGGAACC
AGACACATAATCTCTGCTTGGTTCCAACAATATCCAGGCAAACCACCCAAACTCATAATTTTTGAC
GACGATAAGCGTCCCTCTGGAGTTCCTAGTCGCTTCTCTGCCTCCAGGCCTGGCGACACGGCCTCC
CTGACAATCTCTAATGTTCAACCTGAGGACGAGGCGACATACATTTGCAATACATATGAATTCTTT
GGCGGAGGGACCAAATTGACCGTCCTAGGTCAGCCCAAGGCTGCCCCCTCGGTCACTCTGTITTCCC
ACCCTCCTCTGAGGAGCTTCAAGCCAACAAGGCCACACTGGTGTGTCTCGTAAGTGACTTCTACCC
GGGAGCCGTGACAGTGGCCTGGAAGGCAGATGGCAGCCCCGTCAAGGTGGGAGTGGAGACCACC
AAACCCTCCAAACAAAGCAACAACAAGTATGCGGCCAGCAGCTACCTGAGCCTGACGCCCGAGC
AGTGGAAGTCCCACAGAAGCTACAGCTGCCGGGTCACGCATGAAGGGAGCACCGTGGAGAAGAC
AGTGGCCCCTGCAGAATGCTCT
>N49P9.6-FR-54W
HEAVY CHAIN
SEQ ID NO: 513

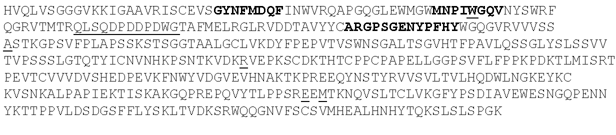

SEQ ID NO: 514
CACGTCCAATTGGTGCAGTCTGGAGGTGGGGTGAAGAAGATTGGGGCCGCTGTGAGGATCTCCTG
CGAGGTGTCTGGATACAACTTCATGGACCAATTCATAAATTGGGTGCGACAGGCCCCCGGTCAGG
GCCTTGAGTGGATGGGATGGATGAACCCAATATGGGGACAAGTAAATTATTCATGGAGATTTCAA
GGAAGGGTCACCATGACCAGGCAATTATCTCAAGACCCAGACGACCCGGACTGGGGCACGG
CCTTCATGGAGTTGAGAGGACTGAGAGTGGACGACACGGCCGTCTATTATTGCGCGAGGGGACCC
TCTGGGGAAAATTATCCTTTTCACTATTGGGGCCAGGGTGTCCGAGTGGTCGTCTCGTCAGCGTCC
ACCAAGGGCCCATCGGTCTTCCCCCTGGCACCCTCCTCCAAGAGCACCTCTGGGGGCACAGCGGC
CCTGGGCTGCCTGGTCAAGGACTACTTCCCCGAACCGGTGACGGTGTCGTGGAACTCAGGCGCCC
TGACCAGCGGCGTGCACACCTTCCCGGCTGTCCTACAGTCCTCAGGACTCTACTCCCTCAGCAGCG
TGGTGACCGTGCCCTCCAGCAGCTTGGGCACCCAGACCTACATCTGCAACGTGAATCACAAGCCC
AGCAACACCAAGGTGGACAAGAGAGTTGAGCCCAAATCTTGTGACAAAACTCACACATGCCCACC
GTGCCCAGCACCTGAACTCCTGGGGGGACCGTCAGTCTTCCTCTTCCCCCCAAAACCCAAGGACAC
CCTCATGATCTCCCGGACCCCTGAGGTCACATGCGTGGTGGTGGACGTGAGCCACGAAGACCCTG
AGGTCAAGTTCAACTGGTACGTGGACGGCGTGGAGGTGCATAATGCCAAGACAAAGCCGCGGGA
GGAGCAGTACAACAGCACGTACCGGGTGGTCAGCGTCCTCACCGTCCTGCACCAGGACTGGCTGA
ATGGCAAGGAGTACAAGTGCAAGGTCTCCAACAAAGCCCTCCCAGCCCCCATCGAGAAAACCATC
TCCAAAGCCAAAGGGCAGCCCCGAGAACCACAGGTGTACACCCTGCCCCCATCCCGGGAGGAGAT
GACCAAGAACCAGGTCAGCCTGACCTGCCTGGTCAAAGGCTTCTATCCCAGCGACATCGCCGTGG
AGTGGGAGAGCAATGGGCAGCCGGAGAACAACTACAAGACCACGCCTCCCGTGCTGGACTCCGA
CGGCTCCTTCTTCCTCTACAGCAAGCTCACCGTGGACAAGAGCAGGTGGCAGCAGGGGAACGTCT
TCTCATGCTCCGTGATGCATGAGGCTCTGCACAACCACTACACGCAGAAGAGCCTCTCCCTGTCTC
CGGGTAAA
LIGHT CHAIN
SEQ ID NO: 515

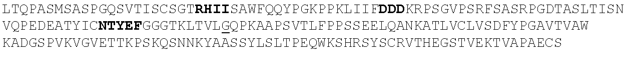

SEQ ID NO: 516
CTGACTCAGCCTGCCTCCATGTCTGCGTCCCCTGGACAGTCGGTAACCATCTCGTGCTCTGGAACC
AGACACATAATCTCTGCTTGGTTCCAACAATATCCAGGCAAACCACCCAAACTCATAATTTTTGAC
GACGATAAGCGTCCCTCTGGAGTTCCTAGTCGCTTCTCTGCCTCCAGGCCTGGCGACACGGCCTCC
CTGACAATCTCTAATGTTCAACCTGAGGACGAGGCGACATACATTTGCAATACATATGAATTCTTT
GGCGGAGGGACCAAATTGACCGTCCTAGGTCAGCCCAAGGCTGCCCCCTCGGTCACTCTGTTCCC
ACCCTCCTCTGAGGAGCTTCAAGCCAACAAGGCCACACTGGTGTGTCTCGTAAGTGACTTCTACCC
GGGAGCCGTGACAGTGGCCTGGAAGGCAGATGGCAGCCCCGTCAAGGTGGGAGTGGAGACCACC
AAACCCTCCAAACAAAGCAACAACAAGTATGCGGCCAGCAGCTACCTGAGCCTGACGCCCGAGC
AGTGGAAGTCCCACAGAAGCTACAGCTGCCGGGTCACGCATGAAGGGAGCACCGTGGAGAAGAC
AGTGGCCCCTGCAGAATGCTCT
>N49P9.6-FR-54F
HEAVY CHAIN
SEQ ID NO: 517

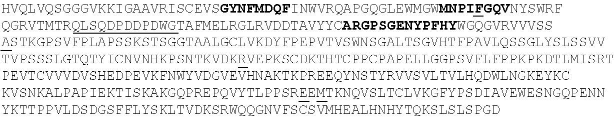

SEQ ID NO: 518
CACGTCCAATTGGTGCAGTCTGGAGGTGGGGTGAAGAAGATTGGGGCCGCTGTGAGGATCTCCTG
CGAGGTGTCTGGATACAACTTCATGGACCAATTCATAAATTGGGTGCGACAGGCCCCCGGTCAGG
GCCTTGAGTGGATGGGATGGATGAACCCAATATTTGGACAAGTAAATTATTCATGGAGATTTCAA
GGAAGGGTCACCATGACCAGGCAATTATCTCAAGACCCAGACGACCCGGACTGGGGCACGG
CCTTCATGGAGTTGAGAGGACTGAGAGTGGACGACACGGCCGTCTATTATTGCGCGAGGGGACCC
TCTGGGGAAAATTATCCTTTTCACTATTGGGGCCAGGGTGTCCGAGTGGTCGTCTCGTCAGCGTCC
ACCAAGGGCCCATCGGTCTTCCCCCTGGCACCCTCCTCCAAGAGCACCTCTGGGGGCACAGCGGC
CCTGGGCTGCCTGGTCAAGGACTACTTCCCCGAACCGGTGACGGTGTCGTGGAACTCAGGCGCCC
TGACCAGCGGCGTGCACACCTTCCCGGCTGTCCTACAGTCCTCAGGACTCTACTCCCTCAGCAGCG
TGGTGACCGTGCCCTCCAGCAGCTTGGGCACCCAGACCTACATCTGCAACGTGAATCACAAGCCC
AGCAACACCAAGGTGGACAAGAGAGTTGAGCCCAAATCTTGTGACAAAACTCACACATGCCCACC
GTGCCCAGCACCTGAACTCCTGGGGGGACCGTCAGTCTTCCTCTTCCCCCCAAAACCCAAGGACAC
CCTCATGATCTCCCGGACCCCTGAGGTCACATGCGTGGTGGTGGACGTGAGCCACGAAGACCCTG
AGGTCAAGTTCAACTGGTACGTGGACGGCGTGGAGGTGCATAATGCCAAGACAAAGCCGCGGGA
GGAGCAGTACAACAGCACGTACCGGGTGGTCAGCGTCCTCACCGTCCTGCACCAGGACTGGCTGA
ATGGCAAGGAGTACAAGTGCAAGGTCTCCAACAAAGCCCTCCCAGCCCCCATCGAGAAAACCATC
TCCAAAGCCAAAGGGCAGCCCCGAGAACCACAGGTGTACACCCTGCCCCCATCCCGGGAGGAGAT
GACCAAGAACCAGGTCAGCCTGACCTGCCTGGTCAAAGGCTTCTATCCCAGCGACATCGCCGTGG
AGTGGGAGAGCAATGGGCAGCCGGAGAACAACTACAAGACCACGCCTCCCGTGCTGGACTCCGA
CGGCTCCTTCTTCCTCTACAGCAAGCTCACCGTGGACAAGAGCAGGTGGCAGCAGGGGAACGTCT
TCTCATGCTCCGTGATGCATGAGGCTCTGCACAACCACTACACGCAGAAGAGCCTCTCCCTGTCTC
CGGGTAAA
LIGHT CHAIN
SEQ ID NO: 519

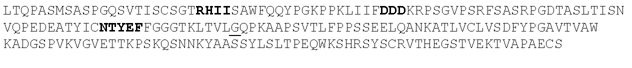

SEQ ID NO: 520
CTGACTCAGCCTGCCTCCATGTCTGCGTCCCCTGGACAGTCGGTAACCATCTCGTGCTCTGGAACC
AGACACATAATCTCTGCTTGGTTCCAACAATATCCAGGCAAACCACCCAAACTCATAATTTTTGAC
GACGATAAGCGTCCCTCTGGAGTTCCTAGTCGCTTCTCTGCCTCCAGGCCTGGCGACACGGCCTCC
CTGACAATCTCTAATGTTCAACCTGAGGACGAGGCGACATACATTTGCAATACATATGAATTCTTT
GGCGGAGGGACCAAATTGACCGTCCTAGGTCAGCCCAAGGCTGCCCCCTCGGTCACTCTGTTCCC
ACCCTCCTCTGAGGAGCTTCAAGCCAACAAGGCCACACTGGTGTGTCTCGTAAGTGACTTCTACCC
GGGAGCCGTGACAGTGGCCTGGAAGGCAGATGGCAGCCCCGTCAAGGTGGGAGTGGAGACCACC
AAACCCTCCAAACAAAGCAACAACAAGTATGCGGCCAGCAGCTACCTGAGCCTGACGCCCGAGC
AGTGGAAGTCCCACAGAAGCTACAGCTGCCGGGTCACGCATGAAGGGAGCACCGTGGAGAAGAC
AGTGGCCCCTGCAGAATGCTCT
>N49P9.6-FR3-06
HEAVY CHAIN
SEQ ID NO: 521

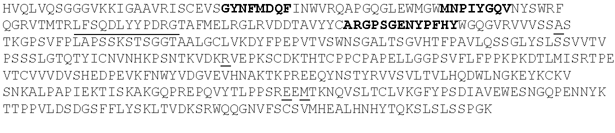

SEQ ID NO: 522
CACGTCCAATTGGTGCAGTCTGGAGGTGGGGTGAAGAAGATTGGGGCCGCTGTGAGGATCTCCTG
CGAGGTGTCTGGATACAACTTCATGGACCAATTCATAAATTGGGTGCGACAGGCCCCCGGTCAGG
GCCTTGAGTGGATGGGATGGATGAACCCAATATATGGACAAGTAAATTATTCATGGAGATTTCAA
GGAAGGGTCACCATGACCAGGCTATTCTCTCAAGACCTATACTACCCGGACCGGGGCACGGCCTT
CATGGAGTTGAGAGGACTGAGAGTGGACGACACGGCCGTCTATTATTGCGCGAGGGGACCCTCTG
GGGAAAATTATCCTTTTCACTATTGGGGCCAGGGTGTCCGAGTGGTCGTCTCGTCAGCGTCCACCA
AGGGCCCATCGGTCTTCCCCCTGGCACCCTCCTCCAAGAGCACCTCTGGGGGCACAGCGGCCCTG
GGCTGCCTGGTCAAGGACTACTTCCCCGAACCGGTGACGGTGTCGTGGAACTCAGGCGCCCTGAC
CAGCGGCGTGCACACCTTCCCGGCTGTCCTACAGTCCTCAGGACTCTACTCCCTCAGCAGCGTGGT
GACCGTGCCCTCCAGCAGCTTGGGCACCCAGACCTACATCTGCAACGTGAATCACAAGCCCAGCA
ACACCAAGGTGGACAAGAGAGTTGAGCCCAAATCTTGTGACAAAACTCACACATGCCCACCGTGC
CCAGCACCTGAACTCCTGGGGGGACCGTCAGTCTTCCTCTTCCCCCCAAAACCCAAGGACACCCTC
ATGATCTCCCGGACCCCTGAGGTCACATGCGTGGTGGTGGACGTGAGCCACGAAGACCCTGAGGT
CAAGTTCAACTGGTACGTGGACGGCGTGGAGGTGCATAATGCCAAGACAAAGCCGCGGGAGGAG
CAGTACAACAGCACGTACCGGGTGGTCAGCGTCCTCACCGTCCTGCACCAGGACTGGCTGAATGG
CAAGGAGTACAAGTGCAAGGTCTCCAACAAAGCCCTCCCAGCCCCCATCGAGAAAACCATCTCCA
AAGCCAAAGGGCAGCCCCGAGAACCACAGGTGTACACCCTGCCCCCATCCCGGGAGGAGATGAC
CAAGAACCAGGTCAGCCTGACCTGCCTGGTCAAAGGCTTCTATCCCAGCGACATCGCCGTGGAGT
GGGAGAGCAATGGGCAGCCGGAGAACAACTACAAGACCACGCCTCCCGTGCTGGACTCCGACGG
CTCCTTCTTCCTCTACAGCAAGCTCACCGTGGACAAGAGCAGGTGGCAGCAGGGGAACGTCTTCTC
ATGCTCCGTGATGCATGAGGCTCTGCACAACCACTACACGCAGAAGAGCCTCTCCCTGTCTCCGGG
TAAA
LIGHT CHAIN
SEQ ID NO: 523

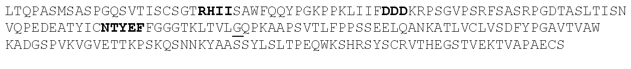

SEQ ID NO: 524
CTGACTCAGCCTGCCTCCATGTCTGCGTCCCCTGGACAGTCGGTAACCATCTCGTGCTCTGGAACC
AGACACATAATCTCTGCTTGGTTCCAACAATATCCAGGCAAACCACCCAAACTCATAATTTTTGAC
GACGATAAGCGTCCCTCTGGAGTTCCTAGTCGCTTCTCTGCCTCCAGGCCTGGCGACACGGCCTCC
CTGACAATCTCTAATGTTCAACCTGAGGACGAGGCGACATACATTTGCAATACATATGAATTCTTT
GGCGGAGGGACCAAATTGACCGTCCTAGGTCAGCCCAAGGCTGCCCCCTCGGTCACTCTGTTCCC
ACCCTCCTCTGAGGAGCTTCAAGCCAACAAGGCCACACTGGTGTGTCTCGTAAGTGACTTCTACCC
GGGAGCCGTGACAGTGGCCTGGAAGGCAGATGGCAGCCCCGTCAAGGTGGGAGTGGAGACCACC
AAACCCTCCAAACAAAGCAACAACAAGTATGCGGCCAGCAGCTACCTGAGCCTGACGCCCGAGC
AGTGGAAGTCCCACAGAAGCTACAGCTGCCGGGTCACGCATGAAGGGAGCACCGTGGAGAAGAC
AGTGGCCCCTGCAGAATGCTCT
>N49P9.6-FR1-D
HEAVY CHAIN
SEQ ID NO: 525

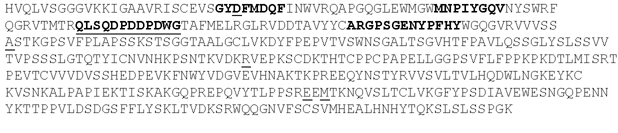

SEQ ID NO: 526
CACGTCCAATTGGTGCAGTCTGGAGGTGGGGTGAAGAAGATTGGGGCCGCTGTGAGGATCTCCTG
CGAGGTGTCTGGATACGACTTCATGGACCAATTCATAAATTGGGTGCGACAGGCCCCCGGTCAGG
GCCTTGAGTGGATGGGATGGATGAACCCAATATATGGACAAGTAAATTATTCATGGAGATTTCAA
GGAAGGGTCACCATGACCAGGCAATTATCTCAAGACCCAGACGACCCGGACTGGGGCACGG
CCTTCATGGAGTTGAGAGGACTGAGAGTGGACGACACGGCCGTCTATTATTGCGCGAGGGGACCC
TCTGGGGAAAATTATCCTTTTCACTATTGGGGCCAGGGTGTCCGAGTGGTCGTCTCGTCAGCGTCC
ACCAAGGGCCCATCGGTCTTCCCCCTGGCACCCTCCTCCAAGAGCACCTCTGGGGGCACAGCGGC
CCTGGGCTGCCTGGTCAAGGACTACTTCCCCGAACCGGTGACGGTGTCGTGGAACTCAGGCGCCC
TGACCAGCGGCGTGCACACCTTCCCGGCTGTCCTACAGTCCTCAGGACTCTACTCCCTCAGCAGCG
TGGTGACCGTGCCCTCCAGCAGCTTGGGCACCCAGACCTACATCTGCAACGTGAATCACAAGCCC
AGCAACACCAAGGTGGACAAGAGAGTTGAGCCCAAATCTTGTGACAAAACTCACACATGCCCACC
GTGCCCAGCACCTGAACTCCTGGGGGGACCGTCAGTCTTCCTCTTCCCCCCAAAACCCAAGGACAC
CCTCATGATCTCCCGGACCCCTGAGGTCACATGCGTGGTGGTGGACGTGAGCCACGAAGACCCTG
AGGTCAAGTTCAACTGGTACGTGGACGGCGTGGAGGTGCATAATGCCAAGACAAAGCCGCGGGA
GGAGCAGTACAACAGCACGTACCGGGTGGTCAGCGTCCTCACCGTCCTGCACCAGGACTGGCTGA
ATGGCAAGGAGTACAAGTGCAAGGTCTCCAACAAAGCCCTCCCAGCCCCCATCGAGAAAACCATC
TCCAAAGCCAAAGGGCAGCCCCGAGAACCACAGGTGTACACCCTGCCCCCATCCCGGGAGGAGAT
GACCAAGAACCAGGTCAGCCTGACCTGCCTGGTCAAAGGCTTCTATCCCAGCGACATCGCCGTGG
AGTGGGAGAGCAATGGGCAGCCGGAGAACAACTACAAGACCACGCCTCCCGTGCTGGACTCCGA
CGGCTCCTTCTTCCTCTACAGCAAGCTCACCGTGGACAAGAGCAGGTGGCAGCAGGGGAACGTCT
TCTCATGCTCCGTGATGCATGAGGCTCTGCACAACCACTACACGCAGAAGAGCCTCTCCCTGTCTC
CGGGTAAA
LIGHT CHAIN
SEQ ID NO: 572

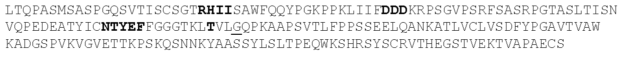

SEQ ID NO: 528
CTGACTCAGCCTGCCTCCATGTCTGCGTCCCCTGGACAGTCGGTAACCATCTCGTGCTCTGGAACC
AGACACATAATCTCTGCTTGGTTCCAACAATATCCAGGCAAACCACCCAAACTCATAATTTTTGAC
GACGATAAGCGTCCCTCTGGAGTTCCTAGTCGCTTCTCTGCCTCCAGGCCTGGCGACACGGCCTCC
CTGACAATCTCTAATGTTCAACCTGAGGACGAGGCGACATACATTTGCAATACATATGAATTCTTT
GGCGGAGGGACCAAATTGACCGTCCTAGGTCAGCCCAAGGCTGCCCCCTCGGTCACTCTGTTCCC
ACCCTCCTCTGAGGAGCTTCAAGCCAACAAGGCCACACTGGTGTGTCTCGTAAGTGACTTCTACCC
GGGAGCCGTGACAGTGGCCTGGAAGGCAGATGGCAGCCCCGTCAAGGTGGGAGTGGAGACCACC
AAACCCTCCAAACAAAGCAACAACAAGTATGCGGCCAGCAGCTACCTGAGCCTGACGCCCGAGC
AGTGGAAGTCCCACAGAAGCTACAGCTGCCGGGTCACGCATGAAGGGAGCACCGTGGAGAAGAC
AGTGGCCCCTGCAGAATGCTCT
>N49P9.6-FR1-D-I
HEAVY CHAIN
SEQ ID NO: 529

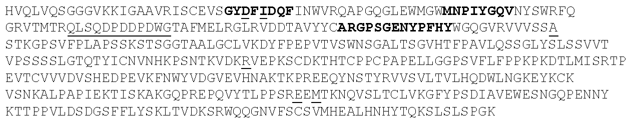

SEQ ID NO: 530
CACGTCCAATTGGTGCAGTCTGGAGGTGGGGTGAAGAAGATTGGGGCCGCTGTGAGGATCTCCTG
CGAGGTGTCTGGATACGACTTCATTGACCAATTCATAAATTGGGTGCGACAGGCCCCCGGTCAGG
GCCTTGAGTGGATGGGATGGATGAACCCAATATATGGACAAGTAAATTATTCATGGAGATTTCAA
GGAAGGGTCACCATGACCAGGCAATTATCTCAAGACCCAGACGACCCGGACTGGGGCACGG
CCTTCATGGAGTTGAGAGGACTGAGAGTGGACGACACGGCCGTCTATTATTGCGCGAGGGGACCC
TCTGGGGAAAATTATCCTTTTCACTATTGGGGCCAGGGTGTCCGAGTGGTCGTCTCGTCAGCGTCC
ACCAAGGGCCCATCGGTCTTCCCCCTGGCACCCTCCTCCAAGAGCACCTCTGGGGGCACAGCGGC
CCTGGGCTGCCTGGTCAAGGACTACTTCCCCGAACCGGTGACGGTGTCGTGGAACTCAGGCGCCC
TGACCAGCGGCGTGCACACCTTCCCGGCTGTCCTACAGTCCTCAGGACTCTACTCCCTCAGCAGCG
TGGTGACCGTGCCCTCCAGCAGCTTGGGCACCCAGACCTACATCTGCAACGTGAATCACAAGCCC
AGCAACACCAAGGTGGACAAGAGAGTTGAGCCCAAATCTTGTGACAAAACTCACACATGCCCACC
GTGCCCAGCACCTGAACTCCTGGGGGGACCGTCAGTCTTCCTCTTCCCCCCAAAACCCAAGGACAC
CCTCATGATCTCCCGGACCCCTGAGGTCACATGCGTGGTGGTGGACGTGAGCCACGAAGACCCTG
AGGTCAAGTTCAACTGGTACGTGGACGGCGTGGAGGTGCATAATGCCAAGACAAAGCCGCGGGA
GGAGCAGTACAACAGCACGTACCGGGTGGTCAGCGTCCTCACCGTCCTGCACCAGGACTGGCTGA
ATGGCAAGGAGTACAAGTGCAAGGTCTCCAACAAAGCCCTCCCAGCCCCCATCGAGAAAACCATC
TCCAAAGCCAAAGGGCAGCCCCGAGAACCACAGGTGTACACCCTGCCCCCATCCCGGGAGGAGAT
GACCAAGAACCAGGTCAGCCTGACCTGCCTGGTCAAAGGCTTCTATCCCAGCGACATCGCCGTGG
AGTGGGAGAGCAATGGGCAGCCGGAGAACAACTACAAGACCACGCCTCCCGTGCTGGACTCCGA
CGGCTCCTTCTTCCTCTACAGCAAGCTCACCGTGGACAAGAGCAGGTGGCAGCAGGGGAACGTCT
TCTCATGCTCCGTGATGCATGAGGCTCTGCACAACCACTACACGCAGAAGAGCCTCTCCCTGTCTC
CGGGTAAA
LIGHT CHAIN
SEQ ID NO: 531

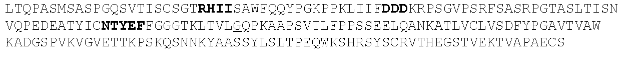

SEQ ID NO: 532
CTGACTCAGCCTGCCTCCATGTCTGCGTCCCCTGGACAGTCGGTAACCATCTCGTGCTCTGGAACC
AGACACATAATCTCTGCTTGGTTCCAACAATATCCAGGCAAACCACCCAAACTCATAATTTTTGAC
GACGATAAGCGTCCCTCTGGAGTTCCTAGTCGCTTCTCTGCCTCCAGGCCTGGCGACACGGCCTCC
CTGACAATCTCTAATGTTCAACCTGAGGACGAGGCGACATACATTTGCAATACATATGAATTCTTT
GGCGGAGGGACCAAATTGACCGTCCTAGGTCAGCCCAAGGCTGCCCCCTCGGTCACTCTGTTCCC
ACCCTCCTCTGAGGAGCTTCAAGCCAACAAGGCCACACTGGTGTGTCTCGTAAGTGACTTCTACCC
GGGAGCCGTGACAGTGGCCTGGAAGGCAGATGGCAGCCCCGTCAAGGTGGGAGTGGAGACCACC
AAACCCTCCAAACAAAGCAACAACAAGTATGCGGCCAGCAGCTACCTGAGCCTGACGCCCGAGC
AGTGGAAGTCCCACAGAAGCTACAGCTGCCGGGTCACGCATGAAGGGAGCACCGTGGAGAAGAC
AGTGGCCCCTGCAGAATGCTCT
>N49P9.6-FR-LS
HEAVY CHAIN
SEQ ID NO: 533

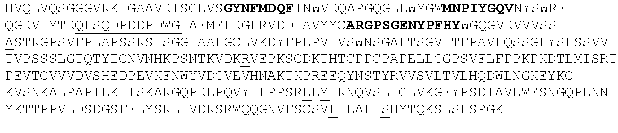

SEQ ID NO: 534
CACGTCCAATTGGTGCAGTCTGGAGGTGGGGTGAAGAAGATTGGGGCCGCTGTGAGGATCTCCTG
CGAGGTGTCTGGATACAACTTCATGGACCAATTCATAAATTGGGTGCGACAGGCCCCCGGTCAGG
GCCTTGAGTGGATGGGATGGATGAACCCAATATATGGACAAGTAAATTATTCATGGAGATTTCAA
GGAAGGGTCACCATGACCAGGCAATTATCTCAAGACCCAGACGACCCGGACTGGGGCACGG
CCTTCATGGAGTTGAGAGGACTGAGAGTGGACGACACGGCCGTCTATTATTGCGCGAGGGGACCC
TCTGGGGAAAATTATCCTTTTCACTATTGGGGCCAGGGTGTCCGAGTGGTCGTCTCGTCAGCGTCC
ACCAAGGGCCCATCGGTCTTCCCCCTGGCACCCTCCTCCAAGAGCACCTCTGGGGGCACAGCGGC
CCTGGGCTGCCTGGTCAAGGACTACTTCCCCGAACCGGTGACGGTGTCGTGGAACTCAGGCGCCC
TGACCAGCGGCGTGCACACCTTCCCGGCTGTCCTACAGTCCTCAGGACTCTACTCCCTCAGCAGCG
TGGTGACCGTGCCCTCCAGCAGCTTGGGCACCCAGACCTACATCTGCAACGTGAATCACAAGCCC
AGCAACACCAAGGTGGACAAGAGAGTTGAGCCCAAATCTTGTGACAAAACTCACACATGCCCACC
GTGCCCAGCACCTGAACTCCTGGGGGGACCGTCAGTCTTCCTCTTCCCCCCAAAACCCAAGGACAC
CCTCATGATCTCCCGGACCCCTGAGGTCACATGCGTGGTGGTGGACGTGAGCCACGAAGACCCTG
AGGTCAAGTTCAACTGGTACGTGGACGGCGTGGAGGTGCATAATGCCAAGACAAAGCCGCGGGA
GGAGCAGTACAACAGCACGTACCGGGTGGTCAGCGTCCTCACCGTCCTGCACCAGGACTGGCTGA
ATGGCAAGGAGTACAAGTGCAAGGTCTCCAACAAAGCCCTCCCAGCCCCCATCGAGAAAACCATC
TCCAAAGCCAAAGGGCAGCCCCGAGAACCACAGGTGTACACCCTGCCCCCATCCCGGGAGGAGAT
GACCAAGAACCAGGTCAGCCTGACCTGCCTGGTCAAAGGCTTCTATCCCAGCGACATCGCCGTGG
AGTGGGAGAGCAATGGGCAGCCGGAGAACAACTACAAGACCACGCCTCCCGTGCTGGACTCCGA
CGGCTCCTTCTTCCTCTACAGCAAGCTCACCGTGGACAAGAGCAGGTGGCAGCAGGGGAACGTCT
TCTCATGCTCCGTGCTGCATGAGGCTCTGCACAGCCACTACACGCAGAAGAGCCTCTCCCTGTCTC
CGGGTAAA
LIGHT CHAIN
SEQ ID NO: 535

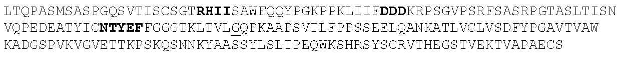

SEQ ID NO: 536
CTGACTCAGCCTGCCTCCATGTCTGCGTCCCCTGGACAGTCGGTAACCATCTCGTGCTCTGGAACC
AGACACATAATCTCTGCTTGGTTCCAACAATATCCAGGCAAACCACCCAAACTCATAATTTTTGAC
GACGATAAGCGTCCCTCTGGAGTTCCTAGTCGCTTCTCTGCCTCCAGGCCTGGCGACACGGCCTCC
CTGACAATCTCTAATGTTCAACCTGAGGACGAGGCGACATACATTTGCAATACATATGAATTCTTT
GGCGGAGGGACCAAATTGACCGTCCTAGGTCAGCCCAAGGCTGCCCCCTCGGTCACTCTGTTICCC
ACCCTCCTCTGAGGAGCTTCAAGCCAACAAGGCCACACTGGTGTGTCTCGTAAGTGACTTCTACCC
GGGAGCCGTGACAGTGGCCTGGAAGGCAGATGGCAGCCCCGTCAAGGTGGGAGTGGAGACCACC
AAACCCTCCAAACAAAGCAACAACAAGTATGCGGCCAGCAGCTACCTGAGCCTGACGCCCGAGC
AGTGGAAGTCCCACAGAAGCTACAGCTGCCGGGTCACGCATGAAGGGAGCACCGTGGAGAAGAC
AGTGGCCCCTGCAGAATGCTCT
>N49P9.6-FR-YTE
HEAVY CHAIN
SEQ ID NO: 537

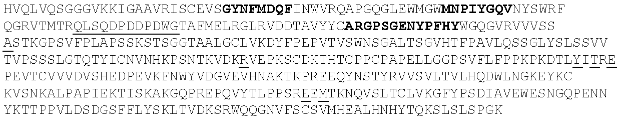

SEQ ID NO: 538
CACGTCCAATTGGTGCAGTCTGGAGGTGGGGTGAAGAAGATTGGGGCCGCTGTGAGGATCTCCTG
CGAGGTGTCTGGATACAACTTCATGGACCAATTCATAAATTGGGTGCGACAGGCCCCCGGTCAGG
GCCTTGAGTGGATGGGATGGATGAACCCAATATATGGACAAGTAAATTATTCATGGAGATTTCAA
GGAAGGGTCACCATGACCAGGCAATTATCTCAAGACCCAGACGACCCGGACTGGGGCACGG
CCTTCATGGAGTTGAGAGGACTGAGAGTGGACGACACGGCCGTCTATTATTGCGCGAGGGGACCC
TCTGGGGAAAATTATCCTTTTCACTATTGGGGCCAGGGTGTCCGAGTGGTCGTCTCGTCAGCGTCC
ACCAAGGGCCCATCGGTCTTCCCCCTGGCACCCTCCTCCAAGAGCACCTCTGGGGGCACAGCGGC
CCTGGGCTGCCTGGTCAAGGACTACTTCCCCGAACCGGTGACGGTGTCGTGGAACTCAGGCGCCC
TGACCAGCGGCGTGCACACCTTCCCGGCTGTCCTACAGTCCTCAGGACTCTACTCCCTCAGCAGCG
TGGTGACCGTGCCCTCCAGCAGCTTGGGCACCCAGACCTACATCTGCAACGTGAATCACAAGCCC
AGCAACACCAAGGTGGACAAGAGAGTTGAGCCCAAATCTTGTGACAAAACTCACACATGCCCACC
GTGCCCAGCACCTGAACTCCTGGGGGGACCGTCAGTCTTCCTCTTCCCCCCAAAACCCAAGGACAC
CCTCTATATCACCCGGGAGCCTGAGGTCACATGCGTGGTGGTGGACGTGAGCCACGAAGACCCTG
AGGTCAAGTTCAACTGGTACGTGGACGGCGTGGAGGTGCATAATGCCAAGACAAAGCCGCGGGA
GGAGCAGTACAACAGCACGTACCGGGTGGTCAGCGTCCTCACCGTCCTGCACCAGGACTGGCTGA
ATGGCAAGGAGTACAAGTGCAAGGTCTCCAACAAAGCCCTCCCAGCCCCCATCGAGAAAACCATC
TCCAAAGCCAAAGGGCAGCCCCGAGAACCACAGGTGTACACCCTGCCCCCATCCCGGGAGGAGAT
GACCAAGAACCAGGTCAGCCTGACCTGCCTGGTCAAAGGCTTCTATCCCAGCGACATCGCCGTGG
AGTGGGAGAGCAATGGGCAGCCGGAGAACAACTACAAGACCACGCCTCCCGTGCTGGACTCCGA
CGGCTCCTTCTTCCTCTACAGCAAGCTCACCGTGGACAAGAGCAGGTGGCAGCAGGGGAACGTCT
TCTCATGCTCCGTGATGCATGAGGCTCTGCACAACCACTACACGCAGAAGAGCCTCTCCCTGTCTC
CGGGTAAA
LIGHT CHAIN
SEQ ID NO: 539

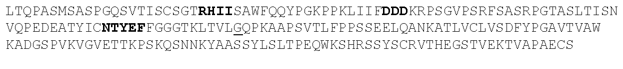

SEQ ID NO: 540
CTGACTCAGCCTGCCTCCATGTCTGCGTCCCCTGGACAGTCGGTAACCATCTCGTGCTCTGGAACC
AGACACATAATCTCTGCTTGGTTCCAACAATATCCAGGCAAACCACCCAAACTCATAATTTTTGAC
GACGATAAGCGTCCCTCTGGAGTTCCTAGTCGCTTCTCTGCCTCCAGGCCTGGCGACACGGCCTCC
CTGACAATCTCTAATGTTCAACCTGAGGACGAGGCGACATACATTTGCAATACATATGAATTCTTT
GGCGGAGGGACCAAATTGACCGTCCTAGGTCAGCCCAAGGCTGCCCCCTCGGTCACTCTGTTCCC
ACCCTCCTCTGAGGAGCTTCAAGCCAACAAGGCCACACTGGTGTGTCTCGTAAGTGACTTCTACCC
GGGAGCCGTGACAGTGGCCTGGAAGGCAGATGGCAGCCCCGTCAAGGTGGGAGTGGAGACCACC
AAACCCTCCAAACAAAGCAACAACAAGTATGCGGCCAGCAGCTACCTGAGCCTGACGCCCGAGC
AGTGGAAGTCCCACAGAAGCTACAGCTGCCGGGTCACGCATGAAGGGAGCACCGTGGAGAAGAC
AGTGGCCCCTGCAGAATGCTCT

The anti-HIV antibodies can also include insertions, deletions, substitutions, or other selected modifications of particular regions or specific amino acids residues. It should be understood that the antibodies may differ from the exact sequences illustrated and described herein. Thus, the invention contemplates antibodies that have deletions, additions and substitutions to the sequences shown, so long as the sequences function in accordance with the methods of the invention. In this regard, particularly preferred substitutions will generally be conservative in nature, i.e., those substitutions that take place within a family of amino acids. For example, amino acids are generally divided into four families: (1) acidic—aspartate and glutamate; (2) basic—lysine, arginine, histidine; (3) non-polar—alanine, valine, leucine, isoleucine, proline, phenylalanine, methionine, tryptophan; and (4) uncharged polar-glycine, asparagine, glutamine, cystine, serine threonine, tyrosine. Phenylalanine, tryptophan, and tyrosine are sometimes classified as aromatic amino acids. For example, leucine can be replaced with isoleucine or valine, or vice versa; an aspartate with a glutamate or vice versa; a threonine with a serine or vice versa; or a similar conservative replacement of an amino acid with a structurally related amino acid can be made.

In some embodiments, the anti-HIV antibody is an antibody that binds to the same epitope as an antibody selected from the group consisting of N49P7-FR, N49P9-FR, N49P9.3-FR, N49P9.6-FR, N49P9.6-FR-54W, N49P9.6-FR-54F, N49P9.6-FR3-06, N49P9.6-FR1-D, N49P9.6-FR1-D-I, N49P9.6, N49P9.6-54W, N49P9.6-54F, N49P9.6-LS, N49P9.6-YTE, N49P9.6-FR-LS, and N49P9.6-FR-YTE.

In some embodiments, the anti-HIV antibody comprises a heavy chain comprising an amino acid sequence selected from the group consisting of SEQ ID NOS:501, 505, 507, 509, 513, 517, 521, 525, 529, 533, and 537.

In some embodiments, the anti-HIV antibody comprises an antigen binding fragment of an amino acid sequence selected from the group consisting of SEQ ID NOS:501, 505, 507, 509, 513, 517, 521, 525, 529, 533, and 537.

In some embodiments, the anti-HIV antibody comprises a light chain comprising an amino acid sequence selected from the group consisting of SEQ ID NOS:503, 295, 327, 511, 515, 519, 523, 527, 531, 535, and 539.

In some embodiments, the anti-HIV antibody comprises an antigen binding fragment of an amino acid sequence selected from the group consisting of SEQ ID NOS:503, 295, 327, 511, 515, 519, 523, 527, 531, 535, and 539.

In some embodiments, the anti-HIV antibody comprises a heavy chain comprising an amino acid sequence selected from the group consisting of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 153, 157, 161, 165, 169, 173, 177, 181, 185, 189, 193, 197, 201, 205, 209, 213, 217, 221, 225, 229, 233, 237, 241, 245, 249, 253, 257, 261, 265, 269, 273, 277, 281, 285, 289, 293, 297, 301, 305, 309, 313, 317, 321, 325, 329, 333, 337, 341, 345, 349, 353, 357, 361, 365, 369, 373, 377, 381, 385, 389, 393 and 397. In some embodiments, the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542).

In some embodiments, the anti-HIV antibody comprises an antigen binding fragment of an amino acid sequence selected from the group consisting of SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 153, 157, 161, 165, 169, 173, 177, 181, 185, 189, 193, 197, 201, 205, 209, 213, 217, 221, 225, 229, 233, 237, 241, 245, 249, 253, 257, 261, 265, 269, 273, 277, 281, 285, 289, 293, 297, 301, 305, 309, 313, 317, 321, 325, 329, 333, 337, 341, 345, 349, 353, 357, 361, 365, 369, 373, 377, 381, 385, 389, 393 and 397. In some embodiments, the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542).

In some embodiments, the anti-HIV antibody comprises a light chain comprising an amino acid sequence selected from the group consisting of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 155, 159, 163, 167, 171, 175, 179, 183, 187, 191, 195, 199, 203, 207, 211, 215, 219, 223, 227, 231, 235, 239, 243, 247, 251, 255, 259, 263, 267, 271, 275, 279, 283, 287, 291, 295, 299, 303, 307, 311, 315, 319, 323, 327, 331, 335, 339, 343, 347, 351, 355, 359, 363, 367, 371, 375, 379, 383, 387, 391, 395, 399, 503, 511, 515, 519, 523, 527, 531, 535, and 539.

In some embodiments, the anti-HIV antibody comprises an antigen binding fragment of an amino acid sequence selected from the group consisting of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 155, 159, 163, 167, 171, 175, 179, 183, 187, 191, 195, 199, 203, 207, 211, 215, 219, 223, 227, 231, 235, 239, 243, 247, 251, 255, 259, 263, 267, 271, 275, 279, 283, 287, 291, 295, 299, 303, 307, 311, 315, 319, 323, 327, 331, 335, 339, 343, 347, 351, 355, 359, 363, 367, 371, 375, 379, 383, 387, 391, 395, 399, 503, 511, 515, 519, 523, 527, 531, 535, and 539.

For any of the light chain sequences described herein, and for any of the antibodies described herein, the first 1, 2, or 3 amino acids in the light chain can be deleted, or can be substituted with another amino acid, e.g., a conservative amino acid substitution. In some embodiments there is a deletion of the first 2 or 3 amino acids. In some embodiments, the antibodies can comprise the variable region of such antibodies. For example, the variable region of SEQ ID NO:36 comprises amino acids 1-100 of SEQ ID NO:36. If an antibody has a light chain that has a 2 amino acid deletion in SEQ ID NO:36, the variable region will comprise amino acids 3-100 of SEQ ID NO:36.

In some embodiments, the anti-HIV antibody is selected from the group consisting of:
a) an antibody comprising a heavy chain variable region, wherein CDR H1 comprises GYDFIDYV (SEQ ID NO:401), CDR H2 comprises MNPSGGGT (SEQ ID NO:402) and CDR H3 comprises VRDRANGSGRRRFESVNWFLDL (SEQ ID NO:403); and a light chain variable region, wherein CDR L1 comprises HNY, CDR L2 comprises DFN and CDR L3 comprises WAFEN (SEQ ID NO:404);
b) an antibody comprising a heavy chain variable region, wherein CDR H1 comprises GYKFPDYI (SEQ ID NO:405), CDR H2 comprises INPMGGQV (SEQ ID NO:406) and CDR H3 comprises VRDRSNGSGRRFESSN (SEQ ID NO:407); and a light chain variable region, wherein CDR L1 comprises HNL, CDR L2 comprises DFN and CDR L3 comprises WAYEA (SEQ ID NO:408);
c) an antibody comprising a heavy chain variable region, wherein CDR H1 comprises GYTFTDYL (SEQ ID NO:409), CDR H2 comprises MNPVYGQV (SEQ ID NO:410) and CDR H3 comprises VRDTGDGSRRHFDSINWFLDL (SEQ ID NO:411); and a light chain variable region, wherein CDR L1 comprises HNY, CDR L2 comprises DFD and CDR L3 comprises WAFEA (SEQ ID NO:412);
d) an antibody comprising a heavy chain variable region, wherein CDR H1 comprises GYTFTDYV (SEQ ID NO:413), CDR H2 comprises IDPPYGQV (SEQ ID NO:414) and CDR H3 comprises VRDRSNGWGKRFESSNWFLDL (SEQ ID NO:415); and a light chain variable region, wherein CDR L1 comprises HNL, CDR L2 comprises DFN and CDR L3 comprises WAYEA (SEQ ID NO:408);
e) an antibody comprising a heavy chain variable region, wherein CDR H1 comprises GYTFVDYF (SEQ ID NO:416), CDR H2 comprises MDPLNGRP (SEQ ID NO:417) and CDR H3 comprises VRDKSNGSGRRFDSSNWFLDL (SEQ ID NO:418); and a light chain variable region, wherein CDR L1 comprises HNY, CDR L2 comprises DFN and CDR L3 comprises WAYDA (SEQ ID NO:419);
f) an antibody comprising a heavy chain variable region, wherein CDR H1 comprises GYTFSDYI (SEQ ID NO:420), CDR H2 comprises MNPMGGQV (SEQ ID NO:421) and CDR H3 comprises VRDRSNGSGKRFESSNWFLDL (SEQ ID NO:441); and a light chain variable region, wherein CDR L1 comprises HNL, CDR L2 comprises DFN and CDR L3 comprises WAYEV (SEQ ID NO:422);
g. an antibody comprising a heavy chain variable region, wherein CDR H1 comprises GYTFIDYI (SEQ ID NO:423), CDR H2 comprises IDPMNGRP (SEQ ID NO:424) and CDR H3 comprises VRDKSNGSGKRFDSSNWFLDL (SEQ ID NO:425); and a light chain variable region, wherein CDR L1 comprises HNY, CDR L2 comprises DFN and CDR L3 comprises WAYDA (SEQ ID NO:419);
h. an antibody comprising a heavy chain variable region, wherein CDR H1 comprises GYTFTDYI (SEQ ID NO:426), CDR H2 comprises MNPMGGRT (SEQ ID NO:427) and CDR H3 comprises VRDKSNGSGKRFDSSNWFLDL (SEQ ID NO:425); and a light chain variable region, wherein CDR L1 comprises HNL, CDR L2 comprises DFN and CDR L3 comprises WAYEA (SEQ ID NO:408);
i. an antibody comprising a heavy chain variable region, wherein CDR H1 comprises GYTFVDYL (SEQ ID NO:428), CDR H2 comprises MDPMNGRP (SEQ ID NO:429) and CDR H3 comprises VRDKSGGSGKLFDSSNWFLDL (SEQ ID NO:430); and a light chain variable region, wherein CDR L1 comprises HNY, CDR L2 comprises DFN and CDR L3 comprises WAYDA (SEQ ID NO:419);
j. an antibody comprising a heavy chain variable region, wherein CDR H1 comprises GYTFTDYV (SEQ ID NO:413), CDR H2 comprises INPGYGQV (SEQ ID NO:431) and CDR H3 comprises VRDRSNGWGKRFESSNWFLDL (SEQ ID NO:415); and a light chain variable region, wherein CDR L1 comprises HNL, CDR L2 comprises DFN and CDR L3 comprises WAYEA (SEQ ID NO:408);
k. an antibody comprising a heavy chain variable region, wherein CDR H1 comprises GYTFTDYV (SEQ ID NO:413), CDR H2 comprises MDPSYGQV (SEQ ID NO:432) and CDR H3 comprises VRDRSHGSGRQFESSNWFLDL (SEQ ID NO:433); and a light chain variable region, wherein CDR L1 comprises HNL, CDR L2 comprises DFN and CDR L3 comprises WAYEA (SEQ ID NO:408);
l. an antibody comprising a heavy chain variable region, wherein CDR H1 comprises GYTFTDYV (SEQ ID NO:413), CDR H2 comprises MDPSFGQM (SEQ ID NO:434) and CDR H3 comprises VRDRSHGSGRLFESSNWFLDL (SEQ ID NO:435); and a light chain variable region, wherein CDR L1 comprises HNL, CDR L2 comprises DFN and CDR L3 comprises WAYEA (SEQ ID NO:408);
m. an antibody comprising a heavy chain variable region, wherein CDR H1 comprises GYRFTDYV (SEQ ID NO:436), CDR H2 comprises MDPSFGRM (SEQ ID NO:437) and CDR H3 comprises VRDRSHGSGRLFESSNWFLDL (SEQ ID NO:435); and a light chain variable region, wherein CDR L1 comprises HNL, CDR L2 comprises DFN and CDR L3 comprises WAYEA (SEQ ID NO:408);
n. an antibody comprising a heavy chain variable region, wherein CDR H1 comprises GYTFIDYV (SEQ ID NO:438), CDR H2 comprises MDPTYGRM (SEQ ID NO:439) and CDR H3 comprises VRDRSHGSGRLFESSNWFLDL (SEQ ID NO:435); and a light chain variable region, wherein CDR L1 comprises HNL, CDR L2 comprises DFN and CDR L3 comprises WAYEA (SEQ ID NO:408);
o. an antibody comprising a heavy chain variable region, wherein CDR H1 comprises GYRFLDYI (SEQ ID NO:440), CDR H2 comprises MNPMGGQV (SEQ ID NO:421) and CDR H3 comprises VRDRSNGSGKRFESSNWFLDL (SEQ ID NO:441); and a light chain variable region, wherein CDR L1 comprises HNL, CDR L2 comprises DFN and CDR L3 comprises WAYEA (SEQ ID NO:408);
p. an antibody comprising a heavy chain variable region, wherein CDR H1 comprises GYKFMDQL (SEQ ID NO:442), CDR H2 comprises MNPTYGQV (SEQ ID NO:443) and CDR H3 comprises ARGPSGENYPFHY (SEQ ID NO:444); and a light chain variable region, wherein CDR L1 comprises RHII (SEQ ID NO:445), CDR L2 comprises DDD and CDR L3 comprises NTYEF (SEQ ID NO:446);
q. an antibody comprising a heavy chain variable region, wherein CDR H1 comprises GYNFVDSR (SEQ ID NO:447), CDR H2 comprises INPLQGGV (SEQ ID NO:448) and CDR H3 comprises ARGIDGKSYPFHF (SEQ ID NO:449); and a light chain variable region, wherein CDR L1 comprises S, CDR L2 comprises ESS and CDR L3 comprises SILEF (SEQ ID NO:450);
r. an antibody comprising a heavy chain variable region, wherein CDR H1 comprises GYTFTTHHGHF (SEQ ID NO:500), CDR H2 comprises MNPMTGQM (SEQ ID NO:462) and CDR H3 comprises ARGDFGQNYPFHY (SEQ ID NO:463); and a light chain variable region, wherein CDR L1 comprises NRYL (SEQ ID NO:464), CDR L2 comprises DDN and CDR L3 comprises ASYER (SEQ ID NO:465);
s. an antibody comprising a heavy chain variable region, wherein CDR H1 comprises GYNFMDQF (SEQ ID NO:466), CDR H2 comprises MNPIYGQV (SEQ ID NO:467) and CDR H3 comprises ARGPSGENYPFHY (SEQ ID NO:444); and a light chain variable region, wherein CDR L1 comprises RHII (SEQ ID NO:445), CDR L2 comprises DDD and CDR L3 comprises NTYEF (SEQ ID NO:446);
t. an antibody comprising a heavy chain variable region, wherein CDR H1 comprises GYNFVDSR (SEQ ID NO:447), CDR H2 comprises INPLHGGV (SEQ ID NO:468) and CDR H3 comprises ARGIDGKSYPFHF (SEQ ID NO:449); and a light chain variable region, wherein CDR L1 comprises S, CDR L2 comprises ESS and CDR L3 comprises SILEF (SEQ ID NO:450);
u. an antibody comprising a heavy chain variable region, wherein CDR H1 comprises GYTFTKYF (SEQ ID NO:451), CDR H2 comprises IHPRTGAV (SEQ ID NO:452) and CDR H3 comprises ARGAFEADSYGSSYPFHH (SEQ ID NO:453); and a light chain variable region, wherein CDR L1 comprises GNYNP (SEQ ID NO:454), CDR L2 comprises EDN and CDR L3 comprises ASFEF (SEQ ID NO:455);
v. an antibody comprising a heavy chain variable region, wherein CDR H1 comprises GYTFTKYT (SEQ ID NO:456), CDR H2 comprises IHPRTGAV (SEQ ID NO:452) and CDR H3 comprises ARGAFEADLSGPTYPFHH (SEQ ID NO:457); and a light chain variable region, wherein CDR L1 comprises GNYNP (SEQ ID NO:454), CDR L2 comprises EDN and CDR L3 comprises ASFEF (SEQ ID NO:455);
w. an antibody comprising a heavy chain variable region, wherein CDR H1 comprises GFNFIDSV (SEQ ID NO:458), CDR H2 comprises IKPLRGAV (SEQ ID NO:459) and CDR H3 comprises AKGAFRGGSPFGF (SEQ ID NO:460); and a light chain variable region, wherein CDR L1 comprises DVT and CDR L2 comprises ASREF (SEQ ID NO:461);
x. an antibody comprising a heavy chain variable region, wherein CDR H1 comprises GYTFTSYF (SEQ ID NO:469), CDR H2 comprises INPLHGAV (SEQ ID NO:470) and CDR H3 comprises TRGIVADGWPYGH (SEQ ID NO:471); and a light chain variable region, wherein CDR L1 comprises S, CDR L2 comprises EGA and CDR L3 comprises SSLQF (SEQ ID NO:472);
y. an antibody comprising a heavy chain variable region, wherein CDR H1 comprises GFTFIDHI (SEQ ID NO:473), CDR H2 comprises IKPLRGAV (SEQ ID NO:459) and CDR H3 comprises CKAAAPEEAFPLQY (SEQ ID NO:474); and a light chain variable region, wherein CDR L1 comprises NVD, CDR L2 comprises DNN and CDR L3 comprises SSRTF (SEQ ID NO:475);
z. an antibody comprising a heavy chain variable region, wherein CDR H1 comprises GFKFIDHI (SEQ ID NO:476), CDR H2 comprises IKPLGGVA (SEQ ID NO:477) and CDR H3 comprises CKAAAPDEAFPLEY (SEQ ID NO:478); and a light chain variable region, wherein CDR L1 comprises NVD, CDR L2 comprises DNN and CDR L3 comprises SSTTF (SEQ ID NO:479);
aa. an antibody comprising a heavy chain variable region, wherein CDR H1 comprises GFAFLDH (SEQ ID NO:480), CDR H2 comprises VKTIGGVV (SEQ ID NO:481) and CDR H3 comprises SKAAAPDEAFPLEF (SEQ ID NO:482); and a light chain variable region, wherein CDR L1 comprises NVD, CDR L2 comprises DNN and CDR L3 comprises SSTTF (SEQ ID NO:479);
bb. an antibody comprising a heavy chain variable region, wherein CDR H1 comprises GFKFTEYF (SEQ ID NO:483), CDR H2 comprises LNPLRGAV (SEQ ID NO:484) and CDR H3 comprises ARAVFNEAFPFDY (SEQ ID NO:485); and a light chain variable region, wherein CDR L1 comprises VS, CDR L2 comprises DGD and CDR L3 comprises ASREF (SEQ ID NO:461);
cc. an antibody comprising a heavy chain variable region, wherein CDR H1 comprises GFKFIDHI (SEQ ID NO:476), CDR H2 comprises IKPLGGVA (SEQ ID NO:477) and CDR H3 comprises CKAAAPDEAFPLEY (SEQ ID NO:478); and a light chain variable region, wherein CDR L1 comprises NVD, CDR L2 comprises DND and CDR L3 comprises SSTTF (SEQ ID NO:479);
dd. an antibody comprising a heavy chain variable region, wherein CDR H1 comprises GFAFLDHI (SEQ ID NO:486), CDR H2 comprises VKTIGGVV (SEQ ID NO:481) and CDR H3 comprises SKAAAPDEAFPLEF (SEQ ID NO:482); and a light chain variable region, wherein CDR L1 comprises NVD, CDR L2 comprises DNN and CDR L3 comprises SSTTF (SEQ ID NO:479);
ee. an antibody comprising a heavy chain variable region, wherein CDR H1 comprises GFKFIDSV (SEQ ID NO:487), CDR H2 comprises IKPLGGAV (SEQ ID NO:488) and CDR H3 comprises AKGAFGGGSPFGF (SEQ ID NO:489); and a light chain variable region, wherein CDR L1 comprises DVT and CDR L2 comprises ASREF (SEQ ID NO:461);
ff. an antibody comprising a heavy chain variable region, wherein CDR H1 comprises GFNFIDSV (SEQ ID NO:458), CDR H2 comprises IKPLRGGV (SEQ ID NO:490) and CDR H3 comprises AKGAFGGSSPFGF (SEQ ID NO:491); and a light chain variable region, wherein CDR L1 comprises DVT and CDR L2 comprises ASREF (SEQ ID NO:461);
gg. an antibody comprising a heavy chain variable region, wherein CDR H1 comprises GFTFIKYT (SEQ ID NO:492), CDR H2 comprises IHPRTGAV (SEQ ID NO:452) and CDR H3 comprises ARGAFEADLYGPTYPFHH (SEQ ID NO:493); and a light chain variable region, wherein CDR L1 comprises GSYNP (SEQ ID NO:494), CDR L2 comprises DDN and CDR L3 comprises ASFEF (SEQ ID NO:455);
hh. an antibody comprising a heavy chain variable region, wherein CDR H1 comprises GYNFVDSL (SEQ ID NO:495), CDR H2 comprises INPLQGGV (SEQ ID NO:448) and CDR H3 comprises ARGIDGNSYPFHF (SEQ ID NO:496); and a light chain variable region, wherein CDR L1 comprises S, CDR L2 comprises ESS and CDR L3 comprises SILEF (SEQ ID NO:450);
ii. an antibody comprising a heavy chain variable region, wherein CDR H1 comprises GYRFPDYI (SEQ ID NO:497), CDR H2 comprises MNPTYGQV (SEQ ID NO:443) and CDR H3 comprises VRDRSNGSGKRFESSNWFLDL (SEQ ID NO:441); and a light chain variable region, wherein CDR L1 comprises HNL, CDR L2 comprises DFN and CDR L3 comprises WAYEA (SEQ ID NO:408);
jj. an antibody comprising a heavy chain variable region, wherein CDR H1 comprises GYRFPDYI (SEQ ID NO:497), CDR H2 comprises MNPMGGQV (SEQ ID NO:421) and CDR H3 comprises VRDRSNGSGKRFESSNWFLDL (SEQ ID NO:441); and a light chain variable region, wherein CDR L1 comprises HNY, CDR L2 comprises DFN and CDR L3 comprises WAFEN (SEQ ID NO:404);
kk. an antibody comprising a heavy chain variable region, wherein CDR H1 comprises GYRFPDYI (SEQ ID NO:497), CDR H2 comprises MNPMGGQV (SEQ ID NO:421) and CDR H3 comprises VRDRSNGSGKRFESSNWFLDL (SEQ ID NO:441); and a light chain variable region, wherein CDR L1 comprises HNY, CDR L2 comprises DFD and CDR L3 comprises WAFEA (SEQ ID NO:412);
ll. an antibody comprising a heavy chain variable region, wherein CDR H1 comprises GYRFPDYI (SEQ ID NO:497), CDR H2 comprises MNPMGGQV (SEQ ID NO:421) and CDR H3 comprises VRDRSNGSGKRFESSNWFLDL (SEQ ID NO:441); and a light chain variable region, wherein CDR L1 comprises RHII (SEQ ID NO:445), CDR L2 comprises DDD and CDR L3 comprises NTYEF (SEQ ID NO:446);
mm. an antibody comprising a heavy chain variable region, wherein CDR H1 comprises GYRFPDYI (SEQ ID NO:497), CDR H2 comprises MNPMGGQV (SEQ ID NO:421) and CDR H3 comprises VRDRSNGSGKRFESSNWFLDL (SEQ ID NO:441); and a light chain variable region, wherein CDR L1 comprises HNY, CDR L2 comprises DFN and CDR L3 comprises WAYDA (SEQ ID NO:419);
nn. an antibody comprising a heavy chain variable region, wherein CDR H1 comprises GYRFPDYI (SEQ ID NO:497), CDR H2 comprises MNPMGGQV (SEQ ID NO:421) and CDR H3 comprises VRDRSNGSGKRFESSNWFLDL (SEQ ID NO:441); and a light chain variable region, wherein CDR L1 comprises HNL, CDR L2 comprises DFN and CDR L3 comprises WAYEA (SEQ ID NO:408);
oo. an antibody comprising a heavy chain variable region, wherein CDR H1 comprises GYKFMDQL (SEQ ID NO:442), CDR H2 comprises MNPTYGQV (SEQ ID NO:443) and CDR H3 comprises VRDRSNGSGKRFESSN (SEQ ID NO:498); and a light chain variable region, wherein CDR L1 comprises RHII (SEQ ID NO:445), CDR L2 comprises DDD and CDR L3 comprises NTYEF (SEQ ID NO:446);
pp. an antibody comprising a heavy chain variable region, wherein CDR H1 comprises GYKFMDQL (SEQ ID NO:442), CDR H2 comprises (SEQ ID NO:443) and CDR H3 comprises VRDRSNGSGKRFESSNWFLDL (SEQ ID NO:441); and a light chain variable region, wherein CDR L1 comprises RHII (SEQ ID NO:445), CDR L2 comprises DDD and CDR L3 comprises NTYEF (SEQ ID NO:446); and
qq. an antibody comprising a heavy chain variable region, wherein CDR H1 comprises GYRFLDYI (SEQ ID NO:440), CDR H2 comprises MNPMGGQV (SEQ ID NO:421) and CDR H3 comprises VRDRSNGSGKRFESSNWFLDL (SEQ ID NO:441); and a light chain variable region, wherein CDR L1 comprises HNL, CDR L2 comprises DFN and CDR L3 comprises WAYEA (SEQ ID NO:408).

In some embodiments, the anti-HIV antibody comprises a heavy chain, wherein the heavy chain in the framework 3 region comprises an amino acid sequence selected from the group consisting of QLSQDPDDPDWG (SEQ ID NO:541) and LFSQDLYYPDRG (SEQ ID NO:542).

In some embodiments, the anti-HIV antibody is selected from the group consisting of:
a. an antibody comprising a heavy chain amino acid sequence comprising SEQ ID NO:1 or an antigen binding fragment thereof, except that the heavy chain amino acid sequence is modified whereby a part or all of the framework 3 region of SEQ ID NO:1 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:2 or an antigen binding fragment thereof;
b. an antibody comprising a heavy chain amino acid sequence comprising SEQ ID NO:3 or an antigen binding fragment thereof, except that the heavy chain amino acid sequence is modified whereby a part or all of the framework 3 region of SEQ ID NO:3 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:4 or an antigen binding fragment thereof;
c. an antibody comprising a heavy chain amino acid sequence comprising SEQ ID NO:5 or an antigen binding fragment thereof, except that the heavy chain amino acid sequence is modified whereby a part or all of the framework 3 region of SEQ ID NO:5 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:6 or an antigen binding fragment thereof;
d. an antibody comprising a heavy chain amino acid sequence comprising SEQ ID NO:7 or an antigen binding fragment thereof, except that the heavy chain amino acid sequence is modified whereby a part or all of the framework 3 region of SEQ ID NO:7 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:8 or an antigen binding fragment thereof;
e. an antibody comprising a heavy chain amino acid sequence comprising SEQ ID NO:9 or an antigen binding fragment thereof, except that the heavy chain amino acid sequence is modified whereby a part or all of the framework 3 region of SEQ ID NO:9 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:10 or an antigen binding fragment thereof;
f. an antibody comprising a heavy chain amino acid sequence comprising SEQ ID NO:11 or an antigen binding fragment thereof, except that the heavy chain amino acid sequence is modified whereby a part or all of the framework 3 region of SEQ ID NO:11 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:12 or an antigen binding fragment thereof;
g. an antibody comprising a heavy chain amino acid sequence comprising SEQ ID NO:13 or an antigen binding fragment thereof, except that the heavy chain amino acid sequence is modified whereby a part or all of the framework 3 region of SEQ ID NO:13 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:14 or an antigen binding fragment thereof;
h. an antibody comprising a heavy chain amino acid sequence comprising SEQ ID NO:15 or an antigen binding fragment thereof, except that the heavy chain amino acid sequence is modified whereby a part or all of the framework 3 region of SEQ ID NO:15 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:16 or an antigen binding fragment thereof;
i. an antibody comprising a heavy chain amino acid sequence comprising SEQ ID NO:17 or an antigen binding fragment thereof, except that the heavy chain amino acid sequence is modified whereby a part or all of the framework 3 region of SEQ ID NO:17 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:18 or an antigen binding fragment thereof;
j. an antibody comprising a heavy chain amino acid sequence comprising SEQ ID NO:19 or an antigen binding fragment thereof, except that the heavy chain amino acid sequence is modified whereby a part or all of the framework 3 region of SEQ ID NO:19 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:20 or an antigen binding fragment thereof;
k. an antibody comprising a heavy chain amino acid sequence comprising SEQ ID NO:21 or an antigen binding fragment thereof, except that the heavy chain amino acid sequence is modified whereby a part or all of the framework 3 region of SEQ ID NO:21 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:22 or an antigen binding fragment thereof;
l. an antibody comprising a heavy chain amino acid sequence comprising SEQ ID NO:23 or an antigen binding fragment thereof, except that the heavy chain amino acid sequence is modified whereby a part or all of the framework 3 region of SEQ ID NO:23 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:24 or an antigen binding fragment thereof;
m. an antibody comprising a heavy chain amino acid sequence comprising SEQ ID NO:25 or an antigen binding fragment thereof, except that the heavy chain amino acid sequence is modified whereby a part or all of the framework 3 region of SEQ ID NO:25 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:26 or an antigen binding fragment thereof;
n. an antibody comprising a heavy chain amino acid sequence comprising SEQ ID NO:27 or an antigen binding fragment thereof, except that the heavy chain amino acid sequence is modified whereby a part or all of the framework 3 region of SEQ ID NO:27 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:28 or an antigen binding fragment thereof;
o. an antibody comprising a heavy chain amino acid sequence comprising SEQ ID NO:29 or an antigen binding fragment thereof, except that the heavy chain amino acid sequence is modified whereby a part or all of the framework 3 region of SEQ ID NO:29 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:30 or an antigen binding fragment thereof;
p. an antibody comprising a heavy chain amino acid sequence comprising SEQ ID NO:31 or an antigen binding fragment thereof, except that the heavy chain amino acid sequence is modified whereby a part or all of the framework 3 region of SEQ ID NO:31 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:32 or an antigen binding fragment thereof;
q. an antibody comprising a heavy chain amino acid sequence comprising SEQ ID NO:33 or an antigen binding fragment thereof, except that the heavy chain amino acid sequence is modified whereby a part or all of the framework 3 region of SEQ ID NO:33 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:34 or an antigen binding fragment thereof;
r. an antibody comprising a heavy chain amino acid sequence comprising SEQ ID NO:35 or an antigen binding fragment thereof, except that the heavy chain amino acid sequence is modified whereby a part or all of the framework 3 region of SEQ ID NO:35 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:36 or an antigen binding fragment thereof;
s. an antibody comprising a heavy chain amino acid sequence comprising SEQ ID NO:37 or an antigen binding fragment thereof, except that the heavy chain amino acid sequence is modified whereby a part or all of the framework 3 region of SEQ ID NO:37 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:38 or an antigen binding fragment thereof;
t. an antibody comprising a heavy chain amino acid sequence comprising SEQ ID NO:39 or an antigen binding fragment thereof, except that the heavy chain amino acid sequence is modified whereby a part or all of the framework 3 region of SEQ ID NO:39 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:40 or an antigen binding fragment thereof;
u. an antibody comprising a heavy chain amino acid sequence comprising SEQ ID NO:41 or an antigen binding fragment thereof, except that the heavy chain amino acid sequence is modified whereby a part or all of the framework 3 region of SEQ ID NO:41 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:42 or an antigen binding fragment thereof;
v. an antibody comprising a heavy chain amino acid sequence comprising SEQ ID NO:43 or an antigen binding fragment thereof, except that the heavy chain amino acid sequence is modified whereby a part or all of the framework 3 region of SEQ ID NO:43 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:44 or an antigen binding fragment thereof;
w. an antibody comprising a heavy chain amino acid sequence comprising SEQ ID NO:45 or an antigen binding fragment thereof, except that the heavy chain amino acid sequence is modified whereby a part or all of the framework 3 region of SEQ ID NO:45 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:46 or an antigen binding fragment thereof;
x. an antibody comprising a heavy chain amino acid sequence comprising SEQ ID NO:47 or an antigen binding fragment thereof, except that the heavy chain amino acid sequence is modified whereby a part or all of the framework 3 region of SEQ ID NO:47 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:48 or an antigen binding fragment thereof;
y. an antibody comprising a heavy chain amino acid sequence comprising SEQ ID NO:49 or an antigen binding fragment thereof, except that the heavy chain amino acid sequence is modified whereby a part or all of the framework 3 region of SEQ ID NO:49 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:50 or an antigen binding fragment thereof;
z. an antibody comprising a heavy chain amino acid sequence comprising SEQ ID NO:51 or an antigen binding fragment thereof, except that the heavy chain amino acid sequence is modified whereby a part or all of the framework 3 region of SEQ ID NO:51 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:52 or an antigen binding fragment thereof;
aa. an antibody comprising a heavy chain amino acid sequence comprising SEQ ID NO:53 or an antigen binding fragment thereof, except that the heavy chain amino acid sequence is modified whereby a part or all of the framework 3 region of SEQ ID NO:53 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:54 or an antigen binding fragment thereof;
bb. an antibody comprising a heavy chain amino acid sequence comprising SEQ ID NO:55 or an antigen binding fragment thereof, except that the heavy chain amino acid sequence is modified whereby a part or all of the framework 3 region of SEQ ID NO:55 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:56 or an antigen binding fragment thereof;
cc. an antibody comprising a heavy chain amino acid sequence comprising SEQ ID NO:57 or an antigen binding fragment thereof, except that the heavy chain amino acid sequence is modified whereby a part or all of the framework 3 region of SEQ ID NO:57 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:58 or an antigen binding fragment thereof;
dd. an antibody comprising a heavy chain amino acid sequence comprising SEQ ID NO:59 or an antigen binding fragment thereof, except that the heavy chain amino acid sequence is modified whereby a part or all of the framework 3 region of SEQ ID NO:59 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:60 or an antigen binding fragment thereof;
ee. an antibody comprising a heavy chain amino acid sequence comprising SEQ ID NO:61 or an antigen binding fragment thereof, except that the heavy chain amino acid sequence is modified whereby a part or all of the framework 3 region of SEQ ID NO:61 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:62 or an antigen binding fragment thereof;
ff. an antibody comprising a heavy chain amino acid sequence comprising SEQ ID NO:63 or an antigen binding fragment thereof, except that the heavy chain amino acid sequence is modified whereby a part or all of the framework 3 region of SEQ ID NO:63 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:64 or an antigen binding fragment thereof;
gg. an antibody comprising a heavy chain amino acid sequence comprising SEQ ID NO:65 or an antigen binding fragment thereof, except that the heavy chain amino acid sequence is modified whereby a part or all of the framework 3 region of SEQ ID NO:65 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:66 or an antigen binding fragment thereof;
hh. an antibody comprising a heavy chain amino acid sequence comprising SEQ ID NO:67 or an antigen binding fragment thereof, except that the heavy chain amino acid sequence is modified whereby a part or all of the framework 3 region of SEQ ID NO:67 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:68 or an antigen binding fragment thereof;
ii. an antibody comprising a heavy chain amino acid sequence comprising SEQ ID NO:69 or an antigen binding fragment thereof, except that the heavy chain amino acid sequence is modified whereby a part or all of the framework 3 region of SEQ ID NO:69 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:70 or an antigen binding fragment thereof;
jj. an antibody comprising a heavy chain amino acid sequence comprising SEQ ID NO:71 or an antigen binding fragment thereof, except that the heavy chain amino acid sequence is modified whereby a part or all of the framework 3 region of SEQ ID NO:71 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:72 or an antigen binding fragment thereof;
kk. an antibody comprising a heavy chain amino acid sequence comprising SEQ ID NO:73 or an antigen binding fragment thereof, except that the heavy chain amino acid sequence is modified whereby a part or all of the framework 3 region of SEQ ID NO:73 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:74 or an antigen binding fragment thereof;
ll. an antibody comprising a heavy chain amino acid sequence comprising SEQ ID NO:75 or an antigen binding fragment thereof, except that the heavy chain amino acid sequence is modified whereby a part or all of the framework 3 region of SEQ ID NO:75 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:76 or an antigen binding fragment thereof;
mm. an antibody comprising a heavy chain amino acid sequence comprising SEQ ID NO:153 or an antigen binding fragment thereof, except that the heavy chain amino acid sequence is modified whereby a part or all of the framework 3 region of SEQ ID NO:153 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:155 or an antigen binding fragment thereof;
nn. an antibody comprising a heavy chain amino acid sequence comprising SEQ ID NO:157 or an antigen binding fragment thereof, except that the heavy chain amino acid sequence is modified whereby a part or all of the framework 3 region of SEQ ID NO:157 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:159 or an antigen binding fragment thereof;
oo. an antibody comprising a heavy chain amino acid sequence comprising SEQ ID NO:161 or an antigen binding fragment thereof, except that the heavy chain amino acid sequence is modified whereby a part or all of the framework 3 region of SEQ ID NO:161 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:163 or an antigen binding fragment thereof;
pp. an antibody comprising a heavy chain amino acid sequence comprising SEQ ID NO:165 or an antigen binding fragment thereof, except that the heavy chain amino acid sequence is modified whereby a part or all of the framework 3 region of SEQ ID NO:165 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:167 or an antigen binding fragment thereof;
qq. an antibody comprising a heavy chain amino acid sequence comprising SEQ ID NO:169 or an antigen binding fragment thereof, except that the heavy chain amino acid sequence is modified whereby a part or all of the framework 3 region of SEQ ID NO:169 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:171 or an antigen binding fragment thereof;
rr. an antibody comprising a heavy chain amino acid sequence comprising SEQ ID NO:173 or an antigen binding fragment thereof, except that the heavy chain amino acid sequence is modified whereby a part or all of the framework 3 region of SEQ ID NO:173 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:175 or an antigen binding fragment thereof;
ss. an antibody comprising a heavy chain amino acid sequence comprising SEQ ID NO:177 or an antigen binding fragment thereof, except that the heavy chain amino acid sequence is modified whereby a part or all of the framework 3 region of SEQ ID NO:177 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:179 or an antigen binding fragment thereof;
tt. an antibody comprising a heavy chain amino acid sequence comprising SEQ ID NO:181 or an antigen binding fragment thereof, except that the heavy chain amino acid sequence is modified whereby a part or all of the framework 3 region of SEQ ID NO:181 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:183 or an antigen binding fragment thereof;
uu. an antibody comprising a heavy chain amino acid sequence comprising SEQ ID NO:185 or an antigen binding fragment thereof, except that the heavy chain amino acid sequence is modified whereby a part or all of the framework 3 region of SEQ ID NO:185 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:187 or an antigen binding fragment thereof;
vv. an antibody comprising a heavy chain amino acid sequence comprising SEQ ID NO:189 or an antigen binding fragment thereof, except that the heavy chain amino acid sequence is modified whereby a part or all of the framework 3 region of SEQ ID NO:189 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:191 or an antigen binding fragment thereof;
ww. an antibody comprising a heavy chain amino acid sequence comprising SEQ ID NO:193 or an antigen binding fragment thereof, except that the heavy chain amino acid sequence is modified whereby a part or all of the framework 3 region of SEQ ID NO:193 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:195 or an antigen binding fragment thereof;
xx. an antibody comprising a heavy chain amino acid sequence comprising SEQ ID NO:197 or an antigen binding fragment thereof, except that the heavy chain amino acid sequence is modified whereby a part or all of the framework 3 region of SEQ ID NO:197 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:199 or an antigen binding fragment thereof;
yy. an antibody comprising a heavy chain amino acid sequence comprising SEQ ID NO:201 or an antigen binding fragment thereof, except that the heavy chain amino acid sequence is modified whereby a part or all of the framework 3 region of SEQ ID NO:201 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:203 or an antigen binding fragment thereof;
zz. an antibody comprising a heavy chain amino acid sequence comprising SEQ ID NO:205 or an antigen binding fragment thereof, except that the heavy chain amino acid sequence is modified whereby a part or all of the framework 3 region of SEQ ID NO:205 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:207 or an antigen binding fragment thereof;
aaa. an antibody comprising a heavy chain amino acid sequence comprising SEQ ID NO:209 or an antigen binding fragment thereof, except that the heavy chain amino acid sequence is modified whereby a part or all of the framework 3 region of SEQ ID NO:209 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:211 or an antigen binding fragment thereof;
bbb. an antibody comprising a heavy chain amino acid sequence comprising SEQ ID NO:213 or an antigen binding fragment thereof, except that the heavy chain amino acid sequence is modified whereby a part or all of the framework 3 region of SEQ ID NO:213 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:215 or an antigen binding fragment thereof;
ccc. an antibody comprising a heavy chain amino acid sequence comprising SEQ ID NO:217 or an antigen binding fragment thereof, except that the heavy chain amino acid sequence is modified whereby a part or all of the framework 3 region of SEQ ID NO:217 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:219 or an antigen binding fragment thereof;
ddd. an antibody comprising a heavy chain amino acid sequence comprising SEQ ID NO:221 or an antigen binding fragment thereof, except that the heavy chain amino acid sequence is modified whereby a part or all of the framework 3 region of SEQ ID NO:221 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:223 or an antigen binding fragment thereof;
eee. an antibody comprising a heavy chain amino acid sequence comprising SEQ ID NO:225 or an antigen binding fragment thereof, except that the heavy chain amino acid sequence is modified whereby a part or all of the framework 3 region of SEQ ID NO:225 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:227 or an antigen binding fragment thereof;
fff. an antibody comprising a heavy chain amino acid sequence comprising SEQ ID NO:229 or an antigen binding fragment thereof, except that the heavy chain amino acid sequence is modified whereby a part or all of the framework 3 region of SEQ ID NO:229 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:231 or an antigen binding fragment thereof;
ggg. an antibody comprising a heavy chain amino acid sequence comprising SEQ ID NO:233 or an antigen binding fragment thereof, except that the heavy chain amino acid sequence is modified whereby a part or all of the framework 3 region of SEQ ID NO:233 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:235 or an antigen binding fragment thereof;
hhh. an antibody comprising a heavy chain amino acid sequence comprising SEQ ID NO:237 or an antigen binding fragment thereof, except that the heavy chain amino acid sequence is modified whereby a part or all of the framework 3 region of SEQ ID NO:237 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:239 or an antigen binding fragment thereof;
iii. an antibody comprising a heavy chain amino acid sequence comprising SEQ ID NO:241 or an antigen binding fragment thereof, except that the heavy chain amino acid sequence is modified whereby a part or all of the framework 3 region of SEQ ID NO:241 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:243 or an antigen binding fragment thereof;
jjj. an antibody comprising a heavy chain amino acid sequence comprising SEQ ID NO:245 or an antigen binding fragment thereof, except that the heavy chain amino acid sequence is modified whereby a part or all of the framework 3 region of SEQ ID NO:245 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:247 or an antigen binding fragment thereof;
kkk. an antibody comprising a heavy chain amino acid sequence comprising SEQ ID NO:249 or an antigen binding fragment thereof, except that the heavy chain amino acid sequence is modified whereby a part or all of the framework 3 region of SEQ ID NO:249 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:251 or an antigen binding fragment thereof;
lll. an antibody comprising a heavy chain amino acid sequence comprising SEQ ID NO:253 or an antigen binding fragment thereof, except that the heavy chain amino acid sequence is modified whereby a part or all of the framework 3 region of SEQ ID NO:253 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:255 or an antigen binding fragment thereof;
mmm. an antibody comprising a heavy chain amino acid sequence comprising SEQ ID NO:257 or an antigen binding fragment thereof, except that the heavy chain amino acid sequence is modified whereby a part or all of the framework 3 region of SEQ ID NO:257 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:259 or an antigen binding fragment thereof;
nnn. an antibody comprising a heavy chain amino acid sequence comprising SEQ ID NO:261 or an antigen binding fragment thereof, except that the heavy chain amino acid sequence is modified whereby a part or all of the framework 3 region of SEQ ID NO:261 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:263 or an antigen binding fragment thereof;
ooo. an antibody comprising a heavy chain amino acid sequence comprising SEQ ID NO:265 or an antigen binding fragment thereof, except that the heavy chain amino acid sequence is modified whereby a part or all of the framework 3 region of SEQ ID NO:265 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:267 or an antigen binding fragment thereof;
ppp. an antibody comprising a heavy chain amino acid sequence comprising SEQ ID NO:269 or an antigen binding fragment thereof, except that the heavy chain amino acid sequence is modified whereby a part or all of the framework 3 region of SEQ ID NO:269 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:271 or an antigen binding fragment thereof;
qqq. an antibody comprising a heavy chain amino acid sequence comprising SEQ ID NO:273 or an antigen binding fragment thereof, except that the heavy chain amino acid sequence is modified whereby a part or all of the framework 3 region of SEQ ID NO:273 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:275 or an antigen binding fragment thereof;
rrr. an antibody comprising a heavy chain amino acid sequence comprising SEQ ID NO:277 or an antigen binding fragment thereof, except that the heavy chain amino acid sequence is modified whereby a part or all of the framework 3 region of SEQ ID NO:277 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:279 or an antigen binding fragment thereof;
sss. an antibody comprising a heavy chain amino acid sequence comprising SEQ ID NO:281 or an antigen binding fragment thereof, except that the heavy chain amino acid sequence is modified whereby a part or all of the framework 3 region of SEQ ID NO:281 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:283 or an antigen binding fragment thereof;
ttt. an antibody comprising a heavy chain amino acid sequence comprising SEQ ID NO:285 or an antigen binding fragment thereof, except that the heavy chain amino acid sequence is modified whereby a part or all of the framework 3 region of SEQ ID NO:285 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:287 or an antigen binding fragment thereof;
uuu. an antibody comprising a heavy chain amino acid sequence comprising SEQ ID NO:289 or an antigen binding fragment thereof, except that the heavy chain amino acid sequence is modified whereby a part or all of the framework 3 region of SEQ ID NO:289 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:291 or an antigen binding fragment thereof;
vvv. an antibody comprising a heavy chain amino acid sequence comprising SEQ ID NO:293 or an antigen binding fragment thereof, except that the heavy chain amino acid sequence is modified whereby a part or all of the framework 3 region of SEQ ID NO:293 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:295 or an antigen binding fragment thereof;
www. an antibody comprising a heavy chain amino acid sequence comprising SEQ ID NO:297 or an antigen binding fragment thereof, except that the heavy chain amino acid sequence is modified whereby a part or all of the framework 3 region of SEQ ID NO:297 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:299 or an antigen binding fragment thereof;
xxx. an antibody comprising a heavy chain amino acid sequence comprising SEQ ID NO:301 or an antigen binding fragment thereof, except that the heavy chain amino acid sequence is modified whereby a part or all of the framework 3 region of SEQ ID NO:301 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:303 or an antigen binding fragment thereof;
yyy. an antibody comprising a heavy chain amino acid sequence comprising SEQ ID NO:305 or an antigen binding fragment thereof, except that the heavy chain amino acid sequence is modified whereby a part or all of the framework 3 region of SEQ ID NO:305 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:307 or an antigen binding fragment thereof;
zzz. an antibody comprising a heavy chain amino acid sequence comprising SEQ ID NO:309 or an antigen binding fragment thereof, except that the heavy chain amino acid sequence is modified whereby a part or all of the framework 3 region of SEQ ID NO:309 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:311 or an antigen binding fragment thereof;
aaaa. an antibody comprising a heavy chain amino acid sequence comprising SEQ ID NO:313 or an antigen binding fragment thereof, except that the heavy chain amino acid sequence is modified whereby a part or all of the framework 3 region of SEQ ID NO:313 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:315 or an antigen binding fragment thereof;
bbbb. an antibody comprising a heavy chain amino acid sequence comprising SEQ ID NO:317 or an antigen binding fragment thereof, except that the heavy chain amino acid sequence is modified whereby a part or all of the framework 3 region of SEQ ID NO:317 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:319 or an antigen binding fragment thereof;
cccc. an antibody comprising a heavy chain amino acid sequence comprising SEQ ID NO:321 or an antigen binding fragment thereof, except that the heavy chain amino acid sequence is modified whereby a part or all of the framework 3 region of SEQ ID NO:321 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:323 or an antigen binding fragment thereof;
dddd. an antibody comprising a heavy chain amino acid sequence comprising SEQ ID NO:325 or an antigen binding fragment thereof, except that the heavy chain amino acid sequence is modified whereby a part or all of the framework 3 region of SEQ ID NO:325 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:327 or an antigen binding fragment thereof;
eeee. an antibody comprising a heavy chain amino acid sequence comprising SEQ ID NO:329 or an antigen binding fragment thereof, except that the heavy chain amino acid sequence is modified whereby a part or all of the framework 3 region of SEQ ID NO:329 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:331 or an antigen binding fragment thereof;
ffff. an antibody comprising a heavy chain amino acid sequence comprising SEQ ID NO:333 or an antigen binding fragment thereof, except that the heavy chain amino acid sequence is modified whereby a part or all of the framework 3 region of SEQ ID NO:333 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:335 or an antigen binding fragment thereof;
gggg. an antibody comprising a heavy chain amino acid sequence comprising SEQ ID NO:337 or an antigen binding fragment thereof, except that the heavy chain amino acid sequence is modified whereby a part or all of the framework 3 region of SEQ ID NO:337 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:339 or an antigen binding fragment thereof;
hhhh. an antibody comprising a heavy chain amino acid sequence comprising SEQ ID NO:341 or an antigen binding fragment thereof, except that the heavy chain amino acid sequence is modified whereby a part or all of the framework 3 region of SEQ ID NO:341 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:343 or an antigen binding fragment thereof;
iiii. an antibody comprising a heavy chain amino acid sequence comprising SEQ ID NO:345 or an antigen binding fragment thereof, except that the heavy chain amino acid sequence is modified whereby a part or all of the framework 3 region of SEQ ID NO:345 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:347 or an antigen binding fragment thereof;
jjjj. an antibody comprising a heavy chain amino acid sequence comprising SEQ ID NO:349 or an antigen binding fragment thereof, except that the heavy chain amino acid sequence is modified whereby a part or all of the framework 3 region of SEQ ID NO:349 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:351 or an antigen binding fragment thereof;
kkkk. an antibody comprising a heavy chain amino acid sequence comprising SEQ ID NO:353 or an antigen binding fragment thereof, except that the heavy chain amino acid sequence is modified whereby a part or all of the framework 3 region of SEQ ID NO:353 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:355 or an antigen binding fragment thereof;
llll. an antibody comprising a heavy chain amino acid sequence comprising SEQ ID NO:357 or an antigen binding fragment thereof, except that the heavy chain amino acid sequence is modified whereby a part or all of the framework 3 region of SEQ ID NO:357 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:359 or an antigen binding fragment thereof;
mmmm. an antibody comprising a heavy chain amino acid sequence comprising SEQ ID NO:361 or an antigen binding fragment thereof, except that the heavy chain amino acid sequence is modified whereby a part or all of the framework 3 region of SEQ ID NO:361 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:363 or an antigen binding fragment thereof;
nnnn. an antibody comprising a heavy chain amino acid sequence comprising SEQ ID NO:365 or an antigen binding fragment thereof, except that the heavy chain amino acid sequence is modified whereby a part or all of the framework 3 region of SEQ ID NO:365 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:367 or an antigen binding fragment thereof;
oooo. an antibody comprising a heavy chain amino acid sequence comprising SEQ ID NO:369 or an antigen binding fragment thereof, except that the heavy chain amino acid sequence is modified whereby a part or all of the framework 3 region of SEQ ID NO:369 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:371 or an antigen binding fragment thereof;
pppp. an antibody comprising a heavy chain amino acid sequence comprising SEQ ID NO:373 or an antigen binding fragment thereof, except that the heavy chain amino acid sequence is modified whereby a part or all of the framework 3 region of SEQ ID NO:373 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:375 or an antigen binding fragment thereof;
qqqq. an antibody comprising a heavy chain amino acid sequence comprising SEQ ID NO:377 or an antigen binding fragment thereof, except that the heavy chain amino acid sequence is modified whereby a part or all of the framework 3 region of SEQ ID NO:377 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:379 or an antigen binding fragment thereof;
rrrr. an antibody comprising a heavy chain amino acid sequence comprising SEQ ID NO:381 or an antigen binding fragment thereof, except that the heavy chain amino acid sequence is modified whereby a part or all of the framework 3 region of SEQ ID NO:381 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:383 or an antigen binding fragment thereof;
ssss. an antibody comprising a heavy chain amino acid sequence comprising SEQ ID NO:385 or an antigen binding fragment thereof, except that the heavy chain amino acid sequence is modified whereby a part or all of the framework 3 region of SEQ ID NO:385 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:387 or an antigen binding fragment thereof;
tttt. an antibody comprising a heavy chain amino acid sequence comprising SEQ ID NO:389 or an antigen binding fragment thereof, except that the heavy chain amino acid sequence is modified whereby a part or all of the framework 3 region of SEQ ID NO:389 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:391 or an antigen binding fragment thereof;
uuuu. an antibody comprising a heavy chain amino acid sequence comprising SEQ ID NO:393 or an antigen binding fragment thereof, except that the heavy chain amino acid sequence is modified whereby a part or all of the framework 3 region of SEQ ID NO:393 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:395 or an antigen binding fragment thereof; and
vvvv. an antibody comprising a heavy chain amino acid sequence comprising SEQ ID NO:397 or an antigen binding fragment thereof, except that the heavy chain amino acid sequence is modified whereby a part or all of the framework 3 region of SEQ ID NO:397 is replaced with an amino acid sequence selected from the group consisting of QLSQDPDDPDWG (SEQ ID NO:541) and LFSQDLYYPDRG (SEQ ID NO:542), and a light chain amino acid sequence comprising SEQ ID NO:399 or an antigen binding fragment thereof.

In some embodiments, the anti-HIV antibody comprises a heavy chain or an antigen binding fragment thereof and a light chain or an antigen binding fragment thereof, wherein the heavy chain comprises a heavy chain variable (VH) region and the light chain comprises a light chain variable (VL) region; wherein the VL region comprises one or more VL complementary determining regions (CDRs) and wherein the VH region comprises one or more VH complementary determining regions (CDRs), wherein the VL CDRs correspond to the CDRs found within any of SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 155, 159, 163, 167, 171, 175, 179, 183, 187, 191, 195, 199, 203, 207, 211, 215, 219, 223, 227, 231, 235, 239, 243, 247, 251,255, 259, 263, 267, 271, 275, 279, 283, 287, 291,295, 299, 303, 307, 311, 315, 319, 323, 327, 331, 335, 339, 343, 347, 351, 355, 359, 363, 367, 371, 375, 379, 383, 387, 391, 395, 399, 503, 511, 515, 519, 523, 527, 531, 535, and 539. In some embodiments, the heavy chain in the framework 3 region comprises an amino acid sequence selected from the group consisting of QLSQDPDDPDWG (SEQ ID NO:541) and LFSQDLYYPDRG (SEQ ID NO:542).

In some embodiments, the anti-HIV antibody comprises a heavy chain or an antigen binding fragment thereof and a light chain or an antigen binding fragment thereof, wherein the heavy chain comprises a heavy chain variable (VH) region and the light chain comprises a light chain variable (VL) region; wherein the VL region comprises one or more VL complementary determining regions (CDRs) and wherein the VH region comprises one or more VH complementary determining regions (CDRs), wherein the VH CDRs correspond to the CDRs found within any of SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 153, 157, 161, 165, 169, 173, 177, 181, 185, 189, 193, 197, 201, 205, 209, 213,217, 221, 225, 229, 233, 237, 241, 245, 249, 253, 257, 261, 265, 269, 273, 277, 281, 285, 289, 293, 297, 301, 305, 309, 313, 317, 321, 325, 329, 333, 337, 341, 345, 349, 353, 357, 361, 365, 369, 373, 377, 381, 385, 389, 393, 397, 501, 505, 507, 509, 513, 517, 521, 525, 529, 533, and 537. In some embodiments, the heavy chain in the framework 3 region comprises an amino acid sequence selected from the group consisting of QLSQDPDDPDWG (SEQ ID NO:541) and LFSQDLYYPDRG (SEQ ID NO:542).

In some embodiments, the anti-HIV antibody comprises a heavy chain or an antigen binding fragment thereof and a light chain or an antigen binding fragment thereof, wherein the heavy chain comprises a heavy chain variable (VH) region and the light chain comprises a light chain variable (VL) region; wherein the VL region comprises one or more VL complementary determining regions (CDRs) and wherein the VH region comprises one or more VH complementary determining regions (CDRs), wherein the VL CDRs correspond to the CDRs found within any of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 155, 159, 163, 167, 171, 175, 179, 183, 187, 191, 195, 199, 203, 207, 211, 215, 219, 223, 227, 231, 235, 239, 243, 247, 251,255, 259, 263, 267, 271, 275, 279, 283, 287, 291,295, 299, 303, 307, 311, 315, 319, 323, 327, 331, 335, 339, 343, 347, 351, 355, 359, 363, 367, 371, 375, 379, 383, 387, 391, 395, 399, 503, 511, 515, 519, 523, 527, 531, 535, and 539, or a variant thereof comprising 1, 2, 3, or 4 conservative amino acid substitutions, and wherein the VH CDRs correspond to the CDRs found within any of SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 153, 157, 161, 165, 169, 173, 177, 181, 185, 189, 193, 197, 201, 205, 209, 213, 217, 221, 225, 229, 233, 237, 241, 245, 249, 253, 257, 261, 265, 269, 273, 277, 281, 285, 289, 293, 297, 301, 305, 309, 313, 317, 321, 325, 329, 333, 337, 341, 345, 349, 353, 357, 361, 365, 369, 373, 377, 381, 385, 389, 393, 397, 501, 505, 507, 509, 513, 517, 521, 525, 529, 533, and 537 or a variant thereof comprising 1, 2, 3, or 4 conservative amino acid substitutions. In some embodiments, the heavy chain in the framework 3 region comprises an amino acid sequence selected from the group consisting of QLSQDPDDPDWG (SEQ ID NO:541) and LFSQDLYYPDRG (SEQ ID NO:542).

In some embodiments, the anti-HIV antibody comprises a heavy chain or an antigen binding fragment thereof and a light chain or an antigen binding fragment thereof, wherein the heavy chain comprises a heavy chain variable (VH) region and the light chain comprises a light chain variable (VL) region; wherein the VL region comprises an amino acid sequence selected from the group consisting of: amino acids 1-99 of SEQ ID NO:2; amino acids 1-97 of SEQ ID NO:4; amino acids 1-99 of SEQ ID NO:6; amino acids 1-99 of SEQ ID NO:8; amino acids 1-99 of SEQ ID NO:10; amino acids 1-99 of SEQ ID NO:12; amino acids 1-99 of SEQ ID NO:14; amino acids 1-99 of SEQ ID NO:16; amino acids 1-99 of SEQ ID NO:18; amino acids 1-99 of SEQ ID NO:20; amino acids 1-99 of SEQ ID NO:22; amino acids 1-99 of SEQ ID NO:24; amino acids 1-99 of SEQ ID NO:26; amino acids 1-99 of SEQ ID NO:28; amino acids 1-99 of SEQ ID NO:30; amino acids 1-99 of SEQ ID NO:32; amino acids 1-99 of SEQ ID NO:34; amino acids 1-100 of SEQ ID NO:36; amino acids 1-97 of SEQ ID NO:38; amino acids 1-100 of SEQ ID NO:40; amino acids 1-97 of SEQ ID NO:42; amino acids 1-97 of SEQ ID NO:44; amino acids 1-101 of SEQ ID NO:46; amino acids 1-101 of SEQ ID NO:48; amino acids 1-96 of SEQ ID NO:50; amino acids 1-97 of SEQ ID NO:52; amino acids 1-99 of SEQ ID NO:54; amino acids 1-99 of SEQ ID NO:56; amino acids 1-99 of SEQ ID NO:58; amino acids 1-99 of SEQ ID NO:60; amino acids 1-98 of SEQ ID NO:62; amino acids 1-99 of SEQ ID NO:64; amino acids 1-99 of SEQ ID NO:66; amino acids 1-96 of SEQ ID NO:68; amino acids 1-96 of SEQ ID NO:70; amino acids 1-96 of SEQ ID NO:72; amino acids 1-101 of SEQ ID NO:74; amino acids 1-97 of SEQ ID NO:76; amino acids 1-99 of SEQ ID NO:155; amino acids 1-99 of SEQ ID NO:159; amino acids 1-97 of SEQ ID NO:163; amino acids 1-97 of SEQ ID NO:167; amino acids 1-97 of SEQ ID NO:171; amino acids 1-97 of SEQ ID NO:175; amino acids 1-97 of SEQ ID NO:179; amino acids 1-97 of SEQ ID NO:183; amino acids 1-97 of SEQ ID NO:187; amino acids 1-97 of SEQ ID NO:191; amino acids 1-97 of SEQ ID NO:195; amino acids 1-97 of SEQ ID NO:199; amino acids 1-99 of SEQ ID NO:203; amino acids 1-99 of SEQ ID NO:207; amino acids 1-100 of SEQ ID NO:211; amino acids 1-99 of SEQ ID NO:215; amino acids 1-97 of SEQ ID NO:219; amino acids 1-99 of SEQ ID NO:223; amino acids 1-99 of SEQ ID NO:227; amino acids 1-99 of SEQ ID NO:231; amino acids 1-99 of SEQ ID NO:235; amino acids 1-99 of SEQ ID NO:239; amino acids 1-99 of SEQ ID NO:243; amino acids 1-99 of SEQ ID NO:247; amino acids 1-99 of SEQ ID NO:251; amino acids 1-99 of SEQ ID NO:255; amino acids 1-99 of SEQ ID NO:259; amino acids 1-99 of SEQ ID NO:263; amino acids 1-99 of SEQ ID NO:267; amino acids 1-99 of SEQ ID NO:271; amino acids 1-99 of SEQ ID NO:275; amino acids 1-99 of SEQ ID NO:279; amino acids 1-99 of SEQ ID NO:283; amino acids 1-99 of SEQ ID NO:287; amino acids 1-99 of SEQ ID NO:291; amino acids 1-100 of SEQ ID NO:295; amino acids 1-100 of SEQ ID NO:299; amino acids 1-100 of SEQ ID NO:303; amino acids 1-100 of SEQ ID NO:307; amino acids 1-100 of SEQ ID NO:311; amino acids 1-100 of SEQ ID NO:315; amino acids 1-97 of SEQ ID NO:319; amino acids 1-100 of SEQ ID NO:323; amino acids 1-97 of SEQ ID NO:327; amino acids 1-97 of SEQ ID NO:331; amino acids 1-97 of SEQ ID NO:335; amino acids 1-101 of SEQ ID NO:339; amino acids 1-101 of SEQ ID NO:343; amino acids 1-96 of SEQ ID NO:347; amino acids 1-97 of SEQ ID NO:351; amino acids 1-99 of SEQ ID NO:355; amino acids 1-99 of SEQ ID NO:359; amino acids 1-99 of SEQ ID NO:363; amino acids 1-99 of SEQ ID NO:367; amino acids 1-98 of SEQ ID NO:371; amino acids 1-99 of SEQ ID NO:375; amino acids 1-99 of SEQ ID NO:379; amino acids 1-96 of SEQ ID NO:383; amino acids 1-96 of SEQ ID NO:387; amino acids 1-96 of SEQ ID NO:391; amino acids 1-101 of SEQ ID NO:395; amino acids 1-97 of SEQ ID NO:399; amino acids 1-97 of SEQ ID NO:503; amino acids 1-97 of SEQ ID NO: 511; amino acids 1-97 of SEQ ID NO:515; amino acids 1-97 of SEQ ID NO:519; amino acids 1-97 of SEQ ID NO:523; amino acids 1-97 of SEQ ID NO:527; amino acids 1-97 of SEQ ID NO:531; amino acids 1-97 of SEQ ID NO:535; and amino acids 1-97 of SEQ ID NO:539, or a variant thereof comprising 1, 2, 3, or 4 conservative amino acid substitutions. In some embodiments, the heavy chain in the framework 3 region comprises an amino acid sequence selected from the group consisting of QLSQDPDDPDWG (SEQ ID NO:541) and LFSQDLYYPDRG (SEQ ID NO:542).

In some embodiments, the anti-HIV antibody comprises a heavy chain or an antigen binding fragment thereof and a light chain or an antigen binding fragment thereof, wherein the heavy chain comprises a heavy chain variable (VH) region and the light chain comprises a light chain variable (VL) region; wherein the VH region comprises an amino acid sequence selected from the group consisting of: amino acids 1-128 of SEQ ID NO:1; amino acids 1-127 of SEQ ID NO:3; amino acids 1-127 of SEQ ID NO:5; amino acids 1-128 of SEQ ID NO:7; amino acids 1-127 of SEQ ID NO:9; amino acids 1-127 of SEQ ID NO:11; amino acids 1-127 of SEQ ID NO:13; amino acids 1-127 of SEQ ID NO:15; amino acids 1-127 of SEQ ID NO:17; amino acids 1-127 of SEQ ID NO:19; amino acids 1-127 of SEQ ID NO:21; amino acids 1-127 of SEQ ID NO:23; amino acids 1-127 of SEQ ID NO:25; amino acids 1-127 of SEQ ID NO:27; amino acids 1-127 of SEQ ID NO:29; amino acids 1-127 of SEQ ID NO:31; amino acids 1-127 of SEQ ID NO:33; amino acids 1-120 of SEQ ID NO:35; amino acids 1-120 of SEQ ID NO:37; amino acids 1-123 of SEQ ID NO:39; amino acids 1-120 of SEQ ID NO:41; amino acids 1-120 of SEQ ID NO:43; amino acids 1-125 of SEQ ID NO:45; amino acids 1-125 of SEQ ID NO:47; amino acids 1-120 of SEQ ID NO:49; amino acids 1-120 of SEQ ID NO:51; amino acids 1-121 of SEQ ID NO:53; amino acids 1-121 of SEQ ID NO:55; amino acids 1-121 of SEQ ID NO:57; amino acids 1-121 of SEQ ID NO:59; amino acids 1-120 of SEQ ID NO:61; amino acids 1-121 of SEQ ID NO:63; amino acids 1-121 of SEQ ID NO:65; amino acids 1-120 of SEQ ID NO:67; amino acids 1-120 of SEQ ID NO:69; amino acids 1-120 of SEQ ID NO:71; amino acids 1-125 of SEQ ID NO:73; amino acids 1-120 of SEQ ID NO:75; amino acids 1-128 of SEQ ID NO:153; amino acids 1-128 of SEQ ID NO:157; amino acids 1-127 of SEQ ID NO:161; amino acids 1-127 of SEQ ID NO:165; amino acids 1-127 of SEQ ID NO:169; amino acids 1-127 of SEQ ID NO:173; amino acids 1-127 of SEQ ID NO:177; amino acids 1-127 of SEQ ID NO:181; amino acids 1-127 of SEQ ID NO:185; amino acids 1-127 of SEQ ID NO:189; amino acids 1-127 of SEQ ID NO:193; amino acids 1-127 of SEQ ID NO:197; amino acids 1-127 of SEQ ID NO:201; amino acids 1-127 of SEQ ID NO:205; amino acids 1-127 of SEQ ID NO:209; amino acids 1-127 of SEQ ID NO:213; amino acids 1-127 of SEQ ID NO:217; amino acids 1-127 of SEQ ID NO:221; amino acids 1-128 of SEQ ID NO:225; amino acids 1-127 of SEQ ID NO:229; amino acids 1-127 of SEQ ID NO:233; amino acids 1-127 of SEQ ID NO:237; amino acids 1-127 of SEQ ID NO:241; amino acids 1-127 of SEQ ID NO:245; amino acids 1-127 of SEQ ID NO:249; amino acids 1-127 of SEQ ID NO:253; amino acids 1-127 of SEQ ID NO:257; amino acids 1-127 of SEQ ID NO:261; amino acids 1-127 of SEQ ID NO:265; amino acids 1-127 of SEQ ID NO:269; amino acids 1-127 of SEQ ID NO:273; amino acids 1-127 of SEQ ID NO:277; amino acids 1-127 of SEQ ID NO:281; amino acids 1-127 of SEQ ID NO:285; amino acids 1-127 of SEQ ID NO:289; amino acids 1-120 of SEQ ID NO:293; amino acids 1-120 of SEQ ID NO:297; amino acids 1-120 of SEQ ID NO:301; amino acids 1-123 of SEQ ID NO:305; amino acids 1-128 of SEQ ID NO:309; amino acids 1-128 of SEQ ID NO:313; amino acids 1-120 of SEQ ID NO:317; amino acids 1-123 of SEQ ID NO:321; amino acids 1-120 of SEQ ID NO:325; amino acids 1-120 of SEQ ID NO:329; amino acids 1-120 of SEQ ID NO:333; amino acids 1-125 of SEQ ID NO:337; amino acids 1-125 of SEQ ID NO:341; amino acids 1-120 of SEQ ID NO:345; amino acids 1-120 of SEQ ID NO:349; amino acids 1-121 of SEQ ID NO:353; amino acids 1-121 of SEQ ID NO:357; amino acids 1-121 of SEQ ID NO:361; amino acids 1-121 of SEQ ID NO:365; amino acids 1-120 of SEQ ID NO:369; amino acids 1-121 of SEQ ID 30 NO:373; amino acids 1-121 of SEQ ID NO:377; amino acids 1-120 of SEQ ID NO:381; amino acids 1-120 of SEQ ID NO:385; amino acids 1-120 of SEQ ID NO:389; amino acids 1-125 of SEQ ID NO:393; and amino acids 1-120 of SEQ ID NO:397, or a variant thereof comprising 1, 2, 3, or 4 conservative amino acid substitutions. In some embodiments, the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542).

In some embodiments, the anti-HIV antibody comprises a heavy chain or an antigen binding fragment thereof and a light chain or an antigen binding fragment thereof, wherein the heavy chain comprises a heavy chain variable (VH) region and the light chain comprises a light chain variable (VL) region; wherein the VH region comprises an amino acid sequence selected from the group consisting of: amino acids 1-134 of SEQ ID NO:501; amino acids 1-127 of SEQ ID NO: 505; amino acids 1-127 of SEQ ID NO:507; amino acids 1-127 of SEQ ID NO:509; amino acids 1-127 of SEQ ID NO:513; amino acids 1-127 of SEQ ID NO:517; amino acids 1-127 of SEQ ID NO:521; amino acids 1-127 of SEQ ID NO:525; amino acids 1-127 of SEQ ID NO:529; amino acids 1-127 of SEQ ID NO:533; and amino acids 1-127 of SEQ ID NO:537, or a variant thereof comprising 1, 2, 3, or 4 conservative amino acid substitutions.

In some embodiments, the anti-HIV antibody comprises a heavy chain or an antigen binding fragment thereof and a light chain or an antigen binding fragment thereof, wherein the heavy chain or antigen binding fragment thereof comprises a heavy chain variable (VH) region and the light chain or antigen binding fragment thereof comprises a light chain variable (VL) region; wherein the anti-HIV antibody is selected from the group consisting of:
a) an antibody wherein the VH region comprises amino acids 1-128 of SEQ ID NO:1 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:2 or a variant thereof comprising 1, 2, 3, or 4 conservative amino acid substitutions;
b) an antibody wherein the VH region comprises amino acids 1-127 of SEQ ID NO:3 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-97 of SEQ ID NO:4 or a variant thereof comprising 1, 2, 3, or 4 conservative amino acid substitutions;
c) an antibody wherein the VH region comprises amino acids 1-127 of SEQ ID NO:5 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:6 or a variant thereof comprising 1, 2, 3, or 4 conservative amino acid substitutions;
d) an antibody wherein the VH region comprises amino acids 1-128 of SEQ ID NO:7 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:8 or a variant thereof comprising 1, 2, 3, or 4 conservative amino acid substitutions;
e) an antibody wherein the VH region comprises amino acids 1-127 of SEQ ID NO:9 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:10 or a variant thereof comprising 1, 2, 3, or 4 conservative amino acid substitutions;
f) an antibody wherein the VH region comprises amino acids 1-127 of SEQ ID NO:11 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:12 or a variant thereof comprising 1, 2, 3, or 4 conservative amino acid substitutions;
g) an antibody wherein the VH region comprises amino acids 1-127 of SEQ ID NO:13 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:14 or a variant thereof comprising 1, 2, 3, or 4 conservative amino acid substitutions;
h) an antibody wherein the VH region comprises amino acids 1-127 of SEQ ID NO:15 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:16 or a variant thereof comprising 1, 2, 3, or 4 conservative amino acid substitutions;
i) an antibody wherein the VH region comprises amino acids 1-127 of SEQ ID NO:17 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:18 or a variant thereof comprising 1, 2, 3, or 4 conservative amino acid substitutions;
j) an antibody wherein the VH region comprises amino acids 1-127 of SEQ ID NO:19 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:20 or a variant thereof comprising 1, 2, 3, or 4 conservative amino acid substitutions;
k) an antibody wherein the VH region comprises amino acids 1-127 of SEQ ID NO:21 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:22 or a variant thereof comprising 1, 2, 3, or 4 conservative amino acid substitutions;
l) an antibody wherein the VH region comprises amino acids 1-127 of SEQ ID NO:23 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:24 or a variant thereof comprising 1, 2, 3, or 4 conservative amino acid substitutions;
m) an antibody wherein the VH region comprises amino acids 1-127 of SEQ ID NO:25 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:26 or a variant thereof comprising 1, 2, 3, or 4 conservative amino acid substitutions;
n) an antibody wherein the VH region comprises amino acids 1-127 of SEQ ID NO:27 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:28 or a variant thereof comprising 1, 2, 3, or 4 conservative amino acid substitutions;
o) an antibody wherein the VH region comprises amino acids 1-127 of SEQ ID NO:29 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:30 or a variant thereof comprising 1, 2, 3, or 4 conservative amino acid substitutions;
p) an antibody wherein the VH region comprises amino acids 1-127 of SEQ ID NO:31 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:32 or a variant thereof comprising 1, 2, 3, or 4 conservative amino acid substitutions;
q) an antibody wherein the VH region comprises amino acids 1-127 of SEQ ID NO:33 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:34 or a variant thereof comprising 1, 2, 3, or 4 conservative amino acid substitutions;
r) an antibody wherein the VH region comprises amino acids 1-120 of SEQ ID NO:35 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-100 of SEQ ID NO:36 or a variant thereof comprising 1, 2, 3, or 4 conservative amino acid substitutions;
s) an antibody wherein the VH region comprises amino acids 1-120 of SEQ ID NO:37 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-97 of SEQ ID NO:38 or a variant thereof comprising 1, 2, 3, or 4 conservative amino acid substitutions;
t) an antibody wherein the VH region comprises amino acids 1-123 of SEQ ID NO:39 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-100 of SEQ ID NO:40 or a variant thereof comprising 1, 2, 3, or 4 conservative amino acid substitutions;
u) an antibody wherein the VH region comprises amino acids 1-120 of SEQ ID NO:41 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-97 of SEQ ID NO:42 or a variant thereof comprising 1, 2, 3, or 4 conservative amino acid substitutions;
v) an antibody wherein the VH region comprises amino acids 1-120 of SEQ ID NO:43 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-97 of SEQ ID NO:44 or a variant thereof comprising 1, 2, 3, or 4 conservative amino acid substitutions;
w) an antibody wherein the VH region comprises amino acids 1-125 of SEQ ID NO:45 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-101 of SEQ ID NO:46 or a variant thereof comprising 1, 2, 3, or 4 conservative amino acid substitutions;
x) an antibody wherein the VH region comprises amino acids 1-125 of SEQ ID NO:47 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-101 of SEQ ID NO:48 or a variant thereof comprising 1, 2, 3, or 4 conservative amino acid substitutions;
y) an antibody wherein the VH region comprises amino acids 1-120 of SEQ ID NO:49 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-96 of SEQ ID NO:50 or a variant thereof comprising 1, 2, 3, or 4 conservative amino acid substitutions;
z) an antibody wherein the VH region comprises amino acids 1-120 of SEQ ID NO:51 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-97 of SEQ ID NO:52 or a variant thereof comprising 1, 2, 3, or 4 conservative amino acid substitutions;
aa) an antibody wherein the VH region comprises amino acids 1-121 of SEQ ID NO:53 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:54 or a variant thereof comprising 1, 2, 3, or 4 conservative amino acid substitutions;
bb) an antibody wherein the VH region comprises amino acids 1-121 of SEQ ID NO:55 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:56 or a variant thereof comprising 1, 2, 3, or 4 conservative amino acid substitutions;
cc) an antibody wherein the VH region comprises amino acids 1-121 of SEQ ID NO:57 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:58 or a variant thereof comprising 1, 2, 3, or 4 conservative amino acid substitutions;
dd) an antibody wherein the VH region comprises amino acids 1-121 of SEQ ID NO:59 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:60 or a variant thereof comprising 1, 2, 3, or 4 conservative amino acid substitutions;
ee) an antibody wherein the VH region comprises amino acids 1-120 of SEQ ID NO:61 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-98 of SEQ ID NO:62 or a variant thereof comprising 1, 2, 3, or 4 conservative amino acid substitutions;
ff) an antibody wherein the VH region comprises amino acids 1-121 of SEQ ID NO:63 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:64 or a variant thereof comprising 1, 2, 3, or 4 conservative amino acid substitutions;
gg) an antibody wherein the VH region comprises amino acids 1-121 of SEQ ID NO:65 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:66 or a variant thereof comprising 1, 2, 3, or 4 conservative amino acid substitutions;
hh) an antibody wherein the VH region comprises amino acids 1-120 of SEQ ID NO:67 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-96 of SEQ ID NO:68 or a variant thereof comprising 1, 2, 3, or 4 conservative amino acid substitutions;
ii) an antibody wherein the VH region comprises amino acids 1-120 of SEQ ID NO:69 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-96 of SEQ ID NO:70 or a variant thereof comprising 1, 2, 3, or 4 conservative amino acid substitutions;
jj) an antibody wherein the VH region comprises amino acids 1-120 of SEQ ID NO:71 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-96 of SEQ ID NO:72 or a variant thereof comprising 1, 2, 3, or 4 conservative amino acid substitutions;
kk) an antibody wherein the VH region comprises amino acids 1-125 of SEQ ID NO:73 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-101 of SEQ ID NO:74 or a variant thereof comprising 1, 2, 3, or 4 conservative amino acid substitutions;
ll) an antibody wherein the VH region comprises amino acids 1-120 of SEQ ID NO:75 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-97 of SEQ ID NO:76 or a variant thereof comprising 1, 2, 3, or 4 conservative amino acid substitutions;
mm) an antibody wherein the VH region comprises amino acids 1-128 of SEQ ID NO:153 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:155 or a variant thereof comprising 1, 2, 3, or 4 conservative amino acid substitutions;
nn) an antibody wherein the VH region comprises amino acids 1-128 of SEQ ID NO:157 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:159 or a variant thereof comprising 1, 2, 3, or 4 conservative amino acid substitutions;
oo) an antibody wherein the VH region comprises amino acids 1-127 of SEQ ID NO:161 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-97 of SEQ ID NO:163 or a variant thereof comprising 1, 2, 3, or 4 conservative amino acid substitutions;
pp) an antibody wherein the VH region comprises amino acids 1-127 of SEQ ID NO:165 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-97 of SEQ ID NO:167 or a variant thereof comprising 1, 2, 3, or 4 conservative amino acid substitutions;
qq) an antibody wherein the VH region comprises amino acids 1-127 of SEQ ID NO:169 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-97 of SEQ ID NO:171 or a variant thereof comprising 1, 2, 3, or 4 conservative amino acid substitutions;
rr) an antibody wherein the VH region comprises amino acids 1-127 of SEQ ID NO:173 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-97 of SEQ ID NO:175 or a variant thereof comprising 1, 2, 3, or 4 conservative amino acid substitutions;
ss) an antibody wherein the VH region comprises amino acids 1-127 of SEQ ID NO:177 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-97 of SEQ ID NO:179 or a variant thereof comprising 1, 2, 3, or 4 conservative amino acid substitutions;
tt) an antibody wherein the VH region comprises amino acids 1-127 of SEQ ID NO:181 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-97 of SEQ ID NO:183 or a variant thereof comprising 1, 2, 3, or 4 conservative amino acid substitutions;
uu) an antibody wherein the VH region comprises amino acids 1-127 of SEQ ID NO:185 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-97 of SEQ ID NO:187 or a variant thereof comprising 1, 2, 3, or 4 conservative amino acid substitutions;
vv) an antibody wherein the VH region comprises amino acids 1-127 of SEQ ID NO:189 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-97 of SEQ ID NO:191 or a variant thereof comprising 1, 2, 3, or 4 conservative amino acid substitutions;
ww) an antibody wherein the VH region comprises amino acids 1-127 of SEQ ID NO:193 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-97 of SEQ ID NO:195 or a variant thereof comprising 1, 2, 3, or 4 conservative amino acid substitutions;
xx) an antibody wherein the VH region comprises amino acids 1-127 of SEQ ID NO:197 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-97 of SEQ ID NO:199 or a variant thereof comprising 1, 2, 3, or 4 conservative amino acid substitutions;
yy) an antibody wherein the VH region comprises amino acids 1-127 of SEQ ID NO:201 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:203 or a variant thereof comprising 1, 2, 3, or 4 conservative amino acid substitutions;
zz) an antibody wherein the VH region comprises amino acids 1-127 of SEQ ID NO:205 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:207 or a variant thereof comprising 1, 2, 3, or 4 conservative amino acid substitutions;
aaa) an antibody wherein the VH region comprises amino acids 1-127 of SEQ ID NO:209 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-100 of SEQ ID NO:211 or a variant thereof comprising 1, 2, 3, or 4 conservative amino acid substitutions;
bbb) an antibody wherein the VH region comprises amino acids 1-127 of SEQ ID NO:213 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:215 or a variant thereof comprising 1, 2, 3, or 4 conservative amino acid substitutions;
ccc) an antibody wherein the VH region comprises amino acids 1-127 of SEQ ID NO:217 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-97 of SEQ ID NO:219 or a variant thereof comprising 1, 2, 3, or 4 conservative amino acid substitutions;
ddd) an antibody wherein the VH region comprises amino acids 1-127 of SEQ ID NO:221 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:223 or a variant thereof comprising 1, 2, 3, or 4 conservative amino acid substitutions;
eee) an antibody wherein the VH region comprises amino acids 1-128 of SEQ ID NO:225 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:227 or a variant thereof comprising 1, 2, 3, or 4 conservative amino acid substitutions;
fff) an antibody wherein the VH region comprises amino acids 1-127 of SEQ ID NO:229 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:231 or a variant thereof comprising 1, 2, 3, or 4 conservative amino acid substitutions;
ggg) an antibody wherein the VH region comprises amino acids 1-127 of SEQ ID NO:233 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:235 or a variant thereof comprising 1, 2, 3, or 4 conservative amino acid substitutions;
hhh) an antibody wherein the VH region comprises amino acids 1-127 of SEQ ID NO:237 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:239 or a variant thereof comprising 1, 2, 3, or 4 conservative amino acid substitutions;
iii) an antibody wherein the VH region comprises amino acids 1-127 of SEQ ID NO:241 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:243 or a variant thereof comprising 1, 2, 3, or 4 conservative amino acid substitutions;
jjj) an antibody wherein the VH region comprises amino acids 1-127 of SEQ ID NO:245 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:247 or a variant thereof comprising 1, 2, 3, or 4 conservative amino acid substitutions;
kkk) an antibody wherein the VH region comprises amino acids 1-127 of SEQ ID NO:249 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:251 or a variant thereof comprising 1, 2, 3, or 4 conservative amino acid substitutions;
lll) an antibody wherein the VH region comprises amino acids 1-127 of SEQ ID NO:253 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:255 or a variant thereof comprising 1, 2, 3, or 4 conservative amino acid substitutions;
mmm) an antibody wherein the VH region comprises amino acids 1-127 of SEQ ID NO:257 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:259 or a variant thereof comprising 1, 2, 3, or 4 conservative amino acid substitutions;
nnn) an antibody wherein the VH region comprises amino acids 1-127 of SEQ ID NO:261 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:263 or a variant thereof comprising 1, 2, 3, or 4 conservative amino acid substitutions;
ooo) an antibody wherein the VH region comprises amino acids 1-127 of SEQ ID NO:265 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:267 or a variant thereof comprising 1, 2, 3, or 4 conservative amino acid substitutions;
ppp) an antibody wherein the VH region comprises amino acids 1-127 of SEQ ID NO:269 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:271 or a variant thereof comprising 1, 2, 3, or 4 conservative amino acid substitutions;
qqq) an antibody wherein the VH region comprises amino acids 1-127 of SEQ ID NO:273 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:275 or a variant thereof comprising 1, 2, 3, or 4 conservative amino acid substitutions;
rrr) an antibody wherein the VH region comprises amino acids 1-127 of SEQ ID NO:277 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:279 or a variant thereof comprising 1, 2, 3, or 4 conservative amino acid substitutions;
sss) an antibody wherein the VH region comprises amino acids 1-127 of SEQ ID NO:281 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:283 or a variant thereof comprising 1, 2, 3, or 4 conservative amino acid substitutions;
ttt) an antibody wherein the VH region comprises amino acids 1-127 of SEQ ID NO:285 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:287 or a variant thereof comprising 1, 2, 3, or 4 conservative amino acid substitutions;
uuu) an antibody wherein the VH region comprises amino acids 1-127 of SEQ ID NO:289 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:291 or a variant thereof comprising 1, 2, 3, or 4 conservative amino acid substitutions;
vvv) an antibody wherein the VH region comprises amino acids 1-120 of SEQ ID NO:293 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-100 of SEQ ID NO:295 or a variant thereof comprising 1, 2, 3, or 4 conservative amino acid substitutions;
www) an antibody wherein the VH region comprises amino acids 1-120 of SEQ ID NO:297 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-100 of SEQ ID NO:299 or a variant thereof comprising 1, 2, 3, or 4 conservative amino acid substitutions;
xxx) an antibody wherein the VH region comprises amino acids 1-120 of SEQ ID NO:301 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-100 of SEQ ID NO:303 or a variant thereof comprising 1, 2, 3, or 4 conservative amino acid substitutions;
yyy) an antibody wherein the VH region comprises amino acids 1-123 of SEQ ID NO:305 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-100 of SEQ ID NO:307 or a variant thereof comprising 1, 2, 3, or 4 conservative amino acid substitutions;
zzz) an antibody wherein the VH region comprises amino acids 1-128 of SEQ ID NO:309 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-100 of SEQ ID NO:311 or a variant thereof comprising 1, 2, 3, or 4 conservative amino acid substitutions;
aaaa) an antibody wherein the VH region comprises amino acids 1-128 of SEQ ID NO:313 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-100 of SEQ ID NO:315 or a variant thereof comprising 1, 2, 3, or 4 conservative amino acid substitutions;
bbbb) an antibody wherein the VH region comprises amino acids 1-120 of SEQ ID NO:317 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-97 of SEQ ID NO:319 or a variant thereof comprising 1, 2, 3, or 4 conservative amino acid substitutions;
cccc) an antibody wherein the VH region comprises amino acids 1-123 of SEQ ID NO:321 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-100 of SEQ ID NO:323 or a variant thereof comprising 1, 2, 3, or 4 conservative amino acid substitutions;
dddd) an antibody wherein the VH region comprises amino acids 1-120 of SEQ ID NO:325 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-97 of SEQ ID NO:327 or a variant thereof comprising 1, 2, 3, or 4 conservative amino acid substitutions;
eeee) an antibody wherein the VH region comprises amino acids 1-120 of SEQ ID NO:329 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-97 of SEQ ID NO:331 or a variant thereof comprising 1, 2, 3, or 4 conservative amino acid substitutions;
ffff) an antibody wherein the VH region comprises amino acids 1-120 of SEQ ID NO:333 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-97 of SEQ ID NO:335 or a variant thereof comprising 1, 2, 3, or 4 conservative amino acid substitutions;
gggg) an antibody wherein the VH region comprises amino acids 1-125 of SEQ ID NO:337 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-101 of SEQ ID NO:339 or a variant thereof comprising 1, 2, 3, or 4 conservative amino acid substitutions;
hhhh) an antibody wherein the VH region comprises amino acids 1-125 of SEQ ID NO:341 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-101 of SEQ ID NO:343 or a variant thereof comprising 1, 2, 3, or 4 conservative amino acid substitutions;
iiii) an antibody wherein the VH region comprises amino acids 1-120 of SEQ ID NO:345 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-96 of SEQ ID NO:347 or a variant thereof comprising 1, 2, 3, or 4 conservative amino acid substitutions;
jjjj) an antibody wherein the VH region comprises amino acids 1-120 of SEQ ID NO:349 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-97 of SEQ ID NO:351 or a variant thereof comprising 1, 2, 3, or 4 conservative amino acid substitutions;
kkkk) an antibody wherein the VH region comprises amino acids 1-121 of SEQ ID NO:353 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:355 or a variant thereof comprising 1, 2, 3, or 4 conservative amino acid substitutions;
llll) an antibody wherein the VH region comprises amino acids 1-121 of SEQ ID NO:357 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:359 or a variant thereof comprising 1, 2, 3, or 4 conservative amino acid substitutions;
mmmm) an antibody wherein the VH region comprises amino acids 1-121 of SEQ ID NO:361 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:363 or a variant thereof comprising 1, 2, 3, or 4 conservative amino acid substitutions;
nnnn) an antibody wherein the VH region comprises amino acids 1-121 of SEQ ID NO:365 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:367 or a variant thereof comprising 1, 2, 3, or 4 conservative amino acid substitutions;
oooo) an antibody wherein the VH region comprises amino acids 1-120 of SEQ ID NO:369 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-98 of SEQ ID NO:371 or a variant thereof comprising 1, 2, 3, or 4 conservative amino acid substitutions;
pppp) an antibody wherein the VH region comprises amino acids 1-121 of SEQ ID NO:373 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:375 or a variant thereof comprising 1, 2, 3, or 4 conservative amino acid substitutions;
qqqq) an antibody wherein the VH region comprises amino acids 1-121 of SEQ ID NO:377 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-99 of SEQ ID NO:379 or a variant thereof comprising 1, 2, 3, or 4 conservative amino acid substitutions;
rrrr) an antibody wherein the VH region comprises amino acids 1-120 of SEQ ID NO:381 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-96 of SEQ ID NO:383 or a variant thereof comprising 1, 2, 3, or 4 conservative amino acid substitutions;
ssss) an antibody wherein the VH region comprises amino acids 1-120 of SEQ ID NO:385 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-96 of SEQ ID NO:387 or a variant thereof comprising 1, 2, 3, or 4 conservative amino acid substitutions;
tttt) an antibody wherein the VH region comprises amino acids 1-120 of SEQ ID NO:389 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-96 of SEQ ID NO:391 or a variant thereof comprising 1, 2, 3, or 4 conservative amino acid substitutions;
uuuu) an antibody wherein the VH region comprises amino acids 1-125 of SEQ ID NO:393 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); wherein the VL region comprises amino acids 1-101 of SEQ ID NO:395 or a variant thereof comprising 1, 2, 3, or 4 conservative amino acid substitutions;
vvvv) an antibody wherein the VH region comprises and amino acids 1-120 of SEQ ID NO:397 or a variant thereof comprising 1, 2, 3, or 4 conservative amino acid substitutions, except that the heavy chain amino acid sequence is modified whereby a part or all of the framework 3 region is replaced with an amino acid sequence selected from the group consisting of QLSQDPDDPDWG (SEQ ID NO:541) and LFSQDLYYPDRG (SEQ ID NO:542); and wherein the VL region comprises amino acids 1-97 of SEQ ID NO:399 or a variant thereof comprising 1, 2, 3, or 4 conservative amino acid substitutions;
wwww) an antibody wherein the VH region comprises and amino acids 1-134 of SEQ ID NO:501 or a variant thereof comprising 1, 2, 3, or 4 conservative amino acid substitutions, and wherein the VL region comprises amino acids 1-97 of SEQ ID NO:503 or a variant thereof comprising 1, 2, 3, or 4 conservative amino acid substitutions;
xxxx) an antibody wherein the VH region comprises and amino acids 1-127 of SEQ ID NO:505 or a variant thereof comprising 1, 2, 3, or 4 conservative amino acid substitutions, and wherein the VL region comprises amino acids 1-100 of SEQ ID NO:295 or a variant thereof comprising 1, 2, 3, or 4 conservative amino acid substitutions;
yyyy) an antibody wherein the VH region comprises and amino acids 1-127 of SEQ ID NO:507 or a variant thereof comprising 1, 2, 3, or 4 conservative amino acid substitutions, and wherein the VL region comprises amino acids 1-100 of SEQ ID NO:327 or a variant thereof comprising 1, 2, 3, or 4 conservative amino acid substitutions;
zzzz) an antibody wherein the VH region comprises and amino acids 1-127 of SEQ ID NO:509 or a variant thereof comprising 1, 2, 3, or 4 conservative amino acid substitutions, and wherein the VL region comprises amino acids 1-97 of SEQ ID NO:511 or a variant thereof comprising 1, 2, 3, or 4 conservative amino acid substitutions;
aaaaa) an antibody wherein the VH region comprises and amino acids 1-127 of SEQ ID NO:513 or a variant thereof comprising 1, 2, 3, or 4 conservative amino acid substitutions, and wherein the VL region comprises amino acids 1-97 of SEQ ID NO:515 or a variant thereof comprising 1, 2, 3, or 4 conservative amino acid substitutions;
bbbbb) wherein the VH region comprises and amino acids 1-127 of SEQ ID NO:517 or a variant thereof comprising 1, 2, 3, or 4 conservative amino acid substitutions, and wherein the VL region comprises amino acids 1-97 of SEQ ID NO:519 or a variant thereof comprising 1, 2, 3, or 4 conservative amino acid substitutions;
ccccc) an antibody wherein the VH region comprises and amino acids 1-127 of SEQ ID NO:521 or a variant thereof comprising 1, 2, 3, or 4 conservative amino acid substitutions, and wherein the VL region comprises amino acids 1-97 of SEQ ID NO:523 or a variant thereof comprising 1, 2, 3, or 4 conservative amino acid substitutions;
ddddd) an antibody wherein the VH region comprises and amino acids 1-127 of SEQ ID NO:525 or a variant thereof comprising 1, 2, 3, or 4 conservative amino acid substitutions, and wherein the VL region comprises amino acids 1-97 of SEQ ID NO:527 or a variant thereof comprising 1, 2, 3, or 4 conservative amino acid substitutions;
eeeee) an antibody wherein the VH region comprises and amino acids 1-127 of SEQ ID NO:529 or a variant thereof comprising 1, 2, 3, or 4 conservative amino acid substitutions, and wherein the VL region comprises amino acids 1-97 of SEQ ID NO:531 or a variant thereof comprising 1, 2, 3, or 4 conservative amino acid substitutions;
fffff) an antibody wherein the VH region comprises and amino acids 1-127 of SEQ ID NO:533 or a variant thereof comprising 1, 2, 3, or 4 conservative amino acid substitutions, and wherein the VL region comprises amino acids 1-97 of SEQ ID NO:535 or a variant thereof comprising 1, 2, 3, or 4 conservative amino acid substitutions; and
ggggg) an antibody wherein the VH region comprises and amino acids 1-127 of SEQ ID NO:537 or a variant thereof comprising 1, 2, 3, or 4 conservative amino acid substitutions, and wherein the VL region comprises amino acids 1-97 of SEQ ID NO:539 or a variant thereof comprising 1, 2, 3, or 4 conservative amino acid substitutions.

In some embodiments, the anti-HIV antibody is selected from the group consisting of:
a) an antibody comprising a heavy chain variable region, wherein the CDRs comprise amino acid sequences GYDFIDYV (SEQ ID NO:401), MNPSGGGT (SEQ ID NO:402) and VRDRANGSGRRRFESVNWFLDL (SEQ ID NO:403), wherein the heavy chain in the framework 3 region comprises an amino acid sequence selected from the group consisting of QLSQDPDDPDWG (SEQ ID NO:541) and LFSQDLYYPDRG (SEQ ID NO:542);
b) an antibody comprising a light chain variable region, wherein the CDRs comprise amino acid sequences HNY, DFN and WAFEN (SEQ ID NO:404), wherein the antibody comprises a heavy chain variable region, wherein the heavy chain in the framework 3 region comprises an amino acid sequence selected from the group consisting of QLSQDPDDPDWG (SEQ ID NO:541) and LFSQDLYYPDRG (SEQ ID NO:542);
c) an antibody comprising a heavy chain variable region, wherein the CDRs comprise amino acid sequences GYRFPDYI (SEQ ID NO:497), MNPMGGQV (SEQ ID NO:421) and VRDRSNGSGKRFESSNWFLDL (SEQ ID NO:441), wherein the heavy chain in the framework 3 region comprises an amino acid sequence selected from the group consisting of QLSQDPDDPDWG (SEQ ID NO:541) and LFSQDLYYPDRG (SEQ ID NO:542);
d) an antibody comprising a light chain variable region, wherein the CDRs comprise amino acid sequences HNL, DFN and WAYEA (SEQ ID NO:408), wherein the antibody comprises a heavy chain variable region, wherein the heavy chain in the framework 3 region comprises an amino acid sequence selected from the group consisting of QLSQDPDDPDWG (SEQ ID NO:541) and LFSQDLYYPDRG (SEQ ID NO:542);
e) an antibody comprising a heavy chain variable region, wherein the CDRs comprise amino acid sequences GYKFPDYI (SEQ ID NO:405), INPMGGQV (SEQ ID NO:406) and VRDRSNGSGRRFESSN (SEQ ID NO:407), wherein the heavy chain in the framework 3 region comprises an amino acid sequence selected from the group consisting of QLSQDPDDPDWG (SEQ ID NO:541) and LFSQDLYYPDRG (SEQ ID NO:542);
f) an antibody comprising a heavy chain variable region, wherein the CDRs comprise amino acid sequences GYTFTDYL (SEQ ID NO:409), MNPVYGQV (SEQ ID NO:410) and VRDTGDGSRRHFDSINWFLDL (SEQ ID NO:411), wherein the heavy chain in the framework 3 region comprises an amino acid sequence selected from the group consisting of QLSQDPDDPDWG (SEQ ID NO:541) and LFSQDLYYPDRG (SEQ ID NO:542);
g) an antibody comprising a light chain variable region, wherein the CDRs comprise amino acid sequences HNY, DFD and WAFEA (SEQ ID NO:412), wherein the antibody comprises a heavy chain variable region, wherein the heavy chain in the framework 3 region comprises an amino acid sequence selected from the group consisting of QLSQDPDDPDWG (SEQ ID NO:541) and LFSQDLYYPDRG (SEQ ID NO:542);
h) an antibody comprising a heavy chain variable region, wherein the CDRs comprise amino acid sequences GYTFTDYV (SEQ ID NO:413), IDPPYGQV (SEQ ID NO:414) and VRDRSNGWGKRFESSNWFLDL (SEQ ID NO:415), wherein the heavy chain in the framework 3 region comprises an amino acid sequence selected from the group consisting of QLSQDPDDPDWG (SEQ ID NO:541) and LFSQDLYYPDRG (SEQ ID NO:542);
i) an antibody comprising a heavy chain variable region, wherein the CDRs comprise amino acid sequences GYTFTDYV (SEQ ID NO:413), INPGYGQV (SEQ ID NO:431) and VRDRSNGWGKRFESSNWFLDL (SEQ ID NO:415), wherein the heavy chain in the framework 3 region comprises an amino acid sequence selected from the group consisting of QLSQDPDDPDWG (SEQ ID NO:541) and LFSQDLYYPDRG (SEQ ID NO:542);
j) an antibody comprising a heavy chain variable region, wherein the CDRs comprise amino acid sequences GYTFVDYF (SEQ ID NO:416), MDPLNGRP (SEQ ID NO:417) and VRDKSNGSGRRFDSSNWFLDL (SEQ ID NO:418), wherein the heavy chain in the framework 3 region comprises an amino acid sequence selected from the group consisting of QLSQDPDDPDWG (SEQ ID NO:541) and LFSQDLYYPDRG (SEQ ID NO:542);
k) an antibody comprising a light chain variable region, wherein the CDRs comprise amino acid sequences HNY, DFN and WAYDA (SEQ ID NO:419), wherein the antibody comprises a heavy chain variable region, wherein the heavy chain in the framework 3 region comprises an amino acid sequence selected from the group consisting of QLSQDPDDPDWG (SEQ ID NO:541) and LFSQDLYYPDRG (SEQ ID NO:542);
l) an antibody comprising a heavy chain variable region, wherein the CDRs comprise amino acid sequences GYTFSDYI (SEQ ID NO:420), MNPMGGQV (SEQ ID NO:421) and VRDRSNGSGKRFESSNWFLDL (SEQ ID NO:441), wherein the heavy chain in the framework 3 region comprises an amino acid sequence selected from the group consisting of QLSQDPDDPDWG (SEQ ID NO:541) and LFSQDLYYPDRG (SEQ ID NO:542);
m) an antibody comprising a light chain variable region, wherein the CDRs comprise amino acid sequences HNL, DFN and WAYEV (SEQ ID NO:422), wherein the antibody comprises a heavy chain variable region, wherein the heavy chain in the framework 3 region comprises an amino acid sequence selected from the group consisting of QLSQDPDDPDWG (SEQ ID NO:541) and LFSQDLYYPDRG (SEQ ID NO:542);
n) an antibody comprising a heavy chain variable region, wherein the CDRs comprise amino acid sequences GYTFIDYI (SEQ ID NO:423), IDPMNGRP (SEQ ID NO:424) and VRDKSNGSGKRFDSSNWFLDL (SEQ ID NO:425), wherein the heavy chain in the framework 3 region comprises an amino acid sequence selected from the group consisting of QLSQDPDDPDWG (SEQ ID NO:541) and LFSQDLYYPDRG (SEQ ID NO:542);
o) an antibody comprising a heavy chain variable region, wherein the CDRs comprise amino acid sequences GYTFTDYI (SEQ ID NO:426), MNPMGGRT (SEQ ID NO:427) and VRDKSNGSGKRFDSSNWFLDL (SEQ ID NO:425), wherein the heavy chain in the framework 3 region comprises an amino acid sequence selected from the group consisting of QLSQDPDDPDWG (SEQ ID NO:541) and LFSQDLYYPDRG (SEQ ID NO:542);
p) an antibody comprising a heavy chain variable region, wherein the CDRs comprise amino acid sequences GYTFVDYL (SEQ ID NO:428), MDPMNGRP (SEQ ID NO:429) and VRDKSGGSGKLFDSSNWFLDL (SEQ ID NO:430), wherein the heavy chain in the framework 3 region comprises an amino acid sequence selected from the group consisting of QLSQDPDDPDWG (SEQ ID NO:541) and LFSQDLYYPDRG (SEQ ID NO:542);
q) an antibody comprising a heavy chain variable region, wherein the CDRs comprise amino acid sequences GYTFTDYV (SEQ ID NO:413), MDPSYGQV (SEQ ID NO:432) and VRDRSHGSGRQFESSNWFLDL (SEQ ID NO:433), wherein the heavy chain in the framework 3 region comprises an amino acid sequence selected from the group consisting of QLSQDPDDPDWG (SEQ ID NO:541) and LFSQDLYYPDRG (SEQ ID NO:542);
r) an antibody comprising a heavy chain variable region, wherein the CDRs comprise amino acid sequences GYTFTDYV (SEQ ID NO:413), MDPSFGQM (SEQ ID NO:434) and VRDRSHGSGRLFESSNWFLDL (SEQ ID NO:435), wherein the heavy chain in the framework 3 region comprises an amino acid sequence selected from the group consisting of QLSQDPDDPDWG (SEQ ID NO:541) and LFSQDLYYPDRG (SEQ ID NO:542);
s) an antibody comprising a heavy chain variable region, wherein the CDRs comprise amino acid sequences GYRFTDYV (SEQ ID NO:436), MDPSFGRM (SEQ ID NO:437) and VRDRSHGSGRLFESSNWFLDL (SEQ ID NO:435), wherein the heavy chain in the framework 3 region comprises an amino acid sequence selected from the group consisting of QLSQDPDDPDWG (SEQ ID NO:541) and LFSQDLYYPDRG (SEQ ID NO:542);
t) an antibody comprising a heavy chain variable region, wherein the CDRs comprise amino acid sequences GYTFIDYV (SEQ ID NO:438), MDPTYGRM (SEQ ID NO:439) and VRDRSHGSGRLFESSNWFLDL (SEQ ID NO:435), wherein the heavy chain in the framework 3 region comprises an amino acid sequence selected from the group consisting of QLSQDPDDPDWG (SEQ ID NO:541) and LFSQDLYYPDRG (SEQ ID NO:542);
u) an antibody comprising a heavy chain variable region, wherein the CDRs comprise amino acid sequences GYRFLDYI (SEQ ID NO:440), MNPMGGQV (SEQ ID NO:421) and VRDRSNGSGKRFESSNWFLDL (SEQ ID NO:441), wherein the heavy chain in the framework 3 region comprises an amino acid sequence selected from the group consisting of QLSQDPDDPDWG (SEQ ID NO:541) and LFSQDLYYPDRG (SEQ ID NO:542);
v) an antibody comprising a heavy chain variable region, wherein the CDRs comprise amino acid sequences GYKFMDQL (SEQ ID NO:442), MNPTYGQV (SEQ ID NO:443) and ARGPSGENYPFHY (SEQ ID NO:444), wherein the heavy chain in the framework 3 region comprises an amino acid sequence selected from the group consisting of QLSQDPDDPDWG (SEQ ID NO:541) and LFSQDLYYPDRG (SEQ ID NO:542);
w) an antibody comprising a light chain variable region, wherein the CDRs comprise amino acid sequences RHII (SEQ ID NO:445), DDD and NTYEF (SEQ ID NO:446), wherein the antibody comprises a heavy chain variable region, wherein the heavy chain in the framework 3 region comprises an amino acid sequence selected from the group consisting of QLSQDPDDPDWG (SEQ ID NO:541) and LFSQDLYYPDRG (SEQ ID NO:542);
x) an antibody comprising a heavy chain variable region, wherein the CDRs comprise amino acid sequences GYNFVDSR (SEQ ID NO:447), INPLQGGV (SEQ ID NO:448) and ARGIDGKSYPFHF (SEQ ID NO:449), wherein the heavy chain in the framework 3 region comprises an amino acid sequence selected from the group consisting of QLSQDPDDPDWG (SEQ ID NO:541) and LFSQDLYYPDRG (SEQ ID NO:542);
y) an antibody comprising a light chain variable region, wherein the CDRs comprise amino acid sequences S, ESS and SILEF (SEQ ID NO:450), wherein the antibody comprises a heavy chain variable region, wherein the heavy chain in the framework 3 region comprises an amino acid sequence selected from the group consisting of QLSQDPDDPDWG (SEQ ID NO:541) and LFSQDLYYPDRG (SEQ ID NO:542);
z) an antibody comprising a heavy chain variable region, wherein the CDRs comprise amino acid sequences GYTFTTHHGHF (SEQ ID NO:500), MNPMTGQM (SEQ ID NO:462) and ARGDFGQNYPFHY (SEQ ID NO:463), wherein the heavy chain in the framework 3 region comprises an amino acid sequence selected from the group consisting of QLSQDPDDPDWG (SEQ ID NO:541) and LFSQDLYYPDRG (SEQ ID NO:542);
aa) an antibody comprising a light chain variable region, wherein the CDRs comprise amino acid sequences NRYL (SEQ ID NO:464), DDN and ASYER (SEQ ID NO:465), wherein the antibody comprises a heavy chain variable region, wherein the heavy chain in the framework 3 region comprises an amino acid sequence selected from the group consisting of QLSQDPDDPDWG (SEQ ID NO:541) and LFSQDLYYPDRG (SEQ ID NO:542);
bb) an antibody comprising a heavy chain variable region, wherein the CDRs comprise amino acid sequences GYNFMDQF (SEQ ID NO:466), MNPIYGQV (SEQ ID NO:467) and ARGPSGENYPFHY (SEQ ID NO:444), wherein the heavy chain in the framework 3 region comprises an amino acid sequence selected from the group consisting of QLSQDPDDPDWG (SEQ ID NO:541) and LFSQDLYYPDRG (SEQ ID NO:542);
cc) an antibody comprising a light chain variable region, wherein the CDRs comprise amino acid sequences RHII (SEQ ID NO:445), DDD and NTYEF (SEQ ID NO:446), wherein the antibody comprises a heavy chain variable region, wherein the heavy chain in the framework 3 region comprises an amino acid sequence selected from the group consisting of QLSQDPDDPDWG (SEQ ID NO:541) and LFSQDLYYPDRG (SEQ ID NO:542);
dd) an antibody comprising a heavy chain variable region, wherein the CDRs comprise amino acid sequences GYNFVDSR (SEQ ID NO:447), INPLHGGV (SEQ ID NO:468) and ARGIDGKSYPFHF (SEQ ID NO:449), wherein the heavy chain in the framework 3 region comprises an amino acid sequence selected from the group consisting of QLSQDPDDPDWG (SEQ ID NO:541) and LFSQDLYYPDRG (SEQ ID NO:542);
ee) an antibody comprising a heavy chain variable region, wherein the CDRs comprise amino acid sequences GYTFTKYF (SEQ ID NO:451), IHPRTGAV (SEQ ID NO:452) and ARGAFEADSYGSSYPFHH (SEQ ID NO:453), wherein the heavy chain in the framework 3 region comprises an amino acid sequence selected from the group consisting of QLSQDPDDPDWG (SEQ ID NO:541) and LFSQDLYYPDRG (SEQ ID NO:542);
ff) an antibody comprising a light chain variable region, wherein the CDRs comprise amino acid sequences GNYNP (SEQ ID NO:454), EDN and ASFEF (SEQ ID NO:455), wherein the antibody comprises a heavy chain variable region, wherein the heavy chain in the framework 3 region comprises an amino acid sequence selected from the group consisting of QLSQDPDDPDWG (SEQ ID NO:541) and LFSQDLYYPDRG (SEQ ID NO:542);
gg) an antibody comprising a heavy chain variable region, wherein the CDRs comprise amino acid sequences GYTFTKYT (SEQ ID NO:456), IHPRTGAV (SEQ ID NO:452) and ARGAFEADLSGPTYPFHH (SEQ ID NO:457), wherein the heavy chain in the framework 3 region comprises an amino acid sequence selected from the group consisting of QLSQDPDDPDWG (SEQ ID NO:541) and LFSQDLYYPDRG (SEQ ID NO:542);
hh) an antibody comprising a heavy chain variable region, wherein the CDRs comprise amino acid sequences GFNFIDSV (SEQ ID NO:458), IKPLRGAV (SEQ ID NO:459) and AKGAFRGGSPFGF (SEQ ID NO:460), wherein the heavy chain in the framework 3 region comprises an amino acid sequence selected from the group consisting of QLSQDPDDPDWG (SEQ ID NO:541) and LFSQDLYYPDRG (SEQ ID NO:542);
ii) an antibody comprising a light chain variable region, wherein the CDRs comprise amino acid sequences DVT and ASREF (SEQ ID NO:461), wherein the antibody comprises a heavy chain variable region, wherein the heavy chain in the framework 3 region comprises an amino acid sequence selected from the group consisting of QLSQDPDDPDWG (SEQ ID NO:541) and LFSQDLYYPDRG (SEQ ID NO:542);
jj) an antibody comprising a heavy chain variable region, wherein the CDRs comprise amino acid sequences GYTFTSYF (SEQ ID NO:469), INPLHGAV (SEQ ID NO:470) and TRGIVADGWPYGH (SEQ ID NO:471), wherein the heavy chain in the framework 3 region comprises an amino acid sequence selected from the group consisting of QLSQDPDDPDWG (SEQ ID NO:541) and LFSQDLYYPDRG (SEQ ID NO:542);
kk) an antibody comprising a light chain variable region, wherein the CDRs comprise amino acid sequences S, EGA and SSLQF (SEQ ID NO:472), wherein the antibody comprises a heavy chain variable region, wherein the heavy chain in the framework 3 region comprises an amino acid sequence selected from the group consisting of QLSQDPDDPDWG (SEQ ID NO:541) and LFSQDLYYPDRG (SEQ ID NO:542);
ll) an antibody comprising a heavy chain variable region, wherein the CDRs comprise amino acid sequences GFTFIDHI (SEQ ID NO:473), IKPLRGAV (SEQ ID NO:459) and CKAAAPEEAFPLQY (SEQ ID NO:474), wherein the heavy chain in the framework 3 region comprises an amino acid sequence selected from the group consisting of QLSQDPDDPDWG (SEQ ID NO:541) and LFSQDLYYPDRG (SEQ ID NO:542);
mm) an antibody comprising a light chain variable region, wherein the CDRs comprise amino acid sequences NVD, DNN and SSRTF (SEQ ID NO:475), wherein the antibody comprises a heavy chain variable region, wherein the heavy chain in the framework 3 region comprises an amino acid sequence selected from the group consisting of QLSQDPDDPDWG (SEQ ID NO:541) and LFSQDLYYPDRG (SEQ ID NO:542);
nn) an antibody comprising a heavy chain variable region, wherein the CDRs comprise amino acid sequences GFKFIDHI (SEQ ID NO:476), IKPLGGVA (SEQ ID NO:477) and CKAAAPDEAFPLEY (SEQ ID NO:478), wherein the heavy chain in the framework 3 region comprises an amino acid sequence selected from the group consisting of QLSQDPDDPDWG (SEQ ID NO:541) and LFSQDLYYPDRG (SEQ ID NO:542);
oo) an antibody comprising a light chain variable region, wherein the CDRs comprise amino acid sequences NVD, DNN and SSTTF (SEQ ID NO:479), wherein the antibody comprises a heavy chain variable region, wherein the heavy chain in the framework 3 region comprises an amino acid sequence selected from the group consisting of QLSQDPDDPDWG (SEQ ID NO:541) and LFSQDLYYPDRG (SEQ ID NO:542);
pp) an antibody comprising a heavy chain variable region, wherein the CDRs comprise amino acid sequences GFAFLDH (SEQ ID NO:480), VKTIGGVV (SEQ ID NO:481) and SKAAAPDEAFPLEF (SEQ ID NO:482), wherein the heavy chain in the framework 3 region comprises an amino acid sequence selected from the group consisting of QLSQDPDDPDWG (SEQ ID NO:541) and LFSQDLYYPDRG (SEQ ID NO:542);
qq) an antibody comprising a heavy chain variable region, wherein the CDRs comprise amino acid sequences GFAFLDHI (SEQ ID NO:486), VKTIGGVV (SEQ ID NO:481) and SKAAAPDEAFPLEF (SEQ ID NO:482), wherein the heavy chain in the framework 3 region comprises an amino acid sequence selected from the group consisting of QLSQDPDDPDWG (SEQ ID NO:541) and LFSQDLYYPDRG (SEQ ID NO:542);
rr) an antibody comprising a heavy chain variable region, wherein the CDRs comprise amino acid sequences GFKFTEYF (SEQ ID NO:483), LNPLRGAV (SEQ ID NO:484), ARAVFNEAFPFDY (SEQ ID NO:485), wherein the heavy chain in the framework 3 region comprises an amino acid sequence selected from the group consisting of QLSQDPDDPDWG (SEQ ID NO:541) and LFSQDLYYPDRG (SEQ ID NO:542);
ss) an antibody comprising a light chain variable region, wherein the CDRs comprise amino acid sequences VS, DGD and ASREF (SEQ ID NO:461), wherein the antibody comprises a heavy chain variable region, wherein the heavy chain in the framework 3 region comprises an amino acid sequence selected from the group consisting of QLSQDPDDPDWG (SEQ ID NO:541) and LFSQDLYYPDRG (SEQ ID NO:542);
tt) an antibody comprising a light chain variable region, wherein the CDRs comprise amino acid sequences NVD, DND and SSTTF (SEQ ID NO:479), wherein the antibody comprises a heavy chain variable region, wherein the heavy chain in the framework 3 region comprises an amino acid sequence selected from the group consisting of QLSQDPDDPDWG (SEQ ID NO:541) and LFSQDLYYPDRG (SEQ ID NO:542);
uu) an antibody comprising a heavy chain variable region, wherein the CDRs comprise amino acid sequences GFNFIDSV (SEQ ID NO:458), IKPLRGGV (SEQ ID NO:490) and AKGAFGGSSPFGF (SEQ ID NO:491), wherein the heavy chain in the framework 3 region comprises an amino acid sequence selected from the group consisting of QLSQDPDDPDWG (SEQ ID NO:541) and LFSQDLYYPDRG (SEQ ID NO:542);
vv) an antibody comprising a heavy chain variable region, wherein the CDRs comprise amino acid sequences GFKFIDSV (SEQ ID NO:487), IKPLGGAV (SEQ ID NO:488) and AKGAFGGGSPFGF (SEQ ID NO:489), wherein the heavy chain in the framework 3 region comprises an amino acid sequence selected from the group consisting of QLSQDPDDPDWG (SEQ ID NO:541) and LFSQDLYYPDRG (SEQ ID NO:542);
ww) an antibody comprising a heavy chain variable region, wherein the CDRs comprise amino acid sequences GFTFIKYT (SEQ ID NO:492), IHPRTGAV (SEQ ID NO:452) and ARGAFEADLYGPTYPFHH (SEQ ID NO:493), wherein the heavy chain in the framework 3 region comprises an amino acid sequence selected from the group consisting of QLSQDPDDPDWG (SEQ ID NO:541) and LFSQDLYYPDRG (SEQ ID NO:542);
xx) an antibody comprising a light chain variable region, wherein the CDRs comprise amino acid sequences GSYNP (SEQ ID NO:494), DDN and ASFEF (SEQ ID NO:455), wherein the antibody comprises a heavy chain variable region, wherein the heavy chain in the framework 3 region comprises an amino acid sequence selected from the group consisting of QLSQDPDDPDWG (SEQ ID NO:541) and LFSQDLYYPDRG (SEQ ID NO:542); and
yy) an antibody comprising a heavy chain variable region, wherein the CDRs comprise amino acid sequences GYNFVDSL (SEQ ID NO:495), INPLQGGV (SEQ ID NO:448) and ARGIDGNSYPFHF (SEQ ID NO:496), wherein the heavy chain in the framework 3 region comprises an amino acid sequence selected from the group consisting of QLSQDPDDPDWG (SEQ ID NO:541) and LFSQDLYYPDRG (SEQ ID NO:542);
zz) an antibody comprising a heavy chain variable region, wherein the CDRs comprise amino acid sequences GYNFMDQF (SEQ ID NO:466), MNPIWGQV (SEQ ID NO:543) and ARGPSGENYPFHY (SEQ ID NO:444), wherein the heavy chain in the framework 3 region comprises an amino acid sequence selected from the group consisting of QLSQDPDDPDWG (SEQ ID NO:541) and LFSQDLYYPDRG (SEQ ID NO:542);
aaa) an antibody comprising a heavy chain variable region, wherein the CDRs comprise amino acid sequences GYNFMDQF (SEQ ID NO:466), MNPIFGQV (SEQ ID NO:544) and ARGPSGENYPFHY (SEQ ID NO:444), wherein the heavy chain in the framework 3 region comprises an amino acid sequence selected from the group consisting of QLSQDPDDPDWG (SEQ ID NO:541) and LFSQDLYYPDRG (SEQ ID NO:542);
bbb) an antibody comprising a heavy chain variable region, wherein the CDRs comprise amino acid sequences GYDFMDQF (SEQ ID NO:545), MNPIYGQV (SEQ ID NO:467) and ARGPSGENYPFHY (SEQ ID NO:444), wherein the heavy chain in the framework 3 region comprises an amino acid sequence selected from the group consisting of QLSQDPDDPDWG (SEQ ID NO:541) and LFSQDLYYPDRG (SEQ ID NO:542); and
a) an antibody comprising a heavy chain variable region, wherein the CDRs comprise amino acid sequences GYDFIDQF (SEQ ID NO:546), MNPIYGQV (SEQ ID NO:467) and ARGPSGENYPFHY (SEQ ID NO:444), wherein the heavy chain in the framework 3 region comprises an amino acid sequence selected from the group consisting of QLSQDPDDPDWG (SEQ ID NO:541) and LFSQDLYYPDRG (SEQ ID NO:542).

In some embodiments, the anti-HIV antibody is selected from the group consisting of:
a) an antibody comprising a heavy chain variable region, wherein the CDRs comprise amino acid sequences GYDFIDYV (SEQ ID NO:401), MNPSGGGT (SEQ ID NO:402) and VRDRANGSGRRRFESVNWFLDL (SEQ ID NO:403), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences HNY, DFN and WAFEN (SEQ ID NO:404);
b) an antibody comprising a heavy chain variable region, wherein the CDRs comprise amino acid sequences GYRFPDYI (SEQ ID NO:497), MNPMGGQV (SEQ ID NO:421) and VRDRSNGSGKRFESSNWFLDL (SEQ ID NO:441), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences HNL, DFN and WAYEA (SEQ ID NO:408);
c) an antibody comprising a heavy chain variable region, wherein the CDRs comprise amino acid sequences GYKFPDYI (SEQ ID NO:405), INPMGGQV (SEQ ID NO:406) and VRDRSNGSGRRFESSN (SEQ ID NO:407), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences HNL, DFN and WAYEA (SEQ ID NO:408);
d) an antibody comprising a heavy chain variable region, wherein the CDRs comprise amino acid sequences GYTFTDYL (SEQ ID NO:409), MNPVYGQV (SEQ ID NO:410) and VRDTGDGSRRHFDSINWFLDL (SEQ ID NO:411), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences HNY, DFD and WAFEA (SEQ ID NO:412);
e) an antibody comprising a heavy chain variable region, wherein the CDRs comprise amino acid sequences GYTFTDYV (SEQ ID NO:413), IDPPYGQV (SEQ ID NO:414) and VRDRSNGWGKRFESSNWFLDL (SEQ ID NO:415), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences HNL, DFN and WAYEA (SEQ ID NO:408);
f) an antibody comprising a heavy chain variable region, wherein the CDRs comprise amino acid sequences GYTFTDYV (SEQ ID NO:413), INPGYGQV (SEQ ID NO:431) and VRDRSNGWGKRFESSNWFLDL (SEQ ID NO:415), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences HNL, DFN and WAYEA (SEQ ID NO:408);
g) an antibody comprising a heavy chain variable region, wherein the CDRs comprise amino acid sequences GYTFVDYF (SEQ ID NO:416), MDPLNGRP (SEQ ID NO:417) and VRDKSNGSGRRFDSSNWFLDL (SEQ ID NO:418), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences HNY, DFN and WAYDA (SEQ ID NO:419);
h) an antibody comprising a heavy chain variable region, wherein the CDRs comprise amino acid sequences GYTFSDYI (SEQ ID NO:420), MNPMGGQV (SEQ ID NO:421) and VRDRSNGSGKRFESSNWFLDL (SEQ ID NO:441), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences HNL, DFN and WAYEV (SEQ ID NO:422);
i) an antibody comprising a heavy chain variable region, wherein the CDRs comprise amino acid sequences GYTFIDYI (SEQ ID NO:423), IDPMNGRP (SEQ ID NO:424) and VRDKSNGSGKRFDSSNWFLDL (SEQ ID NO:425), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences HNY, DFN and WAYDA (SEQ ID NO:419);
j) an antibody comprising a heavy chain variable region, wherein the CDRs comprise amino acid sequences GYTFTDYI (SEQ ID NO:426), MNPMGGRT (SEQ ID NO:427) and VRDKSNGSGKRFDSSNWFLDL (SEQ ID NO:425), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences HNL, DFN and WAYEA (SEQ ID NO:408);
k) an antibody comprising a heavy chain variable region, wherein the CDRs comprise amino acid sequences GYTFVDYL (SEQ ID NO:428), MDPMNGRP (SEQ ID NO:429) and VRDKSGGSGKLFDSSNWFLDL (SEQ ID NO:430), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences HNY, DFN and WAYDA (SEQ ID NO:419);
l) an antibody comprising a heavy chain variable region, wherein the CDRs comprise amino acid sequences GYTFTDYV (SEQ ID NO:413), INPGYGQV (SEQ ID NO:431) and VRDRSNGWGKRFESSNWFLDL (SEQ ID NO:415), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences HNL, DFN and WAYEA (SEQ ID NO:408);
m) an antibody comprising a heavy chain variable region, wherein the CDRs comprise amino acid sequences GYTFTDYV (SEQ ID NO:413), MDPSYGQV (SEQ ID NO:432) and VRDRSHGSGRQFESSNWFLDL (SEQ ID NO:433), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences HNL, DFN and WAYEA (SEQ ID NO:408);
n) an antibody comprising a heavy chain variable region, wherein the CDRs comprise amino acid sequences GYTFTDYV (SEQ ID NO:413), MDPSFGQM (SEQ ID NO:434) and VRDRSHGSGRLFESSNWFLDL (SEQ ID NO:435), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences HNL, DFN and WAYEA (SEQ ID NO:408);
o) an antibody comprising a heavy chain variable region, wherein the CDRs comprise amino acid sequences GYRFTDYV (SEQ ID NO:436), MDPSFGRM (SEQ ID NO:437) and VRDRSHGSGRLFESSNWFLDL (SEQ ID NO:435), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences HNL, DFN and WAYEA (SEQ ID NO:408);
p) an antibody comprising a heavy chain variable region, wherein the CDRs comprise amino acid sequences GYTFIDYV (SEQ ID NO:438), MDPTYGRM (SEQ ID NO:439) and VRDRSHGSGRLFESSNWFLDL (SEQ ID NO:435), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences HNL, DFN and WAYEA (SEQ ID NO:408);
q) an antibody comprising a heavy chain variable region, wherein the CDRs comprise amino acid sequences GYRFLDYI (SEQ ID NO:440), MNPMGGQV (SEQ ID NO:421) and VRDRSNGSGKRFESSNWFLDL (SEQ ID NO:441), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences HNL, DFN and WAYEA (SEQ ID NO:408);
r) an antibody comprising a heavy chain variable region, wherein the CDRs comprise amino acid sequences GYKFMDQL (SEQ ID NO:442), MNPTYGQV (SEQ ID NO:443) and ARGPSGENYPFHY (SEQ ID NO:444), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences RHII (SEQ ID NO:445), DDD and NTYEF (SEQ ID NO:446);
s) an antibody comprising a heavy chain variable region, wherein the CDRs comprise amino acid sequences GYNFVDSR (SEQ ID NO:447), INPLQGGV (SEQ ID NO:448) and ARGIDGKSYPFHF (SEQ ID NO:449), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences S, ESS and SILEF (SEQ ID NO:450);
t) an antibody comprising a heavy chain variable region, wherein the CDRs comprise amino acid sequences GYTFTTHHGHF (SEQ ID NO:500), MNPMTGQM (SEQ ID NO:462) and ARGDFGQNYPFHY (SEQ ID NO:463), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences NRYL (SEQ ID NO:464), DDN and ASYER (SEQ ID NO:465);
u) an antibody comprising a heavy chain variable region, wherein the CDRs comprise amino acid sequences GYNFMDQF (SEQ ID NO:466), MNPIYGQV (SEQ ID NO:467) and ARGPSGENYPFHY (SEQ ID NO:444), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences RHII (SEQ ID NO:445), DDD and NTYEF (SEQ ID NO:446);
v) an antibody comprising a heavy chain variable region, wherein the CDRs comprise amino acid sequences GYNFVDSR (SEQ ID NO:447), INPLHGGV (SEQ ID NO:468) and ARGIDGKSYPFHF (SEQ ID NO:449), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences S, ESS and SILEF (SEQ ID NO:450);
w) an antibody comprising a heavy chain variable region, wherein the CDRs comprise amino acid sequences GYTFTKYF (SEQ ID NO:451), IHPRTGAV (SEQ ID NO:452) and ARGAFEADSYGSSYPFHH (SEQ ID NO:453), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences GNYNP (SEQ ID NO:454), EDN and ASFEF (SEQ ID NO:455);
x) an antibody comprising a heavy chain variable region, wherein the CDRs comprise amino acid sequences GYTFTKYT (SEQ ID NO:456), IHPRTGAV (SEQ ID NO:452) and ARGAFEADLSGPTYPFHH (SEQ ID NO:457), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences GNYNP (SEQ ID NO:454), EDN and ASFEF (SEQ ID NO:455);
y) an antibody comprising a heavy chain variable region, wherein the CDRs comprise amino acid sequences GFNFIDSV (SEQ ID NO:458), IKPLRGAV (SEQ ID NO:459) and AKGAFRGGSPFGF (SEQ ID NO:460), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences DVT and ASREF (SEQ ID NO:461);
z) an antibody comprising a heavy chain variable region, wherein the CDRs comprise amino acid sequences GYTFTSYF (SEQ ID NO:469), INPLHGAV (SEQ ID NO:470) and TRGIVADGWPYGH (SEQ ID NO:471), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences S, EGA and SSLQF (SEQ ID NO:472);
aa) an antibody comprising a heavy chain variable region, wherein the CDRs comprise amino acid sequences GFTFIDHI (SEQ ID NO:473), IKPLRGAV (SEQ ID NO:459) and CKAAAPEEAFPLQY (SEQ ID NO:474), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences NVD, DNN and SSRTF (SEQ ID NO:475);
bb) an antibody comprising a heavy chain variable region, wherein the CDRs comprise amino acid sequences GFKFIDHI (SEQ ID NO:476), IKPLGGVA (SEQ ID NO:477) and CKAAAPDEAFPLEY (SEQ ID NO:478), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences NVD, DNN and SSTTF (SEQ ID NO:479);
cc) an antibody comprising a heavy chain variable region, wherein the CDRs comprise amino acid sequences GFAFLDH (SEQ ID NO:480), VKTIGGVV (SEQ ID NO:481) and SKAAAPDEAFPLEF (SEQ ID NO:482), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences NVD, DNN and SSTTF (SEQ ID NO:479);
dd) an antibody comprising a heavy chain variable region, wherein the CDRs comprise amino acid sequences GFAFLDHI (SEQ ID NO:486), VKTIGGVV (SEQ ID NO:481) and SKAAAPDEAFPLEF (SEQ ID NO:482), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences NVD, DNN and SSTTF (SEQ ID NO:479);
ee) an antibody comprising a heavy chain variable region, wherein the CDRs comprise amino acid sequences GFKFTEYF (SEQ ID NO:483), LNPLRGAV (SEQ ID NO:484), ARAVFNEAFPFDY (SEQ ID NO:485), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences VS, DGD and ASREF (SEQ ID NO:461);
ff) an antibody comprising a heavy chain variable region, wherein the CDRs comprise amino acid sequences GFKFIDHI (SEQ ID NO:476), IKPLGGVA (SEQ ID NO:477) and CKAAAPDEAFPLEY (SEQ ID NO:478), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences NVD, DND and SSTTF (SEQ ID NO:479);
gg) an antibody comprising a heavy chain variable region, wherein the CDRs comprise amino acid sequences GFAFLDHI (SEQ ID NO:486), VKTIGGVV (SEQ ID NO:481) and SKAAAPDEAFPLEF (SEQ ID NO:482), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences NVD, DNN and SSTTF (SEQ ID NO:479);
hh) an antibody comprising a heavy chain variable region, wherein the CDRs comprise amino acid sequences GFNFIDSV (SEQ ID NO:458), IKPLRGGV (SEQ ID NO:490) and AKGAFGGSSPFGF (SEQ ID NO:491), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences DVT and ASREF (SEQ ID NO:461);
ii) an antibody comprising a heavy chain variable region, wherein the CDRs comprise amino acid sequences GFKFIDSV (SEQ ID NO:487), IKPLGGAV (SEQ ID NO:488) and AKGAFGGGSPFGF (SEQ ID NO:489), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences DVT and ASREF (SEQ ID NO:461);
jj) an antibody comprising a heavy chain variable region, wherein the CDRs comprise amino acid sequences GFNFIDSV (SEQ ID NO:458), IKPLRGGV (SEQ ID NO:490) and AKGAFGGSSPFGF (SEQ ID NO:491), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences DVT and ASREF (SEQ ID NO:461);
kk) an antibody comprising a heavy chain variable region, wherein the CDRs comprise amino acid sequences GFTFIKYT (SEQ ID NO:492), IHPRTGAV (SEQ ID NO:452) and ARGAFEADLYGPTYPFHH (SEQ ID NO:493), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences GSYNP (SEQ ID NO:494), DDN and ASFEF (SEQ ID NO:455);
ll) an antibody comprising a heavy chain variable region, wherein the CDRs comprise amino acid sequences GYNFVDSL (SEQ ID NO:495), INPLQGGV (SEQ ID NO:448) and ARGIDGNSYPFHF (SEQ ID NO:496), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein the CDRs comprise amino acid sequences S, ESS and SILEF (SEQ ID NO:450).

In some embodiments, the anti-HIV antibody is selected from the group consisting of:
a) an antibody comprising a heavy chain variable region, wherein CDR H1 comprises GYDFIDYV (SEQ ID NO:401), CDR H2 comprises MNPSGGGT (SEQ ID NO:402) and CDR H3 comprises VRDRANGSGRRRFESVNWFLDL (SEQ ID NO:403), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises HNY, CDR L2 comprises DFN and CDR L3 comprises WAFEN (SEQ ID NO:404);
b) an antibody comprising a heavy chain variable region, wherein CDR H1 comprises GYKFPDYI (SEQ ID NO:405), CDR H2 comprises INPMGGQV (SEQ ID NO:406) and CDR H3 comprises VRDRSNGSGRRFESSN (SEQ ID NO:407), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises HNL, CDR L2 comprises DFN and CDR L3 comprises WAYEA (SEQ ID NO:408);
c) an antibody comprising a heavy chain variable region, wherein CDR H1 comprises GYTFTDYL (SEQ ID NO:409), CDR H2 comprises MNPVYGQV (SEQ ID NO:410) and CDR H3 comprises VRDTGDGSRRHFDSINWFLDL (SEQ ID NO:411), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises HNY, CDR L2 comprises DFD and CDR L3 comprises WAFEA (SEQ ID NO:412);
d) an antibody comprising a heavy chain variable region, wherein CDR H1 comprises GYTFTDYV (SEQ ID NO:413), CDR H2 comprises IDPPYGQV (SEQ ID NO:414) and CDR H3 comprises VRDRSNGWGKRFESSNWFLDL (SEQ ID NO:415), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises HNL, CDR L2 comprises DFN and CDR L3 comprises WAYEA (SEQ ID NO:408);
e) an antibody comprising a heavy chain variable region, wherein CDR H1 comprises GYTFVDYF (SEQ ID NO:416), CDR H2 comprises MDPLNGRP (SEQ ID NO:417) and CDR H3 comprises VRDKSNGSGRRFDSSNWFLDL (SEQ ID NO:418), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises HNY, CDR L2 comprises DFN and CDR L3 comprises WAYDA (SEQ ID NO:419);
f) an antibody comprising a heavy chain variable region, wherein CDR H1 comprises GYTFSDYI (SEQ ID NO:420), CDR H2 comprises MNPMGGQV (SEQ ID NO:421) and CDR H3 comprises VRDRSNGSGKRFESSNWFLDL (SEQ ID NO:441), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises HNL, CDR L2 comprises DFN and CDR L3 comprises WAYEV (SEQ ID NO:422);
g) an antibody comprising a heavy chain variable region, wherein CDR H1 comprises GYTFIDYI (SEQ ID NO:423), CDR H2 comprises IDPMNGRP (SEQ ID NO:424) and CDR H3 comprises VRDKSNGSGKRFDSSNWFLDL (SEQ ID NO:425), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises HNY, CDR L2 comprises DFN and CDR L3 comprises WAYDA (SEQ ID NO:419);
h) an antibody comprising a heavy chain variable region, wherein CDR H1 comprises GYTFTDYI (SEQ ID NO:426), CDR H2 comprises MNPMGGRT (SEQ ID NO:427) and CDR H3 comprises VRDKSNGSGKRFDSSNWFLDL (SEQ ID NO:425), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises HNL, CDR L2 comprises DFN and CDR L3 comprises WAYEA (SEQ ID NO:408);
i) an antibody comprising a heavy chain variable region, wherein CDR H1 comprises GYTFVDYL (SEQ ID NO:428), CDR H2 comprises MDPMNGRP (SEQ ID NO:429) and CDR H3 comprises VRDKSGGSGKLFDSSNWFLDL (SEQ ID NO:430), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises HNY, CDR L2 comprises DFN and CDR L3 comprises WAYDA (SEQ ID NO:419);
j) an antibody comprising a heavy chain variable region, wherein CDR H1 comprises GYTFTDYV (SEQ ID NO:413), CDR H2 comprises INPGYGQV (SEQ ID NO:431) and CDR H3 comprises VRDRSNGWGKRFESSNWFLDL (SEQ ID NO:415), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises HNL, CDR L2 comprises DFN and CDR L3 comprises WAYEA (SEQ ID NO:408);
k) an antibody comprising a heavy chain variable region, wherein CDR H1 comprises GYTFTDYV (SEQ ID NO:413), CDR H2 comprises MDPSYGQV (SEQ ID NO:432) and CDR H3 comprises VRDRSHGSGRQFESSNWFLDL (SEQ ID NO:433), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises HNL, CDR L2 comprises DFN and CDR L3 comprises WAYEA (SEQ ID NO:408);
l) an antibody comprising a heavy chain variable region, wherein CDR H1 comprises GYTFTDYV (SEQ ID NO:413), CDR H2 comprises MDPSFGQM (SEQ ID NO:434) and CDR H3 comprises VRDRSHGSGRLFESSNWFLDL (SEQ ID NO:435), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises HNL, CDR L2 comprises DFN and CDR L3 comprises WAYEA (SEQ ID NO:408);
m) an antibody comprising a heavy chain variable region, wherein CDR H1 comprises GYRFTDYV (SEQ ID NO:436), CDR H2 comprises MDPSFGRM (SEQ ID NO:437) and CDR H3 comprises VRDRSHGSGRLFESSNWFLDL (SEQ ID NO:435), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises HNL, CDR L2 comprises DFN and CDR L3 comprises WAYEA (SEQ ID NO:408);
n) an antibody comprising a heavy chain variable region, wherein CDR H1 comprises GYTFIDYV (SEQ ID NO:438), CDR H2 comprises MDPTYGRM (SEQ ID NO:439) and CDR H3 comprises VRDRSHGSGRLFESSNWFLDL (SEQ ID NO:435), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises HNL, CDR L2 comprises DFN and CDR L3 comprises WAYEA (SEQ ID NO:408);
o) an antibody comprising a heavy chain variable region, wherein CDR H1 comprises GYRFLDYI (SEQ ID NO:440), CDR H2 comprises MNPMGGQV (SEQ ID NO:421) and CDR H3 comprises VRDRSNGSGKRFESSNWFLDL (SEQ ID NO:441), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises HNL, CDR L2 comprises DFN and CDR L3 comprises WAYEA (SEQ ID NO:408);
p) an antibody comprising a heavy chain variable region, wherein CDR H1 comprises GYKFMDQL (SEQ ID NO:442), CDR H2 comprises MNPTYGQV (SEQ ID NO:443) and CDR H3 comprises ARGPSGENYPFHY (SEQ ID NO:444), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises RHII (SEQ ID NO:445), CDR L2 comprises DDD and CDR L3 comprises NTYEF (SEQ ID NO:446);
q) an antibody comprising a heavy chain variable region, wherein CDR H1 comprises GYNFVDSR (SEQ ID NO:447), CDR H2 comprises INPLQGGV (SEQ ID NO:448) and CDR H3 comprises ARGIDGKSYPFHF (SEQ ID NO:449), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises S, CDR L2 comprises ESS and CDR L3 comprises SILEF (SEQ ID NO:450);
r) an antibody comprising a heavy chain variable region, wherein CDR H1 comprises GYTFTTHHGHF (SEQ ID NO:500), CDR H2 comprises MNPMTGQM (SEQ ID NO:462) and CDR H3 comprises ARGDFGQNYPFHY (SEQ ID NO:463), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises NRYL (SEQ ID NO:464), CDR L2 comprises DDN and CDR L3 comprises ASYER (SEQ ID NO:465);
s) an antibody comprising a heavy chain variable region, wherein CDR H1 comprises GYNFMDQF (SEQ ID NO:466), CDR H2 comprises MNPIYGQV (SEQ ID NO:467) and CDR H3 comprises ARGPSGENYPFHY (SEQ ID NO:444), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises RHII (SEQ ID NO:445), CDR L2 comprises DDD and CDR L3 comprises NTYEF (SEQ ID NO:446);
t) an antibody comprising a heavy chain variable region, wherein CDR H1 comprises GYNFVDSR (SEQ ID NO:447), CDR H2 comprises INPLHGGV (SEQ ID NO:468) and CDR H3 comprises ARGIDGKSYPFHF (SEQ ID NO:449), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises S, CDR L2 comprises ESS and CDR L3 comprises SILEF (SEQ ID NO:450);
u) an antibody comprising a heavy chain variable region, wherein CDR H1 comprises GYTFTKYF (SEQ ID NO:451), CDR H2 comprises IHPRTGAV (SEQ ID NO:452) and CDR H3 comprises ARGAFEADSYGSSYPFHH (SEQ ID NO:453), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises GNYNP (SEQ ID NO:454), CDR L2 comprises EDN and CDR L3 comprises ASFEF (SEQ ID NO:455);
v) an antibody comprising a heavy chain variable region, wherein CDR H1 comprises GYTFTKYT (SEQ ID NO:456), CDR H2 comprises IHPRTGAV (SEQ ID NO:452) and CDR H3 comprises ARGAFEADLSGPTYPFHH (SEQ ID NO:457), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises GNYNP (SEQ ID NO:454), CDR L2 comprises EDN and CDR L3 comprises ASFEF (SEQ ID NO:455);
w) an antibody comprising a heavy chain variable region, wherein CDR H1 comprises GFNFIDSV (SEQ ID NO:458), CDR H2 comprises IKPLRGAV (SEQ ID NO:459) and CDR H3 comprises AKGAFRGGSPFGF (SEQ ID NO:460), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises DVT and CDR L2 comprises ASREF (SEQ ID NO:461);
x) an antibody comprising a heavy chain variable region, wherein CDR H1 comprises GYTFTSYF (SEQ ID NO:469), CDR H2 comprises INPLHGAV (SEQ ID NO:470) and CDR H3 comprises TRGIVADGWPYGH (SEQ ID NO:471), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises S, CDR L2 comprises EGA and CDR L3 comprises SSLQF (SEQ ID NO:472);
y) an antibody comprising a heavy chain variable region, wherein CDR H1 comprises GFTFIDHI (SEQ ID NO:473), CDR H2 comprises IKPLRGAV (SEQ ID NO:459) and CDR H3 comprises CKAAAPEEAFPLQY (SEQ ID NO:474), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises NVD, CDR L2 comprises DNN and CDR L3 comprises SSRTF (SEQ ID NO:475);
z) an antibody comprising a heavy chain variable region, wherein CDR H1 comprises GFKFIDHI (SEQ ID NO:476), CDR H2 comprises IKPLGGVA (SEQ ID NO:477) and CDR H3 comprises CKAAAPDEAFPLEY (SEQ ID NO:478), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises NVD, CDR L2 comprises DNN and CDR L3 comprises SSTTF (SEQ ID NO:479);
aa) an antibody comprising a heavy chain variable region, wherein CDR H1 comprises GFAFLDH (SEQ ID NO:480), CDR H2 comprises VKTIGGVV (SEQ ID NO:481) and CDR H3 comprises SKAAAPDEAFPLEF (SEQ ID NO:482), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises NVD, CDR L2 comprises DNN and CDR L3 comprises SSTTF (SEQ ID NO:479);
bb) an antibody comprising a heavy chain variable region, wherein CDR H1 comprises GFKFTEYF (SEQ ID NO:483), CDR H2 comprises LNPLRGAV (SEQ ID NO:484) and CDR H3 comprises ARAVFNEAFPFDY (SEQ ID NO:485), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises VS, CDR L2 comprises DGD and CDR L3 comprises ASREF (SEQ ID NO:461);
cc) an antibody comprising a heavy chain variable region, wherein CDR H1 comprises GFKFIDHI (SEQ ID NO:476), CDR H2 comprises IKPLGGVA (SEQ ID NO:477) and CDR H3 comprises CKAAAPDEAFPLEY (SEQ ID NO:478), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises NVD, CDR L2 comprises DND and CDR L3 comprises SSTTF (SEQ ID NO:479);
dd) an antibody comprising a heavy chain variable region, wherein CDR H1 comprises GFAFLDHI (SEQ ID NO:486), CDR H2 comprises VKTIGGVV (SEQ ID NO:481) and CDR H3 comprises SKAAAPDEAFPLEF (SEQ ID NO:482), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises NVD, CDR L2 comprises DNN and CDR L3 comprises SSTTF (SEQ ID NO:479);
ee) an antibody comprising a heavy chain variable region, wherein CDR H1 comprises GFKFIDSV (SEQ ID NO:487), CDR H2 comprises IKPLGGAV (SEQ ID NO:488) and CDR H3 comprises AKGAFGGGSPFGF (SEQ ID NO:489), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises DVT and CDR L2 comprises ASREF (SEQ ID NO:461);
ff) an antibody comprising a heavy chain variable region, wherein CDR H1 comprises GFNFIDSV (SEQ ID NO:458), CDR H2 comprises IKPLRGGV (SEQ ID NO:490) and CDR H3 comprises AKGAFGGSSPFGF (SEQ ID NO:491), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises DVT and CDR L2 comprises ASREF (SEQ ID NO:461);
gg) an antibody comprising a heavy chain variable region, wherein CDR H1 comprises GFTFIKYT (SEQ ID NO:492), CDR H2 comprises IHPRTGAV (SEQ ID NO:452) and CDR H3 comprises ARGAFEADLYGPTYPFHH (SEQ ID NO:493), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises GSYNP (SEQ ID NO:494), CDR L2 comprises DDN and CDR L3 comprises ASFEF (SEQ ID NO:455);
hh) an antibody comprising a heavy chain variable region, wherein CDR H1 comprises GYNFVDSL (SEQ ID NO:495), CDR H2 comprises INPLQGGV (SEQ ID NO:448) and CDR H3 comprises ARGIDGNSYPFHF (SEQ ID NO:496), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises S, CDR L2 comprises ESS and CDR L3 comprises SILEF (SEQ ID NO:450);
ii) an antibody comprising a heavy chain variable region, wherein CDR H1 comprises GYRFPDYI (SEQ ID NO:497), CDR H2 comprises MNPTYGQV (SEQ ID NO:443) and CDR H3 comprises VRDRSNGSGKRFESSNWFLDL (SEQ ID NO:441), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises HNL, CDR L2 comprises DFN and CDR L3 comprises WAYEA (SEQ ID NO:408);
jj) an antibody comprising a heavy chain variable region, wherein CDR H1 comprises GYRFPDYI (SEQ ID NO:497), CDR H2 comprises MNPMGGQV (SEQ ID NO:421) and CDR H3 comprises VRDRSNGSGKRFESSNWFLDL (SEQ ID NO:441), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises HNY, CDR L2 comprises DFN and CDR L3 comprises WAFEN (SEQ ID NO:404);
kk) an antibody comprising a heavy chain variable region, wherein CDR H1 comprises GYRFPDYI (SEQ ID NO:497), CDR H2 comprises MNPMGGQV (SEQ ID NO:421) and CDR H3 comprises VRDRSNGSGKRFESSNWFLDL (SEQ ID NO:441), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises HNY, CDR L2 comprises DFD and CDR L3 comprises WAFEA (SEQ ID NO:412);
ll) an antibody comprising a heavy chain variable region, wherein CDR H1 comprises GYRFPDYI (SEQ ID NO:497), CDR H2 comprises MNPMGGQV (SEQ ID NO:421) and CDR H3 comprises VRDRSNGSGKRFESSNWFLDL (SEQ ID NO:441), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises RHII (SEQ ID NO:445), CDR L2 comprises DDD and CDR L3 comprises NTYEF (SEQ ID NO:446);
mm) an antibody comprising a heavy chain variable region, wherein CDR H1 comprises GYRFPDYI (SEQ ID NO:497), CDR H2 comprises MNPMGGQV (SEQ ID NO:421) and CDR H3 comprises VRDRSNGSGKRFESSNWFLDL (SEQ ID NO:441), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises HNY, CDR L2 comprises DFN and CDR L3 comprises WAYDA (SEQ ID NO:419);
nn) an antibody comprising a heavy chain variable region, wherein CDR H1 comprises GYRFPDYI (SEQ ID NO:497), CDR H2 comprises MNPMGGQV (SEQ ID NO:421) and CDR H3 comprises VRDRSNGSGKRFESSNWFLDL (SEQ ID NO:441), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises HNL, CDR L2 comprises DFN and CDR L3 comprises WAYEA (SEQ ID NO:408);
oo) an antibody comprising a heavy chain variable region, wherein CDR H1 comprises GYKFMDQL (SEQ ID NO:442), CDR H2 comprises MNPTYGQV (SEQ ID NO:443) and CDR H3 comprises VRDRSNGSGKRFESSN (SEQ ID NO:498), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises RHII (SEQ ID NO:445), CDR L2 comprises DDD and CDR L3 comprises NTYEF (SEQ ID NO:446);
pp) an antibody comprising a heavy chain variable region, wherein CDR H1 comprises GYKFMDQL (SEQ ID NO:442), CDR H2 comprises (SEQ ID NO:443) and CDR H3 comprises VRDRSNGSGKRFESSNWFLDL (SEQ ID NO:441), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises RHII (SEQ ID NO:445), CDR L2 comprises DDD and CDR L3 comprises NTYEF (SEQ ID NO:446); and
qq) an antibody comprising a heavy chain variable region, wherein CDR H1 comprises GYRFLDYI (SEQ ID NO:440), CDR H2 comprises MNPMGGQV (SEQ ID NO:421) and CDR H3 comprises VRDRSNGSGKRFESSNWFLDL (SEQ ID NO:441), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises HNL, CDR L2 comprises DFN and CDR L3 comprises WAYEA (SEQ ID NO:408).

In some embodiments, the anti-HIV antibody is selected from the group consisting of
a) an antibody comprising a heavy chain variable region, wherein CDR H1 comprises GYRFPDYI (SEQ ID NO:497), CDR H2 comprises MNPMGGQV (SEQ ID NO:421) and CDR H3 comprises VRDRSNGSGKRFESSNWFLDL (SEQ ID NO:441), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises HNL, CDR L2 comprises DFN and CDR L3 comprises WAYEA (SEQ ID NO:408);
b) an antibody comprising a heavy chain variable region, wherein CDR H1 comprises GYKFMDQL (SEQ ID NO:442), CDR H2 comprises MNPTYGQV (SEQ ID NO:443) and CDR H3 comprises ARGPSGENYPFHY (SEQ ID NO:444), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises RHII (SEQ ID NO:445), CDR L2 comprises DDD and CDR L3 comprises NTYEF (SEQ ID NO:446);
c) an antibody comprising a heavy chain variable region, wherein CDR H1 comprises GYNFMDQF (SEQ ID NO:466), CDR H2 comprises MNPIYGQV (SEQ ID NO:467) and CDR H3 comprises ARGPSGENYPFHY (SEQ ID NO:444), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises RHII (SEQ ID NO:445), CDR L2 comprises DDD and CDR L3 comprises NTYEF (SEQ ID NO:446);
d) an antibody comprising a heavy chain variable region, wherein CDR H1 comprises GYNFMDQF (SEQ ID NO:466), CDR H2 comprises MNPIWGQV (SEQ ID NO:543) and CDR H3 comprises ARGPSGENYPFHY (SEQ ID NO:444), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises RHII (SEQ ID NO:445), CDR L2 comprises DDD and CDR L3 comprises NTYEF (SEQ ID NO:446);
e) an antibody comprising a heavy chain variable region, wherein CDR H1 comprises GYNFMDQF (SEQ ID NO:466), CDR H2 comprises MNPIFGQV (SEQ ID NO:544) and CDR H3 comprises ARGPSGENYPFHY (SEQ ID NO:444), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises RHII (SEQ ID NO:445), CDR L2 comprises DDD and CDR L3 comprises NTYEF (SEQ ID NO:446);
f) an antibody comprising a heavy chain variable region, wherein CDR H1 comprises GYDFMDQF (SEQ ID NO:545), CDR H2 comprises MNPIYGQV (SEQ ID NO:467) and CDR H3 comprises ARGPSGENYPFHY (SEQ ID NO:444), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises RHII (SEQ ID NO:445), CDR L2 comprises DDD and CDR L3 comprises NTYEF (SEQ ID NO:446); and
g) an antibody comprising a heavy chain variable region, wherein CDR H1 comprises GYDFIDQF (SEQ ID NO:546), CDR H1 comprises MNPIYGQV (SEQ ID NO:467) and CDR H3 comprises ARGPSGENYPFHY (SEQ ID NO:444), wherein the heavy chain in the framework 3 region comprises an amino acid sequence selected from the group consisting of QLSQDPDDPDWG (SEQ ID NO:541) and LFSQDLYYPDRG (SEQ ID NO:542); and a light chain variable region, wherein CDR L1 comprises RHII (SEQ ID NO:445), CDR L2 comprises DDD and CDR L3 comprises NTYEF (SEQ ID NO:446).

In some embodiments, the anti-HIV antibody is a non-naturally occurring antibody. In some embodiments, the anti-HIV antibody is selected from the group consisting of: N49P6; N49P6.2; N49P7; N49P7.1; N49P7A; N49P7S; N49P7F; N49P7Y; N49P7-54TY; N49P7LS-1; N49P7LS-2; N49P7YTE; N49P7L6; N49P7L11; N49P7.1L9; N49P7.1L19 R49P7; N49P7.2; N49P11; N49P18; N49P18.2; N49P18.1; N49P19; N49P37; N49P38; N49P38.1; N49P55; N49P56; N49P57; N49P58; N49P59; N49P73; N49P74; N49P75; N49P75.1; N49P9; N49P9.1; N49P9.2; N49P9i7; N49P9i7H1; N49P9i7H2; N49P22; N49P23; N49P9.3; N49P9.4; N49P51; N49P52; N49P53; N49P54; N49P60; N49P61; N49P62; N49P63; N49P64; N49P65; N49P66; N49P67; N49P68; N49P69; N49P70; N49P71; and N49P72. In some embodiments, the heavy chain amino acid sequence of the above mentioned antibodies is modified whereby a part or all of the framework 3 region is replaced with an amino acid sequence selected from the group consisting of QLSQDPDDPDWG (SEQ ID NO:541) and LFSQDLYYPDRG (SEQ ID NO:542).

In some embodiments, the antibody is selected from Toripalimab, Retifanlimab, Elranatamab, Talquetamab, Glofitamab, Epcoritamab, Lecanemab, Rozanolixizumab, Pozelimab, Mirikizumab, Bimekizumab, Nirsevimab, Zolbetuximab-clzb, Zenocutuzumab-zbco, Zanidatamab-hrii, Datopotamab deruxtecan-dlnk, Muromonab-CD3, Rituximab, Adalimumab, Trastuzumab, Ado-trastuzumab emtansine, Casirivimab, Imdevimab, Gemtuzumab ozogamicin, Brentuximab Vedotin, and Inotuzumab ozogamicin.

### Methods

In one embodiment, the invention provides a method of treating or preventing a disease or condition in a subject, comprising administering subcutaneously to the subject an effective amount of the liposomal pharmaceutical formulation as provided herein. In some embodiments, the subject is a human.

In another embodiment, the invention provides a method of treating or preventing a disease or condition in a subject, comprising administering intravenously to the subject an effective amount of the liposomal pharmaceutical formulation as provided herein. In some embodiments, the subject is a human.

The disease or condition to be treated is not limiting. In some embodiments, the disease or condition is HIV infection. In some embodiments, an effective amount of one or more anti-HIV antibodies as described herein are administered to neutralize, treat, prevent or eradicate HIV infection.

Subjects at risk for HIV-related diseases or disorders include patients who have come into contact with an infected person or who have been exposed to HIV-1 in some other way. Administration of a prophylactic agent can occur prior to the manifestation of symptoms characteristic of HIV-1-related disease or disorder, such that a disease or disorder is prevented or, alternatively, delayed in its progression.

Methods for preventing an increase in HIV-1 virus titer, virus replication, virus proliferation or an amount of an HIV-1 viral protein in a subject are further provided. In one embodiment, a method includes administering to the subject an effective amount of the liposomal pharmaceutical formulation comprising an anti-HIV antibody to prevent an increase in HIV-1 titer, virus replication or an amount of an HIV-1 protein of one or more HIV strains or isolates in the subject.

In some embodiments, the antibody is Toripalimab and disease or condition is cancer.

In some embodiments, the antibody is Retifanlimab and disease or condition is cancer.

In some embodiments, the antibody is Elranatamab and disease or condition is Multiple myeloma.

In some embodiments, the antibody is Talquetamab and disease or condition is Multiple myeloma

In some embodiments, the antibody is Glofitamab and disease or condition is Diffuse large B-cell lymphoma.

In some embodiments, the antibody is Epcoritamab and disease or condition is Diffuse large B-cell lymphoma.

In some embodiments, the antibody is Lecanemab and disease or condition is Alzheimer's disease.

In some embodiments, the antibody is Rozanolixizumab and disease or condition is Myasthenia gravis.

In some embodiments, the antibody is Pozelimab and disease or condition is CHAPLE disease.

In some embodiments, the antibody is Mirikizumab and disease or condition is ulcerative colitis.

In some embodiments, the antibody is Bimekizumab and disease or condition is Plaque psoriasis.

In some embodiments, the antibody is Zolbetuximab-clzb and disease or condition is gastric cancer.

In some embodiments, the antibody is Zenocutuzumab-zbco and disease or condition is pancreatic or non-small cell lung cancer.

In some embodiments, the antibody is Nirsevimab and disease or condition is respiratory syncytial virus (RSV) prophylaxis in infants.

In some embodiments, the antibody is Zanidatamab-hrii and disease or condition is biliary tract cancer.

In some embodiments, the antibody is Datopotamab deruxtecan-dlnk and disease or condition is breast cancer.

In some embodiments, the antibody is Muromonab-CD3 and disease or condition is for prevention of organ transplant rejection.

In some embodiments, the antibody is Rituximab and disease or condition is Non-Hodgkin's lymphoma, chronic lymphocytic leukemia (CLL), or rheumatoid arthritis.

In some embodiments, the antibody is Adalimumab and disease or condition is Rheumatoid arthritis, Crohn's disease, ulcerative colitis, or plaque psoriasis.

In some embodiments, the antibody is Trastuzumab and disease or condition is HER2-positive breast cancer, or gastric cancer.

In some embodiments, the antibody is Ado-trastuzumab emtansine and disease or condition is HER2-positive metastatic breast cancer.

In some embodiments, the antibody is Casirivimab and/or Imdevimab and disease or condition is COVID-19.

In some embodiments, the antibody is Gemtuzumab ozogamicin and disease or condition is Acute myeloid leukemia.

In some embodiments, the antibody is Brentuximab Vedotin and disease or condition is Hodgkin lymphoma, systemic anaplastic large cell lymphoma.

In some embodiments, the antibody is Inotuzumab ozogamicin and disease or condition is Acute lymphoblastic leukemia.

The dose and dosage regimen of the antibody depends upon a variety of factors readily determined by a physician, such as the nature of the infection and the characteristics of the particular cytotoxic agent or growth inhibitory agent conjugated to the antibody (when used), e.g., its therapeutic index, the patient, and the patient's history. Generally, a therapeutically effective amount of an antibody is administered to a patient. In particular embodiments, the amount of antibody administered is in the range of about 0.1 mg/kg to about 20 mg/kg of patient body weight. Depending on the type and severity of the infection, about 0.1 mg/kg to about 20 mg/kg body weight (e.g., about 0.1-15 mg/kg/dose) of antibody is an initial candidate dosage for administration to the patient, whether, for example, by one or more separate administrations. The progress of this therapy is readily monitored by conventional methods and assays and based on criteria known to the physician or other persons of skill in the art.

Antibodies described herein can be coupled to a drug for delivery to a treatment site or coupled to a detectable label to facilitate imaging of a site comprising cells of interest, such as cells infected with HIV. Methods for coupling antibodies to drugs and detectable labels are well known in the art, as are methods for imaging using detectable labels. Labeled antibodies may be employed in a wide variety of assays, employing a wide variety of labels. Detection of the formation of an antibody-antigen complex between an antibody of the invention and an epitope of interest (an HIV epitope) can be facilitated by attaching a detectable substance to the antibody. Suitable detection means include the use of labels such as radionucleotides, enzymes, coenzymes, fluorescers, chemiluminescers, chromogens, enzyme substrates or co-factors, enzyme inhibitors, prosthetic group complexes, free radicals, particles, dyes, and the like. Examples of suitable enzymes include horseradish peroxidase, alkaline phosphatase, Q-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material is luminol; examples of bioluminescent materials include luciferase, luciferin, and aequorin; and examples of suitable radioactive material include ¹²⁵I, ¹³¹I, ³S, or .sup.3H. Such labeled reagents may be used in a variety of well-known assays, such as radioimmunoassays, enzyme immunoassays, e.g., ELISA, fluorescent immunoassays, and the like.

The antibodies can be tagged with such labels by known methods. For instance, coupling agents such as aldehydes, carbodiimides, dimaleimide, imidates, succinimides, bid-diazotized benzadine and the like are used to tag the antibodies with the above-described fluorescent, chemiluminescent, and enzyme labels. An enzyme is typically combined with an antibody using bridging molecules such as carbodiimides, periodate, diisocyanates, glutaraldehyde and the like. Various labeling techniques are described in Morrison, Methods in Enzymology 32b, 103 (1974), Syvanen et al., J. Biol. Chem. 284, 3762 (1973) and Bolton and Hunter, Biochem J. 133, 529(1973).

In one embodiment, the antibodies can be immunoconjugates, conjugated to a second molecule. For example, the second molecule can be a toxin, a label, a radioisotope, a drug, or a chemical compound.

An antibody according to the invention may be conjugated to a therapeutic moiety such as a cytotoxin, a therapeutic agent, or a radioactive metal ion or radioisotope. Examples of radioisotopes include, but are not limited to, I-131, I-123, I-125, Y-90, Re-188, Re-186, At-211, Cu-67, Bi-212, Bi-213, Pd-109, Tc-99, In-111, and the like. Such antibody conjugates can be used for modifying a given biological response; the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin, TLR agonists (such as TLR7 agonist), or monomethylauristatin E.

Other therapeutic regimens can be combined with the administration of the anti-HIV antibody of the present invention. The combined administration includes co-administration, using separate formulations or a single pharmaceutical formulation, and consecutive administration in either order, wherein preferably there is a time period while both (or all) active agents simultaneously exert their biological activities. Preferably such combined therapy results in a synergistic therapeutic effect.

For any application, the liposomal pharmaceutical formulation can be combined with anti-retroviral therapy. Antiretroviral drugs are broadly classified by the phase of the retrovirus life-cycle that the drug inhibits. The disclosed antibodies can be administered in conjunction with nucleoside analog reverse-transcriptase inhibitors (such as zidovudine, didanosine, zalcitabine, stavudine, lamivudine, abacavir, emtricitabine, entecavir, and apricitabine), nucleotide reverse transcriptase inhibitors (such as tenofovir and adefovir), non-nucleoside reverse transcriptase inhibitors (such as efavirenz, nevirapine, delavirdine, etravirine, and rilpivirine), protease inhibitors (such as saquinavir, ritonavir, indinavir, nelfinavir, amprenavir, lopinavir, fosamprenavir, atazanavir, tipranavir, and darunavir), entry or fusion inhibitors (such as maraviroc and enfuvirtide), maturation inhibitors, (such as bevirimat and vivecon), or a broad spectrum inhibitors, such as natural antivirals. In some embodiments, a disclosed antibody or active fragment thereof is administered in conjunction with IL-15, or conjugated to IL-15.

In some embodiments, the liposomal pharmaceutical formulation of the antibodies injected subcutaneously or intravenously increase the AUC, bioavailability, and half-life of the antibody compared to free antibody injected subcutaneously or intravenously. In some embodiments the liposomal pharmaceutical formulation has reduced immunogenicity with less generation of anti-drug antibodies.

In some embodiments, release of the antibody into the subject's plasma is delayed and then sustained over a period of time. In some embodiments, the antibody achieves a peak plasma concentration at least 7 days following administration in the subject. In some embodiments, the antibody achieves a peak plasma concentration at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, or at least 21 days, following administration in the subject. In some embodiments, the antibody achieves a peak plasma concentration between 7 and 14 days following administration in the subject.

In some embodiments, the antibody has a half-life of from 12 days to several months, e.g., three months. In some embodiments, the antibody has a half-life of at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, at least 31 days, at least 32 days, at least 33 days, at least 34 days, at least 35 days, at least 36 days, at least 37 days, at least 38 days, at least 39 days, at least 40 days, at least 41 days, at least 42 days, at least 43 days, at least 44 days, at least 45 days, at least 46 days, at least 47 days, at least 48 days, at least 49 days, at least 50 days, at least 60 days, at least 70 days, at least 80 days, at least 90 days, or at least 100 days.

In some embodiments, the antibody has a half-life in the subject that is from about 30% to about 400% greater than the half-life of the free antibody in a control subject administered an intravenous injection of the free antibody. In some embodiments, the antibody has a half-life in the subject that is at least 30% greater, at least 40% greater, at least 50% greater, at least 60% greater, at least 70% greater, at least 80% greater, at least 90% greater, at least 100% greater, at least 110% greater, at least 120% greater, at least 130% greater, at least 140% greater, at least 150% greater, at least 160% greater, at least 170% greater, at least 180% greater, at least 190% greater, at least 200% greater, at least 210% greater, at least 220% greater, at least 230% greater, at least 240% greater, at least 250% greater, at least 260% greater, at least 270% greater, at least 280% greater, at least 290% greater, at least 300% greater, at least 310% greater, at least 320% greater, at least 330% greater, at least 340% greater, at least 350% greater, at least 360% greater, at least 370% greater, at least 380% greater, at least 390% greater, or at least 400% greater, than the half-life of the free antibody in a normal, control subject administered an intravenous injection of the free antibody.

In some embodiments, the liposomal pharmaceutical formulation of the antibodies injected subcutaneously or intravenously increase the AUC by at least about 30% to at least about 300% compared with injection intravenously or subcutaneously of free antibody. In some embodiments, AUC is increased by at least 30%, at least 40%, at least 50%, at least 60%, at least 70%, at least 80%, at least 90%, at least 100%, at least 110%, at least 120%, at least 130%, at least 140%, at least 150%, at least 160%, at least 170%, at least 180%, at least 190%, at least 200%, at least 210%, at least 220%, at least 230%, at least 240%, at least 250%, at least 260%, at least 270%, at least 280%, at least 290%, or at least 300%.

In some embodiments, the liposomal pharmaceutical formulation is administered in combination with another therapy. The additional therapy is not limiting. In some embodiments, the additional therapy is an anti-retroviral therapy.

For the treatment or prevention of HIV, the appropriate dosage of an antibody or combination of antibodies can depend on a variety of factors, such as the severity and course of the disease, the responsiveness of the disease, whether the antibody or agent is administered for therapeutic or preventative purposes, previous therapy, patient's clinical history, and so on all at the discretion of the treating physician. The antibody or agent can be administered one time or over a series of treatments lasting from several days to several months, or until a cure is effected or a diminution of the disease state is achieved. The administering physician can easily determine optimum dosages, dosing methodologies and repetition rates. In certain embodiments, dosage is from 0.01 g to 100 mg per kg of body weight, and can be given once or more daily, weekly, monthly or yearly. In certain embodiments, the antibody or combination of antibodies is given once every two weeks or once every three weeks. In certain embodiments, the dosage of the antibody is from about 0.1 mg to about 20 mg per kg of body weight. The treating physician can estimate repetition rates for dosing based on measured residence times and concentrations of the drug in bodily fluids or tissues.

The compositions of the invention can be administered alone, or can be co-administered, or sequentially administered, with other HIV immunogens and/or HIV immunogenic compositions, e.g., with “other” immunological, antigenic or vaccine or therapeutic compositions thereby providing multivalent or “cocktail” or combination compositions of the invention and methods of employing them. Again, the ingredients and manner (sequential or co-administration) of administration, as well as dosages can be determined taking into consideration such factors as the age, sex, weight, species and condition of the particular subject, and the route of administration.

Application of the teachings of the present invention to a specific problem is within the capabilities of one having ordinary skill in the art in light of the teaching contained herein. Examples of the compositions and methods of the invention appear in the following non-limiting Examples.

### EXAMPLES

### Example 1. Improving Monoclonal Antibody Pharmacokinetics Via Subcutaneous Liposomal Delivery

The present example describes development and testing of liposomal pharmaceutical formulations of antibodies for subcutaneous delivery.

### Physicochemical Characterization of Liposomes

In this study, we formulated two distinct liposomal compositions: HSPC:Chol:DSPE-mPEG2000 (molar ration of 50:35:5:10) DSPG and HSPC:Chol:DSPG (molar ratio of 60:20:20), with a lipid concentration of 100 mM (Table 4).

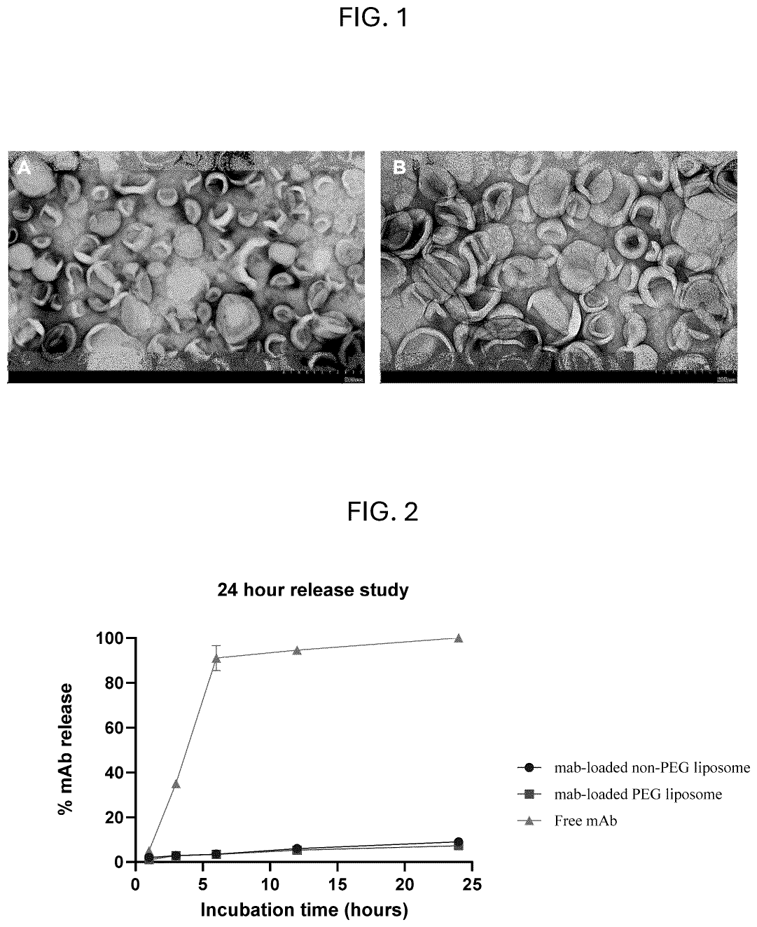

The encapsulation efficiency of each batch of mAbs, determined through BCA assay, between 58% and 61%. The characteristic features have been summarized particle characteristics, including size (nm), polydispersity index (PDI), and zeta potential (mV) in Table 4. The liposomal formulations exhibited sizes ranging from 85 to 92 nm, with a negative zeta potential due to the presence of the negatively charged lipid DSPG, promoting stability by preventing aggregation. Encapsulation efficiency was 61% and 58% for PEGylated and non-PEGylated liposomes, respectively. The homogeneity of the liposomal formulations was confirmed by a PDI below 0.2. TEM images (FIG. 1) provided visual confirmation of the liposomes' uniform and spherical shape, consistent with the size data in Table 4. It is crucial to emphasize that the physicochemical attributes of liposomal drug delivery, such as lipid content, size, and surface charge, play a pivotal role in shaping the resulting immune response (29).

TABLE-4
Physiochemical characteristics of liposomal formulations of N49P9.6-FR-LS

| | | | | Particle | Zeta | | |
|---|---|---|---|---|---|---|---|
| | | | | size | Potential [symbol] | | |
| | Lipid | Lipid | Molar | (nm +/− | (mV) +/− | PDI [symbol] | EE [symbol] |
| mAb | composition | PEGylation | ratio | SD [symbol] ) | SD | SD | % |
| N49P9.6.FR-LS | HSPC/Chol/ | Yes | 50:35:5:10 | 92 +/− | −14.5 +/− | 0.14 +/− | 61 |
| | DSPE- | | | 3.6 | 2.7 | 0.039 | |
| | mPEG2000/DSPG | | | | | | |
| N49P9.6-FR-LS | HSPC/Chol/ | No | 60:20:20 | 85 +/− | −42 +/− | 0.18 +/− | 58 |
| | DSPG | | | 5.8 | 3.7 | 0.059 | |

a [symbol] Standard Deviation
b [symbol] Surface Charge
c [symbol] Polydispersity Index
d [symbol] Enc [symbol] tion Efficacy [symbol]
indicates data missing or illegible when filed

### Stability Tests

Herein, we conducted a comprehensive evaluation of the physical stability of two distinct liposomal formulations, namely PEG-liposome and non-PEG liposome, over a storage period of 16 weeks at temperatures of 4 and 37° C. We evaluated various parameters, including appearance, size, zeta potential, polydispersity index (PDI), and encapsulation efficiency (EE %). As summarized in Table 5, both PEG-liposome and non-PEG liposome formulations exhibited minor changes in EE %, zeta potential, and average diameter during storage at 4° C., indicating stability with no observable alterations in appearance.

However, a different scenario unfolded over four months during storage at 37° C. The average diameter increased from 89.8±4.9 to 153.9±3.9 nm for PEG-liposome and from 95.4±9.5 to 168.7±4.7 nm for non-PEG liposome formulations. While the appearance of liposomes remained stable at 4° C., the elevated temperature of 37° C. led to substantial impacts on formulation leakage.

TABLE 5
Stability of N49P9.6-FR-LS-loaded liposomes at 4° C. and 37° C.

| | PEG liposome 4° C. | | | Non-PEG liposome 4° C. | | |
|---|---|---|---|---|---|---|
| Month | 0 | 2 | 4 | 0 | 2 | 4 |
| Size +/− SD^{a} | 89.8 +/− | 102.4 +/− | 124.9 +/− | 95.4 +/− | 100.4 +/− | 115.7 +/− |
| | 4.9 | 3.5 | 3.4 | 9.5 | 1.8 | 3.2 |
| Zeta Potential^{b} | −21.3 +/− | −25.7 +/− | −28 +/− | −6.92 +/− | −8.5 +/− | −9.7 +/− |
| (mV) +/− SD | 2.4 | 3.9 | 1.3 | 5.3 | 3.1 | 6.2 |
| PDI^{c} +/− SD | 0.13 +/− | 0.35 +/− | 0.5 +/− | 0.6 +/− | 0.2 +/− | 0.4 +/− |
| | 0.03 | 0.07 | 0.04 | 0.09 | 0.07 | 0.01 |
| EE^{d} % | 61 | 50 | 48 | 58 | 48 | 40 |
| | PEG liposome 37° C. | | | Non−PEG liposome 37° C. | | |
| Size +/− SD | 89.8 +/− | 128.4 +/− | 153.9 +/− | 95.4 +/− | 129.8 +/− | 168.7 +/− |
| | 4.9 | 2.5 | 3.9 | 9.5 | 2.3 | 4.7 |
| Zeta Potential | −21.3 +/− | −24.7 +/− | −27 +/− | −6.92 +/− | −9.3 +/− | −11 +/− |
| (mV) +/− SD | 2.4 | 1.3 | 9.6 | 5.3 | 8.4 | 4.6 |
| PDI +/− SD | 0.13 +/− | 0.58 +/− | 0.89 +/− | 0.6 +/− | 0.81 +/− | 0.87 +/− |
| | 0.03 | 0.01 | 0.09 | 0.09 | 0.03 | 0.05 |
| EE % | 61 | 41 | 32 | 58 | 37 | 29 |

^{a}Standard Deviation
^{b}Surface Charge
^{c}Polydispersity Index
^{d} Encapsulation Efficacy

By the end of the storage period (4 months), approximately 50% of the mAbs had been released from the liposomes (EE % of N49P9.6-FR from PEG-liposome and non-PEG liposome formulations decreased from 61% to 32% and 58% to 29%, respectively). Despite these changes in size and EE %, both PEG-liposome and non-PEG liposome formulations displayed no noticeable appearance changes over 16 weeks at both 4° and 37° C.

### Release Study

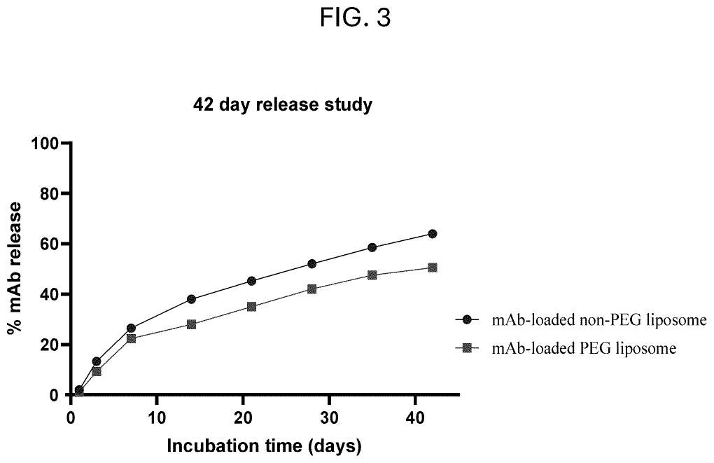

The characteristics of the in vitro release profiles for PEG-liposomes, non-PEG liposomes, and free mAb are depicted in FIG. 2. The results highlight a significantly lower release of mAb from both PEG-liposome and non-PEG liposomal formulations compared to free mAbs in 50% sterile human plasma in 5% sterilized dextrose (pH 7.4). Free mAbs exhibited a rapid diffusion rate, with 90% released within six hours from the dialysis bag. In contrast, the nanoliposomes released only 7-9% of mAbs after 24 hours, indicating a slower release than free mAbs. We investigated the stability and integrity of mAb-loaded liposomes under in vitro release conditions at a pH of 7.4, as illustrated in FIG. 3, which demonstrates that both PEG-liposomes and non-PEG liposomes exhibited sustained release properties without a burst release. After 42 days, the release percentages from PEG liposomes and non-PEG liposomes were approximately 51% and 65%, respectively. Notably, from this point until the end of the assay, the percentage of antibodies released from PEGylated liposomes was statistically lower than that from non-PEGylated liposomes (P value <0.0001).

### In Vitro Cytotoxic Studies

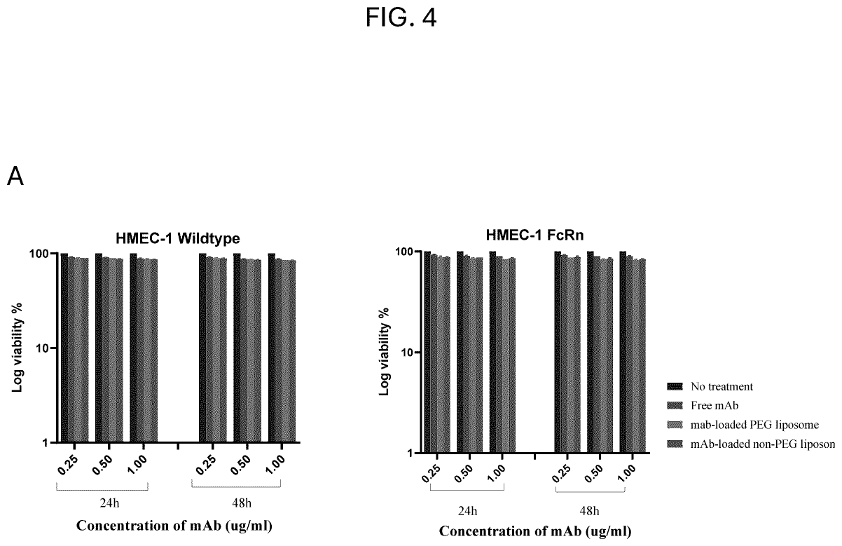

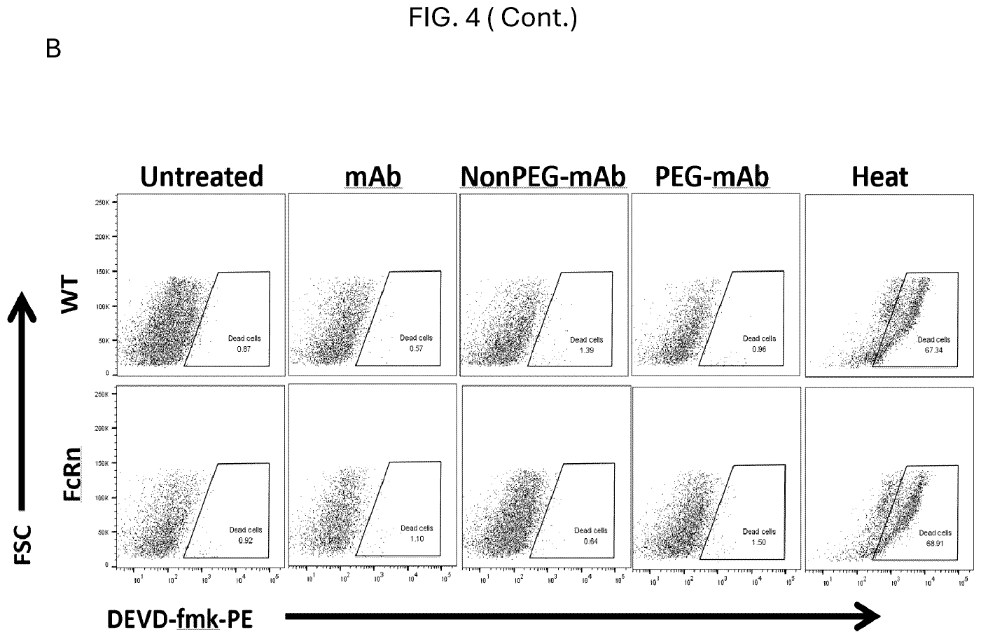

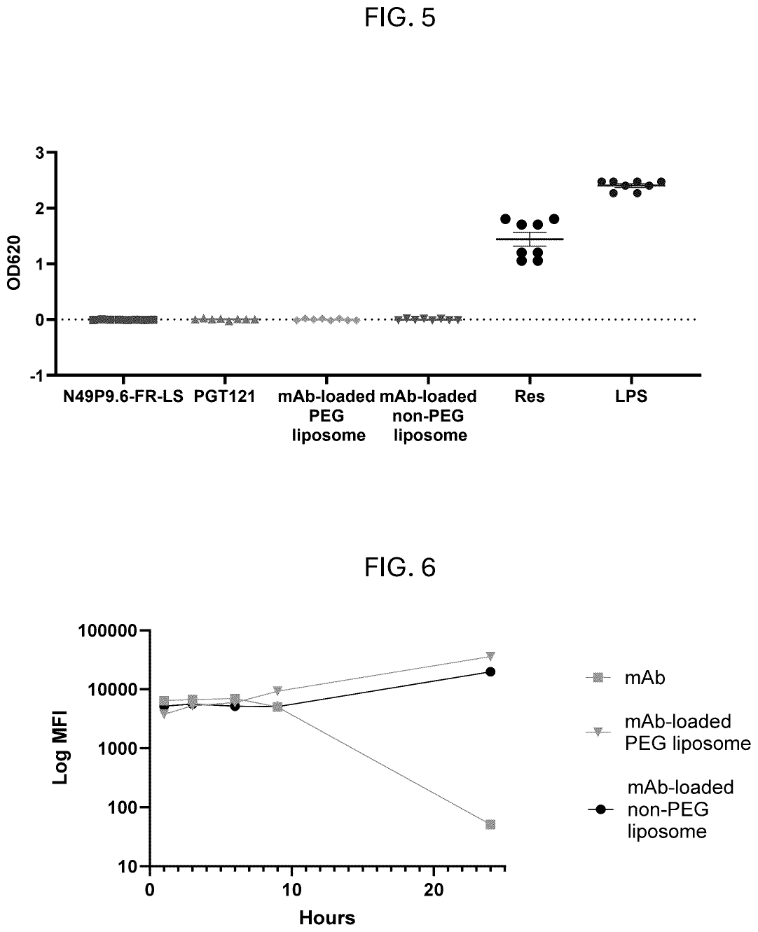

The toxicity of mAb loaded liposomes and controls (free mAb) were assessed against on two cell lines HMEC-1 WT and HMEC-1 FcRn after 24 and 48 h incubation using MTT assay at different concentration of mAb 0.25, 0.5 and 1 g. There was no significant differences in cell viability between the liposomal formulations and the free mAb at different concentrations (FIG. 4A). Furthermore, an apoptosis assay, illustrated in FIG. 4B and conducted via flow cytometry, corroborated the viability test results by demonstrating that both the liposomal and free mAb formulations did not exhibit cytotoxic effects on the cell lines tested. The mAbs and their liposomal formulations did not activate macrophages (FIG. 5).

### Cell Binding and Uptake

The in vitro uptake of the liposome formulations was investigated using flow cytometry at 37° C. for 24 hours. As depicted in FIG. 6, the data indicated that PEG-liposome and non-PEG liposome exhibited equivalent initial uptake into HMEC-1 FcRn cells compared to free mAb. However, for cells treated with the antibody in liposomal form (both PEG and non-PEG), there was a continuous increase in uptake over 24 hours when incubated at 37° C. In contrast, the free antibody (not encapsulated in liposomes) showed a constant uptake for 9 hours, followed by a rapid decline by 24 hours.

### In Vivo Pharmacokinetic Study

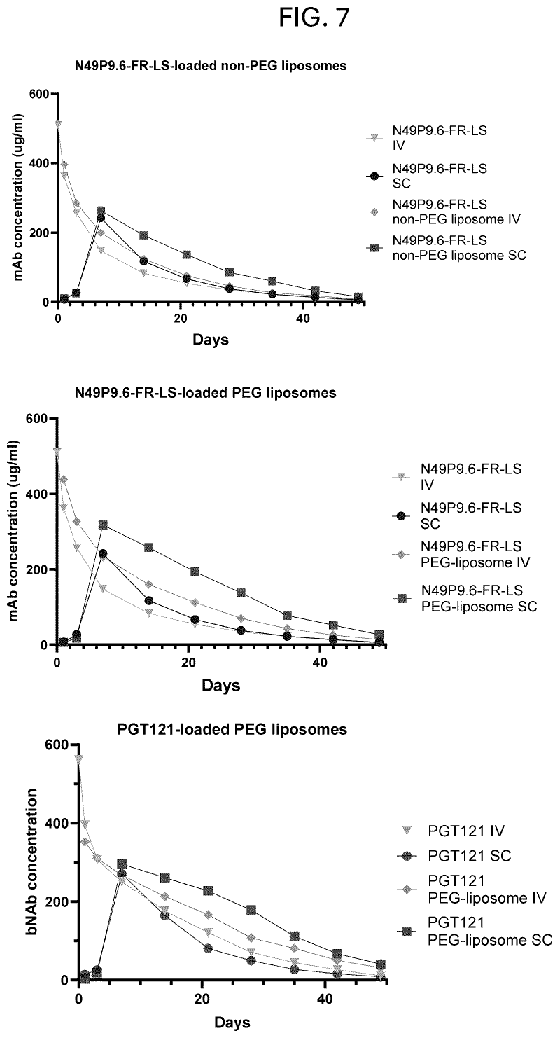

We evaluated the drug release kinetics in vivo for the liposomal formulations and conducted initial pharmacokinetic studies with different formulations, namely PEG-liposome and non-PEGylated liposome loaded with N49P9.6-FR-LS (FIG. 7A-C). Given that the PEG-liposome had better in vitro characteristics, we also studied PEGylated liposomes loaded with another anti-HIV mAb, PGT121.

Each mAb-loaded liposome formulation was injected SC or IV into genetically engineered mouse models established to express human FcRn (FcRn mice). Plasma samples were then collected on days 1, 3, and 7 days, and then weekly, and analyzed for drug concentration. When comparing SC to IV injections for free mAb, a one week delay is seen in reaching the Cmax in the SC group (FIG. 7A-C). The bioavailability of the SC were lower than the IV in comparing free mAb injection, with N49P9.6-FR-LS being 15% lower and PGT121 37% lower in SC vs IV injections (Table 3). Liposomal formulations of the same mAbs again demonstrated the one week delay in reaching Cmax in the SC vs IV groups (FIG. 7A-C).

Both liposomal formulations (PEG and non-PEG) of N49P9.6-FR-LS, when injected SC, improved all measured parameters. The results demonstrated that the liposomal SC administration of the mAb had higher half-life, Cmax, AUC, and MRT and higher CL values compared to the free mAb formulations via the same route (Table 6). The PEGylated formulations generally had higher half-life, Cmax, AUC, and MRT and higher CL values compared to their non-PEGylated formulations of the same injection type, and this was significant when comparing the AUC of the SC PEG formulation (6939) to SC non-PEG formulation (5153) (P<0.05) (Table 6).

The bioavailability of the liposomal formulations were roughly equivalent whether injected SC or IV, though SC liposomal injection had higher bioavailability than IV liposomal injection. When comparing the gold standard (IV injection of free mAb) to SC injection of PEGylated liposomal formulation, the SC liposomal had higher bioavailability than the IV injections, with the PGT121-loaded PEG liposomes having 26% higher bioavailability, and the N49P9.6-FR-LS-loaded PEG liposomes having 80% higher bioavailability (P<0.05 for each, respectively) (Table 6).

Liposomal formulation increased the t1i2 of the mAbs whether injected SC or IV when compared to the injection of the free mAb by SC or IV. This was statistically significant (P<0.05) when PGT121-loaded PEG liposomes were injected SC or IV vs. free PGT121 injected SC, and also when N49P9.6-FR-LS-loaded liposomes were injected SC vs. free N49P9.6-FR-LS injected SC (Table 6).

TABLE 6
Pharmacokinetic parameters of mAb-loaded liposomes.

| | | | | | | | | CL |
|---|---|---|---|---|---|---|---|---|
| | | | | | | Δ t_{1/2} | | ((μg)/ |
| | | Cmax | AUC0-t | Δ AUC | t_{1/2} | (free | MRT | (μg/ |
| Formulations | Injection | (μg/ml) | (μg/ml*d) | (free IV) | (d) | IV) | (d) | ml)/d) |
| Free N49P9.6-FR-LS | IV | 510.25 | 3850 | X | 9 | X | 11.45 | 0.25 |
| Free N49P9.6-FR-LS | SC | 242.25 | 3260 | −15% | 7 | −22% | 16.12 | 0.29 |
| N49P9.6-FR-LS non- | IV | 396.75 | 4864** | +26% | 9.45 | +5% | 12.88 | 0.22 |
| PEG liposome | | | | | | | | |
| N49P9.6-FR-LS non- | SC | 263.75 | 5153** | +34% | 10 | +11% | 19.37 | 0.18 |
| PEG liposome | | | | | | | | |
| N49P9.6-FR-LS | IV | 438.25 | 6106*, ** | +59% | 10 | +11% | 14.45 | 0.158 |
| PEG liposome | | | | | | | | |
| N49P9.6-FR-LS | SC | 317.72 | 6939*, ** | +80% | 12** | +33% | 21.14 | 0.13 |
| PEG liposome | | | | | | | | |
| Free PGT121 | IV | 561.5 | 6351 | X | 10 | X | 14 | 0.15 |
| Free PGT121 | SC | 270.5 | 4015 | −37% | 8 | −20% | 16 | 0.24 |
| PGT121 PEG liposome | IV | 352.25 | 7536*, ** | +19% | 12 | +20% | 18.6 | 0.12 |
| PGT121 PEG liposome | SC | 295.5 | 7991*, ** | +26% | 14.5** | +45% | 24 | 0.11 |

*P < .05 when compared to corresponding free IV mAb injection
**P < .05 when compared to corresponding free SC mAb injection

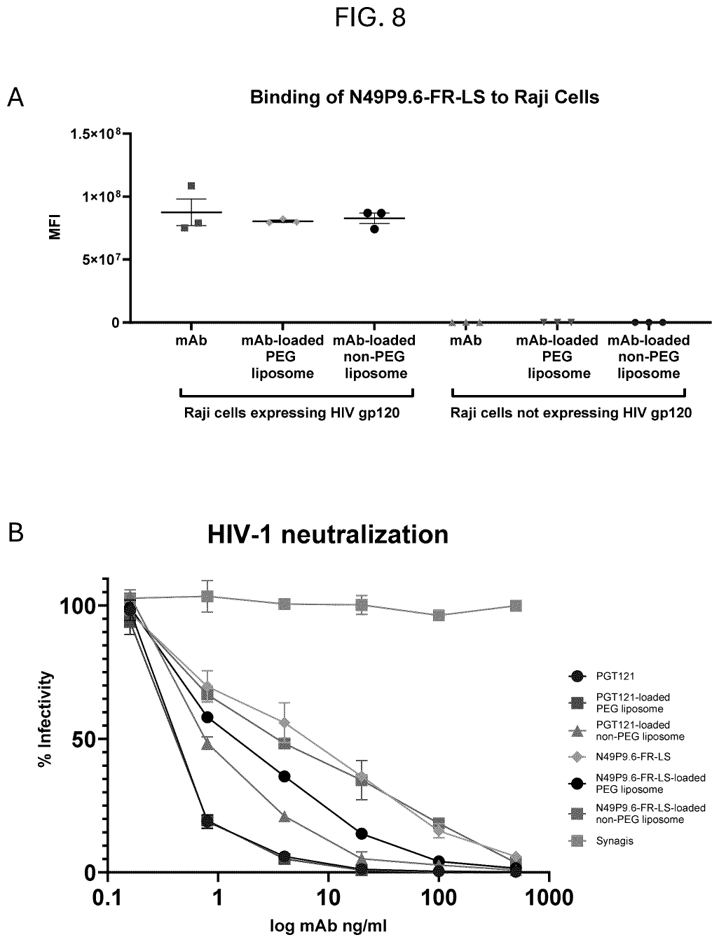

Antibody structure and function was assessed in 3 ways. Mouse plasma samples were measured for concentration of intact, properly folded antibody was measured in plasma (FIG. 7). In addition, ability of mouse plasma to bind HIV-1 gp120 (FIG. 8A) and neutralize HIV-1 Bal pseudovirus (FIG. 8B) was analyzed, both of which were preserved.

### Methods and Materials

### Materials

Hydrogenated soy phosphatidylcholine (HSPC), cholesterol (Chol), and Distearoyl-Glycero-3-Phosphoethanolamine-methoxy polyethylene glycol 2000 (DSPE-mPEG2000), as well as 1,2-distearoyl-sn-glycero-3-phospho-(1′-rac-glycerol) (sodium salt), were sourced from Avanti Polar Lipid (Alabaster, USA). Dioctadecyl Tetramethyl indocarbocyanine perchlorate (DiI) dye, Dimethyl sulfoxide (DMSO), ethanol, and chloroform were acquired from Sigma-Aldrich (St. Louis, US). All additional chemicals and solvents used were of analytical grade. Dulbecco's modified Eagle's medium (DMEM) RPMI-1640 culture media, phosphate-buffered saline (PBS), and fetal bovine serum were purchased from Gibco (Thermo Fisher Scientific, USA).

### Maintenance of Cell Lines

Human microvascular endothelial cell lines (HMEC-1 wild type and FcRn) were obtained from American Type Culture Collection (ATCC), Manassas, VA, USA) and cultured in MCDB131 media (without L-Glutamine; Life Technologies) supplemented with 10 ng/mL Epidermal Growth Factor (EGF; Thermofisher) 1 μg/mL Hydrocortisone (Sigma), 10 mM Glutamine (ATCC 30-2214) and 10% Fetal Bovine Serum (FBS; ATCC). All cells were maintained in the 5% CO2 atmosphere in a humidified incubator at 37° C. The RAW-BLUE™ mouse macrophage reporter cell line (InvivoGen, San Diego, California, USA) was cultured in DMEM medium supplemented with 10% fetal bovine serum (FBS), 100 mg/mL NORMOCIN™, 2 mM glutamine, and 200 g/mL ZEOCIN™. HMEC-1 cells, genetically modified to express human FCGRT, were generously provided by Dr. Wayne L. Lencer. Raji-Env cells were a kind gift from Dr. Olivier Schwartz.

### Production and Purification of Monoclonal Antibody

The mAbs used in this study are N49P9.6-FR-LS and PGT121. N49P9.6-FR-LS and PGT121 are both being evaluated for HIV treatment and prevention. PGT121 has been in Phase 1 human trials while N49P9.6-FR-LS is in the preclinical development phase (Stephenson et al. Nat Med. 2021; 27(10):1718-24; Sobieszczyk M E et al., The lancet HIV, (2023), 10:e653-e62). Both are broadly neutralizing antibodies against HIV, that is, they have the ability to potently neutralize a wide range of HIV strains from around the world. The mAbs were produced by Atum (Newark, CA). Briefly, protein expression vectors were stably transfected into CHO-K1 GS knock-out and expressed in fed-batch using standard ATUM methods, with Protein A resin was used to capture proteins. Resin was washed, protein was eluted and immediately neutralized. Proteins were buffer exchanged into PBS, pH 7.0. Proteins were quantified by OD280, quantity and concentration was determined using calculated extinction coefficient. SDS-PAGE (Biorad criterion Tris/Glycine/SDS, 4-20%) was used to determine purity and approximate molecular mass, and negative endotoxin verified by Charles River Endotoxin Kit. Lack of aggregation was determined by HPLC-SEC with a column of 300A pore size, detection at 280 nm and PBS as the running buffer. Proteins were frozen after filter sterilization.

### Liposome Preparation and mAb Loading

Two different liposomal formulations were generated using the remote loading method with varying molar ratios of phospholipids HSPC, DSPE-mPEG2000, Chol, and DPPG. The lipids dissolved in chloroform were initially mixed within a round-bottom flask according to the molar percentages outlined in Table 4. The solution underwent vacuum evaporation using a rotary evaporator (RE-200A, USA LAB, USA) and was then lyophilized overnight with a lyophilizer (FD 3.0, ATR, USA) to eliminate residual traces of the solvent. The resulting lipid film was then hydrated with mAbs dissolved in phosphate-buffered saline (PBS). The mixture was vigorously agitated using a vortex shaker to ensure thorough dispersion of the lipid blend in the buffer, forming large multilamellar vesicles (MLVs). These MLVs were then subjected to sonication in a bath sonicator (Bandelin, Germany) at 55° C. for 5 minutes. The mixture was then placed in a rotator overnight at room temperature, followed by 4 hours of incubation at 4° C. Afterward, liposomes were extruded through 400, 200, 100, and 50 polycarbonate filters (Whatman, Maidstone, UK) using a jacketed extruder (Genizer, Irvine, CA) to produce small unilamellar vesicles (SUVs). Each formulation underwent 11 cycles of extrusion through each filter. The temperature for preparing each liposomal formulation was determined based on the phospholipids' phase transition temperature (Tm). To remove any unencapsulated mAbs, dialysis was conducted using 300 kDa molecular weight cut-off (MWCO) dialysis membranes against PBS four times at a ratio of 1 to 100 during 24 hours at 25° C. Finally, all the prepared liposomal formulations underwent sterilization through filtration using a 0.22 m syringe filter (Hoseini et al., Scientific Reports, (2023), 13:18012).

### Nanoliposome Characterization

The Dynamic Light Scattering (DLS) equipment (Nano-ZS; Malvern, UK) was utilized to analyze the particle size, Polydispersity Index (PDI), and surface charges of the formulations (Stetefeld et al., Biophysical reviews, (2016), 8:409-27). Transmission electron microscopy (TEM) with negative staining was employed to investigate the morphological characteristics of the formulations (Scarff et al., JoVE (Journal of Visualized Experiments), (2018), (132):e57199). For the sample preparation for TEM imaging, the original liposome solution was diluted at a ratio of 1:40 (liposome to PBS), and 20 L of this diluted sample was deposited onto a carbon-coated copper grid. After 1-minute, excess liposomes were eliminated using filter paper. Then, 20 L of filtered uranyl acetate (2% w/v) was applied to the grid. After drying, the samples were examined and photographed using a LEO 912 TEM at an accelerating voltage of 80 kV.

The quantities of antibodies entrapped within liposomes, presented as encapsulation efficiency percentages, were determined using a commercial BCA Protein Assay Kit with a detection range of 20 to 2000 g/mL (Thermo Scientific, USA) (Agallou et al., Journal of Controlled Release, (2023), 356:386-401). Before conducting the BCA analysis on liposomes, liposome preparations were treated with TCA reagent (72% (w/v) trichloroacetic acid in ultrapure water) and Sodium deoxycholate reagent (0.15% (w/v) sodium deoxycholate in ultrapure water), as recommended by manufacturer. We used the following formula for calculating the percentage of encapsulated antibodies:

% antibody entrapped=(Protein content after liposome purification through dialysis/Protein content before liposome purification through dialysis)×100.

In Vitro Release of mAb from Liposomal Formulation

The mAb release profile and leakage stability of the liposomal formulations were evaluated under in vitro conditions in 9 ml of 50% sterile human plasma in 5% sterilized dextrose, along with 1 ml of liposomes containing 1.7 and 1.6 mg of mAb in two liposomal formulations at pH 7, maintained at 37° C. for 42 days in sterile conditions. The mAb-loaded liposomes were placed in a dialysis cassette (300 kDa MWCO), with the dialysis cassette immersed in a beaker containing 100 mL of release medium and incubated at 37° C., stirring at 100 rpm under sterile conditions. At specified time intervals (1, 3, 6, 12, and 24 hours, 3d, 7d, and then every week up to day 42), 500 L of the incubated medium was collected under sterile conditions and centrifuged for 20 min at 4000 g using Ultra-4 centrifugal filter units (300-kDa MWCO, Millipore, USA). Subsequently, 500 L of fresh medium was replenished in the beaker. The concentration of mAb released from the liposomal formulations was determined using an indirect ELISA that measures intact human IgG.

### Stability of Liposomes

The stability of mAb-loaded liposomes was evaluated under controlled conditions, with the samples stored in a sealed and dark environment at both 4° C. and 25° C. (23). For this aim, samples from the liposomal formulations were regularly collected at various time intervals (0, 8 and 16 weeks). Dynamic light scattering (DLS) determines the particle size and surface charge. The physical stability of these samples was examined in terms of particle size, encapsulation efficacy, visual appearance, mAb leakage, and the propensity to form sedimentation. The leakage of mAb from each liposome was quantified as the percentage of encapsulation efficiency (EE %) after two and four months relative to the EE % measured at the initial time point (time 0). EE measured as above.

### Cytotoxicity Assay

The cytotoxic effect of mAb-loaded liposomes was assessed using an MTT assay (Fan D et al., Signal Transduction and Targeted Therapy, (2023), 8:293). In this experiment, 5×10³ cells per well of HMEC-1 cells were seeded in 96-well plates. After overnight incubation at 37° C., various concentrations of mAb-loaded liposomes were applied to the cells. Subsequently, the cells were incubated at 37° C. for 48 and 72 hours. Following the incubation period, the treatments were removed, and the wells were washed with PBS before being treated with MTT solution for 240 minutes in the dark at 37° C. After incubation, the media was discarded and replaced with 200 L of DMSO. The absorbance of each well was measured at a wavelength of 570 nm using an ELISA reader. All treatments were conducted in triplicate. The IC50 values were determined using CalcuSyn version 2 software (BIOSOFT, UK). The viability of cells was calculated by the following equation:

$$\% Viability = \frac{Mean OD Sample - Mean OD blank}{Mean OD Untreated Cell Control - Mean OD blank} \times 100$$

### In Vitro Cell Binding and Uptake

The liposome uptake assay was performed using flow cytometry on HMEC-1 and HMEC-1 FcRn cells, which were seeded at 5×10⁵ cells per well in a 12-well plate. These cells were exposed to antibodies and liposomes for 1, 3, 6, 9, and 24 hours. Following treatment, the cells were trypsinized, washed twice with washing buffer, and fixed with PFA for 10 minutes at room temperature. After fixation, the cells were permeabilized for 30 minutes and stained with Goat F(ab)2 anti-human IgG (SouthemBiotech) for 30 minutes at 4° C. Flow cytometry analysis was then conducted. The results indicated uptake of both liposomes and antibodies by the cells. Notably, PEGylated liposomes containing antibodies showed prolonged retention within the cells compared to antibodies alone and non-PEGylated liposomes. Finally, the cells were resuspended in 200 L of staining buffer for flow cytometry analysis (BD Canto II, BD Biosciences, San Jose, USA).³⁰

The liposome uptake assay was conducted via flow cytometry using HMEC-1 and HMEC-1 FcRn cells seeded at 5×10⁵ cells per well in a 12-well plate. A 0.2 M ratio of the DiI fluorescent dye, previously utilized in liposome preparation, was applied. Subsequently, the cells were exposed to the infrared fluorescent lipophilic dye, 1,1′-dioctadecyl-3,3,3′,3′-tetramethylindotricarbocyanine iodide ((DiR) Thermo Fisher, Waltham, MA, USA), to label liposomes with DiR at 4° C. and 37° C. for varying durations of 1, 2, 3, 6, 12, and 24 hours (Teymouri et al., International journal of pharmaceutics, (2016), 511:236-44).

After incubation, cells were washed with cold PBS, trypsinized with 0.1 mL of Trypsin-EDTA solution, and transferred to 2 mL tubes. The tubes were then centrifuged at 1500 g for 8 minutes, and staining buffer (2% FBS in PBS) was used to wash the cells and remove remaining supernatant. Finally, cells were resuspended in 200 L of staining buffer for flow cytometry analysis (BD Canto II, BD Biosciences, San Jose, USA) (26).

### Neutralization Assay

In order to test the preservation of functional activity of mAbs after liposomal encapsulation, HIV-1 neutralization testing was performed using a luciferase-based assay in TZM.b1 cells, as previously described (Sajadi et al., Cell, (2018), 173:1783-95 e14). This assay measures the reduction in luciferase expression following a single round of virus infection. Stocks of Env-pseudotyped viruses were prepared by transfection of 293T cells, as previously described (Sajadi et al., Cell, (2018), 173:1783-95 e14). Serum samples were tested against HIV BaL.26 pseudovirus. Three-fold serial dilutions of samples were tested in duplicate (96-well flat bottom plate) in 10% D-MEM growth medium (100 uL/well). 200 TCID50 of pseudovirus was added to each well in a volume of 50 uL, and the plates were incubated for 1 hr at 37° C. TZM.b1 cells were added (1×104/well in 100 uL volume) in 10% D-MEM growth medium containing DEAE-Dextran (Sigma, St. Louis, MO) at a final 11 μg/mL concentration. The final volume for each well was 250 ul. Assay controls included replicate wells of TZM.b1 cells alone (cell control), TZM.b1 cells with virus (virus control), and MuLV control. Following a 48-hour incubation at 37° C., 150 μL of assay medium was removed from each well, and 100 μL of Bright-Glo luciferase reagent (Promega, Madison, WI) was added. The cells were allowed to lyse for 2 min, then 150 μL of the cell lysate was transferred to a 96-well black solid plate, and luminescence was measured using a Victor 3 luminometer (Perkin Elmer, Waltham, MA).

### Caspase-3 Apoptosis Assay Followed by Flow Cytometry

The potential cytotoxicity of liposomes and on HMEC1 cells was determined by flow cytometry using a caspase-3 apoptosis assay. Briefly, cells were trypsinized after treatment with the liposomes or heat treated for 7 min at 55° C. (apoptosis/dead induction control), stained with 1 μl of the cell-permeable caspase-3 inhibitor DEVD-FMK conjugated to sulfo-rhodamine (Red-DEVD-FMK) that binds irreversibly to activated caspase-3 (CaspGLOW Red Active Caspase-3 Staining kit, BioVision), incubated at 37° C. for 45 min, washed three times and analyzed using a FACSCanto II (BD Biosciences) instrument and FlowJo v.8.5 (Becton, Dickinson and Company).

### Pharmacokinetics & Pharmacodynamics Study

All in vivo studies were approved by the Institutional Animal Care and Use Committee of the State University of Maryland at Baltimore (IACUC #0120004). Groups of 4-5 humanized FcRn male and female mice (˜6 weeks old, 19-26 g) were used for pharmacokinetic study, which studied IV and SC injections of the mAbs with and without liposomal encapsulation. 30 mg/kg of N49P9.6-FR-LS (with and without liposomal encapsulation) was injected either IV (retroorbital) or SC (flank area). Blood samples were collected before injection and on days 1, 3, 7, and then weekly using microcapillary blood collection EDTA tubes (Ram Scientific, Germany). Afterwards, the blood was centrifuged at 2000 g for 10 min at 4° C. Plasma was collected, and the concentration of IgG in each mouse was measured using indirect Elisa.

The methodological approach in the indirect ELISA protocol utilized for quantifying concentrations of IgG encompassed the following steps: a) Coating the Immunol plate (Thermo IMMULON 2HB, Cat #3455) with 50 L per well of 1 g/mL unlabeled anti-human kappa and anti-human lambda in 1×TBS and incubated overnight at 4° C. (b) blocking the nonspecific binding sites on the plate by Blotto+NP40 (c) adding samples, and standards to the plate, (d) adding detection antibody to the plate, and (e) adding the substrate. Between each step, plates were washed four times with 1% phosphate buffer saline (PBS)-Tween (0.05% Tween-20 in 1×PBS, no pH adjustment) except for the secondary antibody adding step, which requires six times washing. Finally, add 50 L 1× blue phosphate solution to each well to stop the reaction. The change in absorbance was measured over time (dA/dt) at 620 nm using SpectraMax Paradigm (Molecular Devices, Sunnyvale, CA). All standard curves were fitted using 4-parameter logistic equations. Pharmacokinetic parameters included the maximum plasma concentration (Cmax), the area under the concentration-time curve (AUC), the half-life of elimination (T½), the average time a particle remains in the plasma compartment, and the mean residence time (MRT).

### Statistical Analysis

Data were statistically analyzed using GraphPad Prism (version 10, San Diego, US). Kaplan-Meier survival analysis was used to compare different groups. Pharmacokinetic parameters were analyzed using a PKSolver derived from non-compartmental analysis (Zhang et al., Comput Methods Programs Biomed, (2010), 99:306-14). Flow cytometry data analysis was performed using FlowJo Software version 10 (FlowJo, Ashland, US).

### Conclusion

SC formulations of mAbs constitute a large fraction of the ever-growing field of immuno-therapeutics. Compared to IV administration, SC can dramatically decrease the cost of delivery because injection does not necessarily require skilled nursing, and it can be done outside a healthcare facility, in the privacy of the home, by the patient themselves. However, compared to IV administration, there can be several significant disadvantages. Because of transit through the lymph system, SC injections tend to have less bioavailability and at times a lower half-life. After SC injection, large amounts of free antibodies can degrade at the injection site, be eliminated through the lymph transit, or, even worse, lead to immunogenicity which could lead to permanent failure of the mAb.

We hypothesized that liposomal formulations of mAbs could protect the mAb in the SC space, and through its lymph transit, thereby leading to improved PK. Although previous studies showed that mAbs encased in liposomes retained their native structure, we are not aware of any studies that sought to demonstrate their potential for improving PK (Nikpoor A R et al., International journal of pharmaceutics, (2015), 495:162-70).

The most striking findings in this study were that liposomal formulations of mAbs had improved PK over free mAbs, regardless of injection route. The liposomal formulation of mAbs, when injected SC, had about a week lag before reaching Cmax and was, in this respect, similar to the free antibody injected SC. This indicates that the liposomes underwent lymph transit in bulk, whereby they were deposited into the bloodstream. However, the improved PK indicate that the liposomes protected the mAbs from degradation in the SC space and/or through lymph transport. That there were no significant PK differences between liposomal formulations that were administered IV or SC (other than the Cmax lag) is a further indication of the protection of the SC-injected mAbs by liposomal encapsulation.

Importantly, bioavailability of SC liposomal formulation not only surpassed that of SC free mAb, but was better than the gold standard IV free mAb injection. This was seen in all three mAb-liposome combinations that were tested in vivo, and improvement in bioavailability were 26-80% over IV free mAb injection. The half-life of the mAbs improved as well, but this last improvement was more modest, with only an improvement in several days. However, given that free SC injection of mAbs can be associated with lower half-lives and greater immunogenicity, the preservation of the mAbs through SC transit and their longer half-lives in liposomal formulations could be used as a strategy to both preserve functional activity and extend injection intervals.

The PK improvements were gained with liposomal formulations that were designed for specific sizes and charges, which influences their clearance from the bloodstream and availability in plasma. Overall, smaller liposomes tend to circulate longer, enhancing bioavailability, while larger ones may be cleared more quickly by the mononuclear phagocyte system (Jain R K et al., Nature reviews Clinical oncology, (2010), 7:653-64; Dreffier C et al., International journal of pharmaceutics, (2003), 254:43-7). We chose to study a liposome size (<100 nM) that would facilitate transit through the lymph system (33). The DLS measurements and TEM imaging have shown that liposome sizes and (PDIs) were less than 100 nm and 0.18, respectively.

Surface charge strongly affects the stability, pharmacokinetics properties, and affinity of the liposomes with entrapped drugs, and the liposomes in this study were designed to carry a negative charge. Charged liposomes are less prone to aggregate; negatively charged liposomes improve stability and aggregation, and are less prone to phagocytosis than positively charged ones (Gbian D L et al., Biomedicines, (2022), 10:2137; Dave V et al., Nano-Structures & Nano-Objects, (2019), 18:100288). Zeta potential is a crucial factor used to approximate liposomes' surface charge. The PEGylated and non-PEGylated liposomes' zeta potentials (mV mean) were around −12 and −40 mV, respectively. In non-PEGylated liposomal formulations, DSPG is a key ingredient in generating a negative surface charge, facilitating the creation of anionic liposomes with favorable physicochemical properties (Tsermentseli S K et al., Fluids, (2018), 3:36). The procedure of adding polyethylene glycol (PEG) to liposomes (PEGylation) can provide a ‘stealth’ characteristic by giving the liposome a hydrophilic polymer layer. This hydrophilic effect helps avoid detection by the mononuclear phagocyte system, further postponing clearance from the bloodstream and extending the drug's half-life, declining immunogenicity, and antigenicity (Bulbake U et al., Pharmaceutics, (2017), 9:12). PEGylated liposomes with relatively smaller surface charges are more appropriate for SC injection than non-PEGylated liposomes (Harrington K J et al., Clinical cancer research, (2000), 6:2528-37; Allen T et al., Biochimica et Biophysica Acta (BBA)-Biomembranes, (1993), 1150:9-16). Pharmacokinetic investigations in this study demonstrated that both PEGylated and non-PEGylated liposomal encapsulation of mAb improved half-life, AUC, and MRT while reducing clearance and enhancing circulation time and efficacy via both SC and IV routes. PEGylated liposomes provided better half-life results due to the hydrophilic polymer coating, which reduced recognition and clearance by the mononuclear phagocyte system, leading to prolonged circulation and improved stability.

Maintaining liposomes' physical and chemical stabilities is essential for their shelf-life and efficacy. Physical instabilities, like drug leakage, vesicle fusion, and chemical instabilities from hydrolysis or oxidation, can reduce in vivo efficacy and the drug's therapeutic potential (Zhou W et al., Colloids and surfaces B: Biointerfaces, (2014), 117:330-7). Our findings showed that both PEGylated and non-PEGylated liposomes were relatively stable at up to 4 months, especially at 4° C. The negative charges provides substantial protection against alterations (Karimi et al., International journal of pharmaceutics, (2020), 580:119211; Chen X et al., Molecules, (2015), 20:14293-311). Additionally, liposomes with lipids of high phase transition temperature (Tm) (HSPC=58° C. and DSPG=55° C.) and the inclusion of cholesterol showed increased stability, rigidity, and reduced drug leakage, emphasizing the importance of lipid composition in liposomal formulation stability (Briuglia M-L et al., Drug Delivery and Translational Research, (2015), 5:231-42, Li J et al., Asian Journal of Pharmaceutical Sciences, (2015), 10:81-98). DSPG addition has been shown to promote better drug encapsulation and optimize particle size distribution (Tsermentseli S K et al., Fluids, (2018), 3:36). Moreover, when DSPG is included in PEGylated formulations loaded with mAb, the resultant liposomes exhibit exemplary stability and drug release profiles.

It is important to note that although the liposomes are extremely stable in vitro (on the order of months) and seem to be stable during lymph node transit (for a week), they seem to open up rapidly once in the blood. This is likely due to the release of the mAbs from encapsulation over several days in the blood, and the similar half-lives of the IV and SC liposome formulations attest to this.

This study demonstrates the potential of liposomal formulations to enhance the pharmacokinetic profile of mAbs, mainly when administered SC. While SC administration offers significant advantages in terms of cost and convenience, it often comes with drawbacks, such as reduced bioavailability and shorter half-life compared to IV administration. The introduction of liposomal encapsulation represents a promising strategy to address these limitations. SC-administered liposomal formulations of mAbs demonstrated superior pharmacokinetic parameters compared to free mAbs, even when the latter is administered IV. Notably, liposome encapsulation protects mAbs from degradation in the SC space and during lymph transit, leading to improved bioavailability and extended half-life. Both PEGylated and non-PEGylated liposomal formulations show significant enhancements in half-life, area under the curve (AUC), mean residence time (MRT), and reduced clearance, prolonging circulation time and enhancing efficacy.

### Example 2. Development and Testing of Liposomal Pharmaceutical Formulations of Antibodies for Subcutaneous Delivery

The major limitation hindering the use of monoclonal antibodies (mAbs) in general, and anti-HIV broadly neutralizing antibodies (bNAbs) in specific is related to the pharmacokinetics (PK) of injected antibody. Native IgG has a half-life of 20 days in adults. In order to be used for HIV prevention, the half-life of bNAbs should be on the order of months (preferably ≥9 months for practical use). While a therapeutic use such as in a cure strategy may not need extended exposure, its use as an ARV substitute would need an extended half-life also. Current strategies to increase the half-life of antibodies relies on strategies to increase Fc binding to FcRn, preventing endosomal degradation of IgG. These modification include the “LS” variant, which will contains 2 point mutations at positions M428L and N434S in the Fc domain, as well as the “YTE” variant, which contains 3 points mutation at positions M252Y, S254T, and T256E in the Fc domain. However, there appears to be a limit to the half-life extension targeting FcRn, with the maximal half-life of Fc-modified IgG1 in adult humans being around 3-4 months. HIV mAbs tested in clinical trials thus far have had shorter half-lives, even in healthy adults, when there would not be antigen-antibody complexes leading to faster clearance. In humans, the half-lives for VRC01-LS and VRC07-523-LS have been determined (71 and 38 days, respectively) A trial of 3BNC117-LS has been completed, but the results are not yet reported. The relatively short half-life of VRC07-523 LS (only 38 days despite carrying the “LS” mutation) will hinder efficacy, consistent with the AMP trial models for thresholds of effective plasma titers.

Beyond the half-life, the delivery of mAbs and bNAbs is also an issue. The AMP trial injected VRCO1 by the intravenous (IV) route, which requires placement of an IV by a skilled nurse, hanging an IV bag, and monitoring the patient during the actual infusion. All of these steps increase the cost and ability to deliver the intervention. By contrast, subcutaneous (SC) injections are relatively quick and inexpensive, and ideal for resource-poor settings, and can even be administered by the patient at home. However, SC administration of antibody is hampered by proteolytic degradation at the site of injections and via clearance during transit from skin through lymph nodes. Compared to IV, with SC injection the bioavailability in humans appears to be lower, with decreases in half-life, and increased immunogenicity and anti-drug antibody (ADA).

Thus, we devised a strategy using liposomes to shepherd SC injected antibodies intact until delivery to the blood. Liposomes are phospholipid vesicles consisting of one or more concentric bilayers composed of phospholipids. Our deign strategy centered on selecting liposomes that would have extended half-lives (pegylated forms, and those with lipid bilayers instead of monolayers).

Liposomes preparations using various ratios of HSPC, DSPG, DSPE, and cholesterol (see Table 7 below for ratios and full chemical composition) were dried on a rotary evaporator. All these phospholipids are FDA approved and obtained from Avanti Polar Lipids, Inc. (Alabaster, Alabama). The lipid film was hydrated with 1 mg/ml of bNAb dissolved in 10 mM pH 7.4 PBS under argon gas and swirling at 54° C. in a water bath for 30 min, followed by overnight incubation at 4° C. Liposomes were then extruded and dialyzed using a 300-kDa molecular weight cut-off (MWCO) filter, then through a 0.45 m syringe filter. After preparation of liposomes, the size and polydispersity index was determined with DLS (Zetasizer, Malvern), encapsulating efficiency measured with HPLC (Infinity 11 1260 LC, Agilent), and release profile measured (half-life at 4° C. and 37° C. in sterile human plasma in 5% sterilized dextrose) (Table 7).

TABLE 7
Characteristics of liposomes

| | non-PEG | PEG 5 |
|---|---|---|
| Lipid | HSPC/DSPG/ | HSPC/DSPG/Chol/ |
| composition | Chol | DSPE-mPEG2000 |
| Lipid molar ratio | 55/5/40 | 50/5/40/5 |
| Size (nm) | 95.7 ± 8.5 | 89.8 ± 7.9 |
| Polydispersity | 0.21 ± 0.02 | 0.33 ± 0.05 |
| Encapsulation | 58% | 61% |
| efficiency | | |
| In vitro half-life | 4.1 months | 6.3 months |
| 4° C. | | |
| In vitro half-life | 2.7 months | 3.5 months |
| 37° C. | | |
| (human plasma) | | |

Values are presented from triplicate measurements of each formulation. The data are represented as mean ± standard deviation (SD).
PEG: polyethylene glycol;
HSPC: L-α-phosphatidylcholine, hydrogenated (Soy);
DSPG: 1,2-distearoyl-sn-glycero-3-phospho-(1′-rac-glycerol) (sodium salt);
Chol: cholesterol;
DSPE: 1,2-distearoyl-sn-glycero-3-phosphoethanolamino N-[methoxy(polyethylene glycol)-2000] (ammonium salt)

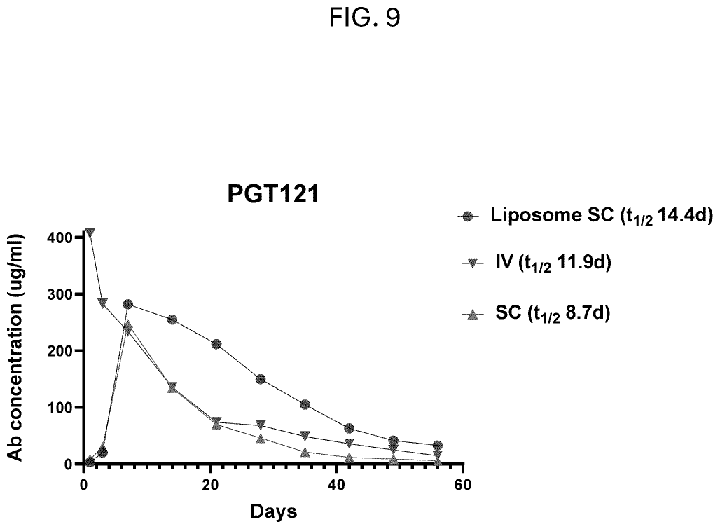

In the next experiment, we tested the in vivo pharmacokinetics of the pegylated liposome formulations. In both antibodies tested, SC injection had decreased half-life compared to IV injection. However, encapsulation in liposomes prior to SC injection protected the antibodies from degradation and led to approximately 100% increase in bioavailability (AUC) and half-life (FIG. 9; Table 8). Serum from the mice demonstrated intact PGT121 and N49P9.6-FR-LS (FIG. 9; in this assay antibody must have both light and heavy chain to be detected), with preserved HIV-1 gp120 binding (Table 8).

TABLE 8
PK by mAb, formulation, dose and route

| | PGT121 | | | N49P9.6-FR-LS | | |
|---|---|---|---|---|---|---|
| Formulation | PBS | PBS | Lip | PBS | PBS | Lip |
| Route | IV | SC | SC | IV | SC | SC |
| Dose (mg/kg) | 40 | 40 | 40 | 40 | 40 | 40 |
| Half-life (days) | 11.9 | 8.7 | 14.4 | 10.0 | 6.8 | 11.5 |
| AUC | 5752 | 3661 | 8539 | 8540 | 6734 | 9108 |
| Cmax (ug/ml) | 303 | 184 | 282 | 458 | 256 | 393 |
| gp120 Elisa | 0.74 | ND | 0.23 | 0.94 | ND | 0.05 |
| EC50 (ug/ml) | | | | | | |

PBS: phosphate buffered saline;
Lip: PEGylated liposome (formulation given in Table 3);
AUC: area under the curve;
Cmax: maximum concentration;
EC50: half maximal effective concentration;
ND = not done

We postulate that the liposome protects IgG from degradation at the injection site and lymph node, also potentially preventing immunogenicity and anti-drug antibodies (ADA) of the bNAb, while further providing steady release (evidenced by increase in half-life compared to IV). We are not aware of published literature evaluating this use for liposomes in terms of protection from degradation, increasing bioavailability, and extension of half-life. When liposomes are injected SC, by 7 days all the liposomes reach the blood, where they begin to release their contents over a number of weeks (FIG. 9). Thus, while liposomes rescue mAbs injected SC, the effect is short-lived (on the order of weeks).

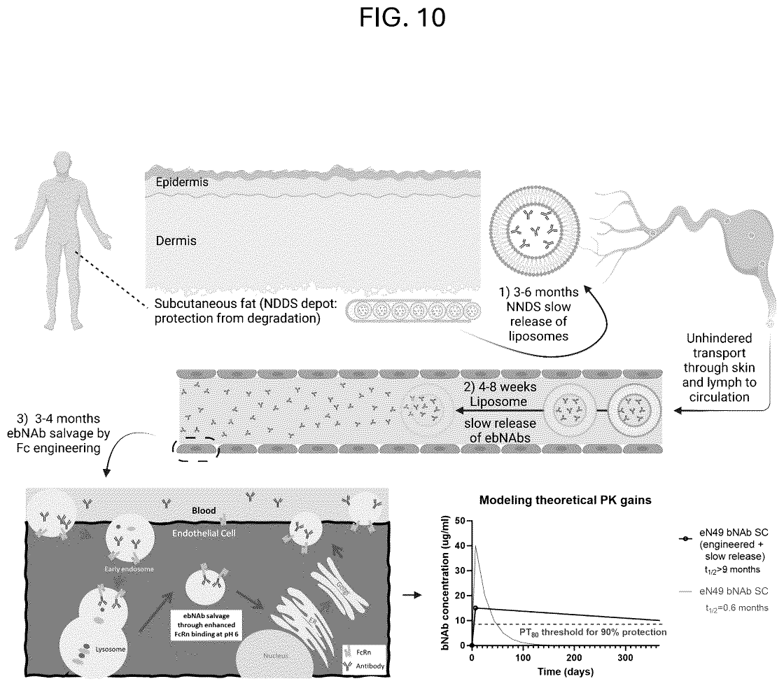

Here we propose to develop an innovative new bi-phasic approach that encapsulates engineered bNAbs (ebNAb) in liposomes, which are themselves placed into silica or hydrogel nano drug delivery system (NDDS) depots, protecting them from degradation (target half-life for liposome itself at this stage equals in vitro: ≥3 months). Phase 1 utilizes silica or hydrogel NDDS depots to slow delivery of the liposomes to the blood over the 3-6 months; Phase 2 is the slow delivery of ebNabs from liposomes into plasma over weeks, where the ebNAbs have a half-lives of 3-4 months. Collectively, this should yield extraordinarily persistent circulating titers (dosing ≥9 months) (see FIG. 10). While the above has designed for delivery of anti-HIV bNAbs, they can be used for delivery of any mAb.

### Example 3. Embedding or Encapsulation of Liposomes in Hydrogels or Microspheres for Subcutaneous Delivery and Sustained Release of Antibodies

Monoclonal antibodies (mAbs) have become essential therapeutics for a wide range of diseases, including cancers, autoimmune conditions, and infectious diseases (Flego et al., BMC medicine, (2013), 11:1-17; Ecker et al., MAbs, Taylor & Francis, (2015), pp. 9-14). In the field of HIV, broadly neutralizing antibodies (bnAbs) have shown exceptional promise in targeting diverse strains of HIV, making them attractive candidates for both therapeutic and prophylactic applications. However, their clinical translation is often constrained by the need for frequent administrations due to their limited half-life and rapid clearance from circulation, especially when administered subcutaneously (SC). (Quinteros et al., Nanostructures for drug delivery, Elsevier, (2017), pp. 807-833). This creates challenges in patient adherence, increases healthcare burden, and underscores the necessity of developing long-acting delivery platforms. Among various administration routes, SC delivery is preferred for its ease of use, reduced need for clinical supervision, and potential for self-administration. Despite these benefits, SC delivery of mAbs is associated with several limitations, including enzymatic degradation at the injection site, low bioavailability due to limited absorption, and increased risk of immunogenicity. Improving SC pharmacokinetics (PK) is critical to reducing dosing frequency and enhancing therapeutic efficacy. Liposomal nanocarriers have been extensively explored for improving the pharmacokinetics of biologics via intravenous (IV) administration. Their ability to encapsulate therapeutic proteins and protect them from degradation renders them a promising strategy for mAb delivery. However, the utility of liposomes in subcutaneous (SC) delivery has been underexplored, largely due to challenges in depot formation and retention at the injection site. In our previous example, we formulated an SC liposomal delivery system that demonstrated improved pharmacokinetics. (Karimi et al., Acta Biomaterialia, (2025), 195:522-535) Building upon these findings, we developed a hybrid delivery system that embeds mAb-loaded liposomes into an injectable hydrogel matrix, enabling sustained release and improved SC bioavailability. Injectable hydrogels have shown significant promise as localized drug depots due to their shear-thinning properties, ease of administration, and compatibility with soft tissue environments. Recent studies have demonstrated that liposome-based supramolecular hydrogels can be engineered to provide orthogonal release mechanisms for protein drugs by tuning liposome surface chemistry and hydrogel mesh properties. (Correa et al., Matter, (2022), 5(6):1816-1838) These liposomal nanocomposite hydrogels (LNHs) offer the dual advantage of preserving protein stability and allowing programmable release through passive diffusion, electrostatic, or affinity-based interactions. In this study, we prepared several formulations in both hydrogel and microsphere forms. We used poly(vinyl alcohol) (PVA) as the hydrogel matrix due to its biocompatibility, injectability, and ability to form physically or chemically crosslinked hydrogels with tunable mechanical strength and diffusion properties. PVA enables depot formation at the SC site and supports sustained diffusion of encapsulated liposomes. (Zahra et al., Polymers, (2023), 15(18):3837) For the microsphere-based formulation, we selected PLGA (75:25) owing to its slower degradation profile compared to 50:50 PLGA, providing prolonged structural integrity and gradual liposome release over time. (Woo et al., Pharmaceutical research, (2001), 18:1600-1606) Building on these concepts, our study introduces a liposome-loaded hydrogel platform modified for SC delivery of mAbs. This delivery system combines the protective and encapsulating capabilities of liposomes with the depot-forming, controlled-release nature of hydrogels. By optimizing both liposome formulation and hydrogel composition, we achieved a SC injectable system that significantly enhances mAb bioavailability and sustains therapeutic levels over extended periods. Here, we present the formulation strategy, characterization, and in vivo evaluation of this delivery platform, with an emphasis on its pharmacokinetic performance and translational relevance.

### In Vitro Release Study

We evaluated the stability and release kinetics of mAb-loaded liposomes embedded within three distinct hydrogel-based delivery systems under in vitro conditions that simulate the physiological environment of subcutaneous (SC) injection. A pH of 7.4 was selected to reflect both the SC space and systemic circulation. The release medium consisted of 50% sterile human plasma in 5% sterilized dextrose, maintained at 37° C. The three hydrogel formulations tested included:
(1) Non-crosslinked PVA hydrogel containing mAb-loaded liposomes
(2) Genipin-crosslinked PVA-gelatin hydrogel containing mAb-loaded liposomes
(3) Gelatin hydrogel embedded with PLGA microspheres encapsulating mAb-loaded liposomes

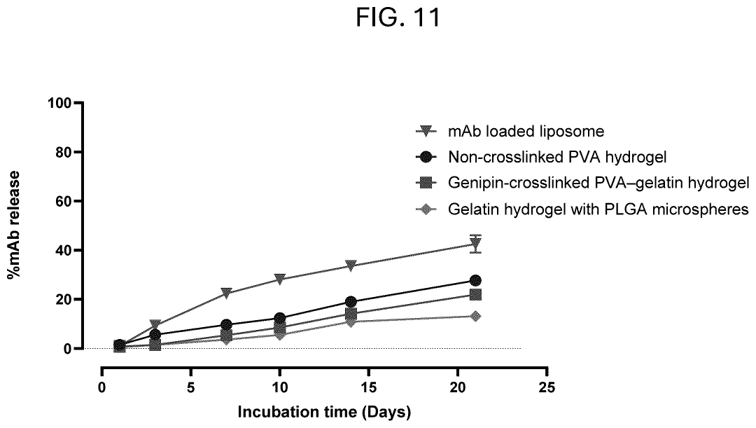

The release profile compares four distinct subcutaneous (SC) formulations of broadly neutralizing antibodies (bNAbs), each exhibiting different release kinetics based on their structural composition (FIG. 11). Overall, the release profiles demonstrated significantly slower in vitro release from all hydrogel-encapsulated liposomal systems compared to free mAbs. The liposome-only formulation released approximately 45% of the encapsulated bNAb over 21 days. Due to the absence of a surrounding matrix, this group showed the fastest release, as there was no structural component to slow down diffusion. The non-crosslinked PVA hydrogel formulation provided slightly better retention, releasing about 27% of the antibody over the same period. While the hydrogel structure offered some diffusion resistance, the lack of crosslinking resulted in a relatively loose network with limited control over release.

A more sustained release was achieved with the genipin-crosslinked PVA-gelatin hydrogel, which released only 21% of the bNAb in 21 days. The genipin crosslinking significantly enhanced the matrix integrity, reducing porosity and limiting the diffusion of the encapsulated liposomes. The most controlled release was observed in the formulation containing PLGA microspheres embedded within a gelatin hydrogel. This system released just 13% of the bNAb over the 21-day period. The combination of a biodegradable microsphere depot and a hydrogel matrix created a dual-barrier system that provided the most effective retention and prolonged release. Overall, these results demonstrate a clear trend: as the structural complexity of the formulation increases, the release rate of bNAb decreases. Incorporating crosslinked networks and biodegradable microspheres significantly improves the sustained release performance, making such hybrid systems ideal for long-acting SC antibody delivery.

### In Vitro Cytotoxicity Assay

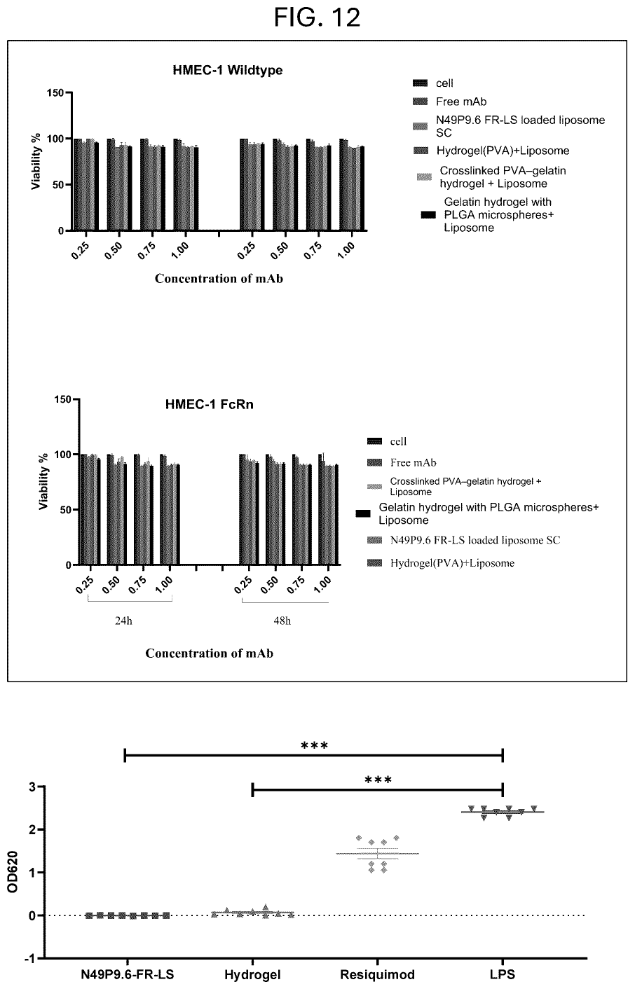

The toxicity of mAb-loaded liposomal hydrogel formulations were assessed in two cell lines, HMEC-1 WT, and HMEC-1 FcRn, after 24 and 48 h incubation using the MTT assay. At both time points, the viability of HMEC-1 WT and HMEC-1 FcRn cells exposed to free mAb and mAb-loaded liposomes (0.25, 0.5, and 1 g/mL) remained high across all treatment groups. As shown in FIG. 12A, no significant reduction in cell viability was observed in either cell line, regardless of FcRn expression, when compared to untreated controls. Additionally, apoptosis analysis via caspase-3 staining (FIG. 12B) confirmed minimal apoptotic activity in both wild-type and FcRn-deficient HMEC-1 cells. These findings indicate that neither the liposomal formulation nor the Hydrogel formulations induced cytotoxic or pro-apoptotic effects after 24-48 hours of treatment.

### Pharmacokinetics & Pharmacodynamics Study

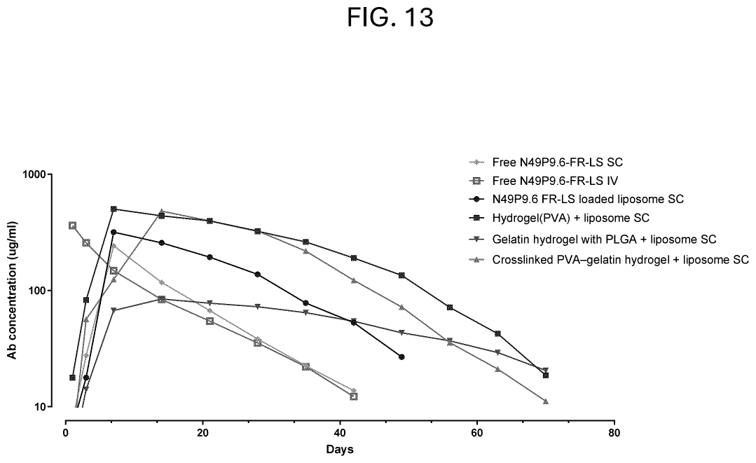

IV and SC injection of the mAb N49P9.6-FR-LS show typical peak and decline of mabs in the hFcRN model, as previously reported. Encapsulating the mAbs in liposomes improves the PK (bioavailability, and slight improvement in half-life), as previously reported. When N49P9.6-FR-LS loaded liposomes are administered subcutaneously, there is a characteristic 1 week lag until peak titers are achieved, followed by a moderate and continuous decline (FIG. 13). IV injection achieves an immediate peak, with antibody levels gradually taper off over the course of 20 to 30 days. In both SC and IV injection the antibody decay follows 1^{s} order kinetics, with the half-life of IV being 9 days, and SC slightly less at 7 days (Table 9), likely because of degradation in situ and/or transit through lymph system. Liposomal loading improves and equalizes the half-lives of the same antibodies (˜10 days), due to protection of the liposomal mAbs through the lymph transit, as previously reported (Karimi et al., Acta Biomaterialia, (2025), 195:522-535).

Further embedding the liposomal antibody formulations in hydrogels significantly improved the half-lives. The non-crosslinked PVA-gelatin hydrogel formulation combined with liposomes demonstrates moderately improved antibody retention. As expected from the in vitro studies, the crosslinked network provides diffusion control and enhances matrix stability through genipin crosslinking, which reduces porosity and enables sustained release. The non-crosslinked PVA-gelatin liposomal mAbs injected SC had a peak at 7 days, followed by decline. The non-crosslinked PVA hydrogel+liposomal formulation demonstrated the highest Cmax (658 g/mL) and AUC (10,758.5 μg·mL⁻¹·day) among all groups (Table 9). The half-life in the first month after the peak was 27.9 days with a mean residence time of 39.5 days (Table 9). Clearance was reduced to 0.03 g/(g/mL)/day, indicating prolonged systemic retention (Table 9). Overall, this formulation provided the greatest systemic exposure (AUC) for the duration tested.

The crosslinked PVA-gelatin hydrogel liposomal mAb formulation showed a delayed peak at 14 days, with a Cmax of 380 g/mL and a total AUC of 8466.5 μg·mL⁻¹·day (Table 9). The half-life in the first month after the peak was 14.2 days, with a mean residence time (MRT) of 27.2 days, indicating improved retention compared to the liposome-only group, but lower Cmax, AUC, half-life and MRT compared to the non-crosslinked PVA liposome group (Table 9). The clearance rate was reduced to 0.09 g/(g/mL)/day, consistent with the stabilizing effect of genipin crosslinking (Table 9).

The gelatin hydrogel with PLGA microspheres liposomal mAb formulation showed the lowest Cmax (85 g/mL) and AUC (2681 μg·mL⁻¹·day) due to a lower injected dose (˜400 g/mouse) compared to 1 mg in other groups. When normalized to 1000 ug injection, the Cmax was calculated to be 213 μg/mL and AUC 6702 μg·mL⁻¹·day (Table 9). This formulation also demonstrated a delayed peak (Day 14), but had the longest half-life (45 days) and mean residence time (58.0 days) of all the formulations (Table 9), indicating highly prolonged antibody retention. The clearance rate was 0.07 g/(g/mL)/day (Table 9), reflecting sustained systemic exposure. This dual-barrier system demonstrated excellent long-acting potential, even at a reduced dose.

This results in a steady decline in antibody concentration, with prolonged exposure over time. However, the in vivo data reveal that the non-crosslinked PVA hydrogel formulation actually provides a more sustained release than the crosslinked version, suggesting differences in hydrogel behavior between in vitro and in vivo environments. The most prolonged and stable PK profile is seen with the gelatin hydrogel embedded with PLGA microspheres and liposomes. This dual-barrier system offers both diffusion resistance from the hydrogel and gradual degradation-mediated release from the PLGA microspheres. Antibody levels remain consistently higher and decline at the slowest rate over the entire 80-day study period. Overall, the data clearly demonstrate that increasing the structural complexity of the delivery system-through crosslinking and microsphere encapsulation-leads to enhanced control over release kinetics and superior sustained antibody exposure following SC administration.

TABLE 9
Pharmacokinetic (PK) parameters of mAb-loaded liposomal hydrogel formulations administered SC

| | | | | ΔAUC | ΔAUC | | Δt_{1/2} | Δt_{1/2} | | CL |
|---|---|---|---|---|---|---|---|---|---|---|
| | | Cmax | AUC₀₋ₜ | (vs. | (vs. | t_{1/2} | (vs. | (vs. | MRT | (μg/(μg/ |
| Formulation | Injection | (μg/ml) | (μg · d/mL) | IV)% | SC)% | (d) | IV)% | SC)% | (d) | ml)/d) |
| Free N49P9.6-FR-LS | IV | 510.25 | 3850 | — | — | 9.0 | | — | 11.5 | 0.25 |
| Free N49P9.6-FR-LS | SC | 242.25 | 3260 | −15% | — | 7.1 | −22 | — | 16.1 | 0.29 |
| Liposome-loaded | SC | 292.9 | 5641.3 | +46% | +73% | 12.0 | +33.1 | +70.3 | 20.5 | 0.159 |
| N49P9.6 FR-LS | | | | | | | | | | |
| PVA Hydrogel + | SC | 658 | 10758.5 | +179% | +230% | 27.9 | +210.2 | +293.2 | 39.5 | 0.03 |
| Liposome | | | | | | | | | | |
| Crosslinked PVA-Gelatin | SC | 380 | 8466.5 | +119% | +160% | 14.2 | +55% | +100% | 27.2 | 0.09 |
| Hydrogel + Liposome | | | | | | | | | | |
| PLGA Microspheres | SC | 212.5 | 6702.5* | +74.1 | +105.6 | 44.7 | +396.7 | +529 | 58.0 | 0.07 |
| in Gelatin + Liposome | | | | | | | | | | |

Peak plasma concentration (Cmax), area under the curve (AUC₀₋ₜ), half-life (t_{1/2}), mean residence time (MRT), and clearance (CL).
*Data normalized by a factor of 2.5 as injection dosage was 400 ug instead of 1000 ug due to lower loading.

### Methods and Materials

### Materials

All lipids including phosphatidylcholine (PC), cholesterol, and DSPE-PEG2000 were obtained from Avanti Polar Lipids (Alabaster, AL). PLGA (50:50, MW 30-60 kDa) was purchased from Evonik. Gelatin (Type A), polyvinyl alcohol (PVA), and methylene chloride were acquired from Sigma-Aldrich. Monoclonal antibody N49P9.6-FR-LS was produced and purified in-house. All solvents were of analytical grade and sterile PBS was used throughout.

### Development of Injectable Hydrogel-Based Nanodrug Delivery Systems (NDDS)

To design a sustained-release injectable delivery system for subcutaneous (SC) administration of liposome-encapsulated antibodies, we formulated three distinct liposomal nanocomposite hydrogels (LNHs), PVA Hydrogel, Crosslinked PVA Hydrogel and PLGA Microspheres Embedded in Gelatin Hydrogel.

### Preparation of Antibody-Loaded Liposomes

PEGylated and non-PEGylated liposomes were prepared by thin-film hydration and extrusion, as described in our previous work. Lipids (PC:Cholesterol:DSPE-PEG2000; 65:30:5 for PEGylated) were dissolved in chloroform, dried into a thin film, and hydrated with PBS containing N49P9.6-FR-LS. The suspension was extruded through 400, 200, and 100 nm membranes to form uniform unilamellar liposomes. Size, PDI, and zeta potential were measured by DLS, and encapsulation efficiency was determined by ELISA after ultracentrifugation (Karimi et al., Acta Biomaterialia, (2025), 195:522-535).

Non-Crosslinked PVA Hydrogel Loaded with Liposomes

Liposomes were incorporated into a 5% (w/w) polyvinyl alcohol (PVA; MW 133 kDa) solution by loading them into 1 mL of the PVA solution using a sterile syringe. Prior to use, the PVA solution was sterilized through a 0.22 m membrane filter. The resulting suspension was briefly sonicated for 10 seconds to ensure homogeneity. To facilitate hydrogel formation, a freeze-thaw cycle was performed by placing the suspension at −20° C. for 2 hours, followed by incubation at room temperature for 1 hour. All procedures were conducted under aseptic conditions in a laminar flow hood.

### Genipin-Crosslinked PVA-Gelatin Hydrogel Containing Liposomes

To enhance mechanical stability and prolong release, the liposome-loaded hydrogel was prepared by crosslinking a PVA-gelatin blend using genipin. Liposomes were suspended in 1 mL of a sterile 5% (w/w) polyvinyl alcohol (PVA; MW 133 kDa) solution blended with 5% (w/v) gelatin. Genipin was added to the mixture at a final concentration of 0.2% (w/v) to enable crosslinking via the primary amine groups in gelatin. The PVA-gelatin solution was pre-sterilized using a 0.22 m membrane filter. The resulting liposome-containing hydrogel precursor was transferred into a sterile syringe and sonicated briefly (10 seconds) to ensure uniform dispersion. Crosslinking was initiated by incubation at 37° C. for 12 hours to allow the genipin reaction to proceed. All steps were performed under aseptic conditions in a laminar flow hood.

Gelatin Hydrogel Embedded with PLGA Microspheres Encapsulating Liposomes

Liposomes were encapsulated into poly(lactic-co-glycolic acid) (PLGA) microspheres via a modified double emulsion-solvent evaporation method. The resulting microspheres were subsequently suspended in a gelatin hydrogel matrix. PLGA (75:25 MW >100 kDa) was dissolved in methylene chloride at 20% w/v to form the organic phase. Separately, liposomes containing the broadly neutralizing antibody (bNAb) were prepared in PBS and used as the internal aqueous phase. The liposome suspension was emulsified into the PLGA solution using a high-speed homogenizer at 50° C. to form the primary water-in-oil (W/O) emulsion. This emulsion was then cooled to 15° C. and added dropwise into a 2% w/v PVA aqueous solution. The mixture was sonicated for 50 seconds to generate a stable water-in-oil-in-water (W/O/W) emulsion. The resulting emulsion was stirred in a 0.5% PVA bath at room temperature to allow methylene chloride evaporation. Microspheres were collected by centrifugation at 10,000×g for 10 minutes, washed three times with deionized water, and stored at 4° C. The harvested microspheres were suspended in a warm 10% w/v gelatin solution in PBS, maintained at 37° C. The suspension was gently stirred to ensure homogeneous distribution of microspheres throughout the gelatin matrix. The final formulation contained approximately 10 wt % liposomes and 2-4 wt % PLGA microspheres. The hydrogel was loaded into sterile syringes and kept at 4° C. until use.

### In Vitro Release Study of Liposomal Loaded Hydrogel

To evaluate the release kinetics and leakage stability of monoclonal antibody (mAb)-loaded liposomal and PLGA microsphere formulations embedded in hydrogel matrices, in vitro release studies were conducted under physiologically relevant conditions. Liposomal formulations containing 1.7 mg of mAb were incorporated into the hydrogel, while 500 g of mAb-loaded PLGA microspheres were similarly embedded within the hydrogel for comparison. Each formulation was loaded into a dialysis cassette (300 kDa MWCO, Thermo Fisher Scientific) and immersed in 9 mL of release medium consisting of 50% sterile human plasma in 5% dextrose-PBS buffer (pH 7). The cassettes were incubated at 37° C. with gentle agitation (100 rpm) under sterile conditions. At predefined time points (Days 1, 3, 7, 10, 14, and 21), 100 L of the external medium was collected and replaced with an equal volume of fresh medium to maintain sink conditions. This sampling procedure was repeated in triplicate. Collected samples were centrifuged at 4000 g for 20 minutes using 300-kDa MWCO centrifugal filter units (Ultra-4, Millipore) to isolate the released mAb.

The concentration of released mAb was measured using an indirect ELISA specific for intact human IgG. All procedures were carried out under sterile conditions, and results were reported as cumulative release percentages over time.

### In Vitro Cytotoxicity Assay

To evaluate the cytotoxicity of the mAb-loaded liposomes, an MTT assay was conducted. HMEC-1 cells were seeded into 96-well plates at a density of 5×10³ cells per well and allowed to adhere overnight at 37° C. The next day, cells were treated with varying concentrations of the liposomal formulations and incubated for 24, 48, and 72 hours. After each incubation period, the medium was removed, and cells were gently washed with PBS. Subsequently, MTT reagent was added and incubated for 4 hours at 37° C. in the dark. The solution was then replaced with 200 L of DMSO to dissolve the formazan crystals. Absorbance was measured at 570 nm using a microplate reader. Each experimental condition was tested in triplicate. The IC₅₀ values were calculated using CalcuSyn v2 software (BIOSOFT, UK). Cell viability was determined using the following formula:

% Viability=[(Mean OD_sample −Mean OD_blank)/(Mean OD_control −Mean OD_blank)]×100

To further assess cytotoxic effects, apoptosis was measured via caspase-3 activation using flow cytometry. After treatment, cells were harvested and stained with Red-DEVD-FMK, a cell-permeable caspase-3 inhibitor conjugated with sulfo-rhodamine (CaspGLOW Red Active Caspase-3 Staining Kit, BioVision). Cells heat-treated at 55° C. for 7 minutes served as a positive control. Staining was performed at 37° C. for 45 minutes, followed by three PBS washes. Samples were analyzed using a BD FACSCanto II cytometer and FlowJo v8.5 software (BD Biosciences).

### Pharmacokinetics & Pharmacodynamics Study

All animal procedures were conducted in accordance with protocols approved by the Institutional Animal Care and Use Committee (IACUC) at the University of Maryland, Baltimore. Pharmacokinetic (PK) studies were performed in groups of 4-5 humanized FcRn-knockout transgenic (FcRn−/− hFcRn Tg) SCID mice (6 weeks old, 19-26 g), with equal distribution of male and female animals per group. Mice received 30 mg/kg of monoclonal antibody (mAb), either in free form or encapsulated in liposomes, administered via intravenous (IV) injection through the retroorbital vein or subcutaneous (SC) injection at the flank. mAb concentrations were quantified prior to dosing using BCA assay to ensure consistent dosing across groups. Blood samples were collected at baseline (pre-dose) and on Days 1, 3, 7, and weekly thereafter. Samples were collected in EDTA-coated microcapillary tubes and centrifuged at 2000 g for 10 minutes at 4° C. Plasma was separated and stored at −80° C. until analysis. mAb concentrations in plasma were determined by indirect ELISA. Plates were coated with unlabeled anti-human kappa and lambda antibodies (1 g/mL in TBS) and incubated overnight at 4° C. Following blocking with Blotto/NP-40, diluted plasma samples and standards were added, followed by HRP-conjugated detection antibody and substrate development. Absorbance was measured at 620 nm using a SpectraMax Paradigm reader (Molecular Devices). All data were fitted to a 4-parameter logistic model. Pharmacokinetic parameters including Cmax, AUC, t½, mean residence time (MRT), and clearance (CL) were calculated using non-compartmental analysis via PKSolver.

### Conclusion

The development of effective long-acting subcutaneous (SC) delivery systems for monoclonal antibodies (mAbs) is a significant step toward improving therapeutic outcomes, reducing injection frequency, and enhancing patient adherence. In this study, we evaluated three hydrogel-based delivery platforms non-crosslinked PVA hydrogel, genipin-crosslinked PVA-gelatin hydrogel, and gelatin hydrogel containing PLGA microsphereseach designed to sustain the release of liposome-encapsulated broadly neutralizing antibodies (bnAbs) following SC administration. Polyvinyl alcohol (PVA) was selected as a base hydrogel material due to its biocompatibility, water solubility, and shear-thinning behavior, which makes it suitable for injectable formulations. PVA can form physical hydrogels via freeze-thaw cycling, enabling entrapment of liposomes without chemical modification. However, physical gels formed by PVA alone lack long-term mechanical strength and are susceptible to rapid degradation under physiological conditions, limiting their effectiveness for sustained release. To overcome these limitations, we developed a chemically crosslinked hydrogel by blending PVA with gelatin and introducing genipin as a biocompatible crosslinker. Gelatin contributes bioactivity and tissue adhesion properties, while genipin crosslinking creates covalent bonds between amine groups in gelatin and hydroxyl groups in PVA, leading to a denser and more stable network. This modification enhances matrix rigidity, reduces porosity, and imparts better control over liposome release. The resulting crosslinked PVA-gelatin hydrogel is expected to degrade more slowly in vivo, maintaining depot integrity and enabling prolonged antibody release. In the third platform, we incorporated liposome-loaded PLGA microspheres into a gelatin hydrogel. PLGA (poly(lactic-co-glycolic acid)) was used with a 75:25 lactic-to-glycolic acid ratio and high molecular weight (>100 kDa) to provide extended degradation over several months. This particular ratio favors hydrophobicity and slower hydrolysis, enabling a release profile that can extend up to 4-6 months depending on formulation parameters. By embedding these microspheres in a gelatin matrix, we created a dual-barrier system: the gelatin hydrogel provides immediate structural support and localizes the microspheres at the injection site, while the PLGA core offers long-term, degradation-controlled release. This multilayered design was intended to produce the most prolonged and consistent pharmacokinetic profile among the tested platforms.

The in vitro release profiles clearly demonstrated a structure-dependent control of mAb release kinetics. Free mAbs and liposomes without a hydrogel matrix exhibited the fastest release, underscoring the need for a structural barrier to slow down diffusion. Incorporating liposomes into a non-crosslinked PVA hydrogel modestly improved retention in vitro, attributable to the formation of a loose polymeric network. However, the absence of crosslinking limited the mechanical integrity and barrier function of the matrix.

In contrast, the genipin-crosslinked PVA-gelatin hydrogel showed significantly more sustained in vitro release, releasing only −21% of the mAb over 21 days. This behavior is consistent with the formation of a tighter, chemically crosslinked network that restricts pore size and enhances hydrogel stability. Genipin crosslinking introduces covalent bonds between amine groups in gelatin and hydroxyl groups in PVA, thereby increasing resistance to enzymatic degradation and providing a more effective diffusion barrier.

The most controlled in vitro release was observed in the PLGA microsphere-embedded gelatin hydrogel, which released only 13% of the mAb over the same period. This dual-barrier system benefits from both the diffusion resistance of the gelatin matrix and the slow, degradation-mediated release of the PLGA microspheres. The combination of these mechanisms creates a sophisticated depot capable of prolonging drug exposure and minimizing burst release.

Interestingly, the in vivo pharmacokinetic profiles deviated in part from the in vitro findings. The IV and SC administration of free antibodies and their liposomal formulations yielded peaks and decays as expected by the 1st order kinetics, depending on their injection site. The half-lives ranged from 7 to 14 days, with the release pattern reflecting the absence of a structural matrix to control the diffusion and retention of the liposomes at the injection site. Without a hydrogel or microsphere to act as a barrier, the liposomes are more readily absorbed into the systemic circulation, leading to quicker clearance and a shorter duration of sustained antibody levels. While not as abrupt as a burst release, the lack of a controlled-release mechanism limits the formulation's ability to maintain prolonged therapeutic concentrations.

Unexpectedly, the non-crosslinked PVA hydrogel group exhibited more prolonged antibody retention in vivo compared to the crosslinked PVA-gelatin hydrogel, suggesting a complex interplay between matrix degradation, enzyme sensitivity, tissue interaction, and in situ gelation behavior. While crosslinking improves structural integrity in vitro, it may also reduce hydrogel biodegradability or alter swelling characteristics in vivo, potentially limiting release.

The most favorable pharmacokinetic profile was observed in the PLGA microsphere-in-gelatin formulation. Antibody levels remained relatively constant for over 80 days with an approximately 45 day half-life, demonstrating superior retention and sustained systemic exposure. This suggests that dosing intervals in this mouse could be as infrequent as every 6 months for an mAb that typically has a half-life of 7 days when injected SC. Such a gain in this system has large implications for human mAb delivery, as IgG antibody half-lives are much longer in humans than FcRn mice. Of note, the antibody decay curves for the liposome and hydrogel formulations is not a simple 1^{s} order kinetics. Its initial behavior appears as non-linear elimination that may be the result of several independent 1^{st} order reactions (half-life of free mAb in circulation, half-life of liposome subcutaneously, half-life of liposome in systemic circulation, half-life of hydrogel). This is an area that is under investigation that will likely be the subject of a future study.

The above findings confirms that hierarchical structural complexity not only in matrix composition but also through the incorporation of biodegradable microspheres plays a critical role in fine-tuning the release profile and enhancing in vivo performance. However, despite its extended release capability, a major limitation of this system is its low antibody loading efficiency, which was consistently measured at only 10-15%. This contrasts with the 55% or higher loading efficiencies typically achieved in direct hydrogel-based systems that do not incorporate microspheres. Such low encapsulation efficiency not only reduces the amount of active antibody available for therapeutic effect, but also significantly increases the material and production cost per effective dose. This inefficiency stems from multiple factors, including potential loss of liposomes during the double emulsion process, incomplete entrapment within the PLGA matrix, and diffusion into the aqueous phase during solvent evaporation. While microsphere-based systems are ideal for long-term delivery, these losses pose a major translational barrier particularly for expensive biologics such as monoclonal antibodies; however, in this study we did not optimized the loading conditions so its upper limit remains unknown. By contrast, hydrogel only formulations offer higher encapsulation efficiencies, simpler preparation, and better scalability, albeit with shorter release durations. Therefore, the choice of delivery platform must balance between release longevity and loading efficiency, considering both therapeutic goals and cost-effectiveness for clinical translation. Optimization strategies such as improving emulsion stability, adjusting PLGA properties, or considering alternative preparation methods beyond the W/O/W technique may be necessary to enhance the efficiency and economic viability of microsphere-based systems.

In summary, we describe a method that can significantly enhance mAb half-lives with a combination of liposomal formulations coupled with slow-release via hydrogels.

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

1. A liposomal pharmaceutical formulation for subcutaneous or intravenous administration of antibodies, comprising
a. one or more phospholipids;
b. one or more antibodies; and
c. a pharmaceutically acceptable vehicle,
wherein the one or more phospholipids form liposomes that encapsulate the one or more antibodies, wherein the liposomes have a polydispersity index (PDI) below 0.2 and at least 90% of the liposomes have a size less than 125 nm.

2. The liposomal pharmaceutical formulation of claim 1, wherein the liposomes further comprise an effective amount of cholesterol or a derivative thereof.

3. The liposomal pharmaceutical formulation of claim 1, wherein 90% of the liposomes have a size ranging from 75 nm to 100 nm.

4. The liposomal pharmaceutical formulation of claim 1, wherein 80% of the liposomes have a size ranging from 92 nm+/−3.6 nm.

5. The liposomal pharmaceutical formulation of claim 1, wherein 80% of the liposomes have a size ranging from 85 nm+/−5.8 nm.

6. The liposomal pharmaceutical formulation of claim 1, wherein the liposomes have a negative zeta potential, wherein the zeta potential is from −35 mV to −50 mV.

7. (canceled)

8. (canceled)

9. The liposomal pharmaceutical formulation of claim 1, wherein the zeta potential is from −10 mV to −20 mV.

10. The liposomal pharmaceutical formulation of claim 1, wherein the zeta potential is from −14.5 mV+/−2.7 mV.

11.-16. (canceled)

17. The liposomal pharmaceutical formulation of claim 1, wherein the liposomes have an encapsulation efficiency of at least 55%.

18. The liposomal pharmaceutical formulation of claim 1, wherein the liposomes comprise HSPC, DSPG, cholesterol and DSPE-mPEG2000, wherein the molar ratio of HSPC:cholesterol:DSPE-mPEG2000:DSPG is from about 40-70:25-45:2-15:1-20.

19. (canceled)

20. (canceled)

21. The liposomal pharmaceutical formulation of claim 1, wherein the liposomes comprise HSPC, cholesterol and DSPG, wherein the molar ratio of HSPC:cholesterol:DSPG is about 40-80:10-30:10-30.

22.-24. (canceled)

25. The liposomal pharmaceutical formulation of claim 1, wherein the liposomes are embedded or encapsulated in an injectable matrix.

26. The liposomal pharmaceutical formulation of claim 25, wherein the liposomes are embedded in microspheres.

27. (canceled)

28. (canceled)

29. The liposomal pharmaceutical formulation of claim 26, wherein the microspheres are embedded in a hydrogel.

30. (canceled)

31. The liposomal pharmaceutical formulation of claim 1, wherein the liposomes are embedded or encapsulated in an injectable silica matrix.

32. The liposomal pharmaceutical formulation of claim 1, wherein the liposomes are embedded in an injectable hydrogel matrix, wherein the injectable hydrogel matrix comprises a blend of poly(vinyl alcohol) (PVA) and gelatin, wherein the injectable matrix is chemically or physically crosslinked, wherein the injectable matrix is crosslinked using genipin.

33.-43. (canceled)

44. A method of treating or preventing a disease or condition in a subject, comprising administering subcutaneously or intravenously to the subject an effective amount of the liposomal pharmaceutical formulation of claim 1.

45. (canceled)

46. The method of claim 44, wherein release of the antibody into the subject's plasma is delayed and then sustained over a period of time.

47. The method of claim 44, wherein the antibody achieves a peak plasma concentration at least 7 days following administration in the subject.

48. (canceled)

49. (canceled)

50. The method of claim 44, wherein the antibody has a half-life in the subject that is at least 30% greater than the half-life of the free antibody in a control subject administered an intravenous injection of the free antibody.

51.-53. (canceled)


SEQUENCE LISTING (546 sequences)

SEQ ID NO: 1 - protein, 458 aa, Homo sapiens
AGLMQSGAVMKNSGASVRVSCQADGYDFIDYVIHWFRQRRGEGLEWLGWMNPSGGGTNYPRPFQGKVTMTRDTSTETAYLDVRGLTYDDTAVYYCVRDRANGSGRRRFESVNWFLDLWGRGTQITVVSPSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 205 aa, Homo sapiens
QSALTQPRSVSASPGQSVTISCTGTHNYVSWCQQKPGQAPKLLIYDFNKRPSGVSDRFSGSTSGNTASLTISGLQADDEGHYFCWAFENIGGGTKLTVLRQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 3 - protein, 457 aa, Homo sapiens
ADLVQSGAVVKKPGDSVRISCEAQGYRFPDYIIHWIRRAPGQGPEWMGWMNPMGGQVNIPWKFQGRVSMTRDTSIETAFLDLRGLKSDDTAVYYCVRDRSNGSGKRFESSNWFLDLWGRGTAVTIQSSSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 203 aa, Homo sapiens
ALTQPRSVSASPGQSVTISCTGTHNLVSWCQHQPGRAPKLLIYDFNKRPSGVPDRFSGSGSGGTASLTITGLQDDDDAEYFCWAYEAFGGGTKLTVLRQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 5 - protein, 444 aa, Homo sapiens
ADLVQSGAVVKKPGDSVRISCEAQGYKFPDYIIHWIRRAPGQGLEWMGWINPMGGQVNIPWQFQGRVSMTRDTSIETAFLDLRGLKSDDTALYYCVRDRSNGSGRRFESSNWFLDLWGRGTAVTVHSPSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 205 aa, Homo sapiens
QSALTQPRSVSASPGQSVTISCTGTHNLVSWCQHHPGRAPKLLIYDFNKRPSGVPDRFSGSGSGGTASLTISGLQDDDDAEYFCWAYEAFGGGTKLTVLRQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 7 - protein, 458 aa, Homo sapiens
SAELVQSGAVVKKPGTSVKVSCQAYGYTFTDYLIHWLRQAPGQGLEWMGWMNPVYGQVNYAQNFQGRVSMTRDIYRETAFLEVRDLKTDDTGTYYCVRDTGDGSRRHFDSINWFLDLWGRGTWIRVAPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 205 aa, Homo sapiens
QCVLTQPRSVSGSPGQSVTISCTGTHNYVSWCQHHPGNAPKLLLYDFDKRPSGISDRFSGSRSGNTASLTISGLQPEDEADYFCWAFEAFGGGTKVLVLRQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 9 - protein, 457 aa, Homo sapiens
ADLVQSGAVMKKPGDSVRISCEARGYTFTDYVIHWIRRAPGQGLEWMGWIDPPYGQVNIPWNFQGRVSMTRDTSIETAFLDLRGLKSDDTGLYYCVRDRSNGWGKRFESSNWFLDLWGRGTVVTVHSPSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 205 aa, Homo sapiens; identical to SEQ ID NO: 6

SEQ ID NO: 11 - protein, 457 aa, Homo sapiens
ADLVQSGAVVKKPGDSVRISCEAQGYTFTDYVIHWIRRAPGQGLEWMGWINPGYGQVNIPWNFQGRVSMTRDTSIETAFLDLRGLKSDDTGLYYCVRDRSNGWGKRFESSNWFLDLWGRGTVVTVHSPSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 205 aa, Homo sapiens
QSALTQPRSMSASPGQSVTISCTGTHNLVSWCQHHPGRPPKLLIYDFNKRPSGVPDRFSGSGSGGTASLTISGLQDDDDAEYICWAYEAFGGGTKLTVLRQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 13 - protein, 457 aa, Homo sapiens
ADLVQSGAVVKNAGASVRVSCEAYGYTFVDYFIHWVRQAPGQGFEWMGYMDPLNGRPNIARKFQGRLSLSRDRSSETSFLDLSGLRSDDSAVYYCVRDKSNGSGRRFDSSNWFLDLWGRGTRVSIFSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 205 aa, Homo sapiens
QSALTQPRSVSATPGQSVTISCTGTHNYVSWCQQHPGRAPKLLIYDFNKRPSGVPDRFSGSGSGGTASLTITGLQDDDEADYFCWAYDAFGGGTKLTVLRQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 15 - protein, 457 aa, Homo sapiens
ADLVQSGAVVKKPGDSVRVSCEAYGYTFSDYIIHWIRRAPGRGLEWMGWMNPMGGQVNIPWNFQGRVSMTRDTSIETAFLDLRGLRSDDTAVYYCVRDRSNGSGKRFESSNWFLDLWGRGTAVTISSPSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 205 aa, Homo sapiens
QSALTQPRSVSAAPGQSVTISCTGTHNLVSWCQHHPGRAPKLLIYDFNKRPSGVPDRFSGSGSGGTASLTITGLQDDDEAEYFCWAYEVFGGGTKLTVLRQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 17 - protein, 457 aa, Homo sapiens
ADLVQSGAVVKTPGASVRVSCEAYGYTFIDYIIHWVRQAPGQGFEWLGYIDPMNGRPNIARKFQGRLSLSRDTSIETSFLDLSGLRSDDSAVYYCVRDKSNGSGKRFDSSNWFLDLWGRGTRVSISSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 205 aa, Homo sapiens
QSALTQPRSVSAAPGQSVTISCTGTHNYVSWCQQHPGRAPKLLIYDFNKRPSGVPDRFSGSGSGGTASLTITRLQDDDDADYFCWAYDAFGGGTKLTVLRQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 19 - protein, 457 aa, Homo sapiens
ADLVQSGAVVKKPGASVRVSCEAYGYTFTDYIIHWIRQAPGQGLEWMGWMNPMGGRTNIPWKFQGRVSMTRDTSIETAFLDLSGLTSDDTAVYYCVRDKSNGSGKRFDSSNWFLDLWGRGTPVTISSPSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 205 aa, Homo sapiens
QSALTQPRSVSAAPGQSVTISCTGTHNLVSWCQQHPGRAPKLLIYDFNKRPSGVPDRFSGSGSGGTASLSITGLQDDDEAEYFCWAYEAFGGGTKLTVLRQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 21 - protein, 457 aa, Homo sapiens
ADLVQSGAVVKKPGASVRVSCEAYGYTFVDYLIHWVRQAPGQGFEWMGYMDPMNGRPNIARKFQGRLSLSRDTSIETSFLDLSGLRSDDSAVYYCVRDKSGGSGKLFDSSNWFLDLWGRGTRVSISSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 205 aa, Homo sapiens
QSALTQPRSVSAAPGQSVTISCTGTHNYVSWCQQHPGRAPKLLIYDFNKRPSGVPDRFSGSGSGGTASLTITGLQDDDDADYFCWAYDAFGGGTKLTVLRQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 23 - protein, 457 aa, Homo sapiens
ADLVQSGAVVKKPGDSVRISCEAQGYTFTDYVIHWIRRAPGQGLEWMGWINPGYGQVNIPWNFQGRVSMTRDTSIETAFLELRGLKSDDTGLYYCVRDRSNGWGKRFESSNWFLDLWGRGTVITVHSPSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 205 aa, Homo sapiens
QSALTQPRSMSASPGQSVTISCTGTHNLVSWCQHHPGRPPKLLIYDFNKRPSGVPDRFSGSGSGGTASLTITGLQDDDDAEYICWAYEAFGGGTKLTILRQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 25 - protein, 457 aa, Homo sapiens
ADLVQSGAVVKKPGDSVRISCEAQGYTFTDYVIHWIRRAPGQGLEWMGWMDPSYGQVNIPRNFQGRVSMTRDTFRETAYLELRGLQSDDKGLYYCVRDRSHGSGRQFESSNWFLDLWGRGTVVNVQSPSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 205 aa, Homo sapiens
QSALTQPRSVSASPGQSVTISCTGTHNLVSWCQHHPGRPPKLLIYDFNKRASGVPDRFSGSGSGGTASLTISGLQDDDDAEYFCWAYEAFGGGTKLTVLRQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 27 - protein, 457 aa, Homo sapiens
ADLVQSGAVVKKPGDSLRISCEAQGYTFTDYVIHWIRRAPGQGLEWMGWMDPSFGQMNIPRNFQGRVSMTRDMYIETAFLDLRGLKSDDTGLYYCVRDRSHGSGRLFESSNWFLDLWGRGTVVTVQSPSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 205 aa, Homo sapiens; identical to SEQ ID NO: 26

SEQ ID NO: 29 - protein, 457 aa, Homo sapiens
ADLVQSGAVVKKPGDSVRISCEAQGYRFTDYVIHWIRRAPGQGLEWMGLMDPSFGRMNIPRKFQGRVSMTRDTSMETAFLDFRGLNFDDTGLYYCVRDRSHGSGRLFESSNWFLDLWGRGTVVTVQSPSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 205 aa, Homo sapiens; identical to SEQ ID NO: 26

SEQ ID NO: 31 - protein, 457 aa, Homo sapiens
ADLVQSGAVVKKPGDSVRISCEAQGYTFIDYVIHWIRRAPGQGLEWMGLMDPTYGRMNIPRKFQGRVSMTRDTSIETAFLDLRGLKSDDTGLYYCVRDRSHGSGRLFESSNWFLDLWGRGTVVTVQSPSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 205 aa, Homo sapiens; identical to SEQ ID NO: 26

SEQ ID NO: 33 - protein, 457 aa, Homo sapiens
ADLVQSGAVVKKPGDSVRISCEAQGYRFLDYIIHWIRRAPGQGLEWMGWMNPMGGQVNIPWNFQGRVSMTRDTSIETAFLDLRGLKSDDTAVYYCVRDRSNGSGKRFESSNWFLDLWGRGTAVTIHSPSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 205 aa, Homo sapiens
QSALTQPRSVSASPGQSVTISCTGTHNLVSWCQHHPGRAPKLLIYDFNKRPSGVPDRFSGSGSGGTASLTITGLQDDDDAEYFCWAYEAFGGGTKLTVLRQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 35 - protein, 450 aa, Homo sapiens
HVQLVQSGGGVKKIGAAVRISCEVTGYKFMDQLINWVRQAPGQGLEWMGWMNPTYGQVNYSWRFEGRVTMTRDMDTETAFMELRGLRVDDTAVYYCARGPSGENYPFHYWGQGVRVVVSSPSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 206 aa, Homo sapiens
ASALTQPASMSASPGQSVTISCSGTRHIISAWFQQYPGKPPKLIIFDDDKRPSGVPSRFSASRPGDTASLTISNVQPEDEATYICNTYEFFGGGTRLTVLSQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 37 - protein, 450 aa, Homo sapiens
HIQLLQSGPQVKKSGDTVRISCETSGYNFVDSRIHWVRQTPEKRLRWMGWINPLQGGVNYAPEFQGRIRMTRDTFIDTVYVDLSGLTPADTAYYYCARGIDGKSYPFHFWGHGTRVTVFSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 203 aa, Homo sapiens
RFALTQPASVSGSPGQTITITCAGGSVSWFHFPPGKTPRLIIYESSKRPSGVSPRFSGSQSGSTASLIISGLQSDDEGTYFCSILEFFGRGTLVTVLSQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 39 - protein, 453 aa, Homo sapiens
QVRLVQSGAGARKTGASMKLSCSTSGYTFTTHHGHFINWVRQARGQGLEWMGWMNPMTGQMNIEGKFQGRVTLTRDIYSDTAYMEMTRLTTGDTGTYYCARGDFGQNYPFHYWGQGSLVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 206 aa, Homo sapiens
LSALTQPASVSGSPGQSVTISCSGTNRYLVSWYQQHPDKAPKLIIYDDNKRPSGISDRFSASRPDDTASLTISGLQTGDEATYWCASYERFGGGTRLTVLSQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 41 - protein, 450 aa, Homo sapiens
HVQLVQSGGGVKKIGAAVRISCEVSGYNFMDQFINWVRQAPGQGLEWMGWMNPIYGQVNYSWRFQGRVTMTRDMYTDTAFMELRGLRVDDTAVYYCARGPSGENYPFHYWGQGVRVVVSSPSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 203 aa, Homo sapiens
LTQPASMSASPGQSVTISCSGTRHIISAWFQQYPGKPPKLIIFDDDKRPSGVPSRFSASRPGDTASLTISNVQPEDEATYICNTYEFFGGGTKLTVLSQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 43 - protein, 450 aa, Homo sapiens
HIQLLQSGPQVKKSGDTVRISCETSGYNFVDSRIHWVRQTPEKRLRWMGWINPLHGGVNYAPEFQGRIRMTRDTFIDTVYVDLSGLTPADTAYYYCARGIDGKSYPFHFWGHGTRVTVFSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 203 aa, Homo sapiens; identical to SEQ ID NO: 38

SEQ ID NO: 45 - protein, 455 aa, Homo sapiens
RVTLQQSGAIVRQPGASVTVSCETSGYTFTKYFIYWVRQAPGQGLEWLGRIHPRTGAVKYAPRFQGRLSMTRDWSLDTAYLGLTGLTLGDTALYFCARGAFEADSYGSSYPFHHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 207 aa, Homo sapiens
SWALTQPASVSASPGQSVTMSCTGFGNYNPDSWYQQYPGKAPKLIIYEDNKRPSGVSDRFSASRLGSTSSLTISNVQAADDAHYVCASFEFFGGGTKLTVLSQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 47 - protein, 455 aa, Homo sapiens
RVTLQQSGATVKQPGASVTVSCETSGYTFTKYTIHWVRQAPGQGLQWVGRIHPRTGAVKYAPIFQGKVSMSRDLSRDTAYLGLTRLTLADTALFFCARGAFEADLSGPTYPFHHWGQGTLVIVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 207 aa, Homo sapiens
SWALTQPASVSASPGQSVTMSCTGFGNYNPDSWYQQYPGKAPKLIIYEDNKRPSGVSNRFSASRLGSTSSLTISNVQAADDAHYVCASFEFFGGGTKLIVLSQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 49 - protein, 450 aa, Homo sapiens
NVQLMQSGTEVKKSGASVTISCETAGFNFIDSVIHWLRQAPGGGFQWMGWIKPLRGAVNYPQFLQGRVSMTRDLSTDTVYMVLNGLTPDDTGLYYCAKGAFRGGSPFGFWGQGTLLTVSPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 202 aa, Homo sapiens
QSALSQPVSVSGSPGESITISCTGATTWYQQLPGRPPKLIIYDVTNRPSGISSRFSGSTSGHTASLTISGLQVDDEGLYHCASREFFGGGTKLTVLSQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 51 - protein, 450 aa, Homo sapiens
QVRLVQSGPQVKKTGASVRVSCETSGYTFTSYFIHWLRLGPGEGLQWMGWINPLHGAVNYENKFRGRVTITRDTSTDTVYLDMSRLTPDDTAVYFCTRGIVADGWPYGHWGQGTQVTVSPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 203 aa, Homo sapiens
SWALTQPASVSGSPGQSVAISCAGGSVSWYQVLPGRAPKLIIYEGAKRPSGVSARFSGSQSGNTAYLTISDLQTEDEGIYFCSSLQFFGGGTKLTVLSQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 53 - protein, 451 aa, Homo sapiens
QVRLQQSGVVVRKPGASVRISCETSGFTFIDHIVHWVRRAPGRGFEWMGWIKPLRGAVDYAPQLRGRISLTRDIYSETVFIDVSRLTSGDTAIYFCCKAAAPEEAFPLQYWGQGTQLIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 205 aa, Homo sapiens
QAALTQPASVSGSPGQSVTISCLYANVDICWYQLHPGRAPKLLIVDNNKRPSGVSPRFSGSKSGTTASLTISGLQADDEAEYHCSSRTFFGGGTKLTVLSQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 55 - protein, 451 aa, Homo sapiens
QVRLQQSGVVVRKPGASVRLSCETSGFKFIDHIVNWVRRAPGRGFEWMGWIKPLGGVADYAPQHRGRISLTRDIYTETVFIDLSRLTSGDTAIYFCCKAAAPDEAFPLEYWGQGTQLIVSPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 205 aa, Homo sapiens
QAALTQPASVSGSPGQSVTISCLYANVDICWYQIQPGRLPKLLIVDNNRRPSGVSPRFSGSKSGTTASLTISGLQADDEAEYHCSSTTFFGGGTKLTVLSQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 57 - protein, 451 aa, Homo sapiens
QVRLVQSGPVMRKPGASVRISCETSGFAFLDHIVHWVRRAPGRGFEWMGWVKTIGGVVDYAPHLRGRISVTRDVFSETVFLDLSRLTSGDTAMYFCSKAAAPDEAFPLEFWGQGTQVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 205 aa, Homo sapiens
QAALTQPASVSGSPGQSVTISCLYANVDICWYQLHPGRAPKLLILDNNKRPSGVSSRFSGSKSGTTASLTISDLQADDEAEYHCSSTTFFGGGTRLTVLSQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 59 - protein, 451 aa, Homo sapiens
QVRLVQSGPVVRKPGTSVRISCETSGFAFLDHIVHWVRRAPGRGFEWMGWVKTIGGVVDYAPHLRGRISVTRDVFSEIVFMELSRLTSGDTAMYFCSKAAAPDEAFPLEFWGQGTQVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 205 aa, Homo sapiens
QAALTQPASVSGSPGQSVTISCLYANVDICWYQLHPGRAPKLLIVDNNKRPSGVSSRFSGSKSGTTASLTISDLQADDEAEYHCSSTTFFGGGTRLTVLSQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 61 - protein, 450 aa, Homo sapiens
QVQLVQSGAGVKKPGASVRVSCETSGFKFTEYFIHFLRQAPGQGLEWMGWLNPLRGAVNYPRKFQGRVTLTRDIYTTTVYMQLNGLTPDDTAVYYCARAVFNEAFPFDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 204 aa, Homo sapiens
SWAQTQPASVSGSPGQSITISCAGIVSDAWYQQYPGRPPRLILYDGDKRPSGVSPRFSASRAGKTASLTISGLQADDEAYYHCASREFFGGVTKLTVLSQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 63 - protein, 451 aa, Homo sapiens; identical to SEQ ID NO: 55

SEQ ID NO: 64 - protein, 205 aa, Homo sapiens
QAALTQPASVSGSPGQSVTISCLYANVDICWYQIQPGRLPKLLIVDNDRRPSGVSPRFSGSKSGTTASLTISGLQADDEAEYHCSSTTFFGGGTKLTVLSQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 65 - protein, 451 aa, Homo sapiens; identical to SEQ ID NO: 57

SEQ ID NO: 66 - protein, 205 aa, Homo sapiens; identical to SEQ ID NO: 58

SEQ ID NO: 67 - protein, 450 aa, Homo sapiens
HVQLRQSGTEAKKSGASVTISCETAGFNFIDSVIHWLRQAPGGGFQWMGWIKPLRGGVNYPHYLQGRISMTRDLSSDTVYMVLNRLTPADTGLYYCAKGAFGGSSPFGFWGQGTLLTVSPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - protein, 202 aa, Homo sapiens
QSALSQPVSVSGSPGESITISCTEATTWYQQLPGKPPKLIIYDVTNRPSGISSRFSGSMSGRTASLTISGLQVDDEGLYHCASREFFGGGTKLTVLSQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 69 - protein, 450 aa, Homo sapiens
HVQLMQSGTQAKKSGASVTISCETAGFKFIDSVIHWLRQAPGGGFQWMGWIKPLGGAVNYPPYLQGRISLTRDLSTDTIYMVLNGLTPADTGFYYCAKGAFGGGSPFGFWGQGTLLTVSPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - protein, 202 aa, Homo sapiens
QSALSQPVSVSGSPGDSITISCFGATTWYQQLPGRPPKLIIYDVTNRPSGISGRFSGSMSGQKASLTISGLQVDDEGLYHCASREFFGGGTKLTVLSQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 71 - protein, 450 aa, Homo sapiens
HVQLRQSGTEAKKSGASVTISCETAGFNFIDSVIHWLRQAPGGGFQWMGWIKPLRGGVNYPHYLQGRISMTRDLSSDTVYMVLNRLTPDDTGLYYCAKGAFGGSSPFGFWGQGTLLTVSPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - protein, 202 aa, Homo sapiens
QSALSQPVSVSGSPGESITISCTEATTWYQQLPGRSPKLIIYDVTNRPSGISSRFSGSMSGRTASLTISGLQVDDEGLYHCASREFFGGGTKLTVLSQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 73 - protein, 455 aa, Homo sapiens
RVTLQQSGATVRQPGASVTVSCETSGFTFIKYTIHWVRQAPGQGLQWVGRIHPRTGAVKFAPIFQGKFSMSRDLSRDTAYLGLTRLTLADTALFFCARGAFEADLYGPTYPFHHWGQGTQVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - protein, 207 aa, Homo sapiens
SWALTQPASVSASPGQSVTMSCTGFGSYNPDSWYQQYPGKAPKLIIYDDNKRPSGVSDRFSASRLGSTSSLTISNVQAADDAHYVCASFEFFGGGTKLTVLSQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 75 - protein, 450 aa, Homo sapiens
HIQLLQSGPQVKKSGDTVRISCETSGYNFVDSLIHWVRQTPEKRLRWMGWINPLQGGVNYAPEFQGRIRMTRDTFIDTVYVDLSGLTPADTAYYYCARGIDGNSYPFHFWGHGTRVTVFSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 203 aa, Homo sapiens
RFALTQPASVSGSPGQTITITCAGGSVSWFHFPPGKTPRLIIYESSKRPSGVSPRFSGSQSGSTASLIISGLQSDDEGTYFCSILEFFGRGTLLTVLSQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 77 - DNA, 1374 bp, Homo sapiens
  source 1..1374: mol_type = other DNA
gcgggactgatgcagtctggggctgtgatgaagaattcgggggcctcagtgagggtctcttgtcaggctgatggatacgacttcattgactatgtcattcactggtttcgacaaagacgtggagaaggtcttgagtggctgggatggatgaatccctcgggaggcggcacaaactatccgcgaccatttcagggcaaagtcaccatgaccagggacacgtccaccgagacagcctatttagatgtcagaggacttacatatgacgacacggccgtctattattgtgtgagagacagggccaacggttcgggaagaagacgttttgagtcggtgaattggttcctggatctgtggggccgcggcacccaaataacagtcgtctcgccctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 78 - DNA, 615 bp, Homo sapiens
  source 1..615: mol_type = other DNA
cagtctgccctaactcagcctcgctcagtgtccgcatctcctggtcagtcagtcaccatctcctgcactggaacacacaattatgtgtcctggtgtcaacagaaaccgggccaagcccccaaattattaatttacgatttcaataaacggccctcaggggtctctgatcgcttctctggctccacgtctggcaacacggcctccctgaccatctctggactccaggctgacgatgagggtcattatttttgttgggcgtttgaaaatatcggcggagggaccaagctgaccgtcctgcgtcagcccaaggctgccccctcggtcactctgttcccgccctcctctgaggagcttcaagccaacaaggccacactggtgtgtctcataagtgacttctacccgggagccgtgacagtggcttggaaagcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 79 - DNA, 1371 bp, Homo sapiens
  source 1..1371: mol_type = other DNA
gcggacttggtgcagtctggggctgtggtgaagaagcctggggactcagtgaggatctcctgtgaggctcaaggatatagatttcctgactacatcattcactggattcgacgggcccctggacaaggccctgaatggatgggatggatgaatccaatgggcggacaagtaaatattccatggaaatttcagggtagggtctccatgacccgggacacgtccatcgaaacagcatttctggacttaagaggactaaagtctgacgacacggccgtctattattgcgtgagagatcgcagtaatggatcgggaaagcgattcgagtcctccaattggttcctcgatctgtggggccgtgggactgcggtcacaattcaatcatcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 80 - DNA, 609 bp, Homo sapiens
  source 1..609: mol_type = other DNA
gccctgactcagcctcgctcagtgtccgcatctcctgggcagtccgtcaccatttcctgcactggaacccacaatttggtctcttggtgtcaacatcagccaggcagagcccccaaattattaatttatgacttcaataagaggccctcaggggtccctgatcgcttctctggctccgggtctggcggcacggcctccctgaccatcactggactccaggatgacgatgacgccgaatatttttgttgggcgtatgaagcttttggcggagggaccaagttgaccgttcttcgtcagcccaaggctgccccctcggtcactctgttcccgccctcctctgaggagcttcaagccaacaaggccacactggtgtgtctcataagtgacttctacccgggagccgtgacagtggcttggaaagcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 81 - DNA, 1371 bp, Homo sapiens
  source 1..1371: mol_type = other DNA
gcggacttggtgcagtctggggctgtggtgaagaagcctggggactcagtgaggatctcctgtgaggctcaaggatacaaatttcctgactacatcattcactggattcgacgggcccctggacaaggccttgagtggatggggtggattaatccaatgggcggacaagtaaacattccatggcagtttcagggcagggtctccatgacccgggacacgtccatcgaaacagcatttctggacttaagaggactaaagtctgacgacacggccctctattattgcgtgagagatcgaagtaatggatcgggaaggcgattcgagtcctccaattggttcctcgatctgtggggccgcggcactgcggtcactgttcattcaccctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 82 - DNA, 615 bp, Homo sapiens
  source 1..615: mol_type = other DNA
cagtctgccctgactcagcctcgctcagtgtccgcatctcctgggcagtccgtcaccatttcctgcactggaacccacaatttggtctcttggtgtcaacatcacccaggcagagcccccaaattattaatttatgacttcaataagaggccctcaggggtccctgatcgcttctctggctccgggtctggcggcacggcctccctgaccatcagtggactccaggatgacgatgacgccgaatatttttgttgggcgtatgaagcttttggcggagggaccaagttgaccgtacttcgtcagcccaaggctgccccctcggtcactctgttcccgccctcctctgaggagcttcaagccaacaaggccacactggtgtgtctcataagtgacttctacccgggagccgtgacagtggcttggaaagcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 83 - DNA, 1374 bp, Homo sapiens
  source 1..1374: mol_type = other DNA
tcggcggaattggtgcaatctggggctgtggtgaagaagcctgggacctccgtgaaggtctcttgtcaggcttatggatacacttttaccgactaccttattcattggcttcgacaggcccctggacaaggacttgaatggatgggatggatgaatcctgtttatggacaagtaaattatgcccaaaactttcagggcagggtctccatgaccagggacatttacagggaaacagcatttctagaggtgcgcgacctgaagactgacgacacaggcacttattattgtgtgagagacacaggcgacggttcgcggagacactttgactccatcaattggtttctcgatctttggggccgcgggacatggataagggtcgccccagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 84 - DNA, 615 bp, Homo sapiens
  source 1..615: mol_type = other DNA
cagtgtgtcttgactcagcctcgctcagtgtccggatctcctggacaatcagtcaccatctcctgcactggaactcacaattatgtctcctggtgtcaacaccacccaggcaacgcccccaaattattactttatgatttcgacaagcggccctcaggaatctctgatcgcttctctggctctaggtctggcaacacggcctccctgaccatctctggcctccagcctgaggatgaggccgattacttttgttgggcctttgaagcctttggcggagggaccaaggtgctcgtccttcgtcagcccaaggctgccccctcggtcactctgttcccgccctcctctgaggagcttcaagccaacaaggccacactggtgtgtctcataagtgacttctacccgggagccgtgacagtggcttggaaagcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 85 - DNA, 1371 bp, Homo sapiens
  source 1..1371: mol_type = other DNA
gcggacttggtgcagtctggggctgtgatgaagaagcctggggactcagtgagaatctcctgtgaggctcgaggatacacattcactgactacgtcattcactggattcgacgggcccctggacaaggccttgaatggatggggtggattgatccaccttatggacaagtaaatattccatggaattttcagggcagggtctccatgacccgggacacgtccatcgaaacagcatttctggacttaagaggtctaaagtctgacgacacgggcctctattattgcgtgagagatcgaagtaatggatggggaaagcgattcgagtcctccaattggttcctcgatctgtggggccgcggcactgtggtcactgttcactcaccctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 86 - DNA, 615 bp, Homo sapiens
  source 1..615: mol_type = other DNA
cagtctgccctgactcagcctcgctcagtgtccgcatctcctgggcagtccgtcaccatttcctgcactggaacccacaatttggtctcttggtgtcaacatcacccaggcagagcccccaagttattaatttatgacttcaataagaggccctcaggggtccctgatcgcttctctggctccgggtctggcggcacggcctccctaaccatcagtggactccaggatgacgatgacgccgaatatttttgttgggcatatgaagctttcggcggagggaccaagttgactgtacttcgtcagcccaaggctgccccctcggtcactctgttcccgccctcctctgaggagcttcaagccaacaaggccacactggtgtgtctcataagtgacttctacccgggagccgtgacagtggcttggaaagcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 87 - DNA, 1371 bp, Homo sapiens
  source 1..1371: mol_type = other DNA
gcggacttggtgcagtctggggctgtggtgaagaagcctggggactcagtgagaatctcctgtgaggctcaaggatacacattcactgactacgtcattcactggattcgacgggcccctggacaaggccttgaatggatggggtggattaatccaggttatggacaagtaaatattccatggaactttcagggcagggtctccatgacccgagacacgtccatcgaaacagcatttctggacttaagaggtctaaagtctgacgacacgggcctctattattgcgtgagagatcgaagtaatggatggggaaagcgattcgagtcctccaattggttcctcgatctgtggggccgcggcactgtggtcactgttcactcaccctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 88 - DNA, 615 bp, Homo sapiens
  source 1..615: mol_type = other DNA
cagtctgccctgactcagcctcgctcaatgtccgcatctcctgggcagtccgtcaccatttcctgcactggaacccacaatttggtctcttggtgtcaacatcacccaggcagaccccccaaattattaatttatgacttcaataagaggccctcaggggtccctgatcgcttctctggctccgggtctggcggcacggcctccctgaccatcagtggactccaggatgacgatgacgccgaatacatttgttgggcatatgaagctttcggcggagggaccaagttgaccgtacttcgtcagcccaaggctgccccctcggtcactctgttcccgccctcctctgaggagcttcaagccaacaaggccacactggtgtgtctcataagtgacttctacccgggagccgtgacagtggcttggaaagcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 89 - DNA, 1371 bp, Homo sapiens
  source 1..1371: mol_type = other DNA
gcggacttggtgcagtctggggctgtggtgaaaaatgctggggcctcagtgagggtctcctgtgaggcttatggatacacattcgtggactacttcattcattgggtccgacaggcccctggacaaggctttgaatggatgggatacatggatcccttgaacgggcgcccaaacattgcgcgaaaatttcagggcaggctctccctgagtcgagataggtccagcgaaacttcatttctggacttaagtggactgaggtctgacgactcggccgtctattattgtgtgagagacaagagtaatggatcgggcagacggtttgactcgtctaattggtttctcgatctgtggggccgtggaacccgggtcagtattttctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 90 - DNA, 615 bp, Homo sapiens
  source 1..615: mol_type = other DNA
cagtctgccctgactcagcctcgctcagtgtccgcaactcctggacagtcagtcaccatctcctgcactggaacccacaattatgtctcttggtgtcaacaacatccaggcagagcccccaaattactaatttatgacttcaataagaggccctcaggggtcccggatcgcttctctggctccggatctggcggcacggcctccctaaccatcactggactccaggatgacgatgaagcggactatttttgttgggcctatgatgcttttggcggagggaccaagttgaccgtcctgcgtcagcccaaggctgccccctcggtcactctgttcccgccctcctctgaggagcttcaagccaacaaggccacactggtgtgtctcataagtgacttctacccgggagccgtgacagtggcttggaaagcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 91 - DNA, 1371 bp, Homo sapiens
  source 1..1371: mol_type = other DNA
gcggacttggtgcagtctggggctgtggtgaagaagcctggggactcagtgagggtctcctgtgaggcttatggatacacattcagtgactacatcattcattggattcgacgggcccctggacgaggccttgaatggatgggatggatgaatccgatgggcggacaagtgaatattccgtggaactttcaggggagagtctccatgacccgggacacgtccatcgaaacagcatttctggacttaagaggactgaggtctgacgacacggccgtctattactgtgtgagagatcgcagcaatggatcgggcaagcgatttgagtcctccaattggttcctcgatctgtggggccgcgggaccgcggtcactatttcctcaccctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 92 - DNA, 615 bp, Homo sapiens
  source 1..615: mol_type = other DNA
cagtctgccctgactcagcctcgctcagtgtccgcagctcctggacagtccgtcaccatttcctgcactggaacccacaatttggtttcttggtgtcaacatcacccaggcagagcccccaagttattaatttatgacttcaataagagaccctcaggtgtccctgatcgtttctctggctccgggtctggcggcacggcctccctaaccatcactggactccaggatgacgatgaggctgaatatttttgttgggcgtatgaagtttttggcggagggaccaagttgaccgtgcttcgtcagcccaaggctgccccctcggtcactctgttcccgccctcctctgaggagcttcaagccaacaaggccacactggtgtgtctcataagtgacttctacccgggagccgtgacagtggcttggaaagcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 93 - DNA, 1371 bp, Homo sapiens
  source 1..1371: mol_type = other DNA
gcggacttggtgcagtctggggctgtggtgaagacgcctggggcctcagtgagggtctcctgtgaggcttatggatacacattcattgactacatcattcattgggtccgacaggcccctggacaaggttttgaatggctgggatacatcgatcctatgaacgggcgcccaaacattgcgcgaaaatttcagggcaggctctccctgagccgggatacgtccatcgaaacatcatttctggacttaagtggactgaggtctgacgactcggccgtctattattgtgtgagagacaagagtaatggatcgggcaaacgatttgactcctctaattggtttctcgatctgtggggccgtggaacgcgggtcagcatttcttcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 94 - DNA, 615 bp, Homo sapiens
  source 1..615: mol_type = other DNA
cagtctgccctgactcagcctcgctcagtgtccgcagctcctggacagtcagtcaccatctcctgcactggaacccacaattatgtctcttggtgtcaacaacatccaggcagagcccccaaattactaatttatgacttcaataagaggccctcaggggtcccggatcgcttctctggctccggatctggcggcacggcctccctaaccatcactagactccaggatgacgatgacgctgactatttttgttgggcgtatgatgcttttggcggagggaccaagttgaccgtcctgcgtcagcccaaggctgccccctcggtcactctgttcccgccctcctctgaggagcttcaagccaacaaggccacactggtgtgtctcataagtgacttctacccgggagccgtgacagtggcttggaaagcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 95 - DNA, 1371 bp, Homo sapiens
  source 1..1371: mol_type = other DNA
gcggacttggtgcagtctggggctgtggtgaagaagcctggggcctcagtgagggtctcctgtgaggcttatggatacacattcactgactacatcattcattggattcgacaggcccctggacaaggccttgaatggatgggatggatgaatcctatgggcgggcgcacaaatattccgtggaaatttcagggcagggtctccatgacccgggacacgtccatcgaaacagcatttctggacttaagtggactaacgtctgacgacacggccgtctattattgcgtgagagacaagagtaatggatcgggcaaacgatttgactcctctaattggttcctcgatctgtggggccgcggaaccccggtcactatttcctcaccctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 96 - DNA, 615 bp, Homo sapiens
  source 1..615: mol_type = other DNA
cagtctgccctgactcagcctcgctcagtgtccgcagctcctggacagtccgtcaccatttcctgcactggaacccacaatttggtctcttggtgtcaacaacacccaggcagagcccccaaattattaatttatgacttcaataagaggccctcaggggtccctgatcgcttctctggctccgggtctggcggcacggcctccctaagtatcactggactccaggatgacgatgaagctgaatatttttgttgggcgtatgaagcttttggcggagggaccaagttgaccgtccttcgtcagcccaaggctgccccctcggtcactctgttcccgccctcctctgaggagcttcaagccaacaaggccacactggtgtgtctcataagtgacttctacccgggagccgtgacagtggcttggaaagcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 97 - DNA, 1371 bp, Homo sapiens
  source 1..1371: mol_type = other DNA
gcggacttggtgcagtctggggctgtggtgaagaagcctggggcctcagtgcgggtctcctgtgaggcttatggatatacattcgttgactacctcattcattgggtccgacaggcccccggacaaggttttgaatggatgggatacatggatcctatgaacgggcgcccaaatattgcgcgaaaatttcagggcaggctctccctgagccgagatacgtccatcgaaacatcatttctggacttaagtggactgaggtctgacgactcggccgtctattattgtgtgagagacaagagtggtggatcgggcaaactatttgactcctctaattggtttctcgatctgtggggccgtggaacccgggtcagcatttcttcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 98 - DNA, 615 bp, Homo sapiens
  source 1..615: mol_type = other DNA
cagtctgccctgactcagcctcgctcagtgtccgcagctcctggacagtcagtcaccatctcctgcaccggaactcacaattatgtctcttggtgtcaacaacatccaggcagagcccccaaattactaatttatgacttcaataagaggccctcaggggtcccggatcgcttctctggctccggatctggcggcacggcctccctaaccatcactggactccaggatgacgatgacgctgattatttttgttgggcgtatgatgcttttggcggagggaccaagttgaccgtcctgcgtcagcccaaggctgccccctcggtcactctgttcccgccctcctctgaggagcttcaagccaacaaggccacactggtgtgtctcataagtgacttctacccgggagccgtgacagtggcttggaaagcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 99 - DNA, 1371 bp, Homo sapiens
  source 1..1371: mol_type = other DNA
gcggacttggtgcagtctggggctgtggtgaagaagcctggggactcagtgagaatctcctgtgaggctcaaggatacacattcactgactacgtcattcactggattcgacgggcccctggacaaggccttgaatggatggggtggattaatccaggttatggacaagtaaatattccatggaactttcagggcagggtctccatgacccgagacacgtccatcgaaacagcttttctggagttaagaggtctaaagtctgacgacacgggcctctattattgcgtgagagatcgaagtaatggatggggaaagcgattcgagtcctccaattggttcctcgatctgtggggccgcggcactgtgattactgttcactcaccctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 100 - DNA, 615 bp, Homo sapiens
  source 1..615: mol_type = other DNA
cagtctgccctgactcagcctcgctcaatgtccgcatctcctgggcagtccgtcaccatttcctgcactggaacccacaatttggtctcttggtgtcaacatcacccaggcagaccccccaaattattaatttatgacttcaataagaggccctcaggggtccctgatcgcttctctggctccgggtctggcggcacggcctccctgaccatcactggactccaggatgacgatgacgccgaatacatttgttgggcatatgaagctttcggcggagggaccaagttgaccatacttcgtcagcccaaggctgccccctcggtcactctgttcccgccctcctctgaggagcttcaagccaacaaggccacactggtgtgtctcataagtgacttctacccgggagccgtgacagtggcttggaaagcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 101 - DNA, 1371 bp, Homo sapiens
  source 1..1371: mol_type = other DNA
gcggacttggtgcagtctggggctgtggtgaagaagcctggggactcagtgagaatctcctgtgaggctcagggatatacattcaccgactacgtcattcattggattcgacgggcccctggacaaggccttgaatggatggggtggatggatccaagttatggacaagtcaatattccacggaactttcagggcagggtctccatgacccgggacacgttcagggaaacagcatatctggaattaagaggtctacagtctgacgacaagggcctctattattgtgtgagagatcgaagtcacggatcgggaaggcaattcgagtcctccaactggttcctcgatctgtggggccgcggcactgtggtcaatgttcagtcaccctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 102 - DNA, 615 bp, Homo sapiens
  source 1..615: mol_type = other DNA
cagtctgccctgactcagcctcgctcagtgtccgcatctcctgggcagtccgtcaccatttcctgcactggaacccacaatttggtctcttggtgtcaacatcacccaggcagacctcccaaattattaatttatgacttcaataagagggcttcaggggtccctgatcgcttctctggctccgggtctggcggcacggcctccctgaccattagtggactccaggatgacgatgacgccgaatatttttgttgggcatatgaagctttcggcggagggaccaagttgaccgtacttcgtcagcccaaggctgccccctcggtcactctgttcccgccctcctctgaggagcttcaagccaacaaggccacactggtgtgtctcataagtgacttctacccgggagccgtgacagtggcttggaaagcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 103 - DNA, 1371 bp, Homo sapiens
  source 1..1371: mol_type = other DNA
gcggacttggtgcagtctggggctgtggtgaagaagcctggggactcactgagaatctcctgtgaggctcaaggatacacattcactgactacgtcattcactggattcgacgggcccctggacaaggccttgaatggatgggatggatggatccaagttttggacaaatgaacattccacggaactttcagggcagggtctccatgacccgtgacatgtacatcgaaacagcatttctggacttaagaggtctaaagtctgacgacacgggcctctattattgcgtgagagatcgaagtcatggatcgggaaggctattcgagtcctccaattggttcctcgatctgtggggccgcggcactgtggtcactgttcagtcaccctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 104 - DNA, 615 bp, Homo sapiens; identical to SEQ ID NO: 102
  source 1..615: mol_type = other DNA

SEQ ID NO: 105 - DNA, 1371 bp, Homo sapiens
  source 1..1371: mol_type = other DNA
gcggacttggtgcagtctggggctgtggtgaagaagcctggggactcagtgagaatctcctgtgaggctcaaggatacagattcactgactacgtcattcattggattcgacgggcccctggacaaggccttgaatggatggggttgatggatccaagttttggacgaatgaatattccacggaaatttcagggcagggtctccatgacccgggacacgtccatggaaacagcatttctggacttcagaggtctaaattttgacgacacgggcctctattattgcgtgagagatcgaagtcatggatcgggaagactattcgagtcctccaattggttcctcgatctgtggggccgcggcactgtggtcactgttcagtcaccctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 106 - DNA, 615 bp, Homo sapiens
  source 1..615: mol_type = other DNA
cagtctgccctgactcagcctcgctcagtgtccgcatctcctgggcagtccgtcaccatttcctgcactggaacccacaatttggtctcttggtgtcaacatcacccaggcagacctcccaaattattaatttatgacttcaataagagggcatcaggggtccctgatcgcttctctggctccgggtctggcggcacggcctccctgaccatcagtggactccaagatgacgatgacgccgaatatttttgttgggcatatgaagctttcggcggagggaccaagttgaccgtacttcgtcagcccaaggctgccccctcggtcactctgttcccgccctcctctgaggagcttcaagccaacaaggccacactggtgtgtctcataagtgacttctacccgggagccgtgacagtggcttggaaagcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 107 - DNA, 1371 bp, Homo sapiens
  source 1..1371: mol_type = other DNA
ccggacttggtgcagtctggggctgtggtgaagaagcctggggactcagtgagaatttcctgtgaggctcaaggatacacattcattgactacgtcattcactggattcgacgggcccctggacaaggccttgaatggatggggttgatggatccaacttatggacgaatgaatattccacggaagtttcagggcagggtctccatgacccgggacacgtccatcgaaacagcatttctggacttaagaggtctaaaatctgacgacacgggcctctattattgcgtgagagatcgaagtcatggatcgggaaggctattcgagtcctccaactggttcctggatctgtggggccgcggcactgtggtcactgttcagtcaccctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 108 - DNA, 615 bp, Homo sapiens
  source 1..615: mol_type = other DNA
cagtctgccctgactcagcctcgctcagtgtccgcatctcctgggcagtccgtcaccatttcctgcactggaacccacaatttggtctcttggtgtcaacatcacccaggcagacctcccaaattattaatttatgacttcaataagagggcttcaggggtccctgatcgcttctctggctccgggtctggcggcacggcctccctgaccatcagtggactccaagatgacgatgacgccgaatatttttgttgggcatatgaagctttcggcggagggaccaagttgaccgtacttcgtcagcccaaggctgccccctcggtcactctgttcccgccctcctctgaggagcttcaagccaacaaggccacactggtgtgtctcataagtgacttctacccgggagccgtgacagtggcttggaaagcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 109 - DNA, 1371 bp, Homo sapiens
  source 1..1371: mol_type = other DNA
gcggacttggtgcagtctggggctgtggtgaagaagcctggggactcagtgaggatctcctgtgaggctcaaggatacagatttcttgactacatcattcactggattcgacgggcccctggacaaggccttgaatggatgggatggatgaatccaatgggcggacaagtaaacattccatggaactttcagggtagggtctccatgacccgggacacgtccatcgaaacagcatttctggacttaagaggactaaagtctgacgacacggccgtctattattgcgtgagagatcgcagtaatggatcgggaaagcgattcgagtcctccaattggttcctcgatctgtggggccgcgggactgcggtcactattcattcaccctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 110 - DNA, 615 bp, Homo sapiens
  source 1..615: mol_type = other DNA
cagtctgccctgactcagcctcgctcagtgtccgcatctcctgggcagtccgtcaccatttcctgcactggaacccacaatttggtctcttggtgtcaacatcacccaggcagagcccccaaattattaatttatgacttcaataagaggccctcaggggtccctgatcgcttctctggctccgggtctggcggcacggcctccctgaccatcactggactccaggatgacgatgacgccgaatatttttgttgggcgtatgaagcttttggcggagggaccaagttgaccgtacttcgtcagcccaaggctgccccctcggtcactctgttcccgccctcctctgaggagcttcaagccaacaaggccacactggtgtgtctcataagtgacttctacccgggagccgtgacagtggcttggaaagcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 111 - DNA, 1350 bp, Homo sapiens
  source 1..1350: mol_type = other DNA
cacgtccaattggtgcagtctggaggtggggtgaagaagattggggccgctgtgaggatctcctgcgaggtgactggatataaattcatggaccaactcataaactgggtgcggcaggcccccggtcagggccttgagtggatgggatggatgaatccaacatatggacaagtaaattattcatggagatttgaaggaagggtcaccatgaccagggacatggacaccgagacggccttcatggagttgagaggactgagagtggacgacacggccgtctattattgcgcgagggggccctctggggaaaattatccttttcactattggggccagggtgtccgagtggtcgtctcgtcaccctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 112 - DNA, 618 bp, Homo sapiens
  source 1..618: mol_type = other DNA
gcatctgccctgactcagcctgcctccatgtctgcgtcccctggacagtcggtaaccatctcgtgctctggaaccagacacataatctctgcttggttccaacaatatccaggcaaaccacccaaactcataatttttgacgacgataagcgtccctctggagttcctagtcgcttctctgcctccaggcctggcgacacggcctccctgacaatctctaatgttcaacctgaggacgaggcgacgtacatttgcaatacatatgaattctttggcggagggaccagattgaccgtcctaagtcagcccaaggctgccccctcggtcactctgttcccaccctcctctgaggagcttcaagccaacaaggccacactggtgtgtctcgtaagtgacttctacccgggagccgtgacagtggcctggaaggcagatggcagccccgtcaaggtgggagtggagaccaccaaaccctccaaacaaagcaacaacaagtatgcggccagcagctacctgagcctgacgcccgagcagtggaagtcccacagaagctacagctgccgggtcacgcatgaagggagcaccgtggagaagacagtggcccctgcagaatgctct

SEQ ID NO: 113 - DNA, 1350 bp, Homo sapiens
  source 1..1350: mol_type = other DNA
cacattcagttgctacagtcggggcctcaagttaagaagtctggggacacagtgagaatctcctgtgagacctctggatataacttcgtcgactcccgtatccactgggtccgacagaccccggaaaaacgtctcagatggatgggctggatcaatcctctccaaggtggtgtgaattacgcgccggaatttcagggcagaatcaggatgaccagggacacatttatagacacagtttacgtggacctgagcggactgacaccggccgacacggcctattattactgcgcgcgagggatcgatggcaagtcttacccctttcatttctggggccacggaacccgggtcaccgtcttctcggcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 114 - DNA, 609 bp, Homo sapiens
  source 1..609: mol_type = other DNA
cgatttgccctgactcaacctgcctccgtgtctgggtctcctggacagacgatcaccataacctgcgctggaggcagcgtctcctggtttcatttccctccaggcaaaacccccagactcattatttatgagtcttctaagcgaccctctggggtctctcctcgattctctgggtcccagtctggcagcacggcctcccttataatttctggcctccagtctgatgacgaagggacatacttctgttctattcttgaatttttcggcagagggactcttgtcaccgtcctgagtcagcccaaggctgccccctcggtcactctgttcccaccctcctctgaggagcttcaagccaacaaggccacactggtgtgtctcgtaagtgacttctacccgggagccgtgacagtggcctggaaggcagatggcagccccgtcaaggtgggagtggagaccaccaaaccctccaaacaaagcaacaacaagtatgcggccagcagctacctgagcctgacgcccgagcagtggaagtcccacagaagctacagctgccgggtcacgcatgaagggagcaccgtggagaagacagtggcccctgcagaatgctct

SEQ ID NO: 115 - DNA, 1359 bp, Homo sapiens
  source 1..1359: mol_type = other DNA
caggtgcgcttggtgcagtctggggctggggcgaggaagactggggcctcaatgaaactttcctgctcgacctctggatacaccttcaccactcatcacggccacttcataaattgggtgcgacaggcccgtggacaagggcttgagtggatggggtggatgaatcccatgactgggcagatgaatattgaggggaaatttcagggcagagtcaccctcactcgagacatatacagtgacacggcttacatggaaatgaccagactgacaactggcgacacgggcacttattactgtgcgcgaggcgatttcggacagaattatccctttcattattggggccagggaagcctggtcatcgtctcctcggcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 116 - DNA, 618 bp, Homo sapiens
  source 1..618: mol_type = other DNA
ctgtctgccctgactcagcctgcctccgtgtctgggtctcctgggcagtcggtcaccatctcctgctctggaacgaaccgttaccttgtctcctggtatcaacaacaccctgacaaagcccccaaactcatcatttatgacgacaataagcggccctcaggaatttctgatcgcttctcagcctccaggcctgacgacacggcctccctgacaatctctggactccagactggggacgaggctacttattggtgtgcctcatatgaacgttttggcggcgggacgaggctgaccgtccttagtcagcccaaggctgccccctcggtcactctgttcccaccctcctctgaggagcttcaagccaacaaggccacactggtgtgtctcgtaagtgacttctacccgggagccgtgacagtggcctggaaggcagatggcagccccgtcaaggtgggagtggagaccaccaaaccctccaaacaaagcaacaacaagtatgcggccagcagctacctgagcctgacgcccgagcagtggaagtcccacagaagctacagctgccgggtcacgcatgaagggagcaccgtggagaagacagtggcccctgcagaatgctct

SEQ ID NO: 117 - DNA, 1350 bp, Homo sapiens
  source 1..1350: mol_type = other DNA
cacgtccaattggtgcagtctggaggtggggtgaagaagattggggccgctgtgaggatctcctgcgaggtgtctggatacaacttcatggaccaattcataaattgggtgcgacaggcccccggtcagggccttgagtggatgggatggatgaacccaatatatggacaagtaaattattcatggagatttcaaggaagggtcaccatgaccagggacatgtacaccgacacggccttcatggagttgagaggactgagagtggacgacacggccgtctattattgcgcgagggggccctctggggaaaattatccttttcactattggggccagggtgtccgagtggtcgtctcgtcaccctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 118 - DNA, 609 bp, Homo sapiens
  source 1..609: mol_type = other DNA
ctgactcagcctgcctccatgtctgcgtcccctggacagtcggtaaccatctcgtgctctggaaccagacacataatctctgcttggttccaacaatatccaggcaaaccacccaaactcataatttttgacgacgataagcgtccctctggagttcctagtcgcttctctgcctccaggcctggcgacacggcctccctgacaatctctaatgttcaacctgaggacgaggcgacatacatttgcaatacatatgaattctttggcggagggaccaaattgaccgtcctaagtcagcccaaggctgccccctcggtcactctgttcccaccctcctctgaggagcttcaagccaacaaggccacactggtgtgtctcgtaagtgacttctacccgggagccgtgacagtggcctggaaggcagatggcagccccgtcaaggtgggagtggagaccaccaaaccctccaaacaaagcaacaacaagtatgcggccagcagctacctgagcctgacgcccgagcagtggaagtcccacagaagctacagctgccgggtcacgcatgaagggagcaccgtggagaagacagtggcccctgcagaatgctct

SEQ ID NO: 119 - DNA, 1350 bp, Homo sapiens
  source 1..1350: mol_type = other DNA
cacattcagttgctacagtcggggcctcaagttaagaagtctggggacacagtgagaatctcctgtgagacctctggatataacttcgtcgactcccgtatccactgggtccgacagaccccggaaaaacgtctcagatggatgggctggatcaatcctctccacggtggtgtgaattacgcgccggaatttcagggcagaatcaggatgaccagggacacatttatagacacagtttacgtggacctgagcggactgacaccggccgacacggcctattattactgcgcgcgagggatcgatggcaagtcttacccctttcatttctggggccacggaacccgggtcaccgtcttctcggcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 120 - DNA, 609 bp, Homo sapiens
  source 1..609: mol_type = other DNA
cgatttgccctgactcaacctgcctccgtgtctgggtctcctggacagacgatcaccataacctgcgctggaggcagcgtctcctggtttcatttccctccaggcaaaacccccagactcattatttatgagtcttctaagcgaccctctggggtctctcctcgattctctgggtcccagtctggcagcacggcctccctcataatttctggcctccagtctgatgacgaagggacatacttctgttctattcttgaatttttcggcagagggactcttgtcaccgtcctgagtcagcccaaggctgccccctcggtcactctgttcccaccctcctctgaggagcttcaagccaacaaggccacactggtgtgtctcgtaagtgacttctacccgggagccgtgacagtggcctggaaggcagatggcagccccgtcaaggtgggagtggagaccaccaaaccctccaaacaaagcaacaacaagtatgcggccagcagctacctgagcctgacgcccgagcagtggaagtcccacagaagctacagctgccgggtcacgcatgaagggagcaccgtggagaagacagtggcccctgcagaatgctct

SEQ ID NO: 121 - DNA, 1365 bp, Homo sapiens
  source 1..1365: mol_type = other DNA
cgtgttacattacaacaatctggggctatagtgaggcagcctggggcctcagtgaccgtctcctgcgagacttctggatatactttcaccaagtatttcatctactgggtgcgacaggcccctggacagggtcttgagtggctgggcagaatacacccccgaaccggtgccgtgaagtatgcaccgagatttcagggtagactgtccatgaccagagactggtcactcgacacagcctacctcggattgaccggactgacactcggcgacacggctctatatttctgtgcgaggggggcctttgaggcagattcatatgggtcaagttatccctttcaccactggggccagggaaccctagtcaccgtctccgcggcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 122 - DNA, 621 bp, Homo sapiens
  source 1..621: mol_type = other DNA
tcctgggccctgactcaacccgcctccgtgtctgcgtctcctgggcagtcggtcaccatgtcctgcactggattcggaaattataaccctgactcctggtaccaacaatacccaggcaaagcccccaaactcatcatttatgaagacaataaaagaccctcgggggtctctgatcgcttctctgcctccagacttggcagcacgtcttccctgacaatctctaacgtccaggctgcggacgacgcccattatgtctgcgcctcctttgaatttttcggcggagggaccaagctgaccgtcctgagtcagcccaaggctgccccctcggtcactctgttcccaccctcctctgaggagcttcaagccaacaaggccacactggtgtgtctcgtaagtgacttctacccgggagccgtgacagtggcctggaaggcagatggcagccccgtcaaggtgggagtggagaccaccaaaccctccaaacaaagcaacaacaagtatgcggccagcagctacctgagcctgacgcccgagcagtggaagtcccacagaagctacagctgccgggtcacgcatgaagggagcaccgtggagaagacagtggcccctgcagaatgctct

SEQ ID NO: 123 - DNA, 1365 bp, Homo sapiens
  source 1..1365: mol_type = other DNA
cgtgtgacattacaacaatctggggctacagtgaagcagcctggggcctcagtgaccgtctcctgcgagacttctggatacactttcaccaagtataccattcactgggtgcgacaggcccctggacagggtcttcagtgggtgggcagaatacacccccgaaccggtgccgtgaagtatgcaccgatatttcagggtaaagtgtccatgagtcgagacttgtcacgcgacacagcctacctcggattgaccagactgacgctcgccgacacggctctatttttctgtgcgaggggggcctttgaggcagatttaagtgggccaacttacccctttcaccactggggccaaggaaccctagtcatcgtctccgcggcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 124 - DNA, 621 bp, Homo sapiens
  source 1..621: mol_type = other DNA
tcctgggccctgactcaacccgcctccgtgtctgcgtctcctgggcagtcggtcaccatgtcctgcactggattcggaaattataaccctgactcctggtaccaacaatacccaggcaaagcccccaaactcatcatttatgaggacaataaaagaccctcgggagtctctaatcgcttctctgcctccagacttggcagcacgtcttccctgacaatctctaacgtccaggccgctgacgacgcccattatgtctgcgcctcctttgaatttttcggcggagggaccaagctgatcgtcctgagtcagcccaaggctgccccctcggtcactctgttcccaccctcctctgaggagcttcaagccaacaaggccacactggtgtgtctcgtaagtgacttctacccgggagccgtgacagtggcctggaaggcagatggcagccccgtcaaggtgggagtggagaccaccaaaccctccaaacaaagcaacaacaagtatgcggccagcagctacctgagcctgacgcccgagcagtggaagtcccacagaagctacagctgccgggtcacgcatgaagggagcaccgtggagaagacagtggcccctgcagaatgctct

SEQ ID NO: 125 - DNA, 1350 bp, Homo sapiens
  source 1..1350: mol_type = other DNA
aacgtgcagttgatgcagtctgggactgaggtgaagaagtctggggcctcggtgacaatctcttgtgagaccgctggattcaacttcatcgactccgtcatacactggctgcgccaggcccctggaggaggatttcagtggatggggtggatcaagcctcttagaggtgccgtcaattatccacagtttttgcagggcagggtctccatgacccgggacttgtccaccgacacggtgtacatggtcttgaatggactgacacctgacgacacaggcctttattactgcgcgaaaggggcctttagagggggttctccctttggcttctggggccagggaactctgctcaccgtctccccagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 126 - DNA, 606 bp, Homo sapiens
  source 1..606: mol_type = other DNA
cagtctgccctgtctcagcctgtctccgtgtctgggtctcctggagagtcgatcaccatttcctgtactggagccaccacctggtatcaacaactcccaggcagaccccccaaactcatcatttatgacgtcactaaccggccctcaggcatttctagtcgtttctctggctccacgtctggccacacggcctccctgacaatctccggtctccaggttgacgacgagggtctgtatcactgcgcctcacgtgaatttttcggcggagggaccaagctgaccgtcctgagtcagcccaaggctgccccctcggtcactctgttcccaccctcctctgaggagcttcaagccaacaaggccacactggtgtgtctcgtaagtgacttctacccgggagccgtgacagtggcctggaaggcagatggcagccccgtcaaggtgggagtggagaccaccaaaccctccaaacaaagcaacaacaagtatgcggccagcagctacctgagcctgacgcccgagcagtggaagtcccacagaagctacagctgccgggtcacgcatgaagggagcaccgtggagaagacagtggcccctgcagaatgctct

SEQ ID NO: 127 - DNA, 1350 bp, Homo sapiens
  source 1..1350: mol_type = other DNA
caggtgcgactggtgcagtctgggcctcaggtgaagaagactggggcctcagtgagggtctcctgcgaaacctctggatacacgttcacctcctacttcatccattggttacgactgggccccggagaggggcttcagtggatggggtggatcaaccctttacatggtgccgtgaattatgaaaacaaatttaggggcagggtcacaatcaccagggacacgtccacagacacagtgtatttggacatgagcagactgacccctgacgacacggccgtctatttctgcacaagaggaatcgttgctgatgggtggccctatggccactggggccagggaacccaagtcaccgtctccccggcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 128 - DNA, 609 bp, Homo sapiens
  source 1..609: mol_type = other DNA
tcctgggccctgactcagcctgcctccgtgtctgggtctcctggacagtcggtcgccatctcctgcgctggcggcagcgtctcctggtaccaggtgctcccaggcagagcccccaaactcatcatttatgagggcgctaagcgaccctcaggggtttctgctcgcttctctggctcccagtctggcaacacggcttacctgacaatttctgacctccagactgaggacgagggcatctacttctgctcttcacttcaattcttcggcggagggaccaaactgaccgtcctaagtcagcccaaggctgccccctcggtcactctgttcccaccctcctctgaggagcttcaagccaacaaggccacactggtgtgtctcgtaagtgacttctacccgggagccgtgacagtggcctggaaggcagatggcagccccgtcaaggtgggagtggagaccaccaaaccctccaaacaaagcaacaacaagtatgcggccagcagctacctgagcctgacgcccgagcagtggaagtcccacagaagctacagctgccgggtcacgcatgaagggagcaccgtggagaagacagtggcccctgcagaatgctct

SEQ ID NO: 129 - DNA, 1353 bp, Homo sapiens
  source 1..1353: mol_type = other DNA
caggtgcgacttcagcagtctggtgtcgtggtgaggaagcctggggcctcagtgagaatttcctgcgagacttctggattcaccttcatcgaccacattgtccattgggtgcggcgggcccctggacgaggctttgaatggatgggttggatcaagcctcttaggggtgccgtagattatgcaccccaacttcggggcaggatctccctgacgagggacatttacagtgaaaccgtctttatagacgtgagtcgactgacgtctggcgacacggcgatatacttttgttgtaaggccgccgcccctgaagaagcattcccccttcaatactggggccaggggacccaacttatcgtctcctcggcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 130 - DNA, 615 bp, Homo sapiens
  source 1..615: mol_type = other DNA
caggctgccctgactcagcccgcctccgtgtccggctctcctggacagtcggtcaccatttcctgcctttatgccaatgtagatatctgctggtatcaactacacccgggcagagcccccaaacttctaattgttgacaataataagcggccctcaggagtctctcctcgcttctctggctccaagtctggcaccacggcctccctgacaatctctggacttcaggctgacgacgaggctgaatatcactgctcttcaagaacattttttggcggggggaccaagttgaccgtcctgagtcagcccaaggctgccccctcggtcactctgttcccaccctcctctgaggagcttcaagccaacaaggccacactggtgtgtctcgtaagtgacttctacccgggagccgtgacagtggcctggaaggcagatggcagccccgtcaaggtgggagtggagaccaccaaaccctccaaacaaagcaacaacaagtatgcggccagcagctacctgagcctgacgcccgagcagtggaagtcccacagaagctacagctgccgggtcacgcatgaagggagcaccgtggagaagacagtggcccctgcagaatgctct

SEQ ID NO: 131 - DNA, 1353 bp, Homo sapiens
  source 1..1353: mol_type = other DNA
caggtgcgacttcagcagtctggtgtcgtggtgaggaagcctggggcctcagtgagactttcctgcgagacgtctggattcaaattcatcgaccacattgtcaactgggtgcggcgggcccctggacgaggctttgaatggatgggttggatcaagcctcttgggggtgtcgctgattatgcaccccaacatcggggcaggatctcactgacgagggacatttacactgaaaccgtctttatagacctgagtcgactgacgtctggcgacacggcgatttatttctgttgtaaggccgccgcccctgatgaagcattcccccttgaatactggggccaggggacccaacttatcgtctccccggcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 132 - DNA, 615 bp, Homo sapiens
  source 1..615: mol_type = other DNA
caggctgccctgactcagcccgcctccgtgtccggctctcctggacagtcggtcaccatttcctgcctttatgccaatgtagatatctgctggtatcaaatacagccgggcagattacccaaacttctgattgttgacaataataggcgaccctcaggagtctctcctcgcttctctggctccaagtctggcaccacggcctccctgacaatctctggacttcaggctgacgacgaggctgaatatcactgctcttcaacaacattttttggcggggggaccaagttgaccgtcctcagtcagcccaaggctgccccctcggtcactctgttcccaccctcctctgaggagcttcaagccaacaaggccacactggtgtgtctcgtaagtgacttctacccgggagccgtgacagtggcctggaaggcagatggcagccccgtcaaggtgggagtggagaccaccaaaccctccaaacaaagcaacaacaagtatgcggccagcagctacctgagcctgacgcccgagcagtggaagtcccacagaagctacagctgccgggtcacgcatgaagggagcaccgtggagaagacagtggcccctgcagaatgctct

SEQ ID NO: 133 - DNA, 1353 bp, Homo sapiens
  source 1..1353: mol_type = other DNA
caggtgcgacttgtgcagtctggtcccgtgatgagaaagcctggggcctcagtgagaatttcttgcgagacatctggattcgccttcttggaccacattgtccactgggtgcggcgggcccctggacgcggctttgaatggatgggttgggttaagaccattgggggtgtcgttgattatgcaccccaccttaggggcaggatctccgtgacgagagacgtctttagtgaaaccgtctttctggacttgagtcgactgacgtctggcgacacggcgatgtatttttgttctaaggccgccgcccctgacgaagccttcccccttgaattttggggccaggggacccaagtcatcgtctcctcggcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 134 - DNA, 615 bp, Homo sapiens
  source 1..615: mol_type = other DNA
caggctgccctgactcagcccgcctccgtgtccggctctcctggacagtcggtcaccatttcttgcctttatgccaatgtggatatctgctggtatcaacttcacccgggcagagcccccaaacttcttattcttgacaataataaacggccctcaggagtctctagtcgcttctccggttccaagtctggcaccacggcctccctaaccatctctgaccttcaggctgacgacgaggctgaatatcactgctcttcaacaacattttttggcggggggaccaggttgaccgtcctgagtcagcccaaggctgccccctcggtcactctgttcccaccctcctctgaggagcttcaagccaacaaggccacactggtgtgtctcgtaagtgacttctacccgggagccgtgacagtggcctggaaggcagatggcagccccgtcaaggtgggagtggagaccaccaaaccctccaaacaaagcaacaacaagtatgcggccagcagctacctgagcctgacgcccgagcagtggaagtcccacagaagctacagctgccgggtcacgcatgaagggagcaccgtggagaagacagtggcccctgcagaatgctct

SEQ ID NO: 135 - DNA, 1353 bp, Homo sapiens
  source 1..1353: mol_type = other DNA
caggtgcgacttgtgcagtctggtcccgtggtgagaaagcctgggacctcagtgagaatttcttgcgagacatctggattcgccttcttggaccacattgtccactgggtgcggcgggcccctggacgcggctttgaatggatgggttgggttaagaccattgggggtgtcgttgattatgcaccccaccttaggggcaggatctccgtgacgagggacgtatttagtgaaatcgtctttatggagttgagtcgactgacgtctggcgacacggcgatgtatttttgttctaaggccgccgcccctgacgaagccttcccccttgaattttggggccaggggacccaagtcatcgtctcctcggcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 136 - DNA, 615 bp, Homo sapiens
  source 1..615: mol_type = other DNA
caggctgccctgactcagcccgcctccgtgtccggctctcctggacagtcggtcaccatttcctgcctttatgccaatgtggatatctgctggtatcaacttcacccgggcagagcccccaaacttctaattgttgacaataataagcggccctcaggagtctctagtcgcttctctggttccaagtctggcaccacggcctccctaacaatctctgatcttcaggctgacgacgaggctgaatatcactgctcttcaacaacattttttggcggggggaccaggttgaccgtcctgagtcagcccaaggctgccccctcggtcactctgttcccaccctcctctgaggagcttcaagccaacaaggccacactggtgtgtctcgtaagtgacttctacccgggagccgtgacagtggcctggaaggcagatggcagccccgtcaaggtgggagtggagaccaccaaaccctccaaacaaagcaacaacaagtatgcggccagcagctacctgagcctgacgcccgagcagtggaagtcccacagaagctacagctgccgggtcacgcatgaagggagcaccgtggagaagacagtggcccctgcagaatgctct

SEQ ID NO: 137 - DNA, 1350 bp, Homo sapiens
  source 1..1350: mol_type = other DNA
caagtgcaactggtgcagtctggggctggggtgaagaagcctggggcctcagtgagggtctcctgcgagacatccggattcaagttcaccgagtactttatccactttttacgacaggcccctggacaagggcttgagtggatgggatggctcaaccctctcagaggtgccgtcaactatccacggaagtttcagggcagagtcactttgaccagggacatctacaccaccaccgtctacatgcaacttaacggtctgacccctgacgacacggccgtctactactgtgccagagcggtctttaatgaagctttcccctttgactactggggccagggaagcctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 138 - DNA, 612 bp, Homo sapiens
  source 1..612: mol_type = other DNA
tcctgggcccagactcagcctgcctccgtgtctgggtctcctggacagtcgatcaccatctcctgcgctggaatcgtcagtgatgcctggtaccagcaatacccaggcagaccccccagactcatcctttatgacggcgataagcggccctcaggggtttctcctcgtttttctgcctccagggccggcaagacggcctccctgacaatttctgggctgcaggctgacgacgaggcttattatcactgcgcgtcaagggaattttttggaggcgtgaccaagttgaccgtcctaagtcagcccaaggctgccccctcggtcactctgttcccaccctcctctgaggagcttcaagccaacaaggccacactggtgtgtctcgtaagtgacttctacccgggagccgtgacagtggcctggaaggcagatggcagccccgtcaaggtgggagtggagaccaccaaaccctccaaacaaagcaacaacaagtatgcggccagcagctacctgagcctgacgcccgagcagtggaagtcccacagaagctacagctgccgggtcacgcatgaagggagcaccgtggagaagacagtggcccctgcagaatgctct

SEQ ID NO: 139 - DNA, 1353 bp, Homo sapiens
  source 1..1353: mol_type = other DNA
caggtgcgacttcagcagtctggtgtcgtggtgaggaagcctggggcctcagtgagactttcctgcgagacgtctggcttcaaattcatcgaccacattgtcaactgggtgcggcgggcccctggacgaggctttgaatggatgggttggatcaagcctcttgggggtgtcgctgattatgcaccccaacatcggggcaggatctcactgacgagggacatttacactgaaaccgtctttatagacctgagtcgactgacgtctggcgacacggcgatttatttttgttgtaaggccgccgcccctgatgaagcattcccccttgaatactggggccaggggacccaacttatcgtctccccggcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 140 - DNA, 615 bp, Homo sapiens
  source 1..615: mol_type = other DNA
caggctgccctgactcagcccgcctccgtgtccggctctcctggacagtcggtcaccatttcctgcctttatgccaatgtagatatctgctggtatcaaatacagccgggcagattacccaaacttctgattgttgacaatgataggcgaccctcaggagtctctcctcgcttctctggctccaagtctggcaccacggcctccctgacaatctctggacttcaggctgacgacgaggctgaatatcactgctcttcaacaacattttttggcggggggaccaagttgaccgtcctcagtcagcccaaggctgccccctcggtcactctgttcccaccctcctctgaggagcttcaagccaacaaggccacactggtgtgtctcgtaagtgacttctacccgggagccgtgacagtggcctggaaggcagatggcagccccgtcaaggtgggagtggagaccaccaaaccctccaaacaaagcaacaacaagtatgcggccagcagctacctgagcctgacgcccgagcagtggaagtcccacagaagctacagctgccgggtcacgcatgaagggagcaccgtggagaagacagtggcccctgcagaatgctct

SEQ ID NO: 141 - DNA, 1353 bp, Homo sapiens; identical to SEQ ID NO: 133
  source 1..1353: mol_type = other DNA

SEQ ID NO: 142 - DNA, 615 bp, Homo sapiens
  source 1..615: mol_type = other DNA
caggctgccctgactcagcccgcctccgtgtccggctctcctggacagtcggtcaccatttcttgcctttatgccaatgtggatatctgctggtatcaacttcacccgggcagagcccccaaacttctaattcttgacaataataaacggccctcaggagtctctagtcgcttctccggttccaagtctggcaccacggcctccctaaccatctctgaccttcaggctgacgacgaggctgaatatcactgctcttcaacaactttttttggcggggggaccaggttgaccgtcctgagtcagcccaaggctgccccctcggtcactctgttcccaccctcctctgaggagcttcaagccaacaaggccacactggtgtgtctcgtaagtgacttctacccgggagccgtgacagtggcctggaaggcagatggcagccccgtcaaggtgggagtggagaccaccaaaccctccaaacaaagcaacaacaagtatgcggccagcagctacctgagcctgacgcccgagcagtggaagtcccacagaagctacagctgccgggtcacgcatgaagggagcaccgtggagaagacagtggcccctgcagaatgctct

SEQ ID NO: 143 - DNA, 1350 bp, Homo sapiens
  source 1..1350: mol_type = other DNA
cacgtgcagttgaggcagtctgggactgaggcgaagaagtctggggcctcggtgacaatctcttgtgagaccgctggattcaacttcatcgactccgtcatacactggctgcgccaggcccctggtgggggatttcagtggatggggtggatcaagcctcttagaggtggcgtcaattatccacattatttgcagggcagaatctccatgacccgggacttgtccagtgacacggtttacatggtcttaaatagactgacacctgccgacacaggcctttattactgcgcgaaaggggcctttggggggagttctccctttggcttctggggccagggaactctgctcaccgtctccccagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 144 - DNA, 606 bp, Homo sapiens
  source 1..606: mol_type = other DNA
cagtctgccctgtctcagcctgtctccgtgtctgggtctcctggagagtcgatcaccatttcctgtactgaagccaccacctggtatcaacaactcccaggcaaaccccccaaactcatcatttatgacgtgaccaaccggccctcaggcatttcaagtcgtttctctggctccatgtctggtcgcacggcctccctgacaatctccggtctccaggttgacgacgagggtctctatcactgtgcctcacgtgaatttttcggcggggggaccaagctgaccgtcctgagtcagcccaaggctgccccctcggtcactctgttcccaccctcctctgaggagcttcaagccaacaaggccacactggtgtgtctcgtaagtgacttctacccgggagccgtgacagtggcctggaaggcagatggcagccccgtcaaggtgggagtggagaccaccaaaccctccaaacaaagcaacaacaagtatgcggccagcagctacctgagcctgacgcccgagcagtggaagtcccacagaagctacagctgccgggtcacgcatgaagggagcaccgtggagaagacagtggcccctgcagaatgctct

SEQ ID NO: 145 - DNA, 1350 bp, Homo sapiens
  source 1..1350: mol_type = other DNA
cacgtgcaattgatgcagtctgggactcaggcgaagaagtctggggcctcggtgacaatttcttgtgagaccgctggattcaagttcatcgactccgtcatacactggctgcgccaggcccctggagggggatttcagtggatggggtggatcaagcctcttggaggtgccgtcaactatccaccctatttgcagggcaggatctccttgacccgtgacttgtccaccgacacaatttacatggtcttgaatggactgacacctgccgacacaggcttttattactgcgccaaaggggcctttggggggggttctccctttggcttctggggccaggggactctgctcaccgtctccccagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 146 - DNA, 606 bp, Homo sapiens
  source 1..606: mol_type = other DNA
cagtctgccctgtctcagcctgtctccgtgtctgggtctcctggagactcgatcaccatttcttgttttggagccaccacctggtatcaacaactcccaggcagaccccccaaactcatcatttatgacgtgactaaccggccctcaggcatttcaggtcgtttctctggctccatgtctggtcaaaaggcctccctgacaatctccggtctccaggttgacgacgagggtctctatcactgcgcctcacgtgaatttttcggcggggggaccaaactgaccgtcctgagtcagcccaaggctgccccctcggtcactctgttcccaccctcctctgaggagcttcaagccaacaaggccacactggtgtgtctcgtaagtgacttctacccgggagccgtgacagtggcctggaaggcagatggcagccccgtcaaggtgggagtggagaccaccaaaccctccaaacaaagcaacaacaagtatgcggccagcagctacctgagcctgacgcccgagcagtggaagtcccacagaagctacagctgccgggtcacgcatgaagggagcaccgtggagaagacagtggcccctgcagaatgctct

SEQ ID NO: 147 - DNA, 1350 bp, Homo sapiens
  source 1..1350: mol_type = other DNA
cacgtgcagttgaggcagtctgggactgaggcgaagaagtctggggcctcggtgacaatctcttgtgagaccgctggattcaacttcatcgactccgtcatacactggctgcgccaggcccctggtgggggatttcagtggatggggtggatcaagcctcttagaggtggcgtcaattatccacattatttgcagggcagaatctccatgacccgggacttgtccagtgacacggtttacatggtcttaaatagactgacacctgacgacacaggcctttactactgcgcgaaaggggcctttggggggagttctccctttggcttctggggccagggaactctgctcaccgtctccccagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 148 - DNA, 606 bp, Homo sapiens
  source 1..606: mol_type = other DNA
cagtctgccctgtctcagcctgtctccgtgtctgggtctcctggagagtcgatcaccatttcctgtactgaagccaccacctggtatcaacaactcccagggagatcccccaaactcattatttatgacgtgaccaaccggccctcaggcatttcaagtcgtttctctggctccatgtctggtcgcacggcctccctgacaatctccggtctccaggttgacgacgagggtctctatcactgtgcctcacgtgaatttttcggcggggggaccaagctgaccgtcctcagtcagcccaaggctgccccctcggtcactctgttcccaccctcctctgaggagcttcaagccaacaaggccacactggtgtgtctcgtaagtgacttctacccgggagccgtgacagtggcctggaaggcagatggcagccccgtcaaggtgggagtggagaccaccaaaccctccaaacaaagcaacaacaagtatgcggccagcagctacctgagcctgacgcccgagcagtggaagtcccacagaagctacagctgccgggtcacgcatgaagggagcaccgtggagaagacagtggcccctgcagaatgctct

SEQ ID NO: 149 - DNA, 1365 bp, Homo sapiens
  source 1..1365: mol_type = other DNA
cgtgttacattacaacagtctggggctacagtgaggcagcctggggcctcagtcaccgtctcctgcgagacttctggattcaccttcatcaaatataccattcactgggtgcgacaggcccctggacagggtcttcagtgggtgggaagaatacacccccgaaccggtgccgtgaagtttgcaccgatatttcagggtaaattttccatgagtcgagacttgtcacgcgacacagcctacctcggattgaccagactgacactcgccgacacggctctatttttctgtgcgaggggggcctttgaggcagatttatatgggccaacttacccctttcaccactggggccaaggaacccaagtcaccgtctccgcggcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 150 - DNA, 621 bp, Homo sapiens
  source 1..621: mol_type = other DNA
tcctgggccctgactcaacccgcctccgtgtctgcgtctcctgggcagtcggtcaccatgtcctgcactggattcggaagttataatcctgactcctggtaccagcaatacccaggcaaagcccccaaactcatcatttatgatgacaataaaagaccctcgggggtctctgatcgcttctctgcctccagacttggcagcacatcttcactgacaatctctaacgtccaggccgctgacgacgcccattatgtctgcgcctcctttgagtttttcggcggggggaccaagctgaccgtcctgagtcagcccaaggctgccccctcggtcactctgttcccaccctcctctgaggagcttcaagccaacaaggccacactggtgtgtctcgtaagtgacttctacccgggagccgtgacagtggcctggaaggcagatggcagccccgtcaaggtgggagtggagaccaccaaaccctccaaacaaagcaacaacaagtatgcggccagcagctacctgagcctgacgcccgagcagtggaagtcccacagaagctacagctgccgggtcacgcatgaagggagcaccgtggagaagacagtggcccctgcagaatgctct

SEQ ID NO: 151 - DNA, 1350 bp, Homo sapiens
  source 1..1350: mol_type = other DNA
cacattcagttgctacagtcggggcctcaagttaagaagtctggggacacagtgagaatctcctgtgagacctctggatataatttcgtcgactcccttatccactgggtccgacagaccccggaaaaacgtctcagatggatgggctggatcaatcctctccaaggtggtgtgaattacgcgccggaatttcagggcagaatcaggatgaccagggacacgtttatagacacagtttacgtggacttgagcggactgacaccggccgacacggcctattattactgcgcgcgagggatcgatggcaattcttacccctttcatttctggggccacggaacccgggtcaccgtcttctcggcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 152 - DNA, 609 bp, Homo sapiens
  source 1..609: mol_type = other DNA
cgatttgccctgactcaacctgcctccgtgtctgggtctcctggacagacgatcaccataacctgcgctggaggcagcgtctcctggttccatttccctccaggcaaaacccccagactcattatttatgagtcttctaagagaccctcaggggtctctcctcgattctctgggtcccagtctggcagcacggcctccctaataatttctggcctccagtctgatgacgaagggacatacttctgttctattcttgaatttttcggcagagggactcttctcaccgtcctgagtcagcccaaggctgccccctcggtcactctgttcccaccctcctctgaggagcttcaagccaacaaggccacactggtgtgtctcgtaagtgacttctacccgggagccgtgacagtggcctggaaggcagatggcagccccgtcaaggtgggagtggagaccaccaaaccctccaaacaaagcaacaacaagtatgcggccagcagctacctgagcctgacgcccgagcagtggaagtcccacagaagctacagctgccgggtcacgcatgaagggagcaccgtggagaagacagtggcccctgcagaatgctct

SEQ ID NO: 153 - protein, 458 aa, synthetic construct
  REGION 1..458: note = antibody variant
AGLMQSGAVMKNSGASVRVSCQADGYDFIDYVIHWFRQRRGEGLEWLGWMNPSGGGTNYPRPFQGKVTMTRDTSTETAYLDVRGLTYDDTAVYYCVRDRANGSGRRRFESVNWFLDLWGRGTQITVVSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 154 - DNA, 1374 bp, synthetic construct
  misc_feature 1..1374: note = antibody variant
  source 1..1374: mol_type = other DNA
gcgggactgatgcagtctggggctgtgatgaagaattcgggggcctcagtgagggtctcttgtcaggctgatggatacgacttcattgactatgtcattcactggtttcgacaaagacgtggagaaggtcttgagtggctgggatggatgaatccctcgggaggcggcacaaactatccgcgaccatttcagggcaaagtcaccatgaccagggacacgtccaccgagacagcctatttagatgtcagaggacttacatatgacgacacggccgtctattattgtgtgagagacagggccaacggttcgggaagaagacgttttgagtcggtgaattggttcctggatctgtggggccgcggcacccaaataacagtcgtctcggcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 155 - protein, 205 aa, synthetic construct
  REGION 1..205: note = antibody variant
QSALTQPRSVSASPGQSVTISCTGTHNYVSWCQQKPGQAPKLLIYDFNKRPSGVSDRFSGSTSGNTASLTISGLQADDEGHYFCWAFENIGGGTKLTVLGQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 156 - DNA, 615 bp, synthetic construct
  misc_feature 1..615: note = antibody variant
  source 1..615: mol_type = other DNA
cagtctgccctaactcagcctcgctcagtgtccgcatctcctggtcagtcagtcaccatctcctgcactggaacacacaattatgtgtcctggtgtcaacagaaaccgggccaagcccccaaattattaatttacgatttcaataaacggccctcaggggtctctgatcgcttctctggctccacgtctggcaacacggcctccctgaccatctctggactccaggctgacgatgagggtcattatttttgttgggcgtttgaaaatatcggcggagggaccaagctgaccgtcctgggtcagcccaaggctgccccctcggtcactctgttcccaccctcctctgaggagcttcaagccaacaaggccacactggtgtgtctcgtaagtgacttctacccgggagccgtgacagtggcctggaaggcagatggcagccccgtcaaggtgggagtggagaccaccaaaccctccaaacaaagcaacaacaagtatgcggccagcagctacctgagcctgacgcccgagcagtggaagtcccacagaagctacagctgccgggtcacgcatgaagggagcaccgtggagaagacagtggcccctgcagaatgctct

SEQ ID NO: 157 - protein, 458 aa, synthetic construct; identical to SEQ ID NO: 153
  REGION 1..458: note = antibody variant

SEQ ID NO: 158 - DNA, 1374 bp, synthetic construct; identical to SEQ ID NO: 154
  misc_feature 1..1374: note = antibody variant
  source 1..1374: mol_type = other DNA

SEQ ID NO: 159 - protein, 205 aa, synthetic construct
  REGION 1..205: note = antibody variant
QSALTQPRSVSASPGQSVTISCTGTHNYVSWCQQKPGQAPKLLIYDFNKRPSGVSDRFSGSTSGNTASLTISGLQADDEGHYFCWAFENIGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 160 - DNA, 615 bp, synthetic construct
  misc_feature 1..615: note = antibody variant
  source 1..615: mol_type = other DNA
cagtctgccctaactcagcctcgctcagtgtccgcatctcctggtcagtcagtcaccatctcctgcactggaacacacaattatgtgtcctggtgtcaacagaaaccgggccaagcccccaaattattaatttacgatttcaataaacggccctcaggggtctctgatcgcttctctggctccacgtctggcaacacggcctccctgaccatctctggactccaggctgacgatgagggtcattatttttgttgggcgtttgaaaatatcggcggagggaccaagctgaccgtcctgggtcagcccaaggctgccccctcggtcactctgttcccgccctcctctgaggagcttcaagccaacaaggccacactggtgtgtctcataagtgacttctacccgggagccgtgacagtggcttggaaagcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 161 - protein, 457 aa, synthetic construct
  REGION 1..457: note = antibody variant
ADLVQSGAVVKKPGDSVRISCEAQGYRFPDYIIHWIRRAPGQGPEWMGWMNPMGGQVNIPWKFQGRVSMTRDTSIETAFLDLRGLKSDDTAVYYCVRDRSNGSGKRFESSNWFLDLWGRGTAVTIQSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 162 - DNA, 1371 bp, synthetic construct
  misc_feature 1..1371: note = antibody variant
  source 1..1371: mol_type = other DNA
gcggacttggtgcagtctggggctgtggtgaagaagcctggggactcagtgaggatctcctgtgaggctcaaggatatagatttcctgactacatcattcactggattcgacgggcccctggacaaggccctgaatggatgggatggatgaatccaatgggcggacaagtaaatattccatggaaatttcagggtagggtctccatgacccgggacacgtccatcgaaacagcatttctggacttaagaggactaaagtctgacgacacggccgtctattattgcgtgagagatcgcagtaatggatcgggaaagcgattcgagtcctccaattggttcctcgatctgtggggccgtgggactgcggtcacaattcaatcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 163 - protein, 203 aa, synthetic construct
  REGION 1..203: note = antibody variant
ALTQPRSVSASPGQSVTISCTGTHNLVSWCQHQPGRAPKLLIYDFNKRPSGVPDRFSGSGSGGTASLTITGLQDDDDAEYFCWAYEAFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 164 - DNA, 609 bp, synthetic construct
  misc_feature 1..609: note = antibody variant
  source 1..609: mol_type = other DNA
gccctgactcagcctcgctcagtgtccgcatctcctgggcagtccgtcaccatttcctgcactggaacccacaatttggtctcttggtgtcaacatcagccaggcagagcccccaaattattaatttatgacttcaataagaggccctcaggggtccctgatcgcttctctggctccgggtctggcggcacggcctccctgaccatcactggactccaggatgacgatgacgccgaatatttttgttgggcgtatgaagcttttggcggagggaccaagttgaccgttcttggtcagcccaaggctgccccctcggtcactctgttcccgccctcctctgaggagcttcaagccaacaaggccacactggtgtgtctcataagtgacttctacccgggagccgtgacagtggcttggaaagcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 165 - protein, 457 aa, synthetic construct
  REGION 1..457: note = antibody variant
ADLVQSGAVVKKPGDSVRISCEAQGYRFPDYIIHWIRRAPGQGPEWMGWMNPMGGQVNIPWKFQGRVSMTRDTSIETAFLDLRGLKSDDTAVYYCVRDRSNGSGKRFESSNWFLDLWGRGTAVTIQSSSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 166 - DNA, 1371 bp, synthetic construct
  misc_feature 1..1371: note = antibody variant
  source 1..1371: mol_type = other DNA
gcggacttggtgcagtctggggctgtggtgaagaagcctggggactcagtgaggatctcctgtgaggctcaaggatatagatttcctgactacatcattcactggattcgacgggcccctggacaaggccctgaatggatgggatggatgaatccaatgggcggacaagtaaatattccatggaaatttcagggtagggtctccatgacccgggacacgtccatcgaaacagcatttctggacttaagaggactaaagtctgacgacacggccgtctattattgcgtgagagatcgcagtaatggatcgggaaagcgattcgagtcctccaattggttcctcgatctgtggggccgtgggactgcggtcacaattcaatcatcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 167 - protein, 203 aa, synthetic construct; identical to SEQ ID NO: 4
  REGION 1..203: note = antibody variant

SEQ ID NO: 168 - DNA, 610 bp, synthetic construct
  misc_feature 1..610: note = antibody variant
  source 1..610: mol_type = other DNA
ggccctgactcagcctcgctcagtgtccgcatctcctgggcagtccgtcaccatttcctgcactggaacccacaatttggtctcttggtgtcaacatcagccaggcagagcccccaaattattaatttatgacttcaataagaggccctcaggggtccctgatcgcttctctggctccgggtctggcggcacggcctccctgaccatcactggactccaggatgacgatgacgccgaatatttttgttgggcgtatgaagcttttggcggagggaccaagttgaccgttcttcgtcagcccaaggctgccccctcggtcactctgttcccgccctcctctgaggagcttcaagccaacaaggccacactggtgtgtctcataagtgacttctacccgggagccgtgacagtggcttggaaagcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 169 - protein, 457 aa, synthetic construct
  REGION 1..457: note = antibody variant
ADLQSGAVVKKPGDSVRISCEAQGYRFPDYIIHWIRRAVPGQGPEWMGWMNPMGGQVNIPWKFQGRVSMTRDTSIETAFLDLRGLKSDDTAVYYCVRDRSNGSGKRFESSNWFLDLWGRGTAVTIQSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 170 - DNA, 1371 bp, synthetic construct; identical to SEQ ID NO: 162
  misc_feature 1..1371: note = antibody variant
  source 1..1371: mol_type = other DNA

SEQ ID NO: 171 - protein, 203 aa, synthetic construct
  REGION 1..203: note = antibody variant
ALTQPRSVSASPGQSVTISCTGTHNLVSWAQHQPGRAPKLLIYDFNKRPSGVPDRFSGSGSGGTASLTITGLQDDDDAEYFCWAYEAFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 172 - DNA, 609 bp, synthetic construct
  misc_feature 1..609: note = antibody variant
  source 1..609: mol_type = other DNA
gccctgactcagcctcgctcagtgtccgcatctcctgggcagtccgtcaccatttcctgcactggaacccacaatttggtctcttgggctcaacatcagccaggcagagcccccaaattattaatttatgacttcaataagaggccctcaggggtccctgatcgcttctctggctccgggtctggcggcacggcctccctgaccatcactggactccaggatgacgatgacgccgaatatttttgttgggcgtatgaagcttttggcggagggaccaagttgaccgttcttggtcagcccaaggctgccccctcggtcactctgttcccgccctcctctgaggagcttcaagccaacaaggccacactggtgtgtctcataagtgacttctacccgggagccgtgacagtggcttggaaagcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 173 - protein, 203 aa, synthetic construct
  REGION 1..203: note = antibody variant
ALTQPRSVSASPGQSVTISCTGTHNLVSWFQHQPGRAPKLLIYDFNKRPSGVPDRFSGSGSGGTASLTITGLQDDDDAEYFCWAYEAFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 174 - DNA, 1371 bp, synthetic construct; identical to SEQ ID NO: 162
  misc_feature 1..1371: note = antibody variant
  source 1..1371: mol_type = other DNA

SEQ ID NO: 175 - protein, 203 aa, synthetic construct; identical to SEQ ID NO: 173
  REGION 1..203: note = antibody variant

SEQ ID NO: 176 - DNA, 609 bp, synthetic construct
  misc_feature 1..609: note = antibody variant
  source 1..609: mol_type = other DNA
gccctgactcagcctcgctcagtgtccgcatctcctgggcagtccgtcaccatttcctgcactggaacccacaatttggtctcttggtttcaacatcagccaggcagagcccccaaattattaatttatgacttcaataagaggccctcaggggtccctgatcgcttctctggctccgggtctggcggcacggcctccctgaccatcactggactccaggatgacgatgacgccgaatatttttgttgggcgtatgaagcttttggcggagggaccaagttgaccgttcttggtcagcccaaggctgccccctcggtcactctgttcccgccctcctctgaggagcttcaagccaacaaggccacactggtgtgtctcataagtgacttctacccgggagccgtgacagtggcttggaaagcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 177 - protein, 457 aa, synthetic construct; identical to SEQ ID NO: 169
  REGION 1..457: note = antibody variant

SEQ ID NO: 178 - DNA, 1371 bp, synthetic construct; identical to SEQ ID NO: 162
  misc_feature 1..1371: note = antibody variant
  source 1..1371: mol_type = other DNA

SEQ ID NO: 179 - protein, 203 aa, synthetic construct
  REGION 1..203: note = antibody variant
ALTQPRSVSASPGQSVTISCTGTHNLVSWSQHQPGRAPKLLIYDFNKRPSGVPDRFSGSGSGGTASLTITGLQDDDDAEYFCWAYEAFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 180 - DNA, 609 bp, synthetic construct
  misc_feature 1..609: note = antibody variant
  source 1..609: mol_type = other DNA
gccctgactcagcctcgctcagtgtccgcatctcctgggcagtccgtcaccatttcctgcactggaacccacaatttggtctcttggtctcaacatcagccaggcagagcccccaaattattaatttatgacttcaataagaggccctcaggggtccctgatcgcttctctggctccgggtctggcggcacggcctccctgaccatcactggactccaggatgacgatgacgccgaatatttttgttgggcgtatgaagcttttggcggagggaccaagttgaccgttcttggtcagcccaaggctgccccctcggtcactctgttcccgccctcctctgaggagcttcaagccaacaaggccacactggtgtgtctcataagtgacttctacccgggagccgtgacagtggcttggaaagcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 181 - protein, 457 aa, synthetic construct; identical to SEQ ID NO: 169
  REGION 1..457: note = antibody variant

SEQ ID NO: 182 - DNA, 1371 bp, synthetic construct; identical to SEQ ID NO: 162
  misc_feature 1..1371: note = antibody variant
  source 1..1371: mol_type = other DNA

SEQ ID NO: 183 - protein, 203 aa, synthetic construct
  REGION 1..203: note = antibody variant
ALTQPRSVSASPGQSVTISCTGTHNLVSWYQHQPGRAPKLLIYDFNKRPSGVPDRFSGSGSGGTASLTITGLQDDDDAEYFCWAYEAFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 184 - DNA, 609 bp, synthetic construct
  misc_feature 1..609: note = antibody variant
  source 1..609: mol_type = other DNA
gccctgactcagcctcgctcagtgtccgcatctcctgggcagtccgtcaccatttcctgcactggaacccacaatttggtctcttggtatcaacatcagccaggcagagcccccaaattattaatttatgacttcaataagaggccctcaggggtccctgatcgcttctctggctccgggtctggcggcacggcctccctgaccatcactggactccaggatgacgatgacgccgaatatttttgttgggcgtatgaagcttttggcggagggaccaagttgaccgttcttggtcagcccaaggctgccccctcggtcactctgttcccgccctcctctgaggagcttcaagccaacaaggccacactggtgtgtctcataagtgacttctacccgggagccgtgacagtggcttggaaagcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 185 - protein, 457 aa, synthetic construct
  REGION 1..457: note = antibody variant
ADLVQSGAVVKKPGDSVRISCEAQGYRFPDYIIHWIRRAPGQGPEWMGWMNPTYGQVNIPWKFQGRVSMTRDTSIETAFLDLRGLKSDDTAVYYCVRDRSNGSGKRFESSNWFLDLWGRGTAVTIQSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 186 - DNA, 1371 bp, synthetic construct
  misc_feature 1..1371: note = antibody variant
  source 1..1371: mol_type = other DNA
gcggacttggtgcagtctggggctgtggtgaagaagcctggggactcagtgaggatctcctgtgaggctcaaggatatagatttcctgactacatcattcactggattcgacgggcccctggacaaggccctgaatggatgggatggatgaatccaacgtacggacaagtaaatattccatggaaatttcagggtagggtctccatgacccgggacacgtccatcgaaacagcatttctggacttaagaggactaaagtctgacgacacggccgtctattattgcgtgagagatcgcagtaatggatcgggaaagcgattcgagtcctccaattggttcctcgatctgtggggccgtgggactgcggtcacaattcaatcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 187 - protein, 203 aa, synthetic construct; identical to SEQ ID NO: 163
  REGION 1..203: note = antibody variant

SEQ ID NO: 188 - DNA, 609 bp, synthetic construct; identical to SEQ ID NO: 164
  misc_feature 1..609: note = antibody variant
  source 1..609: mol_type = other DNA

SEQ ID NO: 189 - protein, 457 aa, synthetic construct
  REGION 1..457: note = antibody variant
ADLVQSGAVVKKPGDSVRISCEAQGYRFPDYIIHWIRRAPGQGPEWMGWMNPMGGQVNIPWKFQGRVSMTRDTSIETAFLDLRGLKSDDTAVYYCVRDRSNGSGKRFESSNWFLDLWGRGTAVTIQSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 190 - DNA, 1371 bp, synthetic construct
  misc_feature 1..1371: note = antibody variant
  source 1..1371: mol_type = other DNA
gcggacttggtgcagtctggggctgtggtgaagaagcctggggactcagtgaggatctcctgtgaggctcaaggatatagatttcctgactacatcattcactggattcgacgggcccctggacaaggccctgaatggatgggatggatgaatccaatgggcggacaagtaaatattccatggaaatttcagggtagggtctccatgacccgggacacgtccatcgaaacagcatttctggacttaagaggactaaagtctgacgacacggccgtctattattgcgtgagagatcgcagtaatggatcgggaaagcgattcgagtcctccaattggttcctcgatctgtggggccgtgggactgcggtcacaattcaatcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgctgcatgaggctctgcacagccactacacgcagaagagcctctccctgtccccgggtaaa

SEQ ID NO: 191 - protein, 203 aa, synthetic construct; identical to SEQ ID NO: 163
  REGION 1..203: note = antibody variant

SEQ ID NO: 192 - DNA, 609 bp, synthetic construct; identical to SEQ ID NO: 164
  misc_feature 1..609: note = antibody variant
  source 1..609: mol_type = other DNA

SEQ ID NO: 193 - protein, 457 aa, synthetic construct
  REGION 1..457: note = antibody variant
ADLVQSGAVVKKPGDSVRISCEAQGYRFPDYIIHWIRRAPGQGPEWMGWMNPMGGQVNIPWKFQGRVSMTRDTSIETAFLDLRGLKSDDTAVYYCVRDRSNGSGKRFESSNWFLDLWGRGTAVTIQSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 194 - DNA, 1371 bp, synthetic construct
  misc_feature 1..1371: note = antibody variant
  source 1..1371: mol_type = other DNA
gcggacttggtgcagtctggggctgtggtgaagaagcctggggactcagtgaggatctcctgtgaggctcaaggatatagatttcctgactacatcattcactggattcgacgggcccctggacaaggccctgaatggatgggatggatgaatccaatgggcggacaagtaaatattccatggaaatttcagggtagggtctccatgacccgggacacgtccatcgaaacagcatttctggacttaagaggactaaagtctgacgacacggccgtctattattgcgtgagagatcgcagtaatggatcgggaaagcgattcgagtcctccaattggttcctcgatctgtggggccgtgggactgcggtcacaattcaatcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgctgcatgaggctctgcacagccactacacgcagaagagcctctccctgtccccgggtaaa

SEQ ID NO: 195 - protein, 203 aa, synthetic construct; identical to SEQ ID NO: 163
  REGION 1..203: note = antibody variant

SEQ ID NO: 196 - DNA, 609 bp, synthetic construct; identical to SEQ ID NO: 164
  misc_feature 1..609: note = antibody variant
  source 1..609: mol_type = other DNA

SEQ ID NO: 197 - protein, 457 aa, synthetic construct
  REGION 1..457: note = antibody variant
ADLVQSGAVVKKPGDSVRISCEAQGYRFPDYIIHWIRRAPGQGPEWMGWMNPMGGQVNIPWKFQGRVSMTRDTSIETAFLDLRGLKSDDTAVYYCVRDRSNGSGKRFESSNWFLDLWGRGTAVTIQSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 198 - DNA, 1371 bp, synthetic construct
  misc_feature 1..1371: note = antibody variant
  source 1..1371: mol_type = other DNA
gcggacttggtgcagtctggggctgtggtgaagaagcctggggactcagtgaggatctcctgtgaggctcaaggatatagatttcctgactacatcattcactggattcgacgggcccctggacaaggccctgaatggatgggatggatgaatccaatgggcggacaagtaaatattccatggaaatttcagggtagggtctccatgacccgggacacgtccatcgaaacagcatttctggacttaagaggactaaagtctgacgacacggccgtctattattgcgtgagagatcgcagtaatggatcgggaaagcgattcgagtcctccaattggttcctcgatctgtggggccgtgggactgcggtcacaattcaatcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctctatatcacccgggagcctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 199 - protein, 203 aa, synthetic construct; identical to SEQ ID NO: 163
  REGION 1..203: note = antibody variant

SEQ ID NO: 200 - DNA, 609 bp, synthetic construct; identical to SEQ ID NO: 164
  misc_feature 1..609: note = antibody variant
  source 1..609: mol_type = other DNA

SEQ ID NO: 201 - protein, 457 aa, synthetic construct; identical to SEQ ID NO: 161
  REGION 1..457: note = antibody variant

SEQ ID NO: 202 - DNA, 1371 bp, synthetic construct; identical to SEQ ID NO: 162
  misc_feature 1..1371: note = antibody variant
  source 1..1371: mol_type = other DNA

SEQ ID NO: 203 - protein, 205 aa, synthetic construct; identical to SEQ ID NO: 155
  REGION 1..205: note = antibody variant

SEQ ID NO: 204 - DNA, 615 bp, synthetic construct; identical to SEQ ID NO: 156
  misc_feature 1..615: note = antibody variant
  source 1..615: mol_type = other DNA

SEQ ID NO: 205 - protein, 457 aa, synthetic construct; identical to SEQ ID NO: 161
  REGION 1..457: note = antibody variant

SEQ ID NO: 206 - DNA, 1371 bp, synthetic construct; identical to SEQ ID NO: 162
  misc_feature 1..1371: note = antibody variant
  source 1..1371: mol_type = other DNA

SEQ ID NO: 207 - protein, 205 aa, synthetic construct; identical to SEQ ID NO: 8
  REGION 1..205: note = antibody variant

SEQ ID NO: 208 - DNA, 615 bp, synthetic construct; identical to SEQ ID NO: 84
  misc_feature 1..615: note = antibody variant
  source 1..615: mol_type = other DNA

SEQ ID NO: 209 - protein, 457 aa, synthetic construct; identical to SEQ ID NO: 165
  REGION 1..457: note = antibody variant

SEQ ID NO: 210 - DNA, 1371 bp, synthetic construct; identical to SEQ ID NO: 166
  misc_feature 1..1371: note = antibody variant
  source 1..1371: mol_type = other DNA

SEQ ID NO: 211 - protein, 206 aa, synthetic construct; identical to SEQ ID NO: 36
  REGION 1..206: note = antibody variant

SEQ ID NO: 212 - DNA, 618 bp, synthetic construct; identical to SEQ ID NO: 112
  misc_feature 1..618: note = antibody variant
  source 1..618: mol_type = other DNA

SEQ ID NO: 213 - protein, 457 aa, synthetic construct; identical to SEQ ID NO: 165
  REGION 1..457: note = antibody variant

SEQ ID NO: 214 - DNA, 1371 bp, synthetic construct; identical to SEQ ID NO: 166
  misc_feature 1..1371: note = antibody variant
  source 1..1371: mol_type = other DNA

SEQ ID NO: 215 - protein, 205 aa, synthetic construct; identical to SEQ ID NO: 14
  REGION 1..205: note = antibody variant

SEQ ID NO: 216 - DNA, 615 bp, synthetic construct; identical to SEQ ID NO: 90
  misc_feature 1..615: note = antibody variant
  source 1..615: mol_type = other DNA

SEQ ID NO: 217 - protein, 460 aa, synthetic construct
  REGION 1..460: note = antibody variant
ADLVQSGAVVKKPGDSVRISCEAQGYRFPDYIIHWIRRAPGQGPEWMGWMNPMGGQVNIPWKFQGRVSMTRDTSIETAFLDLRGLKSDDTAVYYCVRDRSNGSGKRFESSNWFLDLWGRGTAVTIQSASTKGPSVFPLAPSSRSTSESTAALGCLVKDYFPEPVTVSWNSGSLTSGVHTFPAVLQSSGLYSLSSVVTVPSSSLGTQTYVCNVNHKPSNTKVDKRVEIKTCGGGSKPPTCPPCPAPELLGGPSVFLFPPKPKDTLMISRTPEVTCVVVDVSQEDPDVKFNWYVNGAEVHHAQTKPRETQYNSTYRVVSVLTVTHQDWLNGKEYTCKVSNKALPAPIQKTISKDKGQPREPQVYTLPPSREELTKNQVSLTCLVKGFYPSDIVVEWESSGQPENTYKTTPPVLDSDGSYFLYSKLTVDKSRWQQGNVFSCSVLHEALHSHYTQKSLSLSPGK

SEQ ID NO: 218 - DNA, 1380 bp, synthetic construct
  misc_feature 1..1380: note = antibody variant
  source 1..1380: mol_type = other DNA
gcggacttggtgcagtctggggctgtggtgaagaagcctggggactcagtgaggatctcctgtgaggctcaaggatatagatttcctgactacatcattcactggattcgacgggcccctggacaaggccctgaatggatgggatggatgaatccaatgggcggacaagtaaatattccatggaaatttcagggtagggtctccatgacccgggacacgtccatcgaaacagcatttctggacttaagaggactaaagtctgacgacacggccgtctattattgcgtgagagatcgcagtaatggatcgggaaagcgattcgagtcctccaattggttcctcgatctgtggggccgtgggactgcggtcacaattcaatcagcctcgaccaagggcccatcggtcttccccctggcgccctcctccaggagcacctccgagagcacagcggccctgggctgcctggtcaaggactacttccctgaacccgtgaccgtgtcgtggaactcaggctccctgaccagcggcgtgcacaccttcccggctgtcctacagtcctcagggctctactccctcagcagcgtggtgaccgtgccctccagcagcttgggcacccagacctacgtctgcaacgtaaaccacaagcccagcaacaccaaggtggacaagagagttgagataaaaacatgtggtggtggcagcaaacctcccacgtgcccaccgtgcccagcacctgaactcctggggggaccgtcagtcttcctcttccccccaaaacccaaggacaccctcatgatctcccggacccctgaggtcacatgcgtggtggtagacgtgagccaggaagaccccgatgtcaagttcaactggtacgtaaatggcgcggaggtgcatcatgcccagacgaagccacgggagacgcagtacaacagcacatatcgtgtggtcagcgtcctcaccgtcacgcaccaggactggctgaacggcaaggagtacacgtgcaaggtctccaacaaagccctcccggcccccatccagaaaaccatctccaaagacaaagggcagccccgagagcctcaggtgtacaccctgcccccgtcccgggaggagctgaccaagaaccaggtcagcctgacctgcctggtcaaaggcttctaccccagtgacatcgtcgtggagtgggagagcagcgggcagccggagaacacctacaagaccaccccgcccgtgctggactccgacggctcctacttcctctacagcaagctcaccgtggacaagagcaggtggcagcaggggaacgtcttctcatgctccgtgctgcatgaggctctgcacagccactacacgcagaagagcctctccctgtccccgggtaaa

SEQ ID NO: 219 - protein, 203 aa, synthetic construct
  REGION 1..203: note = antibody variant
ALTQPRSVSASPGQSVTISCTGTHNLVSWCQHQPGRAPKLLIYDFNKRPSGVPDRFSGSGSGGTASLTITGLQDDDDAEYFCWAYEAFGGGTKLTVLGQPKAAPSVTLFPPSSEELQANKATLVCLISDFYPGAVEVAWKADGSAVNAGVETTKPSKQSNNKYAASSYLSLTSDQWKSHKSYSCQVTHEGSTVEKTVAPAECS

SEQ ID NO: 220 - DNA, 609 bp, synthetic construct
  misc_feature 1..609: note = antibody variant
  source 1..609: mol_type = other DNA
gccctgactcagcctcgctcagtgtccgcatctcctgggcagtccgtcaccatttcctgcactggaacccacaatttggtctcttggtgtcaacatcagccaggcagagcccccaaattattaatttatgacttcaataagaggccctcaggggtccctgatcgcttctctggctccgggtctggcggcacggcctccctgaccatcactggactccaggatgacgatgacgccgaatatttttgttgggcgtatgaagcttttggcggagggaccaagttgaccgttcttggtcagcccaaggctgccccctcggtcactctgttcccgccctcgagcgaggagcttcaagccaacaaggccacactagtgtgtctgatcagtgacttctacccgggagccgtggaagtggcctggaaggcagatggcagcgctgtcaacgcgggagtggagaccaccaaaccctccaaacagagcaacaacaagtacgcggccagcagctacctgagcctgacgtccgaccagtggaagtcccacaagagctacagctgccaggtcacgcacgaagggagcaccgtggagaagacagtggcccctgcagaatgttca

SEQ ID NO: 221 - protein, 457 aa, synthetic construct
  REGION 1..457: note = antibody variant
ADLVQSGAVVKKPGDSVRISCEAQGYKFPDYIIHWIRRAPGQGLEWMGWINPMGGQVNIPWQFQGRVSMTRDTSIETAFLDLRGLKSDDTALYYCVRDRSNGSGRRFESSNWFLDLWGRGTAVTVHSPSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 222 - DNA, 1371 bp, synthetic construct
  misc_feature 1..1371: note = antibody variant
  source 1..1371: mol_type = other DNA
gcggacttggtgcagtctggggctgtggtgaagaagcctggggactcagtgaggatctcctgtgaggctcaaggatacaaatttcctgactacatcattcactggattcgacgcgcccctggacaaggccttgagtggatggggtggattaatccaatgggcggacaagtaaacattccatggcagtttcagggcagggtctccatgacccgggacacgtccatcgaaacagcatttctggacttaagaggactaaagtctgacgacacggccctctattattgcgtgagagatcgaagtaatggatcgggaaggcgattcgagtcctccaattggttcctcgatctgtggggccgcggcactgcggtcactgttcattcaccctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 223 - protein, 205 aa, synthetic construct; identical to SEQ ID NO: 6
  REGION 1..205: note = antibody variant

SEQ ID NO: 224 - DNA, 615 bp, synthetic construct; identical to SEQ ID NO: 82
  misc_feature 1..615: note = antibody variant
  source 1..615: mol_type = other DNA

SEQ ID NO: 225 - protein, 458 aa, synthetic construct
  REGION 1..458: note = antibody variant
SAELVQSGAVVKKPGTSVKVSCQAYGYTFTDYLIHWLRQAPGQGLEWMGWMNPVYGQVNYAQNFQGRVSMTRDIYRETAFLEVRDLKTDDTGTYYCVRDTGDGSRRHFDSINWFLDLWGRGTWIRVAPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 226 - DNA, 1374 bp, synthetic construct
  misc_feature 1..1374: note = antibody variant
  source 1..1374: mol_type = other DNA
tcggcggaattggtgcaatctggggctgtggtgaagaagcctgggacctccgtgaaggtctcttgtcaggcttatggatacacttttaccgactaccttattcattggcttcgacaggcccctggacaaggacttgaatggatgggatggatgaatcctgtttatggacaagtaaattatgcccaaaactttcagggcagggtctccatgaccagggacatttacagggaaacagcatttctagaggtgcgcgacctgaagactgacgacacaggcacttattattgtgtgagagacacaggcgacggttcgcggagacactttgactccatcaattggtttctcgatctttggggccgcgggacatggataagggtcgccccagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 227 - protein, 205 aa, synthetic construct; identical to SEQ ID NO: 8
  REGION 1..205: note = antibody variant

SEQ ID NO: 228 - DNA, 615 bp, synthetic construct; identical to SEQ ID NO: 84
  misc_feature 1..615: note = antibody variant
  source 1..615: mol_type = other DNA

SEQ ID NO: 229 - protein, 457 aa, synthetic construct
  REGION 1..457: note = antibody variant
ADLVQSGAVMKKPGDSVRISCEARGYTFTDYVIHWIRRAPGQGLEWMGWIDPPYGQVNIPWNFQGRVSMTRDTSIETAFLDLRGLKSDDTGLYYCVRDRSNGWGKRFESSNWFLDLWGRGTVVTVHSPSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 230 - DNA, 1371 bp, synthetic construct
  misc_feature 1..1371: note = antibody variant
  source 1..1371: mol_type = other DNA
gcggacttggtgcagtctggggctgtgatgaagaagcctggggactcagtgagaatctcctgtgaggctcgaggatacacattcactgactacgtcattcactggattcgacgcgcccctggacaaggccttgaatggatggggtggattgatccaccttatggacaagtaaatattccatggaattttcagggcagggtctccatgacccgggacacgtccatcgaaacagcatttctggacttaagaggtctaaagtctgacgacacgggcctctattattgcgtgagagatcgaagtaatggatggggaaagcgattcgagtcctccaattggttcctcgatctgtggggccgcggcactgtggtcactgttcactcaccctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 231 - protein, 205 aa, synthetic construct; identical to SEQ ID NO: 6
  REGION 1..205: note = antibody variant

SEQ ID NO: 232 - DNA, 615 bp, synthetic construct; identical to SEQ ID NO: 86
  misc_feature 1..615: note = antibody variant
  source 1..615: mol_type = other DNA

SEQ ID NO: 233 - protein, 457 aa, synthetic construct; identical to SEQ ID NO: 229
  REGION 1..457: note = antibody variant

SEQ ID NO: 234 - DNA, 1371 bp, synthetic construct; identical to SEQ ID NO: 230
  misc_feature 1..1371: note = antibody variant
  source 1..1371: mol_type = other DNA

SEQ ID NO: 235 - protein, 205 aa, synthetic construct
  REGION 1..205: note = antibody variant
QSALTQPRSVSASPGQSVTISCTGTHNLVSWCQHHPGRAPKLLIYDFNKRPSGVPDRFSGSGSGGTASLTISGLQDDDDAEYFCWAYEAFGGGTKLTVLRQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 236 - DNA, 615 bp, synthetic construct
  misc_feature 1..615: note = antibody variant
  source 1..615: mol_type = other DNA
cagtctgccctgactcagcctcgctcagtgtccgcatctcctgggcagtccgtcaccatttcctgcactggaacccacaatttggtctcttggtgtcaacatcacccaggcagagcccccaagttattaatttatgacttcaataagaggccctcaggggtccctgatcgcttctctggctccgggtctggcggcacggcctccctaaccatcagtggactccaggatgacgatgacgccgaatatttttgttgggcatatgaagctttcggcggagggaccaagttgactgtacttcgtcagcccaaggctgccccctcggtcactctgttcccaccctcctctgaggagcttcaagccaacaaggccacactggtgtgtctcgtaagtgacttctacccgggagccgtgacagtggcctggaaggcagatggcagccccgtcaaggtgggagtggagaccaccaaaccctccaaacaaagcaacaacaagtatgcggccagcagctacctgagcctgacgcccgagcagtggaagtcccacagaagctacagctgccgggtcacgcatgaagggagcaccgtggagaagacagtggcccctgcagaatgctct

SEQ ID NO: 237 - protein, 457 aa, synthetic construct
  REGION 1..457: note = antibody variant
ADLVQSGAVVKKPGDSVRISCEAQGYTFTDYVIHWIRRAPGQGLEWMGWINPGYGQVNIPWNFQGRVSMTRDTSIETAFLDLRGLKSDDTGLYYCVRDRSNGWGKRFESSNWFLDLWGRGTVVTVHSPSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 238 - DNA, 1371 bp, synthetic construct
  misc_feature 1..1371: note = antibody variant
  source 1..1371: mol_type = other DNA
gcggacttggtgcagtctggggctgtggtgaagaagcctggggactcagtgagaatctcctgtgaggctcaaggatacacattcactgactacgtcattcactggattcgacgcgcccctggacaaggccttgaatggatggggtggattaatccaggttatggacaagtaaatattccatggaactttcagggcagggtctccatgacccgagacacgtccatcgaaacagcatttctggacttaagaggtctaaagtctgacgacacgggcctctattattgcgtgagagatcgaagtaatggatggggaaagcgattcgagtcctccaattggttcctcgatctgtggggccgcggcactgtggtcactgttcactcaccctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 239 - protein, 205 aa, synthetic construct; identical to SEQ ID NO: 12
  REGION 1..205: note = antibody variant

SEQ ID NO: 240 - DNA, 615 bp, synthetic construct; identical to SEQ ID NO: 88
  misc_feature 1..615: note = antibody variant
  source 1..615: mol_type = other DNA

SEQ ID NO: 241 - protein, 457 aa, synthetic construct
  REGION 1..457: note = antibody variant
ADLVQSGAVVKNAGASVRVSCEAYGYTFVDYFIHWVRQAPGQGFEWMGYMDPLNGRPNIARKFQGRLSLSRDRSSETSFLDLSGLRSDDSAVYYCVRDKSNGSGRRFDSSNWFLDLWGRGTRVSIFSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 242 - DNA, 1371 bp, synthetic construct
  misc_feature 1..1371: note = antibody variant
  source 1..1371: mol_type = other DNA
gcggacttggtgcagtctggggctgtggtgaaaaatgctggggcctcagtgagggtctcctgtgaggcttatggatacacattcgtggactacttcattcattgggtccgacaggcccctggacaaggctttgaatggatgggatacatggatcccttgaacgggcgcccaaacattgcgcgaaaatttcagggcaggctctccctgagtcgagataggtccagcgaaacttcatttctggacttaagtggactgaggtctgacgactcggccgtctattattgtgtgagagacaagagtaatggatcgggcagacggtttgactcgtctaattggtttctcgatctgtggggccgtggaacccgggtcagtattttctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 243 - protein, 205 aa, synthetic construct; identical to SEQ ID NO: 14
  REGION 1..205: note = antibody variant

SEQ ID NO: 244 - DNA, 615 bp, synthetic construct; identical to SEQ ID NO: 90
  misc_feature 1..615: note = antibody variant
  source 1..615: mol_type = other DNA

SEQ ID NO: 245 - protein, 457 aa, synthetic construct
  REGION 1..457: note = antibody variant
ADLVQSGAVVKKPGDSVRVSCEAYGYTFSDYIIHWIRRAPGRGLEWMGWMNPMGGQVNIPWNFQGRVSMTRDTSIETAFLDLRGLRSDDTAVYYCVRDRSNGSGKRFESSNWFLDLWGRGTAVTISSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 246 - DNA, 1371 bp, synthetic construct
  misc_feature 1..1371: note = antibody variant
  source 1..1371: mol_type = other DNA
gcggacttggtgcagtctggggctgtggtgaagaagcctggggactcagtgagggtctcctgtgaggcttatggatacacattcagtgactacatcattcattggattcgacgggcccctggacgaggccttgaatggatgggatggatgaatccgatgggcggacaagtgaatattccgtggaactttcaggggagagtctccatgacccgggacacgtccatcgaaacagcatttctggacttaagaggactgaggtctgacgacacggccgtctattactgtgtgagagatcgcagcaatggatcgggcaagcgatttgagtcctccaattggttcctcgatctgtggggccgcgggaccgcggtcactatttcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 247 - protein, 205 aa, synthetic construct
  REGION 1..205: note = antibody variant
QSALTQPRSVSAAPGQSVTISCTGTHNLVSWCQHHPGRAPKLLIYDFNKRPSGVPDRFSGSGSGGTASLTITGLQDDDEAEYFCWAYEVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 248 - DNA, 615 bp, synthetic construct
  misc_feature 1..615: note = antibody variant
  source 1..615: mol_type = other DNA
cagtctgccctgactcagcctcgctcagtgtccgcagctcctggacagtccgtcaccatttcctgcactggaacccacaatttggtttcttggtgtcaacatcacccaggcagagcccccaagttattaatttatgacttcaataagagaccctcaggtgtccctgatcgtttctctggctccgggtctggcggcacggcctccctaaccatcactggactccaggatgacgatgaggctgaatatttttgttgggcgtatgaagtttttggcggagggaccaagttgaccgtgcttggtcagcccaaggctgccccctcggtcactctgttcccgccctcctctgaggagcttcaagccaacaaggccacactggtgtgtctcataagtgacttctacccgggagccgtgacagtggcttggaaagcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 249 - protein, 457 aa, synthetic construct
  REGION 1..457: note = antibody variant
ADLVQSGAVVKTPGASVRVSCEAYGYTFIDYIIHWVRQAPGQGFEWLGYIDPMNGRPNIARKFQGRLSLSRDTSIETSFLDLSGLRSDDSAVYYCVRDKSNGSGKRFDSSNWFLDLWGRGTRVSISSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 250 - DNA, 1371 bp, synthetic construct
  misc_feature 1..1371: note = antibody variant
  source 1..1371: mol_type = other DNA
gcggacttggtgcagtctggggctgtggtgaagacgcctggggcctcagtgagggtctcctgtgaggcttatggatacacattcattgactacatcattcattgggtccgacaggcccctggacaaggttttgaatggctgggatacattgatcctatgaacgggcgcccaaacattgcgcgaaaatttcagggcaggctctccctgagccgggatacgtccatcgaaacatcatttctggacttaagtggactgaggtctgacgactcggccgtctattattgtgtgagagacaagagtaatggatcgggcaaacgatttgactcctctaattggtttctcgatctgtggggccgtggaacgcgggtcagcatttcttcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 251 - protein, 205 aa, synthetic construct
  REGION 1..205: note = antibody variant
QSALTQPRSVSAAPGQSVTISCTGTHNYVSWCQQHPGRAPKLLIYDFNKRPSGVPDRFSGSGSGGTASLTITRLQDDDDADYFCWAYDAFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 252 - DNA, 615 bp, synthetic construct
  misc_feature 1..615: note = antibody variant
  source 1..615: mol_type = other DNA
cagtctgccctgactcagcctcgctcagtgtccgcagctcctggacagtcagtcaccatctcctgcactggaacccacaattatgtctcttggtgtcaacaacatccaggcagagcccccaaattactaatttatgacttcaataagaggccctcaggggtcccggatcgcttctctggctccggatctggcggcacggcctccctaaccatcactagactccaggatgacgatgacgctgactatttttgttgggcgtatgatgcttttggcggagggaccaagttgaccgtcctgggtcagcccaaggctgccccctcggtcactctgttcccgccctcctctgaggagcttcaagccaacaaggccacactggtgtgtctcataagtgacttctacccgggagccgtgacagtggcttggaaagcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 253 - protein, 457 aa, synthetic construct; identical to SEQ ID NO: 249
  REGION 1..457: note = antibody variant

SEQ ID NO: 254 - DNA, 1371 bp, synthetic construct; identical to SEQ ID NO: 250
  misc_feature 1..1371: note = antibody variant
  source 1..1371: mol_type = other DNA

SEQ ID NO: 255 - protein, 205 aa, synthetic construct; identical to SEQ ID NO: 18
  REGION 1..205: note = antibody variant

SEQ ID NO: 256 - DNA, 615 bp, synthetic construct; identical to SEQ ID NO: 94
  misc_feature 1..615: note = antibody variant
  source 1..615: mol_type = other DNA

SEQ ID NO: 257 - protein, 457 aa, synthetic construct
  REGION 1..457: note = antibody variant
ADLVQSGAVVKKPGASVRVSCEAYGYTFTDYIIHWIRQAPGQGLEWMGWMNPMGGRTNIPWKFQGRVSMTRDTSIETAFLDLSGLTSDDTAVYYCVRDKSNGSGKRFDSSNWFLDLWGRGTPVTISSPSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 258 - DNA, 1371 bp, synthetic construct
  misc_feature 1..1371: note = antibody variant
  source 1..1371: mol_type = other DNA
gcggacttggtgcagtctggggctgtggtgaagaagcctggggcctcagtgagggtctcctgtgaggcttatggatacacattcactgactacatcattcattggattcgacaggcccctggacaaggccttgaatggatgggatggatgaatcctatgggcgggcgcacaaatattccgtggaaatttcagggcagggtctccatgacccgggacacgtccatcgaaacagcatttctggacttaagtggactaacgtctgacgacacggccgtctattattgcgtgagagacaagagtaatggatcgggcaaacgatttgactcctctaattggttcctcgatctgtggggccgcggaaccccggtcactatttcctcaccctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 259 - protein, 205 aa, synthetic construct; identical to SEQ ID NO: 20
  REGION 1..205: note = antibody variant

SEQ ID NO: 260 - DNA, 615 bp, synthetic construct; identical to SEQ ID NO: 96
  misc_feature 1..615: note = antibody variant
  source 1..615: mol_type = other DNA

SEQ ID NO: 261 - protein, 457 aa, synthetic construct
  REGION 1..457: note = antibody variant
ADLVQSGAVVKKPGASVRVSCEAYGYTFVDYLIHWVRQAPGQGFEWMGYMDPMNGRPNIARKFQGRLSLSRDTSIETSFLDLSGLRSDDSAVYYCVRDKSGGSGKLFDSSNWFLDLWGRGTRVSISSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 262 - DNA, 1371 bp, synthetic construct
  misc_feature 1..1371: note = antibody variant
  source 1..1371: mol_type = other DNA
gcggacttggtgcagtctggggctgtggtgaagaagcctggggcctcagtgcgggtctcctgtgaggcttatggatatacattcgttgactacctcattcattgggtccgacaggcccccggacaaggttttgaatggatgggatacatggatcctatgaacgggcgcccaaatattgcgcgaaaatttcagggcaggctctccctgagccgagatacgtccatcgaaacatcatttctggacttaagtggactgaggtctgacgactcggccgtctattattgtgtgagagacaagagtggtggatcgggcaaactatttgactcctctaattggtttctcgatctgtggggccgtggaacccgggtcagcatttcttcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 263 - protein, 205 aa, synthetic construct; identical to SEQ ID NO: 22
  REGION 1..205: note = antibody variant

SEQ ID NO: 264 - DNA, 615 bp, synthetic construct; identical to SEQ ID NO: 98
  misc_feature 1..615: note = antibody variant
  source 1..615: mol_type = other DNA

SEQ ID NO: 265 - protein, 457 aa, synthetic construct
  REGION 1..457: note = antibody variant
ADLVQSGAVVKKPGDSVRISCEAQGYTFTDYVIHWIRRAPGQGLEWMGWINPGYGQVNIPWNFQGRVSMTRDTSIETAFLELRGLKSDDTGLYYCVRDRSNGWGKRFESSNWFLDLWGRGTVITVHSPSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 266 - DNA, 1371 bp, synthetic construct
  misc_feature 1..1371: note = antibody variant
  source 1..1371: mol_type = other DNA
gcggacttggtgcagtctggggctgtggtgaagaagcctggggactcagtgagaatctcctgtgaggctcaaggatacacattcactgactacgtcattcactggattcgacgggcccctggacaaggccttgaatggatggggtggattaatccaggttatggacaagtaaatattccatggaactttcagggcagggtctccatgacccgagacacgtccatcgaaacagcttttctggagttaagaggtctaaagtctgacgacacgggcctctattattgcgtgagagatcgaagtaatggatggggaaagcgattcgagtcctccaattggttcctcgatctgtggggccgcggcactgtgattactgttcactcaccctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 267 - protein, 205 aa, synthetic construct; identical to SEQ ID NO: 24
  REGION 1..205: note = antibody variant

SEQ ID NO: 268 - DNA, 615 bp, synthetic construct; identical to SEQ ID NO: 100
  misc_feature 1..615: note = antibody variant
  source 1..615: mol_type = other DNA

SEQ ID NO: 269 - protein, 457 aa, synthetic construct
  REGION 1..457: note = antibody variant
ADLVQSGAVVKKPGDSVRISCEAQGYTFTDYVIHWIRRAPGQGLEWMGWMDPSYGQVNIPRNFQGRVSMTRDTFRETAYLELRGLQSDDKGLYYCVRDRSHGSGRQFESSNWFLDLWGRGTVVNVQSPSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 270 - DNA, 1371 bp, synthetic construct
  misc_feature 1..1371: note = antibody variant
  source 1..1371: mol_type = other DNA
gcggacttggtgcagtctggggctgtggtgaagaagcctggggactcagtgagaatctcctgtgaggctcagggatatacattcaccgactacgtcattcattggattcgacgggcccctggacaaggccttgaatggatggggtggatggatccaagttatggacaagtcaatattccacggaactttcagggcagggtctccatgacccgggacacgttcagggaaacagcatatctggaattaagaggtctacagtctgacgacaagggcctctattattgtgtgagagatcgaagtcacggatcgggaaggcaattcgagtcctccaactggttcctcgatctgtggggccgcggcactgtggtcaatgttcagtcaccctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 271 - protein, 205 aa, synthetic construct; identical to SEQ ID NO: 26
  REGION 1..205: note = antibody variant

SEQ ID NO: 272 - DNA, 615 bp, synthetic construct; identical to SEQ ID NO: 102
  misc_feature 1..615: note = antibody variant
  source 1..615: mol_type = other DNA

SEQ ID NO: 273 - protein, 457 aa, synthetic construct
  REGION 1..457: note = antibody variant
ADLVQSGAVVKKPGDSLRISCEAQGYTFTDYVIHWIRRAPGQGLEWMGWMDPSFGQMNIPRNFQGRVSMTRDMYIETAFLDLRGLKSDDTGLYYCVRDRSHGSGRLFESSNWFLDLWGRGTVVTVQSPSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 274 - DNA, 1371 bp, synthetic construct
  misc_feature 1..1371: note = antibody variant
  source 1..1371: mol_type = other DNA
gcggacttggtgcagtctggggctgtggtgaagaagcctggggactcactgagaatctcctgtgaggctcaaggatacacattcactgactacgtcattcactggattcgacgggcccctggacaaggccttgaatggatgggatggatggatccaagttttggacaaatgaacattccacggaactttcagggcagggtctccatgacccgtgacatgtacatcgaaacagcatttctggacttaagaggtctaaagtctgacgacacgggcctctattattgcgtgagagatcgaagtcatggatcgggaaggctattcgagtcctccaattggttcctcgatctgtggggccgcggcactgtggtcactgttcagtcaccctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 275 - protein, 205 aa, synthetic construct; identical to SEQ ID NO: 26
  REGION 1..205: note = antibody variant

SEQ ID NO: 276 - DNA, 615 bp, synthetic construct; identical to SEQ ID NO: 102
  misc_feature 1..615: note = antibody variant
  source 1..615: mol_type = other DNA

SEQ ID NO: 277 - protein, 457 aa, synthetic construct
  REGION 1..457: note = antibody variant
ADLVQSGAVVKKPGDSVRISCEAQGYRFTDYVIHWIRRAPGQGLEWMGLMDPSFGRMNIPRKFQGRVSMTRDTSMETAFLDFRGLNFDDTGLYYCVRDRSHGSGRLFESSNWFLDLWGRGTVVTVQSPSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 278 - DNA, 1371 bp, synthetic construct
  misc_feature 1..1371: note = antibody variant
  source 1..1371: mol_type = other DNA
gcggacttggtgcagtctggggctgtggtgaagaagcctggggactcagtgagaatctcctgtgaggctcaaggatacagattcactgactacgtcattcattggattcgacgggcccctggacaaggccttgaatggatggggttgatggatccaagttttggacgaatgaatattccacggaaatttcagggcagggtctccatgacccgggacacgtccatggaaacagcatttctggacttcagaggtctaaattttgacgacacgggcctctattattgcgtgagagatcgaagtcatggatcgggaagactattcgagtcctccaattggttcctcgatctgtggggccgcggcactgtggtcactgttcagtcaccctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 279 - protein, 205 aa, synthetic construct; identical to SEQ ID NO: 26
  REGION 1..205: note = antibody variant

SEQ ID NO: 280 - DNA, 615 bp, synthetic construct; identical to SEQ ID NO: 106
  misc_feature 1..615: note = antibody variant
  source 1..615: mol_type = other DNA

SEQ ID NO: 281 - protein, 457 aa, synthetic construct
  REGION 1..457: note = antibody variant
ADLVQSGAVVKKPGDSVRISCEAQGYTFIDYVIHWIRRAPGQGLEWMGLMDPTYGRMNIPRKFQGRVSMTRDTSIETAFLDLRGLKSDDTGLYYCVRDRSHGSGRLFESSNWFLDLWGRGTVVTVQSPSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 282 - DNA, 1371 bp, synthetic construct
  misc_feature 1..1371: note = antibody variant
  source 1..1371: mol_type = other DNA
gcggacttggtgcagtctggggctgtggtgaagaagcctggggactcagtgagaatttcctgtgaggctcaaggatacacattcattgactacgtcattcactggattcgacgggcccctggacaaggccttgaatggatggggttgatggatccaacttatggacgaatgaatattccacggaagtttcagggcagggtctccatgacccgggacacgtccatcgaaacagcatttctggacttaagaggtctaaaatctgacgacacgggcctctattattgcgtgagagatcgaagtcatggatcgggaaggctattcgagtcctccaactggttcctggatctgtggggccgcggcactgtggtcactgttcagtcaccctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 283 - protein, 205 aa, synthetic construct; identical to SEQ ID NO: 26
  REGION 1..205: note = antibody variant

SEQ ID NO: 284 - DNA, 615 bp, synthetic construct; identical to SEQ ID NO: 108
  misc_feature 1..615: note = antibody variant
  source 1..615: mol_type = other DNA

SEQ ID NO: 285 - protein, 457 aa, synthetic construct
  REGION 1..457: note = antibody variant
ADLVQSGAVVKKPGDSVRISCEAQGYRFLDYIIHWIRRAPGQGLEWMGWMNPMGGQVNIPWNFQGRVSMTRDTSIETAFLDLRGLKSDDTAVYYCVRDRSNGSGKRFESSNWFLDLWGRGTAVTIHSPSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 286 - DNA, 1371 bp, synthetic construct
  misc_feature 1..1371: note = antibody variant
  source 1..1371: mol_type = other DNA
gcggacttggtgcagtctggggctgtggtgaagaagcctggggactcagtgaggatctcctgtgaggctcaaggatacagatttcttgactacatcattcactggattcgacgagcccctggacaaggccttgaatggatgggatggatgaatccaatgggcggacaagtaaacattccatggaactttcagggtagggtctccatgacccgggacacgtccatcgaaacagcatttctggacttaagaggactaaagtctgacgacacggccgtctattattgcgtgagagatcgcagtaatggatcgggaaagcgattcgagtcctccaattggttcctcgatctgtggggccgcgggactgcggtcactattcattcaccctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 287 - protein, 205 aa, synthetic construct; identical to SEQ ID NO: 34
  REGION 1..205: note = antibody variant

SEQ ID NO: 288 - DNA, 615 bp, synthetic construct; identical to SEQ ID NO: 110
  misc_feature 1..615: note = antibody variant
  source 1..615: mol_type = other DNA

SEQ ID NO: 289 - protein, 457 aa, synthetic construct
  REGION 1..457: note = antibody variant
ADLVQSGAVVKKPGDSVRISCEAQGYRFLDYIIHWIRRAPGQGLEWMGWMNPMGGQVNIPWNFQGRVSMTRDTSIETAFLDLRGLKSDDTAVYYCVRDRSNGSGKRFESSNWFLDLWGRGTAVTIHSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 290 - DNA, 1371 bp, synthetic construct
  misc_feature 1..1371: note = antibody variant
  source 1..1371: mol_type = other DNA
gcggacttggtgcagtctggggctgtggtgaagaagcctggggactcagtgaggatctcctgtgaggctcaaggatacagatttcttgactacatcattcactggattcgacgagcccctggacaaggccttgaatggatgggatggatgaatccaatgggcggacaagtaaacattccatggaactttcagggtagggtctccatgacccgggacacgtccatcgaaacagcatttctggacttaagaggactaaagtctgacgacacggccgtctattattgcgtgagagatcgcagtaatggatcgggaaagcgattcgagtcctccaattggttcctcgatctgtggggccgcgggactgcggtcactattcattcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 291 - protein, 205 aa, synthetic construct
  REGION 1..205: note = antibody variant
QSALTQPRSVSASPGQSVTISCTGTHNLVSWCQHHPGRAPKLLIYDFNKRPSGVPDRFSGSGSGGTASLTITGLQDDDDAEYFCWAYEAFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 292 - DNA, 615 bp, synthetic construct
  misc_feature 1..615: note = antibody variant
  source 1..615: mol_type = other DNA
cagtctgccctgactcagcctcgctcagtgtccgcatctcctgggcagtccgtcaccatttcctgcactggaacccacaatttggtctcttggtgtcaacatcacccaggcagagcccccaaattattaatttatgacttcaataagaggccctcaggggtccctgatcgcttctctggctccgggtctggcggcacggcctccctgaccatcactggactccaggatgacgatgacgccgaatatttttgttgggcgtatgaagcttttggcggagggaccaagttgaccgtacttggtcagcccaaggctgccccctcggtcactctgttcccgccctcctctgaggagcttcaagccaacaaggccacactggtgtgtctcataagtgacttctacccgggagccgtgacagtggcttggaaagcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 293 - protein, 450 aa, synthetic construct
  REGION 1..450: note = antibody variant
HVQLVQSGGGVKKIGAAVRISCEVTGYKFMDQLINWVRQAPGQGLEWMGWMNPTYGQVNYSWRFEGRVTMTRDMDTETAFMELRGLRVDDTAVYYCARGPSGENYPFHYWGQGVRVVVSSPSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 294 - DNA, 1350 bp, synthetic construct
  misc_feature 1..1350: note = antibody variant
  source 1..1350: mol_type = other DNA
cacgtccaattggtgcagtctggaggtggggtgaagaagattggggccgctgtgaggatctcctgcgaggtgactggatataaattcatggaccaactcataaactgggtgcggcaggcccccggtcagggccttgagtggatgggatggatgaatccaacatatggacaagtaaattattcatggagatttgaaggaagggtcaccatgaccagggacatggacaccgagacggccttcatggagttgagaggactgagagtggacgacacggccgtctattattgcgcgaggggaccctctggggaaaattatccttttcactattggggccagggtgtccgagtggtcgtctcgtcaccctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 295 - protein, 206 aa, synthetic construct; identical to SEQ ID NO: 36
  REGION 1..206: note = antibody variant

SEQ ID NO: 296 - DNA, 618 bp, synthetic construct; identical to SEQ ID NO: 112
  misc_feature 1..618: note = antibody variant
  source 1..618: mol_type = other DNA

SEQ ID NO: 297 - protein, 450 aa, synthetic construct; identical to SEQ ID NO: 293
  REGION 1..450: note = antibody variant

SEQ ID NO: 298 - DNA, 1350 bp, synthetic construct; identical to SEQ ID NO: 294
  misc_feature 1..1350: note = antibody variant
  source 1..1350: mol_type = other DNA

SEQ ID NO: 299 - protein, 206 aa, synthetic construct
  REGION 1..206: note = antibody variant
ASALTQPASMSASPGQSVTISCSGTRHIISAWFQQYPGKPPKLIIFDDDKRPSGVPSRFSASRPGDTASLTISNVQPEDEATYICNTYEFFGGGTRLTVLSQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 300 - DNA, 618 bp, synthetic construct
  misc_feature 1..618: note = antibody variant
  source 1..618: mol_type = other DNA
gcatctgccctgactcagcctgcctccatgtctgcgtcccctggacagtcggtaaccatctcgtgctctggaaccagacacataatctctgcttggttccaacaatatccaggcaaaccacccaaactcataatttttgacgacgataagcgtccctctggagttcctagtcgcttctctgcctccaggcctggcgacacggcctccctgacaatctctaatgttcaacctgaggacgaggcgacgtacatttgcaatacatatgaattctttggcggagggaccagattgaccgtcctaagtcagcccaaggctgccccctcggtcactctgttcccgccctcctctgaggagcttcaagccaacaaggccacactggtgtgtctcataagtgacttctacccgggagccgtgacagtggcttggaaagcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 301 - protein, 450 aa, synthetic construct
  REGION 1..450: note = antibody variant
HVQLVQSGGGVKKIGAAVRISCEVTGYKFMDQLINWVRQAPGQGLEWMGWMNPTYGQVNYSWRFEGRVTMTRDMDTETAFMELRGLRVDDTAVYYCARGPSGENYPFHYWGQGVRVVVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 302 - DNA, 1350 bp, synthetic construct
  misc_feature 1..1350: note = antibody variant
  source 1..1350: mol_type = other DNA
cacgtccaattggtgcagtctggaggtggggtgaagaagattggggccgctgtgaggatctcctgcgaggtgactggatataaattcatggaccaactcataaactgggtgcggcaggcccccggtcagggccttgagtggatgggatggatgaatccaacatatggacaagtaaattattcatggagatttgaaggaagggtcaccatgaccagggacatggacaccgagacggccttcatggagttgagaggactgagagtggacgacacggccgtctattattgcgcgaggggaccctctggggaaaattatccttttcactattggggccagggtgtccgagtggtcgtctcgtcatcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 303 - protein, 206 aa, synthetic construct
  REGION 1..206: note = antibody variant
ASALTQPASMSASPGQSVTISCSGTRHIISAWFQQYPGKPPKLIIFDDDKRPSGVPSRFSASRPGDTASLTISNVQPEDEATYICNTYEFFGGGTRLTVLGQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 304 - DNA, 618 bp, synthetic construct
  misc_feature 1..618: note = antibody variant
  source 1..618: mol_type = other DNA
gcatctgccctgactcagcctgcctccatgtctgcgtcccctggacagtcggtaaccatctcgtgctctggaaccagacacataatctctgcttggttccaacaatatccaggcaaaccacccaaactcataatttttgacgacgataagcgtccctctggagttcctagtcgcttctctgcctccaggcctggcgacacggcctccctgacaatctctaatgttcaacctgaggacgaggcgacgtacatttgcaatacatatgaattctttggcggagggaccagattgaccgtcctaggtcagcccaaggctgccccctcggtcactctgttcccaccctcctctgaggagcttcaagccaacaaggccacactggtgtgtctcgtaagtgacttctacccgggagccgtgacagtggcctggaaggcagatggcagccccgtcaaggtgggagtggagaccaccaaaccctccaaacaaagcaacaacaagtatgcggccagcagctacctgagcctgacgcccgagcagtggaagtcccacagaagctacagctgccgggtcacgcatgaagggagcaccgtggagaagacagtggcccctgcagaatgctct

SEQ ID NO: 305 - protein, 453 aa, synthetic construct
  REGION 1..453: note = antibody variant
HVQLVQSGGGVKKIGAAVRISCEVTGYKFMDQLINWVRQAPGQGLEWMGWMNPTYGQVNYSWRFEGRVTMTRDMDTETAFMELRGLRVDDTAVYYCVRDRSNGSGKRFESSNWGQGVRVVVSSPSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 306 - DNA, 1359 bp, synthetic construct
  misc_feature 1..1359: note = antibody variant
  source 1..1359: mol_type = other DNA
cacgtccaattggtgcagtctggaggtggggtgaagaagattggggccgctgtgaggatctcctgcgaggtgactggatataaattcatggaccaactcataaactgggtgcggcaggcccccggtcagggccttgagtggatgggatggatgaatccaacatatggacaagtaaattattcatggagatttgaaggaagggtcaccatgaccagggacatggacaccgagacggccttcatggagttgagaggactgagagtggacgacacggccgtctattattgcgtgagagatcgcagtaatggatcgggaaagcgattcgagtcctccaattggggccagggtgtccgagtggtcgtctcgtcaccctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 307 - protein, 206 aa, synthetic construct; identical to SEQ ID NO: 36
  REGION 1..206: note = antibody variant

SEQ ID NO: 308 - DNA, 618 bp, synthetic construct; identical to SEQ ID NO: 112
  misc_feature 1..618: note = antibody variant
  source 1..618: mol_type = other DNA

SEQ ID NO: 309 - protein, 458 aa, synthetic construct
  REGION 1..458: note = antibody variant
HVQLVQSGGGVKKIGAAVRISCEVTGYKFMDQLINWVRQAPGQGLEWMGWMNPTYGQVNYSWRFEGRVTMTRDMDTETAFMELRGLRVDDTAVYYCVRDRSNGSGKRFESSNWFLDLWGQGVRVVVSSPSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 310 - DNA, 1374 bp, synthetic construct
  misc_feature 1..1374: note = antibody variant
  source 1..1374: mol_type = other DNA
cacgtccaattggtgcagtctggaggtggggtgaagaagattggggccgctgtgaggatctcctgcgaggtgactggatataaattcatggaccaactcataaactgggtgcggcaggcccccggtcagggccttgagtggatgggatggatgaatccaacatatggacaagtaaattattcatggagatttgaaggaagggtcaccatgaccagggacatggacaccgagacggccttcatggagttgagaggactgagagtggacgacacggccgtctattattgcgtgagagatcgcagtaatggatcgggaaagcgattcgagtcctccaattggttcctcgatctgtggggccagggtgtccgagtggtcgtctcgtcaccctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 311 - protein, 206 aa, synthetic construct; identical to SEQ ID NO: 36
  REGION 1..206: note = antibody variant

SEQ ID NO: 312 - DNA, 618 bp, synthetic construct; identical to SEQ ID NO: 112
  misc_feature 1..618: note = antibody variant
  source 1..618: mol_type = other DNA

SEQ ID NO: 313 - protein, 458 aa, synthetic construct
  REGION 1..458: note = antibody variant
HVQLVQSGGGVKKIGAAVRISCEVTGYKFMDQLINWVRQAPGQGLEWMGWMNPTYGQVNYSWRFEGRVTMTRDMDTETAFMELRGLRVDDTAVYYCVRDRSNGSGKRFESSNWFLDLWGRGTAVTIQSSSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 314 - DNA, 1374 bp, synthetic construct
  misc_feature 1..1374: note = antibody variant
  source 1..1374: mol_type = other DNA
cacgtccaattggtgcagtctggaggtggggtgaagaagattggggccgctgtgaggatctcctgcgaggtgactggatataaattcatggaccaactcataaactgggtgcggcaggcccccggtcagggccttgagtggatgggatggatgaatccaacatatggacaagtaaattattcatggagatttgaaggaagggtcaccatgaccagggacatggacaccgagacggccttcatggagttgagaggactgagagtggacgacacggccgtctattattgcgtgagagatcgcagtaatggatcgggaaagcgattcgagtcctccaattggttcctcgatctgtggggccgtgggactgcggtcacaattcaatcatcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 315 - protein, 206 aa, synthetic construct
  REGION 1..206: note = antibody variant
ASALTQPASMSASPGQSVTISCSGTRHIISAWFQQYPGKPPKLIIFDDDKRPSGVPSRFSASRPGDTASLTISNVQPEDEATYICNTYEFFGGGTRLTVLRQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 316 - DNA, 618 bp, synthetic construct
  misc_feature 1..618: note = antibody variant
  source 1..618: mol_type = other DNA
gcatctgccctgactcagcctgcctccatgtctgcgtcccctggacagtcggtaaccatctcgtgctctggaaccagacacataatctctgcttggttccaacaatatccaggcaaaccacccaaactcataatttttgacgacgataagcgtccctctggagttcctagtcgcttctctgcctccaggcctggcgacacggcctccctgacaatctctaatgttcaacctgaggacgaggcgacgtacatttgcaatacatatgaattctttggcggagggaccagattgaccgtcctacgtcagcccaaggctgccccctcggtcactctgttcccaccctcctctgaggagcttcaagccaacaaggccacactggtgtgtctcgtaagtgacttctacccgggagccgtgacagtggcctggaaggcagatggcagccccgtcaaggtgggagtggagaccaccaaaccctccaaacaaagcaacaacaagtatgcggccagcagctacctgagcctgacgcccgagcagtggaagtcccacagaagctacagctgccgggtcacgcatgaagggagcaccgtggagaagacagtggcccctgcagaatgctct

SEQ ID NO: 317 - protein, 450 aa, synthetic construct
  REGION 1..450: note = antibody variant
HIQLLQSGPQVKKSGDTVRISCETSGYNFVDSRIHWVRQTPEKRLRWMGWINPLQGGVNYAPEFQGRIRMTRDTFIDTVYVDLSGLTPADTAYYYCARGIDGKSYPFHFWGHGTRVTVFSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 318 - DNA, 1350 bp, synthetic construct
  misc_feature 1..1350: note = antibody variant
  source 1..1350: mol_type = other DNA
cacattcagttgctacagtcggggcctcaagttaagaagtctggggacacagtgagaatctcctgtgagacctctggatataacttcgtcgactcccgtatccactgggtccgacagaccccggaaaaacgtctcagatggatgggctggatcaatcctctccaaggtggtgtgaattacgcgccggaatttcagggcagaatcaggatgaccagggacacatttatagacacagtttacgtggacctgagcggactgacaccggccgacacggcctattattactgcgcgcgagggatcgatggcaagtcttacccctttcatttctggggccacggaacccgggtcaccgtcttctcggcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 319 - protein, 203 aa, synthetic construct; identical to SEQ ID NO: 38
  REGION 1..203: note = antibody variant

SEQ ID NO: 320 - DNA, 609 bp, synthetic construct; identical to SEQ ID NO: 114
  misc_feature 1..609: note = antibody variant
  source 1..609: mol_type = other DNA

SEQ ID NO: 321 - protein, 453 aa, synthetic construct
  REGION 1..453: note = antibody variant
QVRLVQSGAGARKTGASMKLSCSTSGYTFTTHHGHFINWVRQARGQGLEWMGWMNPMTGQMNIEGKFQGRVTLTRDIYSDTAYMEMTRLTTGDTGTYYCARGDFGQNYPFHYWGQGSLVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 322 - DNA, 1359 bp, synthetic construct
  misc_feature 1..1359: note = antibody variant
  source 1..1359: mol_type = other DNA
caggtgcgcttggtgcagtctggggctggggcgaggaagactggggcctcaatgaaactttcctgctcgacctctggatacaccttcaccactcatcacggccacttcataaattgggtgcgacaggcccgtggacaagggcttgagtggatggggtggatgaatcccatgactgggcagatgaatattgaggggaaatttcagggcagagtcaccctcactcgagacatatacagtgacacggcttacatggaaatgaccagactgacaactggcgacacgggcacttattactgtgcgcgaggcgatttcggacagaattatccctttcattattggggccagggaagcctggtcatcgtctcctcggcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 323 - protein, 206 aa, synthetic construct; identical to SEQ ID NO: 40
  REGION 1..206: note = antibody variant

SEQ ID NO: 324 - DNA, 618 bp, synthetic construct; identical to SEQ ID NO: 116
  misc_feature 1..618: note = antibody variant
  source 1..618: mol_type = other DNA

SEQ ID NO: 325 - protein, 450 aa, synthetic construct
  REGION 1..450: note = antibody variant
HVQLVQSGGGVKKIGAAVRISCEVSGYNFMDQFINWVRQAPGQGLEWMGWMNPIYGQVNYSWRFQGRVTMTRDMYTDTAFMELRGLRVDDTAVYYCARGPSGENYPFHYWGQGVRVVVSSPSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 326 - DNA, 1350 bp, synthetic construct
  misc_feature 1..1350: note = antibody variant
  source 1..1350: mol_type = other DNA
cacgtccaattggtgcagtctggaggtggggtgaagaagattggggccgctgtgaggatctcctgcgaggtgtctggatacaacttcatggaccaattcataaattgggtgcgacaggcccccggtcagggccttgagtggatgggatggatgaacccaatatatggacaagtaaattattcatggagatttcaaggaagggtcaccatgaccagggacatgtacaccgacacggccttcatggagttgagaggactgagagtggacgacacggccgtctattattgcgcgaggggaccctctggggaaaattatccttttcactattggggccagggtgtccgagtggtcgtctcgtcaccctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 327 - protein, 203 aa, synthetic construct
  REGION 1..203: note = antibody variant
LTQPASMSASPGQSVTISCSGTRHIISAWFQQYPGKPPKLIIFDDDKRPSGVPSRFSASRPGDTASLTISNVQPEDEATYICNTYEFFGGGTKLTVLRQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 328 - DNA, 609 bp, synthetic construct
  misc_feature 1..609: note = antibody variant
  source 1..609: mol_type = other DNA
ctgactcagcctgcctccatgtctgcgtcccctggacagtcggtaaccatctcgtgctctggaaccagacacataatctctgcttggttccaacaatatccaggcaaaccacccaaactcataatttttgacgacgataagcgtccctctggagttcctagtcgcttctctgcctccaggcctggcgacacggcctccctgacaatctctaatgttcaacctgaggacgaggcgacatacatttgcaatacatatgaattctttggcggagggaccaaattgaccgtcctacgtcagcccaaggctgccccctcggtcactctgttcccaccctcctctgaggagcttcaagccaacaaggccacactggtgtgtctcgtaagtgacttctacccgggagccgtgacagtggcctggaaggcagatggcagccccgtcaaggtgggagtggagaccaccaaaccctccaaacaaagcaacaacaagtatgcggccagcagctacctgagcctgacgcccgagcagtggaagtcccacagaagctacagctgccgggtcacgcatgaagggagcaccgtggagaagacagtggcccctgcagaatgctct

SEQ ID NO: 329 - protein, 450 aa, synthetic construct; identical to SEQ ID NO: 325
  REGION 1..450: note = antibody variant

SEQ ID NO: 330 - DNA, 1350 bp, synthetic construct; identical to SEQ ID NO: 326
  misc_feature 1..1350: note = antibody variant
  source 1..1350: mol_type = other DNA

SEQ ID NO: 331 - protein, 203 aa, synthetic construct; identical to SEQ ID NO: 42
  REGION 1..203: note = antibody variant

SEQ ID NO: 332 - DNA, 609 bp, synthetic construct; identical to SEQ ID NO: 118
  misc_feature 1..609: note = antibody variant
  source 1..609: mol_type = other DNA

SEQ ID NO: 333 - protein, 450 aa, synthetic construct
  REGION 1..450: note = antibody variant
HIQLLQSGPQVKKSGDTVRISCETSGYNFVDSRIHWVRQTPEKRLRWMGWINPLHGGVNYAPEFQGRIRMTRDTFIDTVYVDLSGLTPADTAYYYCARGIDGKSYPFHFWGHGTRVTVFSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 334 - DNA, 1350 bp, synthetic construct
  misc_feature 1..1350: note = antibody variant
  source 1..1350: mol_type = other DNA
cacattcagttgctacagtcggggcctcaagttaagaagtctggggacacagtgagaatctcctgtgagacctctggatataacttcgtcgactcccgtatccactgggtccgacagaccccggaaaaacgtctcagatggatgggctggatcaatcctctccacggtggtgtgaattacgcgccggaatttcagggcagaatcaggatgaccagggacacatttatagacacagtttacgtggacctgagcggactgacaccggccgacacggcctattattactgcgcgcgagggatcgatggcaagtcttacccctttcatttctggggccacggaacccgggtcaccgtcttctcggcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 335 - protein, 203 aa, synthetic construct; identical to SEQ ID NO: 38
  REGION 1..203: note = antibody variant

SEQ ID NO: 336 - DNA, 609 bp, synthetic construct; identical to SEQ ID NO: 120
  misc_feature 1..609: note = antibody variant
  source 1..609: mol_type = other DNA

SEQ ID NO: 337 - protein, 455 aa, synthetic construct
  REGION 1..455: note = antibody variant
RVTLQQSGAIVRQPGASVTVSCETSGYTFTKYFIYWVRQAPGQGLEWLGRIHPRTGAVKYAPRFQGRLSMTRDWSLDTAYLGLTGLTLGDTALYFCARGAFEADSYGSSYPFHHWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 338 - DNA, 1365 bp, synthetic construct
  misc_feature 1..1365: note = antibody variant
  source 1..1365: mol_type = other DNA
cgtgttacattacaacaatctggggctatagtgaggcagcctggggcctcagtgaccgtctcctgcgagacttctggatatactttcaccaagtatttcatctactgggtgcgacaggcccctggacagggtcttgagtggctgggcagaatacacccccgaaccggtgccgtgaagtatgcaccgagatttcagggtagactgtccatgaccagagactggtcactcgacacagcctacctcggattgaccggactgacactcggcgacacggctctatatttctgtgcgaggggggcctttgaggcagattcatatgggtcaagttatccctttcaccactggggccagggaaccctagtcaccgtctccgcggcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 339 - protein, 207 aa, synthetic construct; identical to SEQ ID NO: 46
  REGION 1..207: note = antibody variant

SEQ ID NO: 340 - DNA, 621 bp, synthetic construct; identical to SEQ ID NO: 122
  misc_feature 1..621: note = antibody variant
  source 1..621: mol_type = other DNA

SEQ ID NO: 341 - protein, 455 aa, synthetic construct
  REGION 1..455: note = antibody variant
RVTLQQSGATVKQPGASVTVSCETSGYTFTKYTIHWVRQAPGQGLQWVGRIHPRTGAVKYAPIFQGKVSMSRDLSRDTAYLGLTRLTLADTALFFCARGAFEADLSGPTYPFHHWGQGTLVIVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 342 - DNA, 1365 bp, synthetic construct
  misc_feature 1..1365: note = antibody variant
  source 1..1365: mol_type = other DNA
cgtgtgacattacaacaatctggggctacagtgaagcagcctggggcctcagtgaccgtctcctgcgagacttctggatacactttcaccaagtataccattcactgggtgcgacaggcccctggacagggtcttcagtgggtgggcagaatacacccccgaaccggtgccgtgaagtatgcaccgatatttcagggtaaagtgtccatgagtcgagacttgtcacgcgacacagcctacctcggattgaccagactgacgctcgccgacacggctctatttttctgtgcgaggggggcctttgaggcagatttaagtgggccaacttacccctttcaccactggggccaaggaaccctagtcatcgtctccgcggcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 343 - protein, 207 aa, synthetic construct; identical to SEQ ID NO: 48
  REGION 1..207: note = antibody variant

SEQ ID NO: 344 - DNA, 621 bp, synthetic construct; identical to SEQ ID NO: 124
  misc_feature 1..621: note = antibody variant
  source 1..621: mol_type = other DNA

SEQ ID NO: 345 - protein, 450 aa, synthetic construct
  REGION 1..450: note = antibody variant
NVQLMQSGTEVKKSGASVTISCETAGFNFIDSVIHWLRQAPGGGFQWMGWIKPLRGAVNYPQFLQGRVSMTRDLSTDTVYMVLNGLTPDDTGLYYCAKGAFRGGSPFGFWGQGTLLTVSPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 346 - DNA, 1350 bp, synthetic construct
  misc_feature 1..1350: note = antibody variant
  source 1..1350: mol_type = other DNA
aacgtgcagttgatgcagtctgggactgaggtgaagaagtctggggcctcggtgacaatctcttgtgagaccgctggattcaacttcatcgactccgtcatacactggctgcgccaggcccctggaggaggatttcagtggatggggtggatcaagcctcttagaggtgccgtcaattatccacagtttttgcagggcagggtctccatgacccgggacttgtccaccgacacggtgtacatggtcttgaatggactgacacctgacgacacaggcctttattactgcgcgaaaggggcctttagagggggttctccctttggcttctggggccagggaactctgctcaccgtctccccagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 347 - protein, 202 aa, synthetic construct; identical to SEQ ID NO: 50
  REGION 1..202: note = antibody variant

SEQ ID NO: 348 - DNA, 606 bp, synthetic construct; identical to SEQ ID NO: 126
  misc_feature 1..606: note = antibody variant
  source 1..606: mol_type = other DNA

SEQ ID NO: 349 - protein, 450 aa, synthetic construct
  REGION 1..450: note = antibody variant
QVRLVQSGPQVKKTGASVRVSCETSGYTFTSYFIHWLRLGPGEGLQWMGWINPLHGAVNYENKFRGRVTITRDTSTDTVYLDMSRLTPDDTAVYFCTRGIVADGWPYGHWGQGTQVTVSPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 350 - DNA, 1350 bp, synthetic construct
  misc_feature 1..1350: note = antibody variant
  source 1..1350: mol_type = other DNA
caggtgcgactggtgcagtctgggcctcaggtgaagaagactggggcctcagtgagggtctcctgcgaaacctctggatacacgttcacctcctacttcatccattggttacgactgggccccggagaggggcttcagtggatggggtggatcaaccctttacatggtgccgtgaattatgaaaacaaatttaggggcagggtcacaatcaccagggacacgtccacagacacagtgtatttggacatgagcagactgacccctgacgacacggccgtctatttctgcacaagaggaatcgttgctgatgggtggccctatggccactggggccagggaacccaagtcaccgtctccccggcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 351 - protein, 203 aa, synthetic construct; identical to SEQ ID NO: 52
  REGION 1..203: note = antibody variant

SEQ ID NO: 352 - DNA, 609 bp, synthetic construct; identical to SEQ ID NO: 128
  misc_feature 1..609: note = antibody variant
  source 1..609: mol_type = other DNA

SEQ ID NO: 353 - protein, 451 aa, synthetic construct
  REGION 1..451: note = antibody variant
QVRLQQSGVVVRKPGASVRISCETSGFTFIDHIVHWVRRAPGRGFEWMGWIKPLRGAVDYAPQLRGRISLTRDIYSETVFIDVSRLTSGDTAIYFCCKAAAPEEAFPLQYWGQGTQLIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 354 - DNA, 1353 bp, synthetic construct
  misc_feature 1..1353: note = antibody variant
  source 1..1353: mol_type = other DNA
caggtgcgacttcagcagtctggtgtcgtggtgaggaagcctggggcctcagtgagaatttcctgcgagacttctggattcaccttcatcgaccacattgtccattgggtgcggcgggcccctggacgaggctttgaatggatgggttggatcaagcctcttaggggtgccgtagattatgcaccccaacttcggggcaggatctccctgacgagggacatttacagtgaaaccgtctttatagacgtgagccgactgacgtctggcgacacggcgatatacttttgttgtaaggccgccgcccctgaagaagcattcccccttcaatactggggccaggggacccaacttatcgtctcctcggcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 355 - protein, 205 aa, synthetic construct; identical to SEQ ID NO: 54
  REGION 1..205: note = antibody variant

SEQ ID NO: 356 - DNA, 615 bp, synthetic construct; identical to SEQ ID NO: 130
  misc_feature 1..615: note = antibody variant
  source 1..615: mol_type = other DNA

SEQ ID NO: 357 - protein, 451 aa, synthetic construct
  REGION 1..451: note = antibody variant
QVRLQQSGVVVRKPGASVRLSCETSGFKFIDHIVNWVRRAPGRGFEWMGWIKPLGGVADYAPQHRGRISLTRDIYTETVFIDLSRLTSGDTAIYFCCKAAAPDEAFPLEYWGQGTQLIVSPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 358 - DNA, 1353 bp, synthetic construct
  misc_feature 1..1353: note = antibody variant
  source 1..1353: mol_type = other DNA
caggtgcgacttcagcagtctggtgtcgtggtgaggaagcctggggcctcagtgagactttcctgcgagacgtctggattcaaattcatcgaccacattgtcaactgggtgcggcgggcccctggacgaggctttgaatggatgggttggatcaagcctcttgggggtgtcgctgattatgcaccccaacatcggggcaggatctcactgacgagggacatttacactgaaaccgtctttatagacctgagtcgactgacgtctggcgacacggcgatttatttctgttgtaaggccgccgcccctgatgaagcattcccccttgaatactggggccaggggacccaacttatcgtctccccggcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 359 - protein, 205 aa, synthetic construct; identical to SEQ ID NO: 56
  REGION 1..205: note = antibody variant

SEQ ID NO: 360 - DNA, 615 bp, synthetic construct; identical to SEQ ID NO: 132
  misc_feature 1..615: note = antibody variant
  source 1..615: mol_type = other DNA

SEQ ID NO: 361 - protein, 451 aa, synthetic construct
  REGION 1..451: note = antibody variant
QVRLVQSGPVMRKPGASVRISCETSGFAFLDHIVHWVRRAPGRGFEWMGWVKTIGGVVDYAPHLRGRISVTRDVFSETVFLDLSRLTSGDTAMYFCSKAAAPDEAFPLEFWGQGTQVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 362 - DNA, 1353 bp, synthetic construct
  misc_feature 1..1353: note = antibody variant
  source 1..1353: mol_type = other DNA
caggtgcgacttgtgcagtctggtcccgtgatgagaaagcctggggcctcagtgagaatttcttgcgagacatctggattcgccttcttggaccacattgtccactgggtgcggcgggcccctggacgcggctttgaatggatgggttgggttaagaccattgggggtgtcgttgattatgcaccccaccttaggggcaggatctccgtgacgagagacgtctttagtgaaaccgtctttctggacttgagccgactgacgtctggcgacacggcgatgtatttttgttctaaggccgccgcccctgacgaagccttcccccttgaattttggggccaggggacccaagtcatcgtctcctcggcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 363 - protein, 205 aa, synthetic construct; identical to SEQ ID NO: 58
  REGION 1..205: note = antibody variant

SEQ ID NO: 364 - DNA, 615 bp, synthetic construct; identical to SEQ ID NO: 134
  misc_feature 1..615: note = antibody variant
  source 1..615: mol_type = other DNA

SEQ ID NO: 365 - protein, 451 aa, synthetic construct
  REGION 1..451: note = antibody variant
QVRLVQSGPVVRKPGTSVRISCETSGFAFLDHIVHWVRRAPGRGFEWMGWVKTIGGVVDYAPHLRGRISVTRDVFSEIVFMELSRLTSGDTAMYFCSKAAAPDEAFPLEFWGQGTQVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 366 - DNA, 1353 bp, synthetic construct
  misc_feature 1..1353: note = antibody variant
  source 1..1353: mol_type = other DNA
caggtgcgacttgtgcagtctggtcccgtggtgagaaagcctgggacctcagtgagaatttcttgcgagacatctggattcgccttcttggaccacattgtccactgggtgcggcgggcccctggacgcggctttgaatggatgggttgggttaagaccattgggggtgtcgttgattatgcaccccaccttaggggcaggatctccgtgacgagggacgtatttagtgaaatcgtctttatggagttgagtcgactgacgtctggcgacacggcgatgtatttttgttctaaggccgccgcccctgacgaagccttcccccttgaattttggggccaggggacccaagtcatcgtctcctcggcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 367 - protein, 205 aa, synthetic construct; identical to SEQ ID NO: 60
  REGION 1..205: note = antibody variant

SEQ ID NO: 368 - DNA, 615 bp, synthetic construct; identical to SEQ ID NO: 136
  misc_feature 1..615: note = antibody variant
  source 1..615: mol_type = other DNA

SEQ ID NO: 369 - protein, 450 aa, synthetic construct
  REGION 1..450: note = antibody variant
QVQLVQSGAGVKKPGASVRVSCETSGFKFTEYFIHFLRQAPGQGLEWMGWLNPLRGAVNYPRKFQGRVTLTRDIYTTTVYMQLNGLTPDDTAVYYCARAVFNEAFPFDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 370 - DNA, 1350 bp, synthetic construct
  misc_feature 1..1350: note = antibody variant
  source 1..1350: mol_type = other DNA
caagtgcaactggtgcagtctggggctggggtgaagaagcctggggcctcagtgagggtctcctgcgagacatccggattcaagttcaccgagtactttatccactttttacgacaggcccctggacaagggcttgagtggatgggatggctcaaccctctcagaggtgccgtcaactatccacggaagtttcagggcagagtcactttgaccagggacatctacaccaccaccgtctacatgcaacttaacggtctgacccctgacgacacggccgtctactactgtgccagagcggtctttaatgaagctttcccctttgactactggggccagggaagcctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 371 - protein, 204 aa, synthetic construct; identical to SEQ ID NO: 62
  REGION 1..204: note = antibody variant

SEQ ID NO: 372 - DNA, 612 bp, synthetic construct; identical to SEQ ID NO: 138
  misc_feature 1..612: note = antibody variant
  source 1..612: mol_type = other DNA

SEQ ID NO: 373 - protein, 451 aa, synthetic construct; identical to SEQ ID NO: 357
  REGION 1..451: note = antibody variant

SEQ ID NO: 374 - DNA, 1353 bp, synthetic construct
  misc_feature 1..1353: note = antibody variant
  source 1..1353: mol_type = other DNA
caggtgcgacttcagcagtctggtgtcgtggtgaggaagcctggggcctcagtgagactttcctgcgagacgtctggcttcaaattcatcgaccacattgtcaactgggtgcggcgggcccctggacgaggctttgaatggatgggttggatcaagcctcttgggggtgtcgctgattatgcaccccaacatcggggcaggatctcactgacgagggacatttacactgaaaccgtctttatagacctgagtcgactgacgtctggcgacacggcgatttatttttgttgtaaggccgccgcccctgatgaagcattcccccttgaatactggggccaggggacccaacttatcgtctccccggcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 375 - protein, 205 aa, synthetic construct; identical to SEQ ID NO: 64
  REGION 1..205: note = antibody variant

SEQ ID NO: 376 - DNA, 615 bp, synthetic construct; identical to SEQ ID NO: 140
  misc_feature 1..615: note = antibody variant
  source 1..615: mol_type = other DNA

SEQ ID NO: 377 - protein, 451 aa, synthetic construct; identical to SEQ ID NO: 361
  REGION 1..451: note = antibody variant

SEQ ID NO: 378 - DNA, 1353 bp, synthetic construct
  misc_feature 1..1353: note = antibody variant
  source 1..1353: mol_type = other DNA
caggtgcgacttgtgcagtctggtcccgtgatgagaaagcctggggcctcagtgagaatttcttgcgagacatctggattcgccttcttggaccacattgtccactgggtgcggcgggcccctggacgcggctttgaatggatgggttgggttaagaccattgggggtgtcgttgattatgcaccccaccttaggggcaggatctccgtgacgagagacgtctttagtgaaaccgtctttctggacttgagtcgactgacgtctggcgacacggcgatgtatttttgttctaaggccgccgcccctgacgaagccttcccccttgaattttggggccaggggacccaagtcatcgtctcctcggcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 379 - protein, 205 aa, synthetic construct; identical to SEQ ID NO: 58
  REGION 1..205: note = antibody variant

SEQ ID NO: 380 - DNA, 615 bp, synthetic construct; identical to SEQ ID NO: 142
  misc_feature 1..615: note = antibody variant
  source 1..615: mol_type = other DNA

SEQ ID NO: 381 - protein, 450 aa, synthetic construct
  REGION 1..450: note = antibody variant
HVQLRQSGTEAKKSGASVTISCETAGFNFIDSVIHWLRQAPGGGFQWMGWIKPLRGGVNYPHYLQGRISMTRDLSSDTVYMVLNRLTPADTGLYYCAKGAFGGSSPFGFWGQGTLLTVSPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 382 - DNA, 1350 bp, synthetic construct
  misc_feature 1..1350: note = antibody variant
  source 1..1350: mol_type = other DNA
cacgtgcagttgaggcagtctgggactgaggcgaagaagtctggggcctcggtgacaatctcttgtgagaccgctggattcaacttcatcgactccgtcatacactggctgcgccaggcccctggtgggggatttcagtggatggggtggatcaagcctcttagaggtggcgtcaattatccacattatttgcagggcagaatctccatgacccgggacttgtccagtgacacggtttacatggtcttaaatagactgacacctgccgacacaggcctttattactgcgcgaaaggggcctttggggggagttctccctttggcttctggggccagggaactctgctcaccgtctccccagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 383 - protein, 202 aa, synthetic construct; identical to SEQ ID NO: 68
  REGION 1..202: note = antibody variant

SEQ ID NO: 384 - DNA, 606 bp, synthetic construct; identical to SEQ ID NO: 144
  misc_feature 1..606: note = antibody variant
  source 1..606: mol_type = other DNA

SEQ ID NO: 385 - protein, 450 aa, synthetic construct
  REGION 1..450: note = antibody variant
HVQLMQSGTQAKKSGASVTISCETAGFKFIDSVIHWLRQAPGGGFQWMGWIKPLGGAVNYPPYLQGRISLTRDLSTDTIYMVLNGLTPADTGFYYCAKGAFGGGSPFGFWGQGTLLTVSPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 386 - DNA, 1350 bp, synthetic construct
  misc_feature 1..1350: note = antibody variant
  source 1..1350: mol_type = other DNA
cacgtgcaattgatgcagtctgggactcaggcgaagaagtctggggcctcggtgacaatttcttgtgagaccgctggattcaagttcatcgactccgtcatacactggctgcgccaggcccctggagggggatttcagtggatggggtggatcaagcctcttggaggtgccgtcaactatccaccctatttgcagggcaggatctccttgacccgtgacttgtccaccgacacaatttacatggtcttgaatggactgacacctgccgacacaggcttttattactgcgccaaaggggcctttggggggggttctccctttggcttctggggccaggggactctgctcaccgtctccccagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 387 - protein, 202 aa, synthetic construct; identical to SEQ ID NO: 70
  REGION 1..202: note = antibody variant

SEQ ID NO: 388 - DNA, 606 bp, synthetic construct; identical to SEQ ID NO: 146
  misc_feature 1..606: note = antibody variant
  source 1..606: mol_type = other DNA

SEQ ID NO: 389 - protein, 450 aa, synthetic construct
  REGION 1..450: note = antibody variant
HVQLRQSGTEAKKSGASVTISCETAGFNFIDSVIHWLRQAPGGGFQWMGWIKPLRGGVNYPHYLQGRISMTRDLSSDTVYMVLNRLTPDDTGLYYCAKGAFGGSSPFGFWGQGTLLTVSPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 390 - DNA, 1350 bp, synthetic construct
  misc_feature 1..1350: note = antibody variant
  source 1..1350: mol_type = other DNA
cacgtgcagttgaggcagtctgggactgaggcgaagaagtctggggcctcggtgacaatctcttgtgagaccgctggattcaacttcatcgactccgtcatacactggctgcgccaggcccctggtgggggatttcagtggatggggtggatcaagcctcttagaggtggcgtcaattatccacattatttgcagggcagaatctccatgacccgggacttgtccagtgacacggtttacatggtcttaaatagactgacacctgacgacacaggcctttactactgcgcgaaaggggcctttggggggagttctccctttggcttctggggccagggaactctgctcaccgtctccccagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 391 - protein, 202 aa, synthetic construct; identical to SEQ ID NO: 72
  REGION 1..202: note = antibody variant

SEQ ID NO: 392 - DNA, 606 bp, synthetic construct; identical to SEQ ID NO: 148
  misc_feature 1..606: note = antibody variant
  source 1..606: mol_type = other DNA

SEQ ID NO: 393 - protein, 455 aa, synthetic construct
  REGION 1..455: note = antibody variant
RVTLQQSGATVRQPGASVTVSCETSGFTFIKYTIHWVRQAPGQGLQWVGRIHPRTGAVKFAPIFQGKFSMSRDLSRDTAYLGLTRLTLADTALFFCARGAFEADLYGPTYPFHHWGQGTQVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 394 - DNA, 1365 bp, synthetic construct
  misc_feature 1..1365: note = antibody variant
  source 1..1365: mol_type = other DNA
cgtgttacattacaacagtctggggctacagtgaggcagcctggggcctcagtcaccgtctcctgcgagacttctggattcaccttcatcaaatataccattcactgggtgcgacaggcccctggacagggtcttcagtgggtgggaagaatacacccccgaaccggtgccgtgaagtttgcaccgatatttcagggtaaattttccatgagtcgagacttgtcacgcgacacagcctacctcggattgaccagactgacactcgccgacacggctctatttttctgtgcgaggggggcctttgaggcagatttatatgggccaacttacccctttcaccactggggccaaggaacccaagtcaccgtctccgcggcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 395 - protein, 207 aa, synthetic construct; identical to SEQ ID NO: 74
  REGION 1..207: note = antibody variant

SEQ ID NO: 396 - DNA, 621 bp, synthetic construct; identical to SEQ ID NO: 150
  misc_feature 1..621: note = antibody variant
  source 1..621: mol_type = other DNA

SEQ ID NO: 397 - protein, 450 aa, synthetic construct
  REGION 1..450: note = antibody variant
HIQLLQSGPQVKKSGDTVRISCETSGYNFVDSLIHWVRQTPEKRLRWMGWINPLQGGVNYAPEFQGRIRMTRDTFIDTVYVDLSGLTPADTAYYYCARGIDGNSYPFHFWGHGTRVTVFSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 398 - DNA, 1350 bp, synthetic construct
  misc_feature 1..1350: note = antibody variant
  source 1..1350: mol_type = other DNA
cacattcagttgctacagtcggggcctcaagttaagaagtctggggacacagtgagaatctcctgtgagacctctggatataatttcgtcgactcccttatccactgggtccgacagaccccggaaaaacgtctcagatggatgggctggatcaatcctctccaaggtggtgtgaattacgcgccggaatttcagggcagaatcaggatgaccagggacacgtttatagacacagtttacgtggacttgagcggactgacaccggccgacacggcctattattactgcgcgcgagggatcgatggcaattcttacccctttcatttctggggccacggaacccgggtcaccgtcttctcggcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 399 - protein, 203 aa, synthetic construct; identical to SEQ ID NO: 76
  REGION 1..203: note = antibody variant

SEQ ID NO: 400 - DNA, 609 bp, synthetic construct; identical to SEQ ID NO: 152
  misc_feature 1..609: note = antibody variant
  source 1..609: mol_type = other DNA

SEQ ID NO: 401 - protein, 8 aa, synthetic construct
  REGION 1..8: note = CDR
GYDFIDYV

SEQ ID NO: 402 - protein, 8 aa, synthetic construct
  REGION 1..8: note = CDR
MNPSGGGT

SEQ ID NO: 403 - protein, 22 aa, synthetic construct
  REGION 1..22: note = CDR
VRDRANGSGRRRFESVNWFLDL

SEQ ID NO: 404 - protein, 5 aa, synthetic construct
  REGION 1..5: note = CDR
WAFEN

SEQ ID NO: 405 - protein, 8 aa, synthetic construct
  REGION 1..8: note = CDR
GYKFPDYI

SEQ ID NO: 406 - protein, 8 aa, synthetic construct
  REGION 1..8: note = CDR
INPMGGQV

SEQ ID NO: 407 - protein, 16 aa, synthetic construct
  REGION 1..16: note = CDR
VRDRSNGSGRRFESSN

SEQ ID NO: 408 - protein, 5 aa, synthetic construct
  REGION 1..5: note = CDR
WAYEA

SEQ ID NO: 409 - protein, 8 aa, synthetic construct
  REGION 1..8: note = CDR
GYTFTDYL

SEQ ID NO: 410 - protein, 8 aa, synthetic construct
  REGION 1..8: note = CDR
MNPVYGQV

SEQ ID NO: 411 - protein, 21 aa, synthetic construct
  REGION 1..21: note = CDR
VRDTGDGSRRHFDSINWFLDL

SEQ ID NO: 412 - protein, 5 aa, synthetic construct
  REGION 1..5: note = CDR
WAFEA

SEQ ID NO: 413 - protein, 8 aa, synthetic construct
  REGION 1..8: note = CDR
GYTFTDYV

SEQ ID NO: 414 - protein, 8 aa, synthetic construct
  REGION 1..8: note = CDR
IDPPYGQV

SEQ ID NO: 415 - protein, 21 aa, synthetic construct
  REGION 1..21: note = CDR
VRDRSNGWGKRFESSNWFLDL

SEQ ID NO: 416 - protein, 8 aa, synthetic construct
  REGION 1..8: note = CDR
GYTFVDYF

SEQ ID NO: 417 - protein, 8 aa, synthetic construct
  REGION 1..8: note = CDR
MDPLNGRP

SEQ ID NO: 418 - protein, 21 aa, synthetic construct
  REGION 1..21: note = CDR
VRDKSNGSGRRFDSSNWFLDL

SEQ ID NO: 419 - protein, 5 aa, synthetic construct
  REGION 1..5: note = CDR
WAYDA

SEQ ID NO: 420 - protein, 8 aa, synthetic construct
  REGION 1..8: note = CDR
GYTFSDYI

SEQ ID NO: 421 - protein, 8 aa, synthetic construct
  REGION 1..8: note = CDR
MNPMGGQV

SEQ ID NO: 422 - protein, 5 aa, synthetic construct
  REGION 1..5: note = CDR
WAYEV

SEQ ID NO: 423 - protein, 8 aa, synthetic construct
  REGION 1..8: note = CDR
GYTFIDYI

SEQ ID NO: 424 - protein, 8 aa, synthetic construct
  REGION 1..8: note = CDR
IDPMNGRP

SEQ ID NO: 425 - protein, 21 aa, synthetic construct
  REGION 1..21: note = CDR
VRDKSNGSGKRFDSSNWFLDL

SEQ ID NO: 426 - protein, 8 aa, synthetic construct
  REGION 1..8: note = CDR
GYTFTDYI

SEQ ID NO: 427 - protein, 8 aa, synthetic construct
  REGION 1..8: note = CDR
MNPMGGRT

SEQ ID NO: 428 - protein, 8 aa, synthetic construct
  REGION 1..8: note = CDR
GYTFVDYL

SEQ ID NO: 429 - protein, 8 aa, synthetic construct
  REGION 1..8: note = CDR
MDPMNGRP

SEQ ID NO: 430 - protein, 21 aa, synthetic construct
  REGION 1..21: note = CDR
VRDKSGGSGKLFDSSNWFLDL

SEQ ID NO: 431 - protein, 8 aa, synthetic construct
  REGION 1..8: note = CDR
INPGYGQV

SEQ ID NO: 432 - protein, 8 aa, synthetic construct
  REGION 1..8: note = CDR
MDPSYGQV

SEQ ID NO: 433 - protein, 21 aa, synthetic construct
  REGION 1..21: note = CDR
VRDRSHGSGRQFESSNWFLDL

SEQ ID NO: 434 - protein, 8 aa, synthetic construct
  REGION 1..8: note = CDR
MDPSFGQM

SEQ ID NO: 435 - protein, 21 aa, synthetic construct
  REGION 1..21: note = CDR
VRDRSHGSGRLFESSNWFLDL

SEQ ID NO: 436 - protein, 8 aa, synthetic construct
  REGION 1..8: note = CDR
GYRFTDYV

SEQ ID NO: 437 - protein, 8 aa, synthetic construct
  REGION 1..8: note = CDR
MDPSFGRM

SEQ ID NO: 438 - protein, 8 aa, synthetic construct
  REGION 1..8: note = CDR
GYTFIDYV

SEQ ID NO: 439 - protein, 8 aa, synthetic construct
  REGION 1..8: note = CDR
MDPTYGRM

SEQ ID NO: 440 - protein, 8 aa, synthetic construct
  REGION 1..8: note = CDR
GYRFLDYI

SEQ ID NO: 441 - protein, 21 aa, synthetic construct
  REGION 1..21: note = CDR
VRDRSNGSGKRFESSNWFLDL

SEQ ID NO: 442 - protein, 8 aa, synthetic construct
  REGION 1..8: note = CDR
GYKFMDQL

SEQ ID NO: 443 - protein, 8 aa, synthetic construct
  REGION 1..8: note = CDR
MNPTYGQV

SEQ ID NO: 444 - protein, 13 aa, synthetic construct
  REGION 1..13: note = CDR
ARGPSGENYPFHY

SEQ ID NO: 445 - protein, 4 aa, synthetic construct
  REGION 1..4: note = CDR
RHII

SEQ ID NO: 446 - protein, 5 aa, synthetic construct
  REGION 1..5: note = CDR
NTYEF

SEQ ID NO: 447 - protein, 8 aa, synthetic construct
  REGION 1..8: note = CDR
GYNFVDSR

SEQ ID NO: 448 - protein, 8 aa, synthetic construct
  REGION 1..8: note = CDR
INPLQGGV

SEQ ID NO: 449 - protein, 13 aa, synthetic construct
  REGION 1..13: note = CDR
ARGIDGKSYPFHF

SEQ ID NO: 450 - protein, 5 aa, synthetic construct
  REGION 1..5: note = CDR
SILEF

SEQ ID NO: 451 - protein, 8 aa, synthetic construct
  REGION 1..8: note = CDR
GYTFTKYF

SEQ ID NO: 452 - protein, 8 aa, synthetic construct
  REGION 1..8: note = CDR
IHPRTGAV

SEQ ID NO: 453 - protein, 18 aa, synthetic construct
  REGION 1..18: note = CDR
ARGAFEADSYGSSYPFHH

SEQ ID NO: 454 - protein, 5 aa, synthetic construct
  REGION 1..5: note = CDR
GNYNP

SEQ ID NO: 455 - protein, 5 aa, synthetic construct
  REGION 1..5: note = CDR
ASFEF

SEQ ID NO: 456 - protein, 8 aa, synthetic construct
  REGION 1..8: note = CDR
GYTFTKYT

SEQ ID NO: 457 - protein, 18 aa, synthetic construct
  REGION 1..18: note = CDR
ARGAFEADLSGPTYPFHH

SEQ ID NO: 458 - protein, 8 aa, synthetic construct
  REGION 1..8: note = CDR
GFNFIDSV

SEQ ID NO: 459 - protein, 8 aa, synthetic construct
  REGION 1..8: note = CDR
IKPLRGAV

SEQ ID NO: 460 - protein, 13 aa, synthetic construct
  REGION 1..13: note = CDR
AKGAFRGGSPFGF

SEQ ID NO: 461 - protein, 5 aa, synthetic construct
  REGION 1..5: note = CDR
ASREF

SEQ ID NO: 462 - protein, 8 aa, synthetic construct
  REGION 1..8: note = CDR
MNPMTGQM

SEQ ID NO: 463 - protein, 13 aa, synthetic construct
  REGION 1..13: note = CDR
ARGDFGQNYPFHY

SEQ ID NO: 464 - protein, 4 aa, synthetic construct
  REGION 1..4: note = CDR
NRYL

SEQ ID NO: 465 - protein, 5 aa, synthetic construct
  REGION 1..5: note = CDR
ASYER

SEQ ID NO: 466 - protein, 8 aa, synthetic construct
  REGION 1..8: note = CDR
GYNFMDQF

SEQ ID NO: 467 - protein, 8 aa, synthetic construct
  REGION 1..8: note = CDR
MNPIYGQV

SEQ ID NO: 468 - protein, 8 aa, synthetic construct
  REGION 1..8: note = CDR
INPLHGGV

SEQ ID NO: 469 - protein, 8 aa, synthetic construct
  REGION 1..8: note = CDR
GYTFTSYF

SEQ ID NO: 470 - protein, 8 aa, synthetic construct
  REGION 1..8: note = CDR
INPLHGAV

SEQ ID NO: 471 - protein, 13 aa, synthetic construct
  REGION 1..13: note = CDR
TRGIVADGWPYGH

SEQ ID NO: 472 - protein, 5 aa, synthetic construct
  REGION 1..5: note = CDR
SSLQF

SEQ ID NO: 473 - protein, 8 aa, synthetic construct
  REGION 1..8: note = CDR
GFTFIDHI

SEQ ID NO: 474 - protein, 14 aa, synthetic construct
  REGION 1..14: note = CDR
CKAAAPEEAFPLQY

SEQ ID NO: 475 - protein, 5 aa, synthetic construct
  REGION 1..5: note = CDR
SSRTF

SEQ ID NO: 476 - protein, 8 aa, synthetic construct
  REGION 1..8: note = CDR
GFKFIDHI

SEQ ID NO: 477 - protein, 8 aa, synthetic construct
  REGION 1..8: note = CDR
IKPLGGVA

SEQ ID NO: 478 - protein, 14 aa, synthetic construct
  REGION 1..14: note = CDR
CKAAAPDEAFPLEY

SEQ ID NO: 479 - protein, 5 aa, synthetic construct
  REGION 1..5: note = CDR
SSTTF

SEQ ID NO: 480 - protein, 7 aa, synthetic construct
  REGION 1..7: note = CDR
GFAFLDH

SEQ ID NO: 481 - protein, 8 aa, synthetic construct
  REGION 1..8: note = CDR
VKTIGGVV

SEQ ID NO: 482 - protein, 14 aa, synthetic construct
  REGION 1..14: note = CDR
SKAAAPDEAFPLEF

SEQ ID NO: 483 - protein, 8 aa, synthetic construct
  REGION 1..8: note = CDR
GFKFTEYF

SEQ ID NO: 484 - protein, 8 aa, synthetic construct
  REGION 1..8: note = CDR
LNPLRGAV

SEQ ID NO: 485 - protein, 13 aa, synthetic construct
  REGION 1..13: note = CDR
ARAVFNEAFPFDY

SEQ ID NO: 486 - protein, 8 aa, synthetic construct
  REGION 1..8: note = CDR
GFAFLDHI

SEQ ID NO: 487 - protein, 8 aa, synthetic construct
  REGION 1..8: note = CDR
GFKFIDSV

SEQ ID NO: 488 - protein, 8 aa, synthetic construct
  REGION 1..8: note = CDR
IKPLGGAV

SEQ ID NO: 489 - protein, 13 aa, synthetic construct
  REGION 1..13: note = CDR
AKGAFGGGSPFGF

SEQ ID NO: 490 - protein, 8 aa, synthetic construct
  REGION 1..8: note = CDR
IKPLRGGV

SEQ ID NO: 491 - protein, 13 aa, synthetic construct
  REGION 1..13: note = CDR
AKGAFGGSSPFGF

SEQ ID NO: 492 - protein, 8 aa, synthetic construct
  REGION 1..8: note = CDR
GFTFIKYT

SEQ ID NO: 493 - protein, 18 aa, synthetic construct
  REGION 1..18: note = CDR
ARGAFEADLYGPTYPFHH

SEQ ID NO: 494 - protein, 5 aa, synthetic construct
  REGION 1..5: note = CDR
GSYNP

SEQ ID NO: 495 - protein, 8 aa, synthetic construct
  REGION 1..8: note = CDR
GYNFVDSL

SEQ ID NO: 496 - protein, 13 aa, synthetic construct
  REGION 1..13: note = CDR
ARGIDGNSYPFHF

SEQ ID NO: 497 - protein, 8 aa, synthetic construct
  REGION 1..8: note = CDR
GYRFPDYI

SEQ ID NO: 498 - protein, 16 aa, synthetic construct
  REGION 1..16: note = CDR
VRDRSNGSGKRFESSN

SEQ ID NO: 499 - protein, 6 aa, synthetic construct
  REGION 1..6: note = CDR
NTYEFF

SEQ ID NO: 500 - protein, 11 aa, synthetic construct
  REGION 1..11: note = CDR
GYTFTTHHGHF

SEQ ID NO: 501 - protein, 464 aa, synthetic construct
  REGION 1..464: note = antibody heavy chain
ADLVQSGAVVKKPGDSVRISCEAQGYRFPDYIIHWIRRAPGQGPEWMGWMNPMGGQVNIPWKFQGRVSMTRQLSQDPDDPDWGTAFLDLRGLKSDDTAVYYCVRDRSNGSGKRFESSNWFLDLWGRGTAVTIQSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 502 - DNA, 1392 bp, synthetic construct
  misc_feature 1..1392: note = antibody heavy chain
  source 1..1392: mol_type = other DNA
gcggacttggtgcagtctggggctgtggtgaagaagcctggggactcagtgaggatctcctgtgaggctcaaggatatagatttcctgactacatcattcactggattcgacgggcccctggacaaggccctgaatggatgggatggatgaatccaatgggcggacaagtaaatattccatggaaatttcagggtagggtctccatgacccggcaattatctcaagacccagacgacccggactggggcacagcatttctggacttaagaggactaaagtctgacgacacggccgtctattattgcgtgagagatcgcagtaatggatcgggaaagcgattcgagtcctccaattggttcctcgatctgtggggccgtgggactgcggtcacaattcaatcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 503 - protein, 203 aa, synthetic construct; identical to SEQ ID NO: 163
  REGION 1..203: note = antibody light chain

SEQ ID NO: 504 - DNA, 609 bp, synthetic construct; identical to SEQ ID NO: 164
  misc_feature 1..609: note = antibody light chain
  source 1..609: mol_type = other DNA

SEQ ID NO: 505 - protein, 457 aa, synthetic construct
  REGION 1..457: note = antibody heavy chain
HVQLVQSGGGVKKIGAAVRISCEVTGYKFMDQLINWVRQAPGQGLEWMGWMNPTYGQVNYSWRFEGRVTMTRQLSQDPDDPDWGTAFMELRGLRVDDTAVYYCARGPSGENYPFHYWGQGVRVVVSSPSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 506 - DNA, 1371 bp, synthetic construct
  misc_feature 1..1371: note = antibody heavy chain
  source 1..1371: mol_type = other DNA
cacgtccaattggtgcagtctggaggtggggtgaagaagattggggccgctgtgaggatctcctgcgaggtgactggatataaattcatggaccaactcataaactgggtgcggcaggcccccggtcagggccttgagtggatgggatggatgaatccaacatatggacaagtaaattattcatggagatttgaaggaagggtcaccatgaccaggcaattatctcaagacccagacgacccggactggggcacggccttcatggagttgagaggactgagagtggacgacacggccgtctattattgcgcgaggggaccctctggggaaaattatccttttcactattggggccagggtgtccgagtggtcgtctcgtcaccctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 507 - protein, 457 aa, synthetic construct
  REGION 1..457: note = antibody heavy chain
HVQLVQSGGGVKKIGAAVRISCEVSGYNFMDQFINWVRQAPGQGLEWMGWMNPIYGQVNYSWRFQGRVTMTRQLSQDPDDPDWGTAFMELRGLRVDDTAVYYCARGPSGENYPFHYWGQGVRVVVSSPSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 508 - DNA, 1371 bp, synthetic construct
  misc_feature 1..1371: note = antibody heavy chain
  source 1..1371: mol_type = other DNA
cacgtccaattggtgcagtctggaggtggggtgaagaagattggggccgctgtgaggatctcctgcgaggtgtctggatacaacttcatggaccaattcataaattgggtgcgacaggcccccggtcagggccttgagtggatgggatggatgaacccaatatatggacaagtaaattattcatggagatttcaaggaagggtcaccatgaccaggcaattatctcaagacccagacgacccggactggggcacggccttcatggagttgagaggactgagagtggacgacacggccgtctattattgcgcgaggggaccctctggggaaaattatccttttcactattggggccagggtgtccgagtggtcgtctcgtcaccctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 509 - protein, 457 aa, synthetic construct
  REGION 1..457: note = antibody heavy chain
HVQLVQSGGGVKKIGAAVRISCEVSGYNFMDQFINWVRQAPGQGLEWMGWMNPIYGQVNYSWRFQGRVTMTRQLSQDPDDPDWGTAFMELRGLRVDDTAVYYCARGPSGENYPFHYWGQGVRVVVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 510 - DNA, 1371 bp, synthetic construct
  misc_feature 1..1371: note = antibody heavy chain
  source 1..1371: mol_type = other DNA
cacgtccaattggtgcagtctggaggtggggtgaagaagattggggccgctgtgaggatctcctgcgaggtgtctggatacaacttcatggaccaattcataaattgggtgcgacaggcccccggtcagggccttgagtggatgggatggatgaacccaatatatggacaagtaaattattcatggagatttcaaggaagggtcaccatgaccaggcaattatctcaagacccagacgacccggactggggcacggccttcatggagttgagaggactgagagtggacgacacggccgtctattattgcgcgaggggaccctctggggaaaattatccttttcactattggggccagggtgtccgagtggtcgtctcgtcagcgtccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 511 - protein, 203 aa, synthetic construct
  REGION 1..203: note = antibody light chain
LTQPASMSASPGQSVTISCSGTRHIISAWFQQYPGKPPKLIIFDDDKRPSGVPSRFSASRPGDTASLTISNVQPEDEATYICNTYEFFGGGTKLTVLGQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 512 - DNA, 609 bp, synthetic construct
  misc_feature 1..609: note = antibody light chain
  source 1..609: mol_type = other DNA
ctgactcagcctgcctccatgtctgcgtcccctggacagtcggtaaccatctcgtgctctggaaccagacacataatctctgcttggttccaacaatatccaggcaaaccacccaaactcataatttttgacgacgataagcgtccctctggagttcctagtcgcttctctgcctccaggcctggcgacacggcctccctgacaatctctaatgttcaacctgaggacgaggcgacatacatttgcaatacatatgaattctttggcggagggaccaaattgaccgtcctaggtcagcccaaggctgccccctcggtcactctgttcccaccctcctctgaggagcttcaagccaacaaggccacactggtgtgtctcgtaagtgacttctacccgggagccgtgacagtggcctggaaggcagatggcagccccgtcaaggtgggagtggagaccaccaaaccctccaaacaaagcaacaacaagtatgcggccagcagctacctgagcctgacgcccgagcagtggaagtcccacagaagctacagctgccgggtcacgcatgaagggagcaccgtggagaagacagtggcccctgcagaatgctct

SEQ ID NO: 513 - protein, 457 aa, synthetic construct
  REGION 1..457: note = antibody heavy chain
HVQLVQSGGGVKKIGAAVRISCEVSGYNFMDQFINWVRQAPGQGLEWMGWMNPIWGQVNYSWRFQGRVTMTRQLSQDPDDPDWGTAFMELRGLRVDDTAVYYCARGPSGENYPFHYWGQGVRVVVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 514 - DNA, 1371 bp, synthetic construct
  misc_feature 1..1371: note = antibody heavy chain
  source 1..1371: mol_type = other DNA
cacgtccaattggtgcagtctggaggtggggtgaagaagattggggccgctgtgaggatctcctgcgaggtgtctggatacaacttcatggaccaattcataaattgggtgcgacaggcccccggtcagggccttgagtggatgggatggatgaacccaatatggggacaagtaaattattcatggagatttcaaggaagggtcaccatgaccaggcaattatctcaagacccagacgacccggactggggcacggccttcatggagttgagaggactgagagtggacgacacggccgtctattattgcgcgaggggaccctctggggaaaattatccttttcactattggggccagggtgtccgagtggtcgtctcgtcagcgtccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 515 - protein, 203 aa, synthetic construct; identical to SEQ ID NO: 511
  REGION 1..203: note = antibody light chain

SEQ ID NO: 516 - DNA, 609 bp, synthetic construct; identical to SEQ ID NO: 512
  misc_feature 1..609: note = Antibody light chain
  source 1..609: mol_type = other DNA

SEQ ID NO: 517 - protein, 457 aa, synthetic construct
  REGION 1..457: note = Antibody heavy chain
HVQLVQSGGGVKKIGAAVRISCEVSGYNFMDQFINWVRQAPGQGLEWMGWMNPIFGQVNYSWRFQGRVTMTRQLSQDPDDPDWGTAFMELRGLRVDDTAVYYCARGPSGENYPFHYWGQGVRVVVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 518 - DNA, 1371 bp, synthetic construct
  misc_feature 1..1371: note = antibody heavy chain
  source 1..1371: mol_type = other DNA
cacgtccaattggtgcagtctggaggtggggtgaagaagattggggccgctgtgaggatctcctgcgaggtgtctggatacaacttcatggaccaattcataaattgggtgcgacaggcccccggtcagggccttgagtggatgggatggatgaacccaatatttggacaagtaaattattcatggagatttcaaggaagggtcaccatgaccaggcaattatctcaagacccagacgacccggactggggcacggccttcatggagttgagaggactgagagtggacgacacggccgtctattattgcgcgaggggaccctctggggaaaattatccttttcactattggggccagggtgtccgagtggtcgtctcgtcagcgtccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 519 - protein, 203 aa, synthetic construct; identical to SEQ ID NO: 511
  REGION 1..203: note = antibody light chain

SEQ ID NO: 520 - DNA, 609 bp, synthetic construct; identical to SEQ ID NO: 512
  misc_feature 1..609: note = antibody light chain
  source 1..609: mol_type = other DNA

SEQ ID NO: 521 - protein, 457 aa, synthetic construct
  REGION 1..457: note = antibody heavy chain
HVQLVQSGGGVKKIGAAVRISCEVSGYNFMDQFINWVRQAPGQGLEWMGWMNPIYGQVNYSWRFQGRVTMTRLFSQDLYYPDRGTAFMELRGLRVDDTAVYYCARGPSGENYPFHYWGQGVRVVVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 522 - DNA, 1371 bp, synthetic construct
  misc_feature 1..1371: note = antibody heavy chain
  source 1..1371: mol_type = other DNA
cacgtccaattggtgcagtctggaggtggggtgaagaagattggggccgctgtgaggatctcctgcgaggtgtctggatacaacttcatggaccaattcataaattgggtgcgacaggcccccggtcagggccttgagtggatgggatggatgaacccaatatatggacaagtaaattattcatggagatttcaaggaagggtcaccatgaccaggctattctctcaagacctatactacccggaccggggcacggccttcatggagttgagaggactgagagtggacgacacggccgtctattattgcgcgaggggaccctctggggaaaattatccttttcactattggggccagggtgtccgagtggtcgtctcgtcagcgtccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 523 - protein, 203 aa, synthetic construct; identical to SEQ ID NO: 511
  REGION 1..203: note = antibody light chain

SEQ ID NO: 524 - DNA, 609 bp, synthetic construct; identical to SEQ ID NO: 512
  misc_feature 1..609: note = antibody light chain
  source 1..609: mol_type = other DNA

SEQ ID NO: 525 - protein, 457 aa, synthetic construct
  REGION 1..457: note = antibody heavy chain
HVQLVQSGGGVKKIGAAVRISCEVSGYDFMDQFINWVRQAPGQGLEWMGWMNPIYGQVNYSWRFQGRVTMTRQLSQDPDDPDWGTAFMELRGLRVDDTAVYYCARGPSGENYPFHYWGQGVRVVVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 526 - DNA, 1371 bp, synthetic construct
  misc_feature 1..1371: note = antibody heavy chain
  source 1..1371: mol_type = other DNA
cacgtccaattggtgcagtctggaggtggggtgaagaagattggggccgctgtgaggatctcctgcgaggtgtctggatacgacttcatggaccaattcataaattgggtgcgacaggcccccggtcagggccttgagtggatgggatggatgaacccaatatatggacaagtaaattattcatggagatttcaaggaagggtcaccatgaccaggcaattatctcaagacccagacgacccggactggggcacggccttcatggagttgagaggactgagagtggacgacacggccgtctattattgcgcgaggggaccctctggggaaaattatccttttcactattggggccagggtgtccgagtggtcgtctcgtcagcgtccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 527 - protein, 203 aa, synthetic construct; identical to SEQ ID NO: 511
  REGION 1..203: note = antibody light chain

SEQ ID NO: 528 - DNA, 609 bp, synthetic construct; identical to SEQ ID NO: 512
  misc_feature 1..609: note = antibody light chain
  source 1..609: mol_type = other DNA

SEQ ID NO: 529 - protein, 457 aa, synthetic construct
  REGION 1..457: note = antibody heavy chain
HVQLVQSGGGVKKIGAAVRISCEVSGYDFIDQFINWVRQAPGQGLEWMGWMNPIYGQVNYSWRFQGRVTMTRQLSQDPDDPDWGTAFMELRGLRVDDTAVYYCARGPSGENYPFHYWGQGVRVVVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 530 - DNA, 1371 bp, synthetic construct
  misc_feature 1..1371: note = antibody heavy chain
  source 1..1371: mol_type = other DNA
cacgtccaattggtgcagtctggaggtggggtgaagaagattggggccgctgtgaggatctcctgcgaggtgtctggatacgacttcattgaccaattcataaattgggtgcgacaggcccccggtcagggccttgagtggatgggatggatgaacccaatatatggacaagtaaattattcatggagatttcaaggaagggtcaccatgaccaggcaattatctcaagacccagacgacccggactggggcacggccttcatggagttgagaggactgagagtggacgacacggccgtctattattgcgcgaggggaccctctggggaaaattatccttttcactattggggccagggtgtccgagtggtcgtctcgtcagcgtccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 531 - protein, 203 aa, synthetic construct; identical to SEQ ID NO: 511
  REGION 1..203: note = antibody light chain

SEQ ID NO: 532 - DNA, 609 bp, synthetic construct; identical to SEQ ID NO: 512
  misc_feature 1..609: note = antibody light chain
  source 1..609: mol_type = other DNA

SEQ ID NO: 533 - protein, 457 aa, synthetic construct
  REGION 1..457: note = antibody heavy chain
HVQLVQSGGGVKKIGAAVRISCEVSGYNFMDQFINWVRQAPGQGLEWMGWMNPIYGQVNYSWRFQGRVTMTRQLSQDPDDPDWGTAFMELRGLRVDDTAVYYCARGPSGENYPFHYWGQGVRVVVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 534 - DNA, 1371 bp, synthetic construct
  misc_feature 1..1371: note = antibody heavy chain
  source 1..1371: mol_type = other DNA
cacgtccaattggtgcagtctggaggtggggtgaagaagattggggccgctgtgaggatctcctgcgaggtgtctggatacaacttcatggaccaattcataaattgggtgcgacaggcccccggtcagggccttgagtggatgggatggatgaacccaatatatggacaagtaaattattcatggagatttcaaggaagggtcaccatgaccaggcaattatctcaagacccagacgacccggactggggcacggccttcatggagttgagaggactgagagtggacgacacggccgtctattattgcgcgaggggaccctctggggaaaattatccttttcactattggggccagggtgtccgagtggtcgtctcgtcagcgtccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgctgcatgaggctctgcacagccactacacgcagaagagcctctccctgtctccgggtaaa

SEQ ID NO: 535 - protein, 203 aa, synthetic construct; identical to SEQ ID NO: 511
  REGION 1..203: note = antibody light chain

SEQ ID NO: 536 - DNA, 609 bp, synthetic construct; identical to SEQ ID NO: 512
  misc_feature 1..609: note = antibody light chain
  source 1..609: mol_type = other DNA

SEQ ID NO: 537 - protein, 457 aa, synthetic construct
  REGION 1..457: note = antibody heavy chain
HVQLVQSGGGVKKIGAAVRISCEVSGYNFMDQFINWVRQAPGQGLEWMGWMNPIYGQVNYSWRFQGRVTMTRQLSQDPDDPDWGTAFMELRGLRVDDTAVYYCARGPSGENYPFHYWGQGVRVVVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 538 - DNA, 1371 bp, synthetic construct
  misc_feature 1..1371: note = antibody heavy chain
  source 1..1371: mol_type = other DNA
cacgtccaattggtgcagtctggaggtggggtgaagaagattggggccgctgtgaggatctcctgcgaggtgtctggatacaacttcatggaccaattcataaattgggtgcgacaggcccccggtcagggccttgagtggatgggatggatgaacccaatatatggacaagtaaattattcatggagatttcaaggaagggtcaccatgaccaggcaattatctcaagacccagacgacccggactggggcacggccttcatggagttgagaggactgagagtggacgacacggccgtctattattgcgcgaggggaccctctggggaaaattatccttttcactattggggccagggtgtccgagtggtcgtctcgtcagcgtccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctctatatcacccgggagcctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 539 - protein, 203 aa, synthetic construct; identical to SEQ ID NO: 511
  REGION 1..203: note = antibody light chain

SEQ ID NO: 540 - DNA, 609 bp, synthetic construct; identical to SEQ ID NO: 512
  misc_feature 1..609: note = antibody light chain
  source 1..609: mol_type = other DNA

SEQ ID NO: 541 - protein, 12 aa, synthetic construct
  REGION 1..12: note = antibody heavy chain sequence
QLSQDPDDPDWG

SEQ ID NO: 542 - protein, 12 aa, synthetic construct
  REGION 1..12: note = antibody heavy chain sequence
LFSQDLYYPDRG

SEQ ID NO: 543 - protein, 8 aa, synthetic construct
  REGION 1..8: note = CDR
MNPIWGQV

SEQ ID NO: 544 - protein, 8 aa, synthetic construct
  REGION 1..8: note = CDR
MNPIFGQV

SEQ ID NO: 545 - protein, 8 aa, synthetic construct
  REGION 1..8: note = CDR
GYDFMDQF

SEQ ID NO: 546 - protein, 8 aa, synthetic construct
  REGION 1..8: note = CDR
GYDFIDQF